(19) United States
(12) Patent Application Publication
(10) Pub. No.: US 2006/0173183 A1
(43) Pub. Date: Aug. 3, 2006

(54) Multicyclic bis-amide MMP inhibitors

(75) Inventors: Timothy Powers, Boston, MA (US); Christoph Steeneck, Dossenheim (DE); Ralf Biesinger, Ludwigshafen (DE); Harald Bluhm, Dossenheim (DE); Hongbo Deng, Southborough, MA (US); Rory Dodd, Watertown, MA (US); Brian M. Gallagher JR., Merrimac, MA (US); Christian Gege, Mauer (DE); Matthias Hochgurtel, Schriesheim (DE); Andrew Kiely, Newton, MA (US); Frank Richter, Handschuhsheim (DE); Matthias Schneider, Dossenheim (DE); Irving Sucholeiki, Winchester, MA (US); Joshua Van Veldhuizen, Brookline, MA (US); Xinyuan Wu, Newton, MA (US); Arthur G. Taveras, Southborough, MA (US)

Correspondence Address: HOFFMANN & BARON, LLP, 6900 JERICHO TURNPIKE, SYOSSET, NY, 11791, US

(73) Assignee: Alantos Pharmaceuticals, Inc.,

(21) Appl. No.: 11/324,037

(22) Filed: Dec. 30, 2005

Related U.S. Application Data
(60) Provisional application No. 60/640,795, filed on Dec. 31, 2004.
(60) Provisional application No. 60/706,267, filed on Aug. 8, 2005.

(51) Int. Cl. C07D 417/02; C07D 403/02; C07D 413/02

(52) U.S. Cl. 544/331; 544/333

(57) ABSTRACT

The present invention relates generally to bis-amide group containing pharmaceutical agents, and in particular, to multicyclic bis-amide MMP-13 inhibitor compounds. More particularly, the present invention provides a new class of MMP-13 inhibiting compounds, containing a pyrimidinyl bis-amide group in combination with a heterocyclic moiety, that exhibit an increased potency and solubility in relation to currently known bis-amide group containing MMP-13 inhibitors.

### CROSS-REFERENCE TO RELATED APPLICATIONS

This application claims the benefit of U.S. Provisional Application No. 60/640,795, filed Dec. 31, 2004, and U.S. Provisional Application No. 60/706,267, filed Aug. 8, 2005, the contents of which are incorporated herein by reference.

### FIELD OF THE INVENTION

The present invention relates generally to bis-amide containing MMP inhibiting compounds, and more particularly to multicyclic bis-amide MMP-13 inhibiting compounds.

### BACKGROUND OF THE INVENTION

Matrix metalloproteinases (MMPs) are a family of structurally related zinc-containing enzymes that have been reported to mediate the breakdown of connective tissue in normal physiological processes such as embryonic development, reproduction, and tissue remodeling. Over-expression of MMPs or an imbalance between MMPs has been suggested as factors in inflammatory, malignant and degenerative disease processes characterized by the breakdown of extracellular matrix or connective tissues. MMPs are, therefore, targets for therapeutic inhibitors in several inflammatory, malignant and degenerative diseases such as rheumatoid arthritis, osteoarthritis, osteoporosis, periodontitis, multiple sclerosis, gingivitis, corneal epidermal and gastric ulceration, atherosclerosis, neointimal proliferation (which leads to restenosis and ischemic heart failure) and tumor metastasis.

The mammalian MMP family has been reported to include at least 20 enzymes, (Chem. Rev. 1999, 99, 2735-2776). Collagenase-3 (MMP-13) is among three collagenases that have been identified. Based on identification of domain structures for individual members of the MMP family, it has been determined that the catalytic domain of the MMPs contains two zinc atoms; one of these zinc atoms performs a catalytic function and is coordinated with three histidines contained within the conserved amino acid sequence of the catalytic domain. MMP-13 is over-expressed in rheumatoid arthritis, osteoarthritis, abdominal aortic aneurysm, breast carcinoma, squamous cell carcinomas of the head and neck, and vulvar squamous cell carcinoma. The principal substrates of MMP-13 are fibrillar collagens (types I, II, III) and gelatins, proteoglycans, cytokines and other components of ECM (extracellular matrix).

The activation of the MMPs involves the removal of a propeptide portion, which features an unpaired cysteine residue catalytic zinc (II) ion. X-ray crystal structures of the complex between MMP-3 catalytic domain and TIMP-1 and MMP-14 catalytic domain and TIMP-2 also reveal ligation of the catalytic zinc (II) ion by the thiol of a cysteine residue. The difficulty in developing effective MMP inhibiting compounds is compounded by several factors, including choice of selective versus broad-spectrum MMP inhibiting activity and rendering such compounds bioavailable via an oral route of administration.

A series of MMP-13 inhibiting compounds containing a bis-amide functional group in combination with a pyridine ring is disclosed in WO 02/064568, while WO 03/049738 discloses that certain bis-amide compounds containing a pyridine and pyrimidine ring and terminally substituted with phenyl rings that exhibit selective inhibition of MMP-13 enzymes. However, many of those compounds exhibit relatively low potencies, and therefore require higher doses for effective MMP-13 inhibition to enable their utilization for the treatment of symptoms and diseases mediated by MMP-13.

### SUMMARY OF THE INVENTION

The present invention relates to a new class of multicyclic bis-amide containing pharmaceutical agents. In particular, the present invention provides a new class of MMP-13 inhibiting compounds containing a pyrimidinyl bis-amide group in combination with a multicyclic moiety that exhibit potent MMP-13 inhibiting activity and are highly selective toward MMP-13 compared to currently known MMP inhibitors.

The present invention provides a new class of multicyclic bis-amide MMP-13 inhibiting compounds that are represented by the general Formula (I):

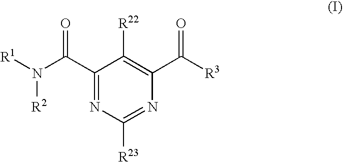

wherein:

R¹ is selected from alkyl, cycloalkyl-alkyl, arylalkyl, heteroarylalkyl and CHR²⁵R²¹, wherein alkyl, cycloalkyl-alkyl, arylalkyl and heteroarylalkyl are optionally substituted one or more times;

R² is hydrogen;

R³ is NR²⁰R²¹;

R¹⁰ and R¹¹ are independently selected from hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or R¹⁰ and R¹¹ when taken together with the nitrogen to which they are attached complete a 3- to 8-membered ring containing carbon atoms and optionally containing a heteroatom selected from O, S, or NR⁵⁰ and which is optionally substituted one or more times;

R²⁰ is selected from hydrogen and alkyl, wherein alkyl is optionally substituted one or more times;

R²¹ is a bicyclic or tricyclic fused ring system, wherein at least one ring is partially saturated, and wherein said bicyclic or tricyclic fused ring system is optionally substituted one or more times;

R²² and R²³ are independently selected from hydrogen, halo, alkyl, cycloalkyl, hydroxy, alkoxy, aryl, heteroaryl, arylalkyl, heteroarylalkyl, alkenyl, alkynyl, NO₂, NR¹⁰R¹¹, NR¹⁰NR¹⁰R¹¹, NR¹⁰N═CR¹⁰R¹¹, NR¹⁰SO₂R¹¹, CN, C(O)OR¹⁰, and fluoroalkyl, wherein alkyl, cycloalkyl, alkoxy, alkenyl, alkynyl and fluoroalkyl are optionally substituted one or more times;

R²⁵ is selected from hydrogen, alkyl, cycloalkyl, C(O)NR¹⁰R¹¹ and haloalkyl, wherein alkyl, cycloalkyl, and haloalkyl are optionally substituted one or more times;

R⁵⁰ is selected from hydrogen, alkyl, aryl, heteroaryl, C(O)R⁸⁰, C(O)NR⁸⁰R⁸¹, SO₂R⁸⁰ and SO₂NR⁸⁰R⁸¹, wherein alkyl, aryl and heteroaryl are optionally substituted one or more times;

R⁸⁰ and R⁸¹ are independently selected from hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or R⁸⁰ and R⁸¹ when taken together with the nitrogen to which they are attached complete a 3- to 8-membered ring containing carbon atoms and optionally a heteroatom selected from O, S(O)ₓ, —NH, and —N(alkyl) and which is optionally substituted one or more times;

x is selected from 0-2; and

N-oxides, pharmaceutically acceptable salts, and stereoisomers therof.

The multicyclic bis-amide MMP-13 inhibiting compounds of the present invention may be used in the treatment of MMP-13 mediated osteoarthritis and may be used for other MMP-13 mediated symptoms, inflammatory, malignant and degenerative diseases characterized by excessive extracellular matrix degradation and/or remodeling, such as cancer, and chronic inflammatory diseases such as arthritis, rheumatoid arthritis, osteoarthritis atherosclerosis, abdominal aortic aneurysm, inflammation, multiple sclerosis, and chronic obstructive pulmonary disease, and pain, such as inflammatory pain, bone pain and joint pain.

The present invention also provides multicyclic bis-amide MMP-13 inhibiting compounds that are useful as active ingredients in pharmaceutical compositions for treatment or prevention of MMP-13 mediated diseases. The present invention also contemplates use of such compounds in pharmaceutical compositions for oral or parenteral administration, comprising one or more of the multicyclic bis-amide MMP-13 inhibiting compounds disclosed herein.

The present invention further provides methods of inhibiting MMP-13, by administering formulations, including, but not limited to, oral, intravenous, parenteral or intraarticular formulations, comprising the multicyclic bis-amide MMP-13 inhibiting compounds by standard methods known in medical practice, for the treatment of diseases or symptoms arising from or associated with MMP-13, including prophylactic and therapeutic treatment.

The multicyclic bis-amide MMP-13 inhibiting compounds of the present invention may be used in combination with a disease modifying antirheumatic drug, a nonsteroidal anti-inflammatory drug, a COX-2 selective inhibitor, a COX-1 inhibitor, an immunosuppressive, a steroid, a biological response modifier or other anti-inflammatory agents or therapeutics useful for the treatment of chemokine mediated diseases.

### DETAILED DESCRIPTION OF THE INVENTION

The terms “alkyl” or “alk”, as used herein alone or as part of another group, denote optionally substituted, straight and branched chain saturated hydrocarbon groups, preferably having 1 to 10 carbons in the normal chain, most preferably lower alkyl groups. Exemplary unsubstituted such groups include methyl, ethyl, propyl, isopropyl, n-butyl, t-butyl, isobutyl, pentyl, hexyl, isohexyl, heptyl, 4,4-dimethylpentyl, octyl, 2,2,4-trimethylpentyl, nonyl, decyl, undecyl, dodecyl and the like. Exemplary substituents may include, but are not limited to, one or more of the following groups: halo, alkoxy, alkylthio, alkenyl, alkynyl, aryl (e.g., to form a benzyl group), cycloalkyl, cycloalkenyl, hydroxy or protected hydroxy, carboxyl (—COOH), alkyloxycarbonyl, alkylcarbonyloxy, alkylcarbonyl, carbamoyl (NH₂—CO—), substituted carbamoyl ((R¹⁰)(R¹¹)N—CO— wherein R¹⁰ or R¹¹ are as defined below, except that at least one of R¹⁰ or R¹¹ is not hydrogen), amino, heterocyclo, mono- or dialkylamino, or thiol (—SH).

The terms “lower alk” or “lower alkyl” as used herein, denote such optionally substituted groups as described above for alkyl having 1 to 4 carbon atoms in the normal chain.

The term “alkoxy” denotes an alkyl group as described above bonded through an oxygen linkage (—O—).

The term “alkenyl”, as used herein alone or as part of another group, denotes optionally substituted, straight and branched chain hydrocarbon groups containing at least one carbon to carbon double bond in the chain, and preferably having 2 to 10 carbons in the normal chain. Exemplary unsubstituted such groups include ethenyl, propenyl, isobutenyl, butenyl, pentenyl, hexenyl, heptenyl, octenyl, nonenyl, decenyl, and the like. Exemplary substituents may include, but are not limited to, one or more of the following groups: halo, alkoxy, alkylthio, alkyl, alkynyl, aryl, cycloalkyl, cycloalkenyl, hydroxy or protected hydroxy, carboxyl (—COOH), alkyloxycarbonyl, alkylcarbonyloxy, alkylcarbonyl, carbamoyl (NH₂—CO—), substituted carbamoyl ((R¹⁰)(R¹¹)N—CO— wherein R¹⁰ or R¹¹ are as defined below, except that at least one of R¹⁰ or R¹¹ is not hydrogen), amino, heterocyclo, mono- or dialkylamino, or thiol (—SH).

The term “alkynyl”, as used herein alone or as part of another group, denotes optionally substituted, straight and branched chain hydrocarbon groups containing at least one carbon to carbon triple bond in the chain, and preferably having 2 to 10 carbons in the normal chain. Exemplary unsubstituted such groups include, but are not limited to, ethynyl, propynyl, butynyl, pentynyl, hexynyl, heptynyl, octynyl, nonynyl, decynyl, and the like. Exemplary substituents may include, but are not limited to, one or more of the following groups: halo, alkoxy, alkylthio, alkyl, alkenyl, aryl, cycloalkyl, cycloalkenyl, hydroxy or protected hydroxy, carboxyl (—COOH), alkyloxycarbonyl, alkylcarbonyloxy, alkylcarbonyl, carbamoyl (NH₂—CO—), substituted carbamoyl ((R¹⁰)(R¹¹)N—CO— wherein R¹⁰ or R¹¹ are as defined below, except that at least one of R¹⁰ or R¹¹ is not hydrogen), amino, heterocyclo, mono- or dialkylamino, or thiol (—SH).

The term “cycloalkyl”, as used herein alone or as part of another group, denotes optionally substituted, saturated cyclic hydrocarbon ring systems, including bridged ring systems, desirably containing 1 to 3 rings and 3 to 9 carbons per ring. Exemplary unsubstituted such groups include, but are not limited to, cyclopropyl, cyclobutyl, cyclopentyl, cyclohexyl, cycloheptyl, cyclooctyl, cyclodecyl, cyclododecyl, and adamantyl. Exemplary substituents include, but are not limited to, one or more alkyl groups as described above, or one or more groups described above as alkyl substituents.

The terms “ar” or “aryl”, as used herein alone or as part of another group, denote optionally substituted, homocyclic aromatic groups, preferably containing 1 or 2 rings and 6 to 12 ring carbons. Exemplary unsubstituted such groups include, but are not limited to, phenyl, biphenyl, and naphthyl. Exemplary substituents include, but are not limited to, one or more nitro groups, alkyl groups as described above or groups described above as alkyl substituents.

The term “heterocycle” or “heterocyclic system” denotes a heterocyclyl, heterocyclenyl, or heteroaryl group as described herein, which contains carbon atoms and from 1 to 4 heteroatoms independently selected from N, 0 and S and including any bicyclic or tricyclic group in which any of the above-defined heterocyclic rings is fused to one or more heterocycle, aryl or cycloalkyl groups. The nitrogen and sulfur heteroatoms may optionally be oxidized. The heterocyclic ring may be attached to its pendant group at any heteroatom or carbon atom which results in a stable structure. The heterocyclic rings described herein may be substituted on carbon or on a nitrogen atom.

Examples of heterocycles include, but are not limited to, lH-indazole, 2-pyrrolidonyl, 2H,6H-1,5,2-dithiazinyl, 2H-pyrrolyl, 3H-indolyl, 4-piperidonyl, 4aH-carbazole, 4H-quinolizinyl, 6H-1,2,5-thiadiazinyl, acridinyl, azocinyl, benzimidazolyl, benzofuranyl, benzothiofuranyl, benzothiophenyl, benzoxazolinyl, benzoxazolyl, benzthiazolyl, benztriazolyl, benztetrazolyl, benzisoxazolyl, benzisothiazolyl, benzimidazalonyl, carbazolyl, 4aH-carbazolyl, b-carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, lH-indazolyl, indolenyl, indolinyl, indolizinyl, indolyl, isatinoyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinylperimidinyl, oxindolyl, phenanthridinyl, phenanthrolinyl, phenarsazinyl, phenazinyl, phenothiazinyl, phenoxathiinyl, phenoxazinyl, phthalazinyl, piperazinyl, piperidinyl, pteridinyl, piperidonyl, 4-piperidonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, 4H-quinolizinyl, quinoxalinyl, quinuclidinyl, carbolinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, xanthenyl.

“Heterocyclenyl” denotes a non-aromatic monocyclic or multicyclic hydrocarbon ring system of about 3 to about 10 atoms, desirably about 4 to about 8 atoms, in which one or more of the carbon atoms in the ring system is/are hetero element(s) other than carbon, for example nitrogen, oxygen or sulfur atoms, and which contains at least one carbon-carbon double bond or carbon-nitrogen double bond. Ring sizes of rings of the ring system may include 5 to 6 ring atoms. The designation of the aza, oxa or thia as a prefix before heterocyclenyl define that at least a nitrogen, oxygen or sulfur atom is present respectively as a ring atom. The heterocyclenyl may be optionally substituted by one or more substituents as defined herein. The nitrogen or sulphur atom of the heterocyclenyl may also be optionally oxidized to the corresponding N-oxide, S-oxide or S,S-dioxide. “Heterocyclenyl” as used herein includes by way of example and not limitation those described in Paquette, Leo A.; “Principles of Modem Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and “J. Am. Chem. Soc. ”, 82:5566 (1960), the contents all of which are incorporated by reference herein. Exemplary monocyclic azaheterocyclenyl groups include, but are not limited to, 1,2,3,4-tetrahydrohydropyridine, 1,2-dihydropyridyl, 1,4-dihydropyridyl, 1,2,3,6-tetrahydropyridine, 1,4,5,6-tetrahydropyrimidine, 2-pyrrolinyl, 3-pyrrolinyl, 2-imidazolinyl, 2-pyrazolinyl, and the like. Exemplary oxaheterocyclenyl groups include, but are not limited to, 3,4-dihydro-2H-pyran, dihydrofuranyl, and fluorodihydrofuranyl. An exemplary multicyclic oxaheterocyclenyl group is 7-oxabicyclo[2.2.1]heptenyl.

“Heterocyclyl,” or “heterocycloalkyl,” denotes a non-aromatic saturated monocyclic or multicyclic ring system of about 3 to about 10 carbon atoms, desirably 4 to 8 carbon atoms, in which one or more of the carbon atoms in the ring system is/are hetero element(s) other than carbon, for example nitrogen, oxygen or sulfur. Ring sizes of rings of the ring system may include 5 to 6 ring atoms. The designation of the aza, oxa or thia as a prefix before heterocyclyl define that at least a nitrogen, oxygen or sulfur atom is present respectively as a ring atom. The heterocyclyl may be optionally substituted by one or more substituents which may be the same or different, and are as defined herein. The nitrogen or sulphur atom of the heterocyclyl may also be optionally oxidized to the corresponding N-oxide, S-oxide or S,S-dioxide.

“Heterocyclyl” as used herein includes by way of example and not limitation those described in Paquette, Leo A.; “Principles of Modem Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and “J. Am. Chem. Soc. ”, 82:5566 (1960). Exemplary monocyclic heterocyclyl rings include, but are not limited to, piperidyl, pyrrolidinyl, piperazinyl, morpholinyl, thiomorpholinyl, thiazolidinyl, 1,3-dioxolanyl, 1,4-dioxanyl, tetrahydrofuranyl, tetrahydrothiophenyl, tetrahydrothiopyranyl, and the like.

“Heteroaryl” denotes an aromatic monocyclic or multicyclic ring system of about 5 to about 10 atoms, in which one or more of the atoms in the ring system is/are hetero element(s) other than carbon, for example nitrogen, oxygen or sulfur. Ring sizes of rings of the ring system include 5 to 6 ring atoms. The “heteroaryl” may also be substituted by one or more subsituents which may be the same or different, and are as defined herein. The designation of the aza, oxa or thia as a prefix before heteroaryl define that at least a nitrogen, oxygen or sulfur atom is present respectively as a ring atom. A nitrogen atom of a heteroaryl may be optionally oxidized to the corresponding N-oxide. Heteroaryl as used herein includes by way of example and not limitation those described in Paquette, Leo A. ; “Principles of Modem Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and “J. Am. Chem. Soc.”, 82:5566 (1960). Exemplary heteroaryl and substituted heteroaryl groups include, but are not limited to, pyrazinyl, thienyl, isothiazolyl, oxazolyl, pyrazolyl, furazanyl, pyrrolyl, 1,2,4-thiadiazolyl, pyridazinyl, quinoxalinyl, phthalazinyl, imidazo[1,2-a]pyridine, imidazo[2,1-b]thiazolyl, benzofurazanyl, azaindolyl, benzimidazolyl, benzothienyl, thienopyridyl, thienopyrimidyl, pyrrolopyridyl, imidazopyridyl, benzoazaindole, 1,2,3-triazinyl, 1,2,4-triazinyl, 1,3,5-triazinyl, benzthiazolyl, dioxolyl, furanyl, imidazolyl, indolyl, indolizinyl, isoxazolyl, isoquinolinyl, isothiazolyl, , oxadiazolyl, oxazinyl, oxiranyl, piperazinyl, piperidinyl, pyranyl, pyrazinyl, pyridazinyl, pyrazolyl, pyridyl, pyrimidinyl, pyrrolyl, pyrrolidinyl, quinazolinyl, quinolinyl, tetrazinyl, tetrazolyl, 1,3,4-thiadiazolyl, 1,2,3-thiadiazolyl, 1,2,4-thiadiazolyl, 1,2,5-thiadiazolyl, thiatriazolyl, thiazinyl, thiazolyl, thienyl, 5-thioxo-1,2,4-diazolyl, thiomorpholino, thiophenyl, thiopyranyl, triazolyl and triazolonyl.

The term “amino” denotes the radical —NH₂ wherein one or both of the hydrogen atoms may be replaced by an optionally substituted hydrocarbon group. Exemplary amino groups include, but are not limited to, n-butylamino, tert-butylamino, methylpropylamino and ethyldimethylamino.

The term “cycloalkylalkyl” denotes a cycloalkyl-alkyl group wherein a cycloalkyl as described above is bonded through an alkyl, as defined above. Cycloalkylalkyl groups may contain a lower alkyl moiety. Exemplary cycloalkylalkyl groups include, but are not limited to, cyclopropylmethyl, cyclopentylmethyl, cyclohexylmethyl, cyclopropylethyl, cyclopentylethyl, cyclohexylpropyl, cyclopropylpropyl, cyclopentylpropyl, and cyclohexylpropyl.

The term “arylalkyl” denotes an aryl group as described above bonded through an alkyl, as defined above.

The term “heteroarylalkyl” denotes a heteroaryl group as described above bonded through an alkyl, as defined above.

The term “heterocyclylalkyl,” or “heterocycloalkylalkyl,” denotes a heterocyclyl group as described above bonded through an alkyl, as defined above.

The terms “halogen”, “halo”, or “hal”, as used herein alone or as part of another group, denote chlorine, bromine, fluorine, and iodine.

The term “haloalkyl” denotes a halo group as described above bonded though an alkyl, as defined above. Fluoroalkyl is an exemplary group.

The term “aminoalkyl” denotes an amino group as defined above bonded through an alkyl, as defined above.

The phrase “bicyclic fused ring system wherein at least one ring is partially saturated” denotes an 8- to 1 3-membered fused bicyclic ring group in which at least one of the rings is non-aromatic. The ring group has carbon atoms and optionally 1-4 heteroatoms independently selected from N, O and S. Illustrative examples include, but are not limited to, indanyl, tetrahydronaphthyl, tetrahydroquinolyl and benzocycloheptyl.

The phrase “tricyclic fused ring system wherein at least one ring is partially saturated” denotes a 9- to 1 8-membered fused tricyclic ring group in which at least one of the rings is non-aromatic. The ring group has carbon atoms and optionally 1-7 heteroatoms independently selected from N, O and S. Illustrative examples include, but are not limited to, fluorene, 10,11-dihydro-5H-dibenzo[a,d]cycloheptene and 2,2a,7,7a-tetrahydro-1H-cyclobuta[a]indene.

The term “pharmaceutically acceptable salts” refers to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as, but not limited to, hydrochloric, hydrobromic, sulfuric, sulfamic, phosphoric, nitric and the like; and the salts prepared from organic acids such as, but not limited to, acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Organic solvents include, but are not limited to, nonaqueous media like ethers, ethyl acetate, ethanol, isopropanol, or acetonitrile. Lists of suitable salts are found in Remington's Pharmaceutical Sciences, 18th ed., Mack Publishing Company, Easton, Pa., 1990, p. 1445, the disclosure of which is hereby incorporated by reference.

The phrase “pharmaceutically acceptable” denotes those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

The term “N-oxide” denotes compounds that can be obtained in a known manner by reacting a compound of the present invention including a nitrogen atom (such as in a pyridyl group) with hydrogen peroxide or a peracid, such as 3-chloroperoxy-benzoic acid, in an inert solvent, such as dichloromethane, at a temperature between about -10-80° C., desirably about 0° C.

“Substituted” is intended to indicate that one or more hydrogens on the atom indicated in the expression using “substituted” is replaced with a selection from the indicated group(s), provided that the indicated atom's normal valency is not exceeded, and that the substitution results in a stable compound. When a substituent is keto (i.e., ═O) group, then 2 hydrogens on the atom are replaced.

Unless moieties of a compound of the present invention are defined as being unsubstituted, the moieties of the compound may be substituted. In addition to any substituents provided above, the moieties of the compounds of the present invention may be optionally substituted with one or more groups independently selected from:

C₁-C₄ alkyl;

C₂-C₄ alkenyl;

C₂-C₄ alkynyl;

CF₃;

halo;

OH;

O—(C₁-C₄ alkyl);

OCH₂F;

OCHF₂;

OCF₃;

OC(O)—(C₁-C₄ alkyl);

OC(O)—(C₁-C₄ alkyl);

OC(O)NH—(C₁-C₄ alkyl);

OC(O)N(C₁-C₄ alkyl)₂;

OC(S)NH—(C₁-C₄ alkyl);

OC(S)N(C₁-C₄ alkyl)₂;

SH;

S—(C₁-C₄ alkyl);

S(O)—(C₁-C₄ alkyl);

S(O)₂—(C₁-C₄ alkyl);

SC(O)—(C₁-C₄ alkyl);

SC(O)O—(C₁-C₄ alkyl);

NH₂;

N(H)—(C₁-C₄ alkyl);

N(C₁-C₄ alkyl)₂;

N(H)C(O)—(C₁-C₄ alkyl);

N(CH₃)C(O)—(C₁-C₄ alkyl);

N(H)C(O)—CF₃;

N(CH₃)C(O)—CF₃;

N(H)C(S)—(C₁-C₄ alkyl);

N(CH₃)C(S)—(C₁-C₄ alkyl);

N(H)S(O)₂—(C₁-C₄ alkyl);

N(H)C(O)NH₂;

N(H)C(O)NH—(C₁-C₄ alkyl);

N(CH₃)C(O)NH—(C₁-C₄ alkyl);

N(H)C(O)N(C₁-C₄ alkyl)₂;

N(CH₃)C(O)N(C₁-C₄ alkyl)₂;

N(H)S(O)₂NH₂);

N(H)S(O)₂NH—(C₁-C₄ alkyl);

N(CH₃)S(O)₂NH—(C₁-C₄ alkyl);

N(H)S(O)₂N(C₁-C₄ alkyl)₂;

N(CH₃)S(O)₂N(C₁-C₄ alkyl)₂;

N(H)C(O)O—(C₁-C₄ alkyl);

N(CH₃)C(O)O—(C₁-C₄ alkyl);

N(H)S(O)₂O—(C₁-C₄ alkyl);

N(CH₃)S(O)₂O—(C₁-C₄ alkyl);

N(CH₃)C(S)NH—(C₁-C₄ alkyl);

N(CH₃)C(S)N(C₁-C₄ alkyl)₂;

N(CH₃)C(S)O—(C₁-C₄ alkyl);

N(H)C(S)NH₂;

NO₂;

CO₂H;

CO₂—(C₁-C₄ alkyl);

C(O)N(H)OH;

C(O)N(CH₃)OH:

C(O)N(CH₃)OH;

C(O)N(CH₃)O—(C₁-C₄ alkyl);

C(O)N(H)—(C₁-C₄ alkyl);

C(O)N(C₁-C₄ alkyl)₂;

C(S)N(H)—(C₁-C₄ alkyl);

C(S)N(C₁-C₄ alkyl)₂;

C(NH)N(H)—(C₁-C₄ alkyl);

C(NH)N(C₁-C₄ alkyl)₂;

C(NCH₃)N(H)—(C₁-C₄ alkyl);

C(NCH₃)N(C₁-C₄ alkyl)₂;

C(O)—(C₁-C₄ alkyl);

C(NH)—(C₁-C₄ alkyl);

C(NCH₃)—(C₁-C₄ alkyl);

C(NOH)—(C₁-C₄ alkyl);

C(NOCH₃)—(C₁-C₄ alkyl);

CN;

CHO;

CH₂OH;

CH₂O—(C₁-C₄ alkyl);

CH₂NH₂;

CH₂N(H)—(C₁-C₄ alkyl);

CH₂N(C₁-C₄ alkyl)₂;

aryl;

heteroaryl;

cycloalkyl; and

heterocyclyl.

In some embodiments of the present invention, the multicyclic bis-amide MMP-13 inhibiting compounds are represented by the general Formula (I):

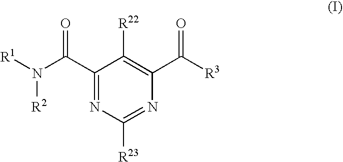

wherein:

R¹ is selected from alkyl, cycloalkyl-alkyl, arylalkyl, heteroarylalkyl and CHR²⁵R²¹, wherein alkyl, cycloalkyl-alkyl, arylalkyl and heteroarylalkyl are optionally substituted one or more times;

R² is hydrogen;

R³ is NR²⁰R²¹;

R¹⁰ and R¹¹ are independently selected from hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or R¹⁰ and R¹¹ when taken together with the nitrogen to which they are attached complete a 3- to 8-membered ring containing carbon atoms and optionally containing a heteroatom selected from O, S, or NR⁵⁰ and which is optionally substituted one or more times;

R²⁰ is selected from hydrogen and alkyl, wherein alkyl is optionally substituted one or more times;

R²¹ is a bicyclic or tricyclic fused ring system, wherein at least one ring is partially saturated, and wherein said bicyclic or tricyclic fused ring system is optionally substituted one or more times;

R²² and R²³ are independently selected from hydrogen, halo, alkyl, cycloalkyl, hydroxy, alkoxy, aryl, heteroaryl, arylalkyl, heteroarylalkyl, alkenyl, alkynyl, NO₂, NR¹⁰R¹¹, NR¹⁰NR¹⁰R¹¹, NR¹⁰N═CR¹⁰R¹¹, NR¹⁰SO₂R¹¹, CN, C(O)OR¹⁰, and fluoroalkyl, wherein alkyl, cycloalkyl, alkoxy, alkenyl, alkynyl and fluoroalkyl are optionally substituted one or more times;

R²⁵ is selected from hydrogen, alkyl, cycloalkyl, C(O)NR¹⁰R¹¹ and haloalkyl, wherein alkyl, cycloalkyl, and haloalkyl are optionally substituted one or more times;

R⁵⁰ is selected from hydrogen, alkyl, aryl, heteroaryl, C(O)R⁸⁰, C(O)NR⁸⁰R⁸¹, SO₂R⁸⁰ and SO₂NR⁸⁰R⁸¹, wherein alkyl, aryl and heteroaryl are optionally substituted one or more times;

R⁸⁰ and R⁸¹ are independently selected from hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or R⁸⁰ and R⁸¹ when taken together with the nitrogen to which they are attached complete a 3- to 8-membered ring containing carbon atoms and optionally a heteroatom selected from O, S(O)ₓ, —NH, and —N(alkyl) and which is optionally substituted one or more times; and

x is selected from 0-2.

Some embodiments of the present invention include N-oxides, pharmaceutically acceptable salts, and stereoisomers of the compounds of Formula (I).

In some embodiments of the present invention, R³ may include a bicyclic ring system. In accordance with such embodiments, R³ may be:

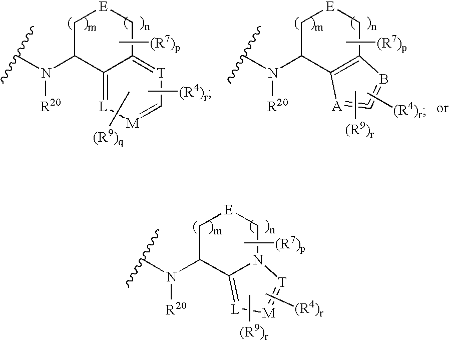

wherein:

R⁴ is selected from R¹⁰, hydrogen, alkyl, aryl, heteroaryl, halo, CF₃, COR¹⁰, OR¹⁰, NR¹⁰R¹¹, NO₂, CN, SO₂OR¹⁰, CO₂R¹⁰, C(O)NR¹⁰R¹¹, SO₂NR¹⁰R¹¹, SO₂R¹⁰, OC(O)R¹⁰, OC(O)NR¹⁰R¹¹, NR¹⁰C(O)R^{11, NR}¹⁰CO₂R¹¹, (C₀-C₆)-alkyl-C(═NR^{a})NHR^{b}, (C₀-C₆)-alkyl-NHC(═NR^{a})NHR^{b}, (C₀-C₆)-alkyl-C(O)OR¹⁰, (C₀-C₆)-alkyl-C(O)NR¹⁰R¹¹, (C₀-C₆)-alkyl-C(O)—NH—CN, O—(C₀-C₆)-alkyl-C(O)NR¹⁰R¹¹, S(O),—(C₀-C₆)-alkyl-C(O)OR¹⁰, S(O),—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰R¹¹, (C₀-C₆)-alkyl-NR¹⁰—C(O)R¹⁰, (C₀-C₆)-alkyl-NR¹⁰-C(O)OR¹⁰, (C₀-C₆)-alkyl-NR¹⁰—C(O)—NR¹⁰R¹¹, (C₀-C₆)-alkyl-NR¹⁰-SO₂NR¹⁰R¹¹, wherein each R⁴ group is optionally substituted by one or more R¹⁴ groups;

R⁵ is selected from hydrogen, alkyl, C(O)NR¹⁰R¹¹, aryl, arylalkyl, SO₂NR¹⁰R¹¹, C(O)OR¹⁰ and CN, wherein alkyl, aryl and arylalkyl are optionally substituted one or more times;

R⁷ is selected from hydrogen, alkyl, cycloalkyl, halo, R⁴ and NR¹⁰R¹¹, wherein alkyl and cycloalkyl are optionally substituted one or more times;

R⁹ is selected from hydrogen, alkyl, CH(CH₃)CO₂H, halo, (C₀-C₆)-alkyl-C(O)OR¹⁰, (C₀-C₆)-alkyl-C(O)NR¹⁰R¹¹, (C₀-C₆)-alkyl-C(O)NH—CN, O—(C₀-C₆)-alkyl-C(O)NR¹⁰R¹¹, S(O)_{y}-alkyl-C(O)OR¹⁰, S(O)_{z}-alkyl-C(O)NR¹⁰R¹¹, (C₀-C₆)-alkyl-C(O)NR¹⁰—(C₀-C₆)-alkyl-NR¹⁰R¹¹, C(O)NR¹⁰—(C₀-C₆)-alkyl-heteroaryl, C(O)NR¹⁰—(C₀-C₆)-alkyl-aryl, CH₂NR¹⁰R¹¹, (CH₂)_{y}NR¹⁰C(O)-alkyl, (CH₂)_{w}NR¹⁰C(O)—(C₀-C₆)-alkyl-aryl, (CH₂)_{w}NR¹⁰C(O)—(C₀-C₆)-alkyl-heteroaryl, (CH₂)_{w}NR¹⁰C(O)O-alkyl, (CH₂)_{w}NR¹⁰C(O)O—(C₀-C₆)-alkyl-aryl, (CH₂)_{w}NR¹⁰C(O)O—(C₀-C₆)-alkyl-heteroaryl, (CH₂)_{w}NR¹⁰C(O)ONR¹⁰R¹¹, (CH₂)_{w}NR¹⁰S(O)₂—(C₀-C₆)-alkyl-aryl, (CH₂)_{w}NR¹⁰S(O)₂—(C₀-C₆)-alkyl-heteroaryl, (CH₂),NR¹⁰S(O)₂-NR¹⁰-alkyl, (CH₂)_{w}NR¹⁰S(O)₂NR¹⁰—(C₀-C₆)-alkyl-aryl, (CH₂)_{w}NR¹⁰S(O)₂NR¹⁰—(C₀-C₆)-alkyl-heteroaryl, (CH₂)_{w}NR¹⁰C(O)NR¹⁰—SO₂—R³⁰, S(O)₂NR¹⁰—(C₀-C₆)-alkyl-aryl, S(O)₂NR¹⁰—(C₀-C₆)-alkyl-heteroaryl, S(O)₂NR¹⁰-alkyl, S(O)₂—(C₀-C₆)-alkyl-aryl, S(O)₂—(C₀-C₆)-alkyl-heteroaryl, O-heteroaryl and heteroaryl, wherein each of said R⁹ groups is optionally substituted one or more times;

R¹⁴ is selected from hydrogen, alkyl, arylalkyl, cycloalkyl-alkyl, heteroarylalkyl, heterocyclylalkyl and halo, wherein alkyl, arylalkyl, cycloalkyl-alkyl, heteroarylalkyl and heterocyclylalkyl are optionally substituted one or more times;

R³⁰ is selected from alkyl and (C₀-C₆)-alkyl-aryl;

R^{a} and R^{b} are independently selected from hydrogen, CN, alkyl, haloalkyl, S(O)ₓNR¹⁰R¹¹, S(O)ₓR¹⁰ and C(O)NR¹⁰R¹¹, wherein alkyl and haloalkyl are optionally substituted one or more times;

E is selected from a bond, CR¹⁰R¹¹, O, NR⁵, S, S═O, S(═O)₂, C(═O), N(R¹⁰)(C═O), (C═O)N(R¹⁰), N(R¹⁰)S(═O)₂, S(═O)₂N(R¹⁰), C═N—OR¹¹, —C(R¹⁰R¹¹)C(R¹⁰R¹¹)—, —CH₂—W— and

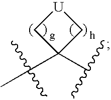

W is selected from O, NR⁵, S, S═O, S(═O)₂, N(R¹⁰)(C═O), N(R¹⁰)S(═O)₂ and S(═O)₂N(R¹⁰);

U is selected from C(R⁵R¹⁰), NR⁵, O, S, S═O and S(═O)₂;

A and B are independently selected from C, N, O and S;

L, M and T are independently selected from C and N;

g and h are independently selected from 0-2;

m and n are independently selected from 0-3, provided that:
(1) when E is present, m and n are not both 3;
(2) when E is —CH₂—W—, m and n are not 3; and
(3) when E is a bond, m and n are not 0;

p is selected from 0-6;

q is selected from 0-4;

r is selected from 0-1;

w is selected from 0-4;

x is selected from 0-2;

y is selected from 1 and 2;

z is selected from 0-2; and

wherein the dotted line represents optionally a double bond.

All remaining variables are as defined above.

In some embodiments of the present invention, R¹⁰ and R¹¹ may be optionally substituted with one or more substituents independently selected from halo, CF₃, COR¹⁰, OR¹⁰, NR¹⁰R¹¹, NO₂, CN, SO₂OR¹⁰, CO₂R¹⁰, CONR¹⁰R¹¹, SO₂NR¹⁰R¹¹, SO₂R¹⁰, OC(O)R¹⁰, OC(O)NR¹⁰R¹¹, NR¹⁰C(O)R¹¹ and NR¹⁰CO₂R¹¹.

In some embodiments, R²⁰ when taken with the nitrogen to which it is bound and L together may form a 3- to 8-membered ring containing carbon atoms and optionally containing a heteroatom selected from O, S, or NR⁵⁰ and which ring is optionally substituted.

More specifically, in such bicyclic embodiments, R³ may be, but is not limited to, the following:

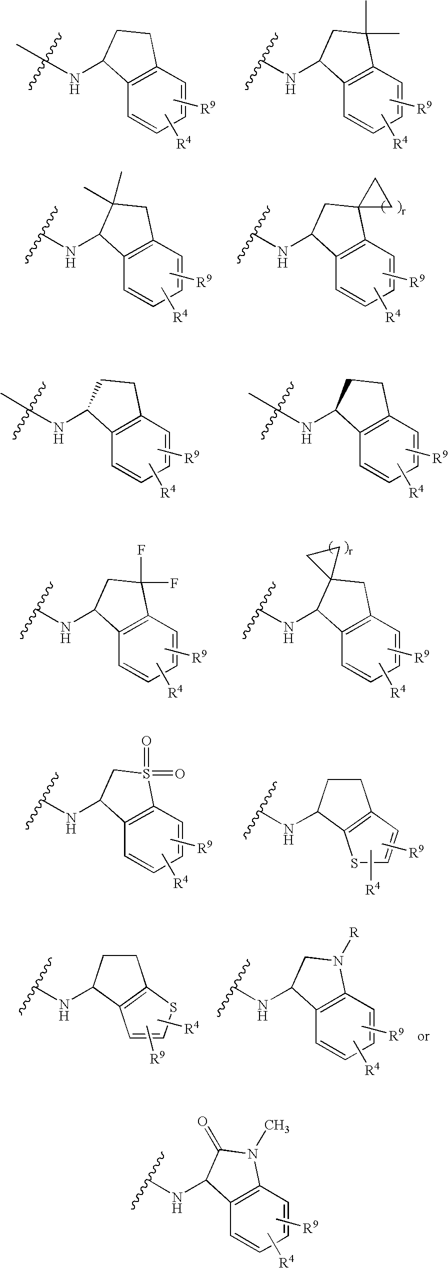

wherein:

R is selected from C(O)NR¹⁰R¹¹, COR¹⁰, SO₂N¹⁰R¹¹, SO₂R¹⁰, CONHCH₃ and CON(CH₃)₂, wherein C(O)NR¹⁰R¹¹, COR¹⁰, SO₂NR¹⁰R¹¹, SO₂R¹⁰, CONHCH₃ and CON(CH₃)₂ are optionally substituted one or more times;

R⁴ is selected from:

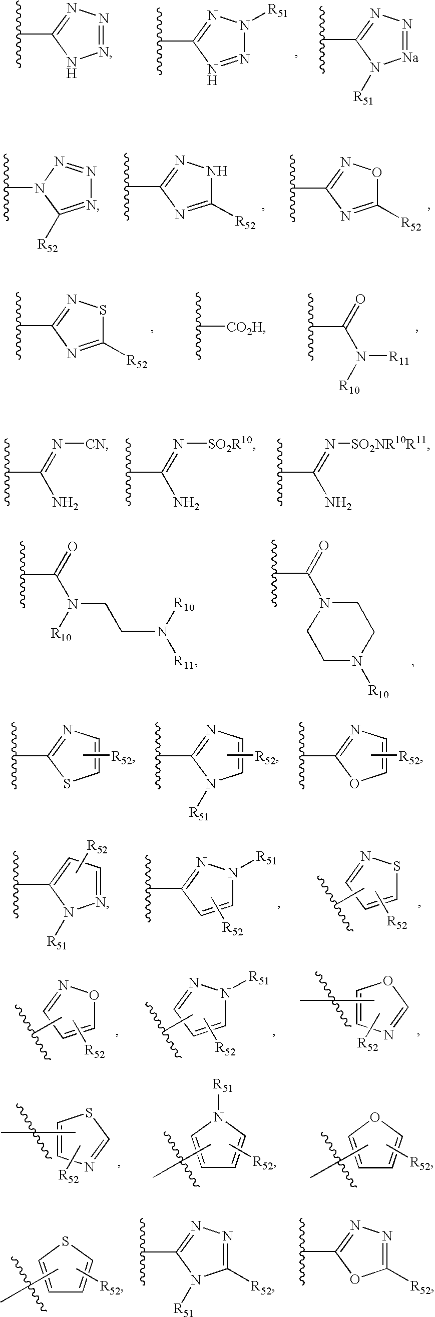

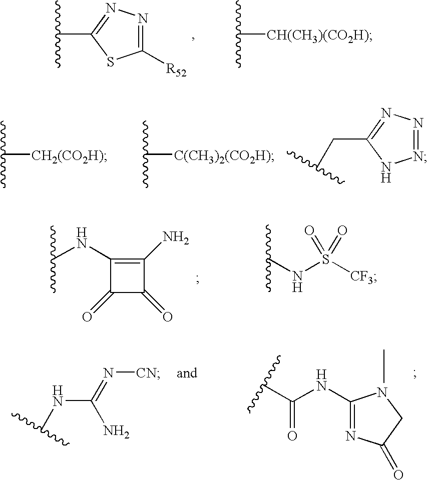

R⁵¹ is selected from hydrogen, alkyl, aryl, heteroaryl, arylalkyl, cycloalkylalkyl, heteroarylalkyl and haloalkyl, wherein alkyl, aryl, heteroaryl, arylalkyl, cycloalkylalkyl, heteroarylalkyl and haloalkyl are optionally substituted one or more times;

R⁵² is selected from hydrogen, halo, hydroxy, alkoxy, fluoroalkoxy, alkyl, aryl, heteroaryl, arylalkyl, cycloalkylalkyl, heteroarylalkyl, haloalkyl, C(O)NR¹⁰R¹¹ and O₂NR¹⁰R¹¹, wherein alkoxy, fluoroalkoxy, alkyl, aryl, heteroaryl, arylalkyl, cycloalkylalkyl, heteroarylalkyl, haloalkyl, C(O)NR¹⁰R¹¹ and O₂NR¹⁰R¹¹ are optionally substituted one or more times; and

r is selected from 0-1.

All remaining variables are as defined above.

In some embodiments of the present invention, when E is present, m and n added together may be 1-4, thereby forming a 5- to 8-membered ring. More desirably, m and n added together may be 1-2, thereby forming a 5- to 6-membered ring.

In other embodiments, when E is a bond, m and n added together may be 2-5, thereby forming a 5- to 8-membered ring. More desirably, m and n added together may be 2-3, thereby forming a 5- to 6-membered ring.

Alternatively, in some embodiments of the present invention, R³ may include a tricyclic ring system. In such embodiments, R³ may be:

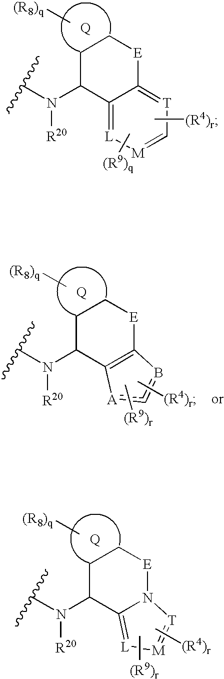

wherein:

R⁴ is selected from R¹⁰, hydrogen, alkyl, aryl, heteroaryl, halo, CF₃, COR¹⁰, OR¹⁰, NR¹⁰R¹¹, NO₂, CN, SO₂OR¹⁰, CO₂R¹⁰, C(O)NR¹⁰R¹¹, SO₂NR¹⁰R¹¹, SO₂R OC(O)NR¹⁰R¹¹, NR¹⁰C(O)R¹¹, NR¹⁰CO₂R¹¹, (C₀-C₆)-alkyl-C(═NR^{a})NHR^{b}, (C₀-C₆)-alkyl-NHC(═NR^{a})NHR^{b}, (C₀-C₆)-alkyl-C(O)OR¹⁰, (C₀-C₆)-alkyl-C(O)NR¹⁰R¹¹, (C₀-C₆)-alkyl-C(O)—NH—CN, O—(C₀-C₆)-alkyl-C(O)NR¹⁰R¹¹, S(O)ₓ—(C₀-C₆)-alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰R¹¹, (C₀-C₆)-alkyl-NR¹⁰-C(O)R¹⁰, (C₀-C₆)-alkyl-NR¹⁰—C(O)OR¹⁰, (C₀-C₆)-alkyl-NR¹⁰-C(O)—NR¹⁰R¹¹, (C₀-C₆)-alkyl-NR¹⁰-SO₂NR¹⁰R¹¹, wherein each R⁴ group is optionally substituted by one or more R¹⁴ groups;

R⁵ is selected from hydrogen, alkyl, C(O)NR¹⁰R¹¹, aryl, arylalkyl, SO₂NR¹⁰R¹¹, C(O)OR¹⁰ and CN, wherein alkyl, aryl and arylalkyl are optionally substituted one or more times;

R⁸ is selected from hydrogen, alkyl, OR¹⁰, NR¹⁰R¹¹, CN, arylalkyl, cycloalkyl-alkyl, heteroarylalkyl, heterocyclylalkyl and halo, wherein alkyl, arylalkyl, cycloalkyl-alkyl, heteroarylalkyl and heterocyclylalkyl are optionally substituted one or more times;

R⁹ is selected from hydrogen, alkyl, CH(CH₃)CO₂H, halo, (C₀-C₆)-alkyl-C(O)OR¹¹, (C₀-C₆)-alkyl-C(O)NR¹⁰R¹¹, (C₀-C₆)-alkyl-C(O)NH—CN, O—(C₀-C₆)-alkyl-C(O)NR¹⁰R¹¹, S(O)_{y}-alkyl-C(O)OR¹⁰, S(O)_{z}-alkyl-C(O)NR¹⁰R¹¹, (C₀-C₆)-alkyl-C(O)NR¹⁰—(C₀-C₆)-alkyl-NR¹⁰R¹¹, C(O)NR¹⁰—(C₀-C₆)-alkyl-heteroaryl, C(O)NR¹⁰—(C₀-C₆)-alkyl-aryl, CH₂NR¹⁰R¹¹, (CH₂)_{y}NR¹⁰C(O)-alkyl, (CH₂)_{w}NR¹⁰C(O)—(C₀-C₆)-alkyl-aryl, (CH₂)_{w}NR¹⁰C(O)—(C₀-C₆)-alkyl-heteroaryl, (CH₂)_{w}NR¹⁰C(O)O-alkyl, (CH₂)_{w}NR¹⁰C(O)O—(C₀-C₆)-alkyl-aryl, (CH₂)_{w}NR¹⁰C(O)O—(C₀-C₆)-alkyl-heteroaryl, (CH₂)_{w}NR¹⁰C(O)ONR¹⁰R¹¹, (CH₂)_{w}NR¹⁰S(O)₂—(C₀-C₆)-alkyl-aryl, (CH₂)_{w}NR¹⁰S(O)₂—(C₀-C₆)-alkyl-heteroaryl, (CH₂)_{w}NR¹⁰S(O)₂—NR¹⁰-alkyl, (CH₂)_{w}NR¹⁰S(O)₂NR¹⁰—(C₀-C₆)-alkyl-aryl, (CH₂)_{w}NR¹⁰S(O)₂NR¹⁰—(C₀-C₆)-alkyl-heteroaryl, (CH₂)_{w}NR¹⁰C(O)NR¹⁰—SO₂—R³⁰, S(O)₂NR¹⁰—(C₀-C₆)-alkyl-aryl, S(O)₂NR¹⁰—(C₀-C₆)-alkyl-heteroaryl, S(O)₂NR¹⁰-alkyl, S(O)₂—(C₀-C₆)-alkyl-aryl, S(O)₂—(C₀-C₆)-alkyl-heteroaryl, O-heteroaryl and heteroaryl, wherein each of said R⁹ groups is optionally substituted one or more times;

R³⁰ is selected from alkyl and (C₀-C₆)-alkyl-aryl;

R^{a} and R^{b} are independently selected from hydrogen, CN, alkyl, haloalkyl, S(O)ₓNR¹⁰R¹¹, S(O)ₓR¹⁰ and C(O)NR¹⁰R¹¹, wherein alkyl and haloalkyl are optionally substituted one or more times;

E is selected from a bond, CR¹⁰R¹¹, O, NR⁵, S, S═O, S(═O)₂, C(═O), N(R¹⁰)(C═O), (C═O)N(R¹⁰), N(R¹⁰)S(═O)₂, S(═O)₂N(R¹⁰), C═N—OR¹¹, —C(R¹⁰R¹¹)C(R¹⁰R¹¹)—, —CH₂—W— and

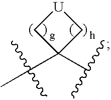

W is selected from O, NR⁵, S, S═O, S(═O)₂, N(R¹⁰)(C═O), N(R¹⁰)S(═O)₂ and S(═O)₂N(R¹⁰);

U is selected from C(R⁵R¹⁰), NR⁵, O, S, S═O and S(═O)₂;

Q is selected from 3-7 membered cycloalkyl, 4-7 membered heterocyclyl, 5-6 membered heteroaryl and 6-membered aryl;

A and B are independently selected from C, N, O and S;

L, M and T are independently selected from C and N;

g and h are independently selected from 0-2;

q is selected from 0-4;

r is selected from 0-1;

w is selected from 0-4;

x is selected from 0-2;

y is selected from 1 and 2;

z is selected from 0-2; and

wherein the dotted line represents optionally a double bond.

All remaining variables are as defined above.

More specifically, in some tricyclic embodiments R³ may be: 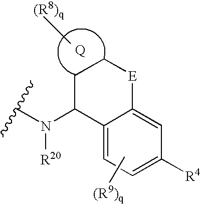
wherein:

E is selected from a bond, CR¹⁰R¹¹, O, NR⁵, S, S═O, S(═O)₂, C(═O), N(R¹⁰)(C═O), (C═O)N(R¹⁰), N(R¹⁰)S(═O)₂, S(═O)₂N(R¹⁰), C═N—OR¹¹, —C(R¹⁰R¹¹)C(R¹⁰R¹¹)— and

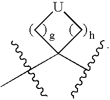

All remaining variables are as defined above.

In accordance with some embodiments of the present invention, one or more R⁴ groups may be heteroaryl. More specifically, in some embodiments R⁴ may be independently selected from: dioxole, imidazole, furan, thiazole, isothiazole, isoxazole, morpholine, 1,2,4-oxadiazole, 1,3,4-oxadiazole, 1,2,4-oxadiazole, 1,2-oxazine, 1,3-oxazine, 1,4-oxazine, oxirane, oxazole, 5-oxo-1,2,4-oxadiazole, 5-oxo-1,2,4-thiadiazole, piperzine, piperidine, pyran, pyrazine, pyrazole, pyridazine, pyridine, pyrimidine, pyrrole, pyrrolidine, tetrazine, tetrazole, thiazine, 1,2,3-thiadiazole, 1,2,4-thiadiazole, 1,3,4-thiadiazole, 1,2,5-thiadiazole, thiatriazole, 1,2-thiazine, 1,3-thiazine, 1,4-thiazine, thiazole, 5-thioxo-1,2,4-diazole, thiomorpholine, thiophene, thiopyran, 1,2,3-triazine, 1,2,4-triazine, 1,3,5-triazine, 1,2,4-triazole, 1,2,3-triazole or triazolones, which are optionally substituted.

In some embodiments of the present invention, R¹ may be:

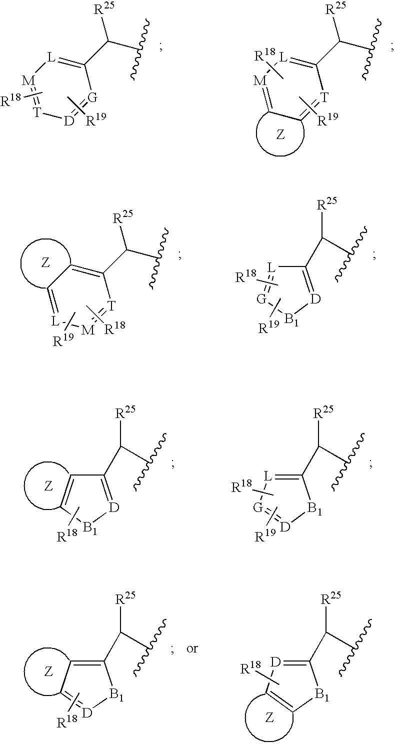

wherein:

R¹⁸ and R¹⁹ are independently selected from hydrogen, alkyl, haloalkyl, alkynyl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, alkynyl and haloalkyl are optionally substituted one or more times;

R²⁵ is selected from hydrogen, alkyl, cycloalkyl, C(O)NR¹⁰R¹¹ and haloalkyl, wherein alkyl, cycloalkyl, and haloalkyl are optionally substituted one or more times;

B₁ is selected from NR¹⁰, O and S;

D, G, L, M and T are independently selected from C and N; and

Z is a 5- to 6-membered ring selected from cycloalkyl, heterocycloalkyl, aryl, and heteroaryl, wherein cycloalkyl, heterocycloalkyl, aryl, and heteroaryl are optionally substituted one or more times.

All remaining variables are as defined above.

More specifically, R¹ may be, but is not limited to, the following:

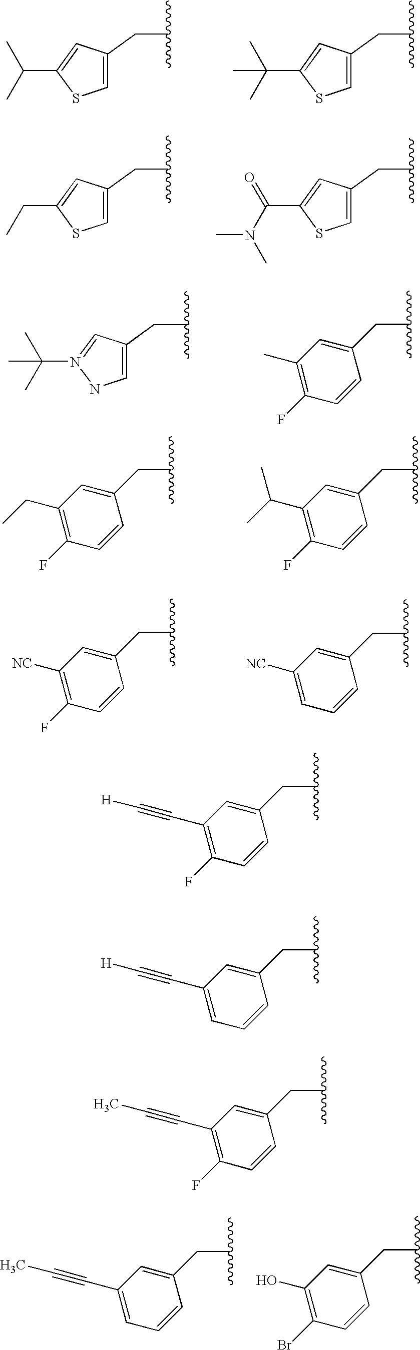

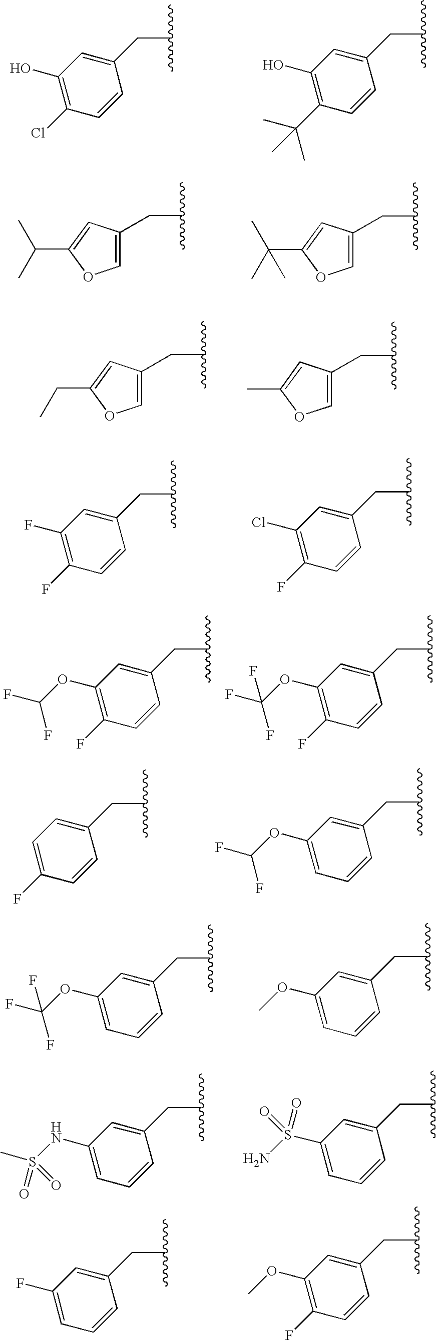

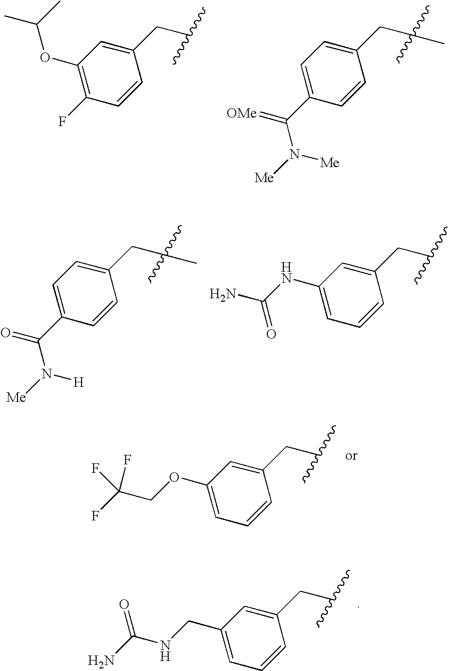

In some embodiments of the present invention, R¹ may include a bicyclic ring system. For instance, R¹ may be:

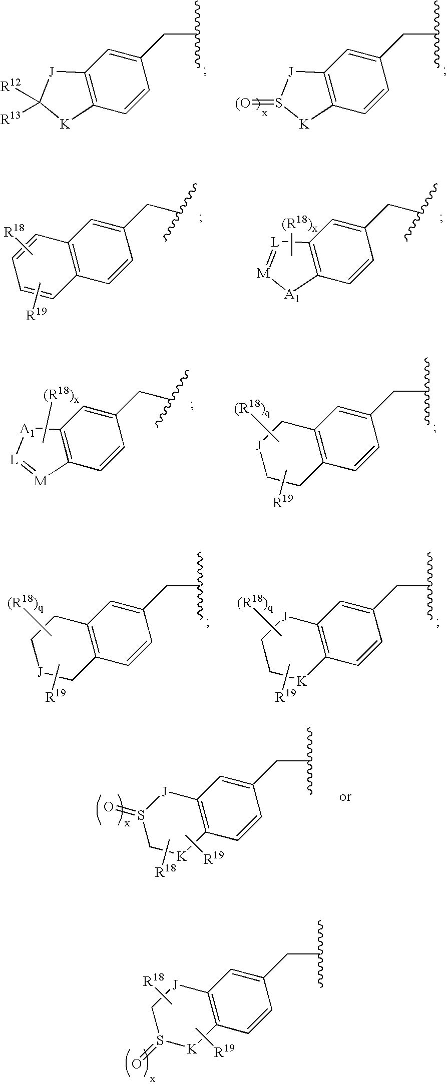

wherein:

R¹² and R¹³ are independently selected from hydrogen, alkyl and halo, wherein alkyl is optionally substituted one or more times, or optionally R¹² and R¹³ together form ═O, ═S or ═NR¹⁰;

R¹⁸ and R¹⁹ are independently selected from hydrogen, alkyl, haloalkyl, alkynyl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, alkynyl and haloalkyl are optionally substituted one or more times, or optionally two R¹⁸ groups together form ═O, ═S or ═NR¹⁰;

J and K are independently selected from CR¹⁰R¹¹, NR¹⁰, O and S(O)ₓ;

A₁ is selected from NR¹⁰, O, and S;

L and M are independently selected from C and N;

q is selected from 0-4; and

x is selected from 0-2.

All remaining variables are as defined above.

More specifically, R¹ may be, but is not limited to, the following:

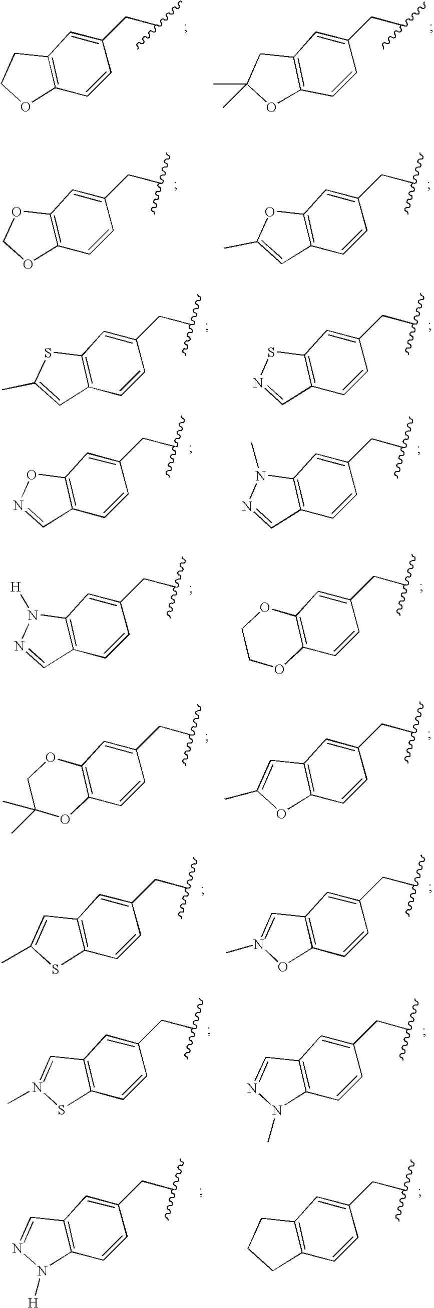

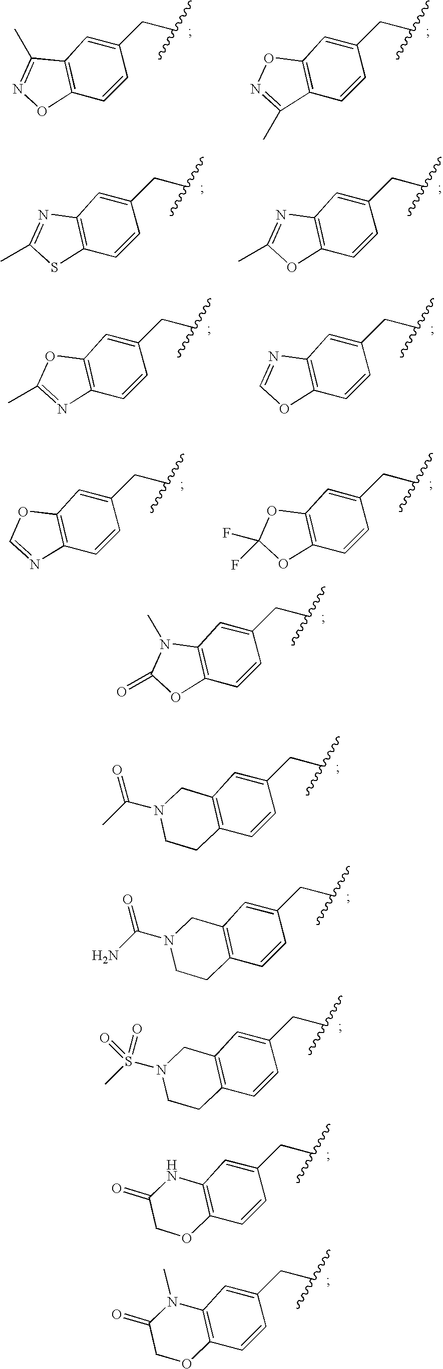

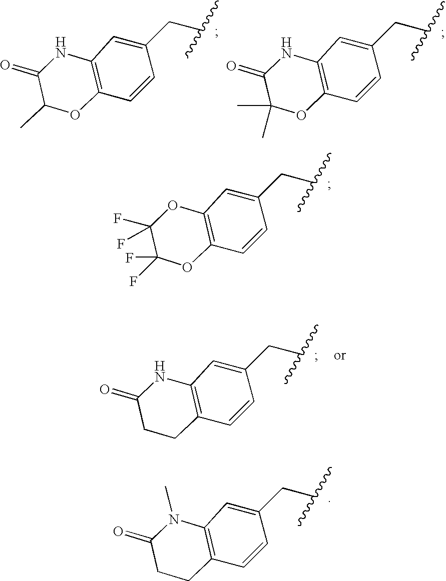

In some embodiments of the present invention, R¹ may be:

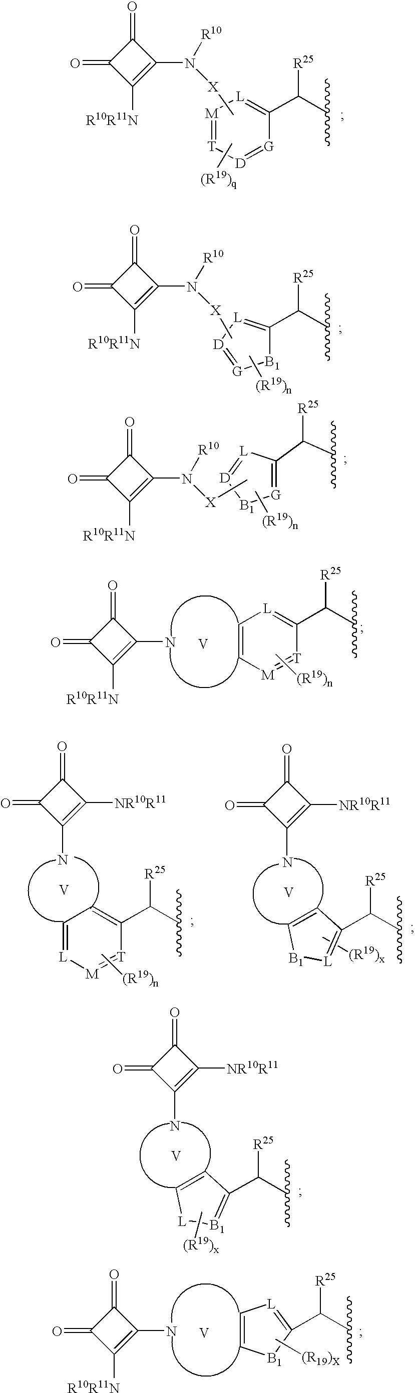

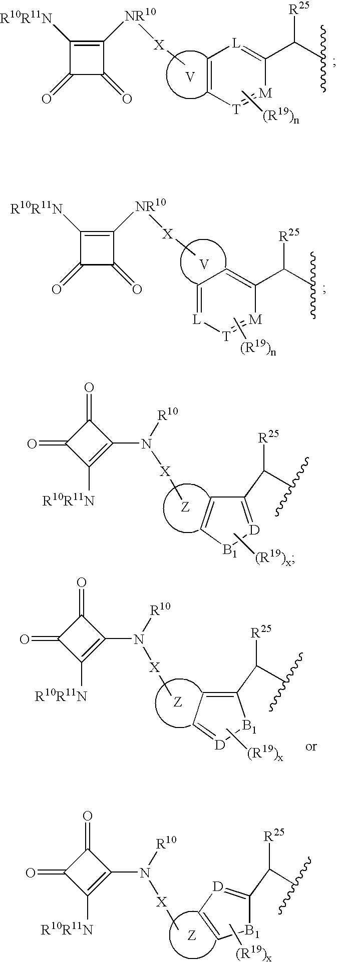

wherein:

R⁵ is selected from hydrogen, alkyl, C(O)NR¹⁰R¹¹, aryl, arylalkyl, SO₂NR¹⁰R¹¹, C(O)OR¹⁰ and CN, wherein alkyl, aryl and arylalkyl are optionally substituted one or more times;

R¹⁹ is selected from hydrogen, alkyl, haloalkyl, alkynyl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, alkynyl and haloalkyl are optionally substituted one or more times;

R²⁵ is selected from hydrogen, alkyl, cycloalkyl, C(O)NR¹⁰R¹¹ and haloalkyl, wherein alkyl, cycloalkyl, and haloalkyl are optionally substituted one or more times;

D, G, L, M and T are independently selected from C and N;

B, is selected from NR¹⁰, O and S;

X is selected from a bond and (CR¹⁰R¹¹)_{w}E(CR¹⁰R¹¹)_{w};

E is selected from a bond, CR¹⁰R¹¹, O, NR, S, S═O, S(═O)₂, C(═O), N(R¹⁰)(C═O), (C═O)N(R¹⁰), N(R¹⁰)S(═O)₂, S(═O)₂N(R¹⁰), C═N—OR¹¹, —C(R¹⁰R¹¹)C(R¹⁰R¹¹)—, —CH₂—W— and

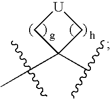

W is selected from O, NR⁵, S, S═O, S(═O)₂, N(R¹⁰)(C═O), N(R¹⁰)S(═O)₂ and S(═O)₂N(R¹⁰);

U is selected from C(R⁵R¹⁰), NR, O, S, S═O and S(═O)₂;

n is selected from 0-3;

q is selected from 0-4;

w is selected of 0-4;

x is selected from 0-2;

V is a 5- to 8-membered ring selected from cycloalkyl, heterocycloalkyl, aryl and heteroaryl, which is optionally substituted one or more times; and

Z is a 5- to 6-membered ring selected from cycloalkyl, heterocycloalkyl, aryl, and heteroaryl, wherein cycloalkyl, heterocycloalkyl, aryl, and heteroaryl are optionally substituted one or more times.

All remaining variables are as defined above.

More specifically, R¹ may be, but is not limited to, the following:

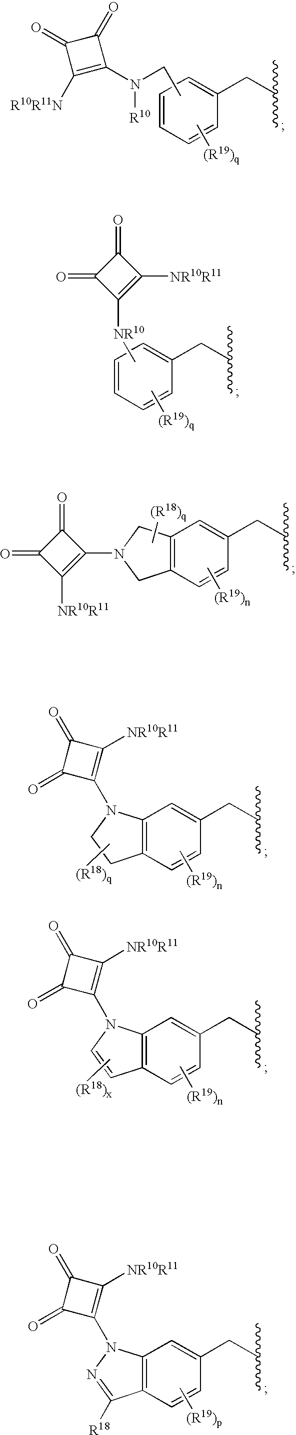

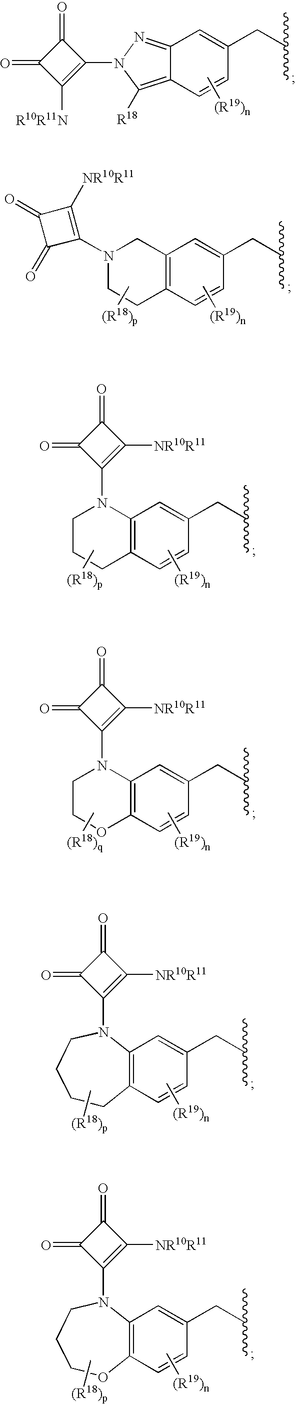

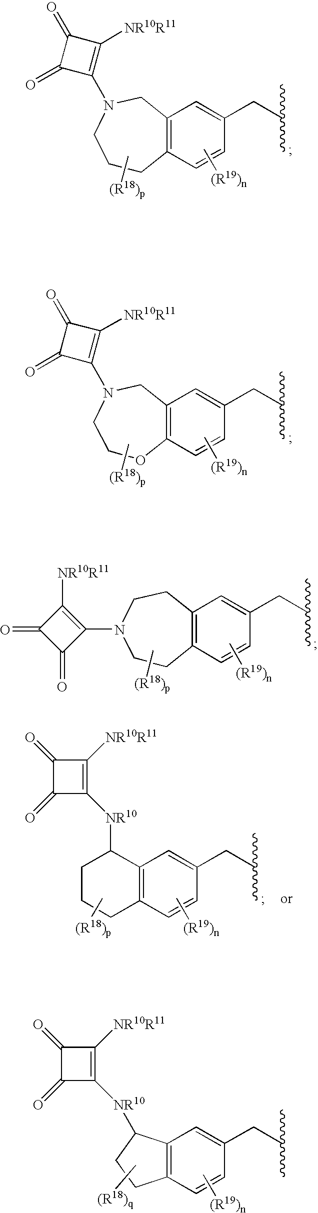

wherein:

R¹⁸ and R¹⁹ are independently selected from hydrogen, alkyl, haloalkyl, alkynyl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, alkynyl and haloalkyl are optionally substituted one or more times, or optionally two R¹⁸ groups together form ═O, ═S or ═NR¹⁰;

n is selected from 0-3;

p is selected from 0-6;

q is selected from 0-4; and

x is selected from 0-2.

All remaining variables ate as defined above.

More specifically, R¹ may be, but is not limited to, the following:

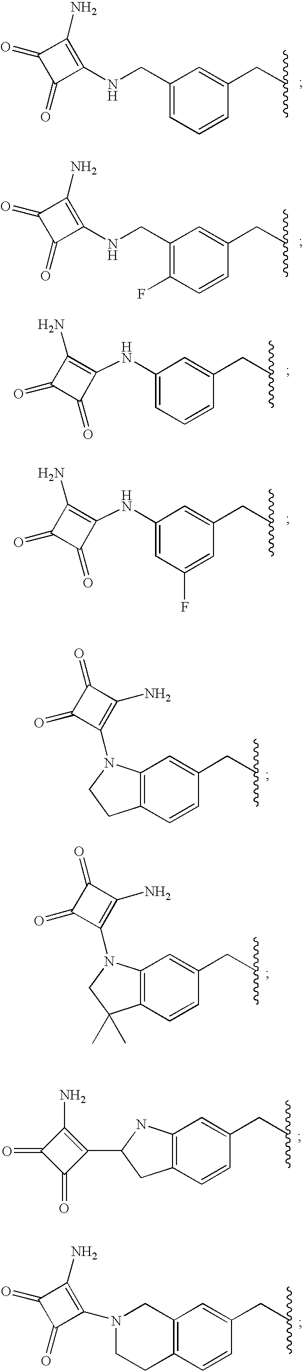

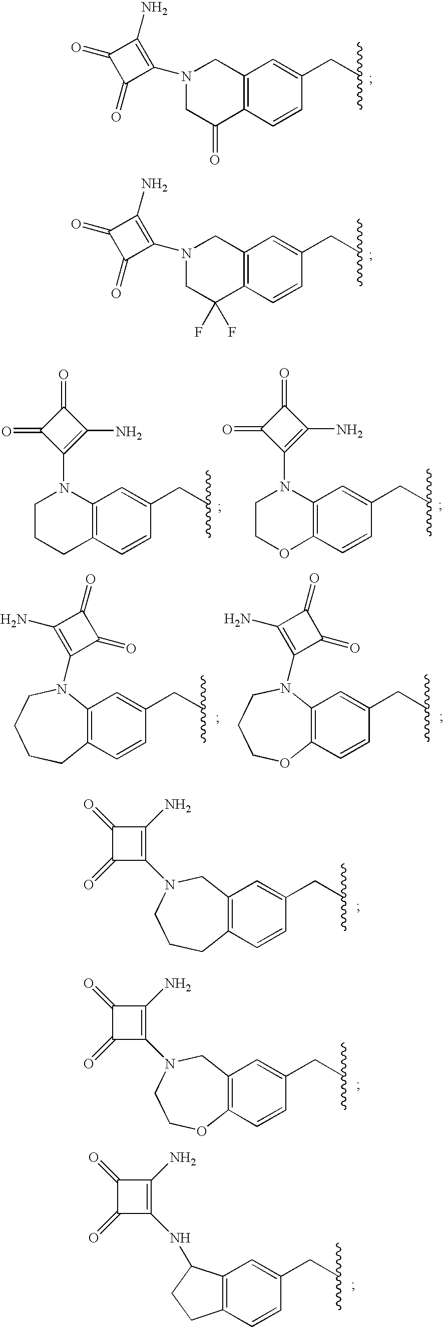

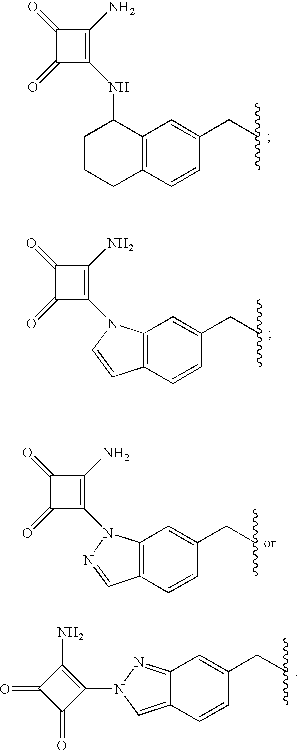

In accordance with some embodiments of the present invention, the multicyclic bis-amide MMP-13 inhibiting compounds of general Formula (I) may be represented by Formula (II):

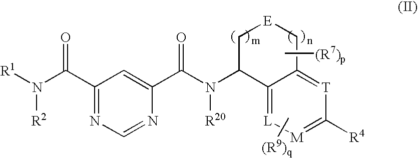

wherein:

R⁴ is selected from R¹⁰, hydrogen, alkyl, aryl, heteroaryl, halo, CF₃, COR¹⁰, OR¹⁰, NR¹⁰R¹¹, NO₂, CN, SO₂OR¹⁰, CO₂R¹⁰, C(O)NR¹⁰R¹¹, SO₂NR¹⁰R¹¹, SO₂R¹⁰, OC(O)R¹⁰, OC(O)NR¹⁰R¹¹, NR¹⁰C(O)R¹¹, NR¹⁰CO₂R , (C₀-C₆)-alkyl-C(═NR^{a})NHR^{b}, (C₀-C₆)-alkyl-NHC(═NR^{a})NHR^{b}, (C₀-C₆)-alkyl-C(O)OR¹⁰, (C₀-C₆)-alkyl-C(O)NR¹⁰R¹¹, (C₀-C₆)-alkyl-C(O)—NH—CN, O—(C₀-C₆)-alkyl-C(O)NR¹⁰R¹¹, S(O)ₓ—(C₀-C₆)-alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—SO₂NR¹⁰R¹¹, wherein each R⁴ group is optionally substituted by one or more R¹⁴ groups;

R⁵ is selected from hydrogen, alkyl, C(O)NR¹⁰R¹¹, aryl, arylalkyl, SO₂NR¹⁰R¹¹, C(O)OR¹⁰ and CN, wherein alkyl, aryl and arylalkyl are optionally substituted one or more times;

R⁷ is selected from hydrogen, alkyl, cycloalkyl, halo, R⁴ and NR¹⁰R¹¹, wherein alkyl and cycloalkyl are optionally substituted one or more times;

R⁹ is selected from hydrogen, alkyl, CH(CH₃)CO₂H, halo, (C₀-C₆)-alkyl-C(O)OR¹⁰, (C₀-C₆)-alkyl-C(O)NR¹⁰R¹¹, (C₀-C₆)-alkyl-C(O)NH—CN, O—(C₀-C₆)-alkyl-C(O)NR¹⁰R¹¹, S(O)_{y}-alkyl-C(O)OR¹⁰ , S(O)_{z}-alkyl-C(O)NR¹⁰R¹¹, (C₀-C₆)-alkyl-C(O)NR¹⁰—(C₀-C₆)-alkyl-NR¹⁰R¹¹, C(O)NR¹⁰—(C₀-C₆)-alkyl-heteroaryl, C(O)NR¹⁰—(C₀-C₆)-alkyl-aryl, CH₂NR¹⁰R¹¹, (CH₂)_{y}NR¹⁰C(O)-alkyl, (CH₂)_{w}NR¹⁰C(O)—(C₀-C₆)-alkyl-aryl, (CH₂)_{w}NR¹⁰C(O)—(C₀-C₆)-alkyl-heteroaryl, (CH₂)_{w}NR¹⁰C(O)O-alkyl, (CH₂)_{w}NR¹⁰C(O)O—(C₀-C₆)-alkyl-aryl, (CH₂)_{w}NR¹⁰C(O)O—(C₀-C₆)-alkyl-heteroaryl, (CH₂)_{w}NR¹⁰C(O)ONR¹⁰R¹¹, (CH₂)_{w}NR¹⁰S(O)₂—(C₀-C₆)-alkyl-aryl, (CH₂)_{w}NR¹⁰S(O)₂—(C₀-C₆)-alkyl-heteroaryl, (CH₂)_{w}NR¹⁰S(O)₂—NR¹⁰-alkyl, (CH₂)_{w}NR¹⁰S(O)₂NR¹⁰—(C₀-C₆)-alkyl-aryl, (CH₂)_{w}NR¹⁰S(O)₂NR¹⁰—(C₀-C₆)-alkyl-heteroaryl, (CH₂)_{w}NR¹⁰C(O)NR¹⁰-SO₂—R³⁰ S(O)₂NR¹⁰—(C₀-C₆)-alkyl-aryl, S(O)₂NR¹⁰—(C₀-C₆)-alkyl-heteroaryl, S(O)₂NR¹⁰-alkyl, S(O)₂—(C₀-C₆)-alkyl-aryl, S(O)₂—(C₀-C₆)-alkyl-heteroaryl, O-heteroaryl and heteroaryl, wherein each of said R⁹ groups is optionally substituted one or more times;

R¹⁴ is selected from hydrogen, alkyl, arylalkyl, cycloalkyl-alkyl, heteroarylalkyl, heterocyclylalkyl and halo, wherein alkyl, arylalkyl, cycloalkyl-alkyl, heteroarylalkyl and heterocyclylalkyl are optionally substituted one or more times;

R³⁰ is selected from alkyl and (C₀-C₆)-alkyl-aryl;

R^{a} and R^{b} are independently selected from hydrogen, CN, alkyl, haloalkyl, S(O)ₓNR¹⁰R¹¹, S(O)ₓR¹⁰ and C(O)NR¹⁰R¹¹, wherein alkyl and haloalkyl are optionally substituted one or more times;

E is selected from a bond, CR¹⁰R¹¹, O, NR⁵, S, S═O, S(═O)₂, C(═O), N(R¹⁰)(C═O), (C═O)N(R¹⁰), N(R¹⁰)S(═O)₂, S(═O)₂N(R¹⁰), C═N—OR¹¹, —C(R¹⁰R¹¹)C(R¹⁰R¹¹)—, —CH₂—W— and

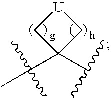

W is selected from O, NR⁵, S, S═O, S(═O)₂, N(R¹⁰)(C═O), N(R¹⁰)S(═O)₂ and S(═O)₂N(R¹⁰);

U is selected from C(R⁵R¹⁰), NR⁵, O, S, S═O and S(═O)₂;

L, M and T are independently selected from C and N;

g and h are independently selected from 0-2;

m and n are independently selected from 0-3, provided that:
(1) when E is present, m and n are not both 3;
(2) when E is —CH₂—W—, m and n are not 3; and
(3) when E is a bond, m and n are not 0;

p is selected from 0-6;

q is selected from 0-4;

w is selected from 0-4;

x is selected from 0-2;

y is selected from 1 and 2; and

z is selected from 0-2.

All remaining variables are as defined above.

In accordance with some embodiments of the present invention, the multicyclic bis-amide MMP-13 inhibiting compounds of general Formula (I) may be represented by Formula (III):

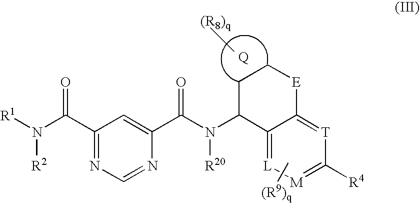

wherein:

R⁴ is selected from R¹⁰, hydrogen, alkyl, aryl, heteroaryl, halo, CF₃, COR¹⁰, OR¹⁰, NR⁰R¹¹, NO₂, CN, SO₂OR¹⁰, CO₂R¹⁰, C(O)NR¹⁰R¹¹, SO₂NR¹⁰R¹¹, SO₂R¹⁰, OC(O)R¹⁰, OC(O)NR¹⁰R¹¹, NR¹⁰C(O)R¹¹, NR¹⁰CO₂R¹¹, (C₀-C₆)-alkyl-C(═NR a)NHR b, (C₀-C₆)-alkyl- NHC(═NR^{a})NHR^{b}, (C₀-C₆)-alkyl-C(O)OR¹⁰, (C₀-C₆)-alkyl-C(O)NR¹⁰R¹¹, (C₀-C₆)-alkyl-C(O)- NH—CN, O—(C₀-C₆)-alkyl-C(O)NR¹⁰R¹¹, S(O),—(C₀-C₆)-alkyl-C(O)OR¹⁰, S(O),—(C₀-C₆)-alkyl- C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰R¹¹, (C₀-C₆)- alkyl-NR“-C(O)R¹⁰, (C₀-C₆)-alkyl-NR¹⁰-C(O)OR¹¹, (C₀-C₆)-alkyl-NR¹ —C(O)-NR¹⁰R¹¹, (C₀-C₆)- alkyl-NR¹⁰-SO₂NR¹⁰R¹¹, wherein each R⁴ group is optionally substituted by one or more RI⁴ groups;

R⁵ is selected from hydrogen, alkyl, C(O)NR¹⁰R¹¹, aryl, arylalkyl, SO₂NR⁰Rll, C(O)OR¹⁰ and CN, wherein alkyl, aryl and arylalkyl are optionally substituted one or more times;

R⁸ is selected from hydrogen, alkyl, OR¹⁰, NR¹⁰R¹¹, CN, arylalkyl, cycloalkyl-alkyl, heteroarylalkyl, heterocyclylalkyl and halo, wherein alkyl, arylalkyl, cycloalkyl-alkyl, heteroarylalkyl and heterocyclylalkyl are optionally substituted one or more times;

R⁹ is selected from hydrogen, alkyl, CH(CH₃)CO₂H, halo, (C₀-C₆)-alkyl-C(O)OR¹⁰, (C₀-C₆)-alkyl-C(O)NR¹⁰R¹¹, (C₀-C₆)-alkyl-C(O)NH—CN, O—(C₀-C₆)-alkyl-C(O)NR R¹¹, S(O)_{y}-alkyl-C(O)OR¹⁰, S(O)_{z}-alkyl-C(O)NR¹⁰R¹¹, (C₀-C₆)-alkyl-C(O)NR¹⁰—(C₀-C₆)-alkyl-NR¹⁰R¹¹, C(O)NR¹⁰—(C₀-C₆)-alkyl-heteroaryl, C(O)NR¹⁰—(C₀-C₆)-alkyl-aryl, CH₂NR¹⁰R¹¹, (CH₂)_{y}NR¹⁰C(O)-alkyl, (CH₂)_{w}NR¹⁰C(O)—(C₀-C₆)-alkyl-aryl, (CH₂),NR¹⁰C(O)—(C₀-C₆)-alkyl-heteroaryl, (CH₂)_{w}NR¹⁰C(O)O-alkyl, (CH₂)_{w}NR¹⁰C(O)O—(C₀-C₆)-alkyl-aryl, (CH₂)_{w}NR¹⁰C(O)O—(C₀-C₆)-alkyl-heteroaryl, (CH₂)_{w}NR¹⁰C(O)ONR¹⁰R¹¹, (CH₂)_{w}NR¹⁰S(O)₂—(C₀-C₆)-alkyl-aryl, (CH₂)_{w}NR¹⁰S(O)₂—(C₀-C₆)-alkyl-heteroaryl, (CH₂)_{w}NR¹⁰S(O)₂—NR¹⁰-alkyl, (CH₂)_{w}NR¹⁰S(O)₂NR¹⁰—(C₀-C₆)-alkyl-aryl, (CH₂)_{w}NR¹⁰S(O)₂NR¹⁰—(C₀-C₆)-alkyl-heteroaryl, (CH₂)_{w}NR¹⁰C(O)NR¹⁰—SO₂—R³⁰, S(O)₂NR¹⁰—(C₀-C₆)-alkyl-aryl, S(O)₂NR¹⁰—(C₀-C₆)-alkyl-heteroaryl, S(O)₂NR¹⁰-alkyl, S(O)₂—(C₀-C₆)-alkyl-aryl, S(O)₂—(C₀-C₆)-alkyl-heteroaryl, O-heteroaryl and heteroaryl, wherein each of said R⁹ groups is optionally substituted one or more times;

R¹⁴ is selected from hydrogen, alkyl, arylalkyl, cycloalkyl-alkyl, heteroarylalkyl, heterocyclylalkyl and halo, wherein alkyl, arylalkyl, cycloalkyl-alkyl, heteroarylalkyl and heterocyclylalkyl are optionally substituted one or more times;

R³⁰ is selected from alkyl and (C₀-C₆)-alkyl-aryl;

R^{a} and R^{b} are independently selected from hydrogen, CN, alkyl, haloalkyl, S(O)ₓNR¹⁰R¹¹, S(O)ₓR¹⁰ and C(O)NR¹⁰R¹¹, wherein alkyl and haloalkyl are optionally substituted one or more times;

E is selected from a bond, CR¹⁰R¹¹, O, NR⁵, S, S═O, S(═O)₂, C(═O), N(R¹⁰)(C═O), (C═O)N(R¹⁰), N(R¹⁰)S(═O)₂, S(═O)₂N(R¹⁰), C═N—OR¹¹, —C(R¹⁰R¹¹)C(R¹⁰R¹¹)—, —CH₂—W— and

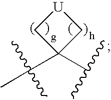

W is selected from O, NR⁵, S, S═O, S(═O)₂, N(R¹⁰)(C═O), N(R¹⁰)S(═O)₂ and S(═O)₂N(R¹⁰);

U is selected from C(R⁵R¹⁰), NR⁵, O, S, S═O and S(═O)₂;

Q is selected from 3-7 membered cycloalkyl, 4-7 membered heterocyclyl, 5-6 membered heteroaryl and 6-membered aryl;

L, M and T are independently selected from C and N;

q is selected from 0-4;

w is selected from 0-4;

x is selected from 0-2;

y is selected from 1 and 2; and

z is selected from 0-2.

All remaining variables are as defined above.

In addition, the multicyclic bis-amide MMP-13 inhibiting compounds may be represented by Formula (IV):

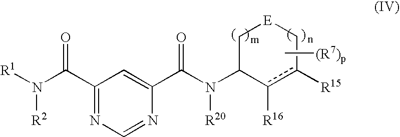

wherein:

R⁴ is selected from R¹⁰, hydrogen, alkyl, aryl, heteroaryl, halo, CF₃, COR¹⁰, OR¹⁰, NR¹⁰R¹¹, NO₂, CN, SO₂OR¹⁰, CO₂R¹⁰, C(O)NR¹⁰R¹¹, SO₂NR¹⁰R¹¹, SO₂R¹⁰, OC(O)R¹⁰, OC(O)NR¹⁰R¹¹, NR¹⁰C(O)R¹¹, NR¹⁰CO₂R¹¹, (C₀-C₆)-alkyl-C(═NR^{a})NHR^{b}, (C₀-C₆)-alkyl-NHC(═NR^{a})NHR^{b}, (C₀-C₆)-alkyl-C(O)OR¹⁰, (C₀-C₆)-alkyl-C(O)NR¹⁰R¹¹, (C₀-C₆)-alkyl-C(O)—NH—CN, O—(C₀-C₆)-alkyl-C(O)NR¹⁰R¹¹, S(O)ₓ—(C₀-C₆)-alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰R¹¹, (C₀-C₆)-alkyl-NR¹⁰-C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—SO₂NR¹⁰R¹¹1, wherein each R⁴ group is optionally substituted by one or more R¹⁴ groups;

R⁵ is selected from hydrogen, alkyl, C(O)NR¹⁰R¹¹, aryl, arylalkyl, SO₂NR¹⁰R¹¹, C(O)OR¹⁰ and CN, wherein alkyl, aryl and arylalkyl are optionally substituted one or more times;

R⁷ is selected from hydrogen, alkyl, cycloalkyl, halo, R⁴ and NR¹⁰R¹¹, wherein alkyl and cycloalkyl are optionally substituted one or more times;

R¹⁴ is selected from hydrogen, alkyl, arylalkyl, cycloalkyl-alkyl, heteroarylalkyl, heterocyclylalkyl and halo, wherein alkyl, arylalkyl, cycloalkyl-alkyl, heteroarylalkyl and heterocyclylalkyl are optionally substituted one or more times;

R¹⁵ and R¹⁶ when taken together with the carbon atoms to which they are bound, form a ring selected from 6-membered aryl ring, 5- or 6-membered heteroaryl ring, 5- to 8-membered cycloalkyl ring, 5- to 8-membered heterocyclyl ring, 5- to 8-membered cycloalkenyl ring and 5- to 8-membered heterocycloalkenyl ring, wherein said ring is optionally substituted by one or more R⁴ groups;

R^{a} and R^{b} are independently selected from hydrogen, CN, alkyl, haloalkyl, S(O),NR¹⁰R¹¹, S(O)ₓR¹⁰ and C(O)NR¹⁰R¹¹, wherein alkyl and haloalkyl are optionally substituted one or more times;

E is selected from a bond, CR¹⁰R¹¹, O, NR⁵, S, S═O, S(═O)₂, C(═O), N(R¹⁰)(C═O), (C═O)N(R¹⁰), N(R¹⁰)S(═O)₂, S(═O)₂N(R¹⁰), C═N—OR¹¹, —C(R¹⁰R¹¹)C(R¹⁰R¹¹)—, —CH₂—W— and

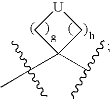

W is selected from O, NR⁵, S, S═O, S(═O)₂, N(R¹⁰)(C═O), N(R¹⁰)S(═O)₂ and S(═O)₂N(R¹⁰);

U is selected from C(R⁵R¹⁰), NR⁵, O, S, S═O and S(═O)₂;

g and h are independently selected from 0-2;

m and n are independently selected from 0-3, provided that:
(1) when E is present, m and n are not both 3;
(2) when E is —CH₂—W—, m and n are not 3; and
(3) when E is a bond, m and n are not 0;

p is selected from 0-6;

x is selected from 0-2; and

wherein the dotted line represents optionally a double bond.

All remaining variables are as defined above.

In addition, the multicyclic bis-amide MMP-13 inhibiting compounds of general Formula (I) may be represented by Formula (V):

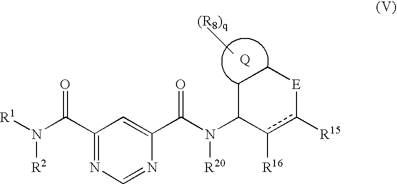

wherein:

R⁴ is selected from R¹⁰, hydrogen, alkyl, aryl, heteroaryl, halo, CF₃, COR¹⁰, OR¹⁰, NR¹⁰R¹¹, NO₂, CN, SO₂OR¹⁰, CO₂R¹⁰, C(O)NR¹⁰R¹¹, SO₂NR¹⁰R¹¹, SO₂R¹⁰, OC(O)R¹⁰, OC(O)NR¹⁰R¹¹, NR¹⁰C(O)R¹¹, NR¹⁰CO₂R¹¹, (C₀-C₆)-alkyl-C(═NR^{a})NHR^{b}, (C₀-C₆)-alkyl-NHC(═NR^{a})NHR^{b}, (C₀-C₆)-alkyl-C(O)OR¹⁰, (C₀-C₆)-alkyl-C(O)NR¹⁰R¹¹, (C₀-C₆)-alkyl-C(O)—NH—CN, O—(C₀-C₆)-alkyl-C(O)NR¹⁰R¹¹, S(O),—(C₀-C₆)-alkyl-C(O)OR¹⁰, S(O),—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—SO₂NR¹⁰R¹¹, wherein each R⁴ group is optionally substituted by one or more R¹⁴ groups;

R⁵ is selected from hydrogen, alkyl, C(O)NR¹⁰R¹¹, aryl, arylalkyl, SO₂NR¹⁰R¹¹, C(O)OR¹⁰, and CN, wherein alkyl, aryl and arylalkyl are optionally substituted one or more times;

R⁸ is selected from hydrogen, alkyl, OR¹⁰, NR¹⁰R¹¹, CN, arylalkyl, cycloalkyl-alkyl, heteroarylalkyl, heterocyclylalkyl and halo, wherein alkyl, arylalkyl, cycloalkyl-alkyl, heteroarylalkyl and heterocyclylalkyl are optionally substituted one or more times;

R¹⁴ is selected from hydrogen, alkyl, arylalkyl, cycloalkyl-alkyl, heteroarylalkyl, heterocyclylalkyl and halo, wherein alkyl, arylalkyl, cycloalkyl-alkyl, heteroarylalkyl and heterocyclylalkyl are optionally substituted one or more times;

R¹⁵ and R¹⁶ when taken together with the carbon atoms to which they are bound, form a ring selected from 6-membered aryl ring, 5- or 6-membered heteroaryl ring, 5- to 8-membered cycloalkyl ring, 5- to 8-membered heterocyclyl ring, 5- to 8-membered cycloalkenyl ring and 5- to 8-membered heterocycloalkenyl ring, wherein said ring is optionally substituted by one or more R⁴ groups;

R^{a} and R^{b} are independently selected from hydrogen, CN, alkyl, haloalkyl, S(O)ₓNR¹⁰R¹¹, S(O)ₓR¹⁰ and C(O)NR¹⁰R¹¹, wherein alkyl and haloalkyl are optionally substituted one or more times;

E is selected from a bond, CR¹⁰R¹¹, O, NR⁵, S, S═O, S(═O)₂, C(═O), N(R¹⁰)(C═O), (C═O)N(R¹⁰), N(R¹⁰)S(═O)₂, S(═O)₂N(R¹⁰), C═N-OR¹¹, —C(R¹⁰R¹¹)C(R¹⁰R¹¹)—, —CH₂—W— and

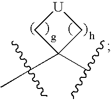

W is selected from O, NR⁵, S, S═O, S(═O)₂, N(R¹⁰)(C═O), N(R¹⁰)S(═O)₂ and S(═O)₂N(R¹⁰);

U is selected from C(⁵R¹⁰), NR⁵, O, S, S═O and S(═O)₂;

Q is selected from 3-7 membered cycloalkyl, 4-7 membered heterocyclyl, 5-6 membered heteroaryl and 6 membered aryl;

g and h are independently selected from 0-2;

x is selected from 0-2; and

wherein the dotted line represents optionally a double bond.

All remaining variables are as defined above.

More specifically, the compounds of Formula (I) may be selected from, but are not limited to, the following:

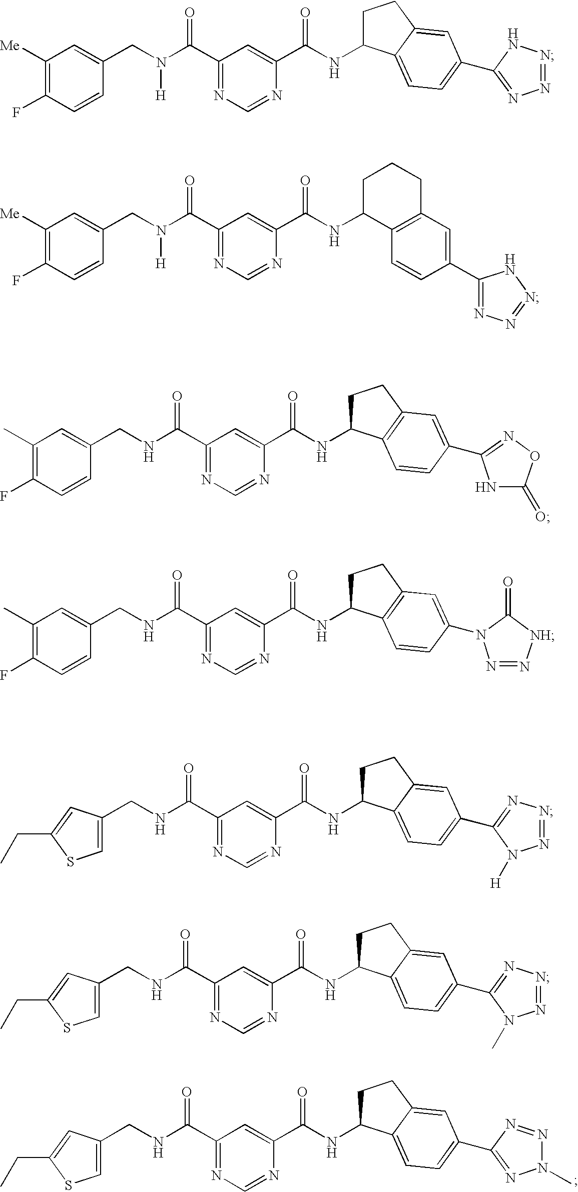

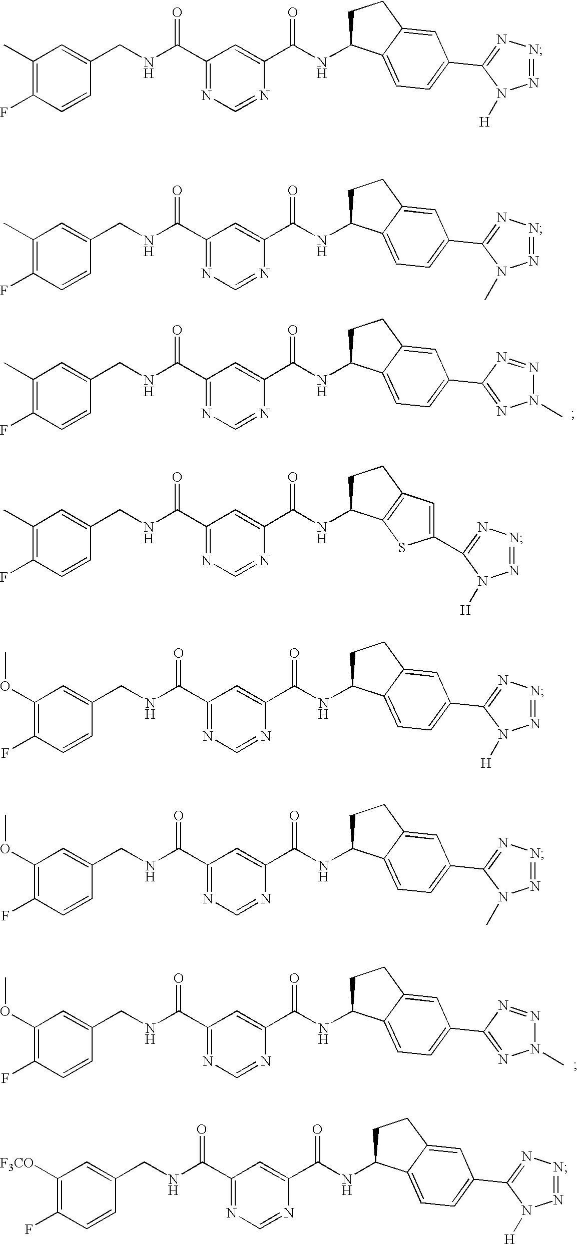

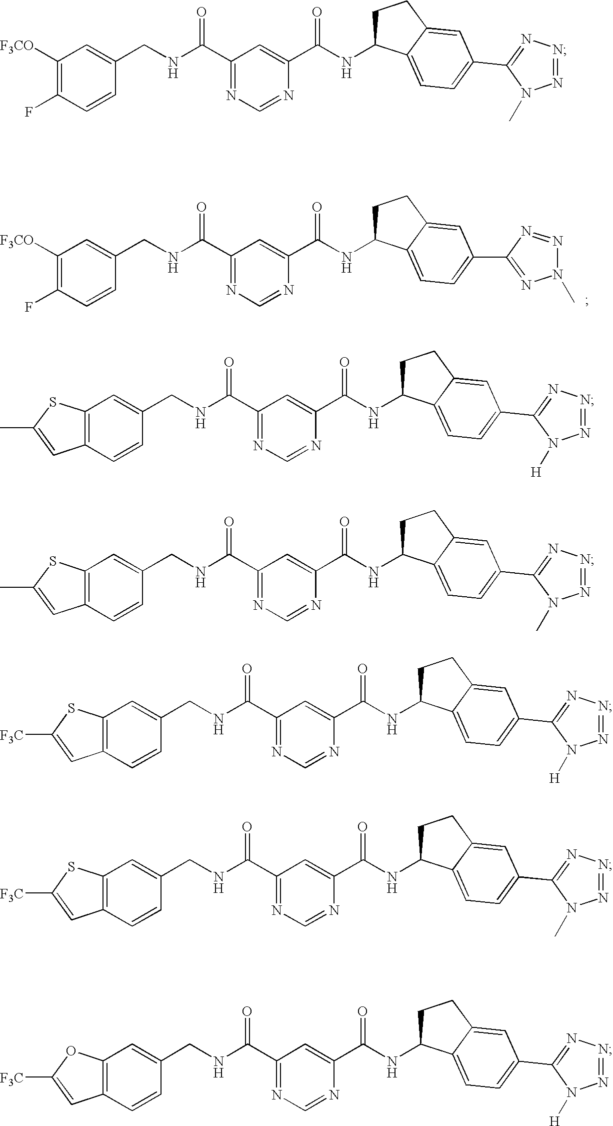

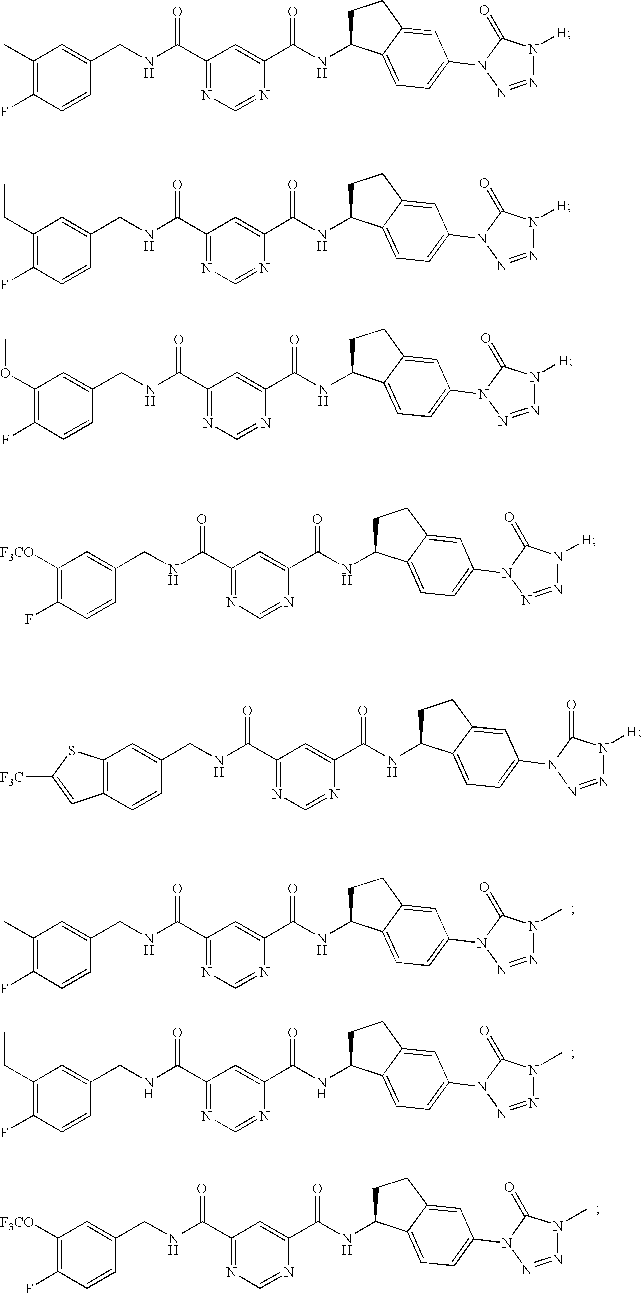

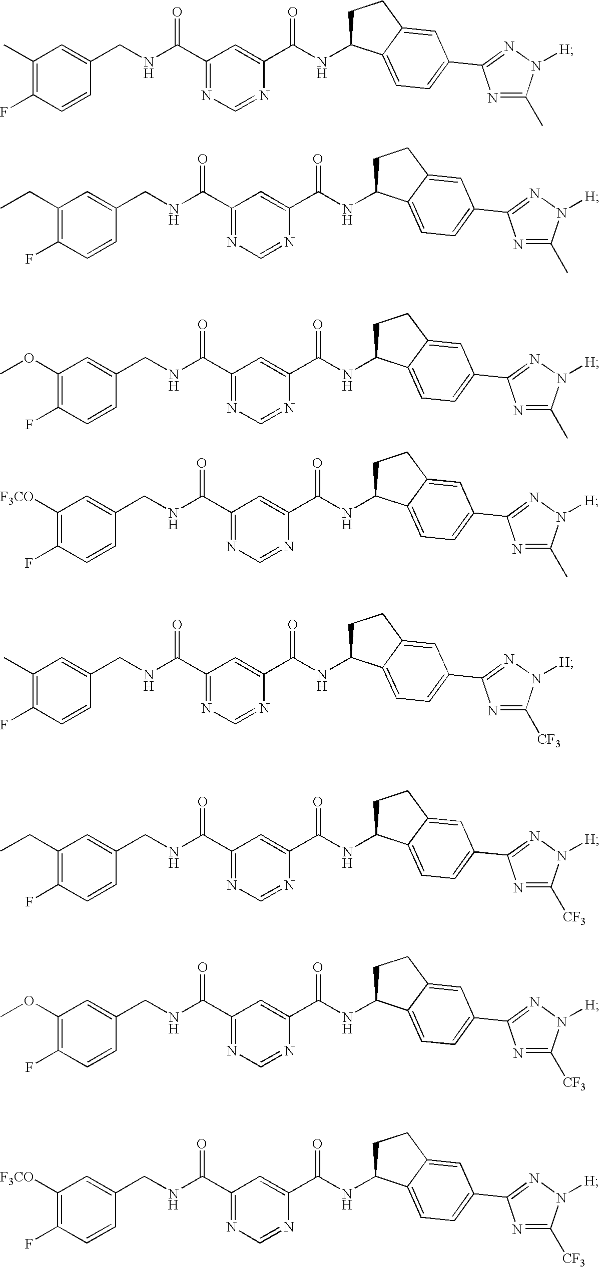

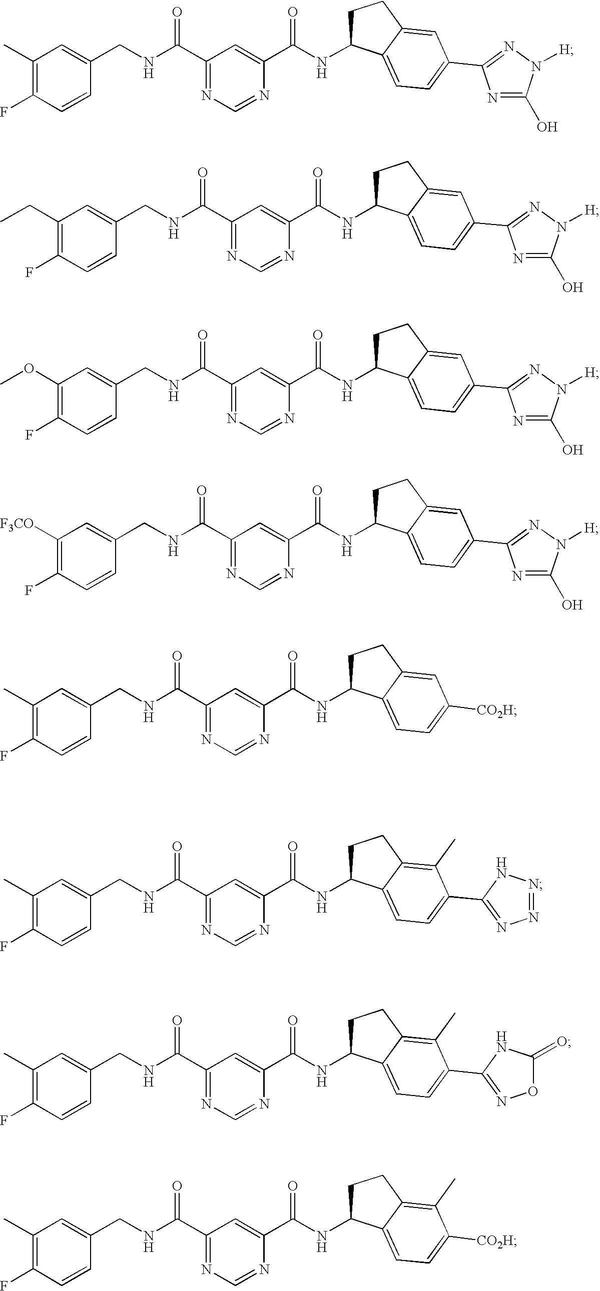

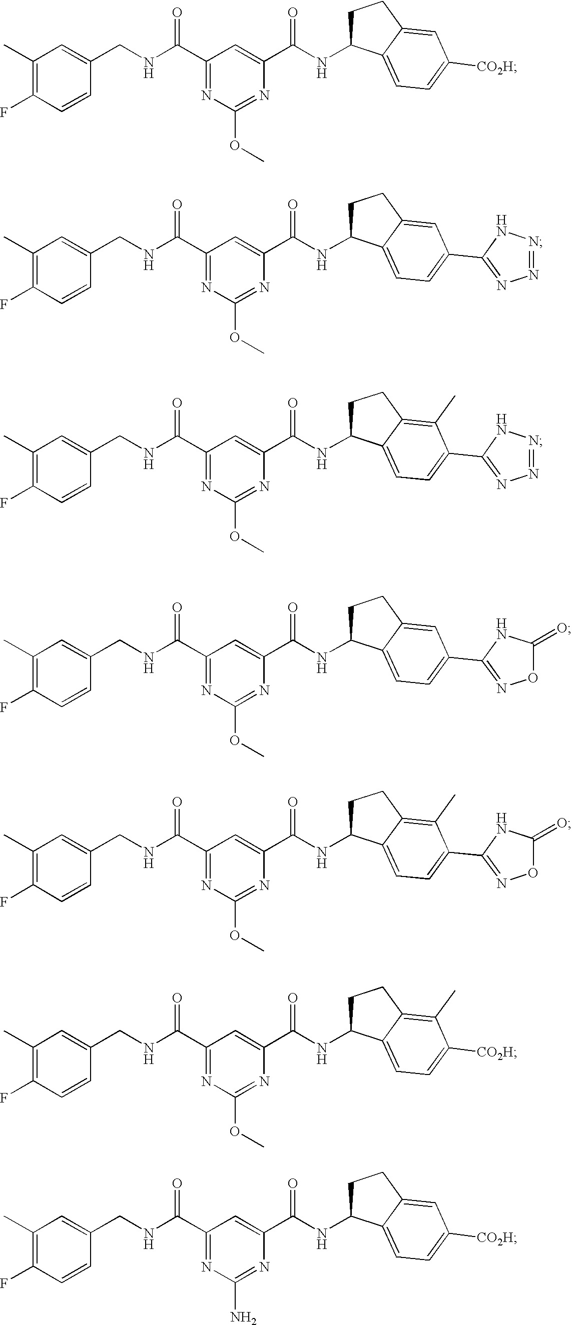

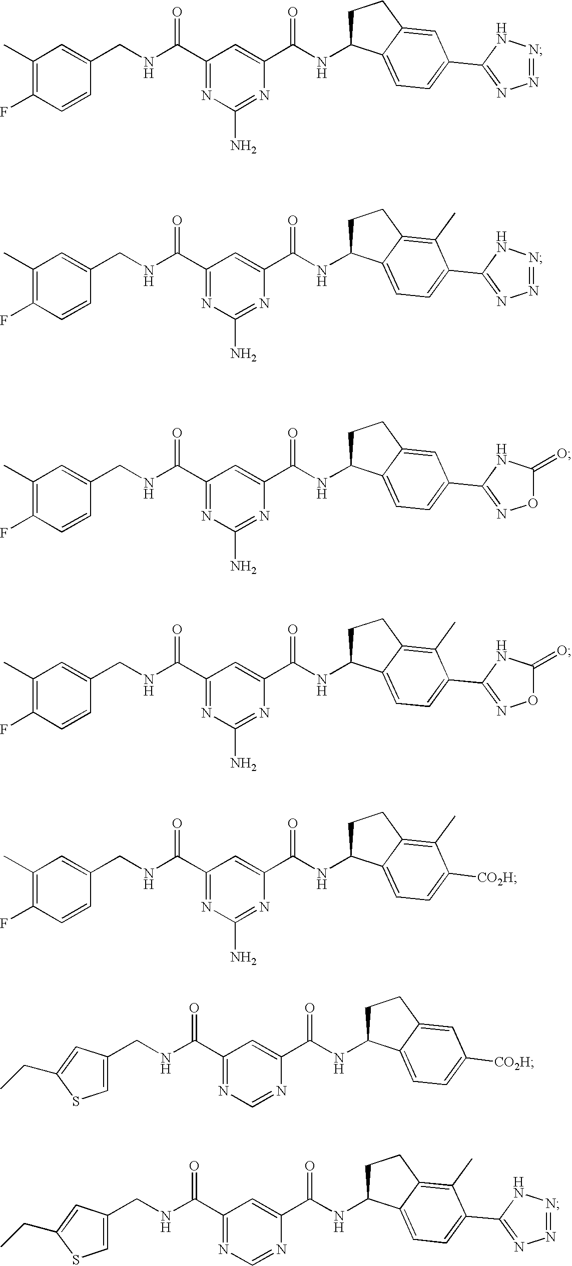

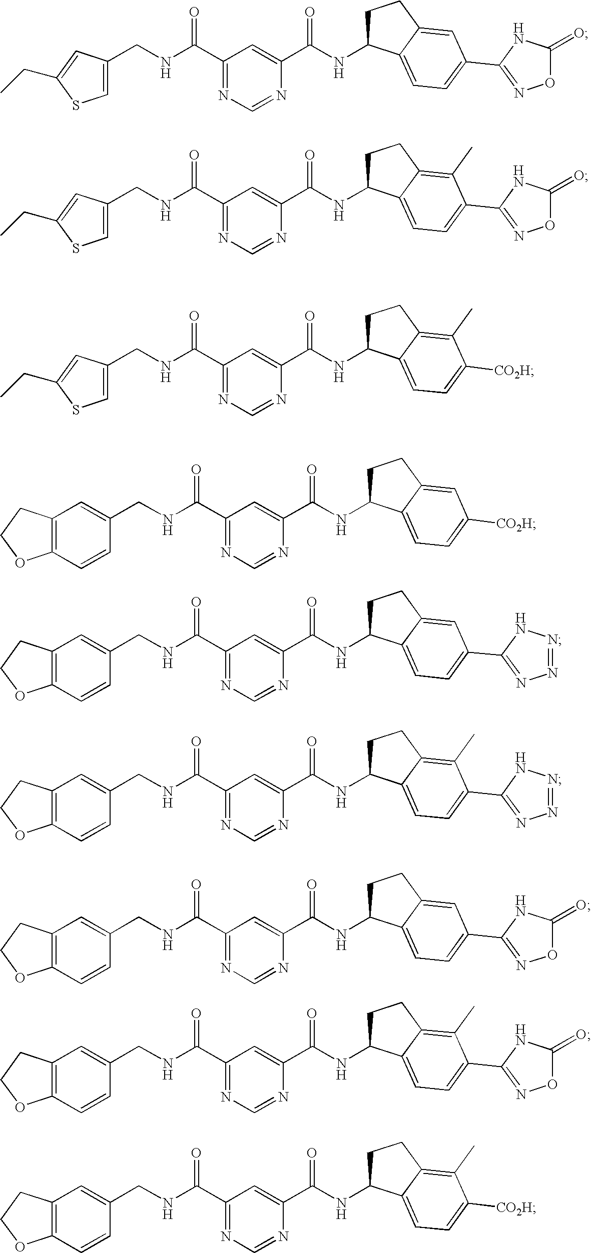

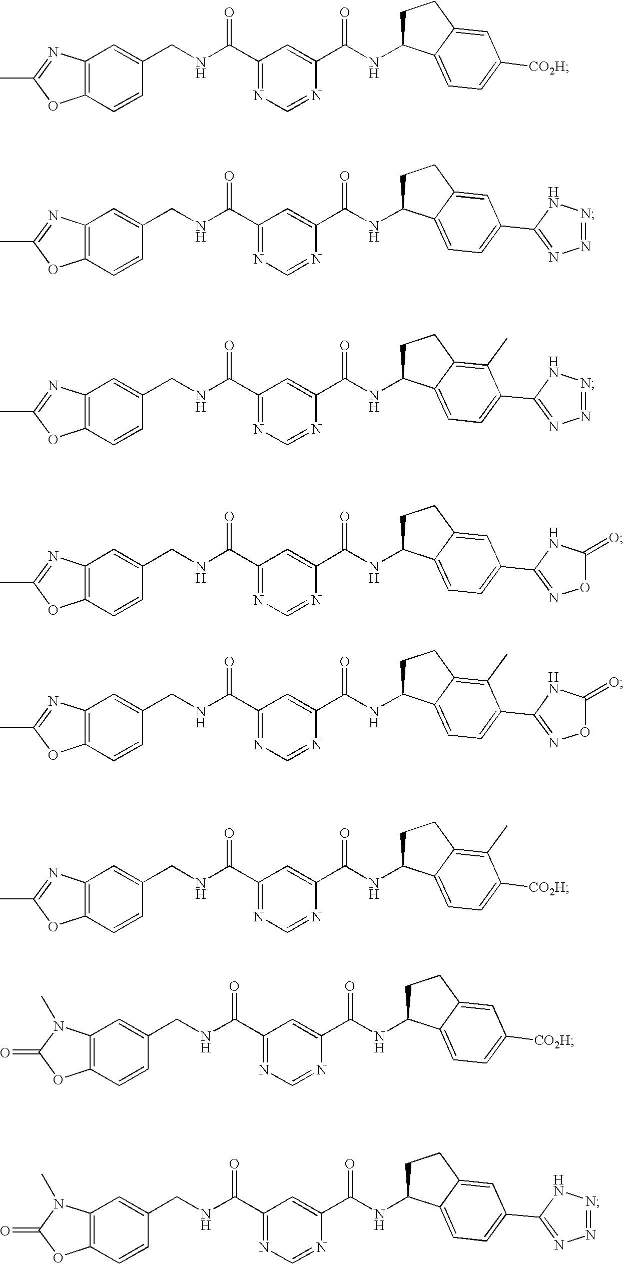

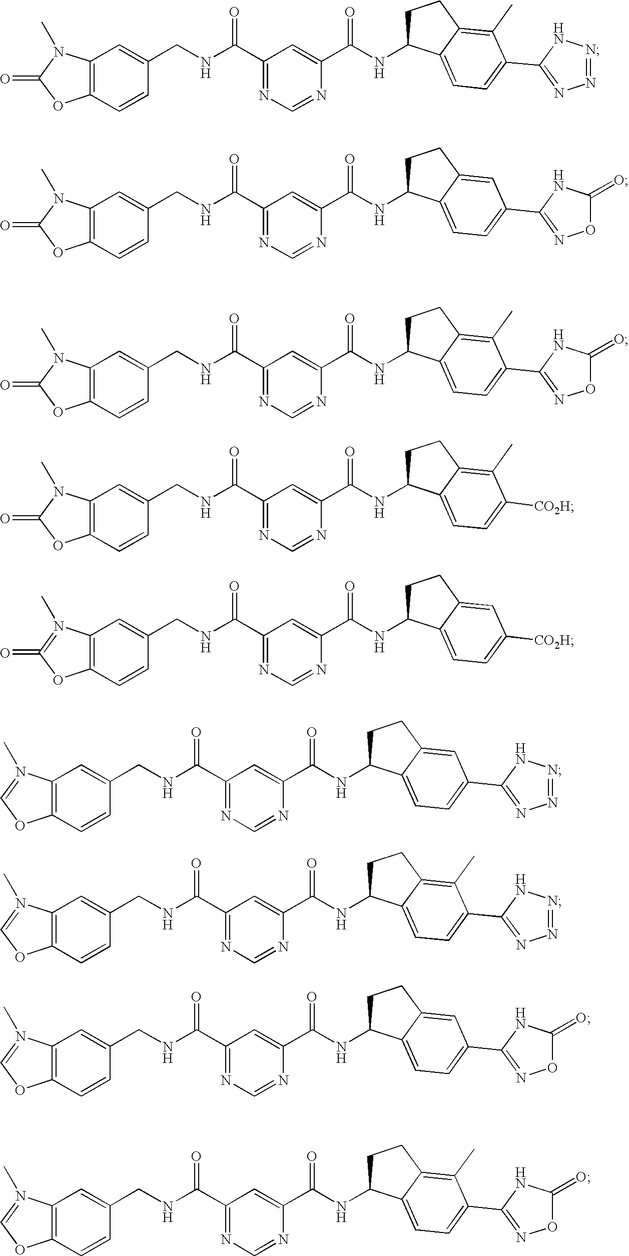

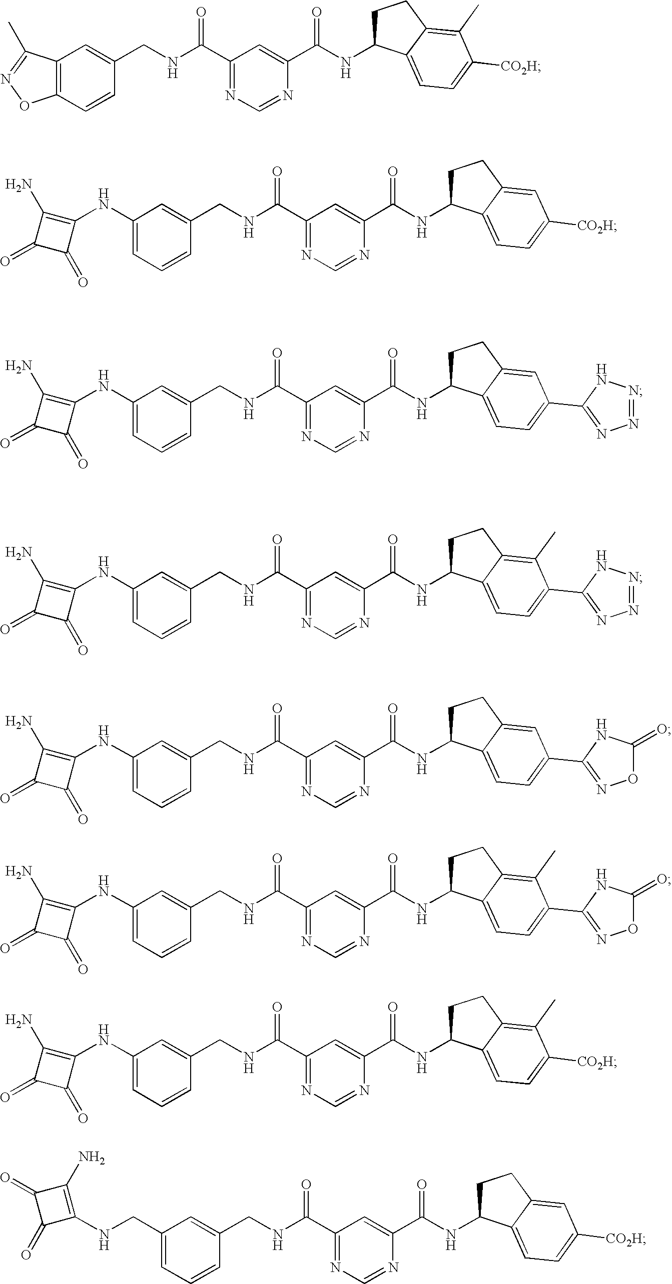

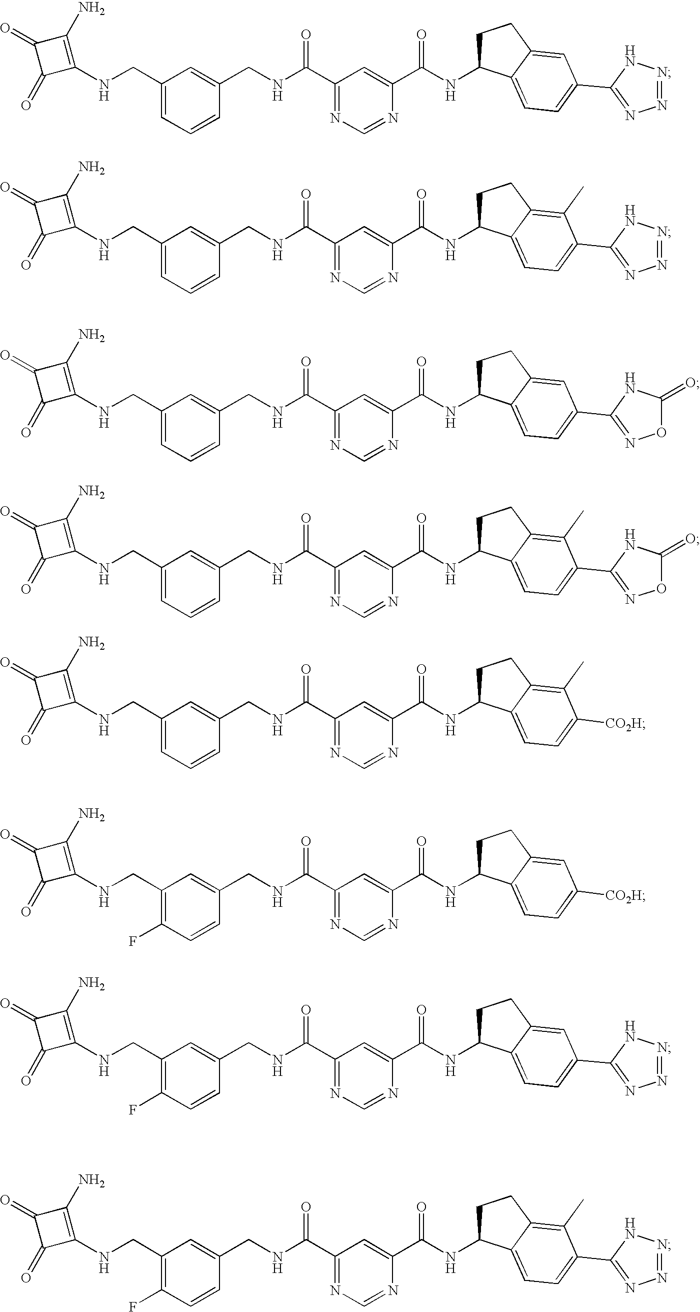

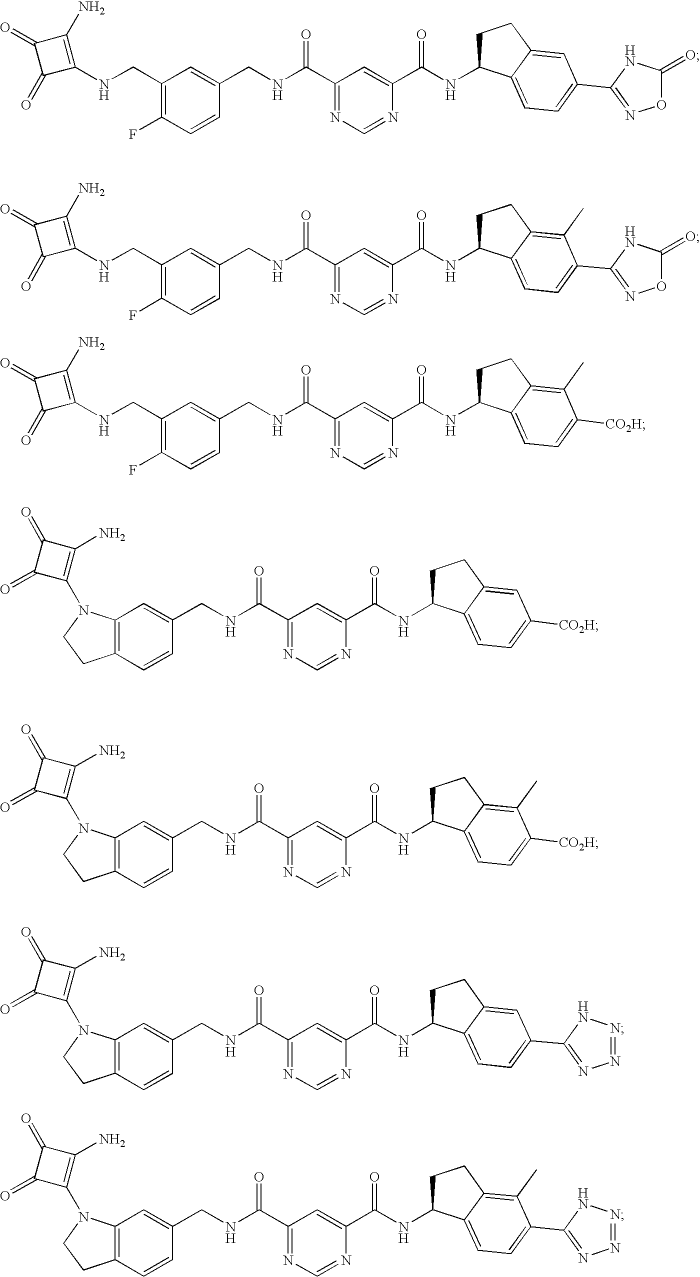

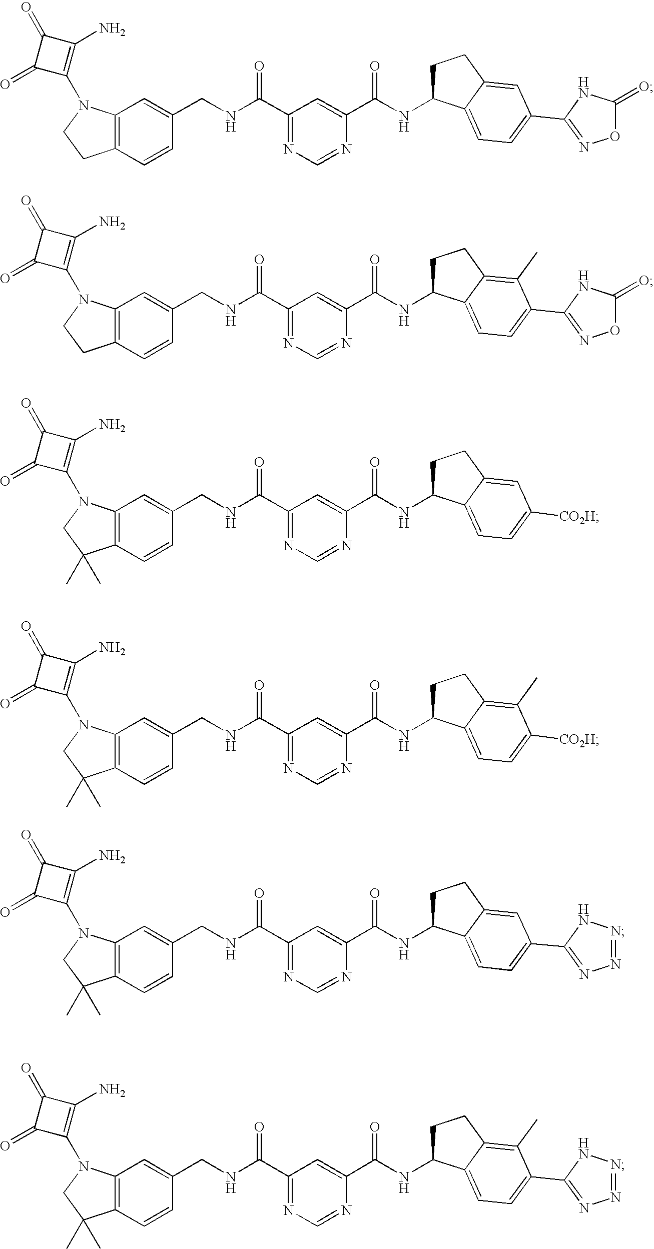

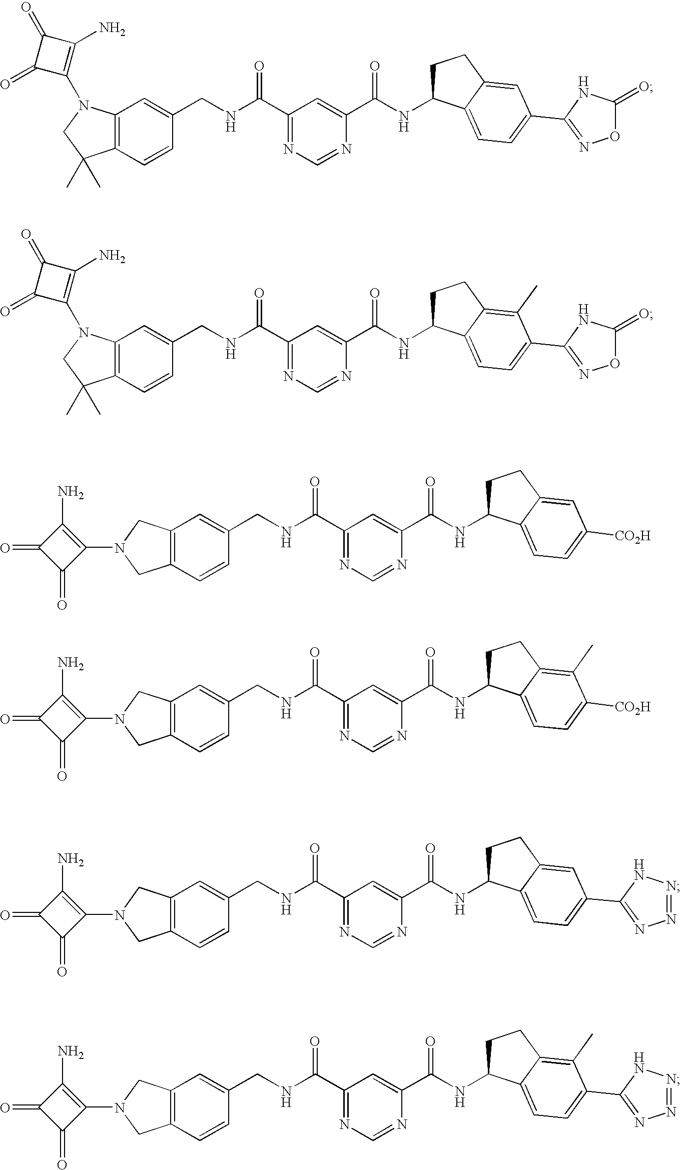

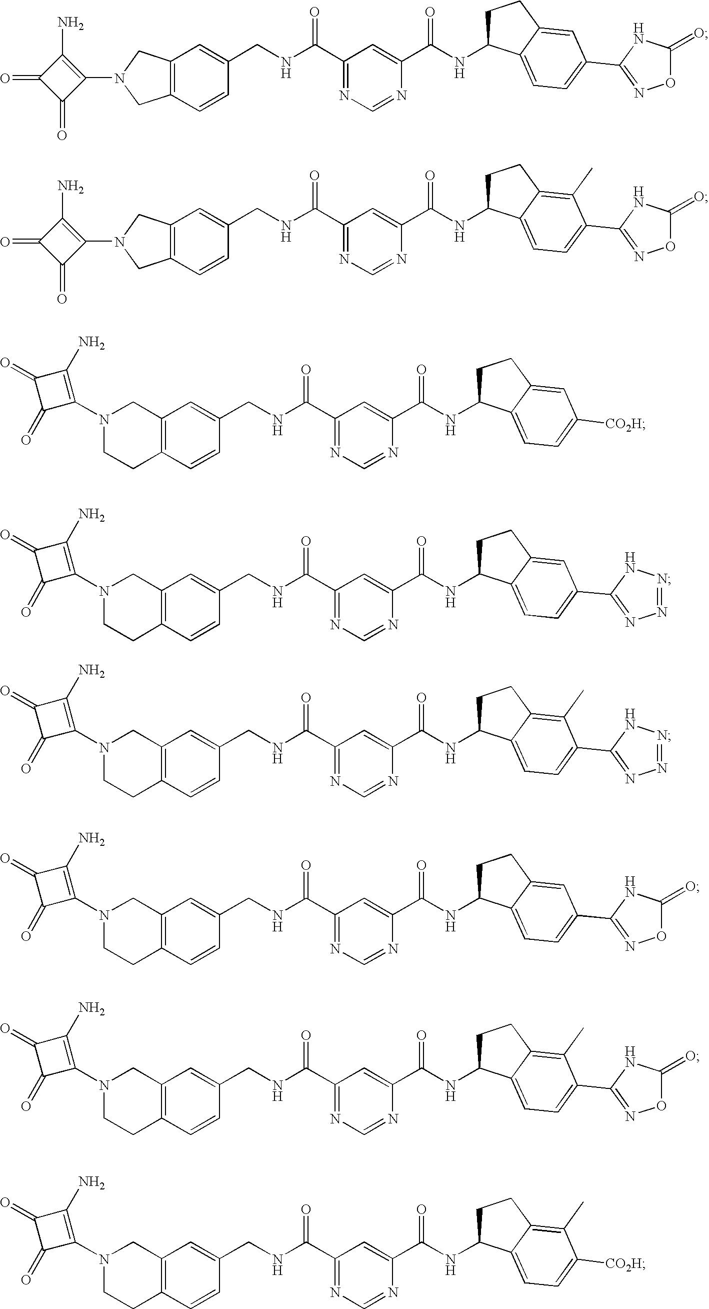

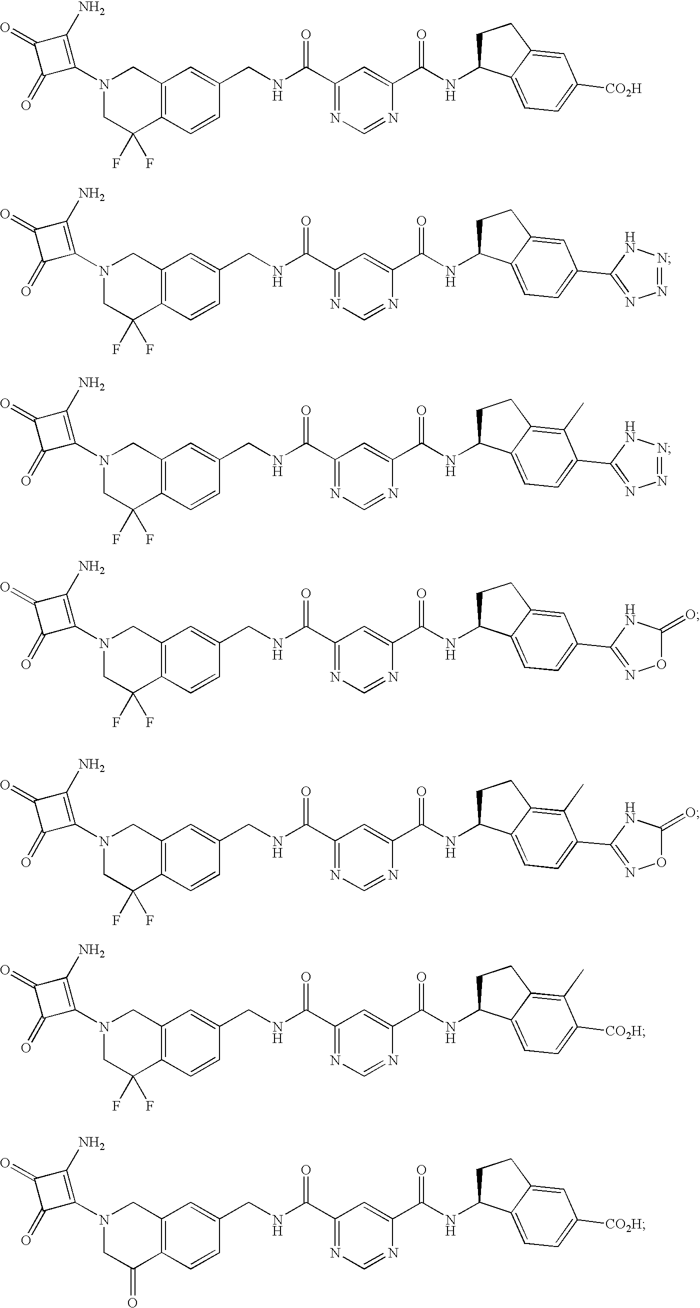

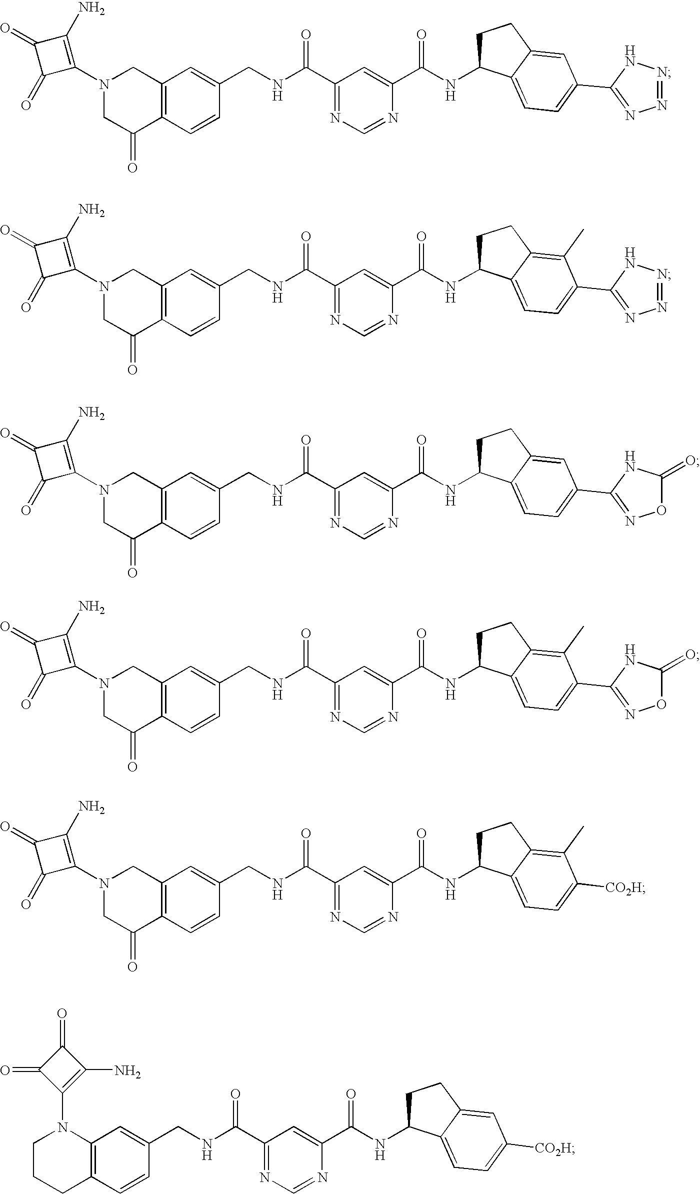

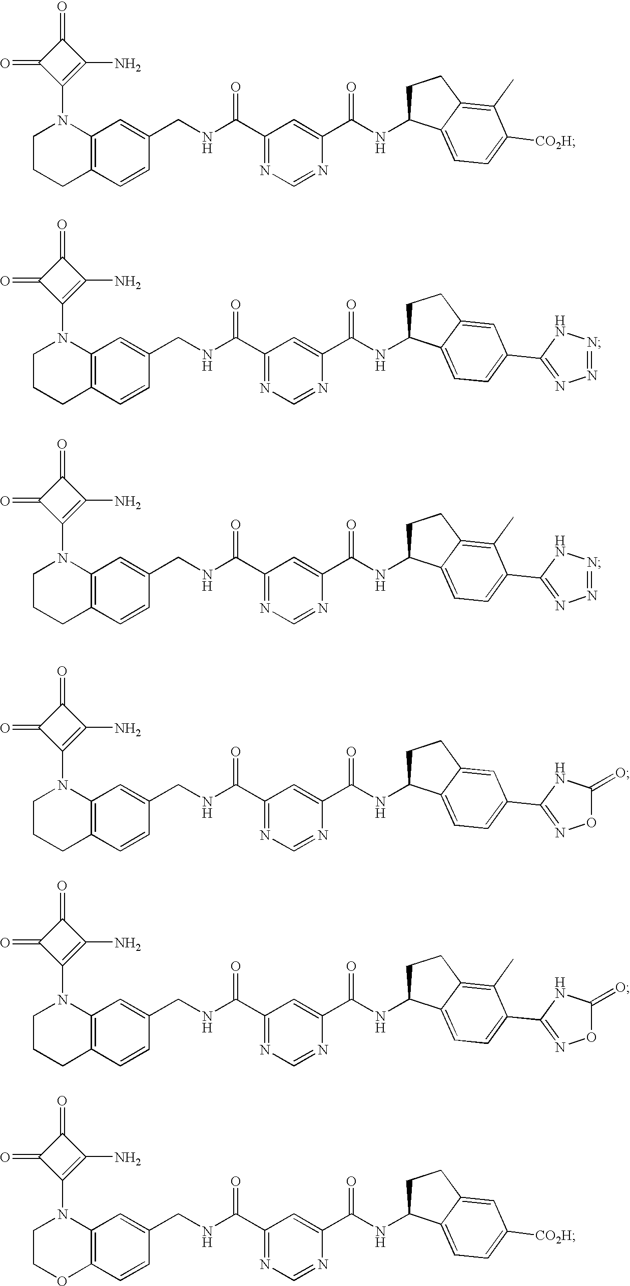

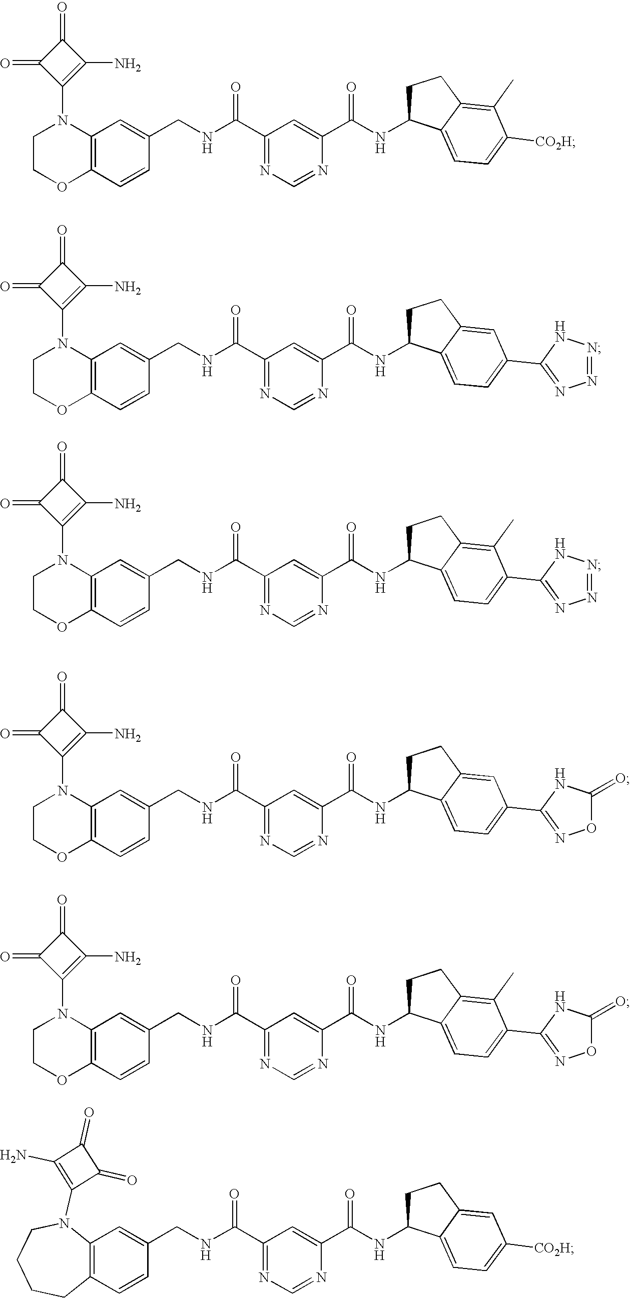

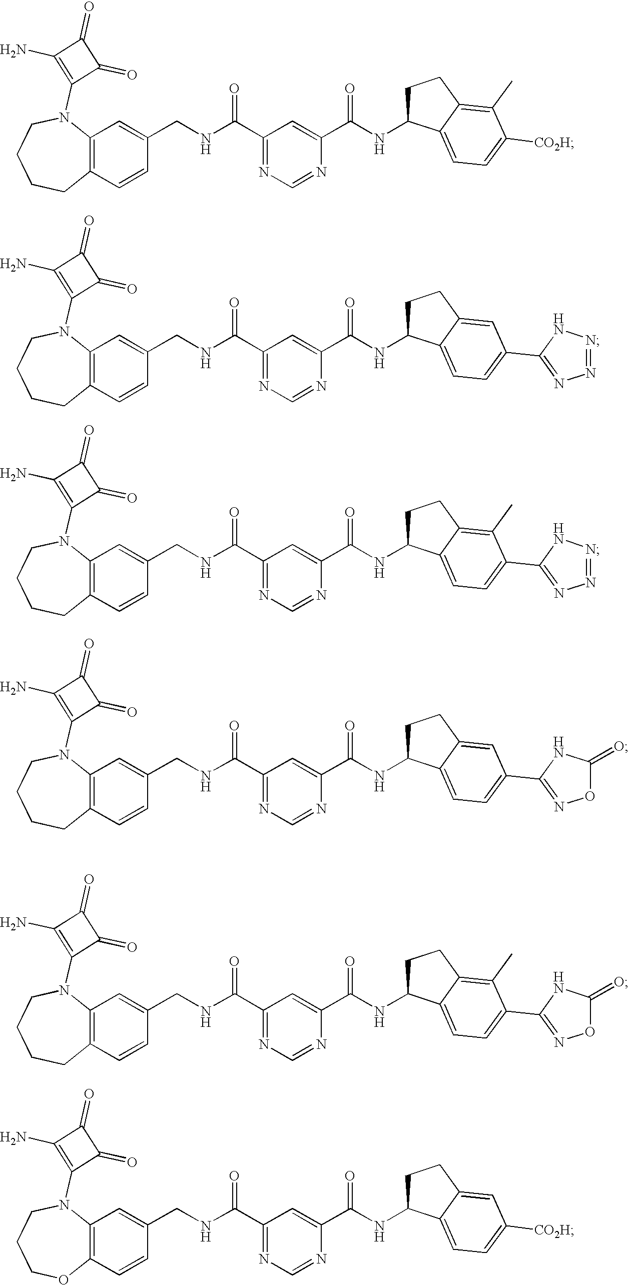

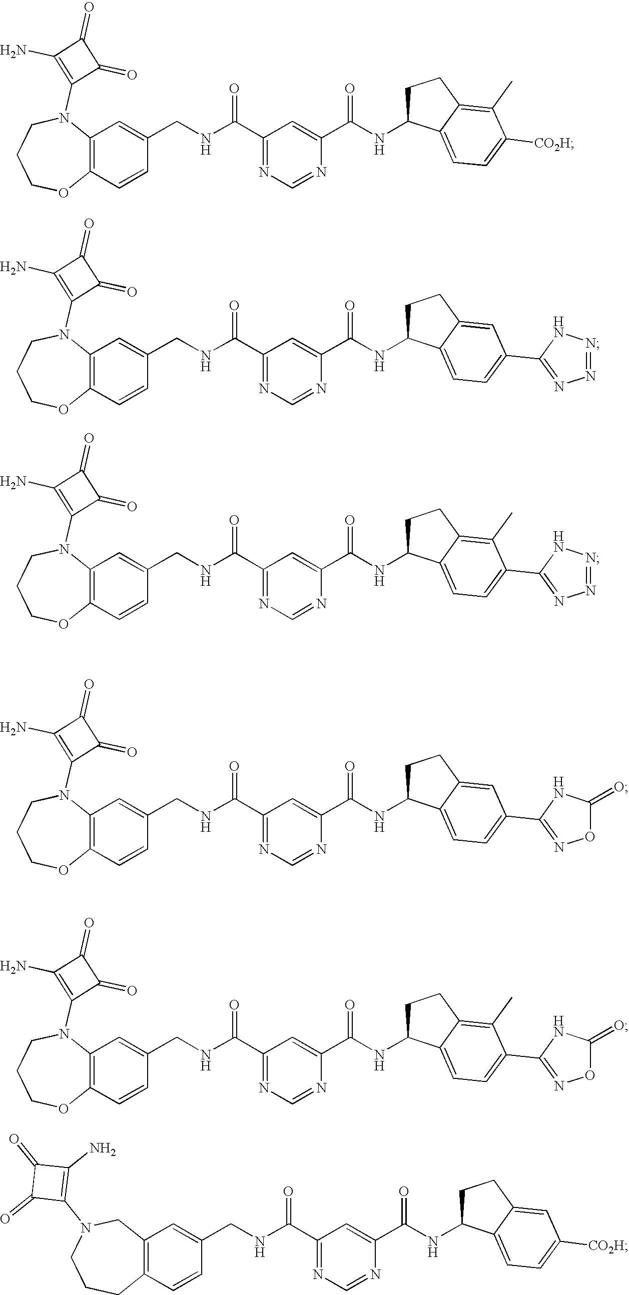

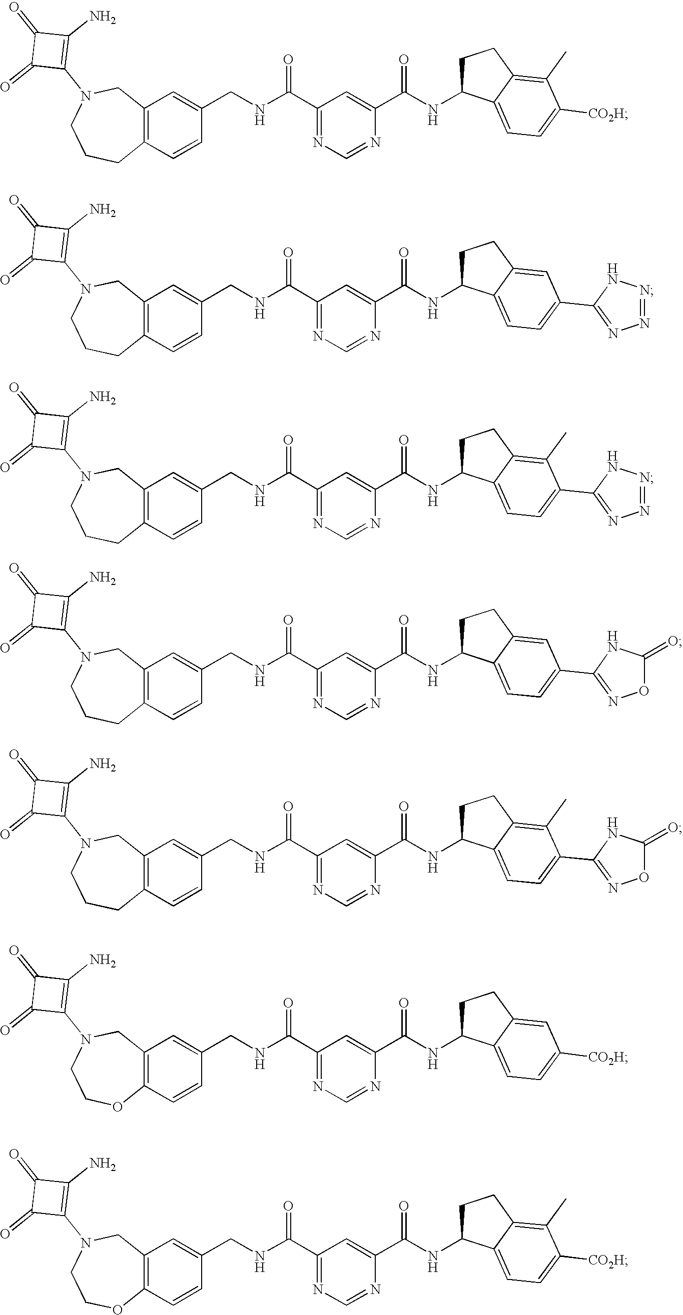

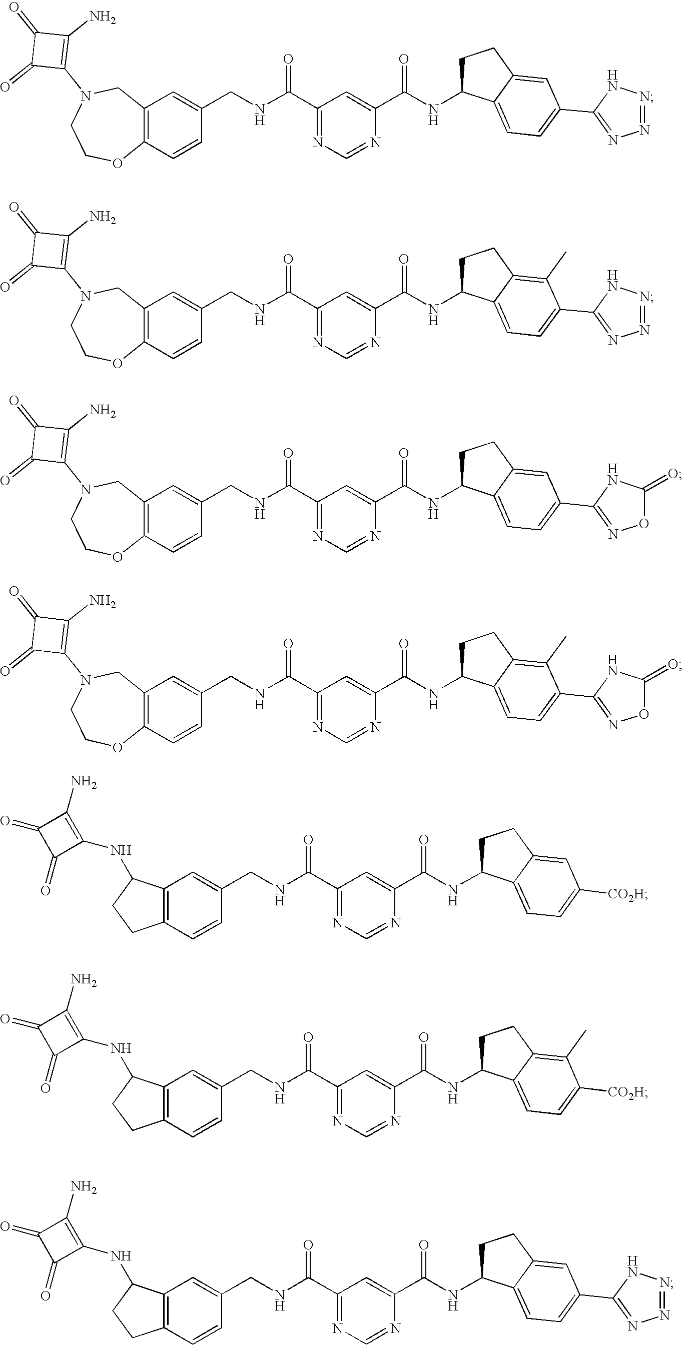

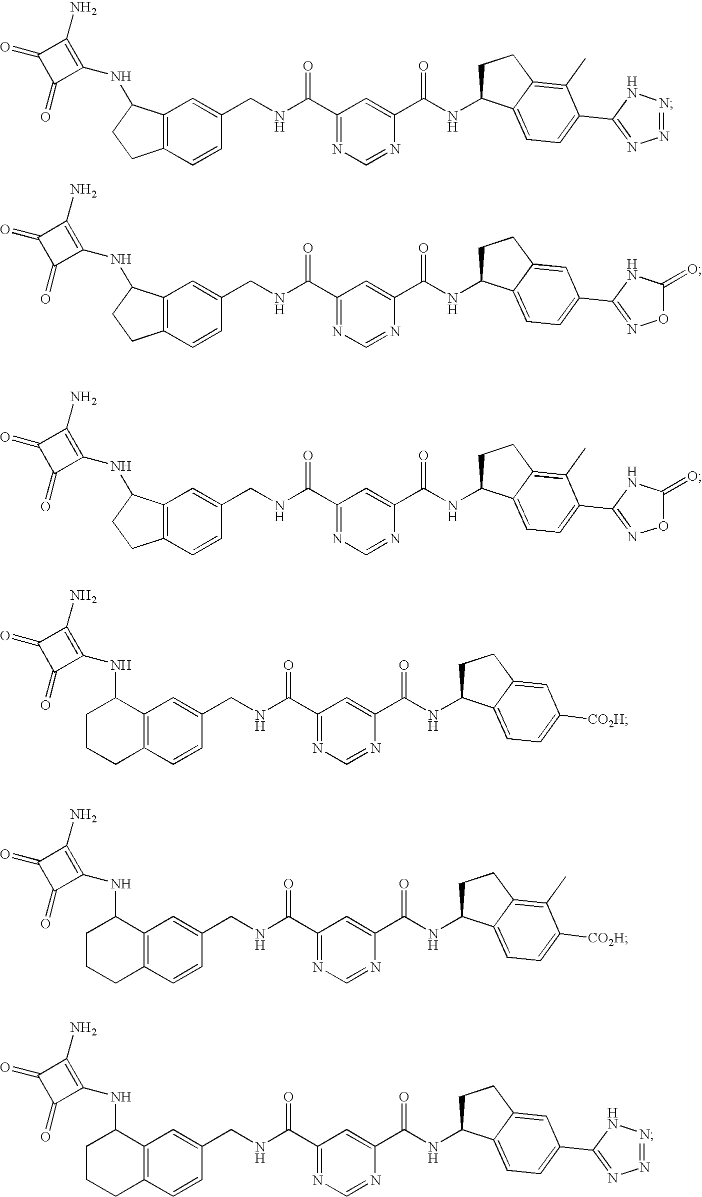

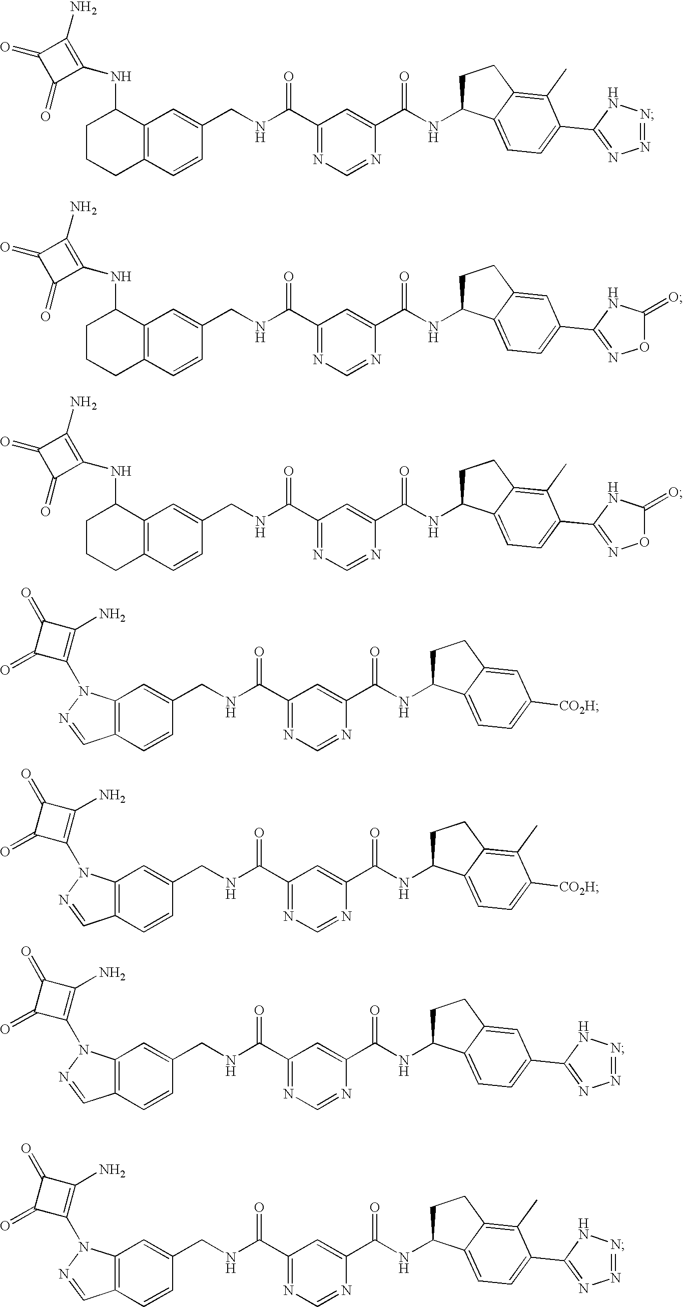

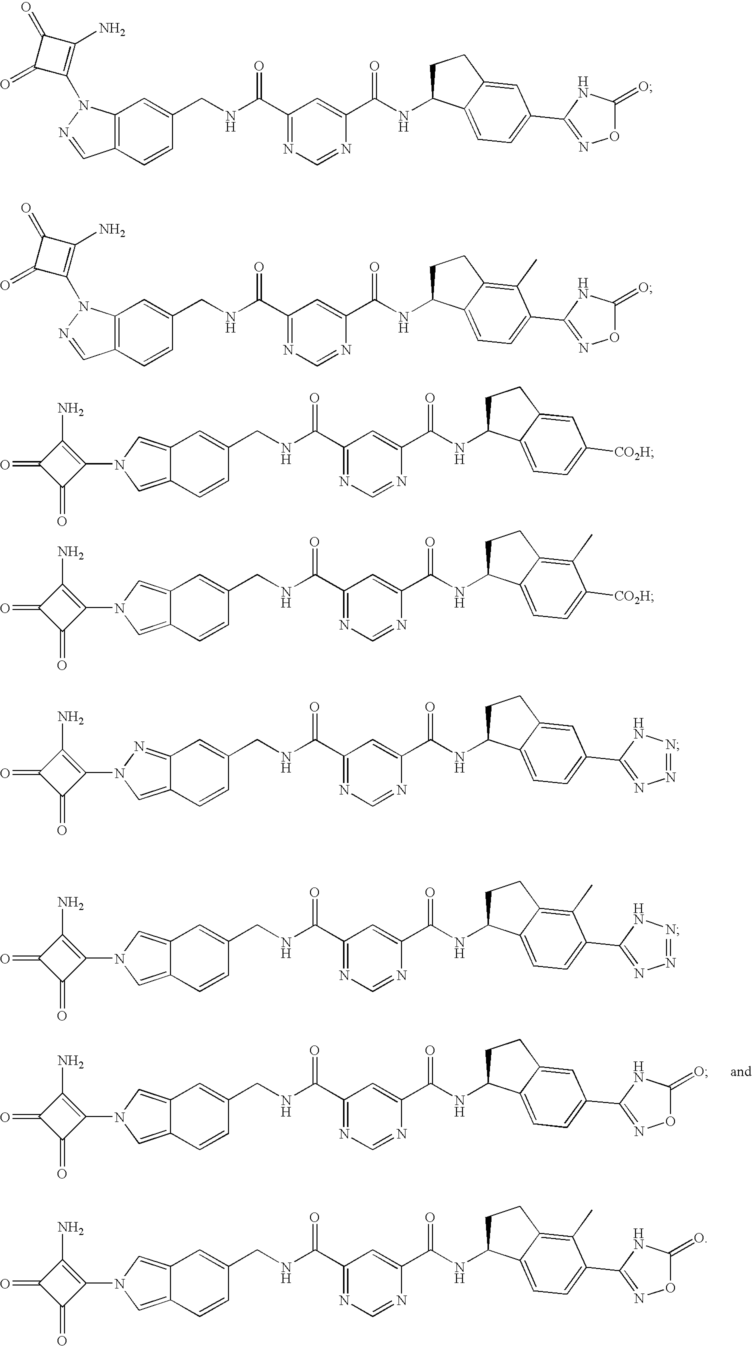

In accordance with some embodiments of the present invention, the multicyclic bis-amide MMP-13 inhibiting compounds are represented by the general Formula (VI):

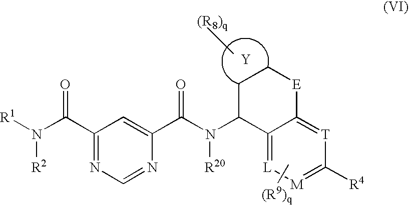

wherein:

R¹ is selected from alkyl, cycloalkyl-alkyl, arylalkyl, heteroarylalkyl and CHR²⁵R²¹, wherein alkyl, cycloalkyl-alkyl, arylalkyl and heteroarylalkyl are optionally substituted one or more times;

R is hydrogen;

R⁴ is selected from R¹⁰, hydrogen, alkyl, aryl, heteroaryl, halo, CF₃, COR¹⁰, OR¹⁰, NR¹⁰R¹¹, NO₂, CN, SO₂OR¹⁰, CO₂R¹⁰, C(O)NR¹⁰R¹¹, SO₂NR¹⁰R¹¹, SO₂R¹⁰, OC(O)R¹⁰, OC(O)NR¹⁰R¹¹, NR¹⁰C(O)R¹¹, NR¹⁰CO₂R¹¹, (C₀-C₆)-alkyl-C(═NR^{a})NHR^{b}, (C₀-C6)-alkyl-NHC(═NR^{a})NHR^{b}, (C₀-C₆)-alkyl-C(O)OR¹⁰, (C₀-C₆)-alkyl-C(O)NR¹⁰R¹¹, (C₀-C₆)-alkyl-C(O)—NH—CN, O—(C₀-C₆)-alkyl-C(O)NR¹⁰R¹¹, S(O)ₓ—(C₀-C₆)-alkyl-C(O)OR¹⁰, S(O),—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰-SO₂NR¹⁰R¹¹, wherein each R⁴ group is optionally substituted by one or more R¹⁴ groups;

R⁵ is selected from hydrogen, alkyl, C(O)NR¹⁰R¹¹, aryl, arylalkyl, SO₂NR¹⁰R¹¹, C(O)OR¹⁰ and CN, wherein alkyl, aryl and arylalkyl are optionally substituted one or more times;

R⁸ is selected from hydrogen, alkyl, OR¹⁰, NR¹⁰R¹¹, CN, arylalkyl, cycloalkyl-alkyl, heteroarylalkyl, heterocyclylalkyl and halo, wherein alkyl, arylalkyl, cycloalkyl-alkyl, heteroarylalkyl and heterocyclylalkyl are optionally substituted one or more times;

R⁹ is selected from hydrogen, alkyl, CH(CH₃)CO₂H, halo, (C₀-C₆)-alkyl-C(O)OR¹⁰, (C₀-C₆)-alkyl-C(O)NR¹⁰R¹¹, (C₀-C₆)-alkyl-C(O)NH—CN, O—(C₀-C₆)-alkyl-C(O)NR¹⁰R¹¹, S(O)_{y}-alkyl-C(O)OR¹⁰, S(O)_{z}-alkyl-C(O)NR¹⁰R¹¹, (C₀-C₆)-alkyl-C(O)NR¹⁰—(C₀-C₆)-alkyl-NR¹⁰R¹¹, C(O)NR¹⁰—(C₀-C₆)-alkyl-heteroaryl, C(O)NR¹⁰—(C₀-C₆)-alkyl-aryl, CH₂NR¹⁰R¹¹, (CH₂)_{y}NR¹⁰C(O)-alkyl, (CH₂)_{w}NR¹⁰C(O)—(C₀-C₆)-alkyl-aryl, (CH₂)_{w}NR¹⁰C(O)—(C₀-C₆)-alkyl-heteroaryl, (CH₂)_{w}NR¹⁰C(O)O-alkyl, (CH₂)_{w}NR¹⁰C(O)O—(C₀-C₆)-alkyl-aryl, (CH₂)_{w}NR¹⁰C(O)O—(C₀-C₆)-alkyl-heteroaryl, (CH₂)_{w}NR¹⁰C(O)ONR¹⁰R¹¹, (CH₂)_{w}NR¹⁰S(O)₂—(C₀-C₆)-alkyl-aryl, (CH₂)_{w}NR¹⁰S(O)₂—(C₀-C₆)-alkyl-heteroaryl, (CH₂)_{w}NR¹⁰S(O)₂—NR¹⁰-alkyl, (CH₂)_{w}NR¹⁰S(O)₂NR¹⁰—(C₀-C₆)-alkyl-aryl, (CH₂)_{w}NR¹⁰S(O)₂NR¹⁰—(C₀-C₆)-alkyl-heteroaryl, (CH₂)_{w}NR¹⁰C(O)NR¹⁰—SO₂—R³⁰ S(O)₂NR¹⁰—(C₀-C₆)-alkyl-aryl, S(O)₂NR¹⁰—(C₀-C₆-alkyl-heteroaryl, S(O)₂NR¹⁰-alkyl, S(O)₂—(C₀-C₆)-alkyl-aryl, S(O)₂—(C₀-C₆)-alkyl-heteroaryl, O-heteroaryl and heteroaryl, wherein each of said R⁹ groups is optionally substituted one or more times;

R¹⁰ and R¹¹ are independently selected from hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or R¹⁰ and R¹¹ when taken together with the nitrogen to which they are attached complete a 3- to 8-membered ring containing carbon atoms and optionally containing a heteroatom selected from O, S, or NR⁵⁰ and which is optionally substituted one or more times;

R¹⁴ is selected from hydrogen, alkyl, arylalkyl, cycloalkyl-alkyl, heteroarylalkyl, heterocyclylalkyl and halo, wherein alkyl, arylalkyl, cycloalkyl-alkyl, heteroarylalkyl and heterocyclylalkyl are optionally substituted one or more times;

R²⁰ is selected from hydrogen and alkyl, wherein alkyl is optionally substituted one or more times;

R²⁵ is selected from hydrogen, alkyl, cycloalkyl, C(O)NR¹⁰R¹¹ and haloalkyl, wherein alkyl, cycloalkyl, and haloalkyl are optionally substituted one or more times;

R³⁰ is selected from alkyl and (C₀-C₆)-alkyl-aryl;

R⁵⁰ is selected from hydrogen, alkyl, aryl, heteroaryl, C(O)R⁸⁰, C(O)NR⁸⁰R⁸¹, SO₂R⁸⁰ and SO₂NR⁸⁰R⁸¹, wherein alkyl, aryl and heteroaryl are optionally substituted one or more times;

R⁸⁰ and R⁸¹ are independently selected from hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or R⁸⁰ and R⁸¹ when taken together with the nitrogen to which they are attached complete a 3- to 8-membered ring containing carbon atoms and optionally a heteroatom selected from O, S(O)ₓ, —NH, and —N(alkyl) and which is optionally substituted one or more times;

R^{a} and R^{b} are independently selected from hydrogen, CN, alkyl, haloalkyl, S(O)ₓNR¹⁰R¹¹, S(O)ₓR¹⁰ and C(O)NR¹⁰R¹¹, wherein alkyl and haloalkyl are optionally substituted one or more times;

E is selected from a bond, CR¹⁰R¹¹, O, NR⁵, S, S═O, S(═O)₂, C(═O), N(R¹⁰)(C═O), (C═O)N(R¹⁰), N(R¹⁰)S(═O)₂, S(═O)₂N(R¹⁰), C═N—OR¹¹, —C(R¹⁰R¹¹)C(R¹⁰R¹¹)—, —CH₂—W— and

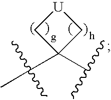

W is selected from O, NR⁵, S, S═O, S(═O)₂, N(R¹⁰)(C═O), N(R¹⁰)S(═O)₂ and S(═O)₂N(R¹⁰);

U is selected from C(R⁵R¹⁰), NR⁵, O, S, S═O and S(═O)₂;

Y is absent or selected from 3-7 membered cycloalkyl, 4-7 membered heterocyclyl, 5-6 membered heteroaryl and 6-membered aryl;

L, M and T are independently selected from C and N;

g and h are independently selected from 0-2;

q is selected from 0-4;

w is selected from 0-4;

x is selected from 0-2;

y is selected from 1 and 2; and

z is selected from 0-2.

In accordance with the definitions provided above, the compounds of Formula (VI) may include either a bicyclic or tricyclic ring system. At least one of the rings in the bicyclic or tricyclic ring system is at least partially saturated.

It is contemplated that the compounds of the present invention represented by the Formulas described above include all diastereomers and enantiomers, as well as racemic mixtures. Racemic mixtures may be separated by chiral salt resolution or by chiral column HPLC chromatography.

The present invention also is directed to pharmaceutical compositions including any of the multicyclic bis-amide MMP-13 inhibiting compounds of the present invention described above. In accordance therewith, some embodiments of the present invention provide a pharmaceutical composition which may include an effective amount of a multicyclic bis-amide MMP-13 inhibiting compound of the present invention and a pharmaceutically acceptable carrier.

The present invention also is directed to methods of inhibiting MMP-13 and methods of treating diseases or symptoms mediated by an MMP-13 enzyme. Such methods include administering a multicyclic bis-amide MMP-13 inhibiting compound of the present invention, such as a compound of Formula (I), as defined above, or an N-oxide, pharmaceutically acceptable salt or stereoisomer thereof. Examples of diseases or symptoms mediated by an MMP-13 enzyme include, but are not limited to, rheumatoid arthritis, osteoarthritis, abdominal aortic aneurysm, cancer, inflammation, atherosclerosis, multiple sclerosis, chronic obstructive pulmonary disease, ocular diseases, neurologic diseases, psychiatric diseases, thrombosis, bacterial infection, Parkinson's disease, fatigue, tremor, diabetic retinopathy, vascular diseases of the retina, aging, dementia, cardiomyopathy, renal tubular impairment, diabetes, psychosis, dyskinesia, pigmentary abnormalities, deafness, inflammatory and fibrotic syndromes, intestinal bowel syndrome, allergies, Alzheimers disease, arterial plaque formation, viral infection, stroke, atherosclerosis, cardiovascular disease, reperfusion injury, trauma, chemical exposure or oxidative damage to tissues, pain, inflammatory pain, bone pain and joint pain.

In some embodiments of the present invention, the multicyclic bis-amide MMP-13 inhibiting compounds defined above are used in the manufacture of a medicament for the treatment of a disease mediated by an MMP-13 enzyme.

In some embodiments, the multicyclic bis-amide MMP-13 inhibiting compounds defined above may be used in combination with a drug, agent or therapeutic such as, but not limited to: (a) a disease modifying antirheumatic drug; (b) a nonsteroidal anti-inflammatory drug; (c) a COX-2 selective inhibitor; (d) a COX-1 inhibitor; (e) an immunosuppressive; (f) a steroid; (g) a biological response modifier; or (h) other anti-inflammatory agents or therapeutics useful for the treatment of chemokine mediated diseases.

Examples of disease modifying antirheumatic drugs include, but are not limited to, methotrexate, azathioptrineluflunomide, penicillamine, gold salts, mycophenolate, mofetil and cyclophosphamide.

Examples of nonsteroidal anitinflammatory drugs include, but are not limited to, piroxicam, ketoprofen, naproxen, indomethacin, and ibuprofen.

Examples of COX-2 selective inhibitors include, but are not limited to, rofecoxib, celecoxib, and valdecoxib.

An example of a COX-1 inhibitor includes, but is not limited to, piroxicam.

Examples of immunosuppressives include, but are not limited to, methotrexate, cyclosporin, leflunimide, tacrolimus, rapamycin and sulfasalazine.

Examples of steroids include, but are not limited to, p-methasone, prednisone, cortisone, prednisolone and dexamethasone.

Examples of biological response modifiers include, but are not limited to, anti- TNF antibodies, TNF-α antagonists, IL-1 antagonists, anti- CD40, anti-CD28, IL-10 and anti-adhesion molecules.

l Examples of anti-inflammatory agents or therapeutics include, but are not limited to, p38 kinase inhibitors, PDE4 inhibitors, TACE inhibitors, chemokine receptor antagonists, thalidomide, leukotriene inhibitors and other small molecule inhibitors of pro-inflammatory cytokine production.

In accordance with another embodiment of the present invention, a pharmaceutical composition may include an effective amount of a compound of the present invention, a pharmaceutically acceptable carrier and a drug, agent or therapeutic selected from: (a) a disease modifying antirheumatic drug; (b) a nonsteroidal anti-inflammatory drug; (c) a COX-2 selective inhibitor; (d) a COX-1 inhibitor; (e) an immunosuppressive; (f) a steroid; (g) a biological response modifier; or (h) other anti-inflammatory agents or therapeutics useful for the treatment of chemokine mediated diseases.

In some embodiments of the present invention, the compounds of Formula I are synthesized by the general method shown in Scheme 1.

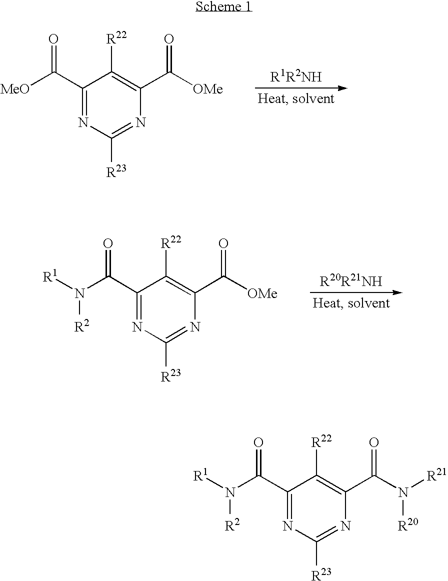

Dimethyl pyrimidine-4,6-dicarboxylate (R²²═R²³═H) is treated with a slight molar excess of R¹R²NH in a suitable solvent and heated to afford the desired adduct after purification. This compound is further treated with a slight molar excess of R²⁰R²¹NH in a suitable solvent and heated to give the final desired adduct after purification. Alternatively, the final adduct can be obtained by one skilled in the art through comparable coupling reactions.

In some embodiments the compounds of Formula I are synthesized by the general method shown in Scheme 2.

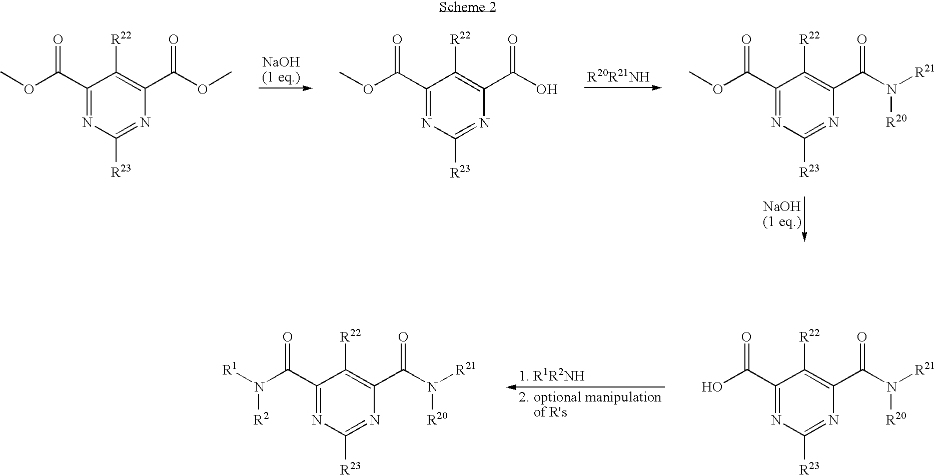

A dimethyl pyrimidine-4,6-dicarboxylate derivative is treated with one equivalent sodium hydroxide to give the monomethyl pyrimidine-4,6-dicarboxylate derivative. After an activated acid coupling (e.g. HOBt/EDCI, HOAt/HATU, PyBroP or ethyl chloroformate) of R²⁰R²¹NH in a suitable solvent afford the desired adduct after purification. This compound is further treated with one equivalent sodium hydroxide and then coupled via an activated acid (e.g. HOBt/EDCI, HOAt/HATO, PyBroP or ethyl chloroformate) with R¹R²NH to give the pyrimidine-4,6-bis-amide. If necessary, the R group can be further manipulated (e.g. saponification of a COOMe group in R).

The MMP-13 inhibiting activity of the multicyclic bis-amide MMP-13 inhibiting compounds of the present invention may be measured using any suitable assay known in the art. A standard in vitro assay for MMP-13 inhibiting activity is described in Example 3000.

The multicyclic bis-amide MMP-13 inhibiting compounds of the invention have an MMP-13 inhibition activity (IC₅₀ MMP-13) ranging from about 1 nM to about 20 μM, and typically, from about 8 nM to about 2 μM. Multicyclic bis-amide MMP-13 inhibiting compounds of the invention desirably have an MMP inhibition activity ranging from about 1 nM to about 20 nM. Table 1 lists typical examples of multicyclic bis-amide MMP-13 inhibiting compounds of the invention that have an MMP-13 activity lower than about 1 μM, particularly about 1 nM to 300 nM, and more specifically about 1 nM to 260 nM.

TABLE 1
Summary of MMP-13 Activity for Compounds of Formula I

| Compound | | |
|---|---|---|
| No. | Structure | IC₅₀ |
| Ex. 1 | 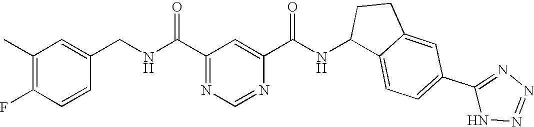 | >5 nM |
| Ex. 2301 | 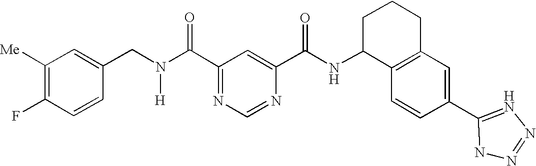 | >5 nM |
| Ex. 2303 | 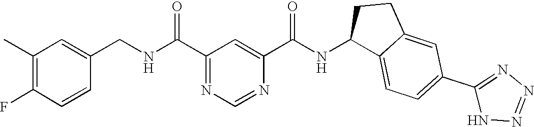 | >5 nM |
| Ex. 2308 | 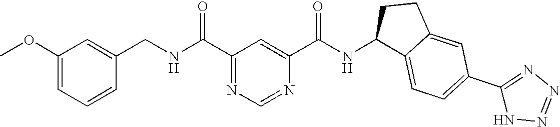 | >5 nM |
| Ex. 2328 | 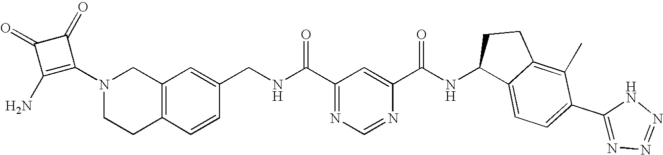 | <5 nM |
| Ex. 2407 | 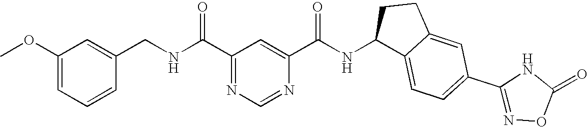 | <5 nM |
| Ex. 2522 | 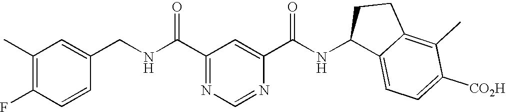 | <5 nM |
| Ex. 2539 | 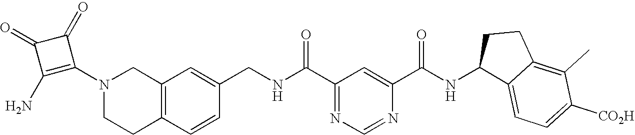 | <5 nM |
| Ex. 2562 | 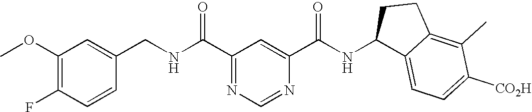 | <5 nM |
| Ex. 2702 | 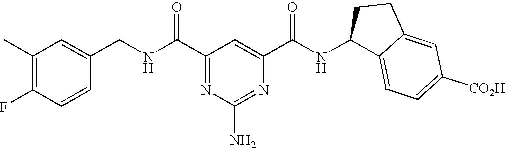 | <5 nM |

The synthesis of multicyclic bis-amide MMP-13 inhibiting compounds of the invention and their biological activity assay are described in the following examples which are not intended to be limiting in any way.

### EXAMPLES AND METHODS

All reagents and solvents were obtained from commercial sources and used without further purification. Proton (¹H) spectra were recorded on a 400 MHz NMR spectrometer in deuterated solvents. Flash chromatography was performed using Merck silica gel, grade 60, 70-230 mesh using suitable organic solvents as indicated in specific examples. Thin layer chromatography (TLC) was carried out on silica gel plates with UV detection.

Preparative Examples 1, 3, 5, 8, 9a, 10-152, 2001-2067 and 2100-2125 are directed to intermediate compounds useful in preparing the compounds of the present invention.

### Preparative Example 1

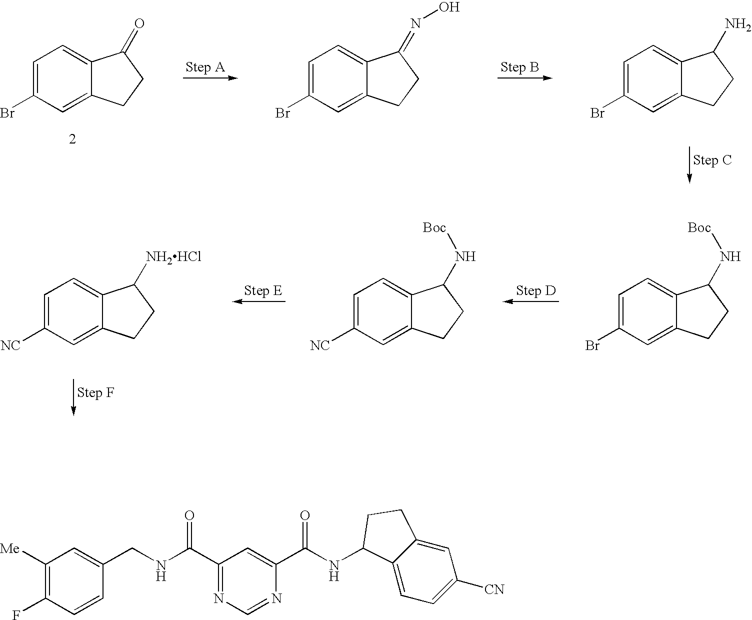 Step A

A mixture of 5-bromo-1-indanone (1.76 g), NH₂OH.HCl (636 mg) and NaOAc (751 mg) in MeOH (40 mL) was allowed to stir for 16 h at 22° C. Water (100 mL) was added and the resulting precipitate was filtered and washed three times with water (20 mL) to afford a colourless solid (1.88 g; >99%). [MH]⁺=226.

Step B

To a mixture of 5-bromo-indan-1-one oxime (1.88 g) in Et₂O (20 mL) at −78° C. under an atmosphere of Ar was slowly added a 1 M solution of lithium aluminum hydride in Et₂O (42.4 mL). The mixture was heated to reflux (40° C.) and allowed to stir for 5 h. The mixture was cooled to 0° C. and water (1.6 mL), 15% aqueous NaOH (1.6 mL), and water (4.8 mL) were carefully and sequentially added. The resulting mixture was filtered through Celite and the filtrate was concentrated to give a clear oil (1.65 g; 94%). [MH]⁺=212.

Step C

A solution of 5-bromo-indan-1-ylamine (300 mg), di-tert-butyl-dicarbonate (370 mg), and triethyl amine (237 μL) in THF (10 mL) was allowed to stir at 22° C. for 16 h. The solution was concentrated and the resulting residue was purified through a short column of silica gel (4:1 hexanes: ethyl acetate, R_{f}=0.3) to give a clear oil (460 mg; >99%).

Step D

A mixture of (5-bromo-indan-1-yl)-carbamic acid tert-butyl ester (460 mg), Pd(PPh₃)₄ (89 mg), Zn(CN)₂ (200 mg), and DMF (5 mL) under an atmosphere of Ar in a sealed vial was allowed to stir at 110° C. for 18 h. The mixture was allowed to cool to 22° C., Et₂O (20 mL) and water (20 mL) were added. The aqueous layer was washed four times with Et₂O (10 mL). The combined organic layers were washed three times with water (10 mL), once with brine (10 mL), dried over MgSO₄, filtered and concentrated. The resulting residue was purified by silica gel chromatography (4:1 hexanes: ethyl acetate, R_{f}=0.2) to afford a clear oil (170 mg; 47%). [MH]⁺=259.

Step E

To (5-cyano-indan-1-yl)-carbamic acid tert-butyl ester (170 mg) was added a solution of 4M HCl in dioxane (2 mL) and the resulting solution was allowed to stir at 22° C. for 3 h at which time a precipitate had formed. The mixture was concentrated to give a colourless powder (128 mg; >99%). [M-Cl⁻]⁺=159.

Step F

To a mixture of 5-cyano-indan-1-yl-ammonium chloride (50.6 mg), 6-(4-Fluoro-3-methyl-benzylcarbamoyl)-pyrimidine-4-carboxylic acid (62.7 mg) prepared in Preparative Example 2120, Bromotripyrrolidinophosphonium hexafluorophosphate (124 mg) in THF (2 mL) was added triethyl amine (67 μL). The mixture was allowed to stir at 22° C. for 18 h. EtOAc (10 mL) and 1N aqueous HCl (10 mL) were added. The aqueous layer was washed two times with EtOAc (10 mL). The combined organic layers were washed with a saturated aqueous solution of NaHCO₃ (10 mL), brine (10 mL), dried over MgSO₄, filtered and concentrated. The resulting residue was purified by silica gel chromatography (1:1 hexanes: ethyl acetate, R_{f}=0.3) to afford an off-white solid (75.8 mg; 81%).

### Preparative Example 3

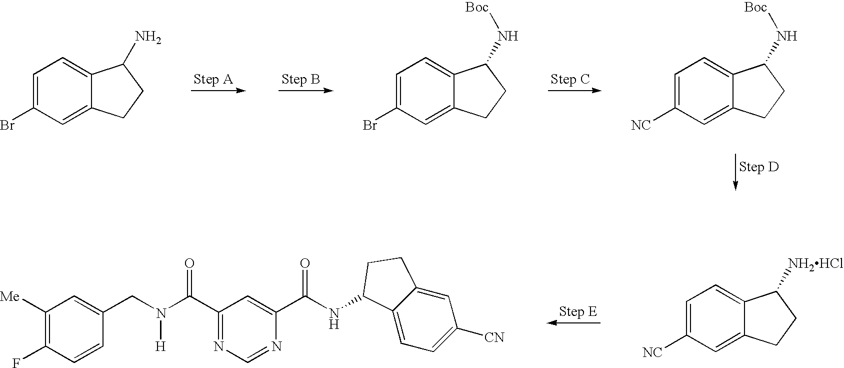 Step A

A solution of 5-bromo-indan-1-ylamine (300 mg), di-tert-butyl-dicarbonate (370 mg), and triethyl amine (237 μL) in THF (10 mL) was allowed to stir at 22° C. for 16 h. The solution was concentrated and the resulting residue was purified through a short column of silica gel (4:1 hexanes: ethyl acetate, R_{f}=0.3) to give a clear oil (460 mg; >99%).

Step B

To a boiling solution of racemic 5-bromo-indan-1-ylamine (1.13 g) in MeOH (2.3 mL) was added a hot solution of N-acetyl-D-leucine (924 mg) in MeOH (3 mL). The solution was allowed to cool to 22° C., which afforded a white precipitate. The solid was separated from the supernatant and washed with MeOH (2 mL). The solid was recrystalized two times from MeOH. To the resulting solid were added a 10% aqueous solution of NaOH (20 mL) and Et₂O (20 mL). Once the solid was dissolved (5 min) the organic layer was removed and the aqueous layer was washed two times with Et₂O. The combined organic layers were dried over MgSO₄, filtered and concentrated to give a clear oil (99 mg; 18%). [MH]⁺=212.

Step C

A mixture of (5-bromo-indan-1-yl)-carbamic acid tert-butyl ester (460 mg), Pd(PPh₃)₄ (89 mg), Zn(CN)₂ (200 mg), and DMF (5 mL) under an atmosphere of Ar in a sealed vial was allowed to stir at 110° C. for 18 h. The mixture was allowed to cool to 22° C., Et₂O (20 mL) and water (20 mL) were added. The aqueous layer was washed four times with Et₂O (10 mL). The combined organic layers were washed three times with water (10 mL), once with brine (10 mL), dried over MgSO₄, filtered and concentrated. The resulting residue was purified by silica gel chromatography (4:1 hexanes: ethyl acetate, R_{f}=0.2) to afford a clear oil (170 mg; 47%). [MH]⁺=259.

Step D

To (5-cyano-indan-1-yl)-carbamic acid tert-butyl ester (170 mg) was added a solution of 4M HCl in dioxane (2 mL) and the resulting solution was allowed to stir at 22° C. for 3 h at which time a precipitate had formed. The mixture was concentrated to give a colourless powder (128 mg; >99%). [M-Cl⁻]⁺=159.

Step E

To a mixture of 5-cyano-indan-1-yl-ammonium chloride (50.6 mg), 6-(4-Fluoro-3-methyl-benzylcarbamoyl)-pyrimidine-4-carboxylic acid (62.7 mg) prepared in Preparative Example 2120, Bromotripyrrolidinophosphonium hexafluorophosphate (124 mg) in THF (2 mL) was added triethyl amine (67 μL). The mixture was allowed to stir at 22° C. for 18 h. EtOAc (10 mL) and 1N aqueous HCl (10 mL) were added. The aqueous layer was washed two times with EtOAc (10 mL). The combined organic layers were washed with a saturated aqueous solution of NaHCO₃ (10 mL), brine (10 mL), dried over MgSO₄, filtered and concentrated. The resulting residue was purified by silica gel chromatography (1:1 hexanes: ethyl acetate, R_{f}=0.3) to afford an off-white solid (75.8 mg; 81%).

### Preparative Example 5

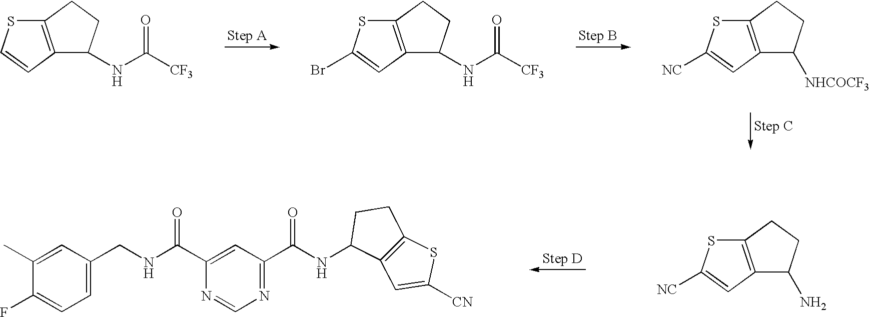 Step A

Comercially available 2,2,2-trifluoro-N-(5,6-dihydro-4H-cyclopenta[b]thiophen-4-yl)acetamide (95 mg) and AlCl₃ (5 mg) were dissolved in 2 mL of AcOH under aluminum foil. Bromine (23 μL) was added to the solution and the mixture was stirred at room temperature for 3 h. 10% aqueous Na₂S₂O₃ solution (7 mL) was added to the solution and the mixture was stirred for 10 min. EtOAc was added to the mixture and the organic layer was washed with brine, dried over MgSO₄, and concentrated in vaccuo. The residue was chromatographed on silica gel to afford 87 mg of brown solid (95%). ¹HNMR (CDCl₃) δ=2.20-2.38 (m, 1 H), 2.83-3.15 (m, 3 H), 5.37 (m, 1 H), 6.50 (s, 1 H), 6.89 (d, 1 H), 7.28 (d, 1 H). [MH]⁺=314/316.

Step B

N-(2-Bromo-5,6-dihydro-4H-cyclopenta[b]thiophen-4-yl)-2,2,2-trifluoroacetamide (87 mg), Pd₂(dba)₃ (12.7 mg) and dppf (30.8 mg) were added to anhydrous DMF (6.5 mL). The mixture was heated to 80° C. Zn(CN)₂ (39 mg) was added in portions. The mixture was stirred for 24 h. The solvent was evaporated in vaccuo. The residue was chromatographed on silica gel to afford 48 mg of white solid (66%). ¹HNMR (CDCl₃) δ=2.35-2.40 (m, 1 H), 2.95-3.25 (m, 3 H), 5.47 (m, 1 H), 6.75 (s, 1 H), 7.45 (s, 1 H). [M-H⁺]⁻259.

Step C

N-(2-Cyano-5,6-dihydro-4H-cyclopenta[b]thiophen-4-yl)-2,2,2-trifluoroacetamide (47 mg) and K₂CO₃ (142 mg) were added to MeOH (5 mL) and H₂O (3 mL). The mixture was stirred at room temperature for 16 h, diluted with H₂O and extracted with CH₂Cl₂. The organic layer was washed with brine, dried over MgSO₄, and concentrated in vaccuo. The residue was chromatographed on silica gel to afford 30 mg of off-white solid (100%). ¹HNMR (CDCl₃) δ=1.65 (s, 2 H), 2.00-2.18 (m, 1 H), 2.75-3.15 (m, 1 H), 7.44 (s, 1 H).

Step D

6-(4-Fluoro-3-methylbenzylcarbamoyl)pyrimidine-4-carboxylic acid prepared in Preparative Example 2120 (79 mg), the corresponding cyano-amine (30 mg), EDIC (53 mg) and HOBt (37 mg) were dissolved in THF (5 mL). The mixture was stirred for 16 h, and diluted with EtOAc, washed with NaHCO₃ and brine. The organic layer was washed with brine, dried over MgSO₄, and concentrated in vaccuo. The residue was chromatographed on silica gel to afford 43.8 mg of white solid (56%). [MH]⁺=436.

### Preparative Example 8

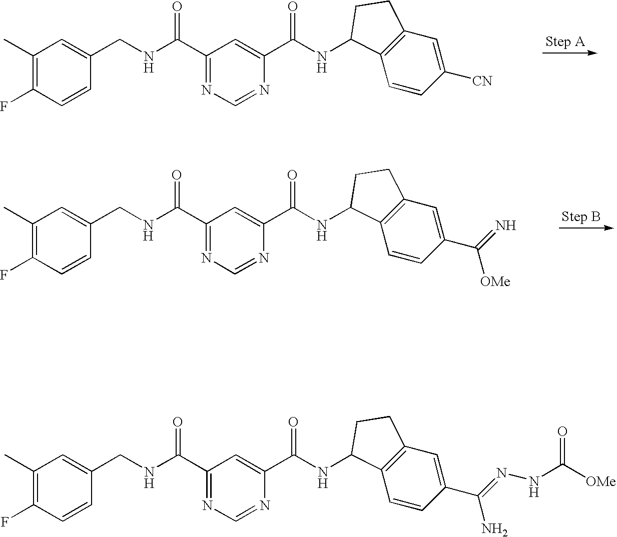 Step A

If one were to treat the starting cyano compound (306 mg) from Preparative Example 1 in dry methanol (20 mL) hydrochloride gas at 0° C., one would obtain the title compound.

Step B

If one were to treat the title compound from above dissolved in methanol (20 mL) with sodium bicarbonate (336 mg) at room temperature, one would obtain the title compound.

### Preparative Example 9a

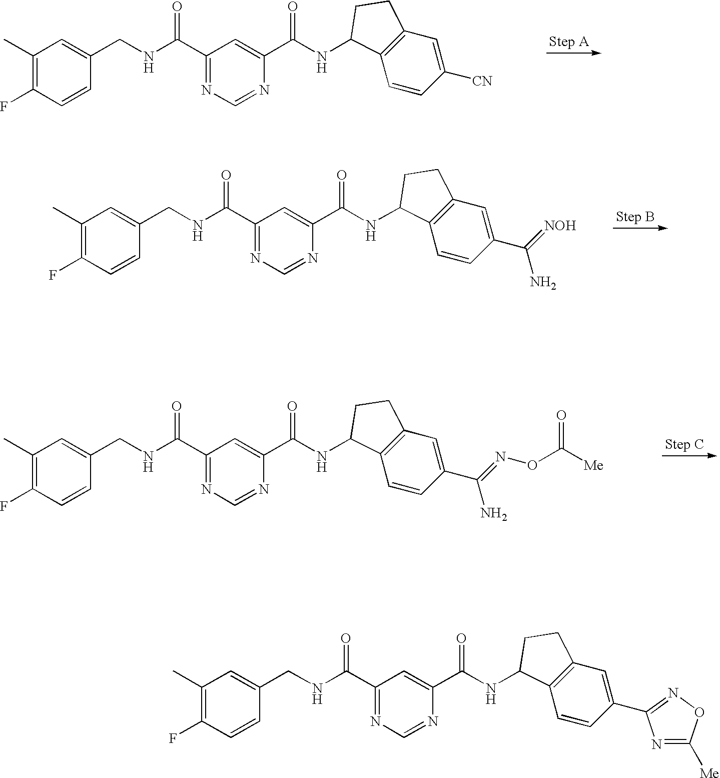 Step A

If one were to reflux the cyano compound (42 mg) from Preparative Example 1 with hydroxylamine (69 mg hydrochloride salt neutralized with grounded potassium hydroxide in ethanol) in ethanol (3 mL) overnight, one would obtain the desired amidoxime.

Step B

If one were to treat the title product from step Step A above, dissolved in tetrahydrofurane and cooled to 0° C. in ice bath with pyridine followed by acetyl chloride, one would obtain the desired compound.

Step C

If one were to reflux the title product from Step B above in chlorobenzene, one would obtain the desired oxadiazole.

### Preparative Examples 10-152

If one were to couple the amine indicated in Table 2 below with the intermediate from Preparative Example 2119, Step A (or its enantiomer) according to the procedure outlined in Example 2300, Step A and with the product from Preparative Example 2120 according to the procedure outlined in Example 1, Step F, respectively, one would obtain the Product indicated in Table 2 below.

TABLE 2

| Ex # | Amine | Coupling Agent | Product |
|---|---|---|---|
| 10 | 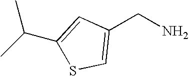 | 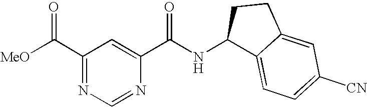 | 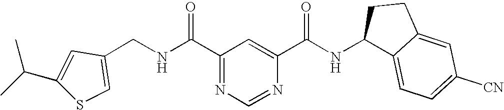 |
| 11 | 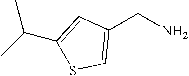 | 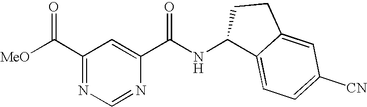 | 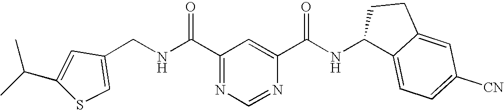 |
| 13 | 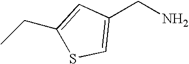 | 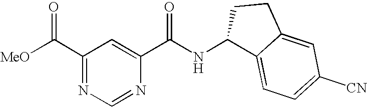 | 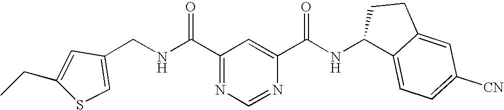 |
| 14 | 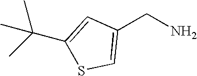 | 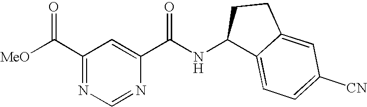 | 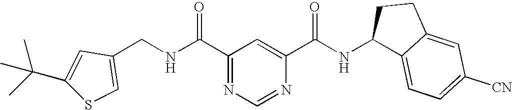 |
| 15 | 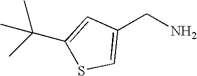 | 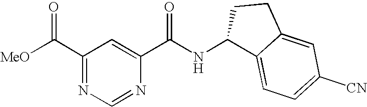 | 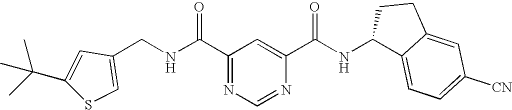 |
| 16 | 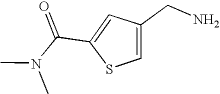 | 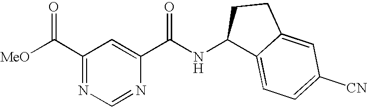 | 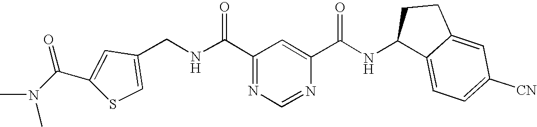 |
| 17 | 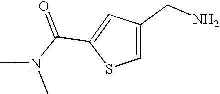 | 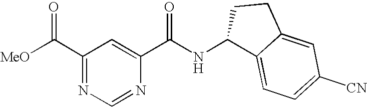 | 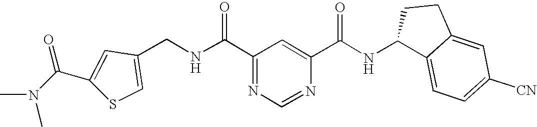 |
| 18 | 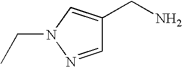 | 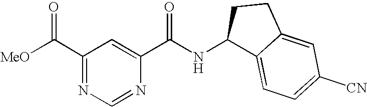 | 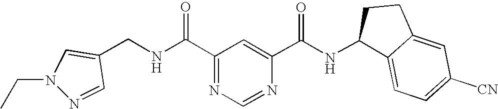 |
| 19 | 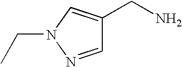 | 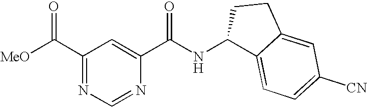 | 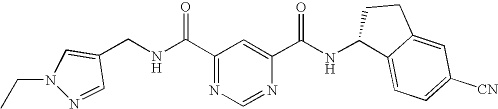 |
| 20 | 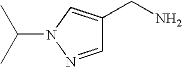 | 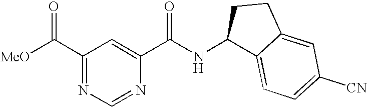 | 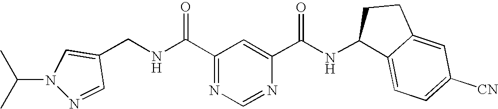 |
| 21 | 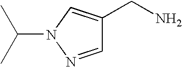 | 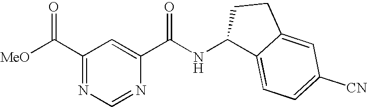 | 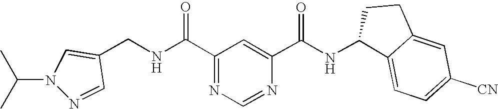 |
| 22 | 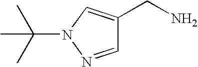 | 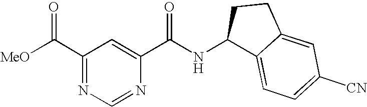 | 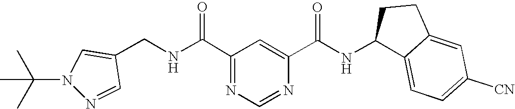 |
| 23 | 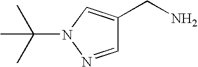 | 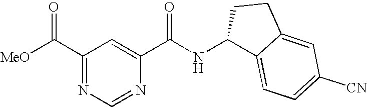 | 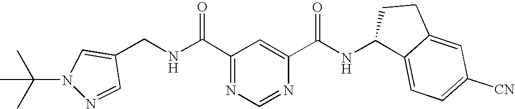 |
| 24 | 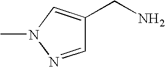 | 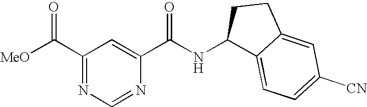 | 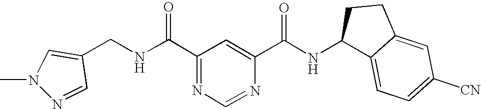 |
| 25 | 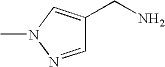 | 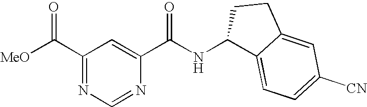 | 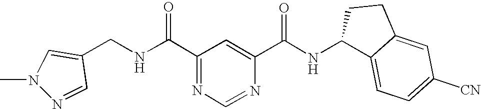 |
| 26 | 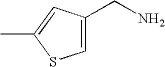 | 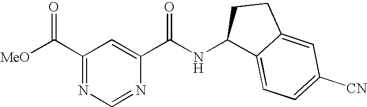 | 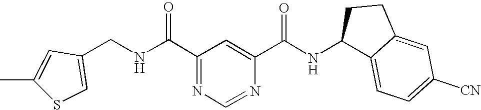 |
| 27 | 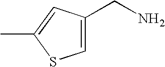 | 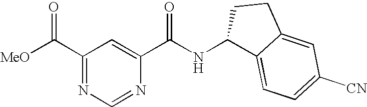 | 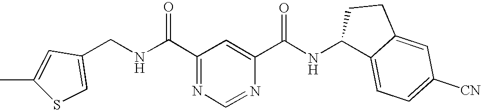 |
| 28 | 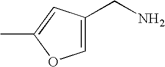 | 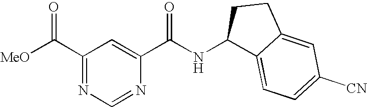 | 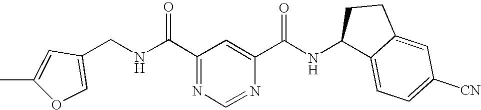 |
| 29 | 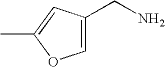 | 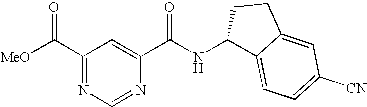 | 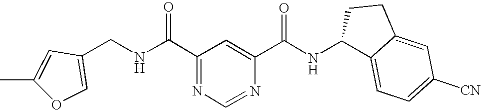 |
| 30 | 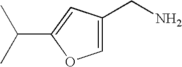 | 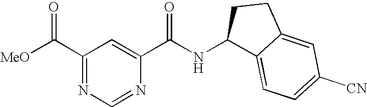 | 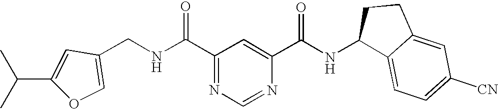 |
| 31 | 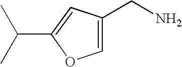 | 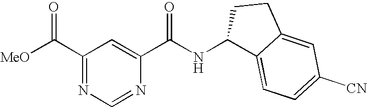 | 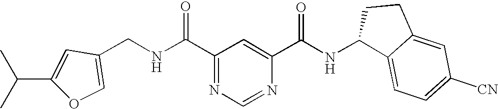 |
| 32 | 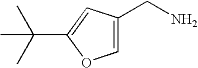 | 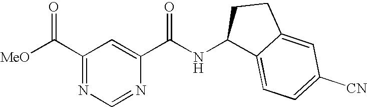 | 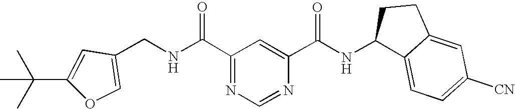 |
| 33 | 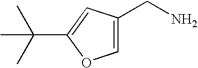 | 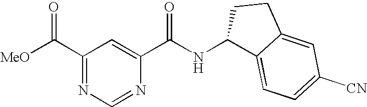 | 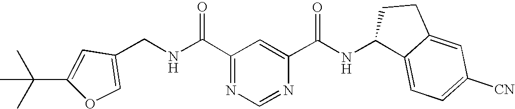 |
| 35 | 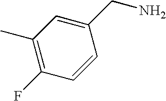 | 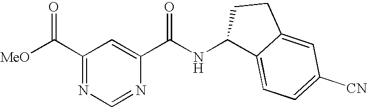 | 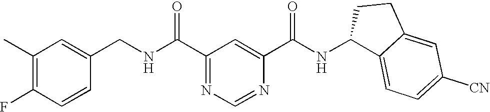 |
| 36 | 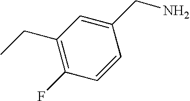 | 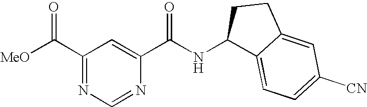 | 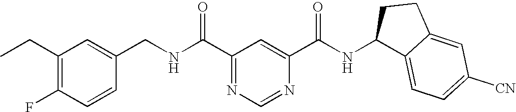 |
| 37 | 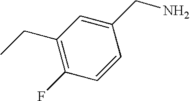 | 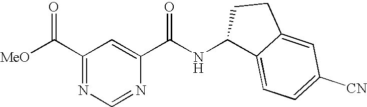 | 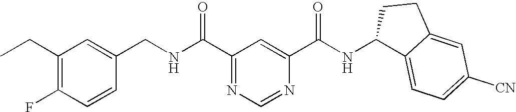 |
| 38 | 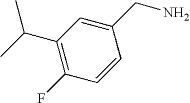 | 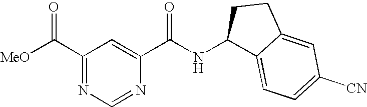 | 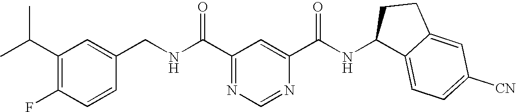 |
| 39 | 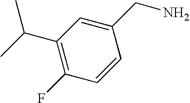 | 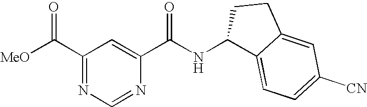 | 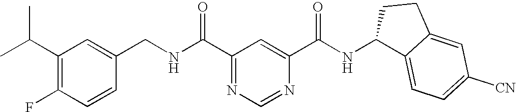 |
| 40 | 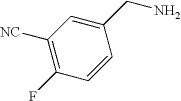 | 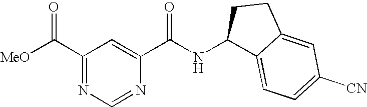 | 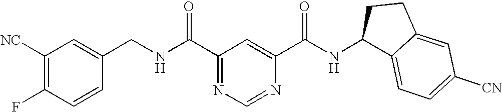 |
| 41 | 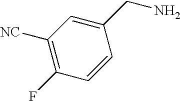 | 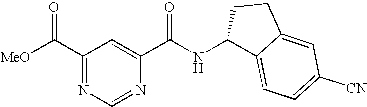 | 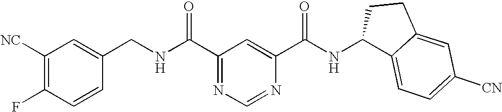 |
| 42 | 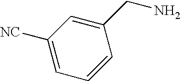 | 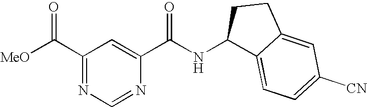 | 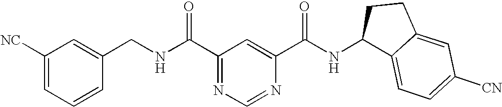 |
| 43 | 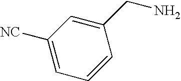 | 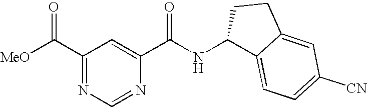 | 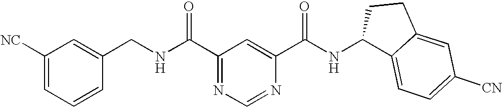 |
| 44 | 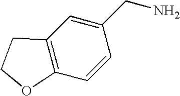 | 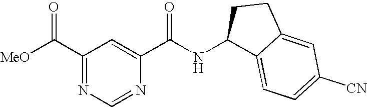 | 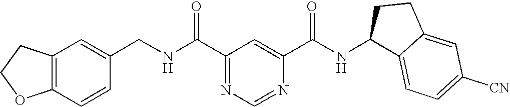 |
| 45 | 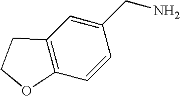 | 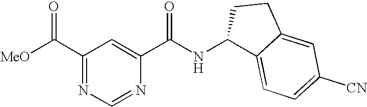 | 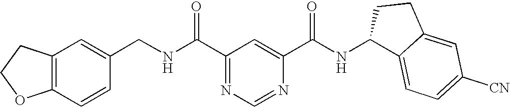 |
| 46 | 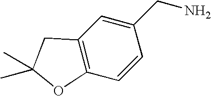 | 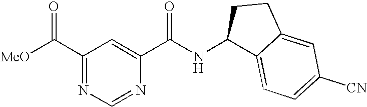 | 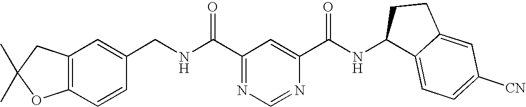 |
| 47 | 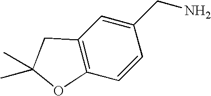 | 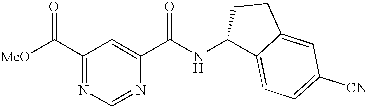 | 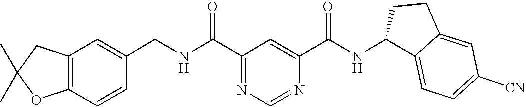 |
| 48 | 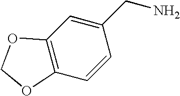 | 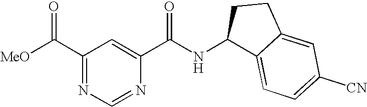 | 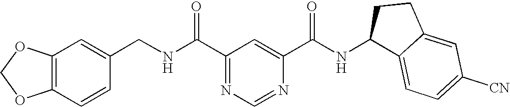 |
| 49 | 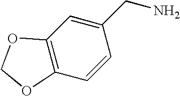 | 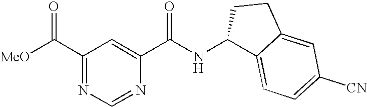 | 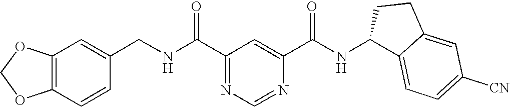 |
| 50 | 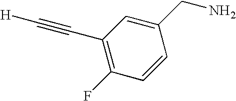 | 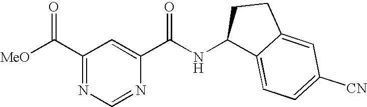 | 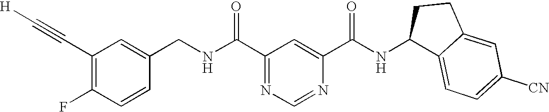 |
| 51 | 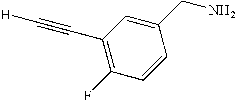 | 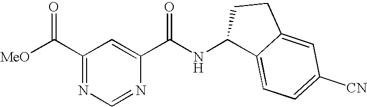 | 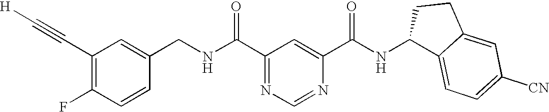 |
| 52 | 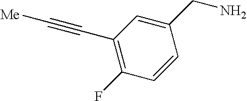 | 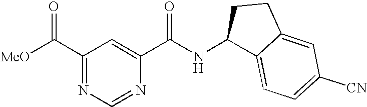 | 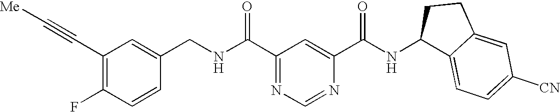 |
| 53 | 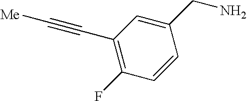 | 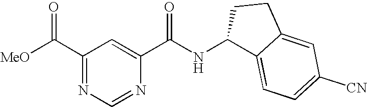 | 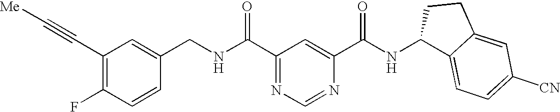 |
| 54 | 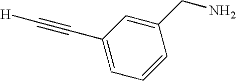 | 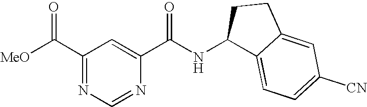 | 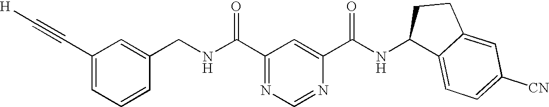 |
| 55 | 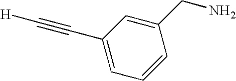 | 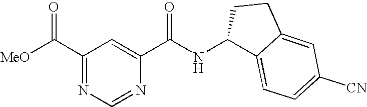 | 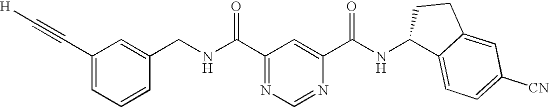 |
| 56 | 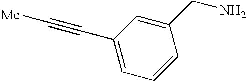 | 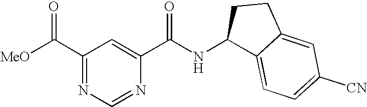 | 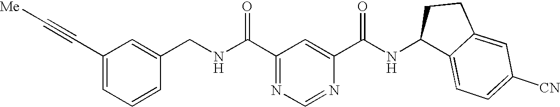 |
| 57 | 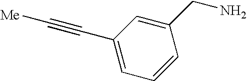 | 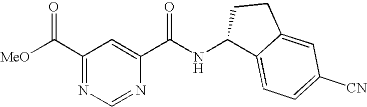 | 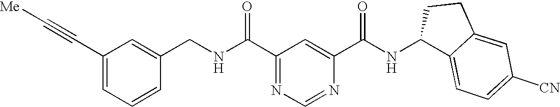 |
| 58 | 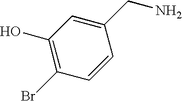 | 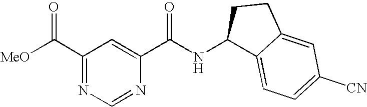 | 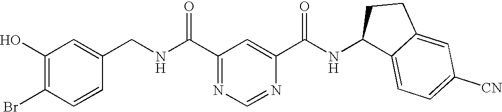 |
| 59 | 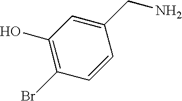 | 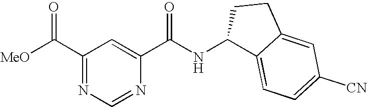 | 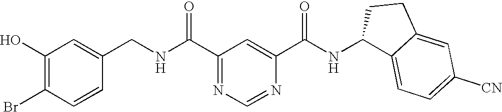 |
| 60 | 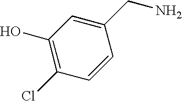 | 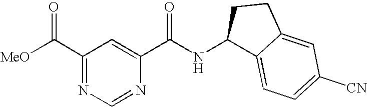 | 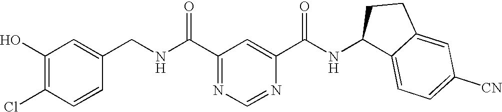 |
| 61 | 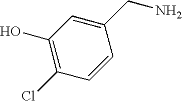 | 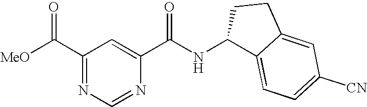 | 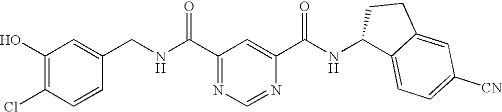 |
| 62 | 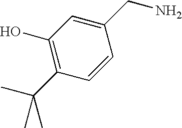 | 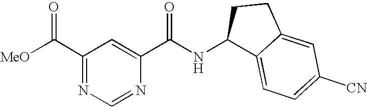 | 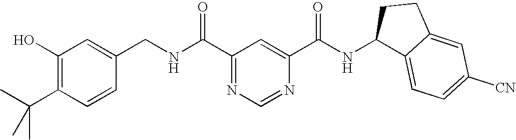 |
| 63 | 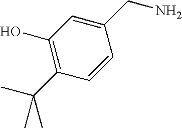 | 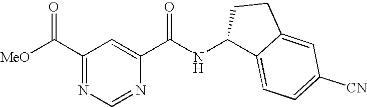 | 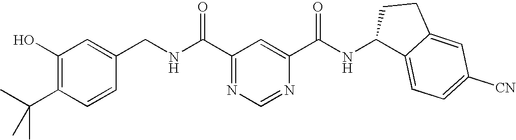 |
| 65 | 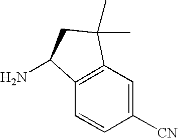 | 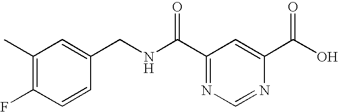 | 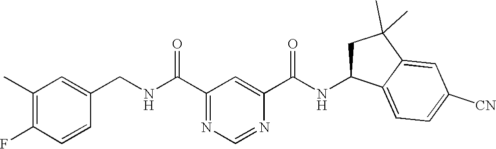 |
| 66 | 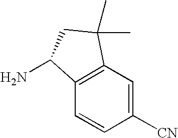 | 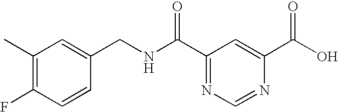 | 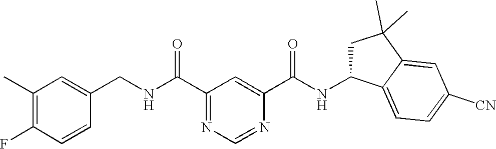 |
| 67 | 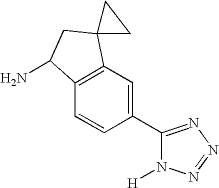 | 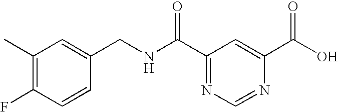 | 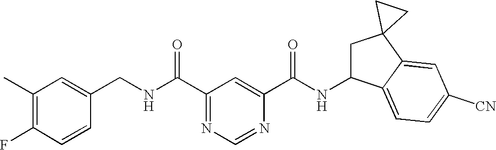 |
| 68 | 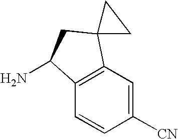 | 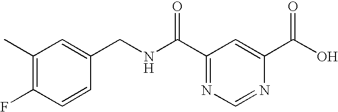 | 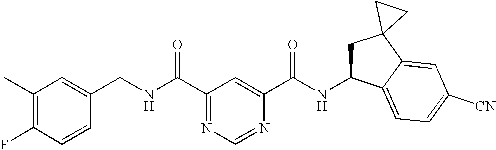 |
| 69 | 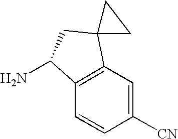 | 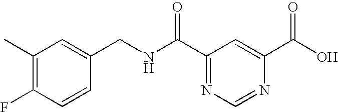 | 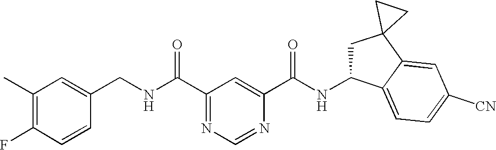 |
| 71 | 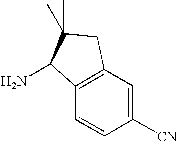 | 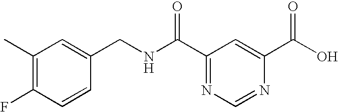 | 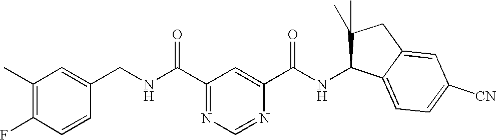 |
| 72 | 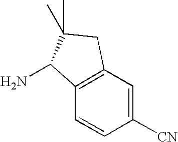 | 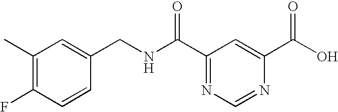 | 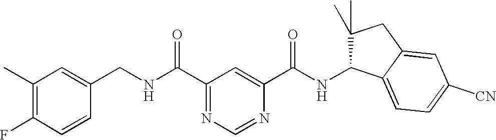 |
| 73 | 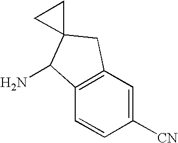 | 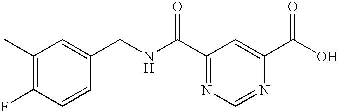 | 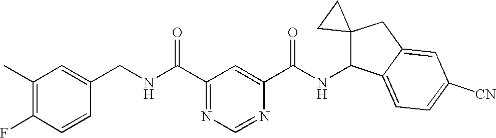 |
| 74 | 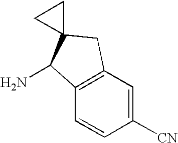 | 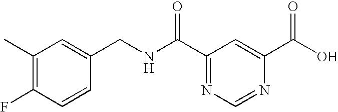 | 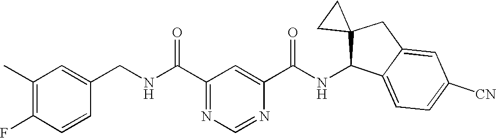 |
| 75 | 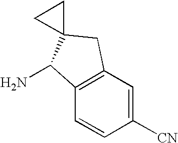 | 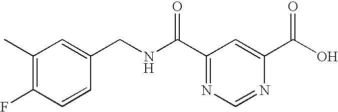 | 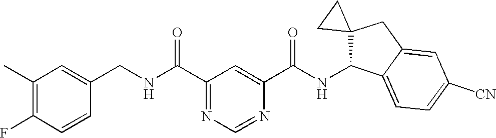 |
| 76 | 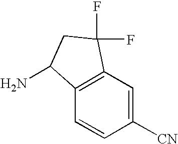 | 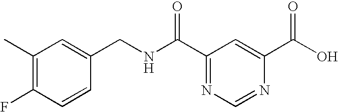 | 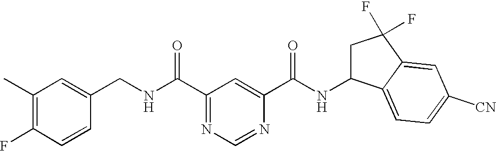 |
| 77 | 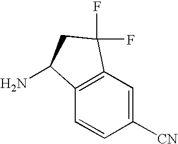 | 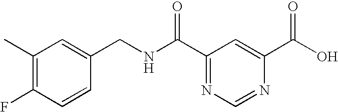 | 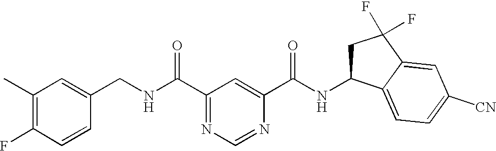 |
| 78 | 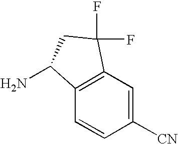 | 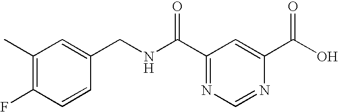 | 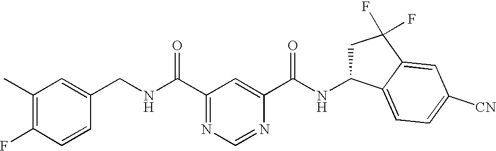 |
| 79 | 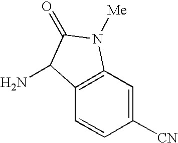 | 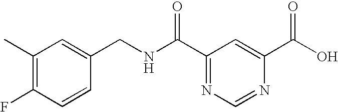 | 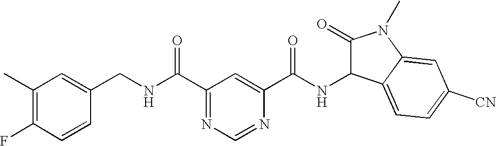 |
| 80 | 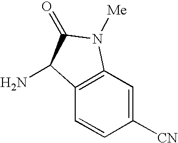 | 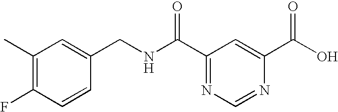 | 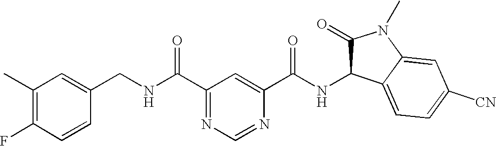 |
| 81 | 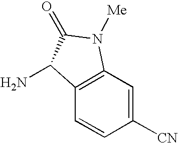 | 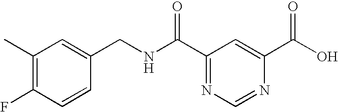 | 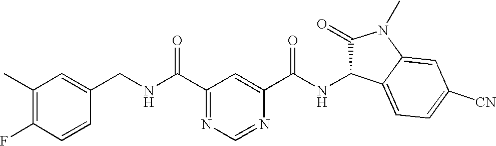 |
| 82 | 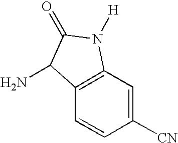 | 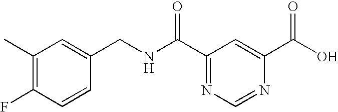 | 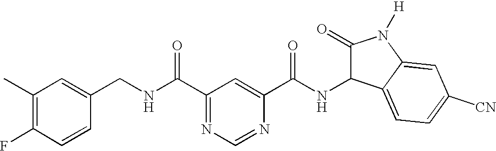 |
| 83 | 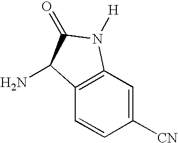 | 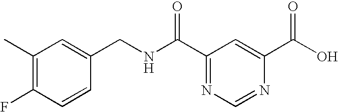 | 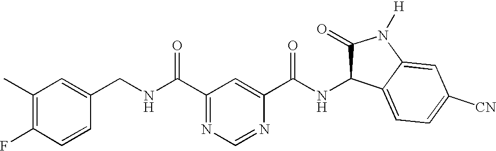 |
| 84 | 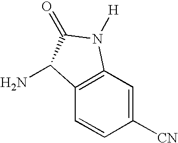 | 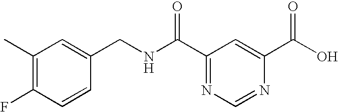 | 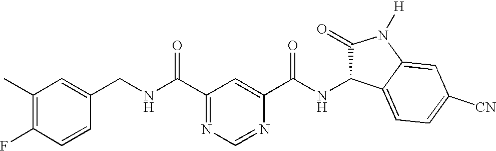 |
| 85 | 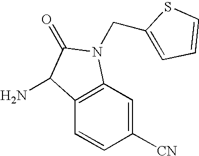 | 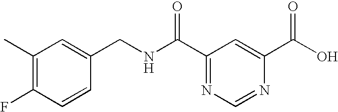 | 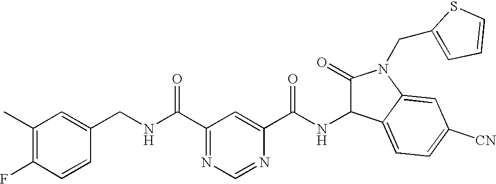 |
| 86 | 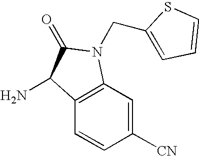 | 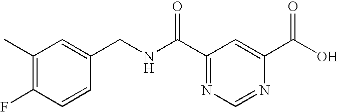 | 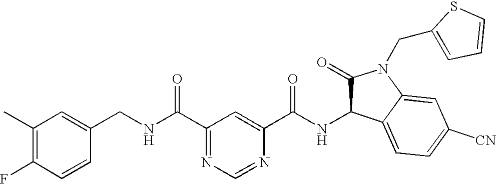 |
| 87 | 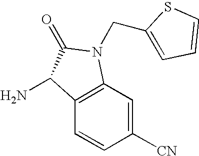 | 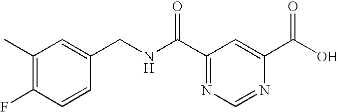 | 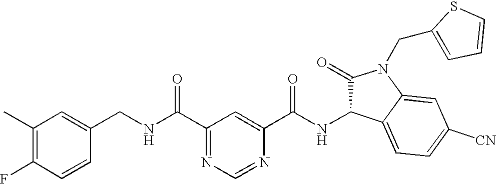 |
| 88 | 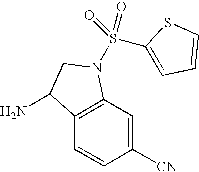 | 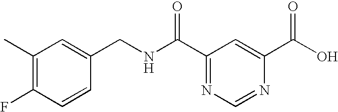 | 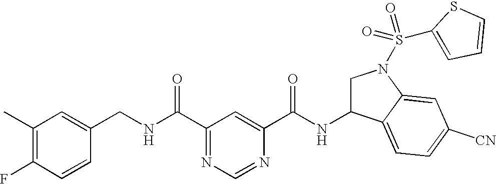 |
| 89 | 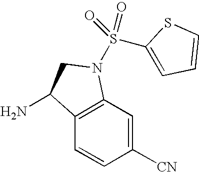 | 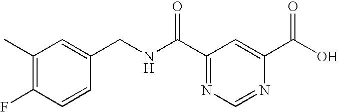 | 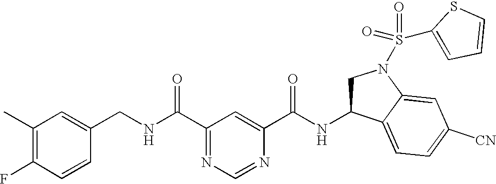 |
| 90 | 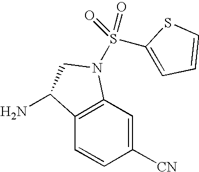 | 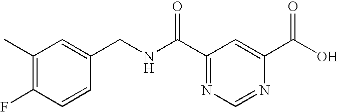 | 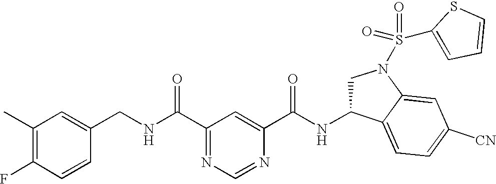 |
| 91 | 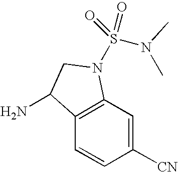 | 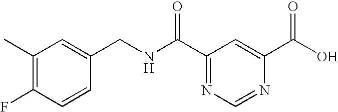 | 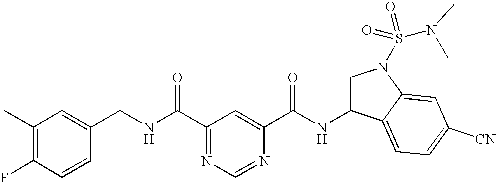 |
| 92 | 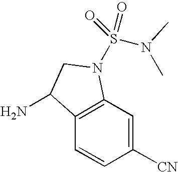 | 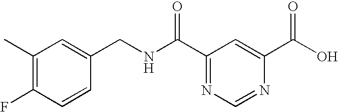 | 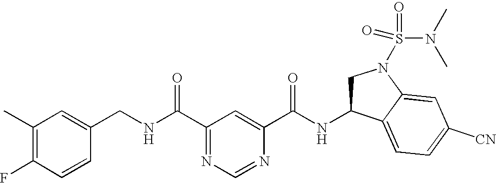 |
| 93 | 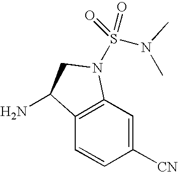 | 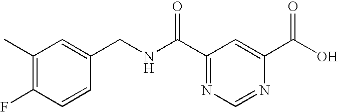 | 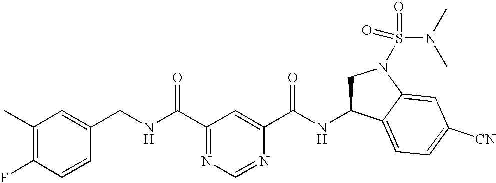 |
| 94 | 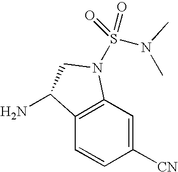 | 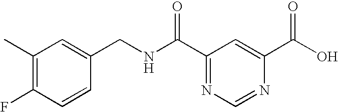 | 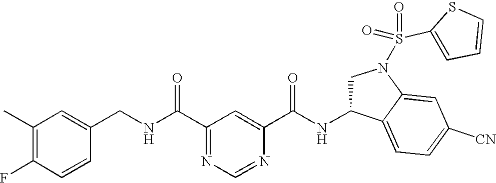 |
| 95 | 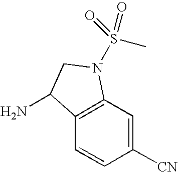 | 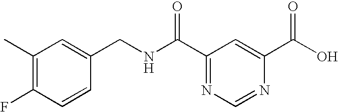 | 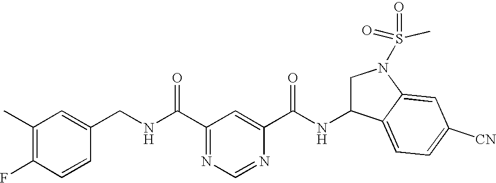 |
| 96 | 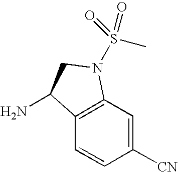 | 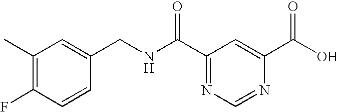 | 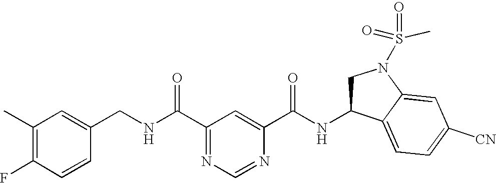 |
| 97 | 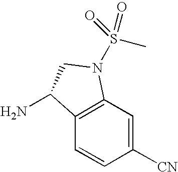 | 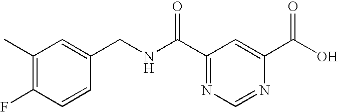 | 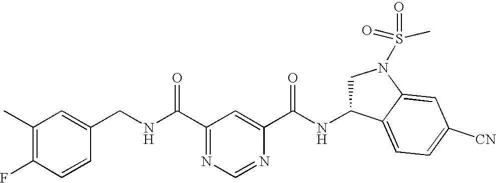 |
| 98 | 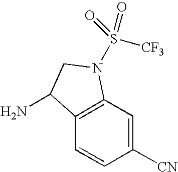 | 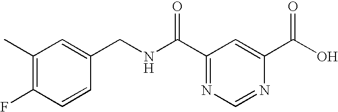 | 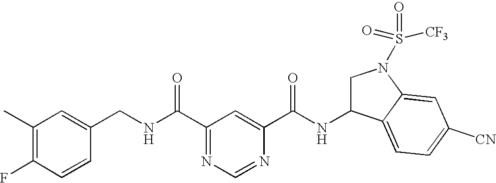 |
| 99 | 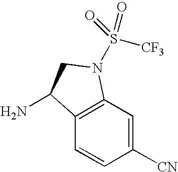 | 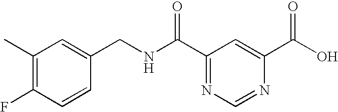 | 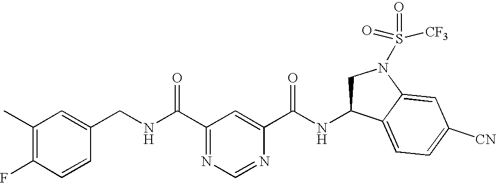 |
| 100 | 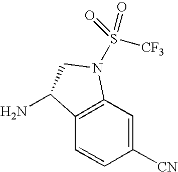 | 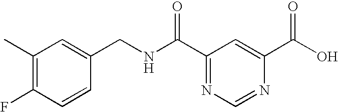 | 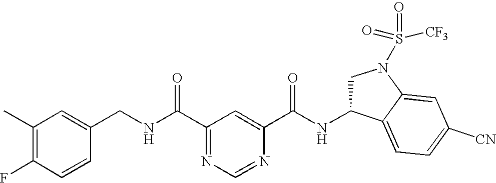 |
| 101 | 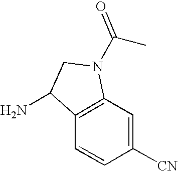 | 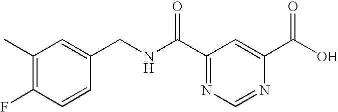 | 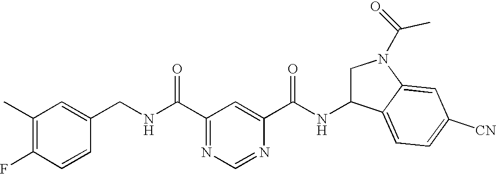 |
| 102 | 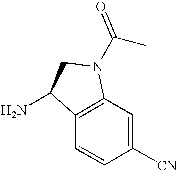 | 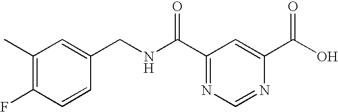 | 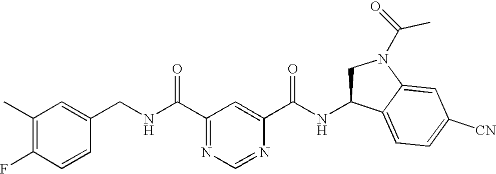 |
| 103 | 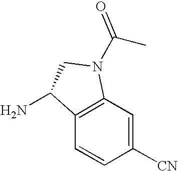 | 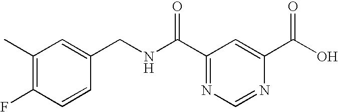 | 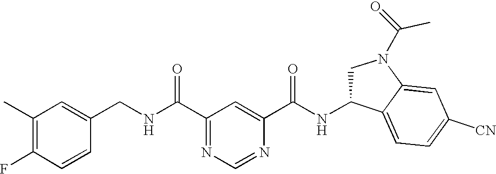 |
| 104 | 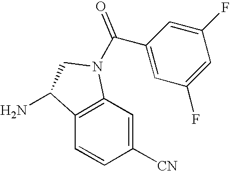 | 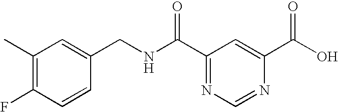 | 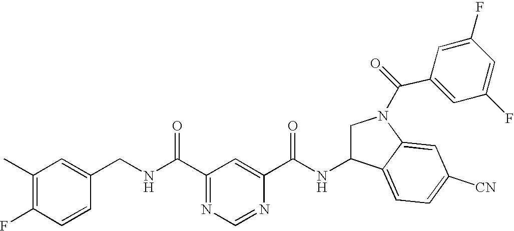 |
| 105 | 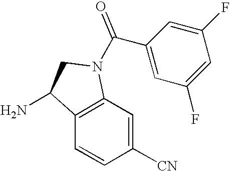 | 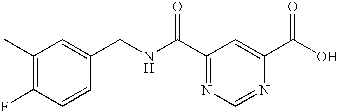 | 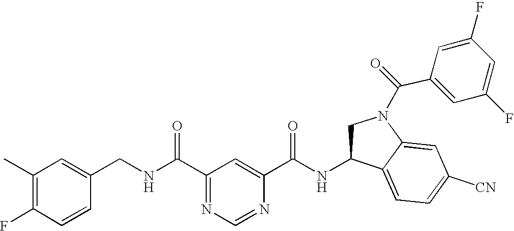 |
| 106 | 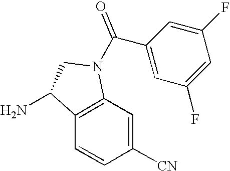 | 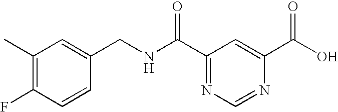 | 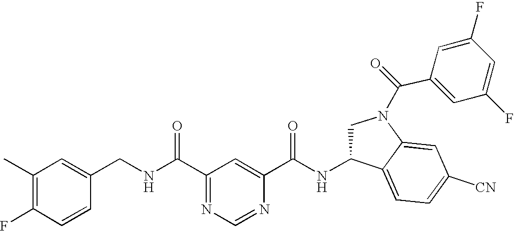 |
| 107 | 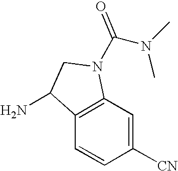 | 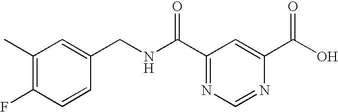 | 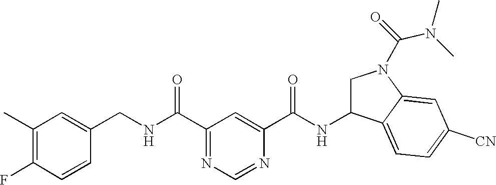 |
| 108 | 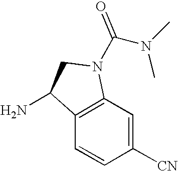 | 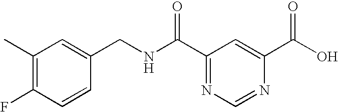 | 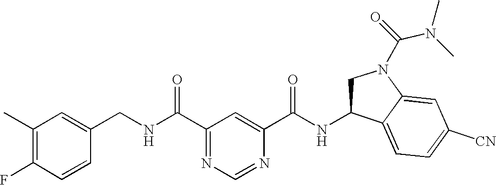 |
| 109 | 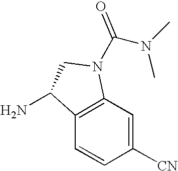 | 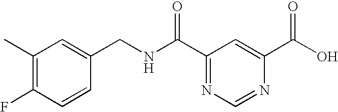 | 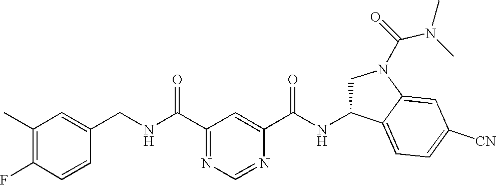 |
| 110 | 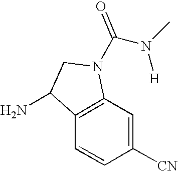 | 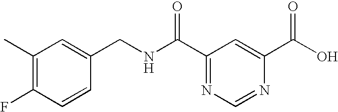 | 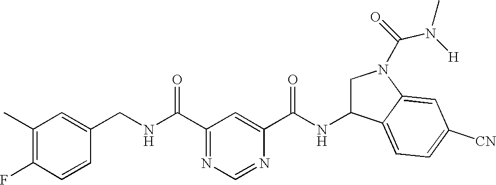 |
| 111 | 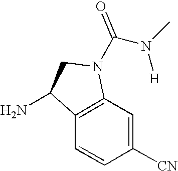 | 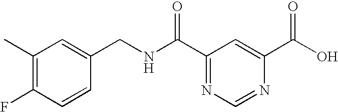 | 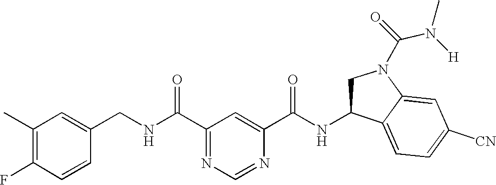 |
| 112 | 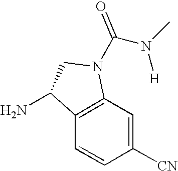 | 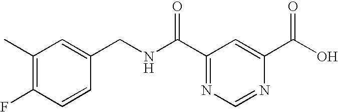 | 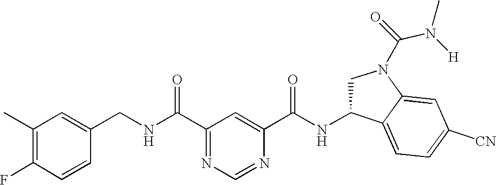 |
| 113 | 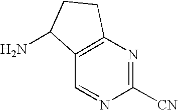 | 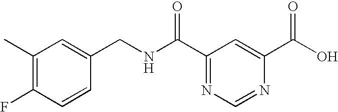 | 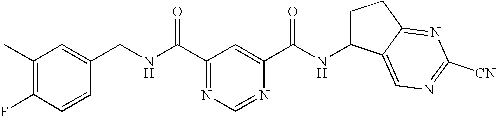 |
| 114 | 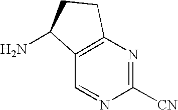 | 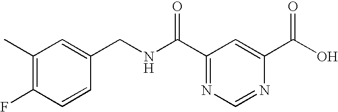 | 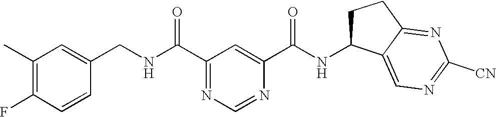 |
| 115 | 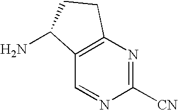 | 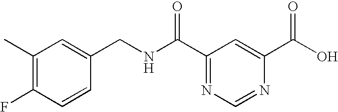 | 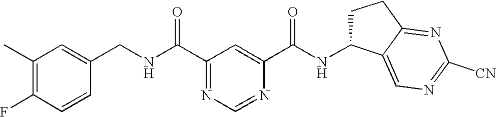 |
| 116 | 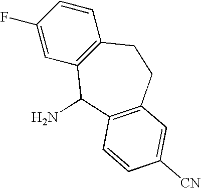 | 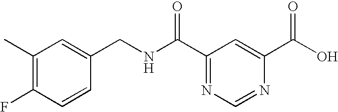 | 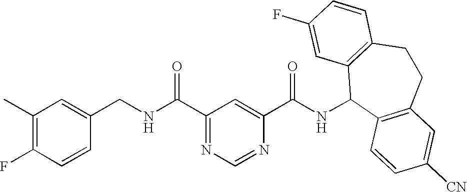 |
| 117 | 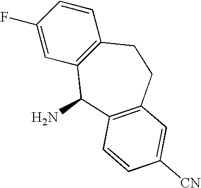 | 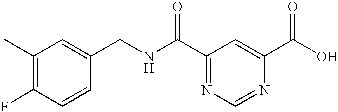 | 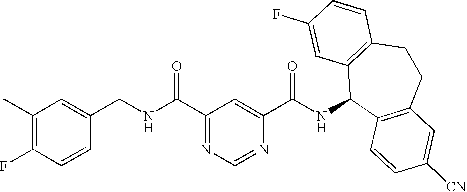 |
| 118 | 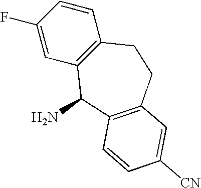 | 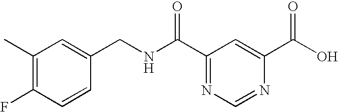 | 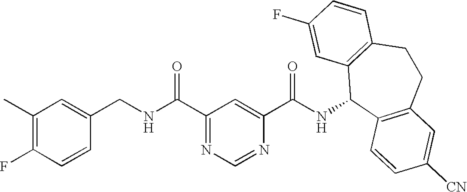 |
| 119 | 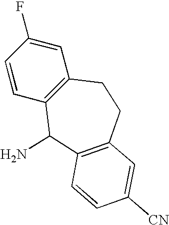 | 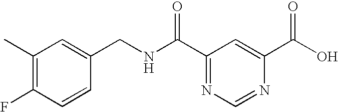 | 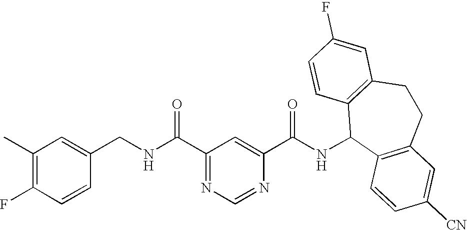 |
| 120 | 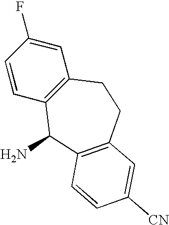 | 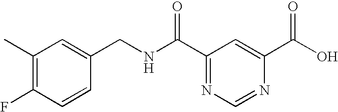 | 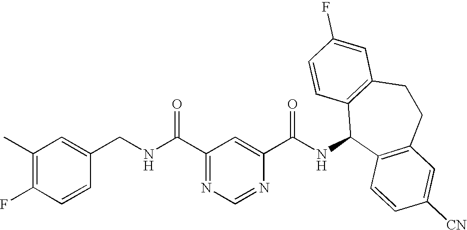 |
| 121 | 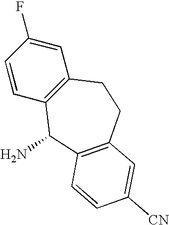 | 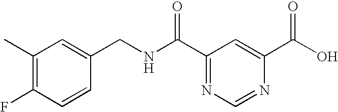 | 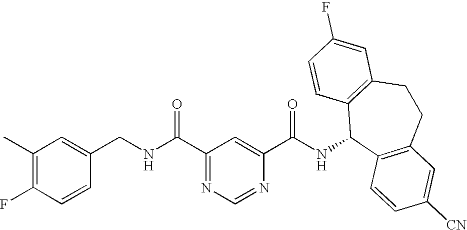 |
| 122 | 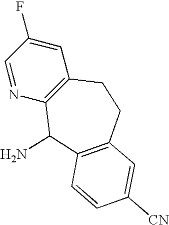 | 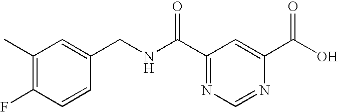 | 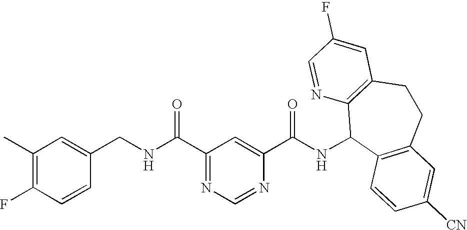 |
| 123 | 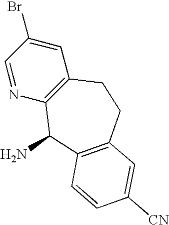 | 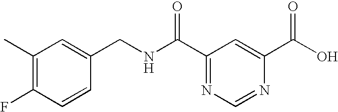 | 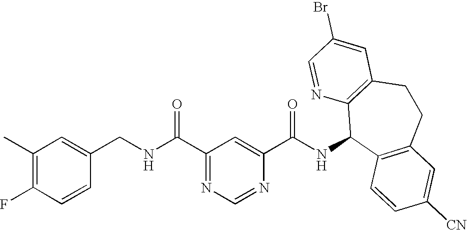 |
| 124 | 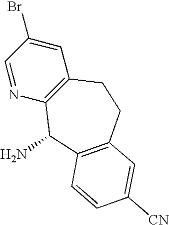 | 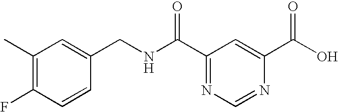 | 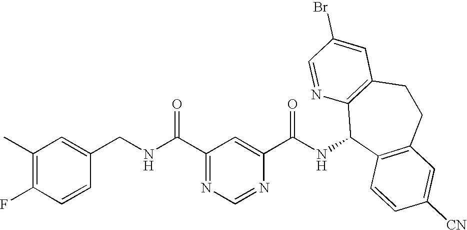 |
| 125 | 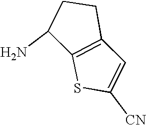 | 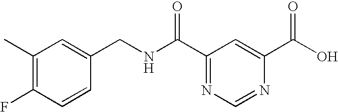 | 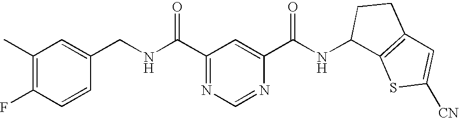 |
| 126 | 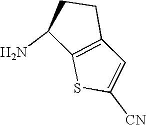 | 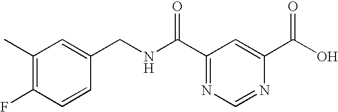 | 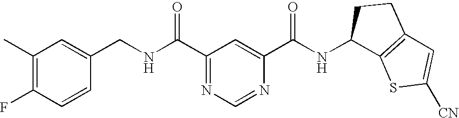 |
| 127 | 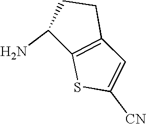 | 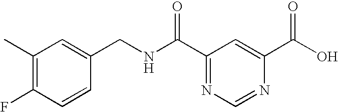 | 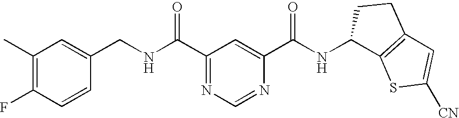 |
| 128 | 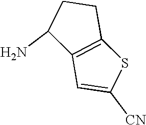 | 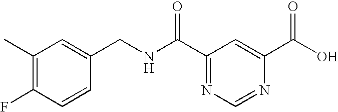 | 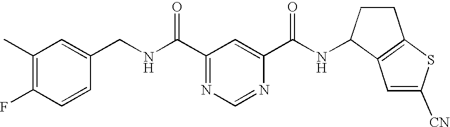 |
| 129 | 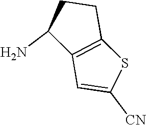 | 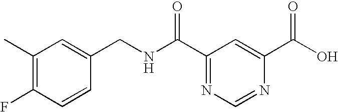 | 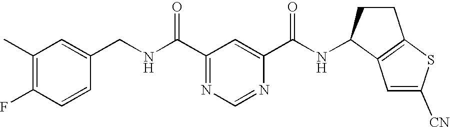 |
| 130 | 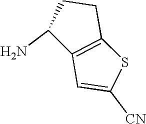 | 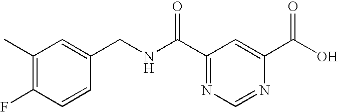 | 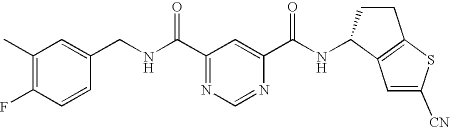 |
| 132 | 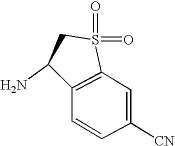 | 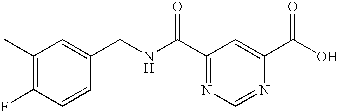 | 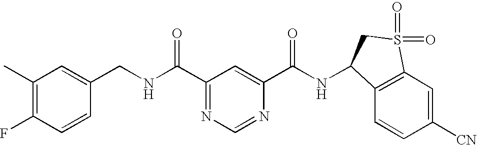 |
| 133 | 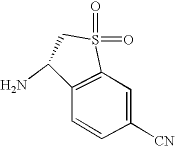 | 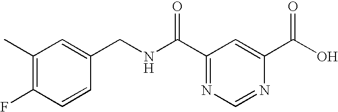 | 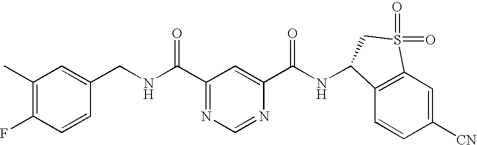 |
| 135 | 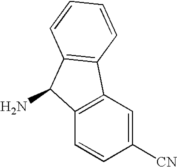 | 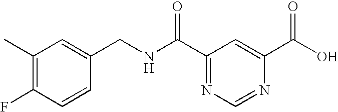 | 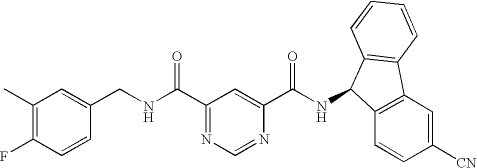 |
| 136 | 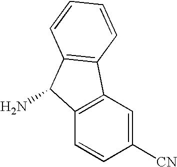 | 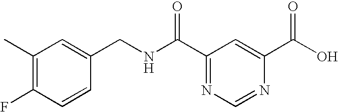 | 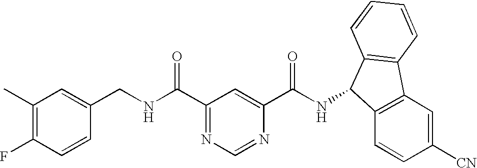 |
| 137 | 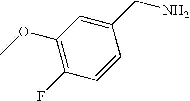 | 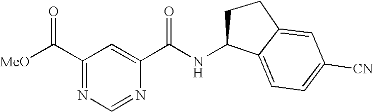 | 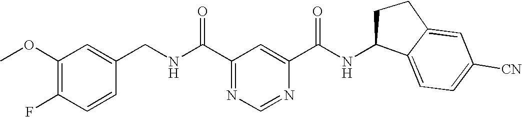 |
| 138 | 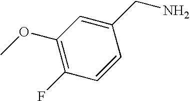 | 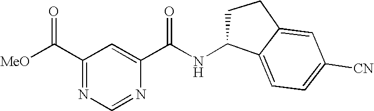 | 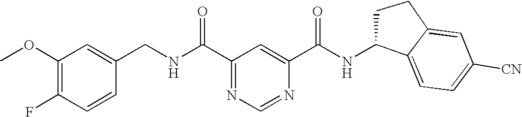 |
| 139 | 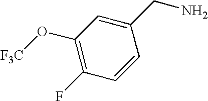 | 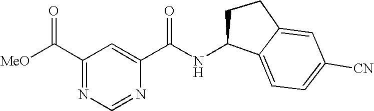 | 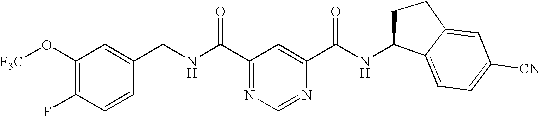 |
| 140 | 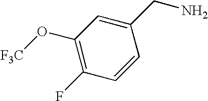 | 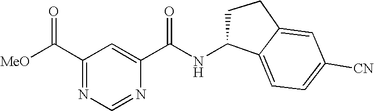 | 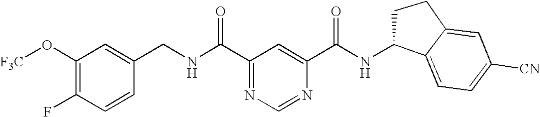 |
| 141 | 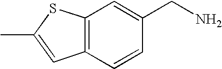 | 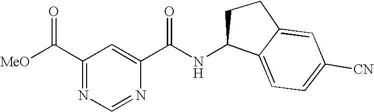 | 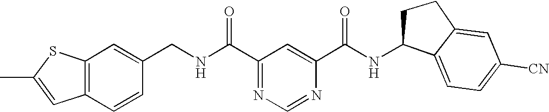 |
| 142 | 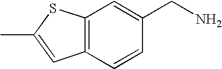 | 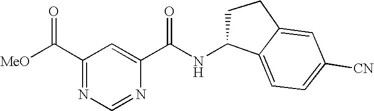 | 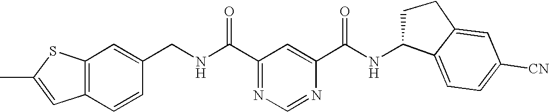 |
| 143 | 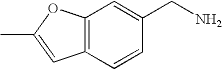 | 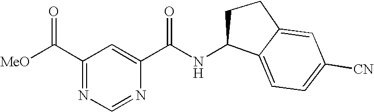 | 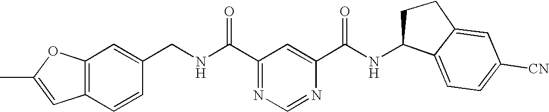 |
| 144 | 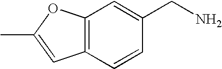 | 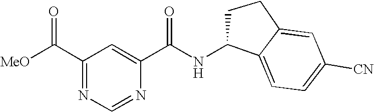 | 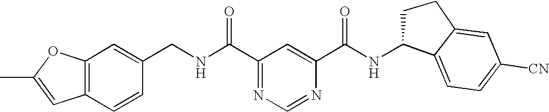 |
| 145 | 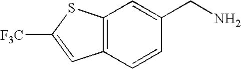 | 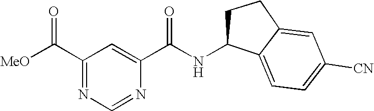 | 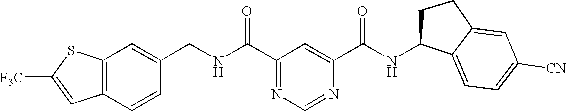 |
| 146 | 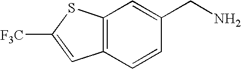 | 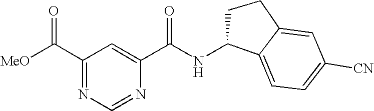 | 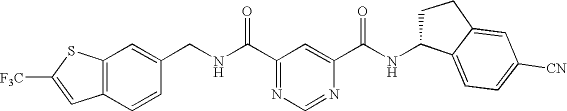 |
| 147 | 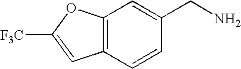 | 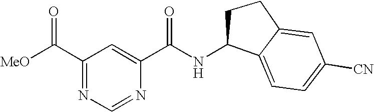 | 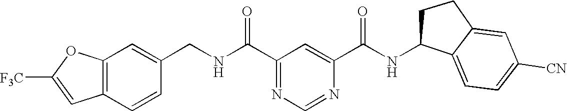 |
| 148 | 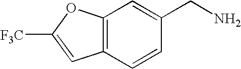 | 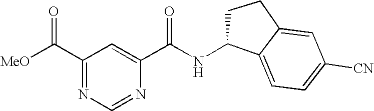 | 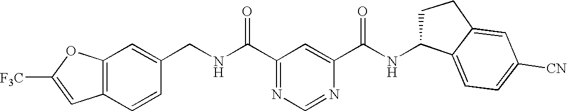 |
| 149 | 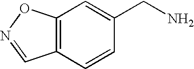 | 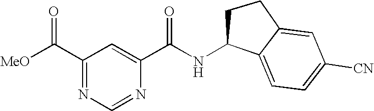 | 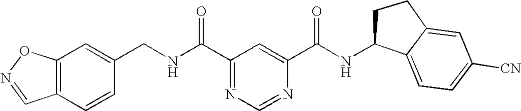 |
| 150 | 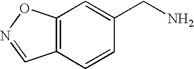 | 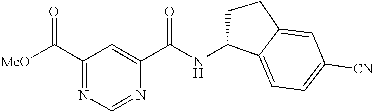 | 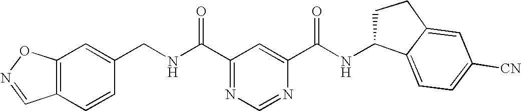 |
| 151 | 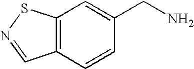 | 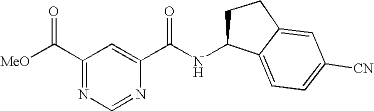 | 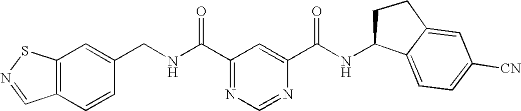 |
| 152 | 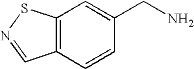 | 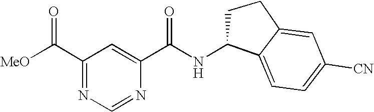 | 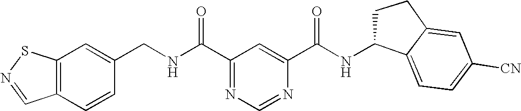 |

### Preparative Example 2001

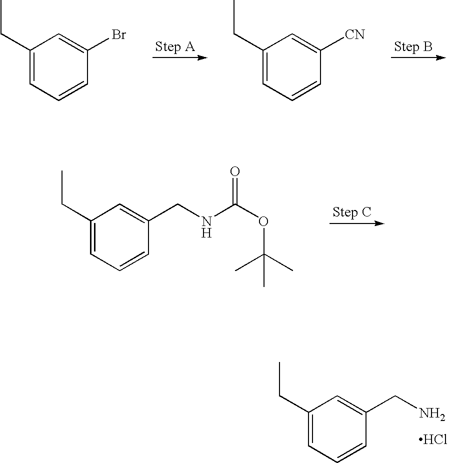 Step A

Commercially available 1-brom-3-ethyl-benzene (1.1 g), zinc cyanide (508 mg), tetrakis-(triphenylphospine)palladium (333 mg) were dissolved in dry toluene (8 mL), degassed and stirred at 80° C. in a sealed pressure tube under argon. After 12 h the mixture was concentrated to dryness. The remaining residues was purified by column chromatography (silica, cyclohexane/EtOAc, 95:5) to afford the title compound (470 mg; 62%). [MH]⁺=132.

Step B

The title compound from Step A above (470 mg), di-tert-butyl dicarbonate (1.56 g) and nickel(II) chloride hexahydrate (85 mg) were dissolved in dry methanol (30 mL) and cooled to 0° C. Then sodium borohydride (948 mg) was added in small portions. The ice bath was removed and the mixture was vigorously stirred for 4 h. Then diethylenetriamine (385 μL) was added and the mixture was concentrated to dryness. The residue was dissolved in ethyl acetate, washed with 10% citric acid, saturated sodium hydrogen carbonate and brine. The organic phase was separated, dried over MgSO₄, filtered and concentrated. The residue was purified by column chromatography (silica, cyclohexane/EtOAc, 95:5 to 9:1) to afford the intermediate as a colourless oil. (341 mg, 40%). [MH]⁺=236.

Step C

A solution of the title compound from Step B above (341 mg) in hydrogen chloride (4M solution in dioxane) was stirred for 1 h at room temperature. The solvent was removed to afford the title compound (250 mg; quantitative). [M-Cl]⁺=136.

### Preparative Examples 2002-2003

Following a similar procedure as that described in Preparative Example 2001, except using the compounds from the Preparative Examples indicated in Table 3 below, the following compounds were prepared.

TABLE 3

| | | | Yield |
|---|---|---|---|
| | Phenyl | | (3 steps) |
| Ex # | bromid | Product | MS |
| 2002 | 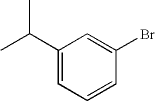 | 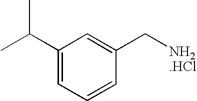 | 34% [M—Cl]⁺ = 150 |
| 2003 | 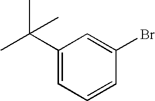 | 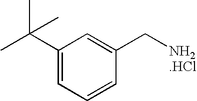 | 24% [M—Cl]⁺ = 164 |

### Preparative Example 2004

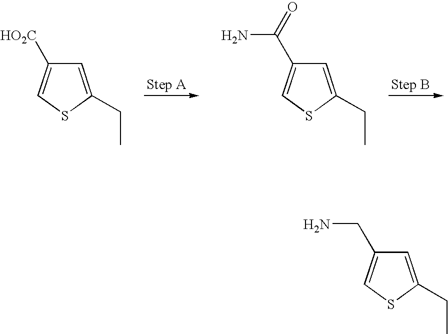 Step A

To commercially available 5-ethyl-thiophene-3-carboxylic acid (3.0 g) in dry methylene chloride (50 mL) at 0° C. was added oxalyl chloride (2.3 mL) followed by DMF (0.4 mL) and the mixture was stirred for 1 h at 0° C., then 3 h at room temperature. The reaction was then concentrated to an oil. The oil was then dissolved in methylene chloride (3 mL) and then slowly added to condensed ammonia (30 mL) at approx. -40° C. The reaction mixture was stirred at approx. −30° C. for 1 h and then allowed to slowly warm up to room temperature (˜10 h). The volatile components of the reaction mixture were removed under reduced pressure to give the intermediate (2.0 g; 68%) as a tan solid. [MH]⁺=156.

Step B

The intermediate from Step A above (1.0 g) and tetrabutylammonium borohydride (4.9 g) in dry methylene chloride (30 mL) was vigorously stirred and heated (55-62° C.) for 24 h and then concentrated to an oil. To the chilled (0° C.) oil was slowly added 1N hydrochloric acid (15 mL) over a period of 1 h. The aqueous mixture was then heated at 100° C. for 1 h, cooled to room temperature, washed with diethyl ether (100 mL), basified with concentrated aqueous KOH to approx. pH 10. The aqueous phase was then extracted with diethyl ether (100 mL) and organic phase separated and dried (MgSO₄), filtered and concentrated to give the title compound (0.25 g; 27%) as an oil. [MH]⁺=142.

### Preparative Example 2005

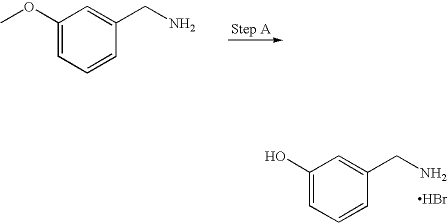 Step A

To a solution of commercially available 3-methoxy-benzylamine (500 mg) in dichloromethane (5 mL) was added BBr₃ (1M in dichloromethane, 7.3 mL) at 0° C. The mixture was stirred for 16 h at rt. Then methanol was added (5 mL) and the mixture was stirred for 2 h and then concentrated to afford the title compound (740 mg, quantitative). ¹H-NMR (CDCl₃) δ=3.90 (br s, 2 H), 6.70-6.85 (m, 3 H), 7.18 (t, 1 H), 8.10 (br s, 3 H).

### Preparative Example 2006

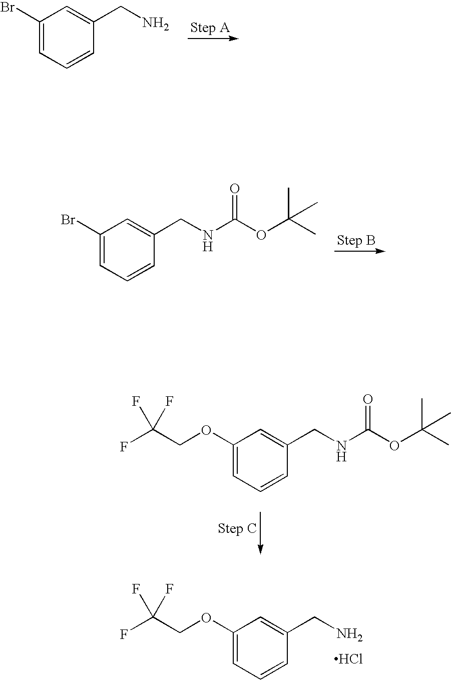 Step A

To a solution of commercially available 3-bromo-benzylamine (938 mg) in dry dichloromethane (10 mL) was added added di-tert-butyl dicarbonate (1.10 g). The resulting clear solution was stirred at room temperature for 15 h and then concentrated to afford the title compound (1.42 g; 99%). [(M-isobutene)H]⁺=230/232, [MNa]⁺=308/310.

Step B

To a suspension of sodium hydride (95%, 303 mg) in dry tetrahydrofurane (10 mL) was carefully added 2,2,2-trifluoroethanol (719 EL). Then copper(I) iodide (2.29 g) and a solution of the title compound from Step A above (572 mg) in dry tetrahydrofurane (2 mL) were added and the resulting suspension was heated to reflux for 17 h. The mixture was cooled to room temperature, diluted with water (20 mL) and methanol (20 mL) and extracted with ethyl acetate (3×50 mL). The combined organic layers were dried (MgSO₄), filtered, concentrated and purified by flash chromatography (silica, cyclohexane/ethyl acetate) to afford the title compound (330 mg; 54%). [(M-isobutene)H]⁺=250, [MNa]⁺=328.

Step C

The title compound from Step B above (305 mg) was dissolved in a 4M solution of hydrochloric acid in dioxane (4 mL). The reaction mixture was stirred at room temperature for 2 h and then concentrated to afford the title compound (239 mg; 99%). [M-l]⁺=206.

### Preparative Example 2007

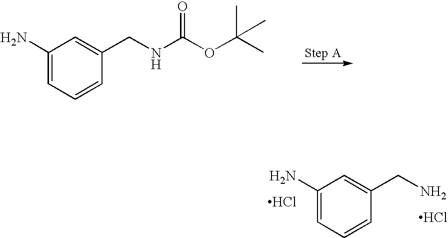 Step A

Commercially available (3-amino-benzyl)-carbamic acid tert-butyl ester (222 mg) was suspended in a 4M solution of hydrochloric acid in dioxane (4 mL). The reaction mixture was stirred at room temperature for 2 h and then concentrated to afford the title compound as the double hydrochloric acid salt (193 mg; 99%). [M-HCI₂]⁺=123.

### Preparative Example 2008

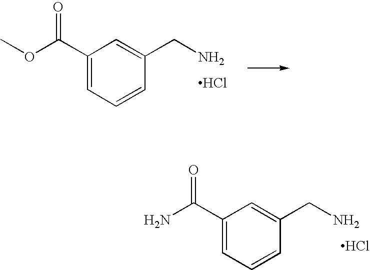

Commercial available 3-aminomethyl-benzoic acid methyl ester hydrochloride (500 mg) was dissolved in aqeous ammonia (33%, 50 mL) and stirred at 90° C. in a sealed pressure tube for 20 h. The solvent was removed to afford the title compound as colorless solid (469 mg; quantitative). [M-Cl]⁺=151.

### Preparative Example 2009

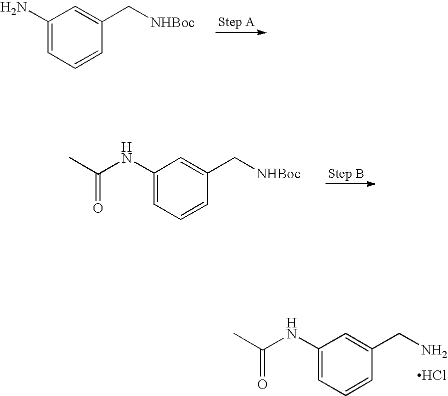 Step A

Commercially available (3-aminobenzyl)-carbamic acid tert-butyl ester (400 mg) was dissolved in pyridine (8 mL), cooled to 0C and acetyl chloride (154 μL) was added. The reaction mixture was allowed to reach room temperature overnight. The mixture was cooled to 0° C., neutralized with 1M hydrochloric acid and diluted with water (15 mL). After extraction with dichloromethane (3×50 mL), the organic layer was collected, dried (MgSO₄), concentrated and purified by column chromatography (silica, cyclohexane/EtOAc, 1: 1) to afford the intermediate (333 mg; 70%) as a pale yellow oil. [MNa]⁺=287.

Step B

To the intermediate from Step A above (333 mg) was added hydrogen chloride (4M in dioxane, 5 mL) and the suspension was stirred at room temperature for 1 h. The reaction mixture was evaporated to afford the title compound as colourless solid (251 mg; quantitative). [M-Cl]⁺=165.

### Preparative Example 2010

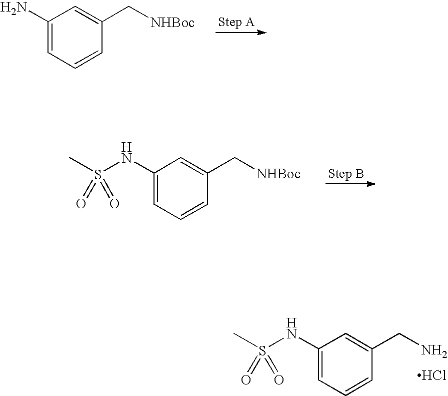 Step A

Commercially available (3-aminobenzyl)-carbamic acid tert-butyl ester (400 mg) was dissolved in pyridine (5 mL) and cooled to 0° C. At this temperature, methanesulfonyl chloride (170 μL) was added and the mixture was allowed to reach room temperature overnight. The reaction mixture was then cooled to 0° C. and carefully neutralized with 1M hydrochloric acid and diluted with water. The aqueous layer was extracted with dichloromethane. The combined organic layer was washed with water and brine, dried (MgSO₄), concentrated and purified by column chromatography (silica, cyclohexane/EtOAc, 1:1) to afford the intermediate (407 mg; 75%) as colourless crystals. [MNa]⁺=323.

Step B

To the intermediate from Step A above (407 mg) was added hydrogen chloride (4M in dioxane, 5 mL) and the suspension was stirred at room temperature for 1 h. The reaction mixture was evaporated to afford the title compound as a colourless solid (350 mg; quantitative). [M-NH₃Cl]⁺=184.

### Preparative Example 2011

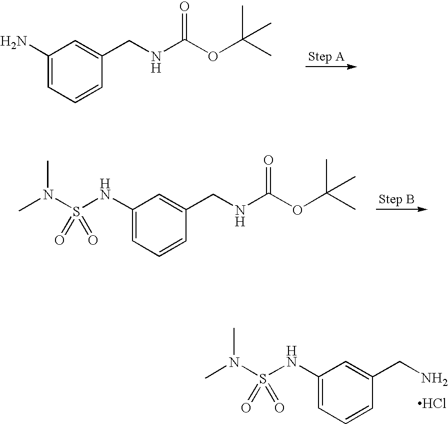 Step A

To a solution of commercially available (3-amino-benzyl)-carbamic acid tert-butyl ester (222 mg) in dry pyridine (1 mL) was added N,N-dimethylsulfamoyl chloride (110 μL). The resulting dark red reaction mixture was stirred at room temperature for 67 h and then diluted with water (10 mL) and ethyl acetate (20 mL). The organic layer was separated and washed with 1M aqueous ammonium chloride (2×10 mL). The aqueous layer were combined and extracted with ethyl acetate (2×10 mL). The combined organic layer were dried (MgSO₄), filtered and concentrated. The remaining residue was purified by flash chromatography (silica, dichloromethane/methanol) to afford the title compound (248 mg; 75%). [(M-isobutene)H]⁺=274, [MH]⁺=330.

Step B

A 4M solution of hydrochloric acid in dioxane (2.8 mL) was added to a solution of the title compound from Step A above (231 mg) in methanol (1.4 mL). The reaction mixture was stirred at room temperature for 2 h and then concentrated to afford the title compound (184 mg; 99%). [M-Cl]⁺=230.

### Preparative Example 2012

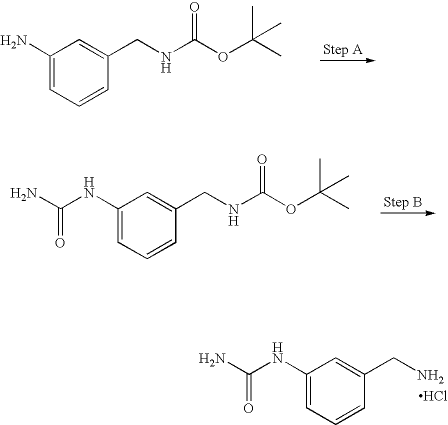 Step A

To a solution of commercially available (3-amino-benzyl)-carbamic acid tert- butyl ester (222 mg) in dry dichloromethane (1 mL) were successively added isopropanol (100 μL) and trimethylsilyl isocyanate (279 μL). The resulting reaction mixture was stirred at room temperature for 68 h, then diluted with methanol (5 mL) and concentrated. The remaining solid was washed with dichloromethane (3×20 mL), dissolved in methanol (20 mL) and concentrated to afford the title compound as a colourless solid (187 mg; 70%). [MH]⁺=266, [MNa]⁺=288.

Step B

A 4M solution of hydrochloric acid in dioxane (2 mL) was added to a solution of title compound from Step A above (133 mg) in methanol (1 mL). The reaction mixture was stirred at room temperature for 2 h and then concentrated to afford the title compound (100 mg; 99%). [M-Cl]⁺=166.

### Preparative Example 2013

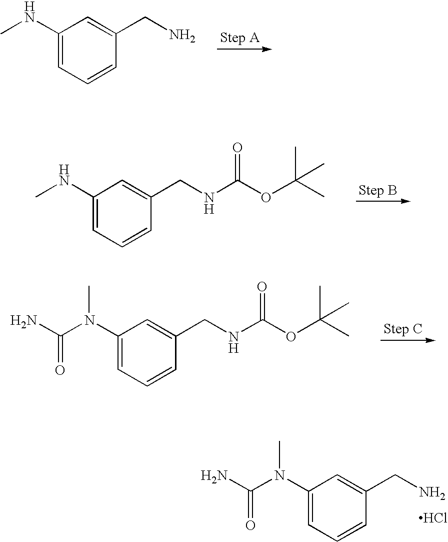 Step A

To a solution of commercially available (3-aminomethyl-phenyl)-methylamine (1.84 g) in dry tetrahydrofurane (40 mL) was added di-tert-butyl dicarbonate (2.95 g). The mixture was stirred at room temperature overnight and concentrated. The remaining residue was dissolved in tert-butyl methyl ether and washed with saturated aqueous sodium hydrogen carbonate and brine, dried (MgSO₄), filtered and concentrated. The remaining residue was purified by flash chromatography (silica, cyclohexane/ethyl acetate) to afford the title compound (3.19 g; >99%). [MH]⁺=237.

Step B

To a solution of title compound from Step A above (709 mg) in dry dichloromethane (3 mL) were successively added isopropanol (300 μL) and trimethylsilyl isocyanate (836 μL). The resulting reaction mixture was stirred at room temperature for 46 h, then diluted with methanol (15 mL) and concentrated. The remaining residue was purified by flash chromatography (silica, dichloromethane/methanol) to afford the title compound (683 mg; 82%). [MH]⁺=280, [MNa]⁺=302.

Step C

A 4M solution of hydrochloric acid in dioxane (9.6 mL) was added to a solution of title compound from Step B above (672 mg) in methanol (4.8 mL). The reaction mixture was stirred at room temperature for 2 h and then concentrated to afford the title compound (512 mg; 99%). [MH]⁺=180.

### Preparative Example 2014

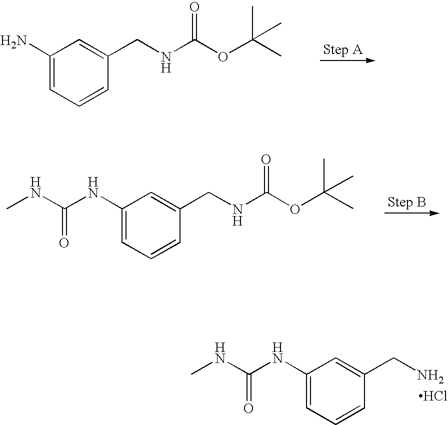 Step A

To a solution of commercially available (3-amino-benzyl)-carbamic acid tert- butyl ester (222 mg) in dry dichloromethane (1 mL) were successively added ethyl diisopropyl amine (349 μL) and N-succinimidyl N-methylcarbamate (355 mg). The resulting reaction mixture was stirred at room temperature for 72 h, then diluted with ethyl acetate (20 mL) and washed with 0.1 M aqueous sodium hydroxide (3×10 mL). The combined organic layer were dried (MgSO₄), filtered and concentrated. The remaining residue was purified by flash chromatography (silica, dichloromethane/methanol) to afford the title compound (223 mg; 80%). [MH]⁺=280, [MNa]⁺=302.

Step B

A 4M solution of hydrochloric acid in dioxane (2 mL) was added to a suspension of title compound from Step A above (140 mg) in methanol (1 mL). The reaction mixture was stirred at room temperature for 2 h and then concentrated to afford the title compound as the hydrochloric acid salt (106 mg; 99%). [M-Cl]⁺=180, [MNa-HCl]⁺=202.

### Preparative Example 2015

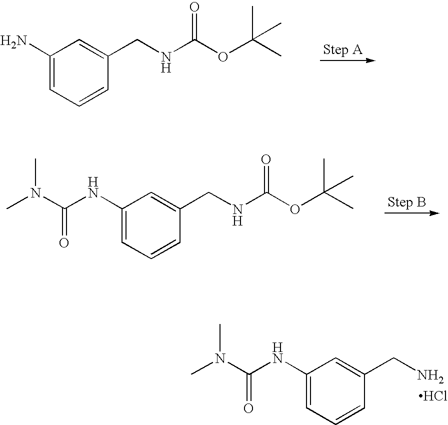 Step A

To a solution of commercially available (3-amino-benzyl)-carbamic acid tert- butyl ester (222 mg) in dry pyridine (1 mL) was added N,N-dimethylcarbamoyl chloride (103 μL). The resulting dark red reaction mixture was stirred at room temperature for 67 h and then diluted with water (10 mL) and ethyl acetate (20 mL). The organic layer was separated and washed with 1M aqueous ammonium chloride (2×10 mL). The aqueous layer were combined and extracted with ethyl acetate (2×10 mL). The combined organic layer was dried (MgSO₄), filtered and concentrated to afford the title compound (241 mg; 82%). [(M-Boc)H]⁺=194, [(M-isobutene)H]⁺=238.

Step B

A 4M solution of hydrochloric acid in dioxane (2.8 mL) was added to a solution of title compound from Step A above (205 mg) in methanol (1.4 mL). The reaction mixture was stirred at room temperature for 2 h and then concentrated to afford the title compound (159 mg; 99%). [M-Cl]⁺=194.

### Preparative Example 2016

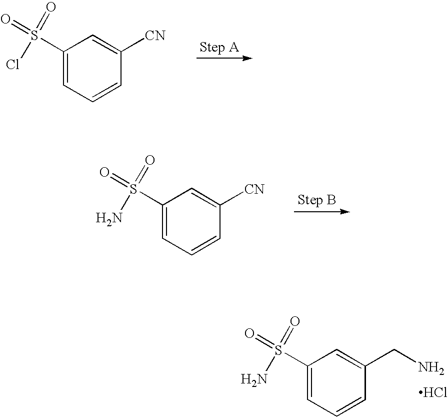 Step A

A solution of 3-cyano-benzenesulfonyl chloride (1.07 g) in ammonia (33% aqueous solution, 40 mL) was stirred for 1 h and then evaporated under reduced pressure to approx. 20 mL and cooled. The precipitate was filtered and washed with water and dried in vaccuo to afford the intermediate (722 mg; 75%) as a colourless solid. [MH]⁺=183.

Step B

The intermediate from step A above (722 mg), di-tert-butyl dicarbonate (1.6 g) and nickel(II) chloride hexahydrate (80 mg) was dissolved in dry methanol (20 mL) and cooled to 0° C. Then sodium borohydride (1.0 g) was added in portions and the ice bath removed. The mixture was vigorously stirred for 2 h, then diethylenetriamine (300 μL) was added and the mixture was concentrated to dryness. The residue was diluted with ethyl acetate, washed with 10% citric acid, saturated sodium hydrogen carbonate and brine, dried (MgSO₄) and concentrated. Purification by column chromatography (dichloromethane/methanol, 96:4 to 95:5) gave a amorphous mass, which was suspended in hydrogen chloride (4M solution in dioxane, 15 mL) and stirred for 6 h, evaporated, slurried in diethyl ether and filtered to afford the title compound (590 mg; 67%). [M-Cl]⁺=187.

### Preparative Example 2017

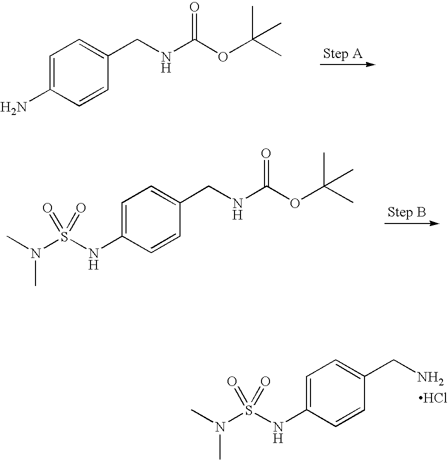 Step A

To a solution of commercially available (4-amino-benzyl)-carbamic acid tert- butyl ester (229 mg) in dry pyridine (1 mL) was added N,N-dimethylsulfamoyl chloride (110 μL). The resulting dark red reaction mixture was stirred at room temperature for 67 h and then diluted with water (10 mL) and ethyl acetate (20 mL). The organic layer was separated and washed with 1M aqueous ammonium chloride (2×10 mL). The aqueous layer were combined and extracted with ethyl acetate (2×10 mL). The combined organic layer were dried (MgSO₄), filtered and concentrated. The remaining residue was purified by flash chromatography (silica, dichloromethane/methanol) to afford the title compound (269 mg; 82%). [(M-isobutene)H]⁺=274.

Step B

A 4M solution of hydrochloric acid in dioxane (2.8 mL) was added to a solution of title compound from Step A above (231 mg) in methanol (1.4 mL). The reaction mixture was stirred at room temperature for 2 h and then concentrated to afford the title compound (184 mg; 99%). [M-NH₃Cl]⁺=213.

### Preparative Example 2018

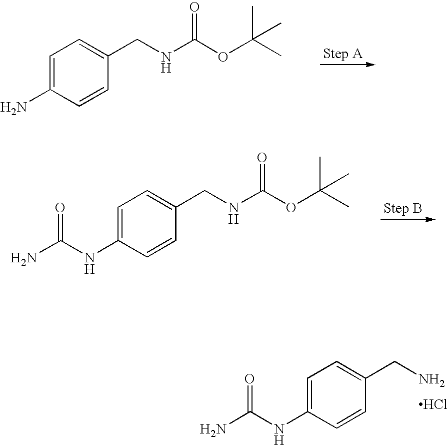 Step A

To a solution of commercially available (4-amino-benzyl)-carbamic acid tert- butyl ester (229 mg) in dry dichloromethane (1 mL) were successively added isopropanol (100 μL) and trimethylsilyl isocyanate (154 μL). The resulting reaction mixture was stirred at room temperature for 17½ h. Additional trimethylsilyl isocyanate (154 μL) was added and stirring at room temperature was continued for 75 h. The resulting reaction mixture was diluted with methanol (5 mL) and concentrated. The remaining residue was purified by flash chromatography (silica, dichloromethane/methanol) to afford the title compound (263 mg; 99%). [MH]⁺=266, [MNa]⁺=288.

Step B

The title compound from Step A above (186 mg) was dissolved in a 4M solution of hydrochloric acid in dioxane (2.8 mL) The reaction mixture was stirred at room temperature for 1½ h and then concentrated to afford the title compound (139 mg; 99%). [M-Cl]⁺=166.

### Preparative Example 2019

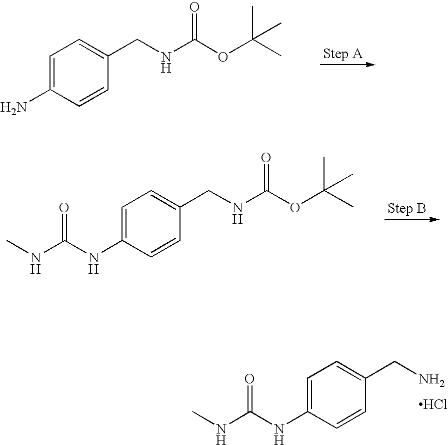 Step A

To a solution of commercially available (4-amino-benzyl)-carbamic acid tert- butyl ester (229 mg) in dry dichloromethane (1 mL) were successively added ethyl diisopropyl amine (349 μL) and N-succinimidyl N-methylcarbamate (355 mg). The resulting reaction mixture was stirred at room temperature for 72 h, then diluted with ethyl acetate (20 mL) and washed with 0.1 M aqueous sodium hydroxide (3×10 mL). The combined organic layer were dried (MgSO₄), filtered and concentrated to afford the title compound (269 mg; 96%). [MH]⁺=280, [MNa]⁺=302.

Step B

A 4M solution of hydrochloric acid in dioxane (2.8 mL) was added to a suspension of title compound from Step A above (196 mg) in methanol (1.4 mL). The reaction mixture was stirred at room temperature for 2 h and then concentrated to afford the title compound (149 mg; 99%). [M-Cl]⁺=180, [MNa—HCl]⁺=202.

### Preparative Example 2020

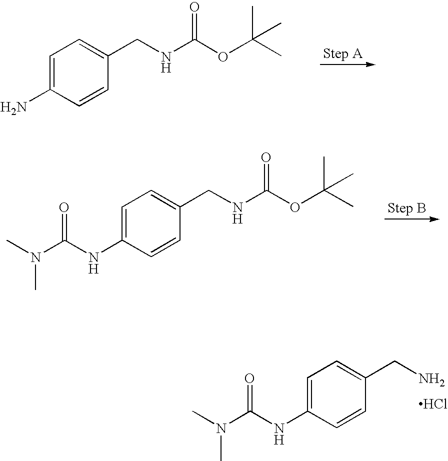 Step A

To a solution of commercially available (4-amino-benzyl)-carbamic acid tert-butyl ester (222 mg) in dry pyridine (1 mL) was added N,N-dimethylcarbamoyl chloride (103 μL). The resulting dark red reaction mixture was stirred at room temperature for 17½ h and then diluted with water (10 mL) and ethyl acetate (20 mL). The organic layer was separated and washed with 1M aqueous ammonium chloride (2×10 mL). The aqueous layer were combined and extracted with ethyl acetate (2×10 mL). The combined organic layer were dried (MgSO₄), filtered and concentrated to afford the title compound (284 mg; 97%). [MH]⁺=294, [MNa]⁺=316.

Step B

A 4M solution of hydrochloric acid in dioxane (2.8 mL) was added to a solution of title compound from Step A above (205 mg) in methanol (1.4 mL). The reaction mixture was stirred at room temperature for 1½ h and then concentrated to afford the title compound (159 mg; 99%). [M-Cl]⁺=194.

### Preparative Example 2021

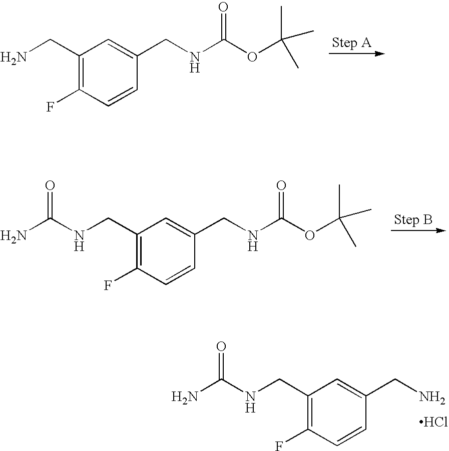 Step A

To a solution of (3-aminomethyl-4-fluorobenzyl) carbamic acid tert-butyl ester (1.63 g) in dry dichloromethane (20 mL) and iso-propanol (2 mL) was added trimethylsilyl isocyanate (1.9 mL) and the mixture was stirred overnight. The solution was concentrated, absorbed on silica and purified by column chromatography (dichloromethane/methanol, 97:3 to 9:1) to afford the intermediate (1.43 g; 68%) as a colourless solid.

Step B

To intermediate from step A above (1.43 g) was added hydrogen chloride (4M solution in dioxane, 20 mL) and stirred for 2.5 h, evaporated, suspended in diethyl ether, filtered and dried to afford the title compound (1.21 g; quantitative) as an off-white solid. [M-NH₃Cl]⁺=180.9, [M-Cl]⁺=197.9.

### Preparative Example 2022

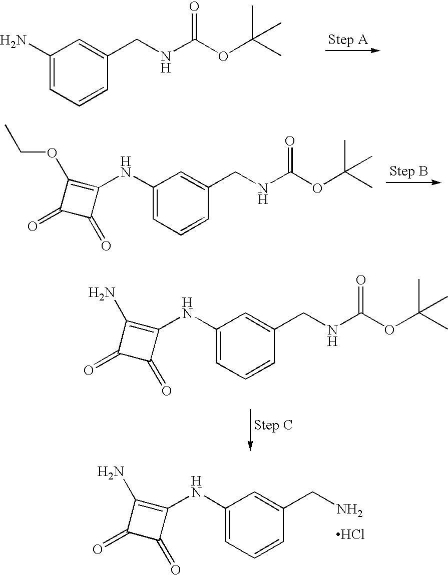 Step A

To a solution of commercially available (3-amino-benzyl)-carbamic acid tert-butyl ester (1.11 g) in ethanol (20 mL) was added 3,4-diethoxy-3-cyclobutene-1,2-dione (1.30 g). The resulting clear solution was heated to reflux for 2½ h. The mixture was cooled to room temperature and the formed solids were removed by filtration. The filtrate was concentrated and the remaining solid residue was crystallized from refluxing ethanol to afford the title compound (687 mg; 40%). [(M-Boc)H]⁺=247, [MNa]⁺=369.

Step B

The title compound from Step A above (346 mg) was dissolved in a ˜7N solution of ammonia in methanol (14.3 mL). The reaction mixture was stirred at room temperature for 3 h and then concentrated to afford the title compound (316 mg; 99%). [(M-Boc)H]⁺=218, [MNa]⁺=340.

Step C

A 4M solution of hydrochloric acid in dioxane (4 mL) was added to a suspension of the crude title compound from Step B above (312 mg) in methanol (2 mL). The reaction mixture was stirred at room temperature for 2 h and then concentrated to afford the title compound (250 mg; 99%). [M-NH₃Cl]⁺=218.

### Preparative Example 2023

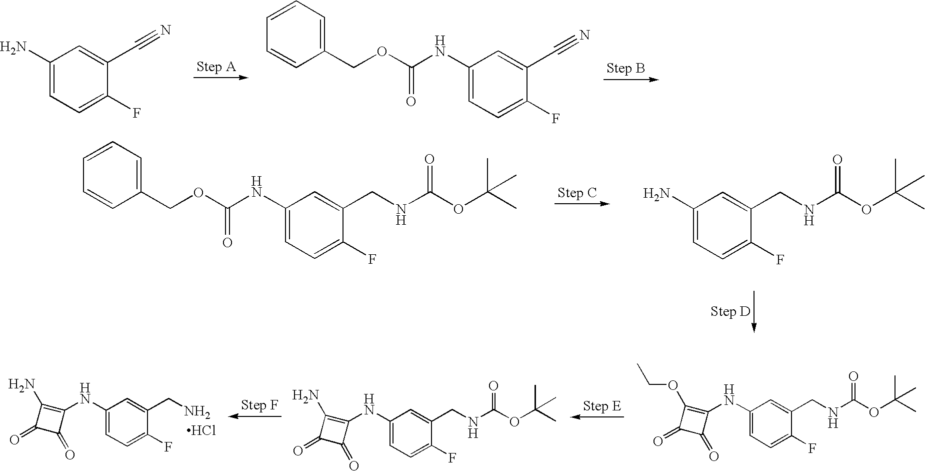 Step A

To a solution of commercially available 5-amino-2-fluoro-benzonitrile (953 mg) in dry tetrahydrofurane (70 mL) were added benzyl chloroformate (1.20 mL) and potassium carbonate (1.16 g). The resulting suspension was stirred at room temperature for 16 h. Additional benzyl chloroformate (1.20 mL) and potassium carbonate (1.16 g) were added and stirring at room temperature was continued for 7 h. The mixture was diluted with ethyl acetate (70 mL), washed with water (2×70 mL), dried (MgSO₄), filtered and concentrated. The remaining residue was purified by flash chromatography (silica, cyclohexane/ethyl acetate) to afford the title compound (1.47 g; 78%). [MH]⁺=271.

Step B

To an ice cooled (0-5° C.) solution of the title compound from Step A above (1.35 g) in dry methanol (50 mL) were added di-tert-butyl dicarbonate (2.23 g) and nickel(II) chloride hexahydrate (123 mg), followed by the careful portion wise addition of sodium borohydride (1.34 g). The resulting black mixture was stirred for 15 min at 0-5 ° C. (ice bath), then the ice bath was removed and stirring was continued for 15 h at room temperature. Then diethylenetriamine (543 μL) was added and stirring at room temperature was continued for 15 min. The mixture was concentrated to dryness, ethyl acetate (50 mL) was added and the resulting suspension was washed with 1M aqueous ammonium chloride solution (50 mL), saturated aqueous sodium hydrogen carbonate (50 mL) and brine (50 mL), dried (MgSO₄), filtered and concentrated. The remaining residue was purified by flash chromatography (silica, cyclohexane/ethyl acetate) to afford the title compound (992 mg; 53%). [(M-Boc)H]⁺=275, [MNa]⁺=397.

Step C

To a solution of the title compound from Step B above (936 mg) in dry methanol (50 mL) was added palladium on charcoal (10 wt %, 266 mg). The resulting black mixture was degassed by three pump/vent with hydrogen and then stirred at room temperature under a hydrogen atmosphere at normal pressure for 17 h. Filtration through a plug of Celite®, concentration and purification by flash chromatography (silica, cyclohexane/ethyl acetate) afforded the title compound (534 mg; 89%). [(M-Boc)H]⁺=141, [MNa]⁺=263.

Step D

To a solution of the title compound from Step C above (240 mg) in ethanol (4 mL) was added 3,4-diethoxy-3-cyclobutene-1,2-dione (261 mg). The resulting clear solution was heated to reflux for 14 h. and then concentrated. The remaining residue was purified by flash chromatography (silica, dichloromethane/methanol) to afford the title compound (245 mg; 67%). [(M-Boc)H]⁺=265, [MNa]⁺=387.

Step E

The title compound from Step D above (219 mg) was dissolved in a ˜7N solution of ammonia in methanol (8.6 mL). The reaction mixture was stirred at room temperature for 16 h and then concentrated to afford the title compound (194 mg; 96%). [(M-Boc)H]⁺=236, [MNa]⁺=358.

Step F

A 4M solution of hydrochloric acid in dioxane (2.2 mL) was added to a suspension of the crude title compound from Step E above (184 mg) in dry methanol (2.2 mL). The reaction mixture was stirred at room temperature for 1 h and then concentrated to afford the title compound (149 mg; 99%). [M-Cl]⁺=236.

### Preparative Example 2024

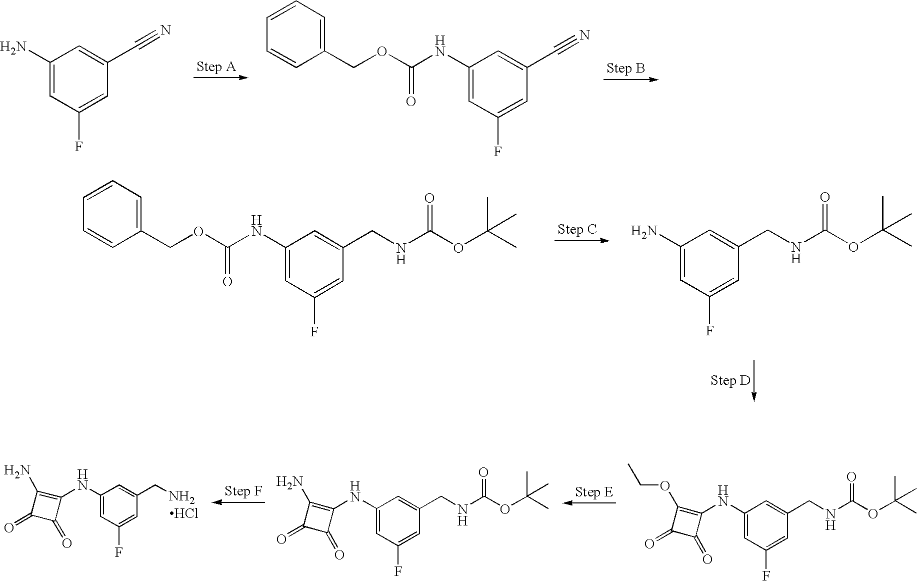

To a solution of commercially available 3-amino-5-fluoro-benzonitrile (953 mg) in dry tetrahydrofurane (70 mL) were added benzyl chloroformate (1.20 mL) and potassium carbonate (1.16 g). The resulting suspension was stirred at room temperature for 16 h. Additional benzyl chloroformate (1.20 mL) and potassium carbonate (1.16 g) were added and stirring at room temperature was continued for 7 h. The mixture was diluted with ethyl acetate (70 mL), washed with water (2×70 mL), dried (MgSO₄), filtered and concentrated. The remaining residue was purified by flash chromatography (silica, cyclohexane/ethyl acetate) to afford the title compound (1.76 g; 93%). [MH]⁺=271.

Step B

To an ice cooled (0-5° C.) solution of the title compound from Step A above (1.62 g) in dry methanol (60 mL) were added di-tert-butyl dicarbonate (2.65 g) and nickel(II) chloride hexahydrate (147 mg), followed by the careful portion wise addition of sodium borohydride (1.60 g). The resulting black mixture was stirred for 15 min at 0-5° C. (ice bath), then the ice bath was removed and stirring was continued for 15 h at room temperature. Then diethylenetriamine (652 μL) was added and stirring at room temperature was continued for 15 min. The mixture was concentrated to dryness, ethyl acetate (60 mL) was added and the resulting suspension was washed with 1M aqueous ammonium chloride solution (60 mL), saturated aqueous sodium hydrogen carbonate (60 mL) and brine (60 mL), dried (MgSO₄), filtered and concentrated. The remaining residue was purified by flash chromatography (silica, cyclohexane/ethyl acetate) to afford the title compound (1.67 g; 74%). [(M-Boc)H]⁺=275, [MNa]⁺=397.

Step C

To a solution of the title compound from Step B above (1.61 g) in dry methanol (86 mL) was added palladium on charcoal (10 wt %, 458 mg). The resulting black mixture was degassed by three pump/vent with hydrogen cycles and then stirred at room temperature under a hydrogen atmosphere at normal pressure for 17 h. Filtration through a plug of Celite®, concentration and purification by flash chromatography (silica, cyclohexane/ethyl acetate) afforded the title compound (834 mg; 81%). [(M-Boc)H]⁺=141, [MNa]⁺=263.

Step D

To a solution of the title compound from Step C above (240 mg) in ethanol (4 mL) was added 3,4-diethoxy-3-cyclobutene-1,2-dione (261 mg). The resulting clear solution was heated to reflux for 14 h. and then concentrated. The remaining residue was purified by flash chromatography (silica, dichloromethane/methanol) to afford the title compound (294 mg; 81%). [(M-Boc)H]⁺=265, [MNa]⁺=387.

Step E

The title compound from Step D above (273 mg) was dissolved in a ˜7N solution of ammonia in methanol (10.7 mL). The reaction mixture was stirred at room temperature for 16 h and then concentrated to afford the title compound (246 mg; 98%). [(M-Boc)H]⁺=236, [MNa]⁺=358.

Step F

A 4M solution of hydrochloric acid in dioxane (2.8 mL) was added to a suspension of the crude title compound from Step E above (235 mg) in dry methanol (2.8 mL). The reaction mixture was stirred at room temperature for 1 h and then concentrated to afford the title compound (189 mg; 99%). [MH]⁺=236.

### Preparative Example 2025

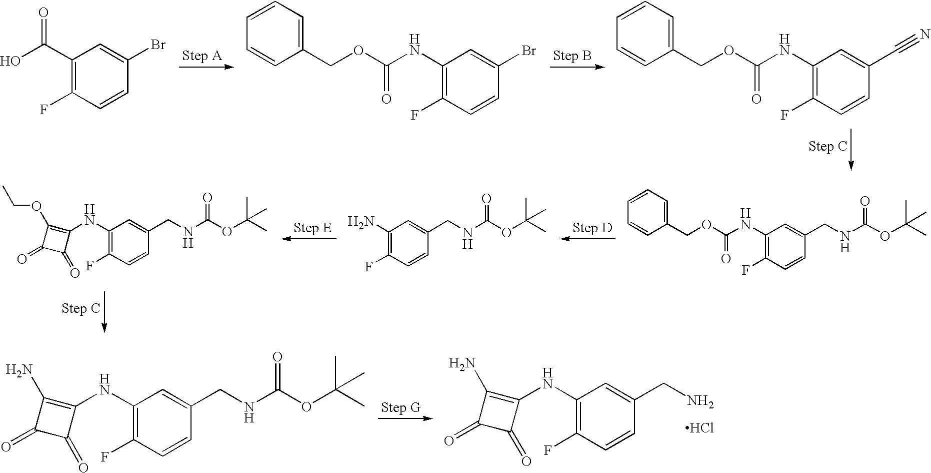 Step A

To a suspension of commercially available 5-bromo-2-fluoro-benzoic acid (4.52 g) in dry toluene (200 mL) were added triethylamine (3.37 mL) and diphenylphosphoryl azide (5.28 mL). The resulting clear solution was heated to reflux for 16V₂ h. Then benzyl alcohol (2.51 mL) was added and heating to reflux was continued for 3 h. The mixture was concentrated and purified by flash chromatography (silica, cyclohexane/ethyl acetate) to afford the title compound (2.96 g; 46%). [MH]⁺=324/326, [MNa]⁺=346/348.

Step B

The title compound from Step B above (1.62 g), zinc(II) cyanide (479 mg) and tetrakis triphenylphosphine palladium(0) (292 mg) were suspended in dry N,N-dimethylformamide (10 mL). The resulting mixture was degassed by three pump/vent cycles with argon and then placed in a preheated oil bath (˜80° C.). After stirring at this temperature for 20 h the mixture was cooled to room temperature, diluted with water (100 mL) and extracted with ethyl acetate (3×100 mL). The combined organic layers were washed with water (2×100 mL) and saturated aqueous sodium chloride (100 mL), dried (MgSO₄), filtered and concentrated. The remaining residue was purified by flash chromatography (silica, cyclohexane/ethyl acetate) to afford the title compound (761 mg; 56%). [MH]⁺=271.

Step C

To an ice cooled (0-5° C.) solution of the title compound from Step B above (761 mg) in dry methanol (28 mL) were added di-tert-butyl dicarbonate (1.27 g) and nickel(II) chloride hexahydrate (69 mg), followed by the careful portion wise addition of sodium borohydride (752 mg). The resulting black mixture was stirred for 20 min at 0-5° C. (ice bath), then the ice bath was removed and stirring was continued for 16½ h at room temperature. Then diethylenetriamine (302 μL) was added and stirring at room temperature was continued for 30 min. The mixture was concentrated to dryness, ethyl acetate (28 mL) was added and the resulting suspension was washed with 1M aqueous ammonium chloride (28 mL), saturated aqueous sodium hydrogen carbonate (28 mL) and brine (28 mL), dried (MgSO₄), filtered and concentrated to afford the analytically pure title compound (943 mg; 89%). [(M-Boc)H]⁺=275, [MNa]⁺=397.

Step D

To a solution of the title compound from Step C above (898 mg) in dry methanol (48 mL) was added palladium on charcoal (10 wt %, 255 mg). The resulting black mixture was degassed by three pump/vent cycles with hydrogen and then stirred at room temperature under a hydrogen atmosphere at normal pressure for 16½ h. Filtration through a plug of Celite® and concentration the analytically pure title compound (554 mg; 96%). [(M-Boc)H]⁺=141, [MNa]⁺=263.

Step E

To a solution of the title compound from Step D above (240 mg) in ethanol (4 mL) was added 3,4-diethoxy-3-cyclobutene-1,2-dione (261 mg). The resulting clear solution was heated to reflux for 24 h and then concentrated. The remaining residue was purified by flash chromatography (silica, dichloromethane/methanol) to afford the title compound (259 mg; 71%). [(M-Boc)H]⁺=265, [MNa]⁺=387.

Step F

The title compound from Step E above (226 mg) was dissolved in a ˜7N solution of ammonia in methanol (8.7 mL). The reaction mixture was stirred at room temperature for 16 h and then concentrated to afford the title compound (204 mg; 98%). [(M-Boc)H]⁺=236, [MNa]⁺=358.

Step G

A 4M solution of hydrochloric acid in dioxane (2.4 mL) was added to a suspension of the crude title compound from Step E above (205 mg) in dry methanol (2.4 mL). The reaction mixture was stirred at room temperature for 1 h and then concentrated to afford the title compound (164 mg; 99%). [M-Cl]⁺=236.

### Preparative Example 2026

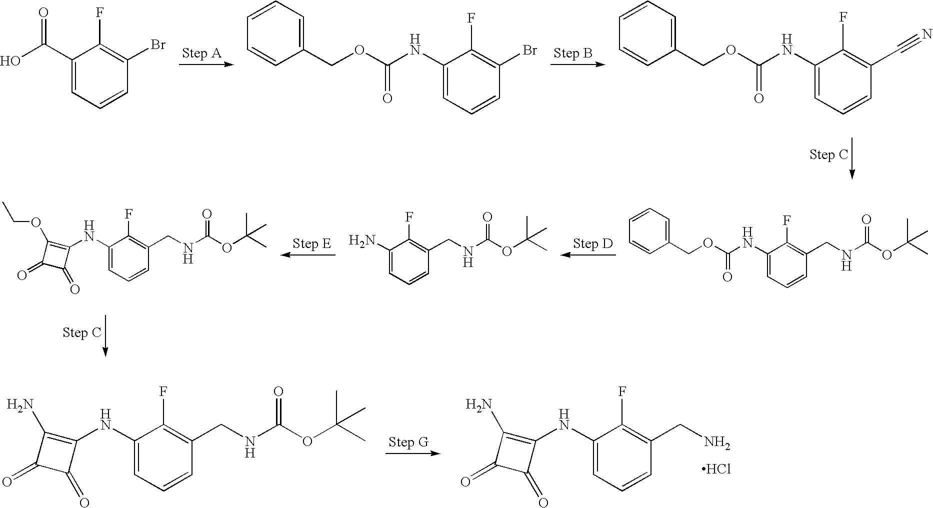 Step A

To a suspension of commercially available 3-bromo-2-fluoro-benzoic acid (876 mg) in dry toluene (40 mL) were added triethylamine (675 μL) and diphenylphosphoryl azide (1.06 mL). The resulting clear solution was heated to reflux for 16½ h. Then benzyl alcohol (502 μL) was added and heating to reflux was continued for 3 h. The mixture was concentrated and purified by flash chromatography (silica, cyclohexane/ethyl acetate) to afford the title compound (596 mg; 46%). [MH]⁺=324/326, [MNa]⁺=346/348.

Step B

The title compound from Step B above (536 mg), zinc(II) cyanide (151 mg) and tetrakis triphenylphosphine palladium(0) (92 mg) were suspended in dry N,N-dimethylformamide (3.1 mL). The resulting mixture was degassed by three pump/vent cycles with argon and then placed in a preheated oil bath (˜80° C.). After stirring at this temperature for 19½ h the mixture was cooled to room temperature, diluted with water (31 mL) and extracted with ethyl acetate (3×31 mL). The combined organic layers were washed with water (2×31 mL) and brine (31 mL), dried (MgSO₄), filtered and concentrated. The remaining residue was purified by flash chromatography (silica, cyclohexane/ethyl acetate) to afford the title compound (234 mg; 55%). [MH]⁺=271.

Step C

To an ice cooled (0-5° C.) solution of the title compound from Step B above (234 mg) in dry methanol (9 mL) were added di-tert-butyl dicarbonate (390 mg) and nickel(II) chloride hexahydrate (21 mg), followed by the careful portion wise addition of sodium borohydride (229 mg). The resulting black mixture was stirred for 20 min at 0-5° C. (ice bath), then the ice bath was removed and stirring was continued for 14 h at room temperature. Then diethylenetriamine (95 μL) was added and stirring at room temperature was continued for 1 h. The mixture was concentrated to dryness, ethyl acetate (9 mL) was added and the resulting suspension was washed with 1M aqueous ammonium chloride (9 mL), saturated aqueous sodium hydrogen carbonate (9 mL) and brine (9 mL), dried (MgSO₄), filtered and concentrated to afford the analytically pure title compound (266 mg; 82%). [(M-Boc)H]⁺=275, [MNa]⁺=397.

Step D

To a solution of the title compound from Step C above (266 mg) in dry methanol (14 mL) was added palladium on charcoal (10 wt %, 76 mg). The resulting black mixture was degassed by three pump/vent cycles with hydrogen and then stirred at room temperature under a hydrogen atmosphere at normal pressure for 13½ h. Filtration through a plug of Celite®, concentration and purification by flash chromatography (silica, cyclohexane/ethyl acetate) afforded the title compound (121 mg; 71%). [(M-isobutene)H]⁺=184, [MNa]⁺=263.

Step E

To a solution of the title compound from Step D above (110 mg) in ethanol (1.8 mL) was added 3,4-diethoxy-3-cyclobutene-1,2-dione (119 mg). The resulting clear solution was heated to reflux for 17½ h. and then concentrated. The remaining residue was purified by flash chromatography (silica, dichloromethane/methanol) to afford the title compound (90 mg; 54%). [(M-Boc)H]⁺=265, [MNa]⁺=387.

Step F

The title compound from Step E above (80 mg) was dissolved in a ˜7N solution of ammonia in methanol (3.1 mL). The reaction mixture was stirred at room temperature for 2½ h and then concentrated to afford the title compound (73 mg; 99%). [(M-Boc)H]⁺=236, [MNa]⁺=358.

Step G

A 4M solution of hydrochloric acid in dioxane (775 μL) was added to a suspension of the crude title compound from Step F above (65 mg) in dry methanol (775 mL). The reaction mixture was stirred at room temperature for 3 h and then concentrated to afford the title compound (52 mg; 99%). [M-Cl]⁺=236.

### Preparative Example 2027

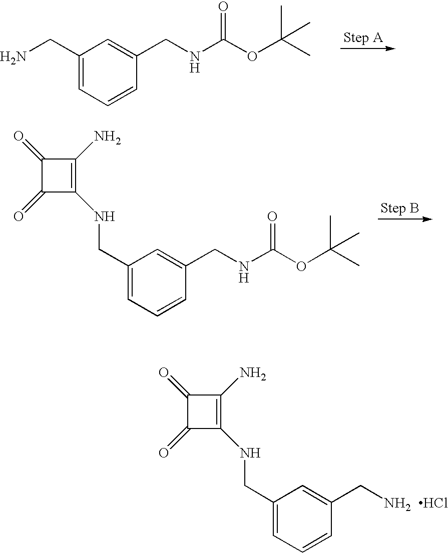 Step A

To a solution of 3,4-diethoxy-3-cyclobutene-1,2-dione (1.3 mL) in ethanol (40 mL) was added commercially available 1-(N-Boc-aminomethyl)-3-(aminomethyl)benzene (1.39 g). After 2 h ammonia (28% aqueous solution, 40 mL) was added and the mixture was stirred for additional 2 h and then evaporated under reduced pressure. The residue was slurried in methanol (20 mL) and filtered to give the intermediate (1.6 g; 82%).

Step B

A solution of the intermediate from step A above (400 mg) in hydrogen chloride (4M solution in dioxane) was stirred for 14 h, evaporated and dried to afford the title compound (317 mg; 98%) as an off-white solid. [M-Cl]⁺=232.

### Preparative Example 2028

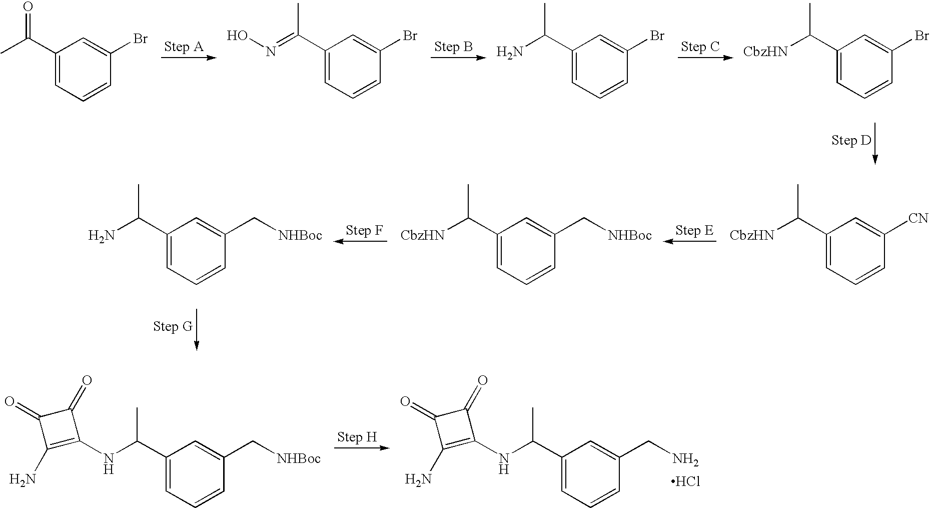 Step A

Commercially available 3-bromoacetophenone (4 g) was dissolved in methanol (50 mL). Hydroxylamine hydrochloride (6.9 g) and sodium hydrogencarbonate (8.4 g) were added and the mixture was refluxed for 1.5 h. After cooling to room temperature the mixture was diluted in water and extracted with ethyl acetate. The organic layer was dried (MgSO₄) and concentrated to afford the intermediate (4.2 g; 98%) as a colourless solid; [MH]⁺=214/216.

Step B

The intermediate from step A above (4.2 g) was dissolved in methanol (150 mL). 6N hydrochloric acid (150 mL) and zinc dust were added in small porions and the mixture was refluxed for 3 h. After cooling to room temperature sodium hydroxide was added and the precipitate was filtered off and the filtrate concentrated under reduced pressure. The residue was then redissolved in water and extracted with ethyl acetate. The organic layer was dried (MgSO₄) and concentrated to afford the intermediate (3 g; 77%) as a colourless solid. [MH]⁺=200/202.

Step C

The intermediate from step B above (3 g) was dissolved in water/THF 1:1 (150 mL). Potassium carbonate (2.5 g) and benzyl chloroformate (4.6 mL) were added and the mixture was stirred at room temperature overnight. The reaction mixture was extracted with ethyl acetate. The organic layer was dried (MgSO₄), concentrated and purified by column chromatography (silica, dichloromethane) to afford the intermediate (3 g; 60%) as a colourless solid. [MH]⁺=334/336.

Step D

The intermediate from Step C above (3 g), zinc(II) cyanide (800 mg) and tetrakistriphenylphosphine palladium(0) (520 mg) were suspended in dry N,N-dimethylformamide (40 mL). The resulting mixture was degassed by three pump/vent cycles with argon and then placed in a preheated oil bath (˜80° C.). After stirring at this temperature for 20 h, the mixture was cooled to room temperature, diluted with water (100 mL) and extracted with ethyl acetate (3×100 mL). The combined organic layers were washed with water (2×100 mL) and brine (100 mL), dried (MgSO₄), filtered and concentrated. The remaining residue was purified by chromatography (silica, dichloromethane) to afford the title compound (1.3 g; 52%). [MH]⁺=281.

Step E

To an ice cooled solution of the title compound from Step D above (1.3 g) in dry methanol (40 mL) were added di-tert-butyl dicarbonate (2 g) and nickel(II) chloride hexahydrate (120 mg), followed by the careful portion wise addition of sodium borohydride (1.2 g). The resulting black mixture was stirred for 20 min at 0-5° C. (ice bath), then the ice bath was removed and stirring was continued overnight at room temperature. Then diethylenetriamine (1 mL) was added and stirring at room temperature was continued for 30 min. The mixture was concentrated to dryness, ethyl acetate was added and the resulting suspension was washed with 1M aqueous ammonium chloride solution, saturated aqueous sodium hydrogencarbonate and brine, dried (MgSO₄), filtered and concentrated to afford the analytically pure title compound (1.3 mg; 56%). [MH]⁺=384.

Step F

To a solution of the title compound from Step E above (1.3 g) in dry methanol (40 mL) was added palladium on charcoal (10 wt %, 140 mg). The resulting black mixture was degassed by three pump/vent cycles with hydrogen and then stirred at room temperature under a hydrogen atmosphere at normal pressure overnight. Filtration through a plug of Celite® and concentration results in the analytically pure title compound (950 mg; 96%). [MH]⁺=251.

Step G

To a solution of the title compound from Step F above (950 mg) in ethanol (4 mL) was added triethylamine (0.7 mL) and 3,4-diethoxy-3-cyclobutene-1,2-dione (782 mg). The resulting clear solution was heated to reflux overnight. After cooling to room temperature, aqueous ammonia (30% aqueous solution, 30 mL) was added and the mixture was stirred for another 2 h at room temperature and concentrated to afford the title compound (1.3 g; 91%). [(M-Boc)H]⁺=275.

Step H

A 4M solution of hydrochloric acid in dioxane (5 mL) was added to a suspension of the title compound from Step G above (1.3 g) in dioxane (5 mL). The reaction mixture was stirred at room temperature overnight and was then concentrated to afford the title compound (950 mg; 99%). [M-Cl]⁺=246.

### Preparative Example 2029

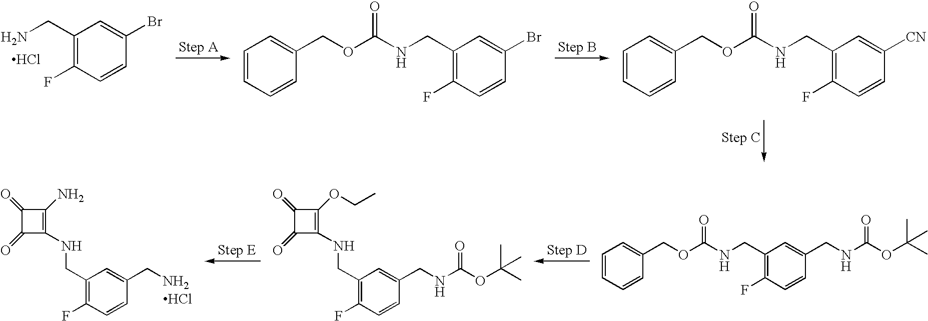 Step A

A solution of 5-bromo-2-fluorobenzylamine hydrochloride (5.39 g), potassium carbonate (7.74 g) and benzyl chloroformate (3.8 mL) in THF/water was stirred for 90 min and evaporated under reduced pressure. The residue was diluted with ethyl acetate, washed with 10% citric acid, saturated sodium hydrogen carbonate and brine, dried (MgSO₄), concentrated and slurried in pentane. Filtration afforded the intermediate (7.74 g; quantitative) as colourless needles. [MH]⁺=338/340.

Step B

The intermediate from step A above (7.74 g), zinc(II) cyanide (2.0 g) and tetrakis(triphenylphosphine)palladium(0) (1.32 g) were dissolved in dry DMF (30 mL), degassed and stirred at 85° C. under argon. After 16 h the mixture was evaporated and diluted with ethyl acetate. The solution was washed with saturated ammonium chloride and brine, dried (MgSO₄), concentrated and purified by column chromatography (silica, cyclohexane/EtOAc, 9:1 to 7:3) to afford the intermediate (6.25 g; 98%) as colourless crystals. ¹H-NMR (CDCl₃) δ=4.42 (d, 2 H), 5.13 (s, 2 H), 5.22 (br s, 1 H), 7.1-7.75 (m, 8 H).

Step C

The intermediate from step B above (3.25 g), di-tert-butyl dicarbonate (5.0 g) and nickel(II) chloride hexahydrate (300 mg) was dissolved in methanol (100 mL) and cooled to 0° C. Then sodium borohydride (2.6 g) was added in portions and the ice bath was removed. The mixture was vigorously stirred for 1 h, then diethylenetriamine (2 mL) was added and the mixture was concentrated to dryness. The residue was diluted with ethyl acetate, washed with 10% citric acid, saturated sodium hydrogen carbonate and brine, dried (MgSO₄), concentrated and purified by column chromatography (silica, cyclohexane/EtOAc, 7:3 to 6:4) to afford the intermediate (4.09 g; quantitative) as colourless oil.

Step D

To a solution of intermediate from step C above (4.09 g) in ethanol (100 mL) was added palladium on charcoal (10 wt %, 600 mg) and then hydrogenated unter normal pressure overnight. The catalyst was filtered off and the solvent was evaporated to 20 mL. Then 3,4-diethoxy-3-cyclobutene-1,2-dione (2.22 mL) and trietylamine (1 mL) was added and the mixture was refluxed for 9 h. The resulting solution was divided in two portions and used in the next steps without further purification. [(M-Boc)H]⁺=279, [MNa]⁺=401.

Step E

To one portion of intermediate from step D above was added ammonia (28% aqueous solution, 60 mL) and the mixture was stirred for additional 2 h and then evaporated under reduced pressure. The precipitate was filtered and washed with water and then tetrahydrofurane and dried in vaccuo. The remaining solid was suspended in hydrogen chloride (4M solution in dioxane, 15 mL) and stirred overnight, evaporated, suspended in tetrahydrofurane, filtered and dried to afford the title compound (1.03 g; 34% inc. Step D) as an off-white solid. [M-Cl]⁺=250.

### Preparative Example 2030

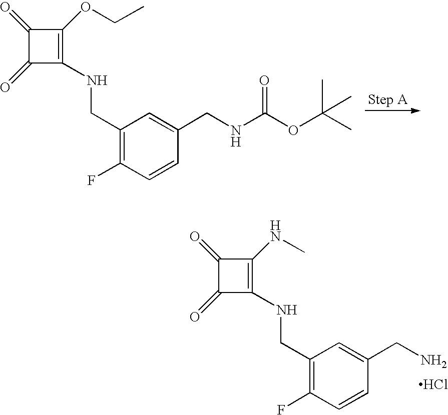 Step A

To one portion of intermediate from the Preparative Example 2029, step D above was added methylamine (40% aqueous solution, 60 mL) and the mixture was stirred overnight and then evaporated under reduced pressure. The remaining solid was absorbed on silica and purified by column chromatography (dichloromethane/methanol, 95:5 to 9:1). The remaining solid was dissolved in hydrogen chloride (4M solution in dioxane, 20 mL) and stirred for 3 h and evaporated to afford the title compound (414 mg) as an off-white solid. [M-Cl]⁺=264.

### Preparative Example 2031

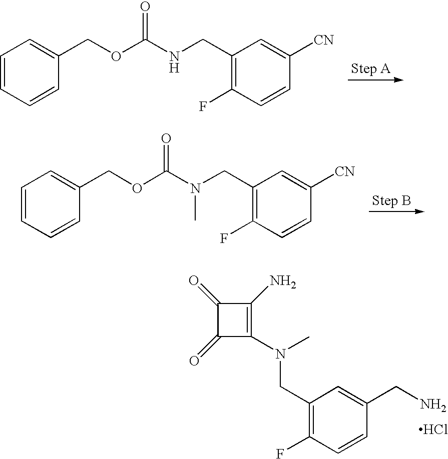 Step A

The intermediate from the Preparative Example 2029, Step B above (1.1 g) was dissolved in N,N-dimethylformamide (20 mL) and the mixture was cooled to 0° C. After adding sodium hydride (102 mg) and iodomethane (0.5 mL), the reaction mixture was allowed to warm up to room temperature and stirred overnight. The solvent was removed and the resulting residue was redissolved in water and extracted with ethyl acetate. The organic layer was dried (MgSO₄) and concentrated to afford the intermediate (1.02 g). [MH]⁺=299.

Step B

The intermediate from Step A above (1.02 g) was treated as described in the Preparative Example 2029, step C to step E, to afford the title compound (646 mg; 50%) as an off-white solid. [M-Cl]⁺=264.

### Preparative Example 2032

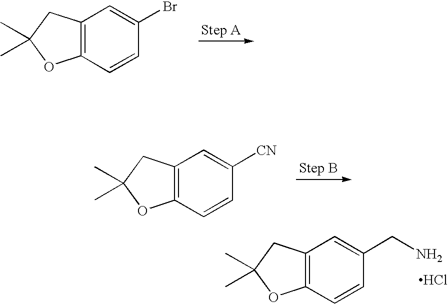 Step A

A suspension of 5-bromo-2,2-dimethyl-2,3-dihydro-benzofuran (A. M. Bernard et al., Synthesis, 1997, 41-43) (2.32 g) and copper(I) cyanide (1.35 g) in N-methylpyrrolidone was heated in a sealed tube to 160° C. for 3 days. After evaporation of the solvent the residue was purified by column chromatography (silica, cyclohexane/EtOAc, 1:0 to 10: 1) to afford the intermediate (1.26 g; 71%) as a colourless oil.

Step B

The intermediate from Step A above (1.26 g), di-tert-butyl dicarbonate (3.2 g) and nickel(II) chloride hexahydrate (180 mg) was dissolved in dry methanol (30 mL) and cooled to 0° C. Then sodium borohydride (2 g) was added in portions and the ice bath removed. The mixture was vigorously stirred overnight, then diethylenetriamine (500 μL) was added and the mixture was concentrated to dryness. The residue was diluted with ethyl acetate, washed with 10% citric acid, saturated sodium hydrogen carbonate and brine, dried (MgSO₄) and concentrated. Purification by column chromatography (silica, cyclohexane/EtOAc, 9:1) afforded a clear oil, which was dissolved in hydrogen chloride (4M solution in dioxane, 20 mL) and stirred overnight, filtered and washed with diethyl ether to afford the title compound (881 mg; 57%) as colourless fluffy crystals. [M-NH₃Cl]⁺=161.

### Preparative Example 2033

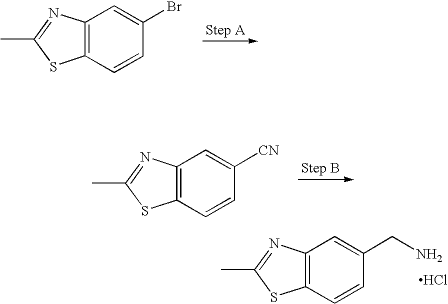 Step A

Commercially available 5-bromo-2-methylbenzothiazole (1.42 g), zinc(II) cyanide (584 mg) and tetrakis(triphenylphosphine)palladium(0) (360 mg) were dissolved in dry DMF (12 mL), degassed and stirred at 80° C. under argon. After 16 h the mixture was evaporated and diluted with chloroform. The solution was washed with 1N hydrochloric acid, 1N sodium hydroxide and brine, dried (MgSO₄) and absorbed on silica. Purification by column chromatography (cyclohexane/EtOAc, 8:2 to 7:3) afforded the intermediate (1.01 g; 93%) as bright yellow needles.

Step B

The intermediate from Step A above (1.01 g), di-tert-butyl dicarbonate (2.54 g) and nickel(II) chloride hexahydrate (140 mg) were dissolved in dry methanol (60 mL) and cooled to 0° C. Then sodium borohydride (1.6 g) was added in portions and the ice bath was removed. The mixture was vigorously stirred for 5 h, then diethylenetriamine (500 μL) was added and the mixture was concentrated to dryness. The residue was diluted with ethyl acetate, washed with 10% citric acid, saturated sodium hydrogen carbonate and brine, dried (MgSO₄) and concentrated. Purification by column chromatography (silica, cyclohexane/EtOAc, 8:2 to 6:4) afforded a yellow oil, which was suspended in hydrogen chloride (4M solution in dioxane, 20 mL) and stirred overnight, filtered and washed with diethyl ether to afford the title compound (455 mg; 37%) as a colourless solid. [M-NH₃Cl]⁺=162; [M-Cl]⁺=179.

### Preparative Example 2034

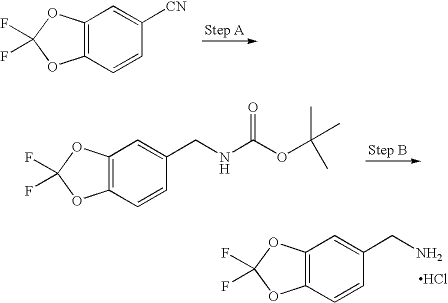 Step A

Commercially available 2,2-difluoro-benzo[1,3]dioxole-5-carbonitrile (1.34 g), di-tert-butyl dicarbonate (3.2 g) and nickel(II) chloride hexahydrate (174 mg) was dissolved in dry methanol (40 mL) and cooled to 0° C. Then sodium borohydride (1.9 g) was added in portions and the ice bath removed. The mixture was vigorously stirred for 2 h, then diethylenetriamine (500 μL) was added and the mixture was concentrated to dryness. The residue was diluted with ethyl acetate, washed with 10% citric acid, saturated sodium hydrogen carbonate and brine, dried (MgSO₄), concentrated and purified by column chromatography (silica, cyclohexane/EtOAc, 95:5 to 8:2) to afford the intermediate (1.44 g; 68%) as colourless oil, which crystallized upon standing.

Step B

A solution of the intermediate from Step A above (1.44 g) in hydrogen chloride (4M solution in dioxane, 30 mL) was stirred overnight, diluted with diethyl ether and the colourless precipitate was filtered and washed with diethyl ether to afford the title compound (1.01 g; 90%) as fluffy colourless crystals. [M-NH₃Cl]⁺=171; [M-Cl]⁺=188.

### Preparative Example 2035

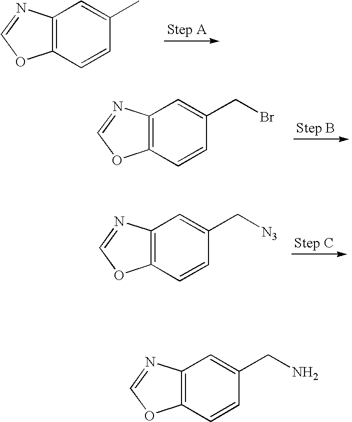 Step A

A mixture of commercially available 5-methyl-benzooxazole (2.00 g), N-bromosuccinimide (3.48 g) and α,α′-azoisobutyronitrile (49 mg) in chloroform (40 mL) was refluxed for 2 h. The mixture was concentrated and purified by column chromatography (silica, cyclohexane/EtOAc, 9:1) to afford the title compound (1.92 g; 61%) as a solid. [MH]⁺=212.

Step B

A mixture of the title compound from Step A above (869 mg) and sodium azide (1.33 g) in DMF (20 mL) was stirred at 60° C. for 16 h. The mixture was concentrated and the residue dissolved in ethyl acetate. The organic layer was washed with water and saturated sodium hydrogen carbonate, dried (MgSO₄) and concentrated to afford the title compound (648 mg; 91%) as an oil. [MH]⁺=175.

Step C

A solution of the title compound from Step B above (91 mg) and triphenylphosphine (178 mg) in tetrahydrofurane (2.5 mL) was stirred at room temperature for 3 h. Then water (1 mL) was added and the mixture was stirred for 16 h at room temperature. The mixture was concentrated and purified by column chromatography (silica, chloroform/MeOH, 85:15) to afford the title compound (35 mg; 45%) as a glass. [MH]⁺=149.

### Preparative Example 2036

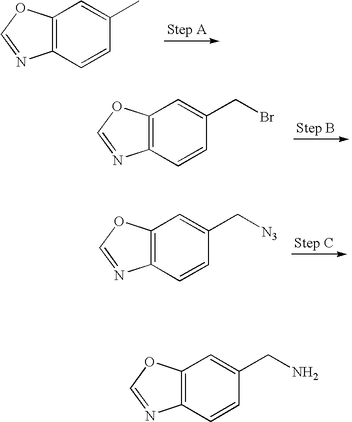 Step A

A mixture of commercially available 6-methyl-benzooxazole (1.00 g), N-bromosuccinimide (1.74 g) and α,α′-azoisobutyronitrile (25 mg) in chloroform (20 mL) was refluxed for 2 h. The mixture was concentrated and purified by column chromatography (silica, cyclohexane/EtOAc, 9:1) to afford the title compound (550 mg; 30%) as a solid. [MH]⁺=212.

Step B

A mixture of the title compound from Step A above (473 mg) and sodium azide (725 mg) in DMF (10 mL) was stirred at 60° C. for 16 h. The mixture was concentrated and the residue dissolved in ethyl acetate. The organic layer was washed with water and saturated sodium hydrogen carbonate, dried (MgSO₄) and concentrated to afford the title compound as an oil. [MH]⁺=175.

Step C

A solution of the title compound from Step B above (101 mg) and triphenylphosphine (198 mg) in tetrahydrofurane (2.5 mL) was stirred at room temperature for 16 h. Then water (1 mL) was added and the mixture was stirred for 4 h at room temperature. The mixture was concentrated and purified by column chromatography (silica, chloroform/MeOH, 85:15) to afford the title compound (62 mg; 72%) as a glass. [MH]⁺=149.

### Preparative Example 2037

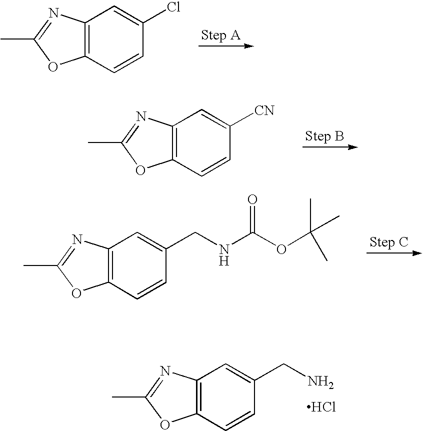 Step A

Commercially available 5-chloro-2-methylbenzoxazole (1.5 g), potassium cyanide (612 mg), dipiperidinomethane (720 μL), palladium diacetate (80 mg) and 1,5-bis-(diphenylphosphino)pentane (315 mg) were dissolved in dry toluene (20 mL), degassed and stirred at 160° C. in a sealed pressure tube under argon. After 24 h the mixture was diluted with ethyl acetate. The organic layer was washed with saturated ammonium chloride and brine, dried (MgSO₄), concentrated and purified by column chromatography (silica, cyclohexane/EtOAc, 9:1 to 7:3) to afford the intermediate (372 mg; 26%) as a colourless solid. ¹H-NMR (CDCl₃) δ=2.63 (s, 3 H), 7.48-7.58 (s, 2 H), 7.90 (s, 1 H).

Step B

The intermediate from step A above (372 mg), di-tert-butyl dicarbonate (1.02 g) and nickel(II) chloride hexahydrate (56 mg) were dissolved in dry methanol (25 mL) and cooled to 0° C. Then sodium borohydride (400 mg) was added in portions and the ice bath removed. The mixture was vigorously stirred for 14 h, then diethylenetriamine (300 μL) was added and the mixture was concentrated to dryness. The residue was diluted with ethyl acetate, washed with 10% citric acid, saturated sodium hydrogen carbonate and brine, dried (MgSO₄), concentrated and purified by column chromatography (silica, cyclohexane/EtOAc, 7:3 to 6:4) to afford the intermediate (413 mg) as a colourless oil.

Step C

A solution of the intermediate from step B above (413 mg) in hydrogen chloride (4M solution in dioxane) was stirred for 2 h, diluted with diethyl ether and the precipitate was filtered, washed with diethyl ether to afford the title compound (341 mg; 73% over two steps) as a colourless solid. [M-Cl]=163.

### Preparative Example 2038

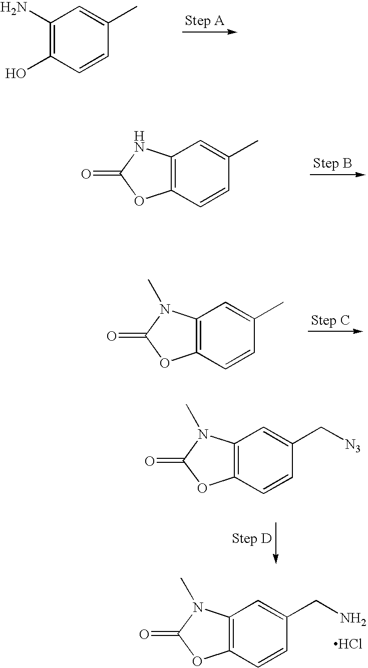 Step A

Commercially available 2-hydroxy-5-methylaniline (5.2 g) and N,N′-carbonyldiimidazole (6.85 g) were refluxed in dry THF (60 mL) for 6 h, cooled to room temperature, poured on ice and adjusted to pH 4 with 6N hydrochloric acid. The precipitate was filtered off, dried and recrystallized from toluene to afford the intermediate (4.09 g; 65%) as a grey solid.

Step B

The intermediate from step A above (1.5 g), potassium carbonate (1.7 g) and methyl iodide (6 mL) were dissolved in dry DMF (15 mL) and stirred at 50C for 2 h. The mixture was concentrated to dryness and acidified to pH 4 with 1N hydrochloric acid. The precipitate was filtered off and dried to afford the intermediate (1.48 g; 90%) as an off-white solid. ¹H-NMR (CDCl₃) δ=2.40 (s, 3 H), 3.38 (s, 3 H), 6.77 (s, 1 H), 6.90 (d, 1 H), 7.05 (s, 1 H).

Step C

The intermediate from step B above (1.1 g), N-bromosuccinimide (1.45 g) and α,α′-azoisobutyronitrile (150 mg) were suspended in carbon tetrachloride (50 mL), degassed with argon and heated to reflux. After 1 h the mixture was cooled, filtered, evaporated and dissolved in dry DMF (20 mL). Then sodium azide (1 g) was added and the mixture was vigorously stirred for 3 h, diluted with ethyl acetate, washed with water and brine, dried (MgSO₄), concentrated and purified by column chromatography (silica, cyclohexane/EtOAc, 8:2 to 7:3) to afford the intermediate (963 mg; 70%) as colourless needles. ¹H-NMR (CDCl₃) δ=3.36 (s, 3 H), 4.25 (s, 2 H), 6.88 (s, 1 H), 6.98 (d, 1 H), 7.07 (s, 1 H).

Step D

The intermediate from step C above (963 mg) and triphenylphosphine (1.36 g) in THF (30 mL) were stirred for 14 h, then water was added and the mixture was stirred for additional 2 h. The mixture was evaporated, coevaporated twice with toluene and diluted with dry dioxane. After addition of hydrogen chloride (4M solution in dioxane, 1.5 mL), the precipitate was filtered off and dried to afford the intermediate (529 mg; 52%) as a colourless solid. [M-Cl]⁺=179.

### Preparative Example 2039

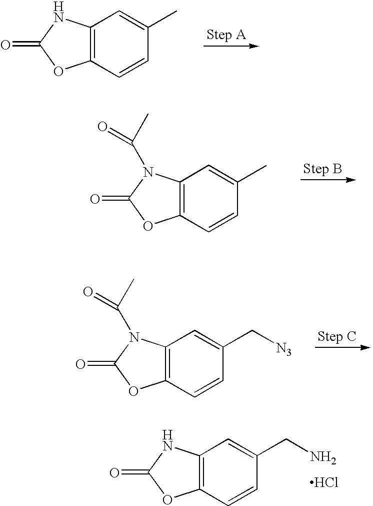 Step A

5-Methyl-3H-benzooxazol-2-one (1.58 g) was heated in acetic acid anhydride (20 mL) to 80° C. for 2 h, evaporated and coevaporated with toluene to afford the intermediate (2.2 g; quantitative) as a colourless solid. [MH]⁺=192.

Step B

The intermediate from step A above was treated as described in Preparative Example 2038, Step C.

Step C

To the intermediate from step B above (45 mg) was deacetylated in methanol (10 mL) by adding 2N sodium carbonate (10 mL) and heating to 60° C. for 30 min. The resulting intermediate was treated as described in Preparative Example 2038, Step D. [M-Cl]⁺=165.

### Preparative Example 2040

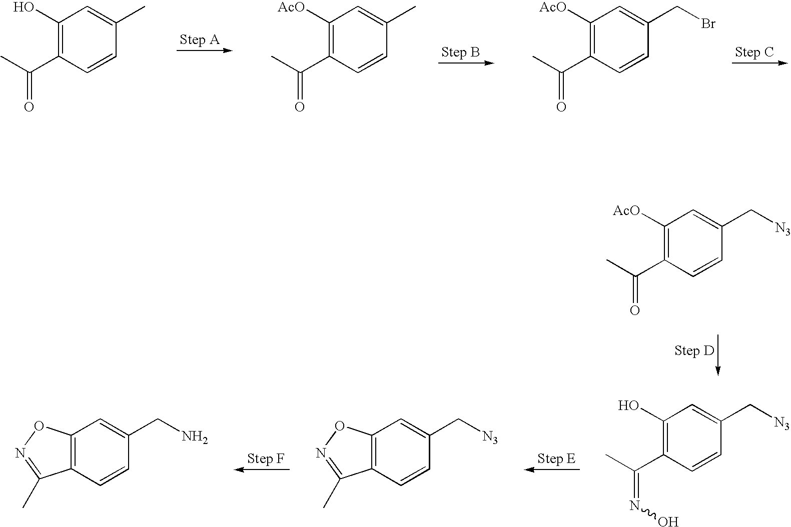 Step A

A solution of commercially available 1-(2-hydroxy-4-methyl-phenyl)-ethanone (5.00 g) and acetic acid anhydride (4.08 g) in pyridine was stirred for 18 h at room temperature. The mixture was concentrated and the residue was dissolved in ethyl acetate. The organic layer was washed with saturated sodium hydrogen carbonate, saturated ammonium hydrochloride and brine, dried (MgSO₄) and concentrated to afford the title compound (6.04 g; 95%) as an oil. [MH]⁺=193.

Step B

A mixture of the title compound from step A above (3.54 g), N-bromosuccinimide (4.27 g) and α,α′-azoisobutyronitrile (151 mg) in tetrachloromethane (30 mL) was refluxed for 6 h. After the precipitate was filtered off, the organic layer was concentrated and purified by column chromatography (silica, cyclohexane/EtOAc, 8:2) to afford the title compound (1.70 g; 34%) as an oil. [MH]⁺=271/273.

Step C

A mixture of the title compound from step B above (553 mg) and sodium azide (398 mg) in DMF (8 mL) was stirred at room temperature for 1.5 h. The mixture was concentrated and the residue was dissolved in ethyl acetate. The organic layer was washed with water and saturated sodium hydrogen carbonate, dried (MgSO₄), concentrated and purified by column chromatography (silica, cyclohexane/EtOAc, 8:2) to afford the title compound (213 mg; 44%) as an oil. [MH]⁺=234.

Step D

A mixture of the title compound of step C above (213 mg), hydroxylamine hydrochloride (507 mg) and sodium hydrogen carbonate (614 mg) in methanol (4 .mL) was stirred at 60° C. for 16 h. The mixture was diluted with ethyl acetate, washed with 0.01M hydrochloric acid, dried (MgSO₄) and concentrated to afford the title compound (165 mg; 88%) as a colourless solid. [MH]⁺=207.

Step E

To a solution of the title compound from step D above (156 mg) and pyridine (597 mg) in diethyl ether (3 mL) was added thionyl chloride (90 mg) at 0° C. and the mixture was stirred at room temperature for 16 h. The mixture was diluted with 0.01M hydrochloric acid, extracted with ethyl acetate, dried (MgSO₄) and concentrated to afford the title compound (110 mg; 77%) as a colourless solid. [MH]⁺=189.

Step F

A solution of the title compound from step E above (105 mg) and triphenylphosphine (191 mg) in tetrahydrofurane (2.5 mL) was stirred at room temperature for 16 h. Then water (1 mL) was added and the mixture was stirred for 4 h at room temperature. The mixture was concentrated and purified by column chromatography (silica, chloroform/MeOH, 85:15) to afford the title compound (49 mg; 54%) as an oil. ¹H-NMR (CDCl₃) δ=2.52 (s, 3 H), 3.85 (s, 2 H), 7.18 (d, 1 H), 7.40 (s, 1 H), 7.50 (d, 1 H).

### Preparative Example 2041

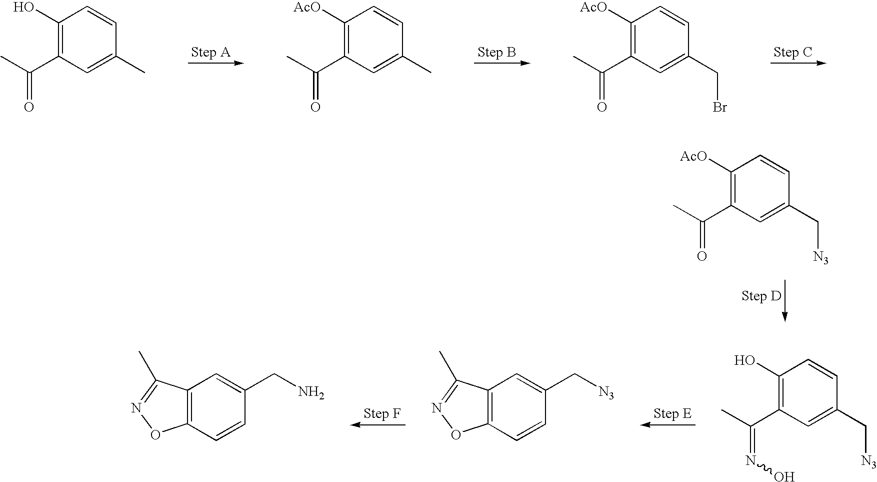 Step A

A solution of commercially available 1-(2-hydroxy-5-methyl-phenyl)-ethanone (5.00 g) and acetic acid anhydride (4.08 g) in pyridine was stirred for 16 h at room temperature. The mixture was concentrated and the residue dissolved in ethyl acetate. The organic layer was washed with saturated sodium hydrogen carbonate, saturated ammonium hydrochloride and brine, dried (MgSO₄) and concentrated to afford the title compound (5.97 g; 95%) as an oil which crystallized upon standing. [MH]⁺=193.

Step B

A mixture of the title compound from step A above (5.97 g), N-bromosuccinimide (8.30 g) and α,α′-azoisobutyronitrile (102 mg) in tetrachloromethane (60 mL) was refluxed for 4 h. After the precipitate was filtered off, the organic layer was concentrated and purified by column chromatography (silica, cyclohexane/EtOAc, 8:2) to afford the title compound (3.16 g; 37%) as a colourless solid. [MH]⁺=271/273.

Step C

A mixture of the title compound from step B above (3.16 g) and sodium azide (398 mg) in DMF (50 mL) was stirred at room temperature for 1.5 h. The mixture was concentrated and the residue was dissolved in ethyl acetate. The organic layer was washed with water and saturated sodium hydrogen carbonate, dried (MgSO₄), concentrated and purified by column chromatography (silica, cyclohexane/EtOAc, 8:2) to afford the title compound (639 mg; 23%) as an oil. [MH]⁺=234.

Step D

A mixture of the title compound of step C above (630 mg), hydroxylamine hydrochloride (1.50 g) and sodium hydrogen carbonate (1.82 g) in methanol (4 mL) was stirred at 60° C. for 16 h. The mixture was diluted with ethyl acetate, washed with 0.O1M hydrochloric acid, dried (MgSO₄), concentrated and purified by column chromatography (silica, cyclohexane/EtOAc, 8:2) to afford the title compound (80 mg; 14%) as a colourless solid. [MH]⁺=207.

Step E

To a solution of the title compound from step D above (80 mg) and pyridine (285 mg) in diethyl ether (3 mL) was added thionyl chloride (34 mg) at 0° C. and the mixture was stirred at room temperature for 16 h. The mixture was diluted with 0.01M hydrochloric acid, extracted with ethyl acetate, dried (MgSO₄) and concentrated to afford the title compound (50.1 mg; 74%) as an oil. [MH]⁺=189.

Step F

A solution of the title compound from step E above (50 mg) and triphenylphosphine (91 mg) in tetrahydrofurane (2 mL) was stirred at room temperature t for 16 h. Then water (1 mL) was added and the mixture was stirred for 3 h at room temperature. The mixture was concentrated and purified by column chromatography (silica, chloroform/MeOH, 80:20) to afford the title compound (10 mg; 23%) as an oil. ¹H-NMR (CDCl₃) δ=2.52 (s, 3 H), 3.90 (s, 2 H), 7.15 (d, 1 H), 7.30 (s, 1 H), 7.50 (d, 1 H).

### Preparative Example 2042

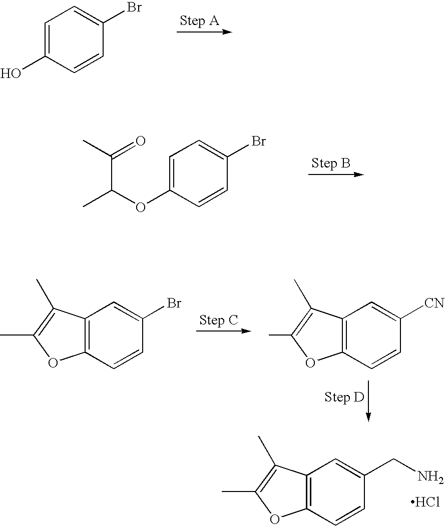 Step A

A solution of 4-bromophenol (3.36 g), 3-chloro-butan-2-one (2.2 mL) and potassium carbonate (4 g) in acetone (40 mL) was refluxed for 3 h. Then additional 3-chloro-butan-2-one and potassium carbonate was added and the mixture was refluxed overnight. The solution was concentrated, dissolved in ethyl acetate and washed with water, 10% aqueous citric acid and brine. The organic phase was dried and evaporated under reduced pressure to give the intermediate (4.88 g; quantitative) as a colourless oil.

Step B

To a solution of phosphorous oxychloride (4.7 mL) was added dropwise the intermediate from step A above (4.88 g) at 100° C. and then the mixture was stirred for 1 h at 100° C. The solution was cooled to room temperature, ice and then ethyl acetate was added and the organic layer was washed with brine and an aqueous saturated sodium hydrogencarbonate solution. The solution was concentrated and purified by column chromatography (silica, cyclohexane) to afford the intermediate (2.55 g; 58% in both steps) as bright yellow solid. ¹H-NMR (CDCl₃) δ=2.10 (s, 3 H), 2.33 (s, 3 H), 7.20-7.30 (m, 2 H), 7.50 (s, 1 H).

Step C

The intermediate from step B above (2.55 g), zinc(II) cyanide (1.0 g) and tetrakis(triphenylphosphine)palladium(0) (653 mg) were dissolved in dry DMF (10 mL), degassed and stirred at 85° C. under argon. After 40 h the mixture was evaporated and diluted with ethyl acetate. The solution was washed with 10% citric acid and brine, dried (MgSO₄), concentrated and purified by column chromatography (silica, cyclohexane/EtOAc, 95:5 to 9:1) to afford the intermediate (1.05 g; 54%) as colourless crystals. ¹H-NMR (CDCl₃) δ=2.18 (s, 3 H), 2.40 (s, 3 H), 7.35-7.50 (m, 2 H), 7.72 (s, 1 H).

Step D

The intermediate from step C above (452 mg), di-tert-butyl dicarbonate (1.2 g) and nickel(II) chloride hexahydrate (64 mg) was dissolved in dry methanol (25 mL) and cooled to 0° C. Then sodium borohydride (600 mg) was added in portions and the ice bath removed. The mixture was vigorously stirred for 4 h, then diethylenetriamine (300 μL) was added and the mixture was concentrated to dryness. The residue was diluted with ethyl acetate, washed with 10% citric acid, saturated sodium hydrogen carbonate and brine, dried (MgSO₄) and concentrated. The solid was suspended in hydrogen chloride (4M solution in dioxane, 10 mL) and was stirred overnight, evaporated, slurried in diethyl ether and filtered to afford the title compound (194 mg; 68%). [M-NH₃Cl]⁺=159.

### Preparative Example 2043

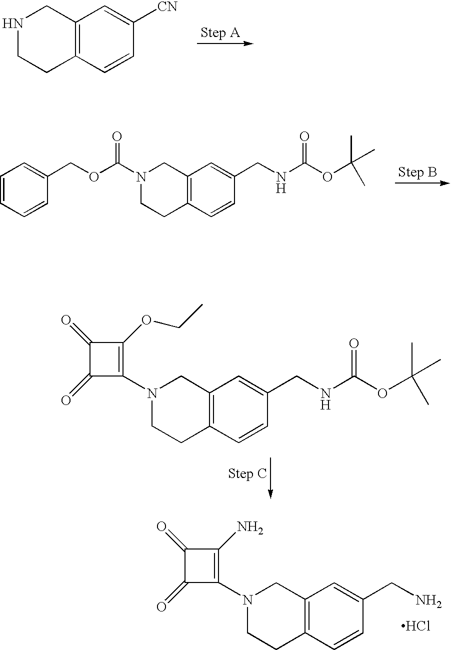 Step A

A solution of 7-cyano-1,2,3,4-tetrahydroisoquinoline (2.75 g), potassium carbonate (3.6 g) and benzylchloroformate (2.7 mL) in THF/water was stirred overnight and then evaporated under reduced pressure. The residue was diluted with ethyl acetate, washed subsequently with 10% citric acid, saturated sodium hydrogen carbonate and brine, dried (MgSO₄) and concentrated. The residue was dissolved in methanol (100 mL) and di-tert-butyl dicarbonate (7.6 g) and nickel(II) chloride hexahydrate (400 mg) was added. The solution was cooled to 0° C., then sodium borohydride (2.6 g) was added in portions. The mixture was allowed to reach room temperature and vigorously stirred overnight, then diethylenetriamine (2 mL) was added and the mixture was concentrated to dryness. The residue was diluted with ethyl acetate, washed with 10% citric acid, saturated sodium hydrogen carbonate and brine, dried (MgSO₄), concentrated and purified by column chromatography (silica, dichloromethane/methanol, 1:0 to 98:2) to afford the intermediate (1.81 g; 26%) as a colourless oil. [MH]⁺=397.

Step B

To a solution of intermediate from step A above (1.81 g) in ethanol (50 mL) was added palladium on charcoal (10 wt %, 200 mg) and then hydrogenated unter normal pressure overnight. The catalyst was filtered off and the solvent was evaporated to 20 mL. Then 3,4-diethoxy-3-cyclobutene-1,2-dione (0.68 mL) and trietylamine (0.5 mL) was added and the mixture was refluxed for 4 h. The solution was concentrated and purified by column chromatography (silica, cyclohexane/EtOAc, 6:4 to 1:1) to afford the intermediate (1.46 g; 83%) as a slowly crystallizing colourless oil.

Step C

To a solution of intermediate from step B above (1.46 g) in ethanol (20 mL) was added ammonia (28% aqueous solution, 100 mL) and the mixture was stirred for 3 h and then evaporated under reduced pressure. The residue was slurried in water, filtered and dried in vaccuo. To the residue was added hydrogen chloride (4M solution in dioxane, 20 mL) and stirred for 14 h, evaporated, suspended in diethyl ether, filtered and dried to afford the title compound (1.08 g; 92%) as an off-white solid. [M-Cl]⁺=258.

### Preparative Example 2044

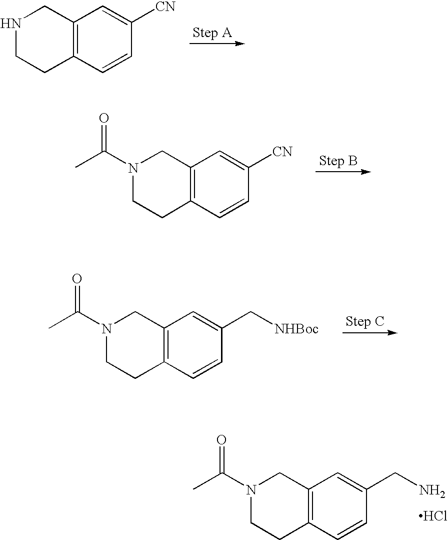 Step A

Commercially available 7-Cyano-1,2,3,4-tetrahydroisoquinoline (158 mg) was dissolved in acetic anhydride (5 mL). Pyridine (0.2 mL) was added and the mixture was stirred overnight. The mixture was concentrated to dryness and the resulting residue was used without further purification for the next step.

Step B

To an ice cooled solution of the title compound from Step A above (200 mg) in dry methanol (20 mL) were added di-tert-butyl dicarbonate (436 mg) and nickel(II) chloride hexahydrate (25 mg), followed by the careful portionwise addition of sodium borohydride (266 mg). The resulting black mixture was stirred for 20 min at 0-5° C. (ice bath), then the ice bath was removed and stirring was continued overnight at room temperature. Then diethylenetriamine (0.4 mL) was added and stirring at room temperature was continued for 30 min. The mixture was concentrated to dryness, ethyl acetate was added and the resulting suspension was washed with aqueous ammonium chloride solution, saturated aqueous sodium hydrogen carbonate and brine, dried (MgSO₄), filtered and concentrated. The resulting residue (250 mg) was used without further purification for the next step.

Step C

A 4M solution of hydrochloric acid in dioxane (5 mL) was added to a solution of the crude title compound from Step B above (250 mg) in dioxane (5 mL). The reaction mixture was stirred at room temperature for 5 h and then concentrated to afford the title compound (230 mg; 99%). [M-Cl]⁺=205.

### Preparative Example 2045

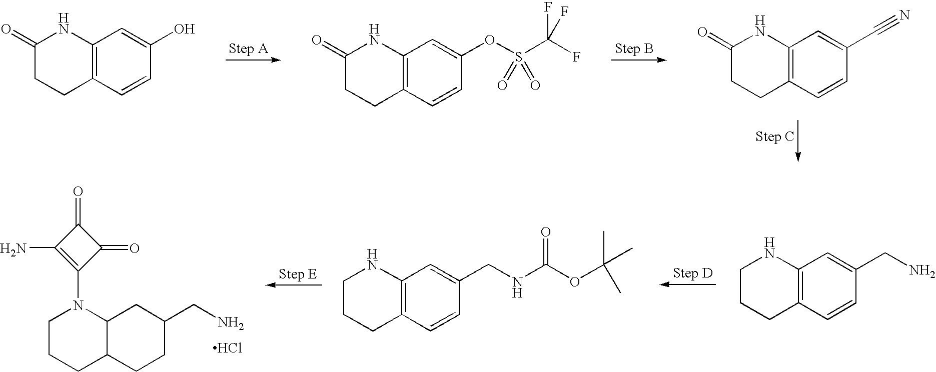 Step A

To a suspension of sodium hydride (95%, 278 mg) in dry tetrahydrofurane (20 mL) was added a suspension of commercially available 7-hydroxy-3,4-dihydro-1H-quinolin-2-one (1.63 g) in dry tetrahydrofurane (20 mL). The resulting suspension was stirred at room temperature for 5 min, then N-phenyl-bis(trifluoromethanesulfonimide) (3.97 g) was added and stirring at room temperature was continued for 2½ h, while the mixture turned from a suspension turned into a clear solution. The mixture was cooled to 0-5° C. (ice bath), hydrolysed by addition of water (40 mL) and extracted with ethyl acetate (3×80 mL). The combined organic layers were washed with saturated aqueous sodium chloride (2×80 mL), dried (MgSO₄), filtered and concentrated. The remaining residue was purified by flash chromatography (silica, cyclohexane/ethyl acetate) to afford the title compound (2.89 g; 98%). [MH]⁺=296.

Step B

The title compound from Step A above (2.89 g), zinc cyanide (930 mg) and tetrakis triphenylphosphine palladium(0) (566 mg) were suspended in dry N,N-dimethylformamide (19.4 mL). The resulting mixture was degassed by three pump/vent cycles with argon and then placed in a preheated oil bath (˜80° C.). After stirring at this temperature for 12½ h the mixture was cooled to room temperature, diluted with water (194 mL) and extracted with ethyl acetate (3×194 mL). The combined organic layers were washed with water (2×194 mL) and brine (194 mL), dried (MgSO₄), filtered and concentrated. The remaining residue was purified by flash chromatography (silica, cyclohexane/ethyl acetate) to afford the title compound (1.38 g; 83%). [MH]⁺=173.

Step C

To a suspension of the title compound from Step B above (1.34 g) in dry tetrahydrofurane (156 mL) was carefully (ice cooling) added lithium aluminium hydride (1.2 g). The resulting mixture was heated to reflux for 18½ h, the cooled to 0 5° C. (ice bath) and carefully hydrolysed by successive addition of water (1.2 mL), 15% aqueous sodium hydroxide (1.2 mL) and water (3.6 mL). The resulting grey suspension was vigorously stirred at room temperature for 1½ h, filtered through a frit and concentrated. The remaining residue was purified by flash (silica, dichloromethane/methanol) to afford the title compound (740 mg; 58%). [MH]⁺=163, [M-NH₂]⁺=146.

Step D

To a solution of the title compound from Step C above (716 mg) in dry dichloromethane (8.8 mL) was added di-tert-butyl dicarbonate (993 mg). The resulting mixture was stirred at room temperature for 16 h, concentrated and purified by flash chromatography (silica, cyclohexane/ethyl acetate) to afford the title compound (785 mg; 68%). [MH]⁺=263.

Step E

If one were to convert the title compound from Step D above as described in the Preparative Example 2025, Step E to Step G, one would obtain the title compound.

### Preparative Example 2046

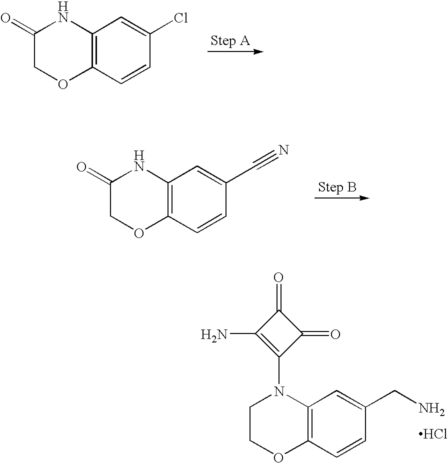 Step A

A suspension of commercially available 6-chloro-4H-benzo[1,4]oxazin-3-one (1.83 g) and copper(I) cyanide (1.81 g) in N-methyl-pyrrolidin-2-one (40 mL) was placed in a preheated oil bath (˜250° C.). After stirring at this temperature for 20 h, the mixture was cooled to room temperature, diluted with water (200 mL) and extracted with ethyl acetate (3×200 mL). The combined organic layers were washed with water (2×200 mL) and brine (200 mL), dried (MgSO₄), filtered and concentrated. The remaining residue was purified by flash chromatography (silica, cyclohexane/ethyl acetate) to afford the title compound (1.08 g; 62%). [MH]⁺=175.

Step B

If one were to convert the title compound from Step A above as described in the Preparative Example 2045, Step C to Step E, one would obtain the title compound.

### Preparative Example 2047

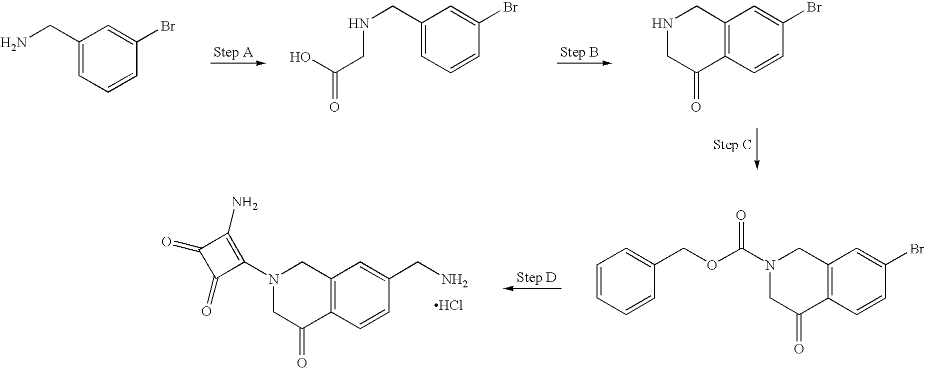 Step A

If one were to treat commercially available 3-bromobenzylamine with bromoacetic acid ethyl ester and saponify the resulting intermediate with aqueous hydrochloric acid as described by A. R. Merrifield et al. (J. Org. Chem. 41, 1976, 2015-2019) one would obtain the title compound.

Step B

If one were to treat the intermediate from Step A above with thionyl chloride and then with aqueous ammonia similar as described by Clemo et al. (J. Chem. Soc., 1939, 1958) one would obtain the title compound.

Step C

If one were to treat the intermediate from Step B above similar as described in the Preparative Example 2028, Step C one would obtain the title compound.

Step D

If one were to treat the intermediate from Step C above similar as described in the Preparative Example 2028, Step D to Step H one would obtain the title compound.

### Preparative Example 2048

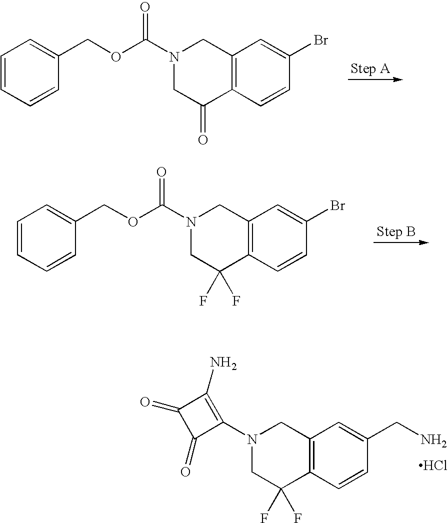 Step A

If one were to treat the intermediate from the Preparative Example 2047; Step C above with ethanethiol and boron trifluoride-acetic acid complex and the resulting dithioacetal with tetrabutylammonium dihydrogentrifluoride and N-iodosuccinimide similar as described by T. Hiyama et al. (Angew. Chem. 117, 2005, 218-234) one would obtain the title compound.

Step B

If one were to treat the intermediate from Step A above similar as described in the Preparative Example 2028, Step D to Step H one would obtain the title compound.

### Preparative Example 2049

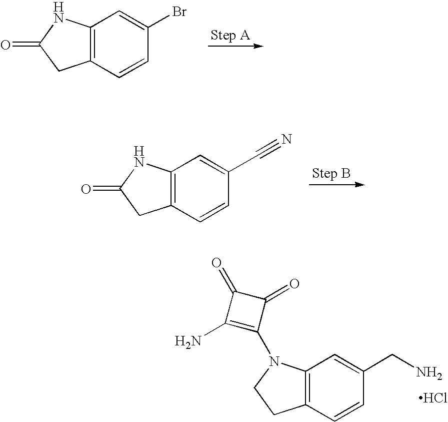 Step A

Commercially available 6-bromoxindole (656 mg), zinc cyanide (288 mg) and tetrakis triphenylphosphine palladium(0) (175 mg) were suspended in dry N,N-dimethylformamide (6 mL). The resulting mixture was degassed by three pump/vent cycles with argon and then placed in a preheated oil bath (˜80° C.). After stirring at this temperature for 15 h the mixture was cooled to room temperature, diluted with water (60 mL) and extracted with ethyl acetate (3×60 mL). The combined organic layers were washed with water (2×60 mL), dried (MgSO₄), filtered and concentrated. The remaining residue was purified by flash chromatography (silica, dichloromethane/methanol) to afford the title compound (385 mg; 81%). [MH]⁺=159.

Step B

If one were to convert the title compound from Step A above as described in the Preparative Example 2045, Step C to Step E, one would obtain the title compound.

### Preparative Example 2050

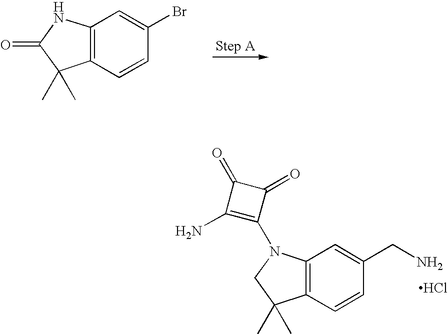 Step A

If one were to convert 6-bromo-3,3-dimethyl-1,3-dihydro-indol-2-one (synthesis described by Atwal et al., J. Med. Chem., 1996, 39, 304-313) as described in the Preparative Example 2049, Step A and Step B, one would obtain the title compound.

### Preparative Example 2051

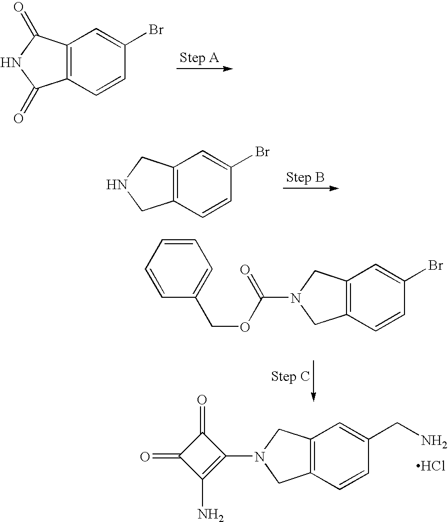 Step A

If one were to reduce commercially available 5-bromo-isoindole-1,3-dione with lithium aluminium hydride in tetrahydrofurane as described in the Preparative Example 2045, Step C, one would obtain the title compound.

Step B

If one were to react the title compound from Step A above with benzyl chloroformate in tetrahydrofurane as described in the Preparative Example 2028, Step C, one would obtain the title compound.

Step C

If one were to convert the title compound from Step B above as described in Preparative Example 2028, Step D to Step H, one would obtain the title compound.

### Preparative Example 2052

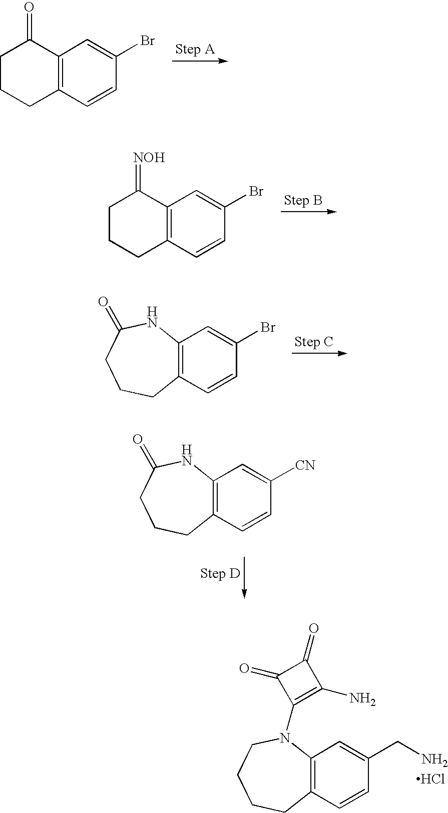 Step A

Commercially available 7-bromo-3,4-dihydro-1(2H)-naphthalenone (3.0 g), hydroxylamine hydrochloride (2.8 g) and sodium acetate (3.4 g) in ethanol (60 mL) were refluxed for 2 h, evaporated, suspended in ethyl acetate and washed with water and brine. After evaporation the title compound (3.27 g; quantitative) was obtained as an off-white solid. [MH]⁺=240/242.

Step B

The intermediate from Step A above (1.51 g) was heated in polyphosphoric acid (35 g) at 150° C. for 4 h. The reaction mixture was poured on ice and extracted three times with ethyl acetate. The combined organic layers were washed with saturated sodium hydrogen carbonate solution and brine, dried and absorbed on silica. Flash chromatography (cyclohexane/ethyl acetate, 8:2) afforded the title compound (1.37 g; 91%) as colourless crystals. ¹H-NMR (CDCl₃) δ=2.15-2.37 (m, 4 H), 2.74 (t, 2 H), 7.06 (d, 1 H), 7.18-7.53 (m, 2 H), 9.13 (s, 1 H); [MH]⁺=240/242.

Step C

The intermediate from Step B above was treated similar as described in the Preparative Example 2025, Step B to obtain the title compound (38%) as tan solid. [MH]⁺=187.

Step D

The intermediate from Step C above was treated similar as described in the Preparative Example 2045, Step C to Step E to obtain the title compound as an off-white solid. [M-Cl]⁺=272.

### Preparative Example 2053

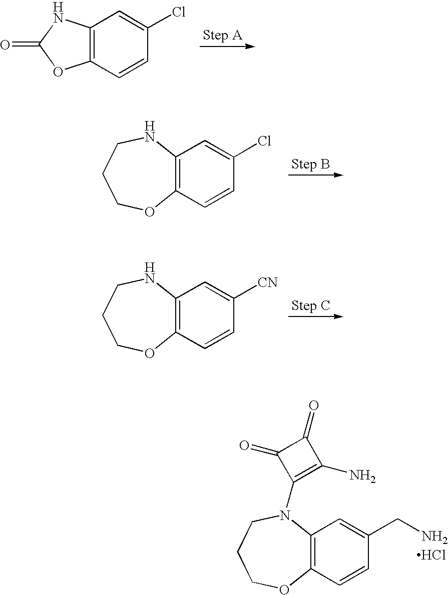 Step A

If one were to treat commercially available 5-chloro-3H-benzooxazol-2-one similar as described by J. Sam et al. (J. Pharm. Sci, 60, 1971, 1370-1375) one would obtain the title compound.

Step B

If one were to treat the intermediate from Step A above with copper(I) cyanide in degassed N-methylpyrrolidin-2-one at 250° C. overnight as described in the Preparative Example 2046, Step A, one would obtain the title compound.

Step C

If one were to treat the intermediate from Step B above similar as described in the Preparative Example 2045, Step C to Step E one would obtain the title compound.

### Preparative Example 2054

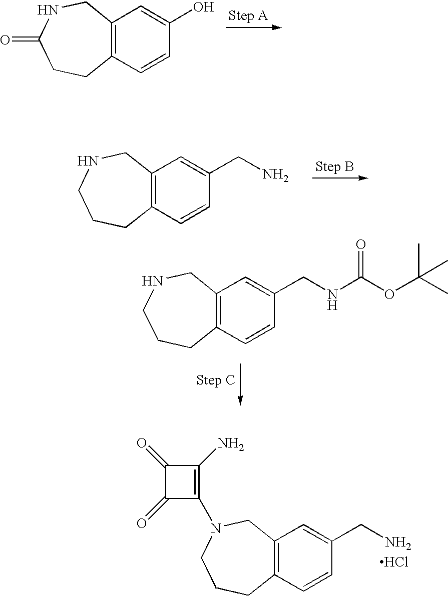 Step A

If one were to treat commercially available 8-hydroxy-1,2,4,5-tetrahydro-3H-2-benzazepin-3-one similar as described in the Preparative Example 2045, Step A to Step C, one would obtain the title compound.

Step B

If one were to treat the intermediate from Step A above similar as described by G. M. Cohen et al. (J. Chem. Soc. Chem. Commun., 1992, 298) one would obtain the the title compound.

Step C

If one were to treat the intermediate from Step B above similar as described in the Preparative Example 2025, Step E one would obtain the title compound.

### Preparative Example 2055

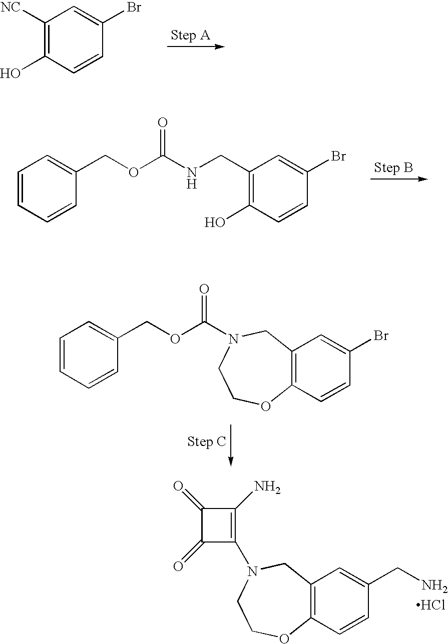 Step A

A solution of commercially available 5-bromo-2-hydroxybenzonitrile (2.00 g), nickel(II) chloride hexahydrate (200 mg) in methanol (50 mL) was cooled to 0° C. and then sodium borohydride (2.25 g) was added in portions and the mixture was allowed to reach room temperature. After 4 h the mixture was cooled to 0° C. and benzyl chloroformate (1.45 mL) in tetrahydrofurane (3 mL) was added. The mixture was allowed to reach room temperature and stirred for 3 h. The mixture was concentrated to dryness and the residue was diluted with ethyl acetate, washed with 10% citric acid and brine, dried (MgSO₄), concentrated and purified by column chromatography (silica, cyclohexane/ethyl acetate, 9:1 to 8:2) to afford the intermediate (2.05 g; 60%) as bright yellow crystals. [MH]⁺=336/338.

Step B

A mixture of the intermediate from Step A above (1.07 g), 1,2-dibromoethane (1.1 mL), Aliquat 336 (0.5 g) and sodium hydroxide (512 mg) in dry dichloromethane (15 mL) and dry acetonitrile (15 mL) was refluxed for 2 h. The mixture was concentrated to dryness and the residue was diluted with ethyl acetate, washed with 10% citric acid and brine, dried (MgSO₄), concentrated and purified by column chromatography (silica, cyclohexane/ethyl acetate, 9:1 to 8:2) to afford an oil, which was dissolved in dry N,N-dimethylformamide (5 mL). The solution was cooled to 0° C. and sodium hydride (60 mg) was added. After stirring overnight, 1N hydrochloric acid was added and the mixture was diluted with ethyl acetate, washed with brine, dried (MgSO₄), concentrated and purified by column chromatography (silica, cyclohexane/ethyl acetate, 9:1 to 8:2) to afford the intermediate (162 mg; 14%) as a clear oil. [MNa]⁺=384/386.

Step C

The intermediate from Step B above treated similar as described in the Preparative Example 2028, Step D to Step H to obtain the title compound as a tan solid. [M-Cl]⁺=274.

### Preparative Example 2056

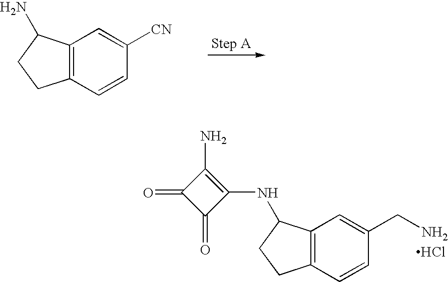 Step A

If one were to treat 3-amino-indan-5-carbonitrile similar as described in the Preparative Example 2043, Step A to Step C one would obtain the title compound.

### Preparative Example 2057

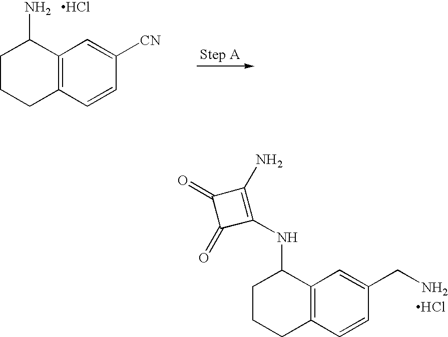 Step A

If one were to treat commercially available 6-cyano-1,2,3,4-tetrahydro-naphthalen-1-yl-ammonium chloride similar as described in the Preparative Example 2043, Step A to Step C one would obtain the title compound.

### Preparative Example 2058

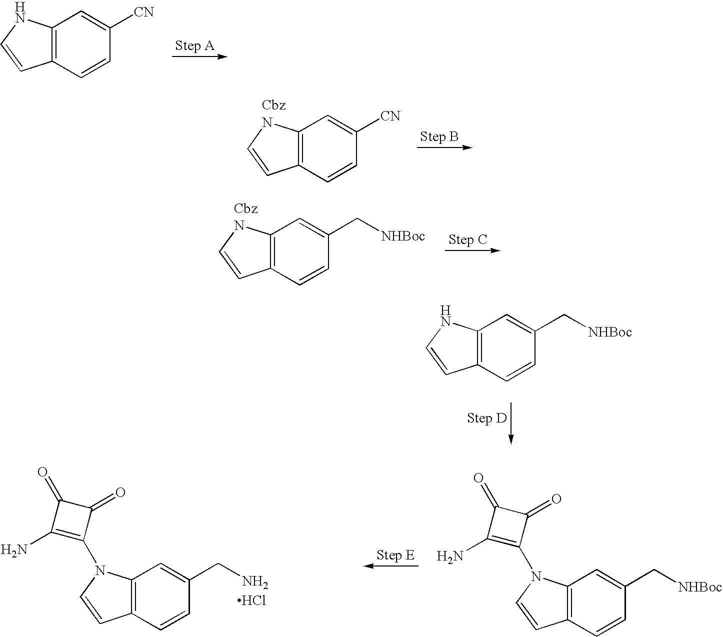 Step A

If one were to stir a solution of commercially available indole-6-carbonitrile, chloro benzylformate and sodium hydride in DMF as described by U. Jacquemard et al. (Tetrahedron, 60, 2004, 10039-10048), one would obtain the title compound.

Step B

If one were to stir a solution of the intermediate from Step A above, di-tert-butyl dicarbonate, nickel(II) chloride hexahydrate and sodium borohydride in dry methanol in an ice bath as described in the Preparative Example 2028, Step E, one would obtain the title compound.

Step C

If one were to stir a solution of the title compound from Step B with palladium on charcoal (10 wt %) in methanol under a hydrogen atmosphere as described in the Preparative Example 2028, Step F, one would obtain the title compound

Step D

If one were to stir the title compound from Step C with 3,4-dichlorocyclobut-3-ene-1,2-dione (synthesized according to E. Arunkumar et al. (J. Am. Chem. Soc., 126, 2004, 6590-6598)) in pyridine at ambient temperature as described by R. M. Anderson et al. (J. Chem. Res. Miniprint, 1985, 3933-3959) and were to quench the reaction mixture with aquous ammonia, one would obtain the title compound.

Step E

If one were to stir the title compounds from Step D above in a 4M solution of hydrochloric acid in dioxane one would obtain the title compound.

### Preparative Example 2059

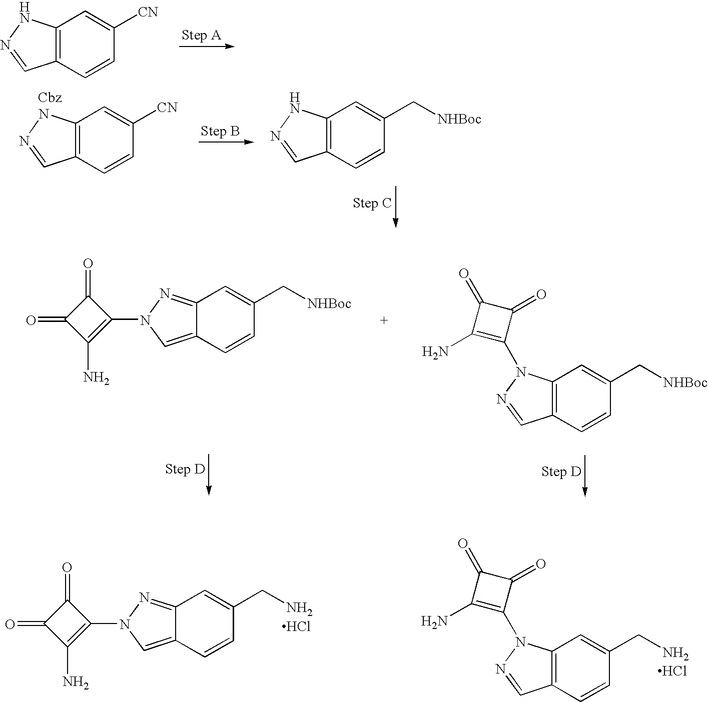 Step A

A solution of commercially available 1H-Indazole-6-carbonitrile (503 mg), chloro benzylformate (560 μL) and potassium carbonate (650 mg) in aqueous tetrahydrofurane was stirred overnight. The mixture was concentrated to dryness, ethyl acetate was added and the resulting solution was washed with a aqueous ammonium chloride solution, saturated aqueous sodium hydrogen carbonate and brine, dried (MgSO₄), filtered and concentrated to afford the title compound (490 mg) as colourless solid.

Step B

To an ice cooled solution of the title compound from Step A above (490 mg) in dry methanol (40 mL) were added di-tert-butyl dicarbonate (783 mg) and nickel(II) chloride hexahydrate (43 mg), followed by the careful portionwise addition of sodium borohydride (470 mg). The resulting black mixture was stirred for 20 min at 0-5° C. (ice bath), then the ice bath was removed and stirring was continued overnight at room temperature. Then diethylenetriamine (0.4 mL) was added and stirring at room temperature was continued for 30 min. The mixture was concentrated to dryness, ethyl acetate was added and the resulting suspension was washed with aqueous ammonium chloride solution, saturated aqueous sodium hydrogen carbonate and saturated aqueous sodium chloride, dried (MgSO₄), filtered and concentrated. The resulting residue was purified by column chromatography (silica, cyclohexane/ethyl acetate=3:2) to afford the title compound (210 mg; 48%) as a colourless solid. [MH]⁺=248.

Step C

If one were to stir the title compound from Step B with 3,4-dichlorocyclobut-3-ene-1,2-dione (synthesized according to E. Arunkumar et al. (J. Am. Chem. Soc., 126, 2004, 6590-6598)) in pyridine at ambient temperature as described by R. M. Anderson et al. (J. Chem. Res. Miniprint, 1985, 3933-3959) and were to quench the reaction mixture with aquous ammonia, one would obtain a mixture of [1-(2-amino-3,4-dioxo-cyclobut-1-enyl)-1H-indazol-6-ylmethyl]-carbamic acid tert-butyl ester and [2-(2-amino-3,4-dioxo-cyclobut-1-enyl)-2H-indazol-6-ylmethyl]-carbamic acid tert-butyl ester, which were to separated by chromatography.

Step D

If one were to stir each of the separated title compounds from Step C in a 4M solution of hydrochloric acid in dioxane one would obtain the title compounds as their hydrochloric acid salts.

### Preparative Example 2060

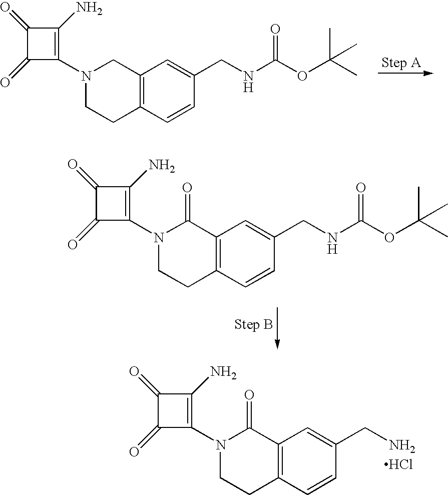 Step A

The intermediate from the Preparative Example 2043, Step C (59 mg) and chromium(XI) oxide (15 mg) was suspended in acetic acid (5 mL) and stirred for 2 h. Then isopropanol was added and the mixture was absorbed on silica. Flash chromatography (dichloromethane/methanol, 99:1 to 98:2 to 96:4) afforded the title compound (38.8 mg; 63%) as tan solid. ¹H-NMR (CDCl₃/CD₃OD) δ=1.38 (s, 9 H), 3.05 (t, 2 H), 4.25 (s, 2 H), 4.48 (d, 2 H), 7.20 (d, 1 H), 7.43 (d, 2 H), 7.89 (s, 1 H); [M-isobutene]⁺=316, [MNa]⁺=394.

Step B

The title compound from Step A above (38.8 mg) was suspended in a 4M solution of hydrochloric acid in dioxane (6 mL) and stirred for 3 h. After evaporation, the title compound (44 mg; quantitative) was obtained as yellow solid. [M-Cl]⁺=272.

### Preparative Example 2061

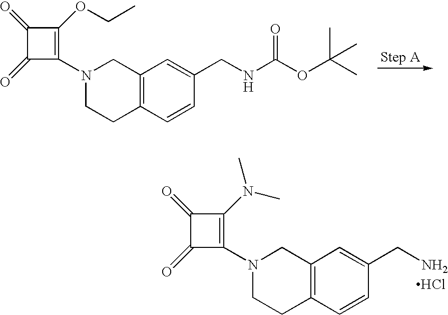 Step A

To a solution of intermediate from the Preparative Example 2043, Step B (100 mg) in ethanol (20 mL) was added dimethyl ammonia (2M solution in tetrahydrofurane, 30 mL) and the mixture was stirred overnight and then evaporated under reduced pressure. To the residue was added hydrogen chloride (4M solution in dioxane, 5 mL) and stirred for 3 h, evaporated and dried to afford the title compound (117 mg) as an off-white solid. [M-Cl]⁺=286.

### Preparative Example 2062

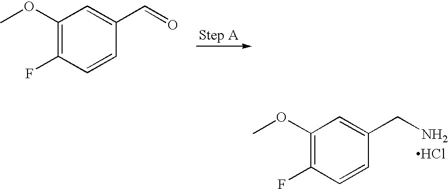 Step A

Commercially available 4-fluoro-3-methoxybenzaldehyde (2.50 g) was dissolved in anhydrous acetonitrile (35 mL). tert-Butylcarbamate (5.70 g) and triethylsilane (5.66 g) were added, forming a suspension. Trifluoracetic acid (5.55 g) was added over 5 min. The resulting clear solution was allowed to stir for 72 h. Volatiles were removed under reduced pressure and the residue taken up in ethyl acetate (40 mL) and washed with water (60 mL) and brine (50 mL). The organic layer was dried over sodium sulfate, concentrated, and the residue purified by column chromatography on silica (hexane/ethyl acetate, 2:1). The Boc group was removed by dissolving the protected amine in a 4M solution of hydrochloric acid in dioxane (10 mL) for 1 h. The resulting slurry was diluted with ethyl ether (15 mL) and hexane (15 mL) and the title compound (2.6 g; 80%) was dried under vacuum. [M-NH₃Cl]⁺=175.

### Preparative Example 2063

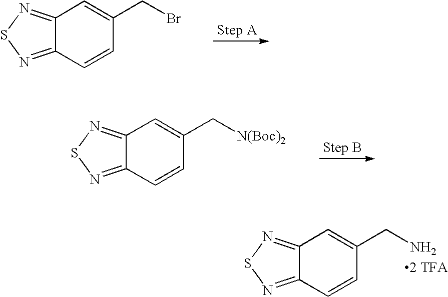 Step A

Commercial available 5-bromomethyl-benzo[1,2,5]thiadiazole (115 mg) in DMF (1 mL) was added to a stirred solution of the potassium salt of carbamic acid bis-tert-butylester in DMF (2 mL) (prepared according to J. Chem. Soc., Perkin Trans. 1, 1983, 2983-2985). Stirring was continued at 50° C. for 2 h. The solvent was removed in vaccuo, and the residue was diluted with ethyl acetate and washed with saturated aqueous NaHCO₃, dried and concentrated. The crude product was used without purification in the next step. [MH]⁺=366.

Step B

The title compound from Step A above (180 mg) was dissolved in trifluoroacetic acid (2 mL). After stirring for 1 h at room temperature, the solvent was evaporated to give the trifluoracetic acid salt of the title compound (180 mg; quantitative). [MH]⁺=166.

### Preparative Example 2064

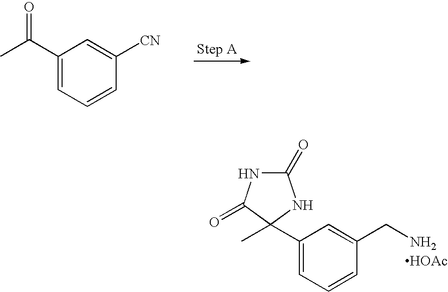 Step A

3-Actyl-benzonitrile (2.1 g), sodium cyanide (1.08 g) and ammonium carbonate (6.95 g) were suspended in ethanol (20 mL) and water (20 mL) and heated to 70° C. until complete. Typical aqueous workup and concentration gave the intermediate. Hydrogenation with palladium on carbon (10%) in ethanol and acetic acid yielded the title compound (2.04 g; 50%).

### Preparative Example 2065

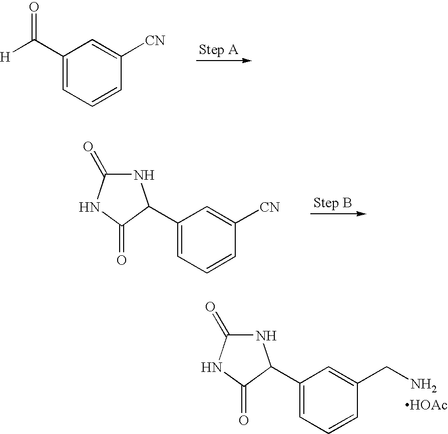 Step A

To 3-cyanobenzaldehyde (263 mg) in 50% aqueous ethanol (12 mL) was added potassium cyanide (130 mg) and ammonium carbonate (769 mg). The reaction mixture was heated to 55° C. and kept at the temperature overnight. The solution was allowed to cool down and the precipated solid was filtered off. The filtrate was concentrated and extracted with ether (3×10 mL). The combined organic layer was washed with brine, dried over magnesium sulfate and concentrated to give a crude oil. The crude product was purified by silica gel chromatography to give the title compound (347 mg; 86%) as colourless solid. [MH]⁺=202.

Step B

The title compound from Step A above (347 mg) was dissolved in ethanol and palladium on carbon (10%; 200 mg) and 50% aqueous acetic acid (2 mL) was added. The solution was hydrogenated (50 psi) overnight. The solution was filtered and concentrated to give the title compound as colourless solid foam in quantative yield. [M-OAc]⁺=206.

### Preparative Example 2066

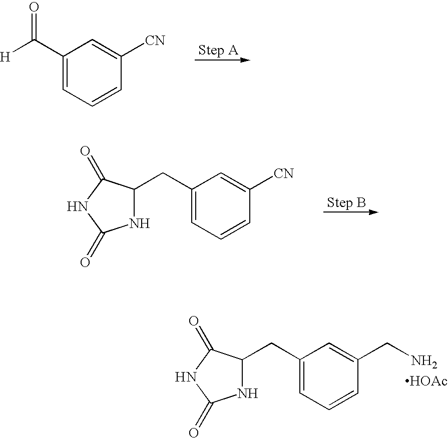 Step A

The mixture of 3-cyanobenzaldehyde (262 mg), hydantoin (220 mg), potassium acetate (380 mg) in acetic acid (2 mL) was heated to reflux for 3 h. The solution was poured on ice (20 g). The colourless precipitate was collected and washed with ice water. The solid was dried in vaccuo to give the title compound as a yellow solid. [MH]⁺=214.

Step B

The title compound from Step A above was treated as described in the Preparative Example 2065 Step B. [M-Ac]⁺=214.

### Preparative Example 2067

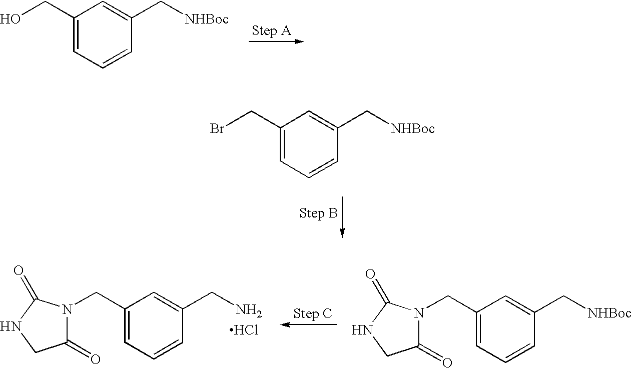 Step A

The solution of Boc-aminomethylbenzyl alcohol (237 mg), triphenylphosphine (485 mg) and carbon tetrabromide (491 mg) in dichloromethane (10 mL) was stirred at room temperature overnight and then was concentrated. The crude mixture was purified by silica gel chromatography to give the title compound (200 mg; 67%).

Step B

The mixture of the title compound from Step A above (90 mg), hydantoin (36 mg), potassium carbonate (150 mg) and tetrabutylammonium iodide (1 mg) in N,N-dimethylformamide was stirred at room temperature for 24 h. The solution was concentrated to dryness, and then dissolved in ethyl acetate (20 mL). The solution was washed with water and brine, dried over magnesium sulfate and concentrated to give the title compound (90 mg) as a yellow solid.

Step C

To the title compound from Step B above was added hydrogen chloride in dioxane (4M, 1 mL). The solution was stirred for 1 h and diluted with diethyl ether (10 mL). The precipitate was collected and washed with additional diethyl ether (2 mL) to afford the title compound as a colourless solid.

### Preparative Example 2100

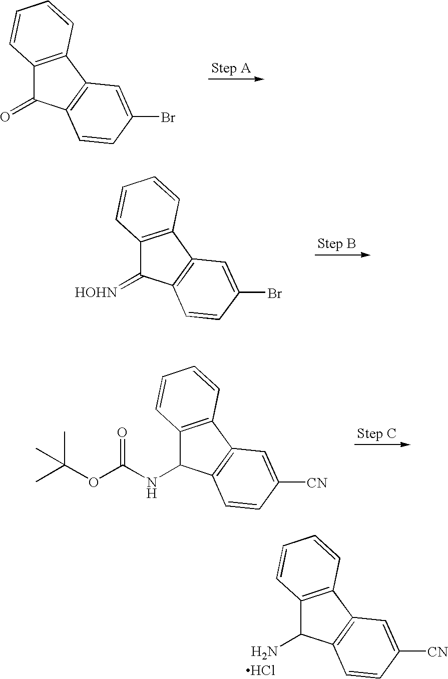 Step A

A suspension of 3-bromo-fluoren-9-one (C. F. Koelsch, J. Amer. Chem. Soc., 1944, 1983-1984) (1.08 g), sodium hydrogen carbonate (3.5 g) and hydroxylamine hydrochloride (3.5 g) in ethanol (40 mL) was stirred at 80° C. overnight. The solvent was evaporated, the residue dissolved in ethyl acetate and washed with brine. Evaporation afforded the intermediate (1.13 g; 99%) as bright yellow crystals.

Step B

The intermediate from Step A above (1.13 g) was dissolved in dry diethyl ether (30 mL) and cooled to 0° C. Then lithium aluminiumhydride (1N solution in diethyl ether, 20 mL) was added. After 30 min at 0° C., the solution was refluxed for 90 min. After addition of water (0.8 mL), 15% aqueous sodium hydroxide (0.8 mL) and again water (2.4 mL), the precipitate was filtered off. The remaining liquid was evaporated under reduced pressure. The oil was dissolved in dry tetrahydrofurane (20 mL) and treated with di-tert-butyl dicarbonate (1.09 g) and triethylamine (0.66 mL). After 16 h the mixture was evaporated and diluted with ethyl acetate. The solution was washed with 10% citric acid and brine, dried (MgSO₄) and concentrated. To the residue was added zinc(II) cyanide (350 mg) and tetrakis(triphenylphosphine)palladium(0) (230 mg) and dry DMF (20 mL). The solution was degassed and stirred at 100° C. under argon. After 40 h the mixture was evaporated and diluted with ethyl acetate. The solution was washed with 10% citric acid and brine, dried (MgSO₄), concentrated and purified by column chromatography (silica, cyclohexane/EtOAc, 9:1 to 8:2) to afford the intermediate (239 mg; 19%) as colourless crystals. [MH]⁺=307, [MNa]⁺=329.

Step C

To intermediate from Step B above (127 mg) was added hydrogen chloride (4M solution in dioxane, 8 mL) and stirred for 17 h, filtered and dried to afford the title compound (83 mg; 82%) as a colourless solid. [M-Cl]⁺=207, [MNa]⁺=229, [M-NH₃Cl]⁺=189.

### Preparative Example 2101

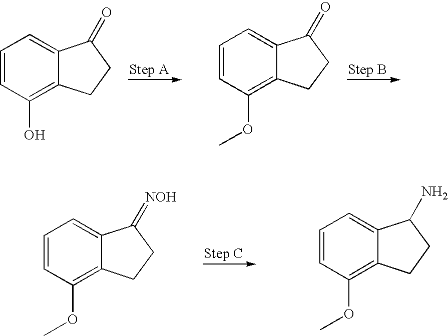 Step A

A mixture of 4-hydroxy-indan-1-one (125 mg), K₂CO₃ (350 mg), methyl iodide (263 μL) in DMF (4 mL) was stirred at 50° C. for 3 h and then poured into 1N hydrochloric acid (20 mL) and washed with Et₂O (4×10 mL). The combined organic layers were dried over MgSO₄, filtered and concentrated to afford the intermediate as a clear oil (131 mg; 96%). [MH]⁺=163.

Step B

A mixture of intermediate from Step A above (131 mg), NH₂OH.HCl (62 mg), and NaOAc (73.2 mg) in MeOH (4 mL) was allowed to stir for 16 h at 22° C. Water (10 mL) was added and the resulting precipitate was filtered and washed three times with water (2 mL) to afford the intermediate as a colourless solid (133 mg; 91%). [MH]⁺=178.

Step C

To a mixture of intermediate from Step B above (133 mg) in Et₂O (2 mL) at −78° C. under an atmosphere of argon was slowly added a 1M solution of lithium aluminum hydride in Et₂O (4.0 mL). The mixture was heated to reflux (40° C.) and allowed to stir for 5 h. The mixture was cooled to 0° C. and water (0.15 mL), 15% aqueous NaOH (0.15 mL) and water (0.45 mL) were carefully and sequentially added. The resulting mixture was filtered through Celite® and the filtrate was concentrated to give the title compound as a clear oil (71.6 mg; 40%). [M-NH₂]⁺=147.

### Preparative Example 2102

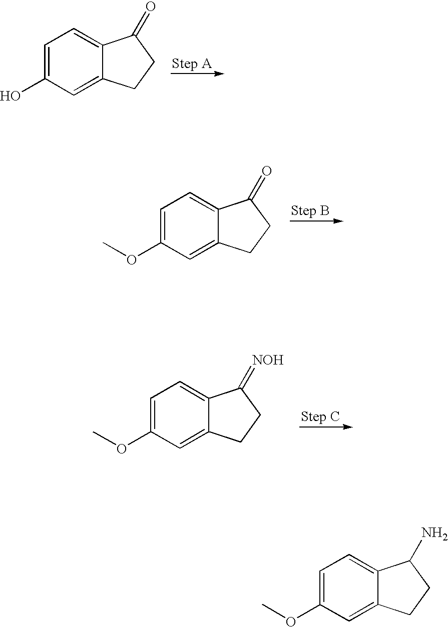 Step A

A mixture of 5-hydroxy-indan-1-one (125 mg), K₂CO₃ (350 mg), methyl iodide (263 μL) in DMF (4 mL) was stirred at 50° C. for 3 h and poured into 1N hydrochloric acidl (20 mL) and washed with Et₂O (4×10 mL). The combined organic layers were dried over MgSO₄, filtered and concentrated to afford the intermediate as a clear oil (44.7 mg; 33%). [MH]⁺=163.

Step B

A mixture of intermediate from Step A above (44.7 mg), NH₂OH.HCl (21 mg), and NaOAc (25 mg) in MeOH (1 mL) was allowed to stir for 16 h at 22° C. Water (5 mL) was added and the resulting precipitate was filtered and washed three times with water (1 mL) to afford the intermediate as a colourless solid (44 mg; 97%). [MH]⁺=178.

Step C

To a mixture of intermediate from Step B above (44.7 mg) in Et₂O (1 mL) at −78° C. under an atmosphere of argon was slowly added a 1M solution of lithium aluminum hydride in Et₂O (1.35 mL). The mixture was heated to reflux (40° C.) and allowed to stir for 5 h. The mixture was cooled to 0° C. and water (0.05 mL), 15% aqueous NaOH (0.05 mL), and water (0.15 mL) were carefully and sequentially added. The resulting mixture was filtered through Celite® and the filtrate was concentrated to give the title compound as a clear oil (27 mg; 61%). [M-NH₂]⁺=147.

### Preparative Example 2103

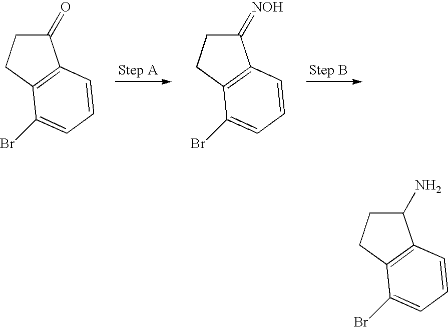 Step A

Commercially available 4-bromo-2,3-dihydroinden-1-one (514 mg), hydroxylamine hydrochloride (187 mg) and sodium acetate (220 mg) were added to methanol (12 mL) and stirred at room temperature. After 15 h the mixture was diluted with H₂O (50 mL). The intermediate (517 mg, 94%) was collected through filtration as colourless solid. [MH]⁺=226/228.

Step B

The intermediate from Step A above (517 mg) was dissolved in anhydrous diethylether (7 mL). The solution was cooled to −78° C., and lithium aluminum hydride (1M in Et₂O, 11.5 mL) was added dropwise. The mixture was heated to reflux and stirred for 15 h, then cooled down to −30° C. Water (0.5 mL) and a 1M aqueous sodium hydroxide solution (1 mL) were added to the mixture slowly. The reaction mixture was warmed up to room temperature and filtered through Celite®. The filtrate was concentrated to afford the title compound (387 mg) as a solid. [MH]⁺=212/214.

### Preparative Example 2104

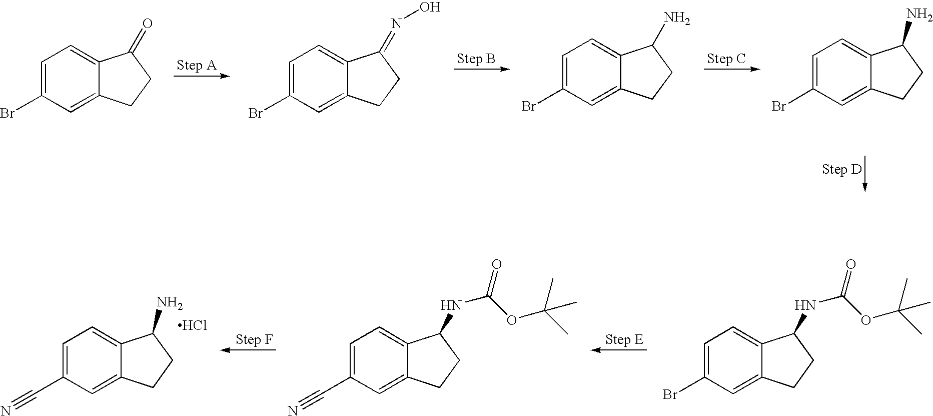 Step A

A mixture of commercially available 5-bromo-indan-1-one (1.76 g), hydroxylamine hydrochloride (636 mg) and sodium acetate (751 mg) in methanol (40 mL) was allowed to stir for 16 h at room temperature. Water (100 mL) was added and the resulting precipitate was filtered and washed with water (3×20 mL) to afford the title compound (1.88 g; >99%) as a colourless solid. [MH]⁺=226/228.

Step B

To a solution of the title compound from Step A above (1.88 g) in diethyl ether (20 mL) at −78° C. under an atmosphere of argon was slowly added a 1M solution of lithium aluminum hydride in diethyl ether (42.4 mL). The mixture was heated to reflux (40° C.) and allowed to stir for 5 h. The mixture was cooled to 0° C. and water (1.6 mL), 15% aqueous sodium hydroxide (1.6 mL) and water (4.8 mL) were carefully and sequentially added. The resulting mixture was filtered through Celite ® and the filtrate was concentrated to give the title compound (1.65 g; 94%) as a clear oil. [MH]⁺=212/214.

Step C

To a boiling solution of the title compound from Step B above (1.13 g) in methanol (2.3 mL) was added a hot solution of commercially available N-acetyl-L-leucine (924 mg) in methanol (3 mL). The solution was allowed to cool to room temperature, which afforded a white precipitate. The solid was separated from the supernatant and washed with methanol (2 mL). The solid was recrystalized two times from methanol. To the resulting solid were added 10% aqueous sodium hydroxide (20 mL) and diethyl ether (20 mL). Once the solid was dissolved, the organic layer was separated and the aqueous layer was washed with diethyl ether. The combined organic layers were dried (MgSO₄), filtered and concentrated to give the title compound (99 mg; 18%) as a clear oil. [MH]⁺=212/214.

Step D

To a solution of the title compound from Step C above (300 mg), di-tert-butyl dicarbonate (370 mg) and triethylamine (237 μL) in tetrahydrofurane (10 mL) was allowed to stir for 16 h at room temperature. The solution was concentrated and the remaining residue was purified by chromatography (silica, hexanes/ethyl acetate) to give the title compound (460 mg; >99%) as a clear oil. [(M-isobutene)H]⁺=256/258, [MNa]⁺=334/336.

Step E

A mixture of the title compound from Step D above (460 mg), tetrakis triphenylphosphinepalladium (89 mg), zinc cyanide (200 mg) in N,N-dimethylformamide (5 mL) under an atmosphere of argon in a sealed vial was allowed to stir for 18 h at 110° C. The mixture was allowed to cool to room temperature before diethyl ether (20 mL) and water (20 mL) were added. The separated aqueous layer was washed with diethyl ether (4×10 mL). The combined organic layers were washed with water (3×10 mL) and brine (10 mL), dried (MgSO₄), filtered and concentrated. The resulting residue was purified by chromatography (silica, hexanes/ethyl acetate) to afford the title compound (170 mg; 47%) as a clear oil. [MH]⁺=259, [MNa]⁺'281.

Step F

To the title compound from Step E above (170 mg) was added a 4M solution of hydrochloric acid in dioxane (2 mL). The resulting solution was allowed to stir for 3 h at room temperature at which time a precipitate had formed. The mixture was concentrated to give the title compound (128 mg; >99%). [M-Cl]⁺=159.

### Preparative Example 2105

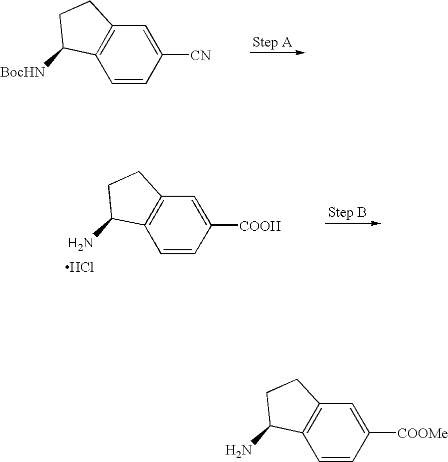 Step A

The title compound from the Preparative Example 2104, Step E above (1.0 g) was suspended in 6N hydrochloric acid (50 mL) and heated to 110-112° C. for 20 h upon which the solution became homogeneous. The solvent was removed under reduce pressure to give the intermediate. [M-Cl]⁺=178.

Step B

The intermediate from Step A above was dissolved in anhydrous MeOH (150 mL) and saturated with anhydrous hydrogen chloride gas. The reaction mixture was then heated to reflux for 20 h. After cooling to room temperature, the solvent was removed under reduced pressure to give an oil. The oil was taken up in dichloromethane and washed with saturated NaHCO₃. The organic phase was separated and dried over MgSO₄, filtered and concentrated to give the title compound (0.66 g, 89% over two steps) as an oil which slowly crystallized into a light brown solid.

### Preparative Example 2106

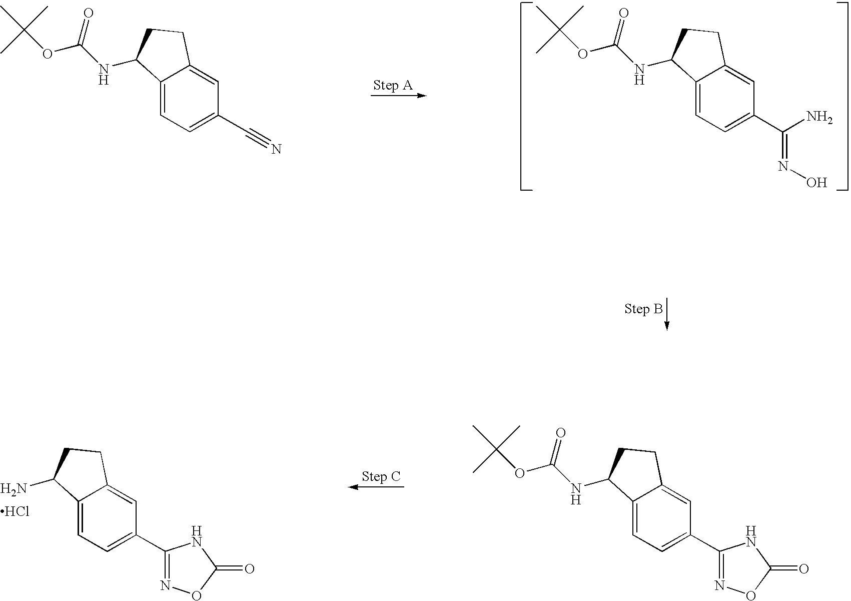 Step A

To a solution of hydroxylamine hydrochloride (2.78 g) in dry methanol (100 mL) was added sodium methoxide (30wt % in methanol, 7.27 mL). The resulting white suspension was stirred at room temperature for 15 min, then a solution of the title compound from Preparative Example 2104, Step E (5.17 g) in dry methanol (100 mL) was added and the mixture was heated to reflux for 20 h and then cooled to room temperature. The obtained solution of the title compound (complete conversion checked by HPLC/MS, [MH]⁺=292) was directly used for Step B below.

Step B

To the solution obtained in Step A above were added successively diethyl carbonate (48.2 g) and sodium methoxide (30wt % in methanol, 7.27 mL). The resulting mixture was heated to reflux for 24 h and then concentrated. To the remaining material was added 1M aqueous ammonium chloride solution (200 mL) and the resulting aqueous mixture was extracted with methanol/dichloromethane (40:60, 500 mL) and dichloromethane (3×200 mL). The combined organic layers were dried (MgSO₄), concentrated and purified by flash chromatography (silica, dichloromethane/methanol) to afford the title compound as a colourless solid (3.89 g; 61%). [MNa]⁺=340.

Step C

The title compound from Step B above (991 mg) was suspended in a 4M solution of hydrochloric acid in dioxane (12.5 mL). The reaction mixture was stirred for 1 h at room temperature and then concentrated to afford the title compound (785 mg; 99%). [M-Cl]⁺=218.

### Preparative Example 2107

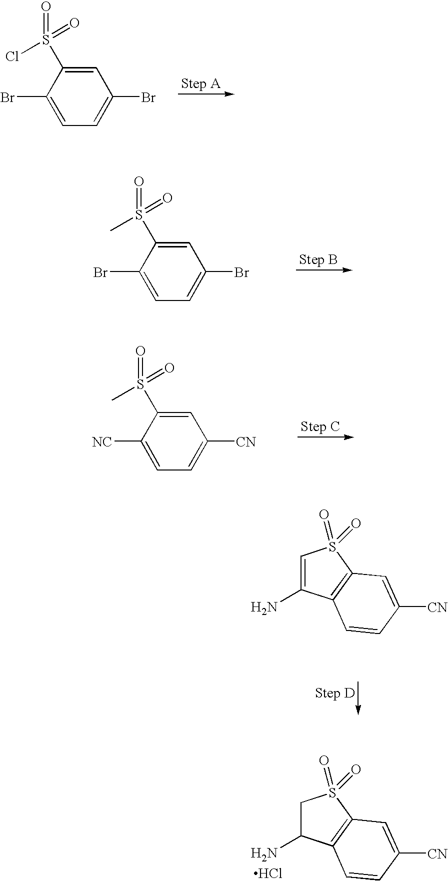 Step A

A suspension of 2,5-dibromobenzenesulfonyl chloride (1.0 g), sodium sulfite (0.46 g) and sodium hydroxide (0.27 g) in water (10 mL) was heated to 70° C. for 5 h. To the cooled solution was added methyl iodide (4 mL) and methanol. The biphasic system was stirred vigorously at 50° C. overnight, then evaporated and suspended in water. Filtration afforded the intermediate (933 mg; 99%) as colourless needles. [MH]⁺=313/315/317, [MNa]⁺=335/337/339.

Step B

The intermediate from Step A above (8.36 g) and copper(I) cyanide (7.7 g) in degassed N-methylpyrrolidone (30 mL) was heated in a sealed tube to 160° C. overnight. After evaporation of the solvent the residue was absorbed on silica and purified by column chromatography (silica, cyclohexane/EtOAc, 6:4 to 4:6) to afford the intermediate (1.08 g; 20%) as beige crystals.

Step C

The intermediate from Step B above (980 mg) and 1,8-diazabicyclo[5.4.0]undec-7-ene (0.72 mL) was heated in degassed dimethylsulfoxide to 50° C. for 45 min under argon. To the solution was added ethyl acetate and then washed with 10% citric acid and brine, dried (MgSO₄), concentrated and purified by column chromatography (silica, cyclohexane/EtOAc, 4:6 to 3:7) to afford the intermediate (694 mg; 71%) as a bright yellow solid. ¹H-NMR (CD₃CN) δ=5.70 (s, 2 H), 5.75 (br s, 2 H), 7.72 (d, 1 H), 8.00-8.10 (m, 2 H).

Step D

To a solution of the intermediate from Step C above (892 mg) in DMF (10 mL) was added palladium on charcoal (10 wt %, 140 mg) and then hydrogenated unter normal pressure for 2 h. The catalyst was filtered off and to the solvent was added di-tert-butyl dicarbonate (440 mg) and stirred overnight. The solvent was evaporated and diluted with ethyl acetate. The solution was washed with 10% citric acid and brine, dried (MgSO₄) and concentrated. Purification by column chromatography (silica, cyclohexane/EtOAc, 6:4) afforded a colourless solid, which was stirred in hydrogen chloride (4M solution in dioxane, 20 mL) overnight, evaporated and dried to afford the intermediate (69 mg; 8%) as colourless crystals. [M-Cl]⁺=209.

### Preparative Example 2108

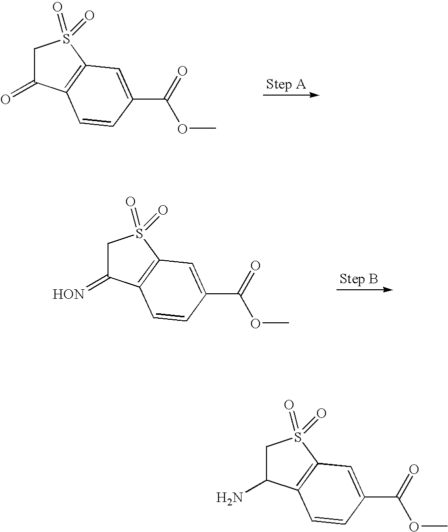 Step A

A solution of 1,1,3-trioxo-2,3-dihydro-1H-1λ⁶-benzo[b]thiophene-6-carboxylic acid methyl ester (M. Baumgarth et al., J. Med. Chem., 1998, 41, 3736-3747) (286 mg), sodium acetate (490 mg) and hydroxylamine hydrochloride (490 mg) in dry methanol (20 mL) was refluxed for 2½ h. The solvent was evaporated, the residue dissolved in ethyl acetate and washed with brine. Evaporation afforded the intermediate (302 mg; 99%) as an off-white solid. ¹H-NMR (DMSO): δ=3.90 (s, 3 H), 4.57 (s, 2 H), 8.04 (d, 1 H), 8.25-8.28 (m, 2 H), 12.62 (s, 1 H).

Step B

The intermediate from Step A above (170 mg) was dissolved in methanol (50 mL) and heated to 60° C. Then zinc dust (500 mg) and 6N hydrochloric acid (5 mL) was added in portions over 30 min. The mixture was cooled, filtered and evaporated. After dilution with ethyl acetate, the solution was washed with a saturated sodium hydrogen carbonate solution and brine, dried (MgSO₄) and concentrated to afford the intermediate (128 mg; 80%) as yellow oil. [MH]⁺=242, [MNa]⁺=264.

### Preparative Example 2109

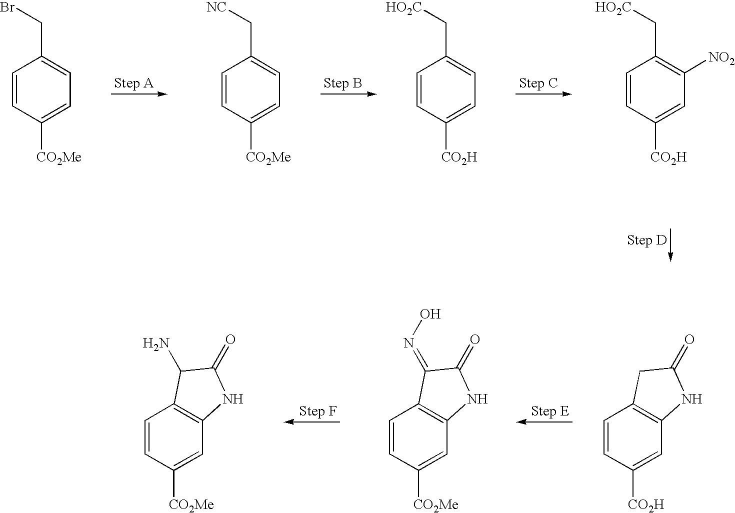 Step A

Commercially available 4-bromomethylbenzoic acid methyl ester (10.0 g) was dissolved in ethanol (40 mL). A potassium cyanide solution (5.63 g in 8 mL water) was added dropwise over 15 min. The resulting suspension was heated to reflux for 3 h. Volatiles were removed under reduced pressure and the residue dissolved in diethylether and water. The organic layer was concentrated to a dark oil which was purified by column chromatography (5% ethyl ether in dichloromethane) to give the intermediate (6.0 g). [MH]⁺=176.

Step B

The intermediate from Step A above (6.0 g) was dissolved in 50% aqueous sulfuric acid (40 mL). The solution was heated at 125° C. overnight, resulting in precipitation of a brown solid after cooling. The mixture was filtered and the solid recrystallized from hot glacial acetic acid (45 mL). The product was filtered, washed with a small amount of water and dried under vacuum to give the intermediate (4.15 g) as an off-white solid. [MH]⁺=181.

Step C

The intermediate from Step B above (4.15 g) was added in small portions over 4 h to a mixture of fuming nitric acid (11 mL) and sulfuric acid (15 mL) at 0° C. After addition was complete, the reaction was warmed to room temperature and poured onto crushed ice. The resulting precipitate was filtered, washed with cold water and dried under vacuum to give the intermediate (4.5 g). [MNa]⁺=248.

Step D

The intermediate from Step C above was dissolved in methanol (50 mL) and ammonium hydroxide (5 drops) and water (2 mL) were added. The solution was cooled to 0° C., and palladium on charcoal (10 wt %, 250 mg) was added. The flask was fitted with a hydrogen balloon and stirred for 2 h. The balloon was refilled with hydrogen and the reaction was stirred overnight. The resulting precipitate was dissolved by the addition of 1N sodium hydroxide solution and the solution filtered through Celite®. Volatiles were removed under reduced pressure to give the intermediate (2.58 g) as a tan solid. [MH]⁺=178.

Step E

The intermediate from Step D was esterified by heating in acidic methanol overnight. The resulting solution was concentrated under vacuum and the residue was dissolved in hot ethanol (75 mL). Methanol (20 mL) was added to redissolve some material that precipitated as the solution cooled. Sodium nitrite (1.50 g) was added, followed by concentrated hydrochloric acid (5 mL). The reaction was stirred for 2 h, and a further concentrated hydrochloric acid (2 mL) was added. The reaction was stirred overnight. Volatiles were removed under reduced pressure, water added, and the resulting yellow orange product was isolated by filtration and dried under vacuum to give the intermediate (2.1 g) as a yellow orange solid. [MH]⁺=221.

Step F

The intermediate from Step E (1.00 g) was dissolved in methanol (50 mL) and concentrated hydrochloric acid (1 mL). Palladium on charcoal (10 wt %, 250 mg) was added as a slurry in methanol (5 mL) and the reaction was placed on a Parr shaker-type hydrogenation apparatus at 50 psi hydrogen. After 3 h, the solution was filtered through Celite® and volatiles were removed under reduced pressure. The resulting tan solid was washed with ether and dried under vacuum to afford the intermediate (900 mg). [MH]⁺=207.

### Preparative Example 2110

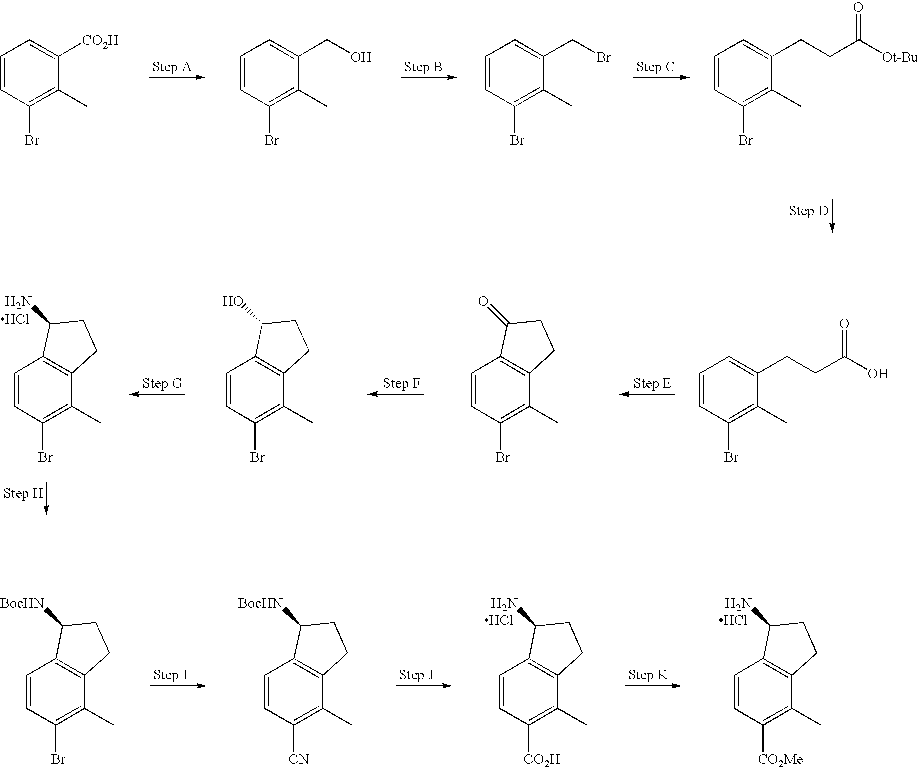 Step A

3-Bromo-2-methyl-benzoic acid (20.0 g) was dissolved in anhydrous THF (200 mL) under nitrogen and the reaction vessel was cooled to 0° C. in an ice bath. To this cooled solution was added BH₃.THF complex (1M in THF, 140 mL) dropwise over a 3 h period. Once gas evolution had subsided, the reaction mixture was warmed to room temperature and stirred for an additional 12 h. The mixture was then poured into 1N hydrochloric acid (500 mL) cooled with ice and then extracted with Et₂O (3×150 mL). The organic extracts were combined, dried over anhydrous MgSO₄, filtered, and then concentrated to afford the intermediate (18.1 g; 97%) as a colourless solid. ¹H-NMR (CDCl₃) δ=2.40 (s, 3 H), 4.70 (s, 2 H), 7.10 (t, 1 H), 7.30 (d, 1 H), 7.50 (d, 1 H).

Step B

The intermediate from Step A above (18.1 g) was dissolved in anhydrous CH₂Cl₂ (150 mL) under nitrogen and the reaction vessel was cooled to 0° C. in an ice bath. To this cooled solution was added PBr₃ (5.52 mL) over a 10 min period. Once the addition was complete, the reaction mixture was warmed to room temperature and stirred for an additional 12 h. The mixture was cooled in an ice bath and quenched by the dropwise addition of MeOH (20 mL). The organic phase was washed with saturated NaHCO₃ (2×150 mL), dried over anhydrous MgSO₄, filtered, and then concentrated to afford the intermediate (23.8 g; 97%) as viscous oil.

¹H-NMR (CDCl₃) δ=2.50 (s, 3 H), 4.50 (s, 2 H), 7.00 (t, H), 7.25 (d, 1 H), 7.50 (d, 1 H).

Step C

t-Butyl acetate (12.7 mL) was dissolved in anhydrous THF (200 mL) under nitrogen and the reaction vessel was cooled to −78° C. in a dry ice/acetone bath. To this cooled solution was added dropwise lithium diispropylamide (1.5M in cyclohexane, 63.0 mL) and the mixture was allowed to stir for an additional 1 h upon which a solution of intermediate from Step B above (23.8 g) was added in THF (30 mL). Once the addition was complete, the reaction mixture was gradually warmed to room temperature over a 12 h period. The mixture was concentrated and the remaining viscous oil was dissolved in Et₂O (300 mL), washed with 0.5N hydrochloric acid (2×100 mL), dried over anhydrous MgSO₄, filtered, and then concentrated to afford the intermediate (21.5 g; 80%) as a pale-yellow viscous oil. ¹H-NMR (CDCl₃) δ=1.50 (s, 9 H), 2.40 (s, 3 H), 2.50 (t, 2 H), 3.00 (t, 2 H), 7.00 (t, 1 H), 7.25 (d, 1 H), 7.50 (d, 1 H).

Step D

The intermediate from Step C above (21.5 g) was combined with polyphosphoric acid (250 g) and placed in a 140° C. oil bath for 10 min while mixing the thick slurry occasionally with a spatula. To this mixture was then added ice water (1 L) and the mixture was stirred for 2 h. The mixture was then filtered and the solid was washed with H₂O (2×100 mL) and dried to afford the intermediate (16.7 g; 96%). ¹H-NMR (CDCl₃) δ=2.40 (s, 3 H), 2.65 (t, 2 H), 3.00 (t, 2 H), 7.00 (t, 1 H), 7.20 (d, 1 H), 7.50 (d, 1 H).

Step E

The intermediate from Step D above (11.6 g) was dissolved in anhydrous CH₂Cl₂ (100 mL) under nitrogen and the reaction vessel was cooled to 0° C. in an ice bath. To this mixture was added dropwise oxalyl chloride (12.0 mL) and the mixture was stirred for 3 h after which the mixture was concentrated under reduced pressure. The remaining dark residue was dissolved in anhydrous CH₂Cl₂ (300 mL) and to this mixture was added AlCl₃ (6.40 g). Once the addition was complete, the mixture was refluxed for 4 h upon which the mixture was poured into ice water (500 mL) and extracted with CH₂Cl₂ (2×11 mL). The combined extracts were combined, dried over anhydrous MgSO₄, filtered, and then concentrated to afford the intermediate (10.6 g; 98%) as a light brown solid. ¹H-NMR (CDCl₃) δ=2.40 (s, 9 H), 2.70 (t, 2 H), 3.05 (t, 2 H), 7.50 (d, 1 H), 7.65 (d, 1 H).

Step F

To a cooled solution of (S)-2-methyl-CBS-oxazaborolidine (1M in toluene, 8.6 mL) and borane-methyl sulfide complex (1M in CH₂Cl₂, 43.0 mL) at −20° C. (internal temperature) in CH₂Cl₂ (200 mL) was added a solution of intermediate from Step E above (9.66 g, in 70 mL CH₂Cl₂) over a 10 h period via a syringe pump. After the addition was complete, the mixture was then quenched by the addition of MeOH (100 mL) at −20° C., warmed to room temperature and concentrated. The crude mixture was purified by flash chromatography (10% to 30% Et₂O/CH₂Cl₂ gradient) to afford the intermediate (8.7 g; 90%) as a colourless solid. ¹H-NMR (CDCl₃) δ=2.00 (m, 1 H), 2.35 (s, 3 H), 2.50 (m, 1 H), 2.90 (m, 1 H), 3.10 (m, 1 H), 5.25 (m, 1 H), 7.20 (d, 1 H), 7.50 (d, 1 H).

Step G

To a −78° C. cooled solution of intermediate from step F above (8.7 g) in CH₂Cl₂ (200 mL) under nitrogen was added triethylamine (15.9 mL) followed by methanesulfonyl chloride (4.5 mL). This mixture was stirred for 90 min and then NH₃ (˜150 mL) was condensed into the mixture using a dry ice/acetone cold finger at a rate of ˜3 mL/minute. After stirring at −78° C. for an additional 2 h, the mixture was gradually warmed to room temperature allowing the NH3 to evaporate from the reaction mixture. 1N NaOH (200 mL) was added and the aqueous layer was extracted with CH₂Cl₂ (2×100 mL). The combined extracts were dried over anhydrous MgSO₄, filtered, and then concentrated to afford crude material as a light brown oil. This oil was dissolved in Et₂O (200 mL) and hydrogen chloride (4M in dioxane, 10 mL) was added and the precipitate was collected and dried to give the intermediate (9.0 g; 90%). [M-NH₃Cl]⁺=209/211.

Step H

The intermediate from Step G above (5.2 g) was mixed in dry CH₂Cl₂ (50 mL) and cooled to 0° C. and to this cooled solution was added di-tert-butyl dicarbonate (5.0 g) followed by Et₃N (9.67 mL). After stirring for 3 h, the mixture was concentrated and redissolved in Et₂O (250 mL). This solution was washed with saturated NaHCO₃ (100 mL) and brine (100 mL). The organic layer was dried over anhydrous MgSO₄, filtered, and concentrated to afford the intermediate (7.28 g; 97%) as a colourless solid. ¹H-NMR (CDCl₃, free base) δ=1.80 (m, 1 H), 2.30 (s, 3 H), 2.60 (m, 1 H), 2.80 (m, 1 H), 2.90 (m, 1 H), 4.30 (t, 1 H), 7.00 (d, 1 H), 7.40 (m, H).

Step I

The intermediate from Step H above (7.2 g), zinc(II) cyanide (5.2 g) and Pd(PPh₃)₄ (2.6 g) were combined under nitrogen and anhydrous DMF (80 mL) was added. The yellow mixture was heated to 100° C. for 18 h and then concentrated under reduced pressure to afford crude material which was purified by flash chromatography (20% CH₂Cl₂/EtOAc) to give the intermediate (4.5 g; 75%) as an off-white solid. ¹H-NMR (CDCl₃) δ=1.50 (s, 3 H), 1.90 (m, 1 H), 2.40 (s, 3 H), 2.70 (m, 1 H), 2.80 (m, H), 2.95 (m, 1 H), 4.75 (m, 1 H), 5.15 (m, 1 H), 7.20 (d, 1 H), 7.50 (d, 1 H).

Step J

The intermediate from Step I above (1.0 g) was suspended in 6N hydrochloric acid (20 mL) and heated to 100° C. for 12 h upon which the solution become homogeneous. The solvent was removed under reduce pressure to give the intermediate (834 mg; quantitative) as a colourless solid. [M-NH₃Cl]⁺=175.

Step K

The intermediate from Step J above (1.0 g) was dissolved in anhydrous MeOH (20 mL) and cooled to 0° C. and anhydrous hydrogen chloride was bubbled through this solution for 2-3 min. The reaction mixture was then heated to reflux for 12 h. After cooling to room temperature, the solvent was removed under reduced pressure to give the title compound (880 mg; quantitative) as a colourless solid. [M-NH₃Cl]⁺=189.

### Preparative Example 2111

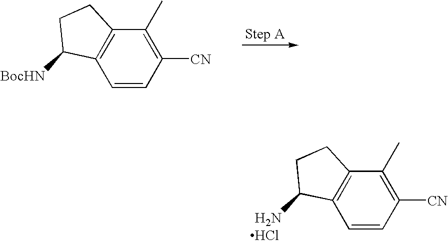 Step A

To the intermediate from the Preparative Example 2110, Step I above (108 mg) was added a solution of hydrogen chloride (4M in dioxane, 2 mL) and the resulting solution was allowed to stir at 22° C. for 6 h at which time a precipitate had formed. The mixture was concentrated to give the title compound (83 mg, >99%) as a colourless powder. [M-NH₃Cl]⁺=156.

### Preparative Example 2112

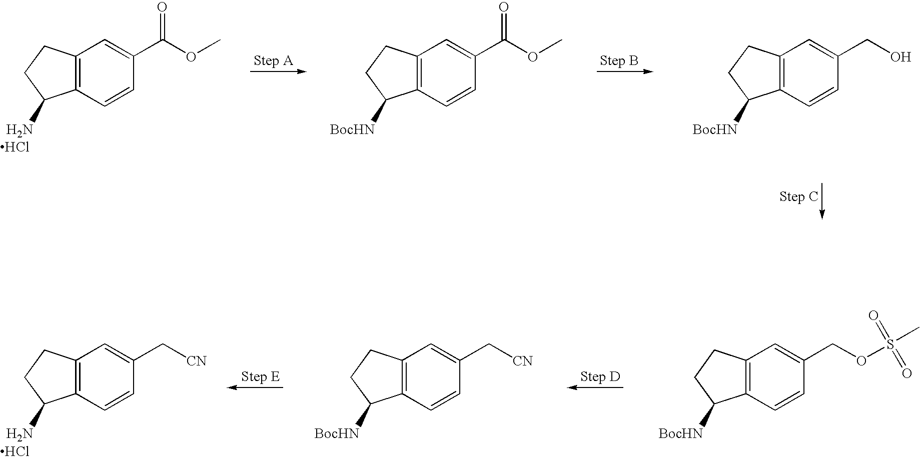 Step A

The hydrochloride salt of the intermediate from the Preparative Example 2105, Step B (450 mg) was dissolved in dichloromethane (30 mL). After addition of triethylamine (0.3 mL) and di-tert-butyl dicarbonate (480 mg), the reaction mixture was stirred at room temperature for 1.5 h. Diethylentriamine was added (1 mL) and the reaction mixture was washed with water and a solution of saturated ammonium chloride. The organic layer was dried (MgSO₄) and concentrated to afford the intermediate (560 mg; 96%), which was used without further purification for the next step. [MNa]⁺=314.

Step B

The intermediate from Step A above (560 mg) was dissolved in dry dichloromethane (10 mL) and cooled with an ice bath. A 1M solution of di-isobutyl aluminium hydride was added (10 mL) and the reaction mixture was allowed to warm up to room temperature. After stirring overnight the reaction was quenched with methanol (10 mL). Rochelle's salt was added and the mixture was stirred for another hour at room temperature. The mixture was extracted with ethyl acetate, the orgnaic layer was dried (MgSO₄) and concentrated to afford the intermediate (420 mg; 83%), which was used without further purification for the next step. [MNa]⁺=286.

Step C

The intermediate from Step B above (420 mg) was dissolved in dichloromethane (20 mL). After addition of triethylamine (450 μL) and methanesulfonyl chloride at 0° C., the reaction mixture was stirred for 3 h. Then the mixture was diluted with dichloromethane (20 mL) and washed with brine. The organic layer was dried (MgSO₄) and concentrated to afford the intermediate (560 mg; crude), which was used without further purification for the next step. [MNa]⁺=364.

Step D

The crude material from Step C above (560 mg) was dissolved in dimethylacetamide (20 mL). Sodium cyanide (400 mg) was added and the mixture was stirred overnight at 70° C. Ethyl acetate (80 mL) and brine (100 mL) were added. The organic layer was dried (MgSO₄) and concentrated. The remaining residue was purified by chromatography (silica, dichloromethane/acetone, 9:1) to afford the title compound (327 mg; 75% over two steps). [MNa]⁺=295.

Step E

A 4M solution of hydrochloric acid in dioxane (2 mL) was added to a suspension of the title compound from Step D above (110 mg) in dioxane (2 mL). The reaction mixture was stirred at room temperature overnight and was then concentrated to afford the title compound as the hydrochloric salt (90 mg; 99%). [M-NH₃Cl]⁺=156.

### Preparative Example 2113

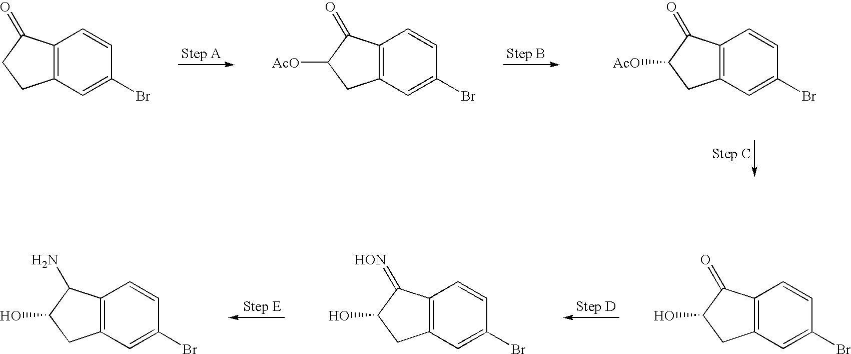 Step A

Commercially available 5-bromo-2,3-dihydroinden-1-one (2.10 g) and Mn(OAc)₃ dihydrate (9.0 g) were added to toluene (100 mL) and acetic acid (10 mL). The mixture was heated to reflux under a Dean-Stark condenser for 1.5 h. The mixture was diluted with diethyl ether and washed with brine twice. The organic was concentrated to afford the racemic intermediate (2.63 g; 98%) as a yellow solid. [MH]⁺=269/271.

Step B

The racemic intermediate from Step A above (2.63 g) and PS Amano (1 g) were added to acetonitrile (20 mL) and a PBS buffer solution (200 mL, pH 7). The hydrolysis reaction was monitored by LC/MS. After 1.5 h the mixture was extracted with diethyl ether twice. The combined organic layers were washed with brine, dried over MgSO₄, concentrated and purified by column chromatography (silica, hexanes/EtOAc) to afford the (S)-enantiomer (0.84 g; 32%) as a yellow solid. [MH]⁺=269/271.

Step C

The intermediate from Step B above (179 mg) and Sc(OTf)₃ (65 mg) were added to methanol (16 mL) and water (4 mL). The mixture was stirred at room temperature for 2 days and extracted with CH₂Cl₂ twice. The combined organic layers were concentrated and purified by column chromatography (silica, hexanes/EtOAc) to afford the intermediate (124 mg; 83%) as a yellow solid. [MH]⁺=227/229.

Step D

The intermediate from Step C above (124 mg), hydroxylamine hydrocholoride (42 mg) and sodium acetate (50 mg) were added to methanol (3 mL) and stirred at room temperature. After 15 h the mixture was diluted with H₂O and filtered. The solid collected was purified by column chromatography (silica, hexanes/EtOAc) to afford the intermediate (117 mg; two isomers in ratio of 1/1) as a colourless solid. [MH]⁺=242/244.

Step E

The compound from Step D above (103 mg) was dissolved in anhydrous diethyl ether (2 mL). The solution was cooled to −78° C. and lithium aluminum hydride (1M in Et₂O, 1.28 mL) was added dropwise. The mixture was heated to reflux and stirred for 15 h and then cooled down to −30° C. Water (0.5 mL) and 1M aqueous sodium hydroxide solution (0.5 mL) were added to the mixture slowly. The reaction mixture was warmed up to room temperature and filtered through Celite®. The filtrate was concentrated to afford the title compound (62 mg) as a solid. [MH]⁺=228/230.

### Preparative Example 2114

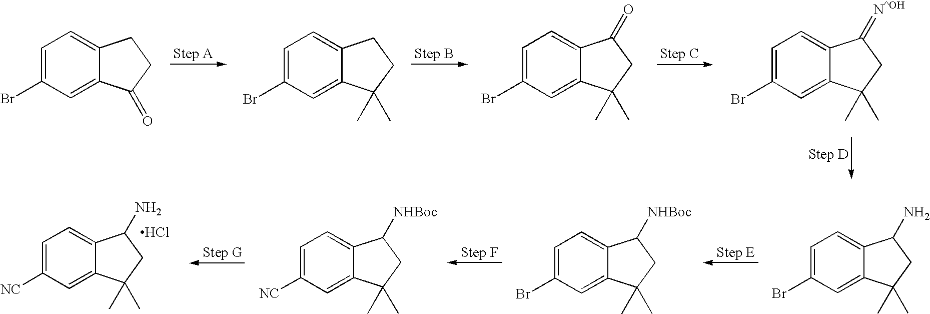 Step A

To a solution of TiCl₄ (3.54 g) in dichloromethane (20 ml) was added dimethyl zinc (1.3M in toluene, 15.5 mL) at −78° C. After 10 min at this temperature, commercially available 6-bromo-indan-1-one (3.58 g), dissolved in dichloromethane (20 mL) was added. After 2 h at −78° C. to −10° C. the mixture was poured onto ice and the aqueous layer was extracted with diethyl ether. The organic layer was dried (MgSO₄), concentrated and purified by column chromatography (silica, cyclohexane/EtOAc, 9:1) to afford the title compound (2.04 g; 53%) as a yellow oil. ¹H-NMR (CDCl₃) δ=1.25 (s, 6 H), 1.94 (t, 2 H), 2.82 (t, 2 H), 7.05 (d, 1 H), 7.20-7.30 (m, 3 H).

Step B

To a solution of the title compound from Step A above (2.10 g) in acetic acid was added a solution of CrO₃ (3.72 g) in 50% aqueous acetic acid (20 mL) at 55° C. and the mixture was stirred for 30 min at this temperature. After cooling to 0° C. 2-propanol (5 mL) was added and the mixture was diluted with ethyl acetate (400 mL), washed with 0.5M sodium hydroxide and brine, dried (MgSO₄), concentrated and purified by column chromatography (silica, cyclohexane/EtOAc, 9:1) to afford the title compound (829 mg; 37%) as an oil. [MH]⁺=239/241.

Step C

A mixture of the title compound from Step B above (829 mg), hydroxylamine hydrochloride (963 mg) and sodium hydrogencarbonate (1.17 g) in methanol (5 mL) was stirred at 60° C. for 16 h. Then the mixture was concentrated and the residue diluted with ethyl acetate. The organic layer was washed with water and brine, dried (MgSO₄) and concentrated to afford the title compound (898 mg; quantitative) as a foam. [MH]⁺=254/256.

Step D

To a solution of the title compound from Step C above (898 mg) in diethyl ether (10 mL) was added lithium aluminium hydride (1M in diethyl ether, 17.7 mL) at −78° C. The resulting mixture was warmed up to room temperature and then refluxed for 5 h. After this the mixture was cooled down to 0° C. and quenched with water (0.80 mL), 15% aqueous NaOH (2.4 mL) and water (2.4 mL), diluted with chloroform and filtered through Celite®. The organic layer was dried (MgSO₄) and concentrated to afford the title compound (687 mg) as an oil which was used without further purification. [MH]⁺=240/242.

Step E

A solution of the title compound from Step D above (687 mg), (Boc)₂O (812 mg) and triethylamine (376 μL) in tetrahydrofurane (10 mL) was stirred at room temperature for 16 h. Then the mixture was concentrated and purified by column chromatography (silica, cyclohexane/EtOAc, 9:1) to afford the title compound (927 mg; 77% over two steps) as a colourless oil. ¹H-NMR (CDCl₃) δ=1.20 (s, 3 H), 1.34 (s, 3 H) 1.48 (s, 9 H),1.76 (dd, 2 H), 2.45 (dd, 1 H), 4.70 (br d, 1 H), 5.20 (m, 1 H), 7.15 (d, 1 H), 7.22-7.35 (m, 2 H).

Step F

A mixture of the title compound from Step E above (927 mg), Zn(CN)₂ (192 mg) and Pd(PPh₃)₄ (157 mg) in DMF (50 mL) was stirred at 100° C. for 16 h. The mixture was concentrated and purified by column chromatography (silica, cyclohexane/EtOAc, 9:1) to afford the title compound (927 mg; 95%) as a colourless solid. [MH]⁺=287.

Step G

A solution of the title compound from Step F above (288 mg) in 4M HCl in dioxane (4 mL) was stirred at room temperature for 2 h. The mixture was concentrated to afford the title compound (220 mg; quantitative). [M-Cl]⁺=187.

### Preparative Example 2115

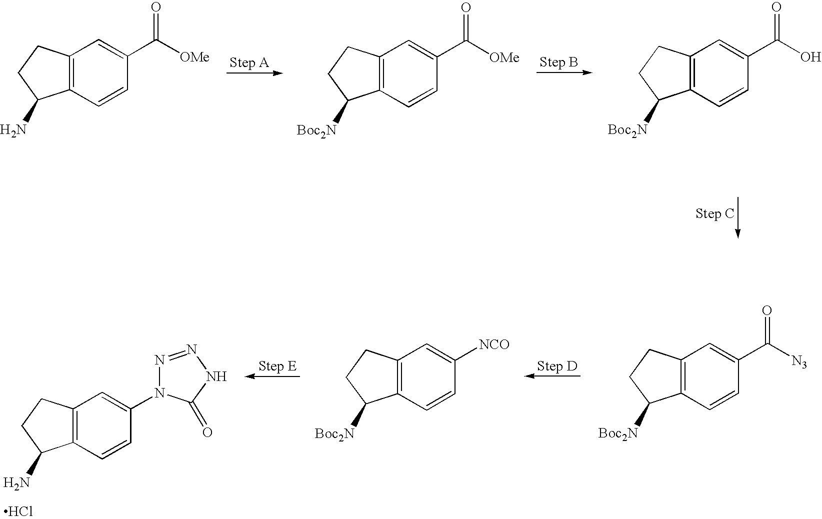 Step A

The intermediate from the Preparative Example 2105, Step B was treated with excess of di-tert-butylcarbonate and catalytic amounts of 4-dimethylaminopyridine in acetonitrile overnight. The volatiles were removed under reduced pressure and the residue dissolved in ethyl acetate, washed with brine, dried and evaporated. Purification by silica gel chromatography (hexanes/ethyl acetate) afforded the intermediate as a colourless solid.

Step B

The intermediate from Step A above (954 mg) was dissolved in a mixture of tetrahydrofuran (10 mL), methanol (5 mL) and water (5 mL). Sodium hydroxide (1M, 5 mL) was added dropwise. The reaction was stirred overnight. The volatiles were removed under reduced pressure and the residue dissolved in ethyl acetate, washed with an aqueous ammonium chloride solution, dried and evaporated to afford the intermediate (789 mg; 86%), which was used without further purification.

Step C

To the intermediate from Step B above (351 mg) in THF (2 mL) was added N-methylmorpholine (0.33 mL) and chloroisobutylformate (0.16 mL) at −10° C. The reaction was kept at the same temperature for 1 h. Diethyl ether (20 mL) and a saturated solution of sodium bicarbonate (5 mL) was added. The aqueous layer was separated and extracted with diethyl ether (10 mL). The combined organic layer was washed with brine, dried over magnium sulfate and concentrated to give the intermediate.

Step D

The intermediate from Step C above was dissolved in toluene (10 mL) and heated to reflux for 5 h until MS showed no starting material left (detection of the corresponding amine). The reaction mixture was concentrated to give intermediate, which was used crude in the next step.

Step E

To the intermediate from Step D above was added neat azidotrimethylsilane (1 mL) and heated to reflux overnight. The mixture was concentrated to dryness. To the remaining solid was added hydrogen chloride (4M in dioxane, 5 mL) and stirred for 1 h. Diethyl ether (10 mL) was added and the precipitate was filtered and washed with diethyl ether (10 mL) to give the title compound (230 mg, quantitative over three steps) as a colourless solid. [M-Cl]⁺=218.

### Preparative Example 2116

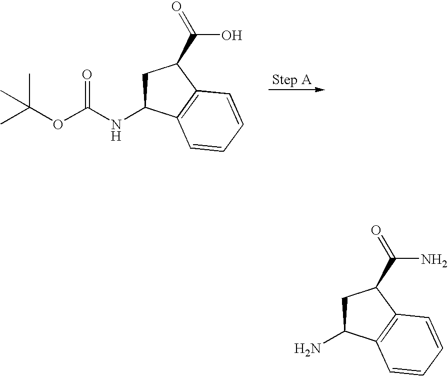 Step A

To commercially available 3-tert-butoxycarbonylamino-indan-1-carboxylic acid (0.5 g) in dry methylene chloride (6 mL) at -20° C. was added oxalyl chloride (0.17 mL) followed by N,N-dimethylformamide (0.2 mL) and the mixture was stirred for 1 h at −20° C., then 2 h at room temperature. The reaction was then concentrated to an oil. The oil was dissolved in tetrahydrofurane (2 mL) and then slowly added to condensed ammonia (approx. 4 mL) at approx. −40° C. The reaction mixture was stirred at approx. −30° C. for 1 h and then allowed to slowly warm to room temperature (˜10 h). The volatile components of the reaction mixture were removed under reduced pressure to give the title compound (0.15 g; 48%) as a tan solid. [MH]⁺=177.

### Preparative Example 2117

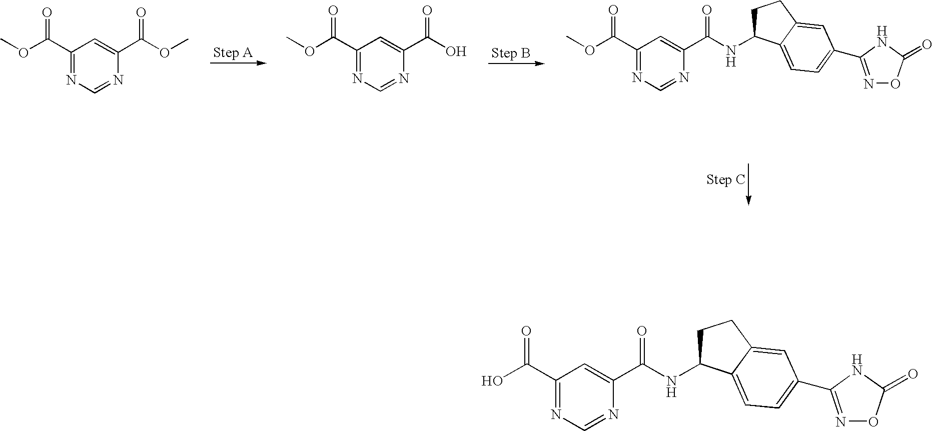 Step A

To a solution of sodium hydroxide (1.00 g) in dry methanol (50 mL) was added commercially available pyrimidine-4,6-dicarboxylic acid dimethyl ester (4.91 g). The resulting suspension was stirred at room temperature for 1 h. Then a 4M solution of hydrochloric acid in dioxane (6.25 mL) was added and stirring at room temperature was continued for 10 min. The mixture was concentrated and purified by flash chromatography (silica, dichloromethane/methanol) to afford the title compound (3.48 g; 76%). [MH]⁺=183.

Step B

To a suspension of the title compound from Step A above (492 mg) in dry tetrahydrofurane (54 mL) was added N-methylmorpholine (720 mL). The resulting mixture was placed in a acetone/dry ice bath (−30° C.). At this temperature, ethyl chloroformate (265 μL) was added and stirring was continued for 1 h while keeping the temperature of the acetone/dry ice bath below −25° C. Then the title compound from Preparative Example 2106, Step C was added and stirring was continued for 16 h while the acetone/dry ice bath was allowed to warm to ˜15° C. The mixture was concentrated and purified by flash chromatography (silica, dichloromethane/methanol) to give a slightly yellow solid. This material was washed with dichloromethane (2×20 mL) to afford the title compound as a colourless solid (703 mg; 68%).

[MH]⁺=382.

Step C

The title compound from Step B above (552 mg) was dissolved in a 0.5M solution of sodium hydroxide in dry methanol (6.2 mL). The reaction mixture was stirred at room temperature for 1 h and then concentrated to afford a beige solid. This material was dissolved in water (6.2 mL) and treated with a 1M aqueous solution of hydrochloric acid (6.2 mL). The resulting suspension was ultrasonificated for 2 h and then filtered. The remaining solid was washed with water (2×6.2 mL), dissolved in methanol (62 mL), concentrated and dried under reduced pressure for 24 h to afford the title compound (483 mg; 89%) as a colourless solid. [MH]⁺=368.

### Preparative Example 2118

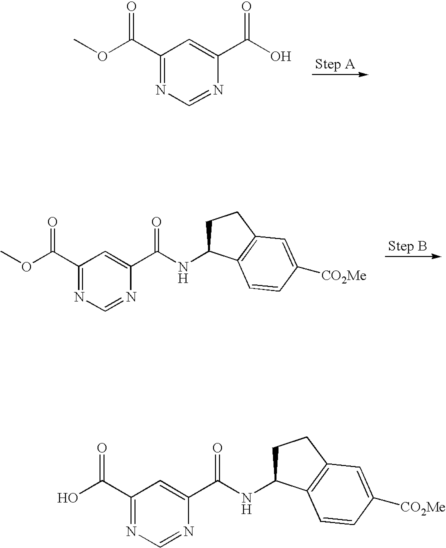 Step A

To a solution of the title compound from the Preparative Example 2117, Step A (607 mg) and N-methylmorpholine (370 mg) in THF (40 mL) was added ethyl chloroformate (361 mg) at −30° C. After 1.5 h at this temperature a suspension of the title compound from the Preparative Example 2105, Step B (759 mg) and N-methylmorpholine (438 mg) in THF (20 mL) was added and the resulting mixture was stirred for 16 h at −30° C. to room temperature. The mixture was concentrated and the residue diluted with ethyl acetate. The organic layer was washed with water and brine, dried (MgSO₄) and concentrated to afford the title compound (970 mg; 82%) as an off-white foam. [MH]⁺=356.

Step B

To a solution of the title compound of Step A above (920 mg) in methanol (15 mL) was added a sodium hydroxide (0.5M in methanol, 6.25 mL) of at room temperature. After 1 h at room temperature the mixture was diluted with 1M hydrochloric acid. The aqueous layer was extracted with ethyl acetate and the combined organic layers were dried (MgSO₄), concentrated and purified by column chromatography (silica, chloroform/MeOH 85:15) to afford the title compound (743 mg; 83%) as a colourless solid. [MH]⁺=342.

### Preparative Example 2119

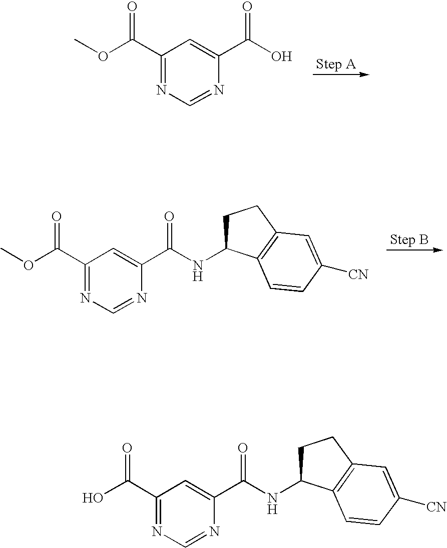 Step A

A solution of the title compound from the Preparative Example 2117, Step A (174 mg), the title compound from the Preparative Example 2104, Step F (169 mg), PyBroP (470 mg) and N-methylmorpholine (240 μL) in dry DMF (8 mL) was stirred at room temperature overnight. The mixture was concentrated and the residue diluted with ethyl acetate, washed with 10% citric acid, saturated sodium hydrogencarbonate solution and brine, dried (MgSO₄) and concentrated. Purification by column chromatography (silica, cyclohexane/ethyl acetate, 6:4 to 4:6) afforded the title compound (203 mg; 73%) as a colourless foam. [MH]⁺=323.

Step B

To the title compound of Step A above (203 mg) was added a sodium hydroxide (0.5M in methanol, 1.3 mL) of at room temperature. After 5 h at room temperature the mixture was evaporated and diluted with 1M hydrochloric acid (0.7 mL). The precipitate was filtered to afford the title compound (157 mg; 81%) as a colourless solid. [MH]⁺=309.

### Preparative Example 2120

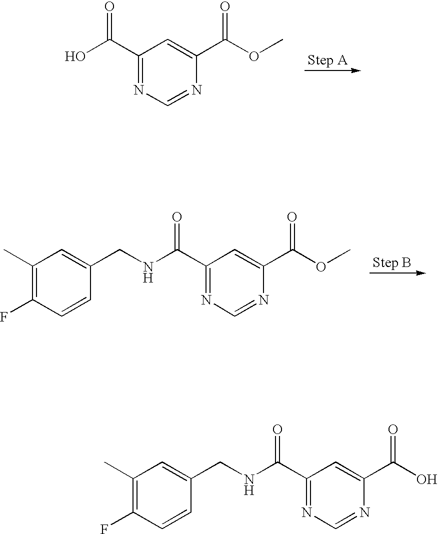 Step A

To a solution of the title compound from the Preparative Example 2117, Step A (2.29 g) and N-methylmorpholine (3.32 mL) in dry THF (250 mL) was added ethyl chloroformate (1.19 mL) at −30° C. After 1 h at this temperature 4-fluoro-3-methylbenzylamine (1.75 g) was added and the resulting mixture was stirred for 16 h allowing the temperature to raise from −30° C. to 10° C. The mixture was concentrated and absorbed on silica. Purification by column chromatography (silica, cyclohexane/ethyl acetate) afforded the title compound (2.39 g; 62%) as a colourless solid. [MH]⁺=304.

Step B

To a solution of the title compound of Step A above (2.39 g) in tetrahydrofurane (50 mL) and water (50 mL) was added a lithium hydroxide (496 mg) at room temperature. After 2 h at room temperature the mixture was acidified with 1M hydrochloric acid to pH 2. The aqueous layer was extracted with ethyl acetate twice and the combined organic layers were dried (MgSO₄) and concentrated to afford the title compound (2.23 g; 97%) as a colourless solid.

[MH]⁺=290.

### Preparative Example 2121

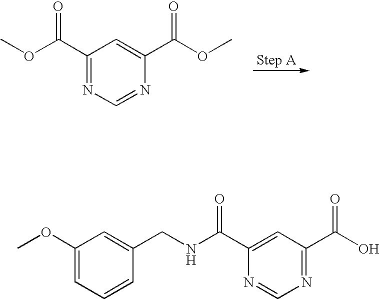 Step A

A solution of commercially available pyrimidine-4,6-dicarboxylic acid dimethyl ester (1.96 g) and commercially available 3-methoxy-benzylamine (1.38 mL) in dry N,N-dimethylformamide (10 mL) was placed in a preheated oil bath (˜80° C.). After stirring at this temperature for 18 h the mixture was concentrated and flash filtered (silica, cyclohexane/ethyl acetate). The obtained material was suspended in dry tetrahydrofurane (10 mL) and treated with a solution of lithium hydroxide (642 mg) in water (15 mL). The resulting mixture was stirred at room temperature for 16½ h, diluted with water (35 mL), washed with dichloromethane (3×50 mL) and acidified by addition of a 1M aqueous solution of hydrochloric acid (20 mL). The formed precipitate was isolated by suction, washed with water (2×50 mL) again suspended/dissolved in water (200 mL) and ultrasonificated for 5 min. The remaining precipitate was isolated by suction and dried under reduced pressure to afford the title compound (700 mg; 24%). [MH]⁺=288.

### Preparative Example 2122

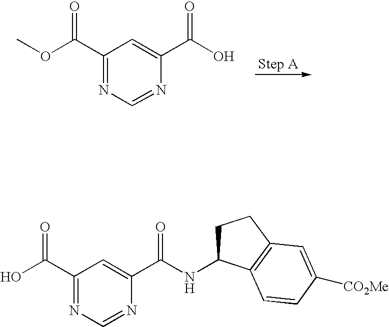 Step A

Following a similar procedure as that described in the Preparative Example 2118, except using the title compound from the Preparative Example 2117, Step A and the title compound from the Preparative Example 2110, Step K the intermediate was obtained in 38% yield. [MH]⁺=356.

### Preparative Example 2123

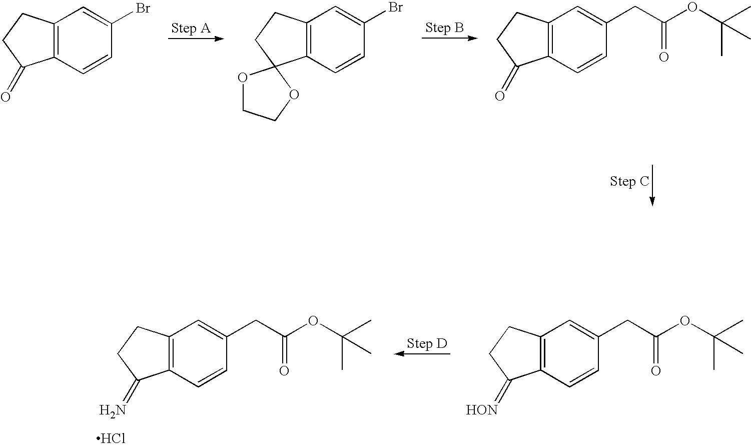 Step A

A mixture of 5-bromoindanone (3.04 g), ethylene glycol (10 mL) and toluolsulfonic acid (200 mg) in dry toluene (80 mL) was refluxed with a Dean-Stark for 8 h. After cooling was added potassium carbonate and the mixture absorbed on silica. Purification by flash chromatography (silica, cyclohexane/ethyl acetate 95:5) afforded the title compound (1.41 g; 38%). [MH]⁺=254/256.

Step B

To a solution of the title compound from Step A above (1.44 g), bis(dibenzylideneacetone)palladium (326 mg) and tri-tert-butylphosphine (0.1M in dry toluene, 5.6 mL) was added a solution of tert-butyl acetate (840 μL) and lithium dicyclohexylamide (1.38 g) in dry toluene (5 mL) under argon. The mixture was stirred overnight, diluted with ethyl acetate and washed with 10% citric acid, a saturated solution of sodium hydrogen carbonate and brine, dried, evaporated and purified by column chromatography (silica, cyclohexane/EtOAc, 9:1 to 8:2) to afford an oil, which was dissolved in acetone (45 mL) and water (5 mL). After adding pyridinium p-toluenesulphonate (120 mg), the mixture was refluxed for 2 h, concentrated, diluted with ethyl acetate and washed with a saturated solution of sodium hydrogen carbonate and brine, dried, evaporated and purified by column chromatography (silica, cyclohexane/EtOAc, 9:1 to 8:2) to afford the title compound (980 mg; 66%) as bright yellow crystals. [MH]⁺=247.

Step C

A mixture of the title compound from Step B above (891 mg), hydroxylamine hydrochloride (780 mg) and sodium acetate (780 mg) in dry methanol (20 mL) was refluxed for 1.5 h. The mixture was concentrated and the residue diluted with ethyl acetate. The organic layer was washed with water and brine, dried (MgSO₄) and concentrated to afford the title compound (980 mg; quantitative) as a bright yellow oil, which crystallized upon standing. [MH]⁺=262.

Step D

To the intermediate from Step C above (296 mg) was added zinc dust (500 mg) and 2N hydrochloric acid. The mixture was stirred overnight, basified with 1N sodium hydroxide extracted with chloroform. The organic layer was dried (MgSO₄) and concentrated to afford an oil, which was treated with hydrogen chloride (4N in dioxane, 400 μL), evaporated, slurried in diethyl ether and filtered to afford the intermediate (76 mg; 24%) as a colourless solid. [M-NH₃Cl]⁺=231.

### Preparative Example 2124

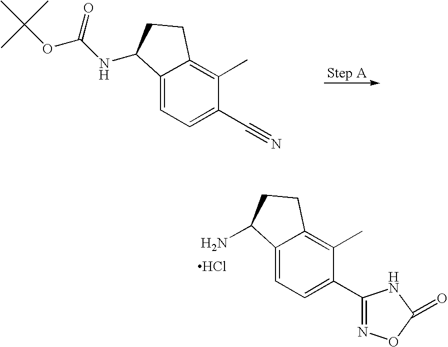 Step A

If one were to treat the intermediate from the Preparative Example 2110, Step I similar as described in the Preparative Example 2106; Step A to Step C, one would obtain the title compound.

### Preparative Example 2125

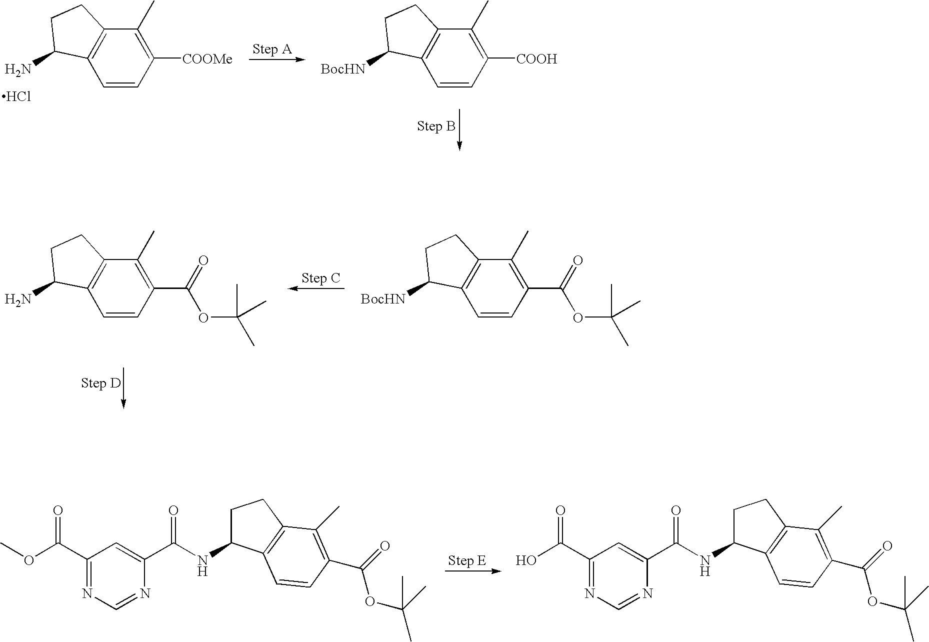 Step A

The intermediate from Preparative Example 2105, Step B (1.5 g) was mixed in dry CH₂Cl₂ (50 mL) and cooled to 0° C. and to this cooled solution was added di-tert-butyl dicarbonate (1.6 g) followed by Et₃N (1 mL). After stirring for 3 h, the mixture was concentrated and redissolved in Et₂O (250 mL). This solution was washed with saturated NaHCO₃ (100 mL) and brine (100 mL). The organic layer was dried over anhydrous MgSO₄, filtered, and concentrated to afford the intermediate (7.28 g; 97%) as a colourless solid which was dissolved in tedrahydrofurane (60 mL). To the mixture was added a 1M aqueous LiOH solution (60 mL) and the mixture was stirred at 50° C. for 2 h. The mixture was concentrated to dryness and redissolved in water, acidified to pH=5 with hydrochloric acid and extracted with ethyl acetate. The organic layer was dried (MgSO₄) and concentrated to afford the intermediate as colourless solid (1.87 g). [MNa]⁺=314.

Step B

To a solution of the title compound from Step A above (1.87 g) in dry toluene (15 mL) was added Di-tert-butoxymethyl dimethylamine (6.2 mL) at 80° C. At this temperature the mixture was stirred for 3 h. After cooling to room temperature the mixture was concentrated and purified by column chromatography (silica, dichloromethane) to afford the intermediate (820 mg; 38%) as a colourless solid. [MNa]⁺=370.

Step C

To a solution of the title compound from Step B above (820 mg) in tert-butyl acetate (40 mL) was added sulfuric acid (0.65 mL) at room temperature. The mixture was stirred for 5 h and concentrated to dryness. The residue was dissolved ethyl acetate and washed with a saturated solution of sodium hydrogen carbonate and brine. After drying (MgSO₄) the intermediate (640 mg; 99%) was obtained as a colourless solid. [M-NH₂]⁺=231.

Step D

To a solution of the title compound from Step C above (360 mg) in dry dimethylformamide (5 mL) was added bromotrispyrrolidinophosphonium hexafluorophosphate (1.1 g), the intermediate from the Preparative Example 2117, Step A (310 mg) and N-methylmorpholine (0.5 mL). The mixture was stirred at room temperature overnight and concentrated to dryness. The residue was dissolved in water and extracted with ethyl acetate. After drying (MgSO₄) the solution was concentrated and purified by chromatography (silica, cyclohexene/ethyl acetate) to afford the title compound as a colourless solid (285 mg; 48%).

[MNa]⁺=434.

Step E

The title compound from Step D above (285 mg) was dissolved in a 0.5M solution of sodium hydroxide in dry methanol (1.5 mL). The reaction mixture was stirred at room temperature for 2 h and then concentrated to afford a beige solid. This material was dissolved in water (6.2 mL) and treated with a 1M aqueous solution of hydrochloric acid (2 mL). The resulting suspension was diluted with water and extracted with ethyl acetate. After drying (MgSO₄) the solution was concentrated to afford the title compound (282 mg; quantitative) as a colourless solid. [MNa]⁺=420.

### Example 1

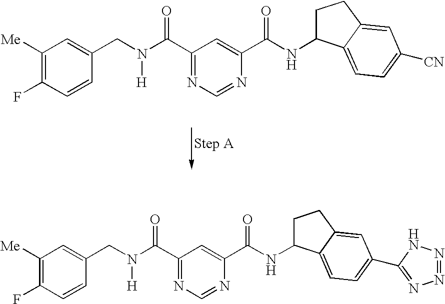 Step A

A mixture of pyrimidine-4,6-dicarboxylic acid 4-[(5-cyano-indan-1-yl)-amide]6-(4-fluoro-3-methyl-benzylamide) (75.8 mg) from Preparative Example 1, dibutyl tin oxide (9 mg), azidotrimethylsilane (47 μL), and toluene (1.5 mL) under an atmosphere of Ar in a sealed vial was allowed to stir at 110° C. for 18 h. The reaction mixture was concentrated and purified by silica gel chromatography (9:1 CH₂Cl₂: MeOH, R_{f}=0.2) to give an off-white solid (30 mg; 36%). [M-H⁺]³¹=471.6.

### Example 5

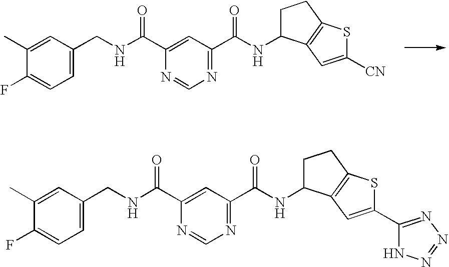

The corresponding carbonitrile (23.4 mg), Bu₂SnO (2.7 mg) and TMSN₃ (36 μL) were added to dioxane (1 mL). The mixture was heated up to 100° C. and stirred for 24 h. The solvent was evaporated in vaccuo. The residue was chromatographed on silica gel to afford 20.6 mg of white solid (80%). [MH]⁺=478.3.

### Example 8

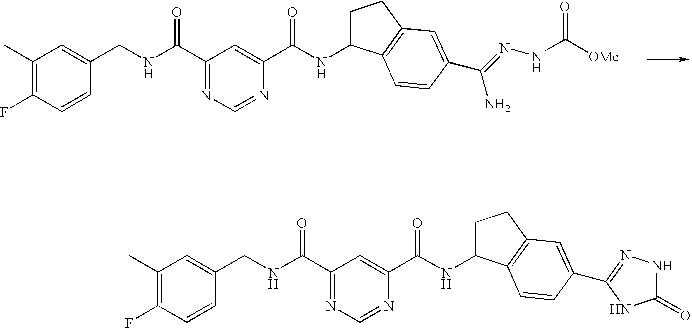

If one were to heat the title compound from Preparative Example 8, Step B dissolved in N-methyl pyrolidinone (5 mL) for 5 h, one would obtain the triazolone product.

### Example 9a

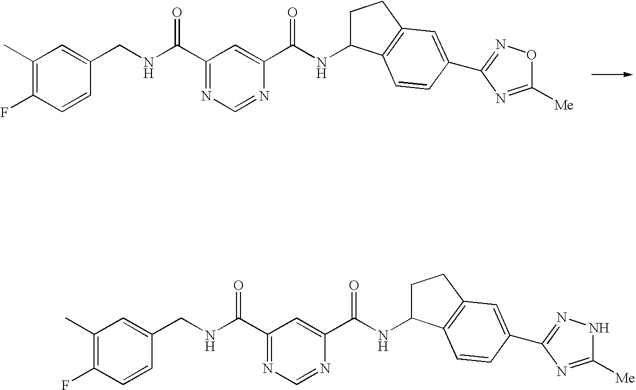

If one were to heat the title compound from Preparative Example 9a, Step C in hydrazine and methanol, one would obtain the desired triazole.

### Example 9b

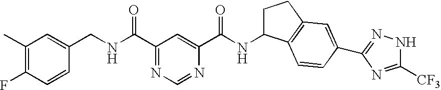

If one were to follow the procedures outlined in Preparative Example 9a and Example 9a but using instead trifluoroacetic anhydride instead of acetyl chloride in Preparative Example 9a, Step B, one would obtain the desired trifluoromethyltriazole.

### Examples 10-152

If one were to treat the nitrilles indicated in Table 4 below similar as described in the Example 1, one would obtain the tetrazoles indicated.

TABLE 4

| Ex. # | Starting Material | Product |
|---|---|---|
| 10 | 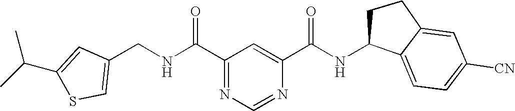 | 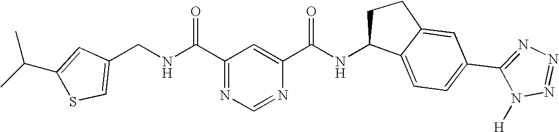 |
| 11 | 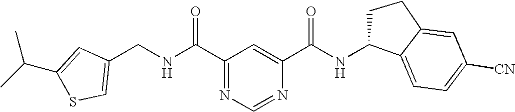 | 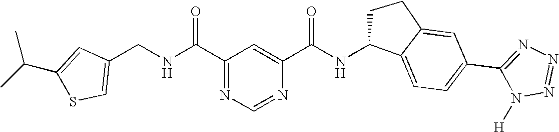 |
| 13 | 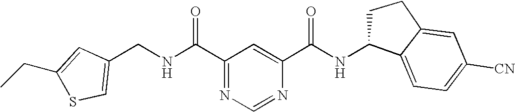 | 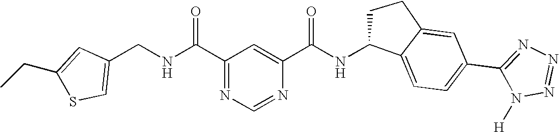 |
| 14 | 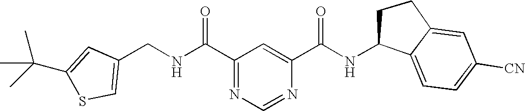 | 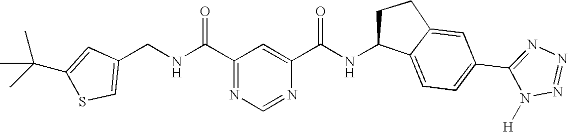 |
| 15 | 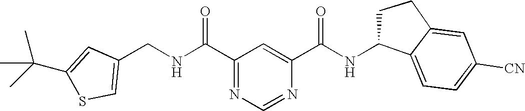 | 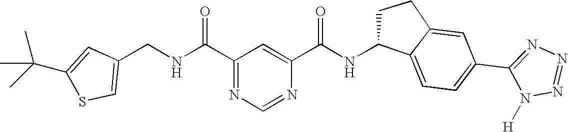 |
| 16 | 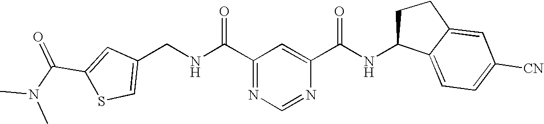 | 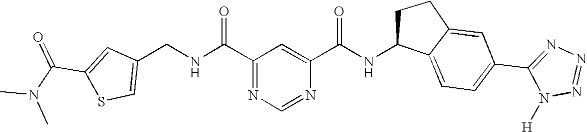 |
| 17 | 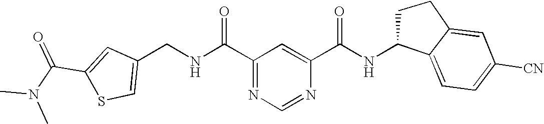 | 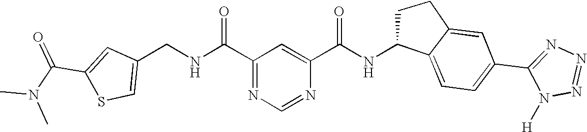 |
| 18 | 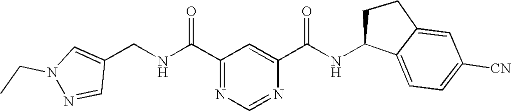 | 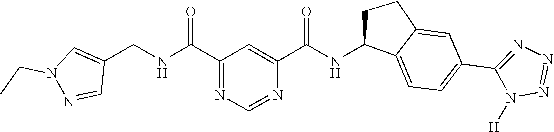 |
| 19 | 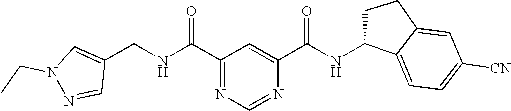 | 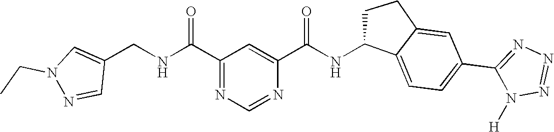 |
| 20 | 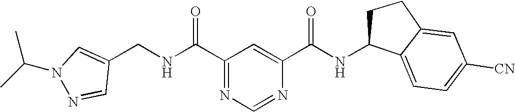 | 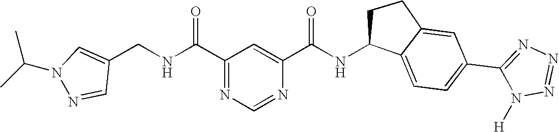 |
| 21 | 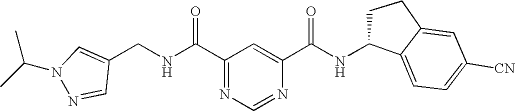 | 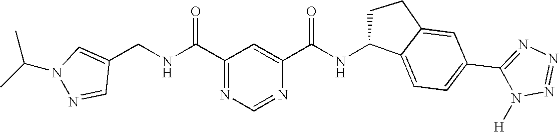 |
| 22 | 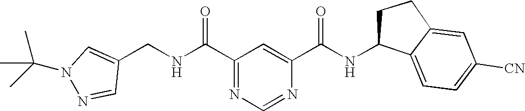 | 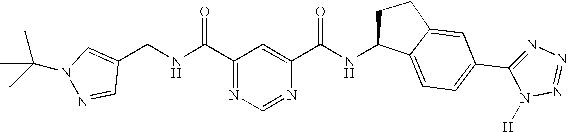 |
| 23 | 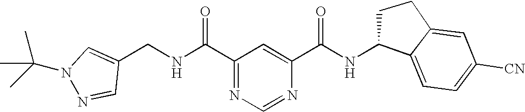 | 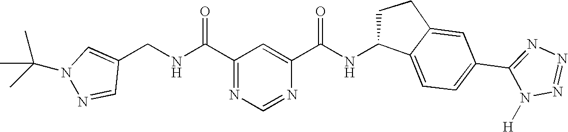 |
| 24 | 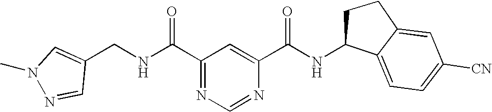 | 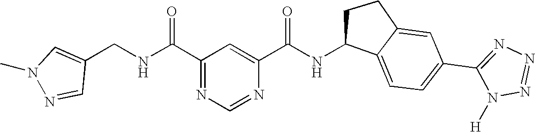 |
| 25 | 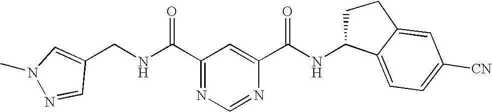 | 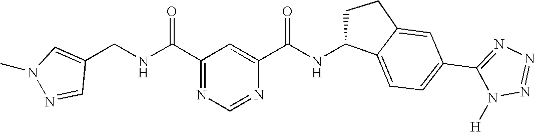 |
| 26 | 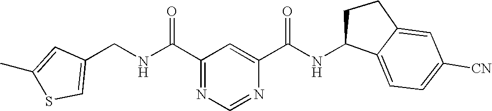 | 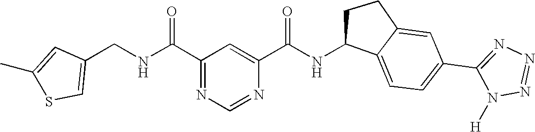 |
| 27 | 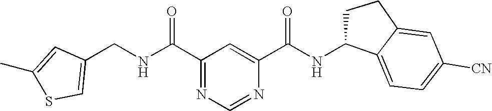 | 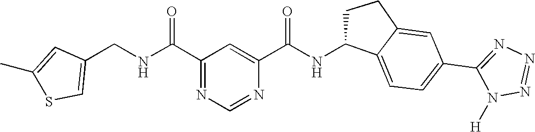 |
| 28 | 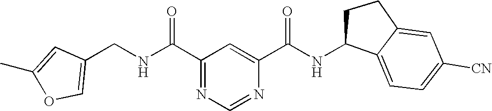 | 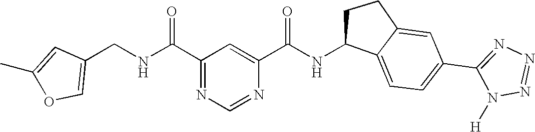 |
| 29 | 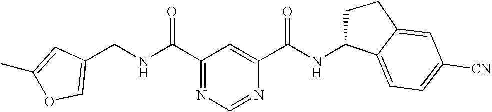 | 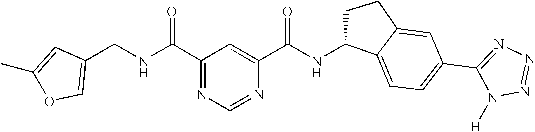 |
| 30 | 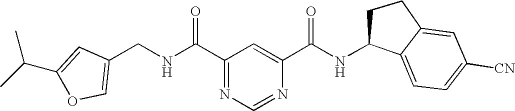 | 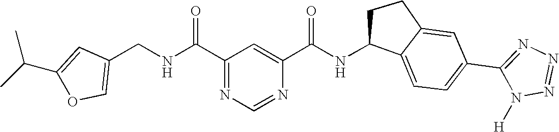 |
| 31 | 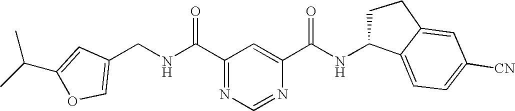 | 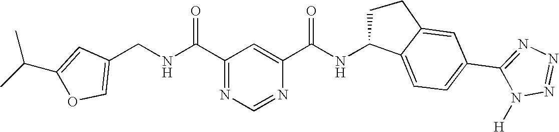 |
| 32 | 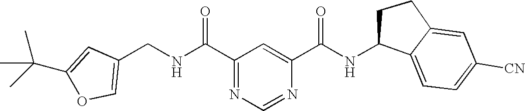 | 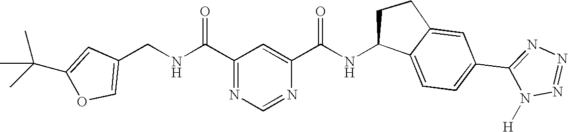 |
| 33 | 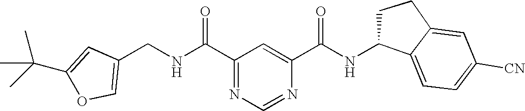 | 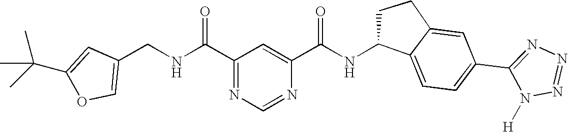 |
| 35 | 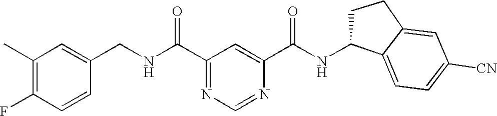 | 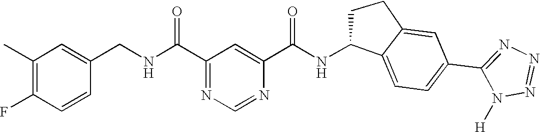 |
| 36 | 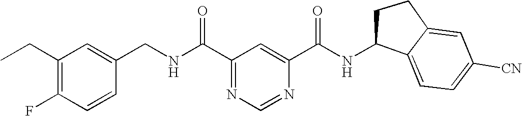 | 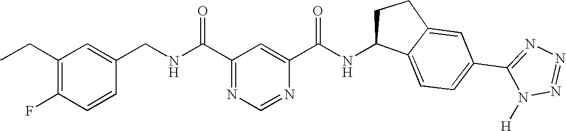 |
| 37 | 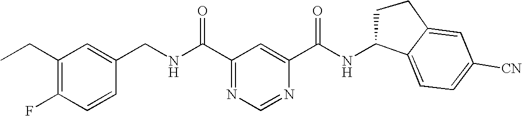 | 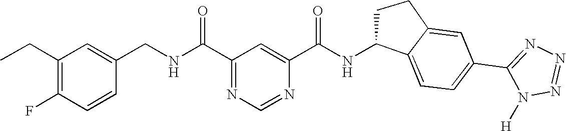 |
| 38 | 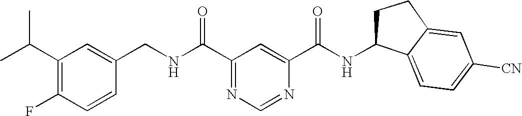 | 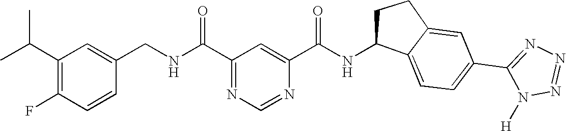 |
| 39 | 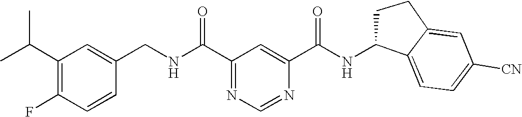 | 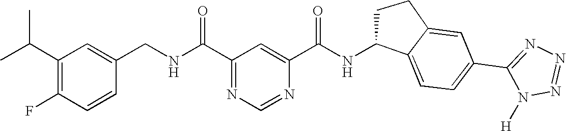 |
| 40 | 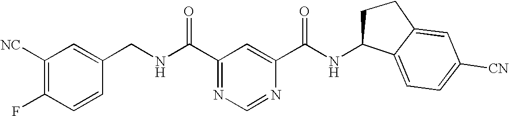 | 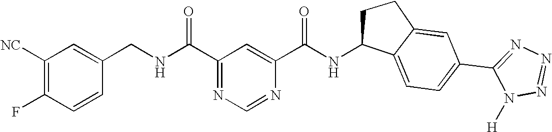 |
| 41 | 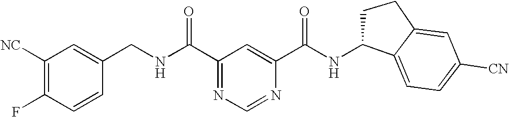 | 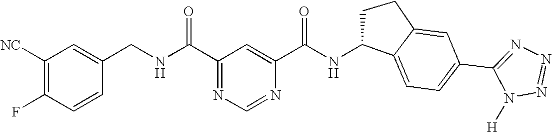 |
| 42 | 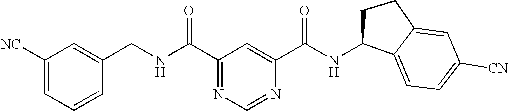 | 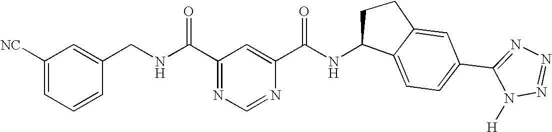 |
| 43 | 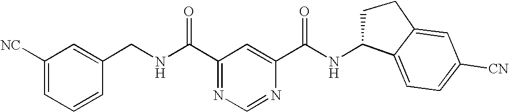 | 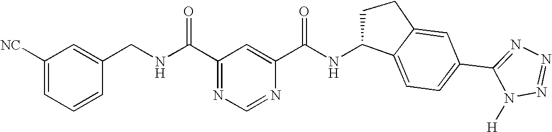 |
| 44 | 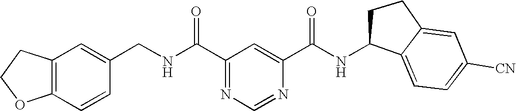 | 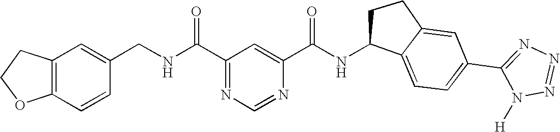 |
| 45 | 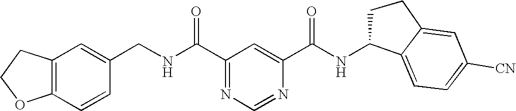 | 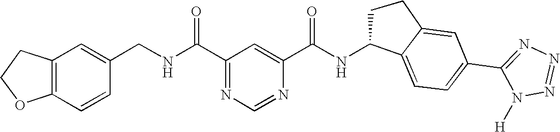 |
| 46 | 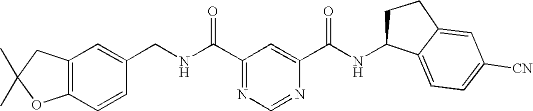 | 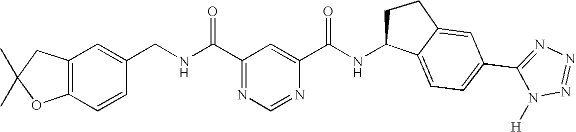 |
| 47 | 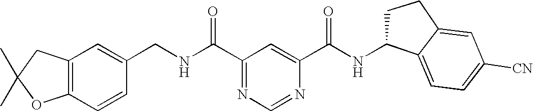 | 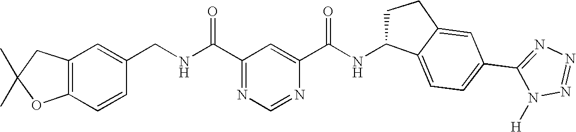 |
| 48 | 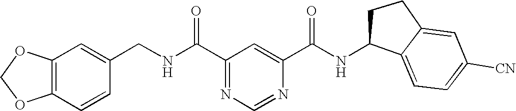 | 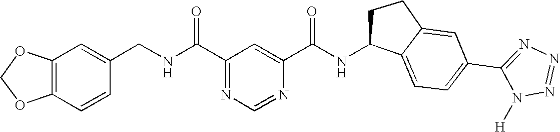 |
| 49 | 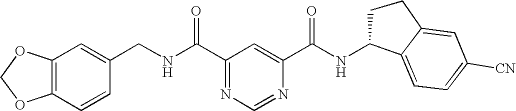 | 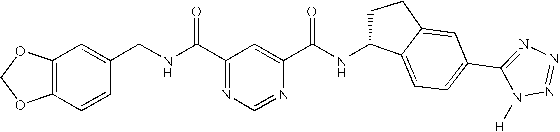 |
| 50 | 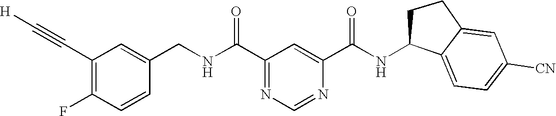 | 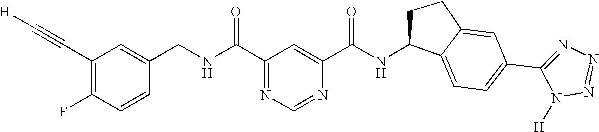 |
| 51 | 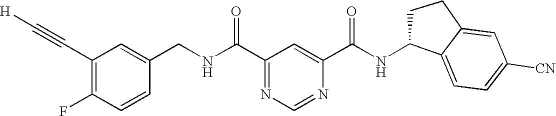 | 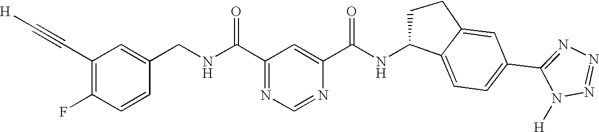 |
| 52 | 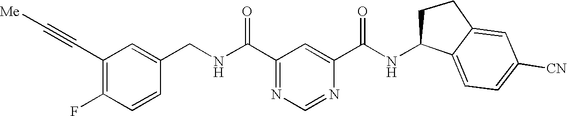 | 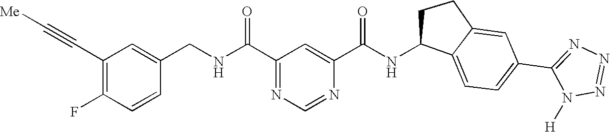 |
| 53 | 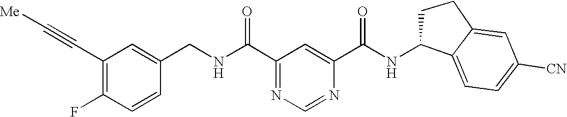 | 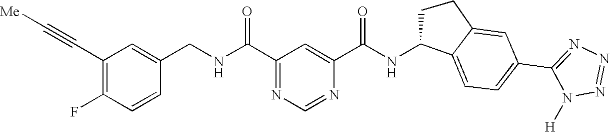 |
| 54 | 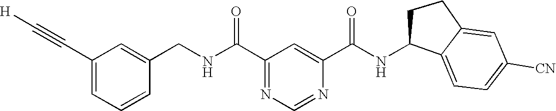 | 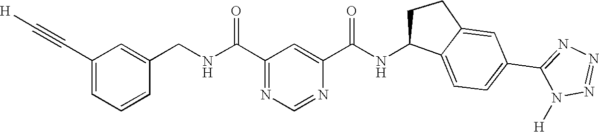 |
| 55 | 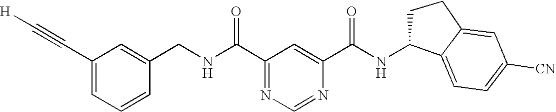 | 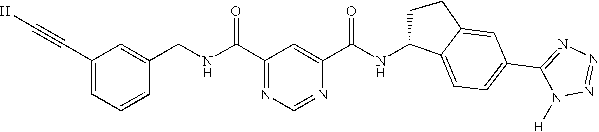 |
| 56 | 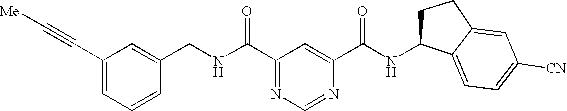 | 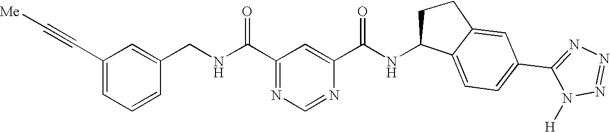 |
| 57 | 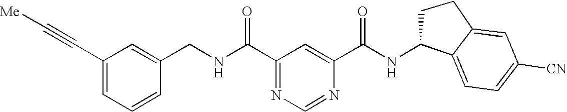 | 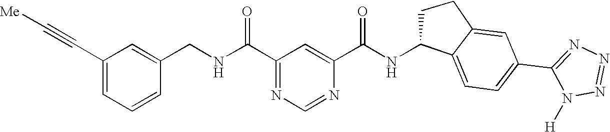 |
| 58 | 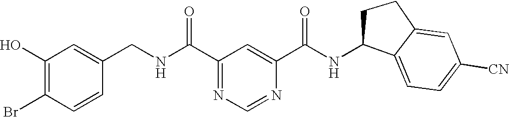 | 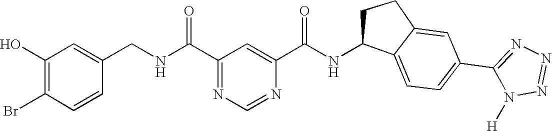 |
| 59 | 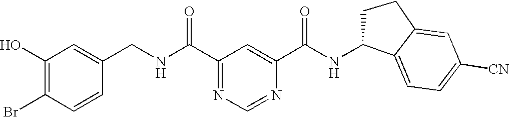 | 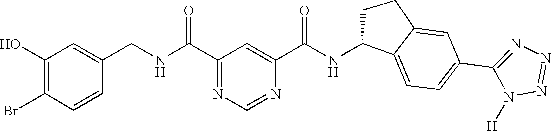 |
| 60 | 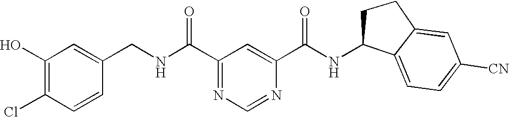 | 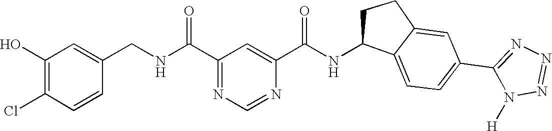 |
| 61 | 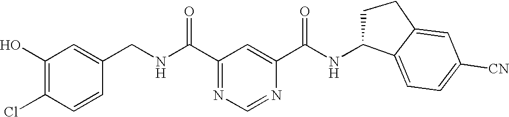 | 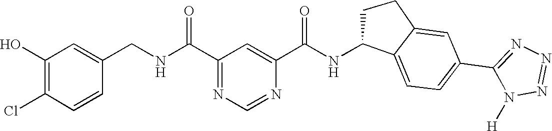 |
| 62 | 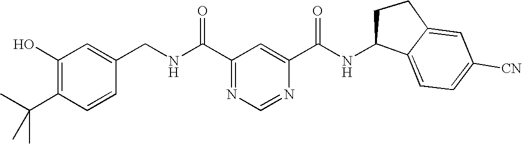 | 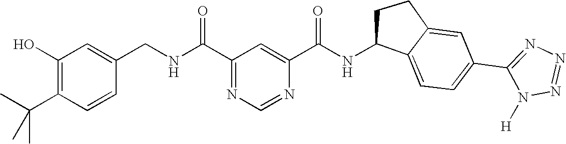 |
| 63 | 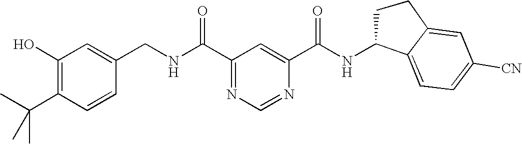 | 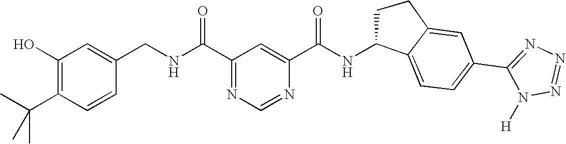 |
| 65 | 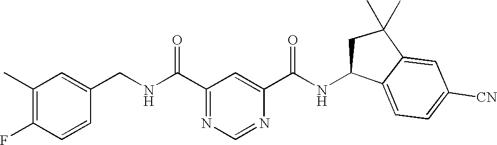 | 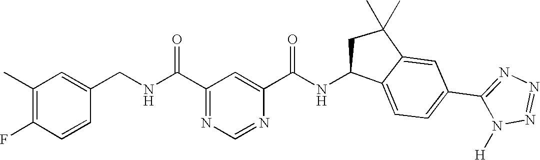 |
| 66 | 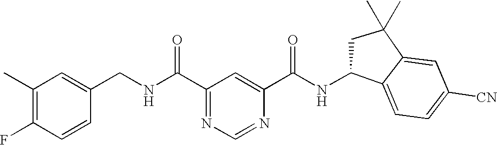 | 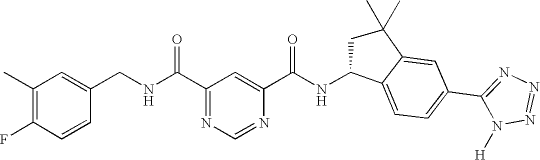 |
| 67 | 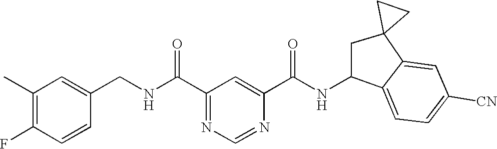 | 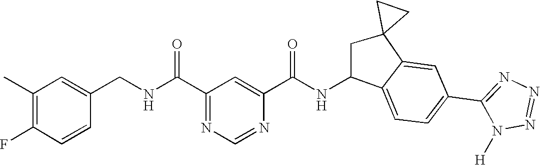 |
| 68 | 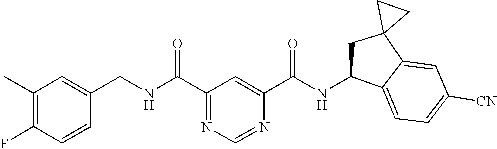 | 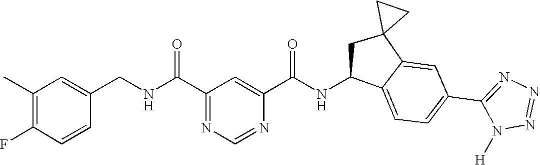 |
| 69 | 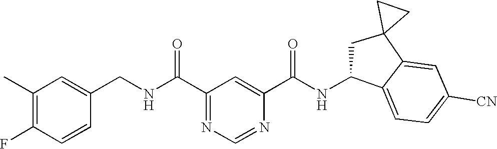 | 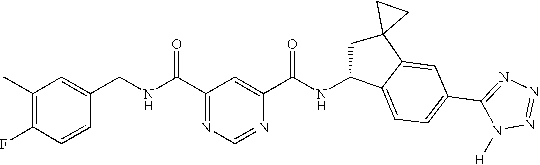 |
| 71 | 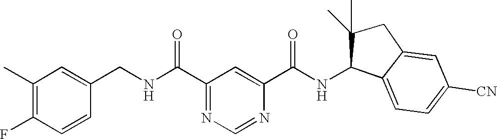 | 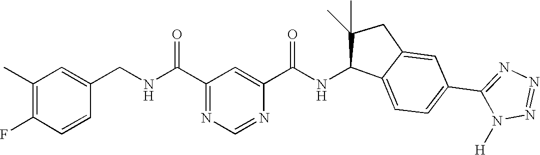 |
| 72 | 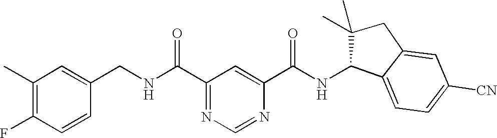 | 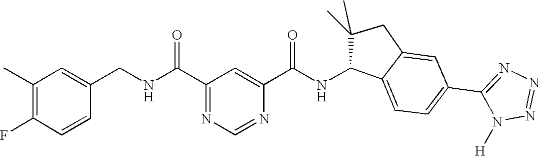 |
| 73 | 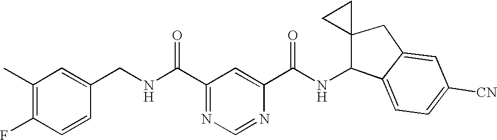 | 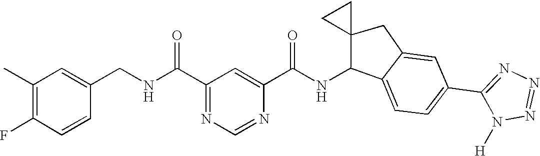 |
| 74 | 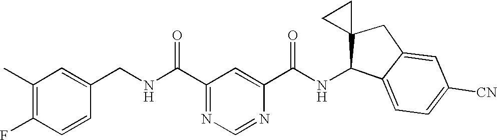 | 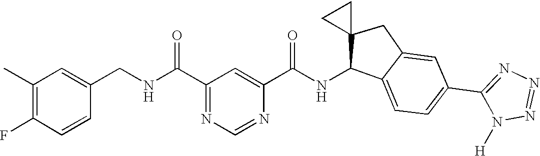 |
| 75 | 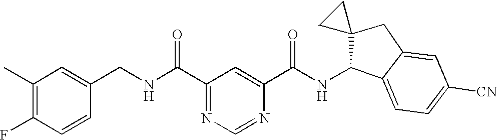 | 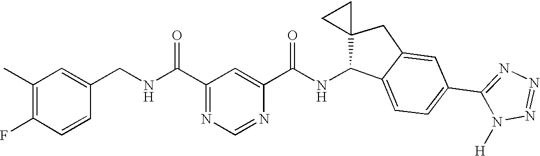 |
| 76 | 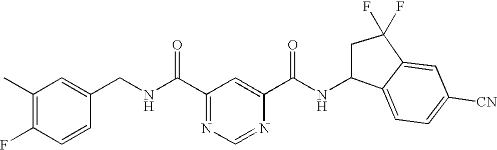 | 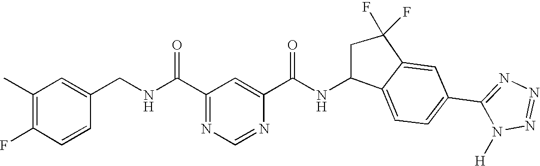 |
| 77 | 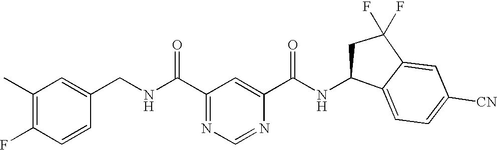 | 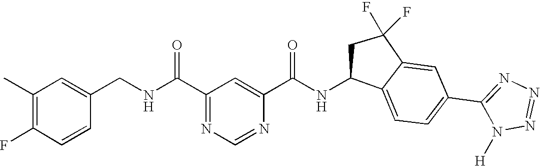 |
| 78 | 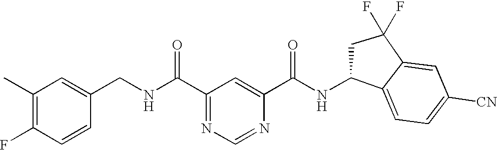 | 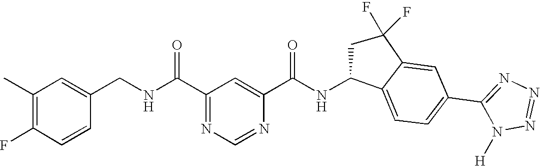 |
| 79 | 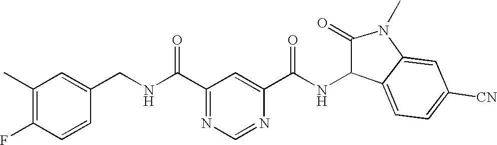 | 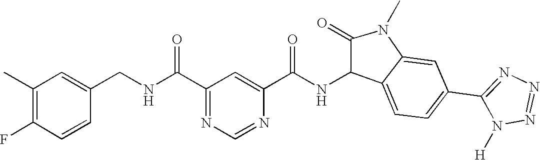 |
| 80 | 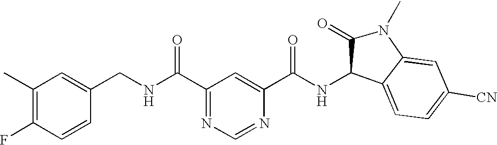 | 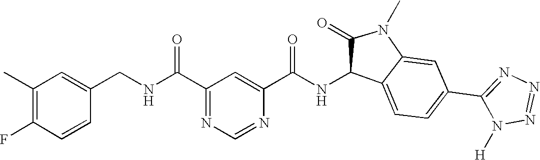 |
| 81 | 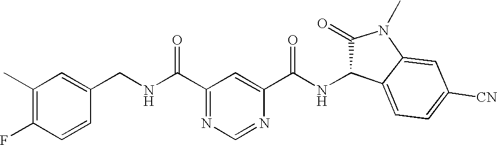 | 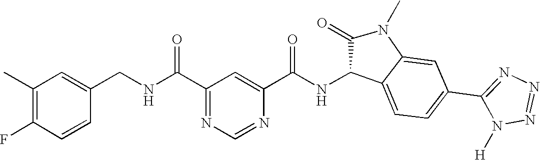 |
| 82 | 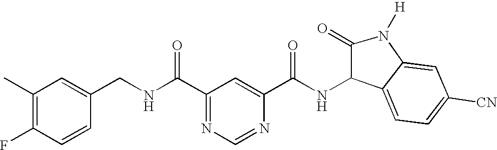 | 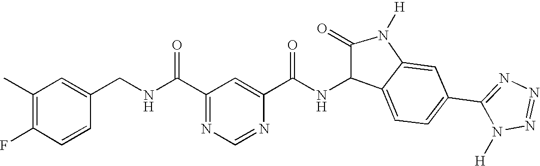 |
| 83 | 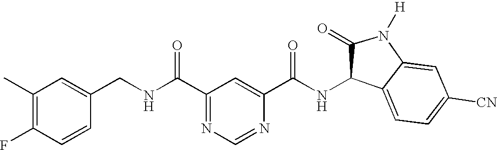 | 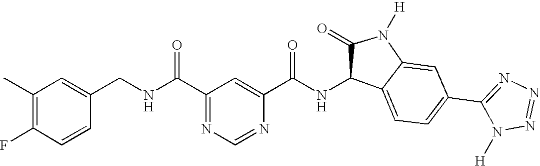 |
| 84 | 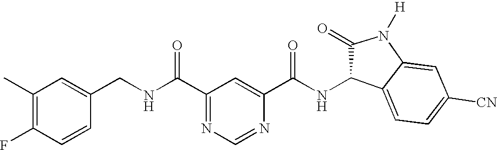 | 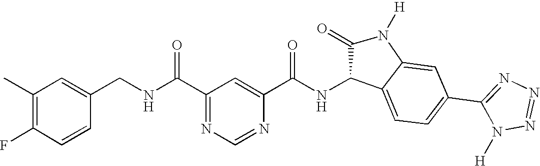 |
| 85 | 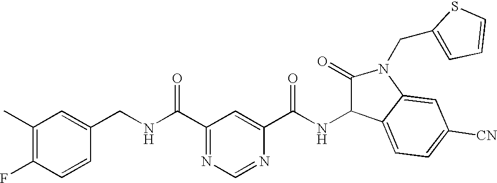 | 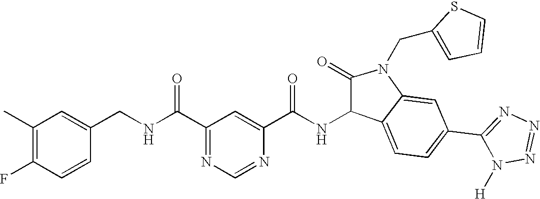 |
| 86 | 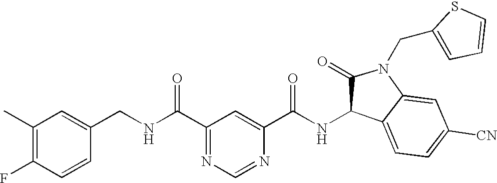 | 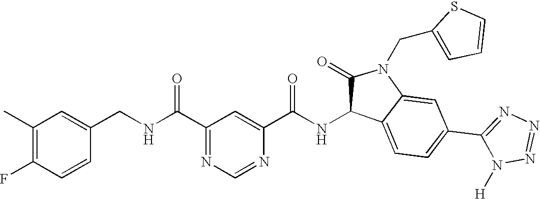 |
| 87 | 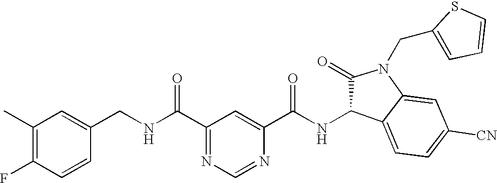 | 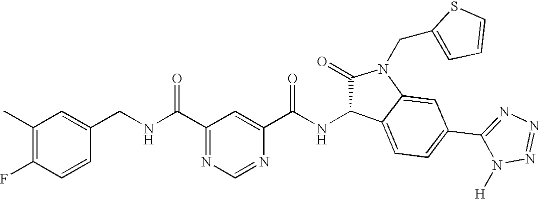 |
| 88 | 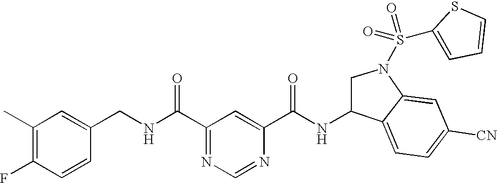 | 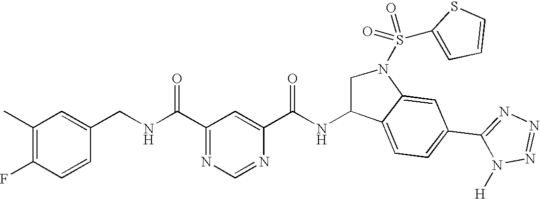 |
| 89 | 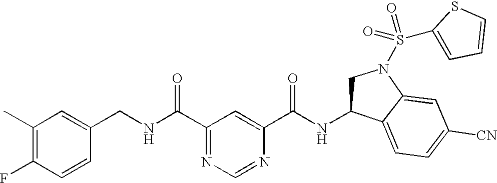 | 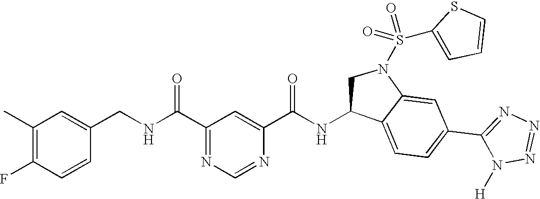 |
| 90 | 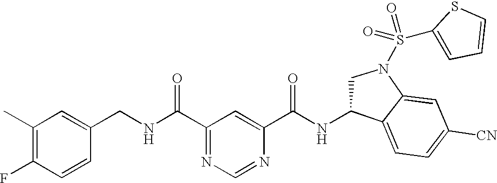 | 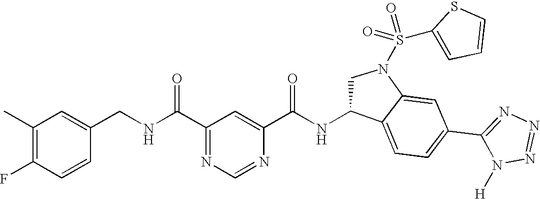 |
| 91 | 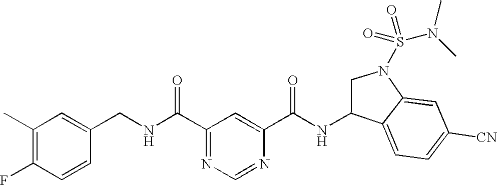 | 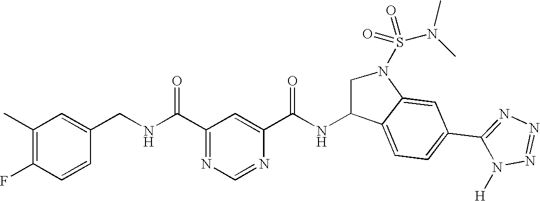 |
| 92 | 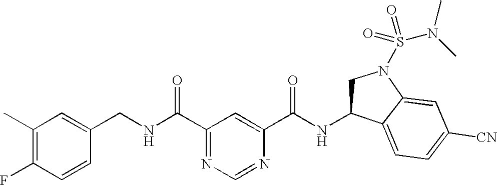 | 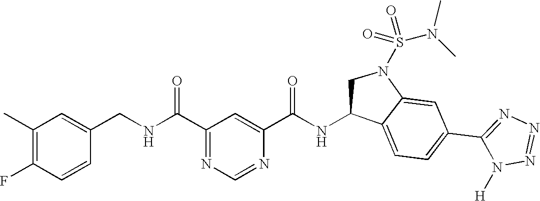 |
| 93 | 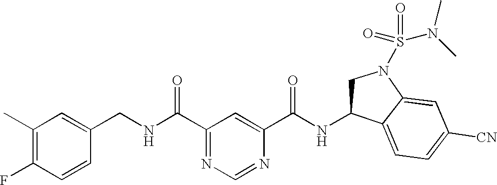 | 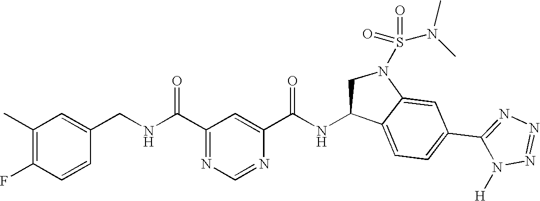 |
| 94 | 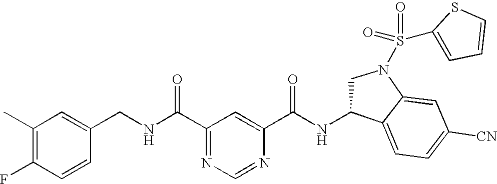 | 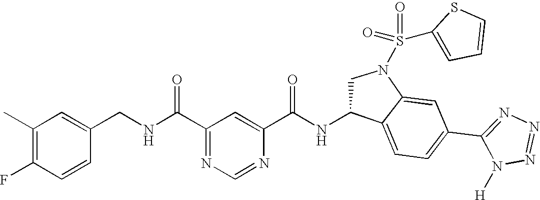 |
| 95 | 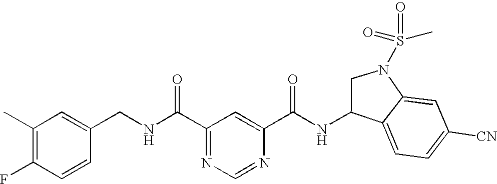 | 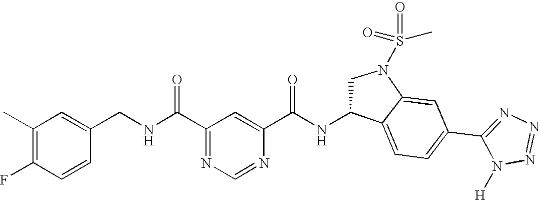 |
| 96 | 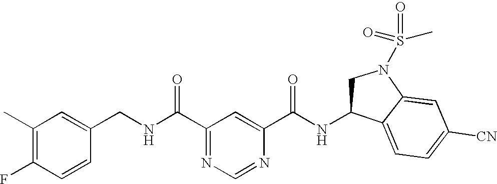 | 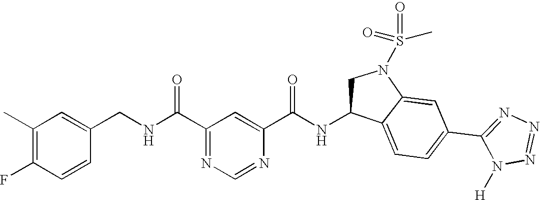 |
| 97 | 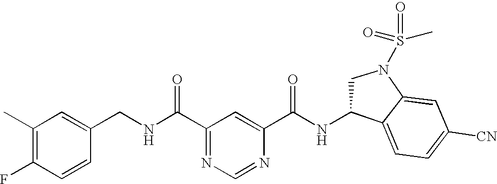 | 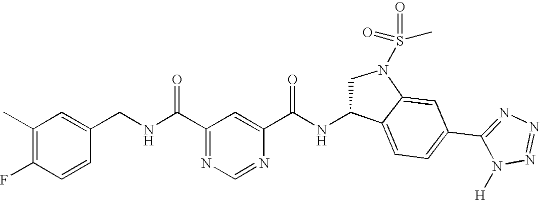 |
| 98 | 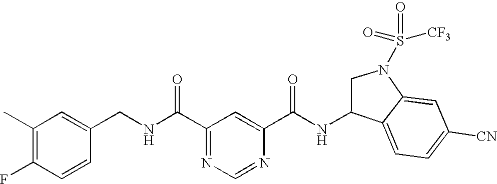 | 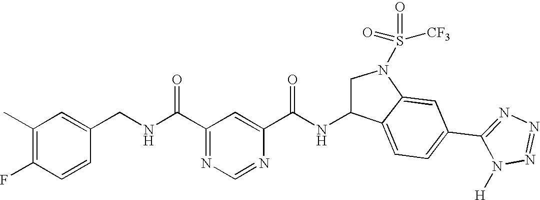 |
| 99 | 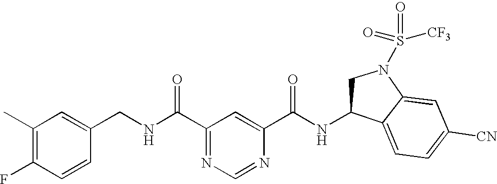 | 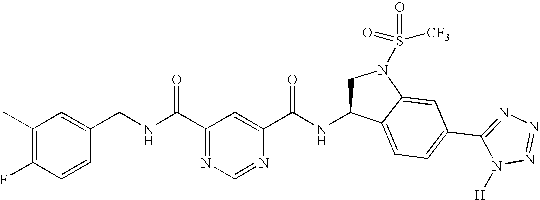 |
| 100 | 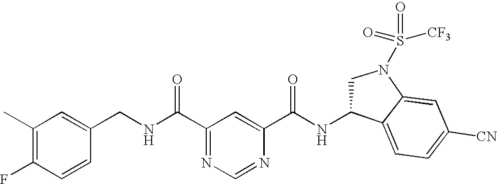 | 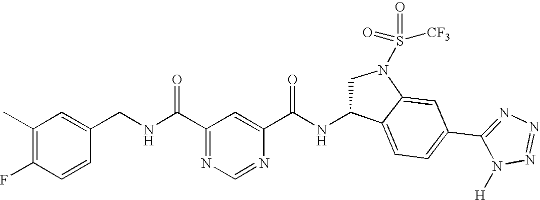 |
| 101 | 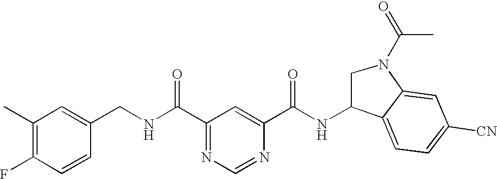 | 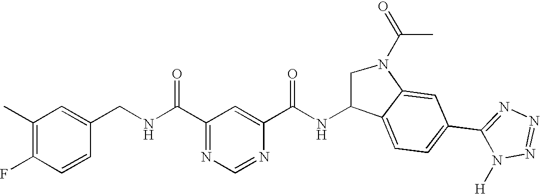 |
| 102 | 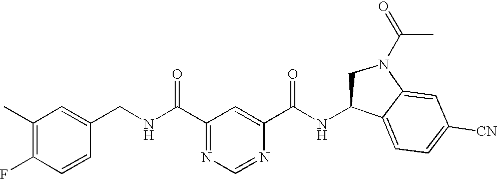 | 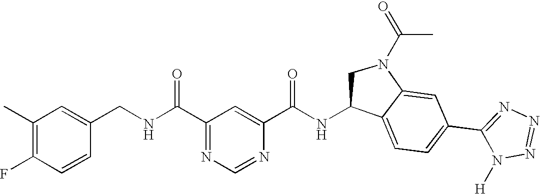 |
| 103 | 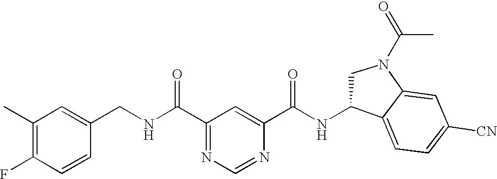 | 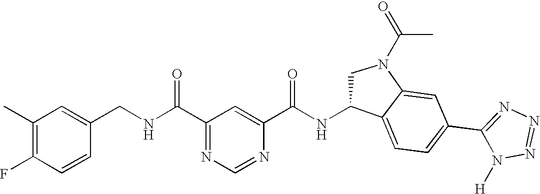 |
| 104 | 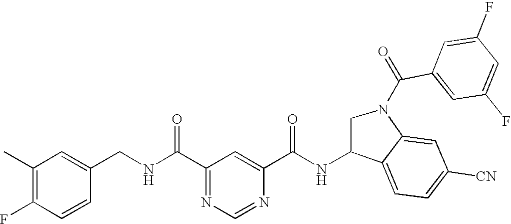 | 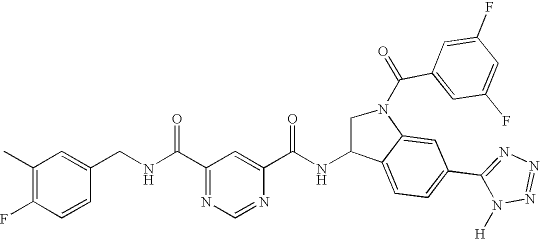 |
| 105 | 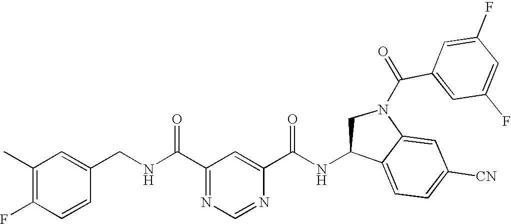 | 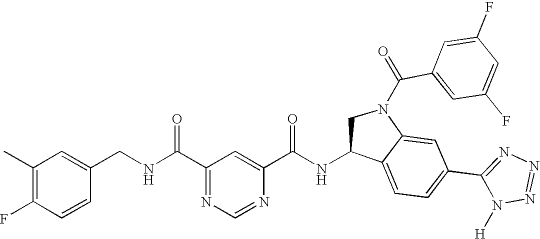 |
| 106 | 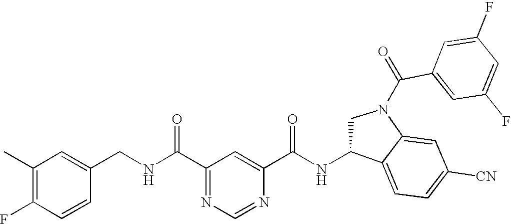 | 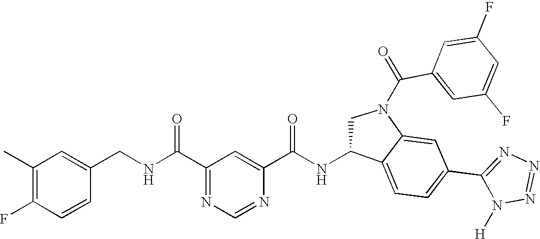 |
| 107 | 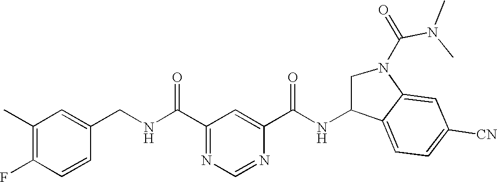 | 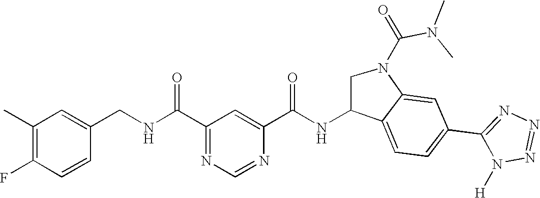 |
| 108 | 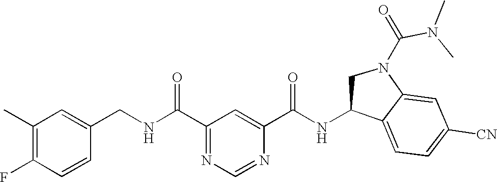 | 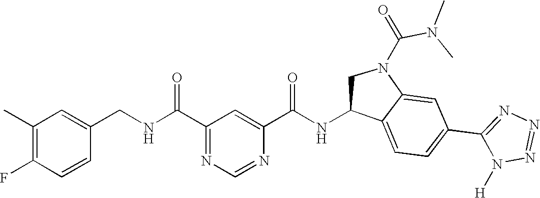 |
| 109 | 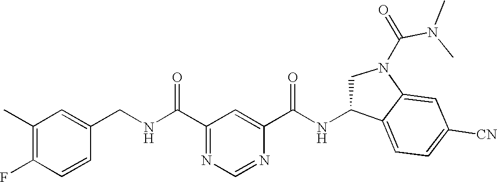 | 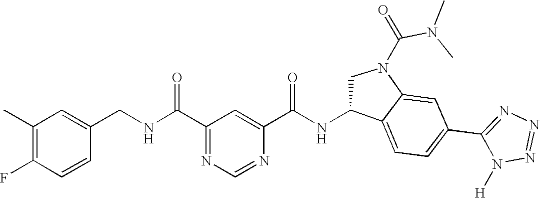 |
| 110 | 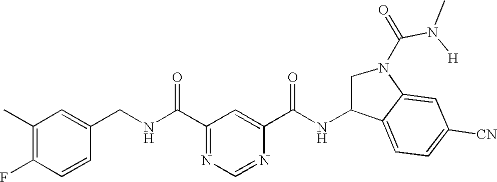 | 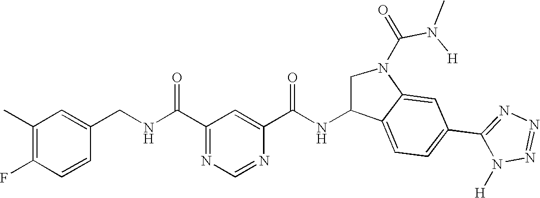 |
| 111 | 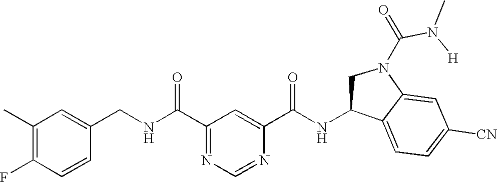 | 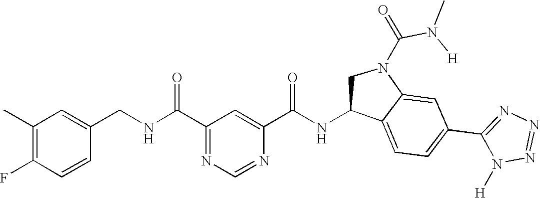 |
| 112 | 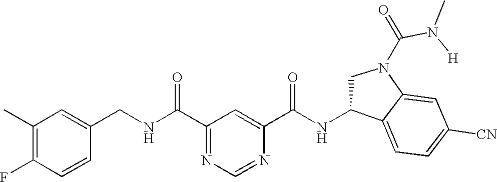 | 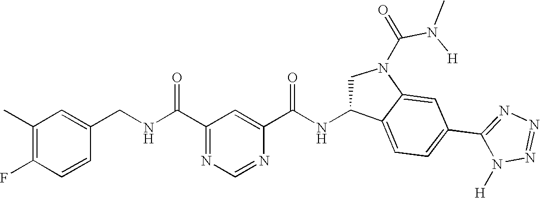 |
| 113 | 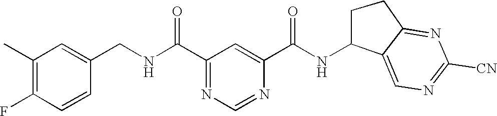 | 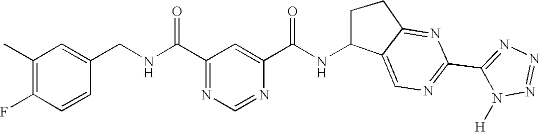 |
| 114 | 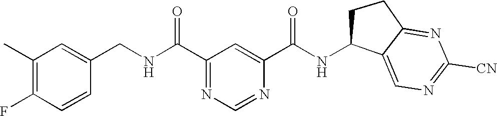 | 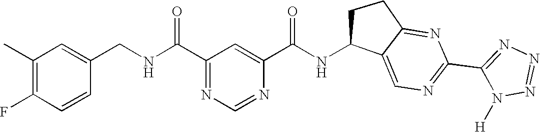 |
| 115 | 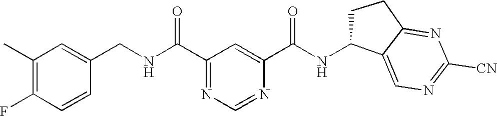 | 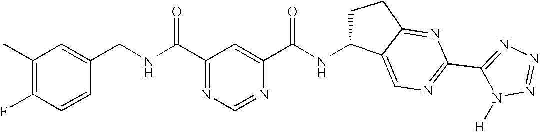 |
| 116 | 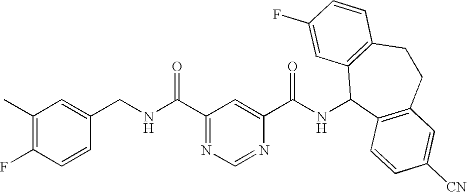 | 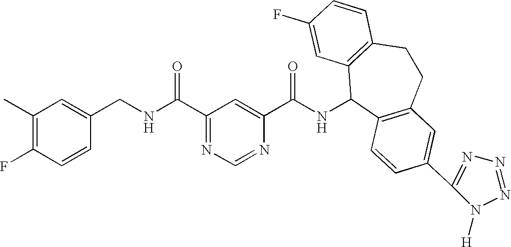 |
| 117 | 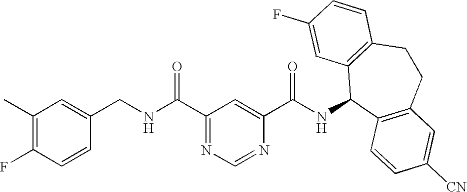 | 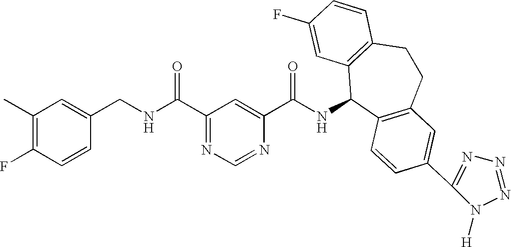 |
| 118 | 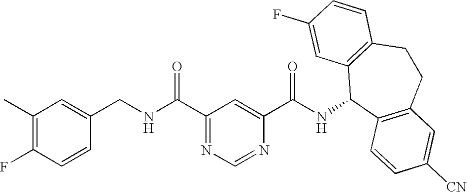 | 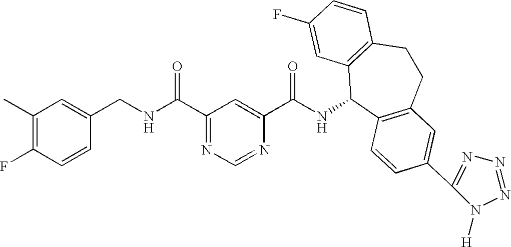 |
| 119 | 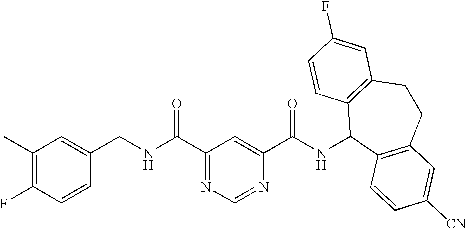 | 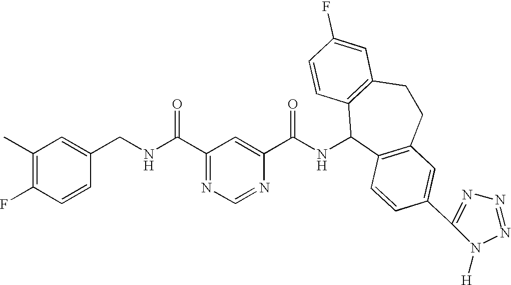 |
| 120 | 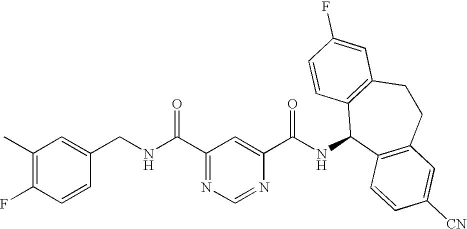 | 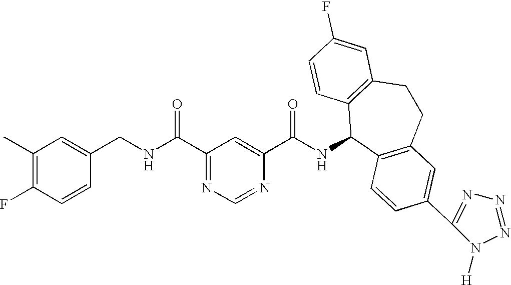 |
| 121 | 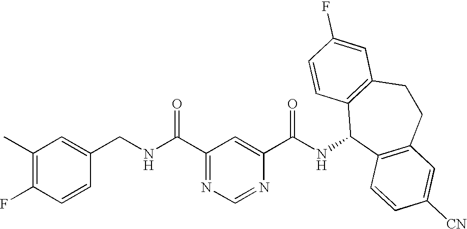 | 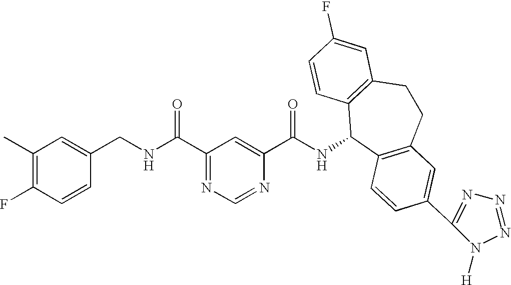 |
| 122 | 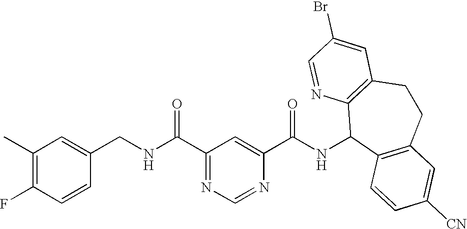 | 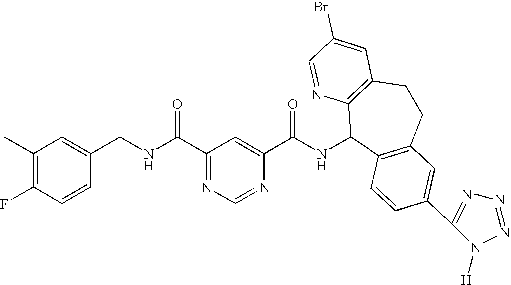 |
| 123 | 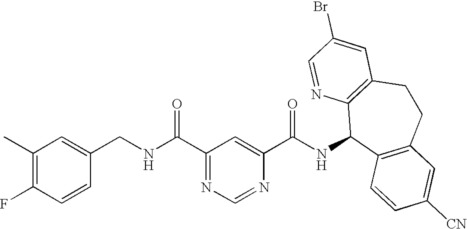 | 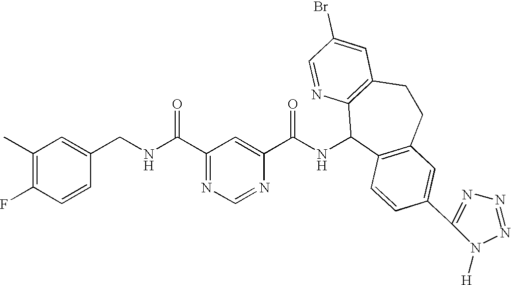 |
| 124 | 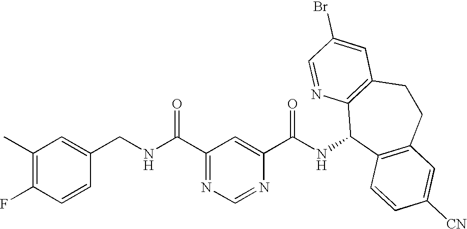 | 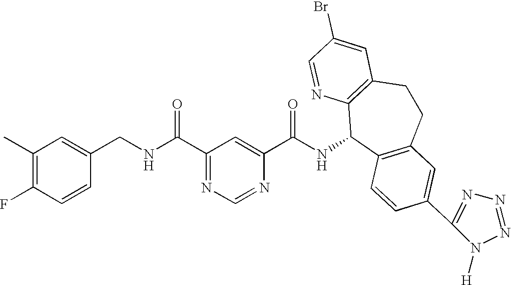 |
| 125 | 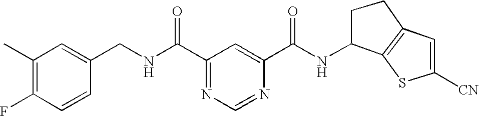 | 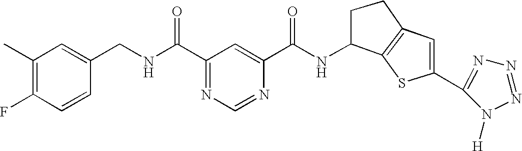 |
| 126 | 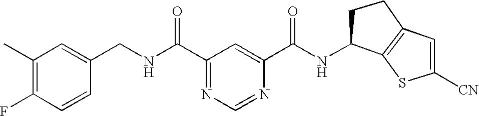 | 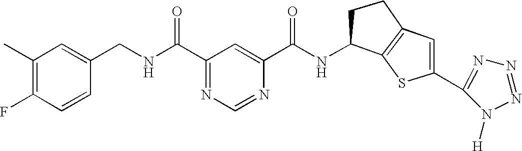 |
| 127 | 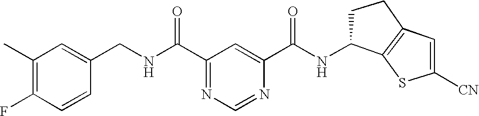 | 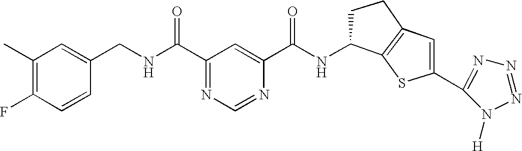 |
| 129 | 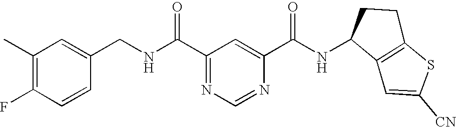 | 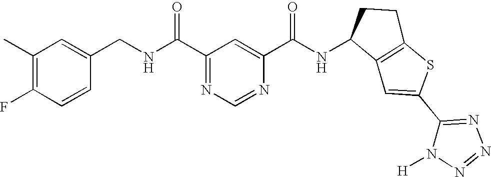 |
| 130 | 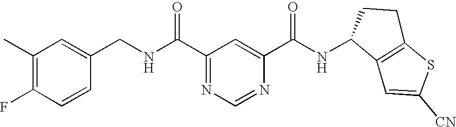 | 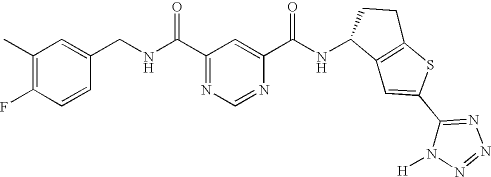 |
| 132 | 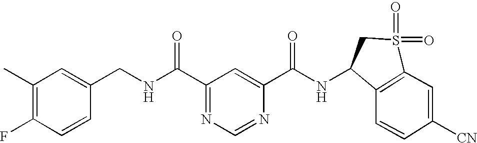 | 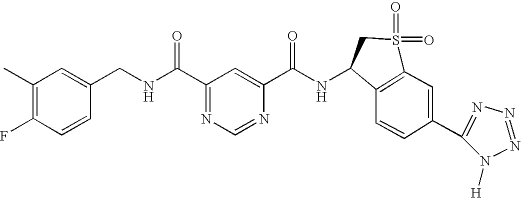 |
| 133 | 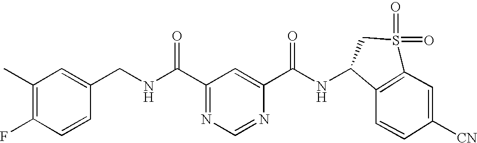 | 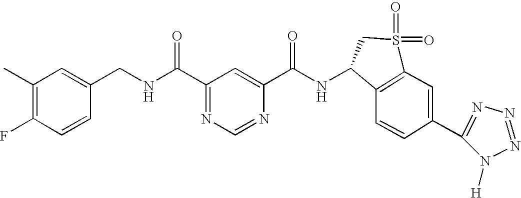 |
| 135 | 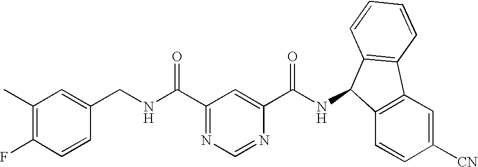 | 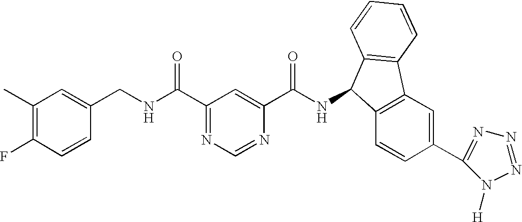 |
| 136 | 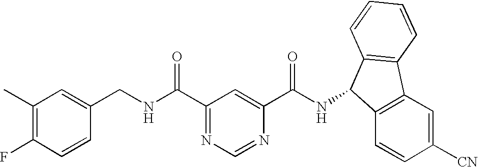 | 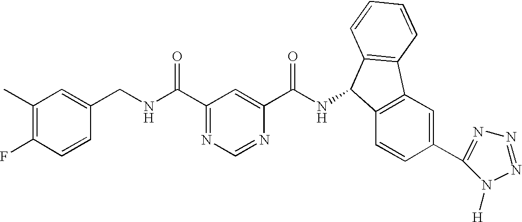 |
| 137 | 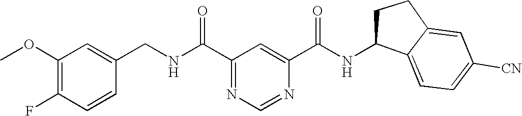 | 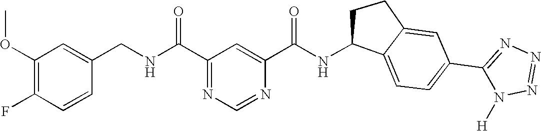 |
| 138 | 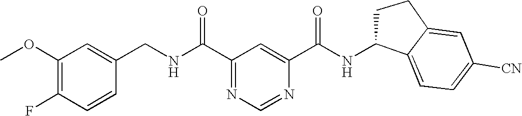 | 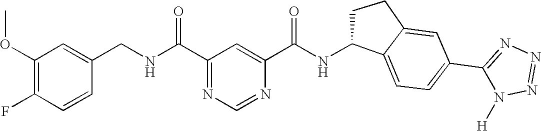 |
| 139 | 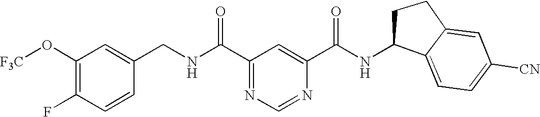 | 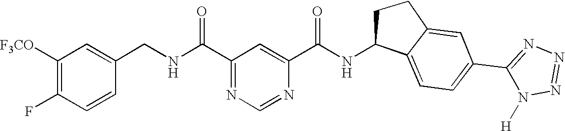 |
| 140 | 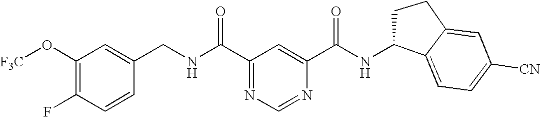 | 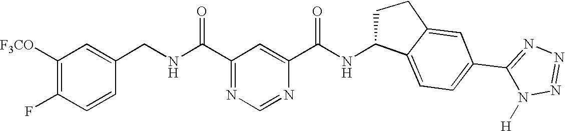 |
| 141 | 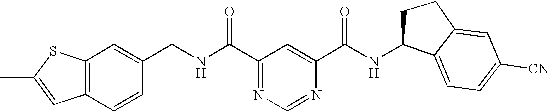 | 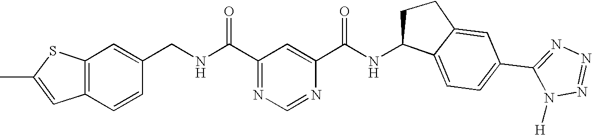 |
| 142 | 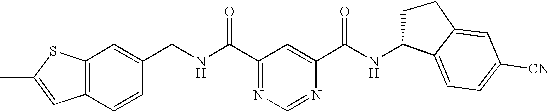 | 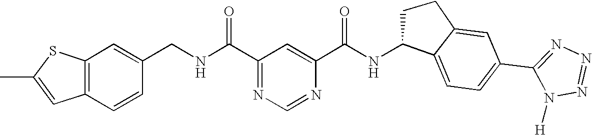 |
| 143 | 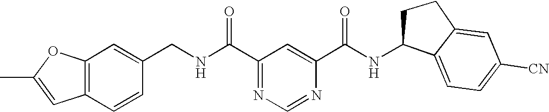 | 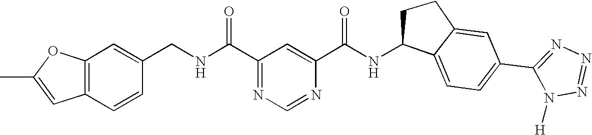 |
| 144 | 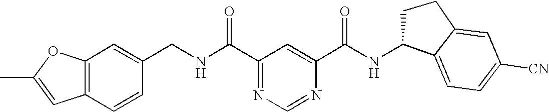 | 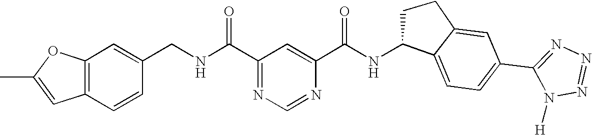 |
| 145 | 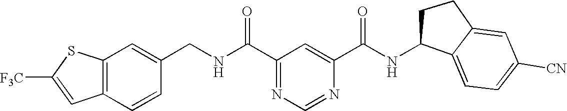 | 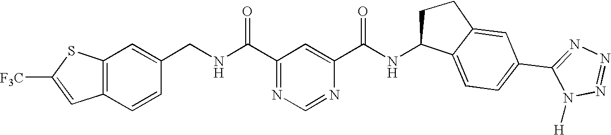 |
| 146 | 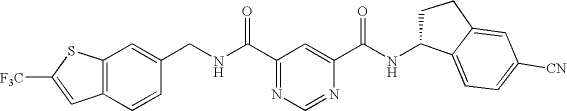 | 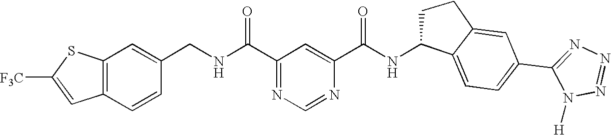 |
| 147 | 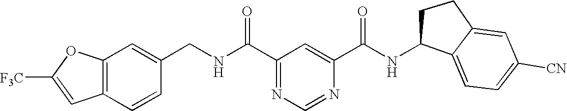 | 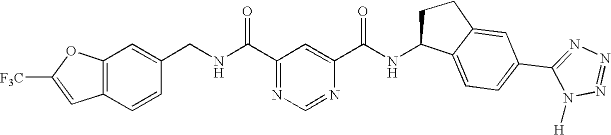 |
| 148 | 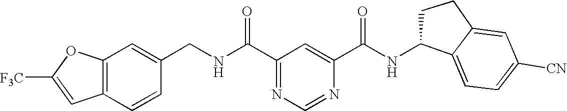 | 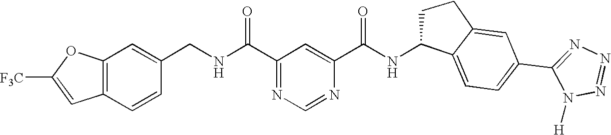 |
| 149 | 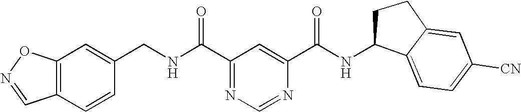 | 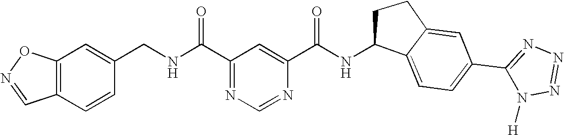 |
| 150 | 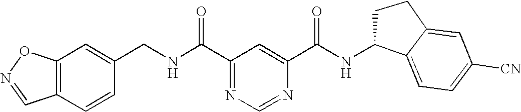 | 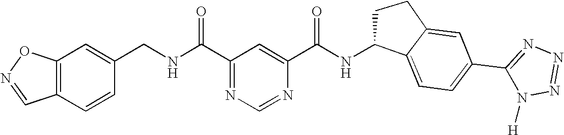 |
| 151 | 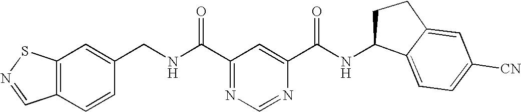 | 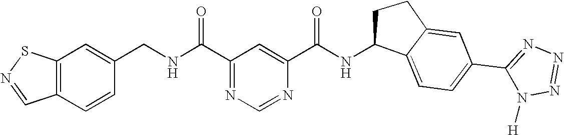 |
| 152 | 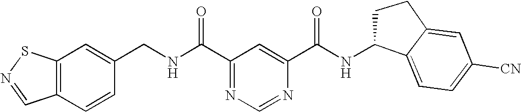 | 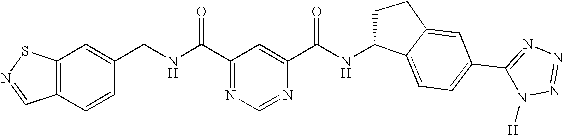 |

### Examples 201-230

If one were to treat the tetrazoles indicated in Table 5 below with a suitable base and methyliodide, one would obtain the methylated tetrazoles indicated.

TABLE 5

| Ex. # | Starting Material | Product |
|---|---|---|
| 201 | 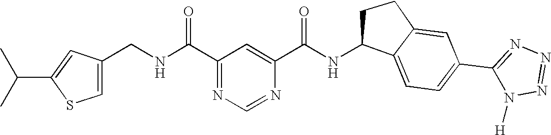 | 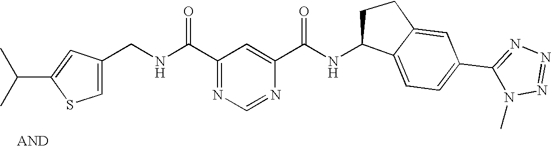 |
| | | 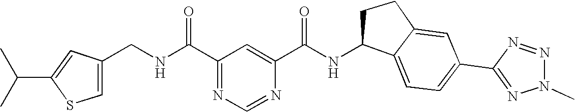 |
| 202 | 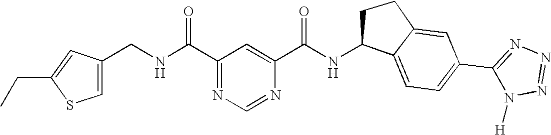 | 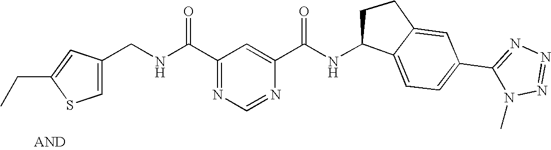 |
| | | 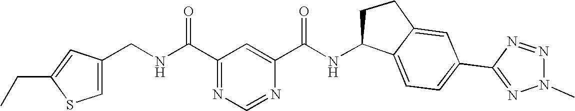 |
| 203 | 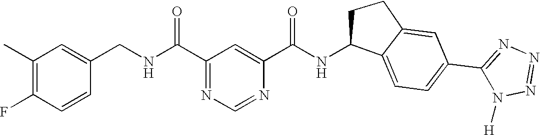 | 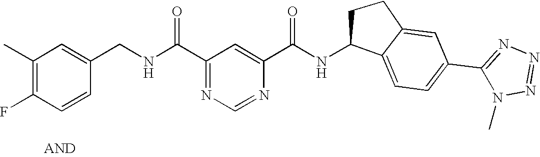 |
| | | 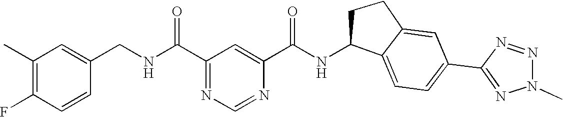 |
| 204 | 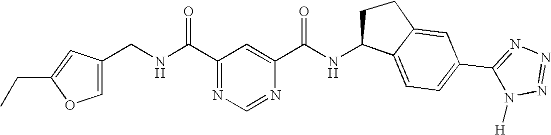 | 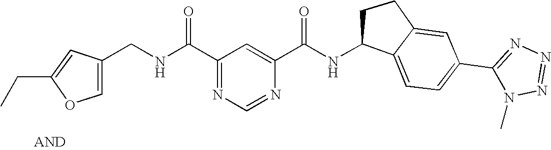 |
| | | 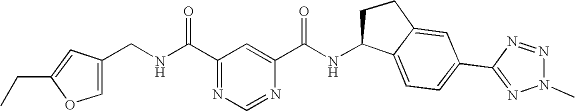 |
| 205 | 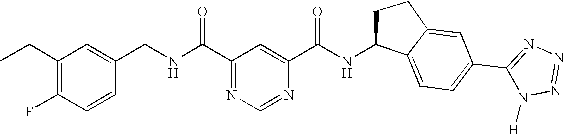 | 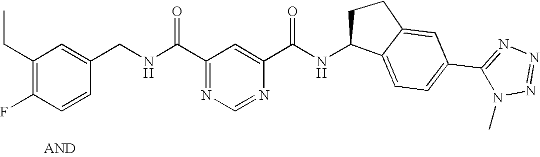 |
| | | 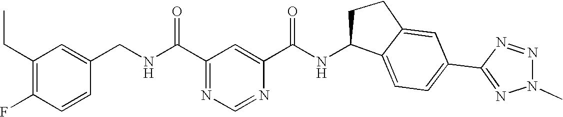 |
| 206 | 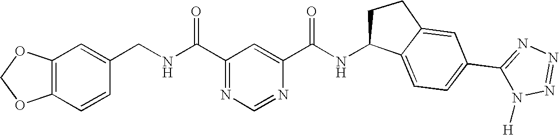 | 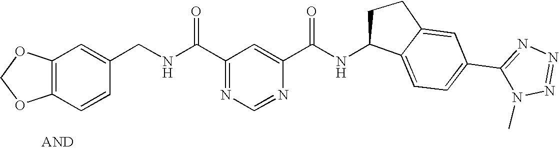 |
| | | 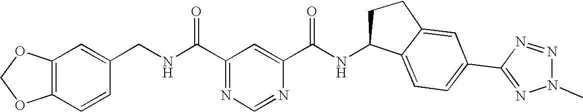 |
| 207 | 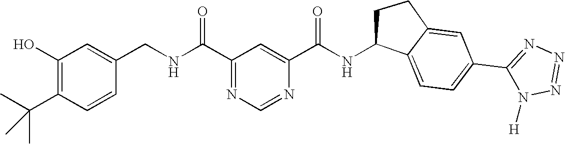 | 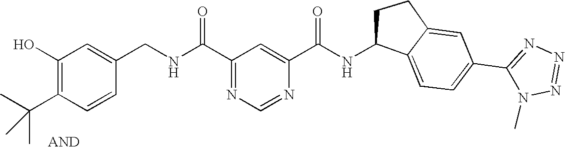 |
| | | 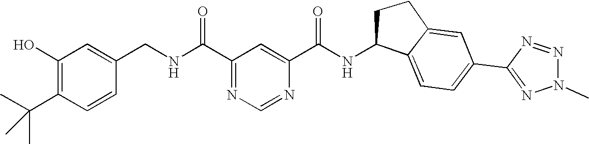 |
| 208 | 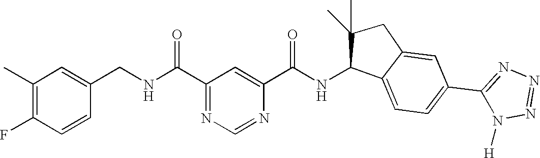 | 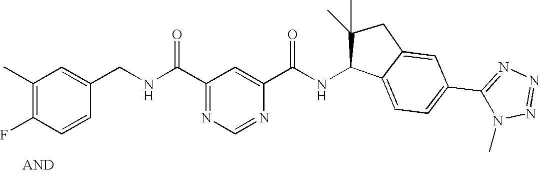 |
| | | 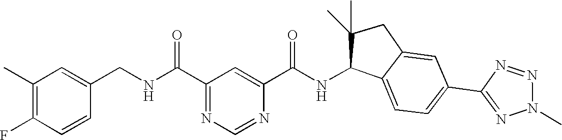 |
| 209 | 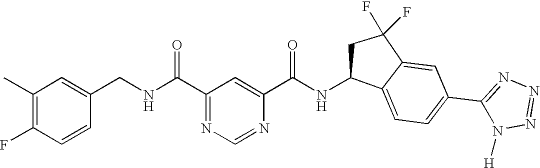 | 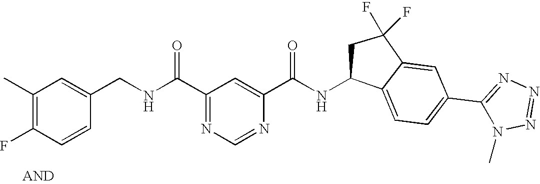 |
| | | 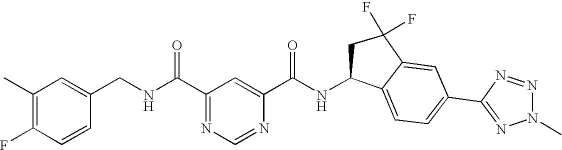 |
| 210 | 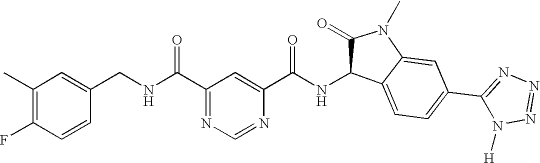 | 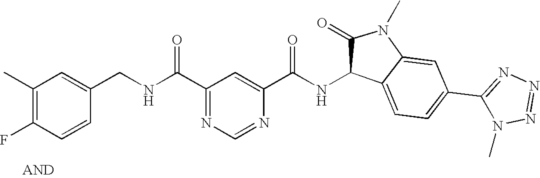 |
| | | 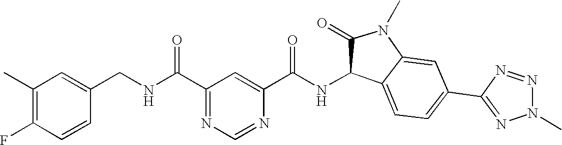 |
| 211 | 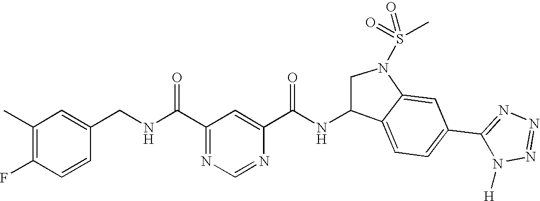 | 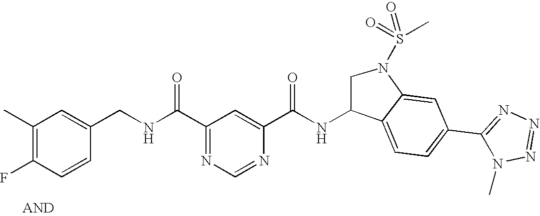 |
| | | 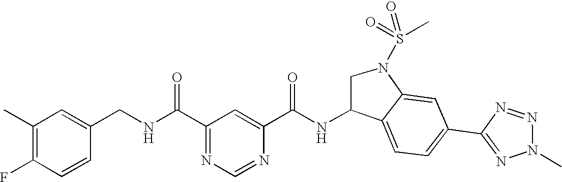 |
| 212 | 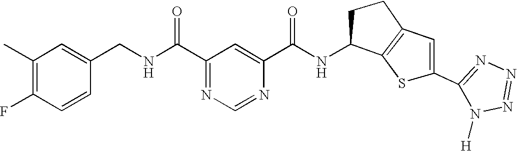 | 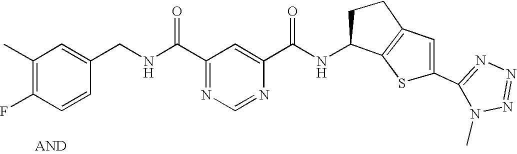 |
| | | 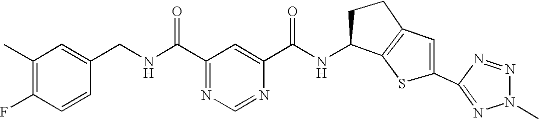 |
| 213 | 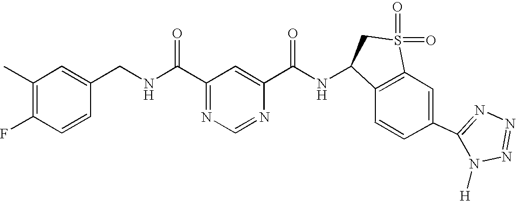 | 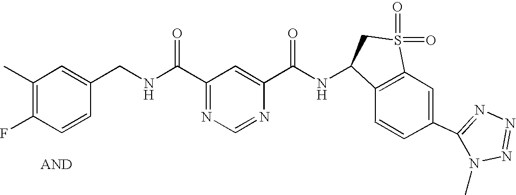 |
| | | 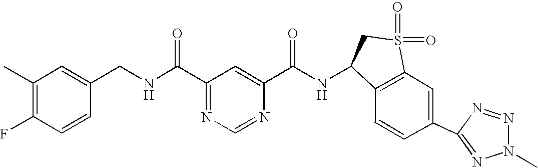 |
| 214 | 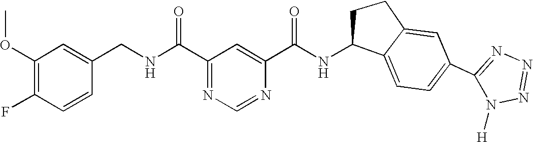 | 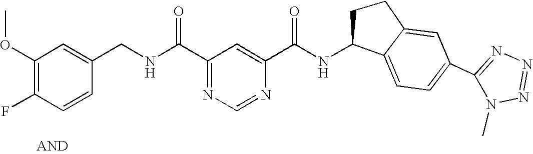 |
| | | 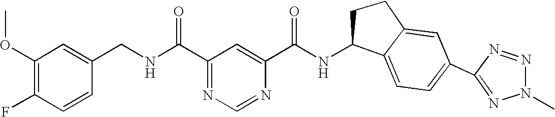 |
| 215 | 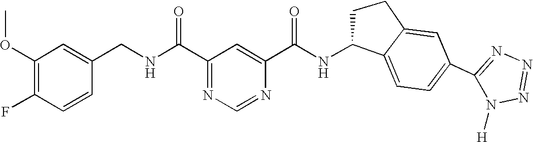 | 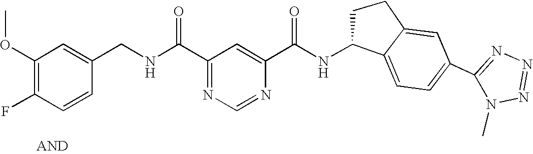 |
| | | 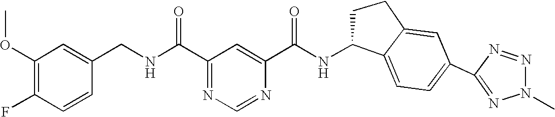 |
| 216 | 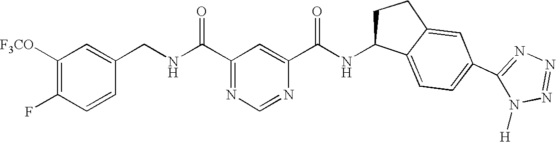 | 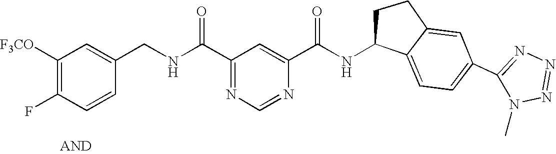 |
| | | 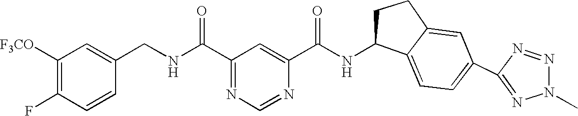 |
| 217 | 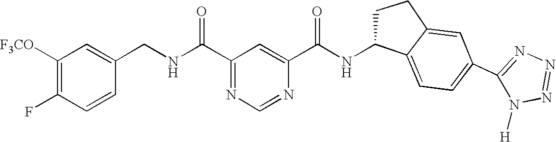 | 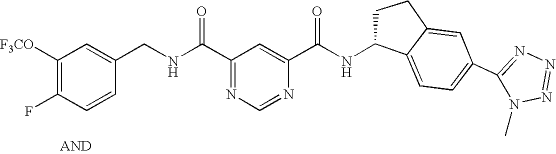 |
| | | 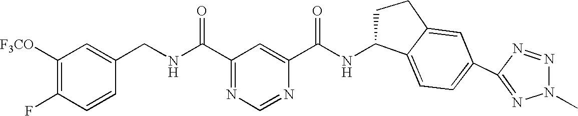 |
| 218 | 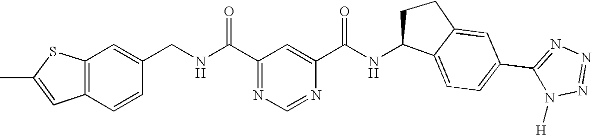 | 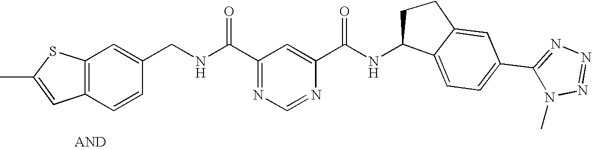 |
| | | 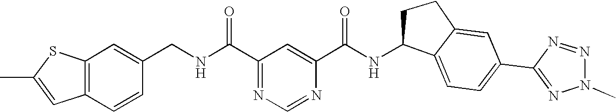 |
| 219 | 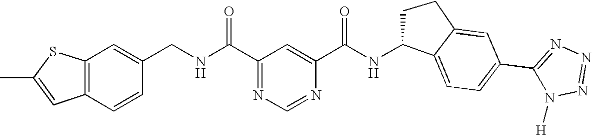 | 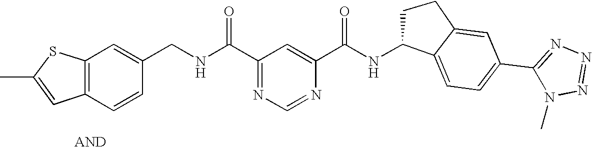 |
| | | 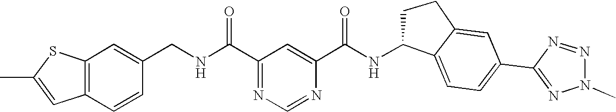 |
| 221 | 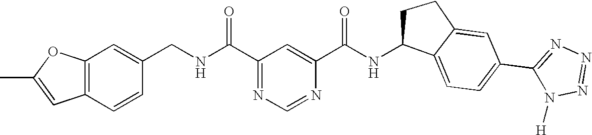 | 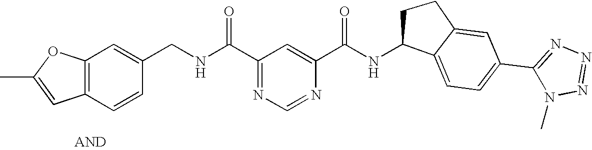 |
| | | 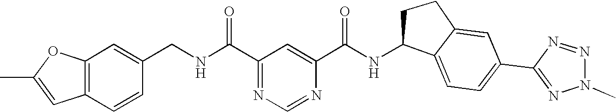 |
| 222 | 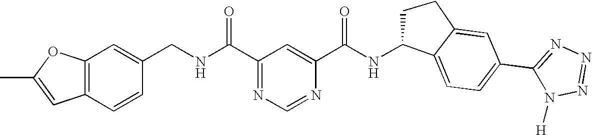 | 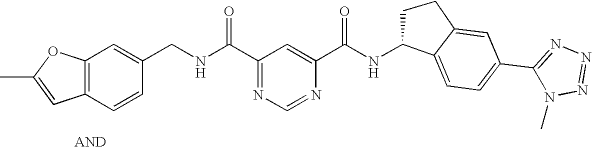 |
| | | 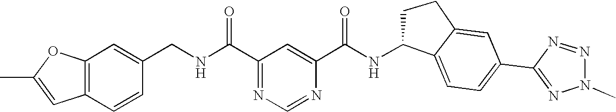 |
| 223 | 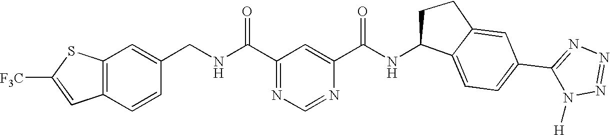 | 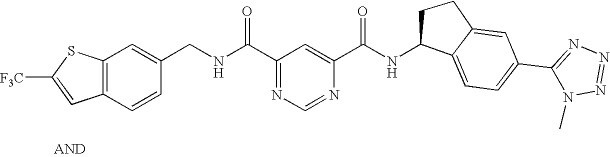 |
| | | 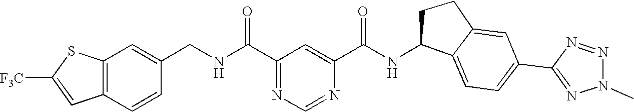 |
| 224 | 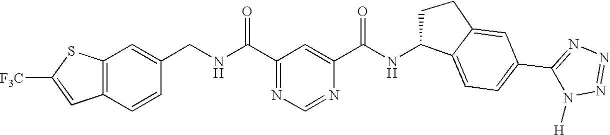 | 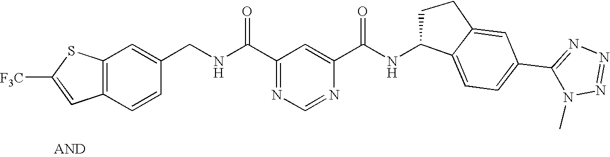 |
| | | 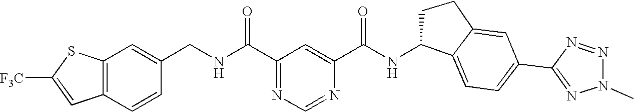 |
| 225 | 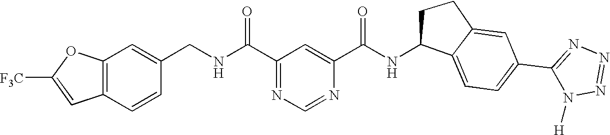 | 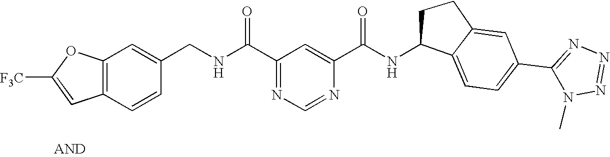 |
| | | 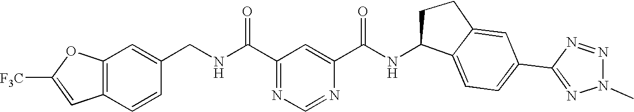 |
| 226 | 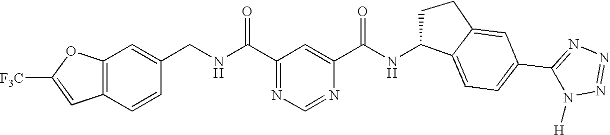 | 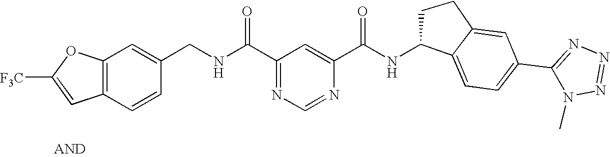 |
| | | 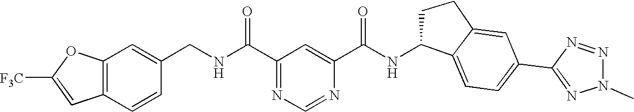 |
| 227 | 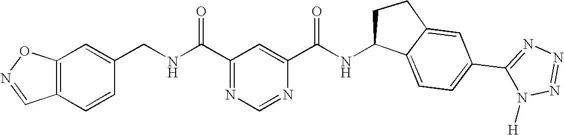 | 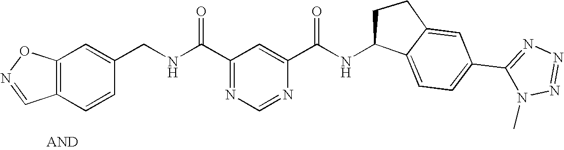 |
| | | 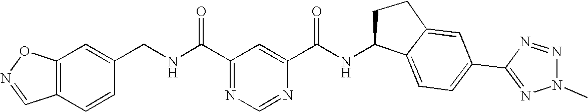 |
| 228 | 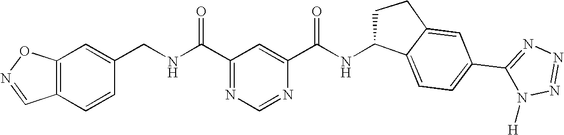 | 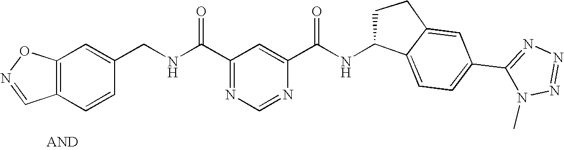 |
| | | 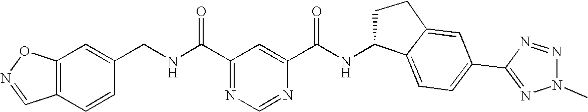 |
| 229 | 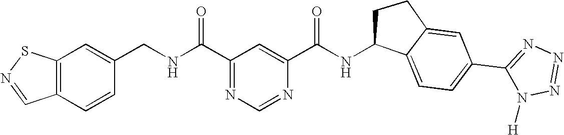 | 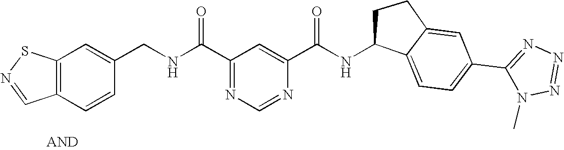 |
| | | 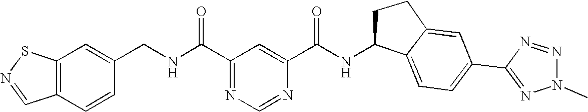 |
| 230 | 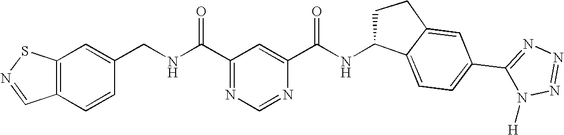 | 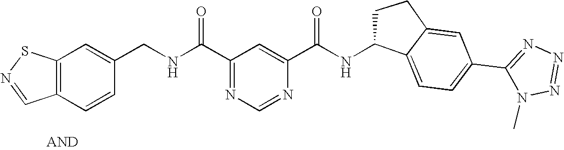 |
| | | 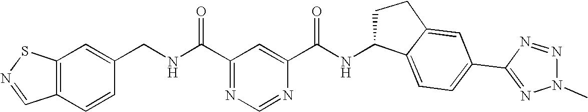 |

### Examples 301-330

If one were to follow similar procedures as described in Example 2500, Step A, with starting materials made according Preparative Example 2115 and Preparative Example 2121, one would obtain the desired compounds in Table 6 below.

TABLE 6

| Ex. # | Product |
|---|---|
| 301 | 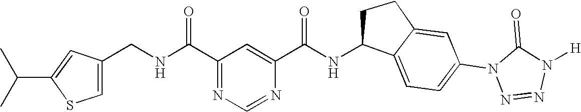 |
| 302 | 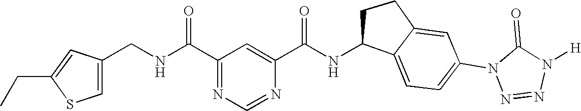 |
| 303 | 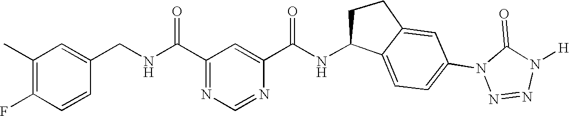 |
| 304 | 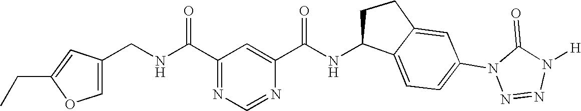 |
| 305 | 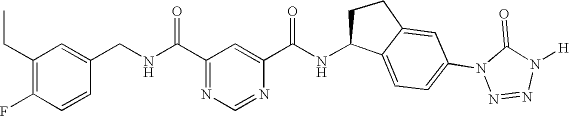 |
| 306 | 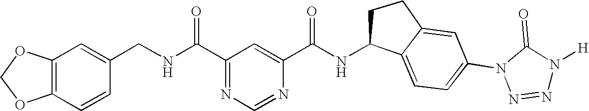 |
| 307 | 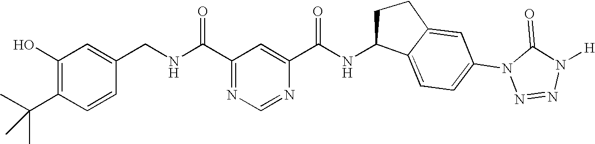 |
| 308 | 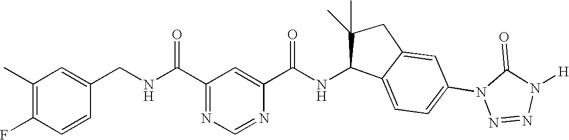 |
| 309 | 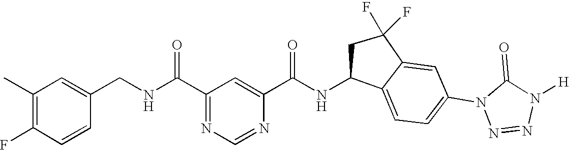 |
| 310 | 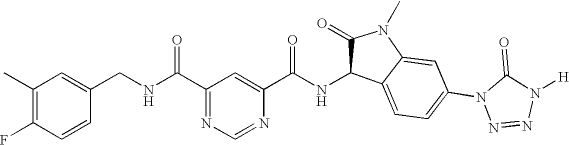 |
| 311 | 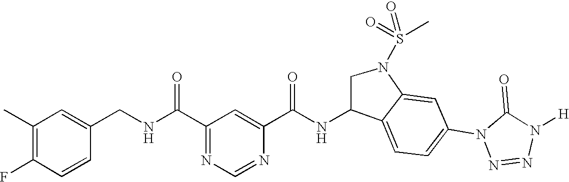 |
| 312 | 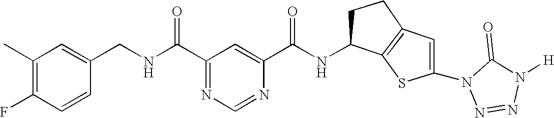 |
| 313 | 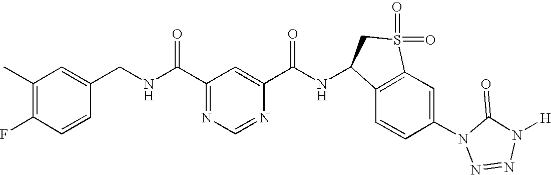 |
| 314 | 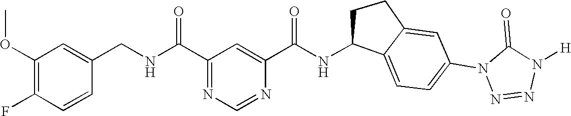 |
| 315 | 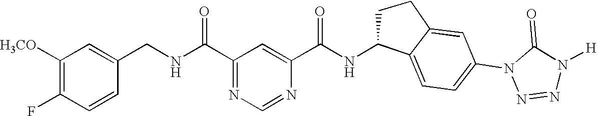 |
| 316 | 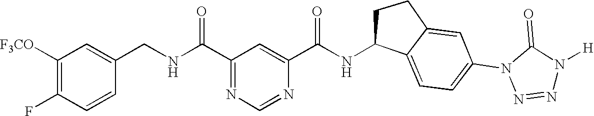 |
| 317 | 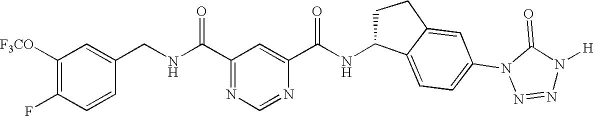 |
| 318 | 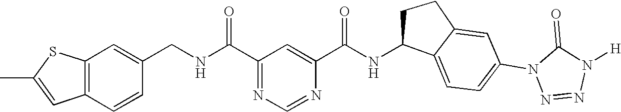 |
| 319 | 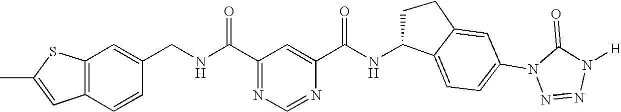 |
| 321 | 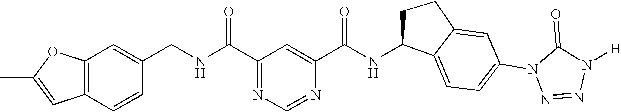 |
| 322 | 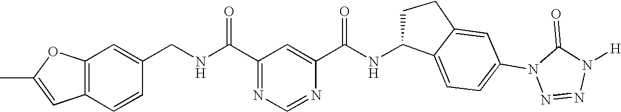 |
| 323 | 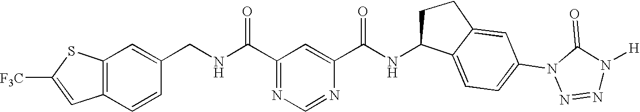 |
| 324 | 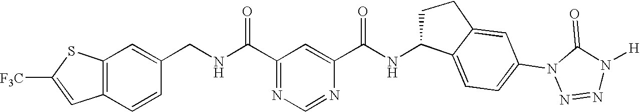 |
| 325 | 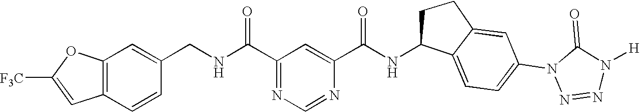 |
| 326 | 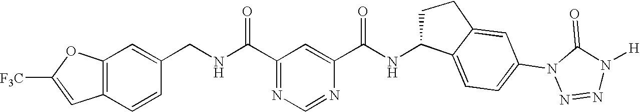 |
| 327 | 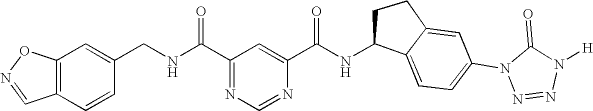 |
| 328 | 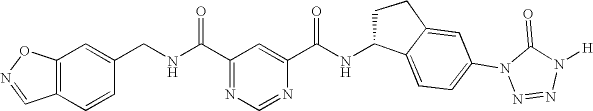 |
| 329 | 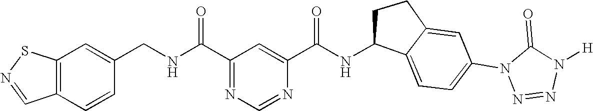 |
| 330 | 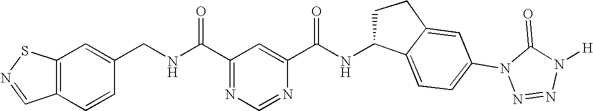 |

### Examples 401-430

If one were to heat the indicated hydroxytetrazoles in THF with a base (i.e. NaOH aq) and methliodide as described in the Preparative Example 2500, Step B, one would obtain the desired methylated tetrazole compounds in Table 7 below.

TABLE 7

| Ex. # | Starting Material | Product |
|---|---|---|
| 401 | 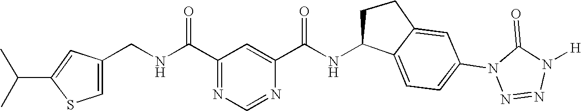 | 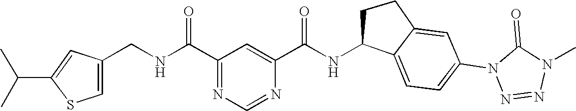 |
| 402 | 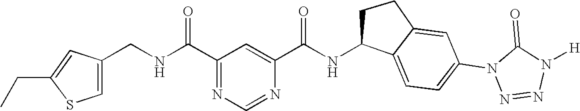 | 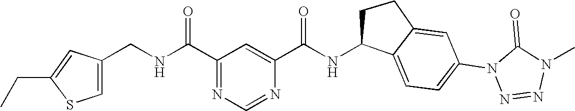 |
| 404 | 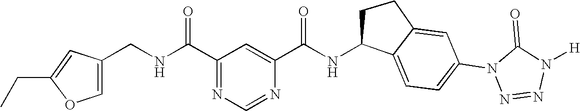 | 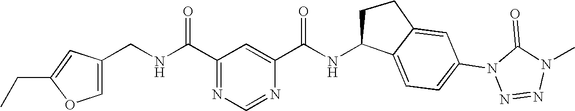 |
| 405 | 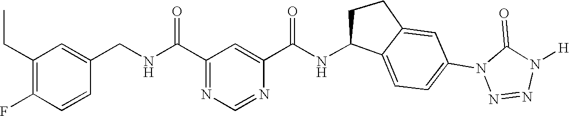 | 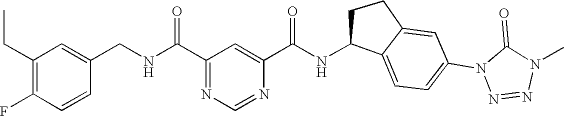 |
| 406 | 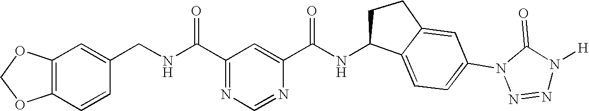 | 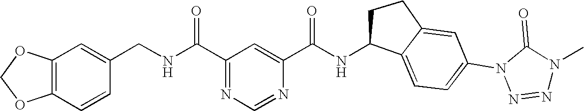 |
| 407 | 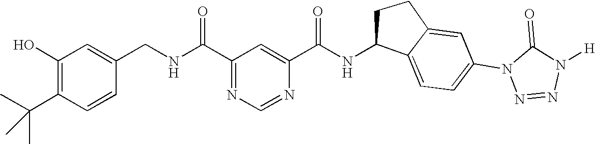 | 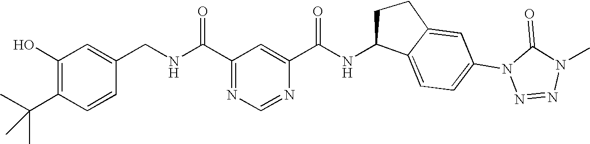 |
| 408 | 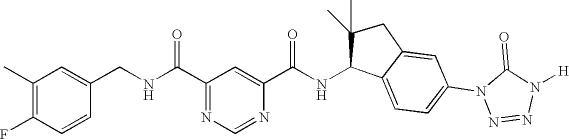 | 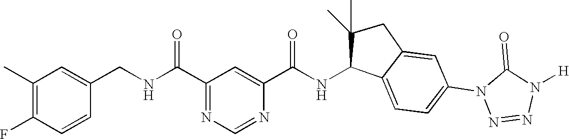 |
| 409 | 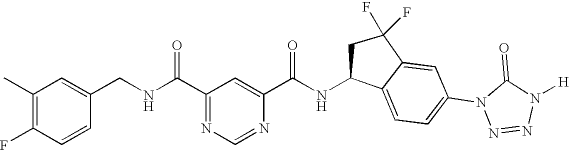 | 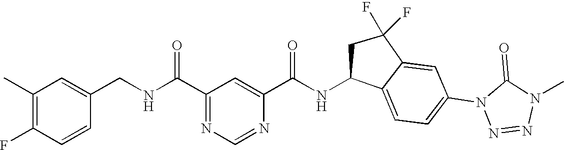 |
| 410 | 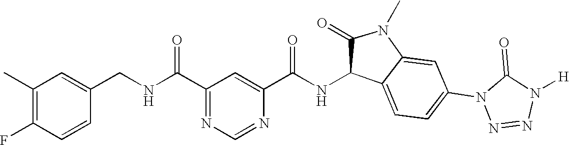 | 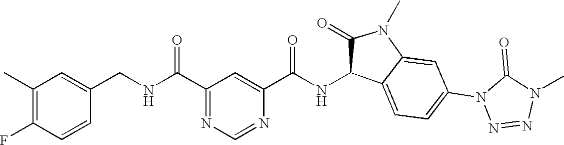 |
| 411 | 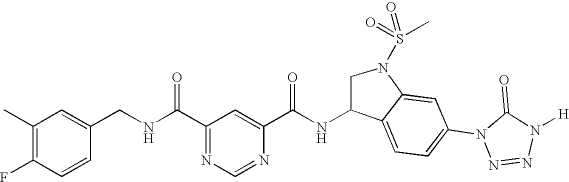 | 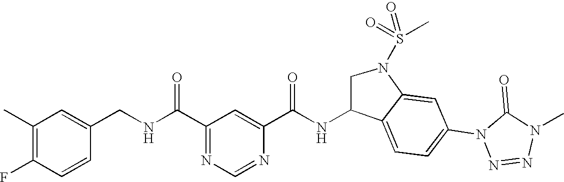 |
| 412 | 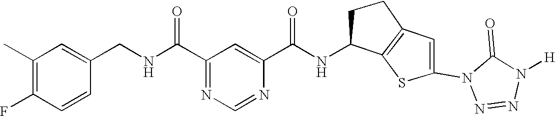 | 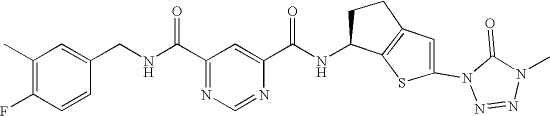 |
| 413 | 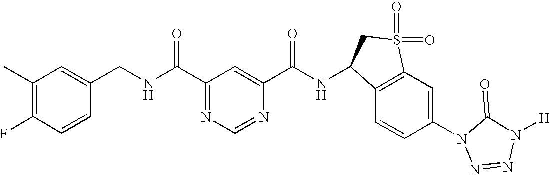 | 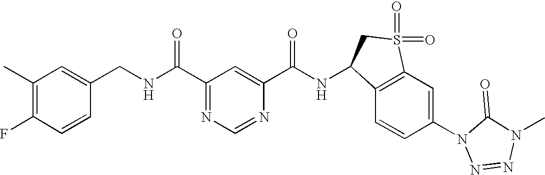 |
| 414 | 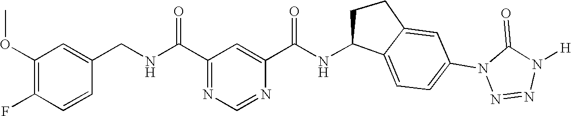 | 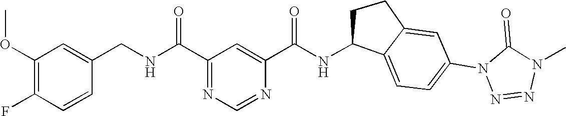 |
| 415 | 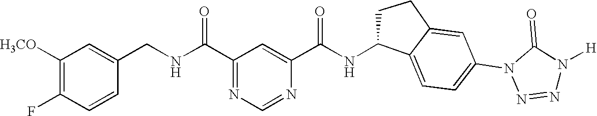 | 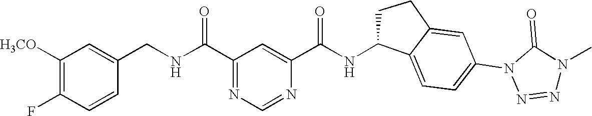 |
| 416 | 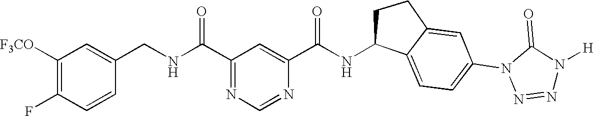 | 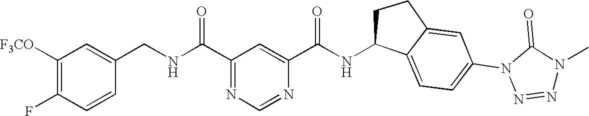 |
| 417 | 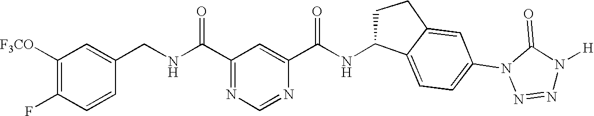 | 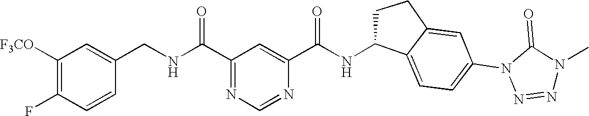 |
| 418 | 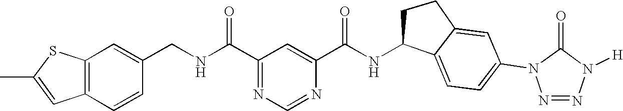 | 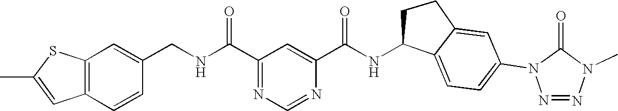 |
| 419 | 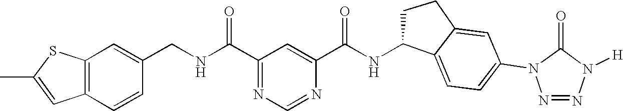 | 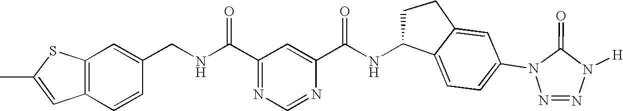 |
| 421 | 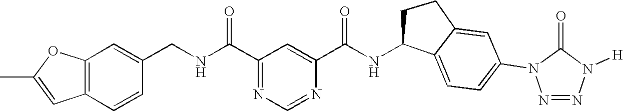 | 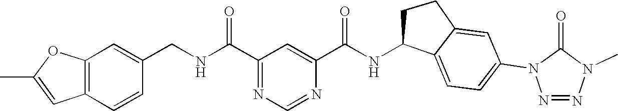 |
| 422 | 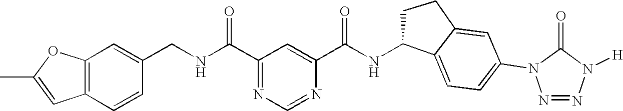 | 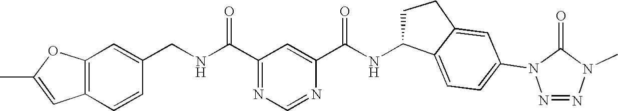 |
| 423 | 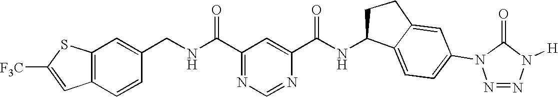 | 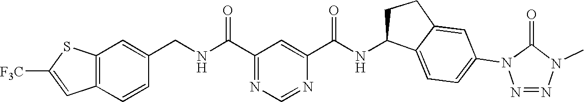 |
| 424 | 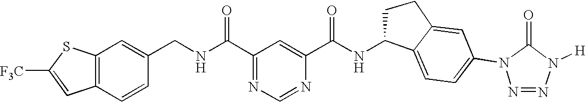 | 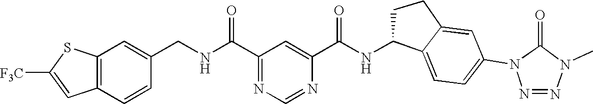 |
| 425 | 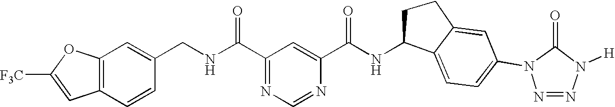 | 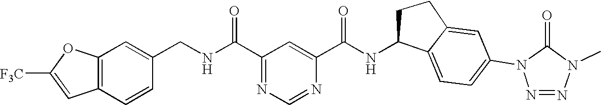 |
| 426 | 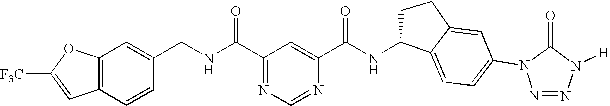 | 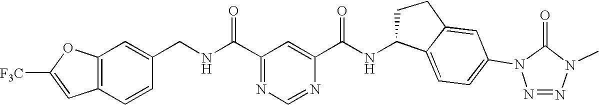 |
| 427 | 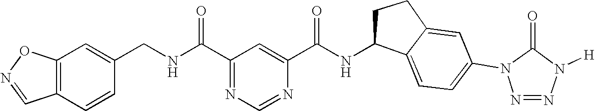 | 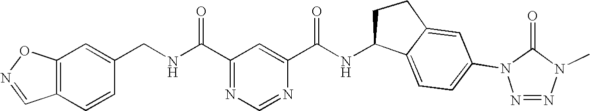 |
| 428 | 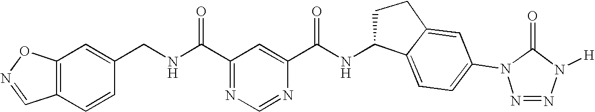 | 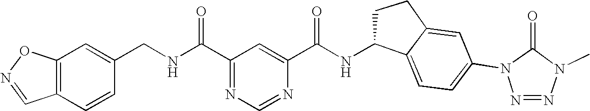 |
| 429 | 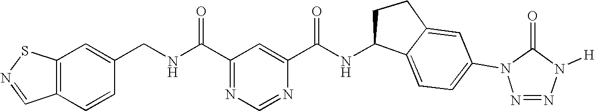 | 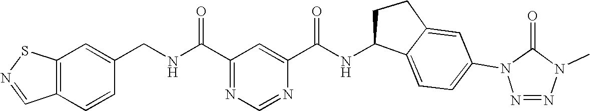 |
| 430 | 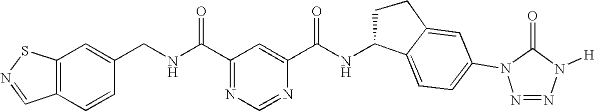 | 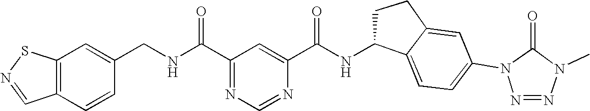 |

### Examples 501-530

If one were to heat the title compound from the list below according to similar procedures outlined in Preparative Example 9a and Example 9a, one would obtain the desired triazole, as shown in Table 8 below.

TABLE 8

| Ex. # | Starting Material | Product |
|---|---|---|
| 501 | 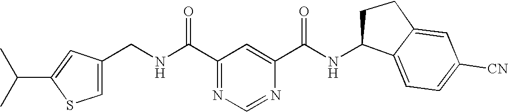 | 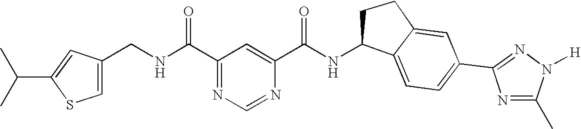 |
| 502 | 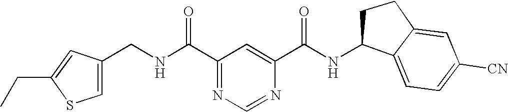 | 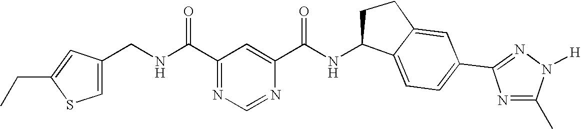 |
| 503 | 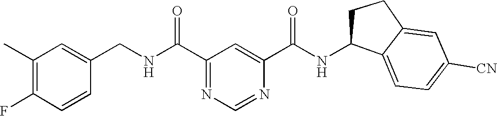 | 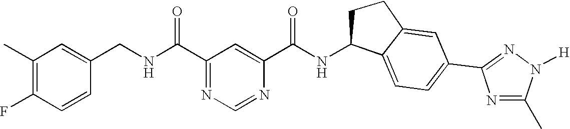 |
| 504 | 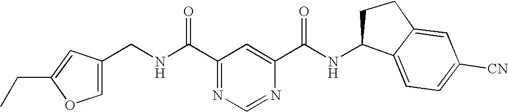 | 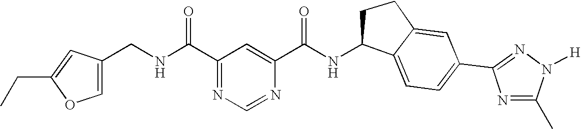 |
| 505 | 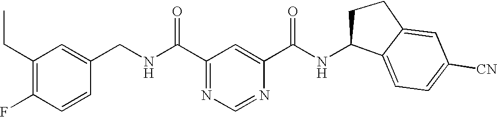 | 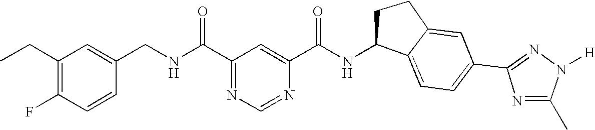 |
| 506 | 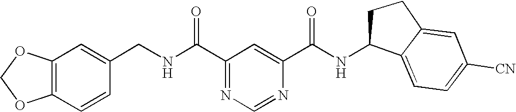 | 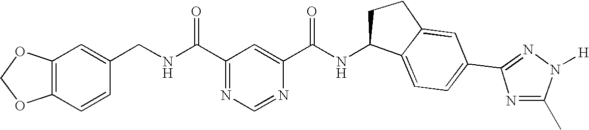 |
| 507 | 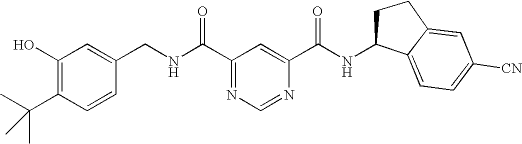 | 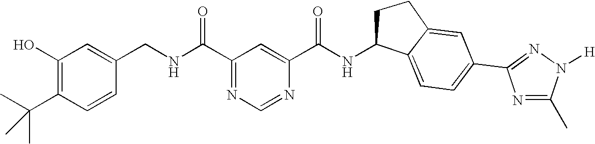 |
| 508 | 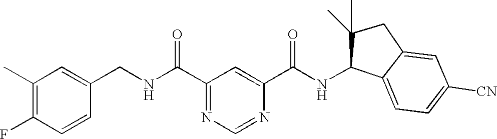 | 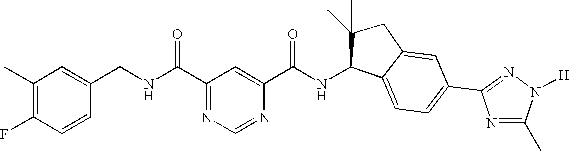 |
| 509 | 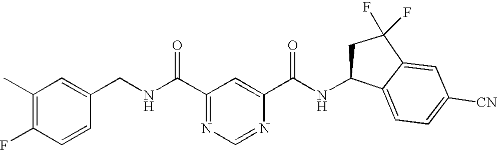 | 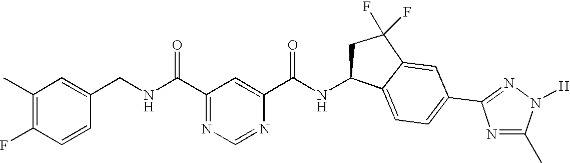 |
| 510 | 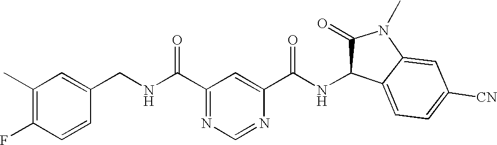 | 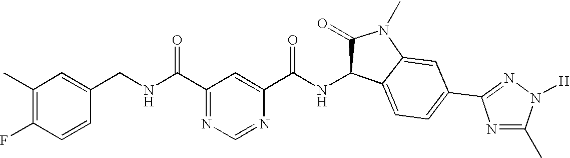 |
| 511 | 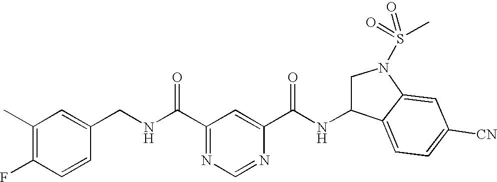 | 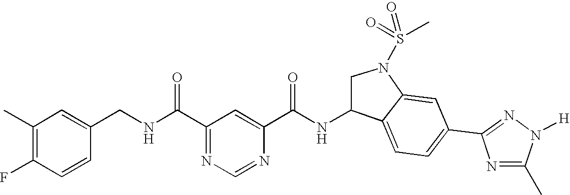 |
| 512 | 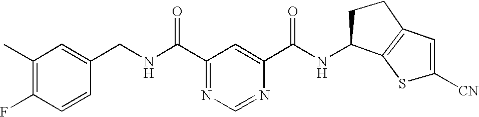 | 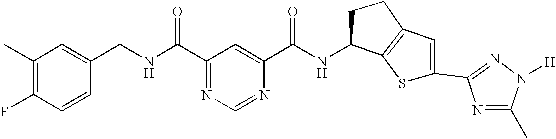 |
| 513 | 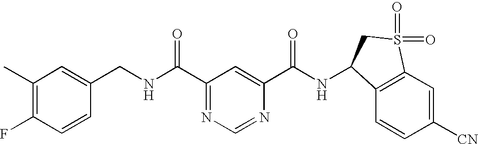 | 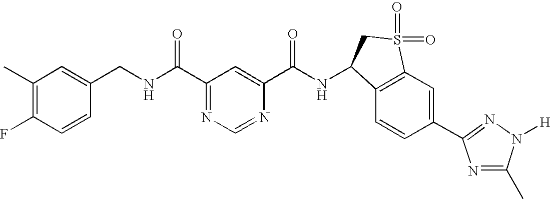 |
| 514 | 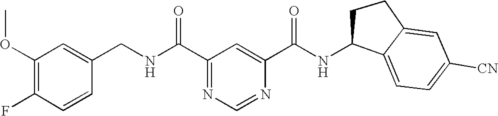 | 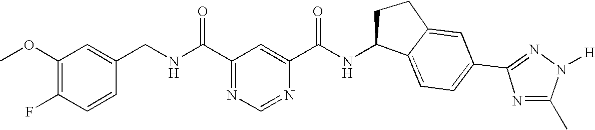 |
| 515 | 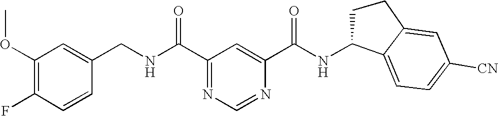 | 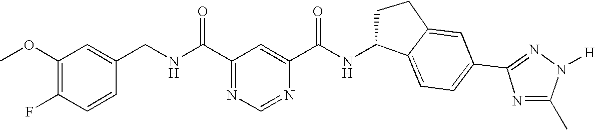 |
| 516 | 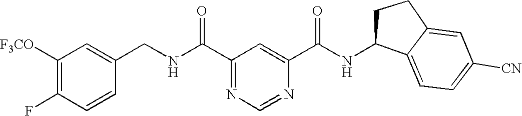 | 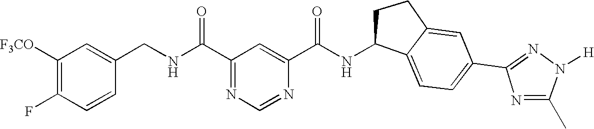 |
| 517 | 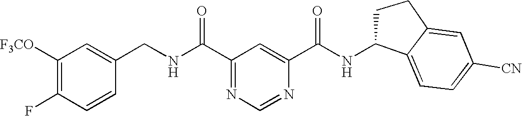 | 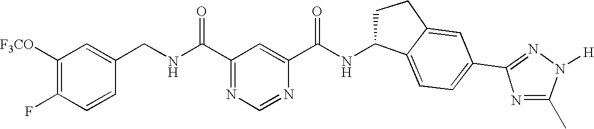 |
| 518 | 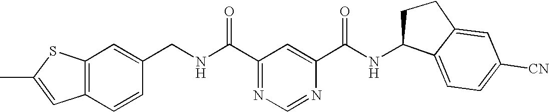 | 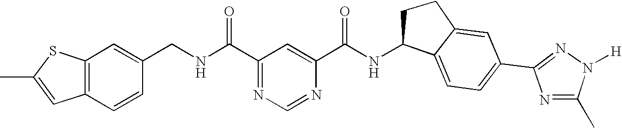 |
| 519 | 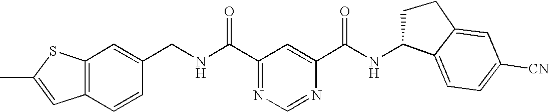 | 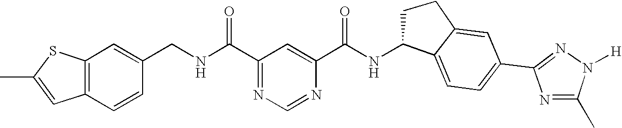 |
| 521 | 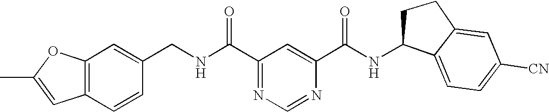 | 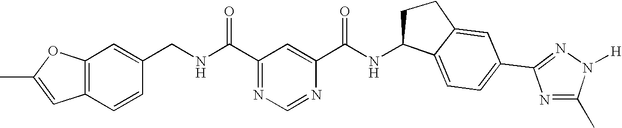 |
| 522 | 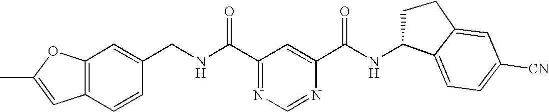 | 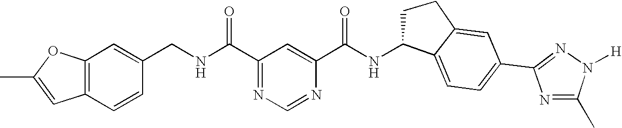 |
| 523 | 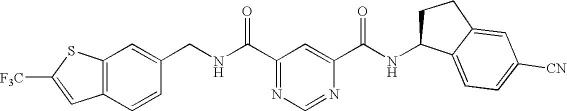 | 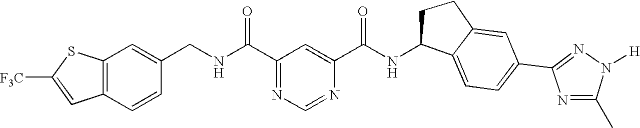 |
| 524 | 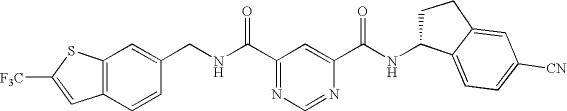 | 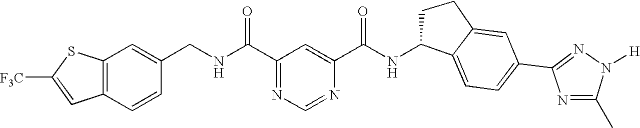 |
| 525 | 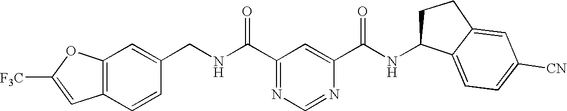 | 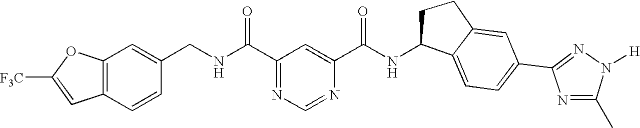 |
| 526 | 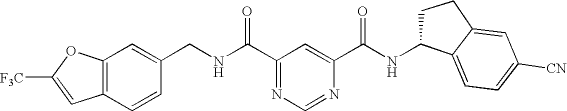 | 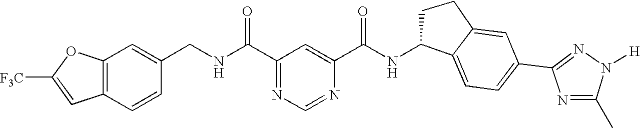 |
| 527 | 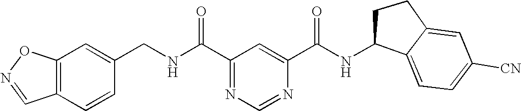 | 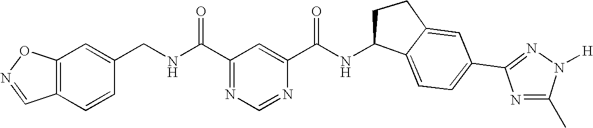 |
| 528 | 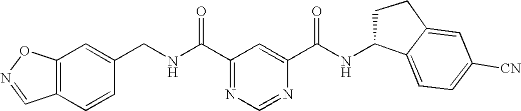 | 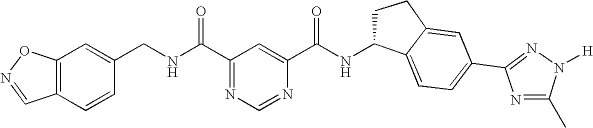 |
| 529 | 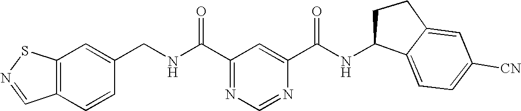 | 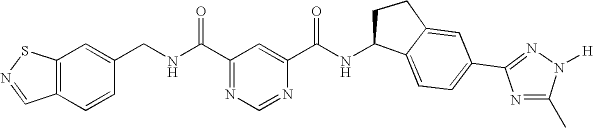 |
| 530 | 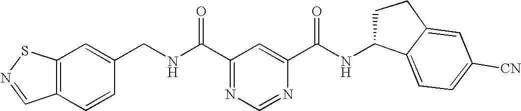 | 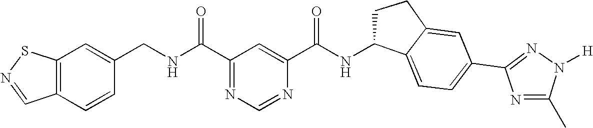 |

### Examples 601-630

If one were to heat the title compound from the list below according to similar procedures outlined in the Preparative Example 9a and Example 9b, one would obtain the desired triazole, as shown in Table 9 below.

TABLE 9

| Ex. # | Starting Material | Product |
|---|---|---|
| 601 | 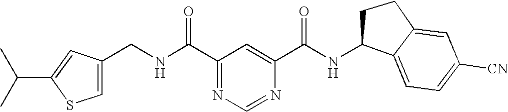 | 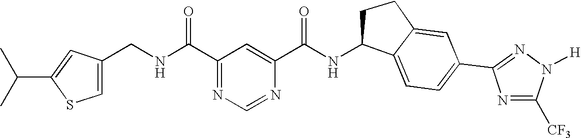 |
| 602 | 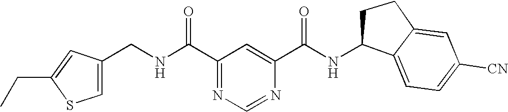 | 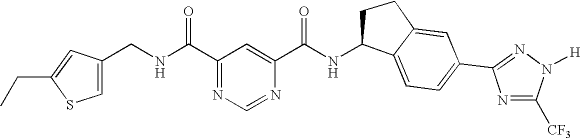 |
| 603 | 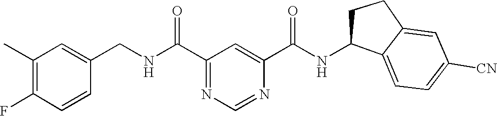 | 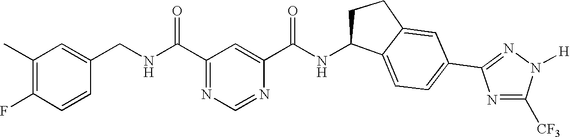 |
| 604 | 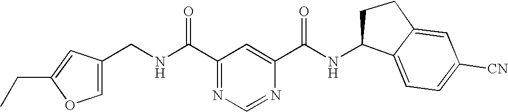 | 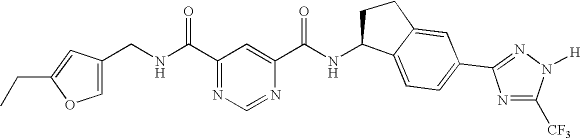 |
| 605 | 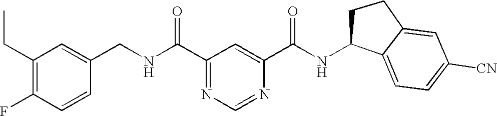 | 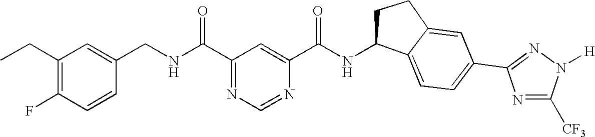 |
| 606 | 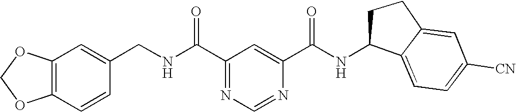 | 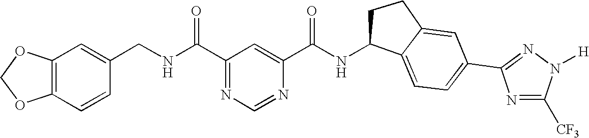 |
| 607 | 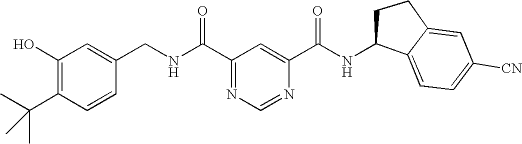 | 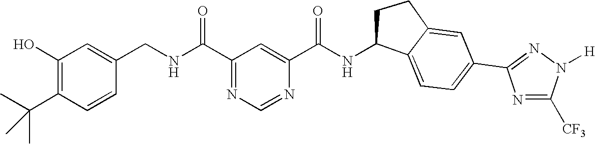 |
| 608 | 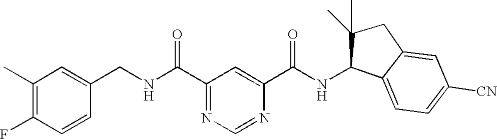 | 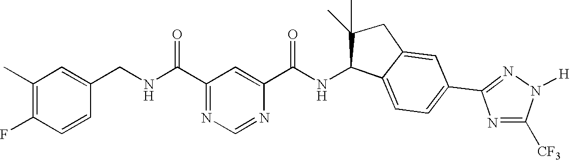 |
| 609 | 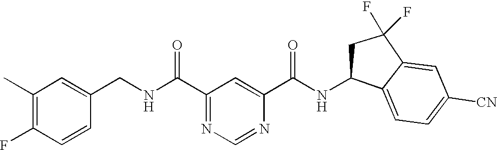 | 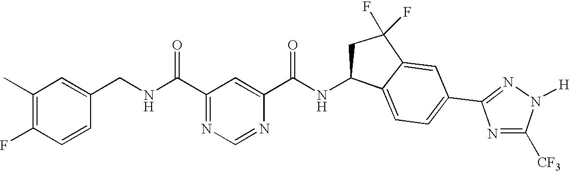 |
| 610 | 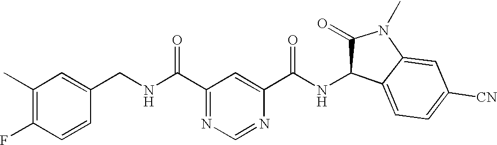 | 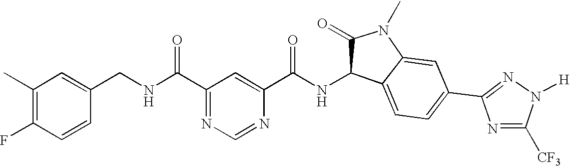 |
| 611 | 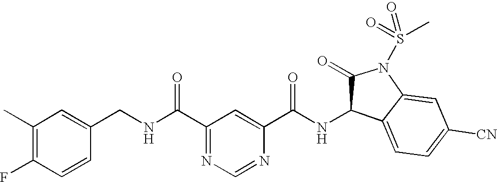 | 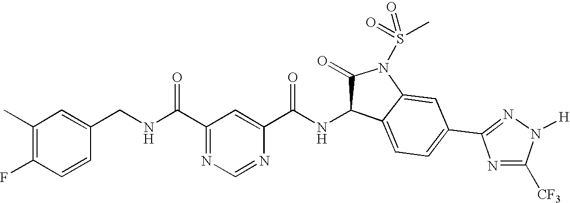 |
| 612 | 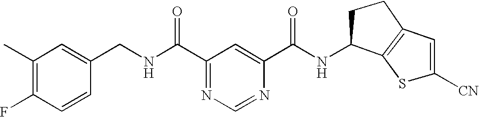 | 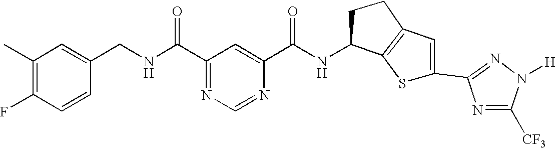 |
| 613 | 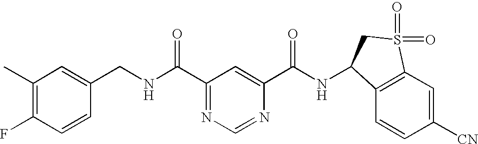 | 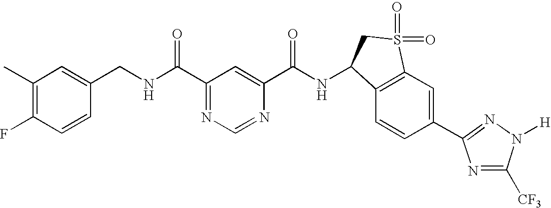 |
| 614 | 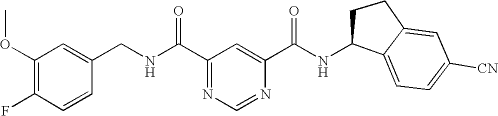 | 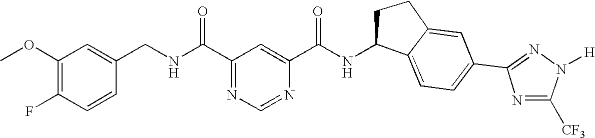 |
| 615 | 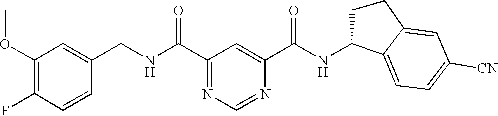 | 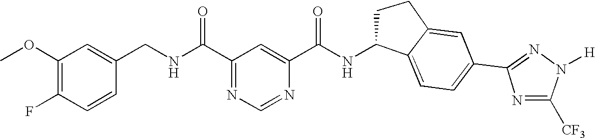 |
| 616 | 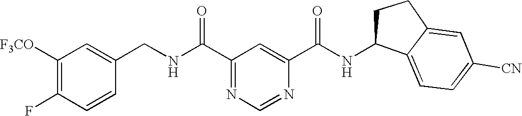 | 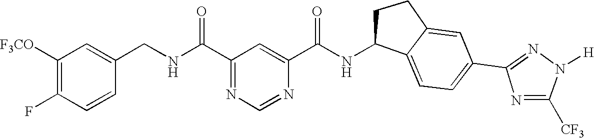 |
| 617 | 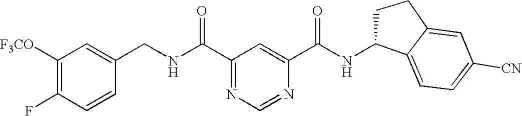 | 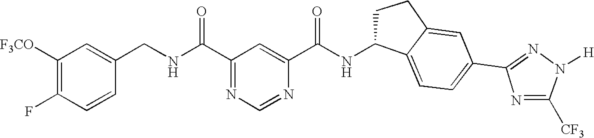 |
| 618 | 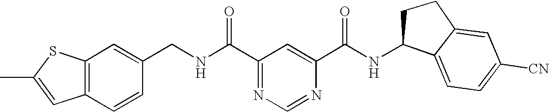 | 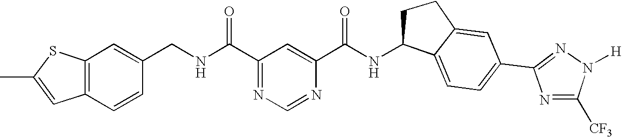 |
| 619 | 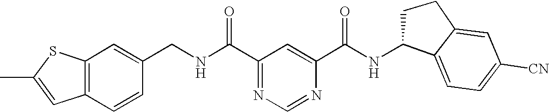 | 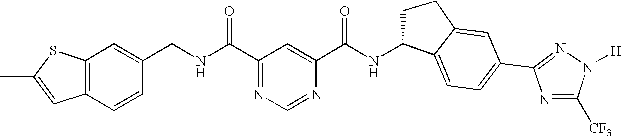 |
| 621 | 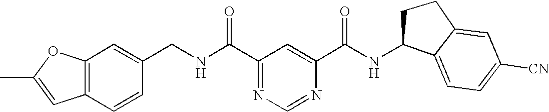 | 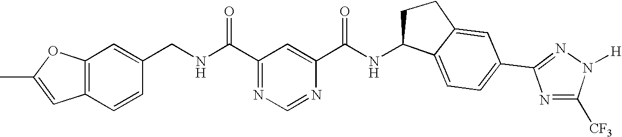 |
| 622 | 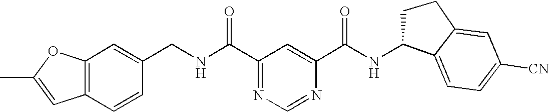 | 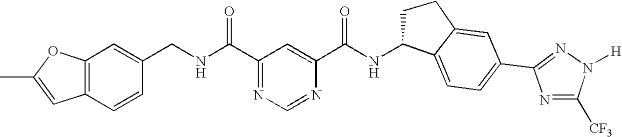 |
| 623 | 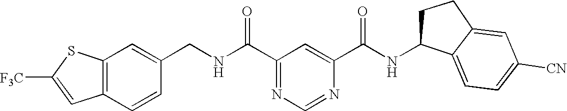 | 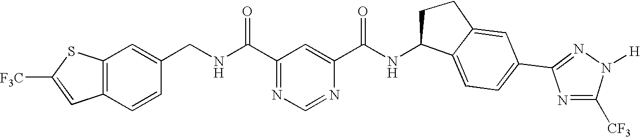 |
| 624 | 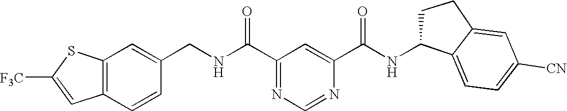 | 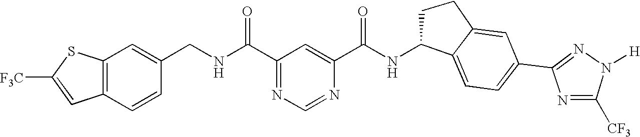 |
| 625 | 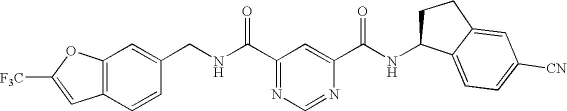 | 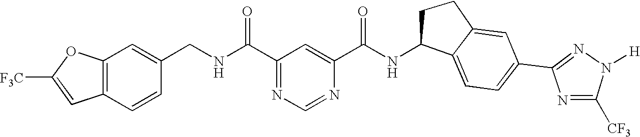 |
| 626 | 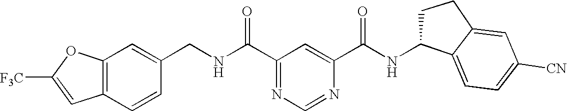 | 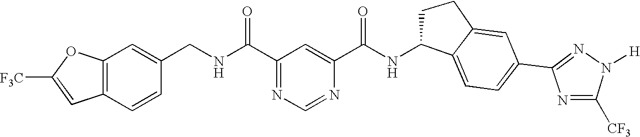 |
| 627 | 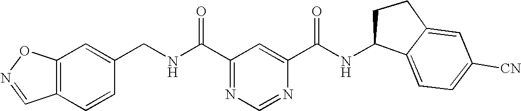 | 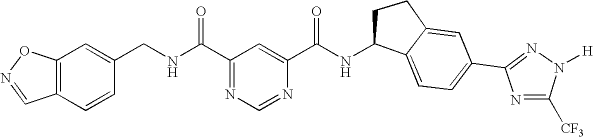 |
| 628 | 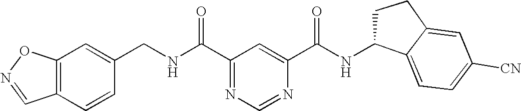 | 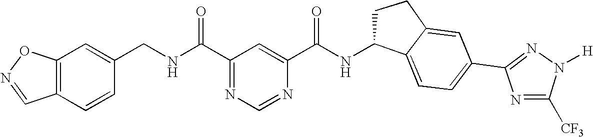 |
| 629 | 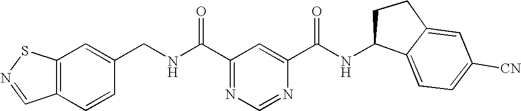 | 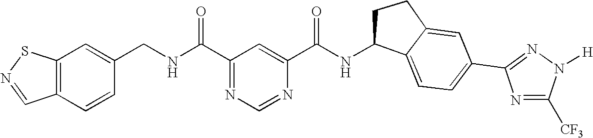 |
| 630 | 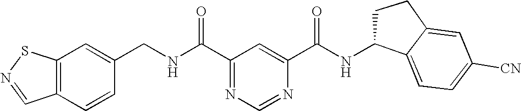 | 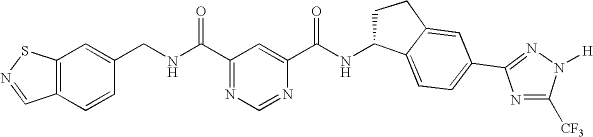 |

### Examples 701-730

If one were to heat the title compound from the list below according to similar procedures outlined in the Preparative Example 8 and Example 8, one would obtain the desired hydroxytriazole, as shown in Table 10 below.

TABLE 10

| Ex. # | Starting Material | Product |
|---|---|---|
| 701 | 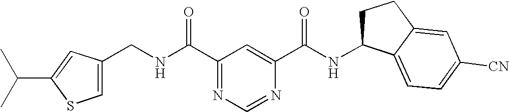 | 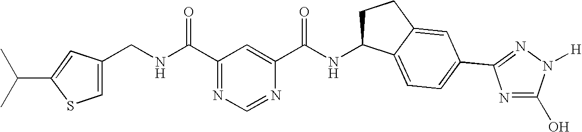 |
| 702 | 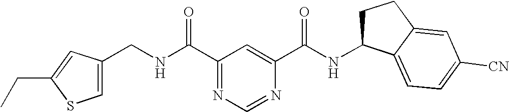 | 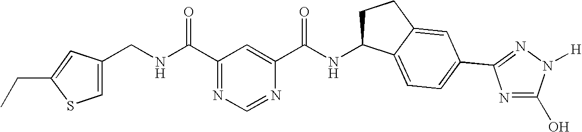 |
| 703 | 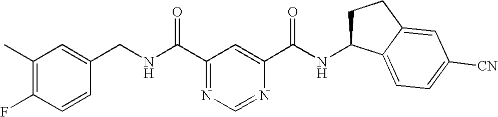 | 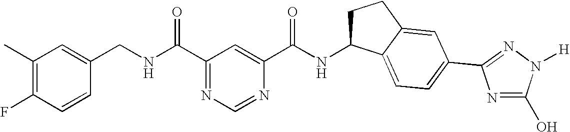 |
| 704 | 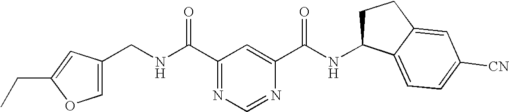 | 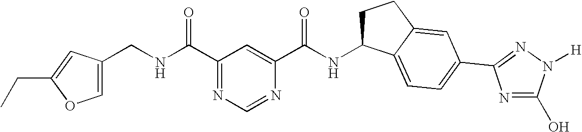 |
| 705 | 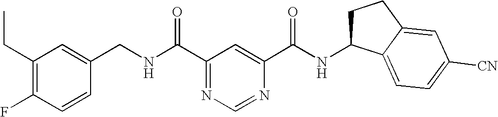 | 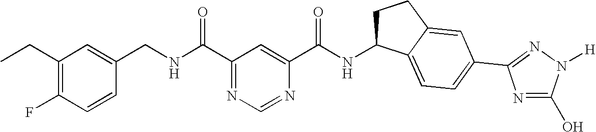 |
| 706 | 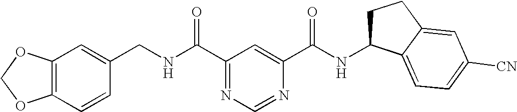 | 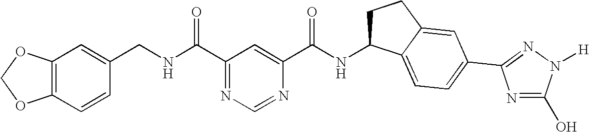 |
| 707 | 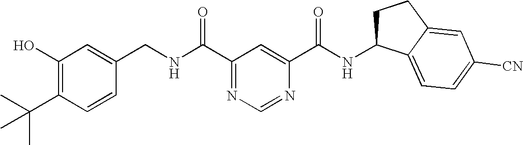 | 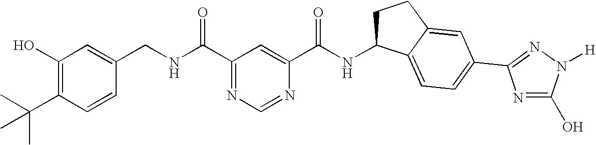 |
| 708 | 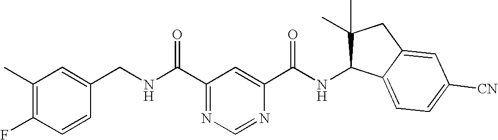 | 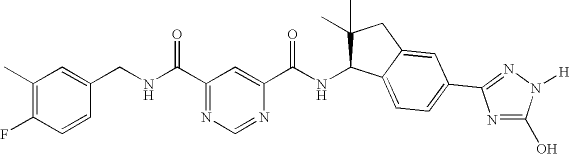 |
| 709 | 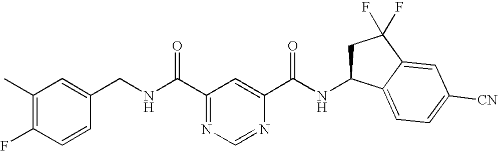 | 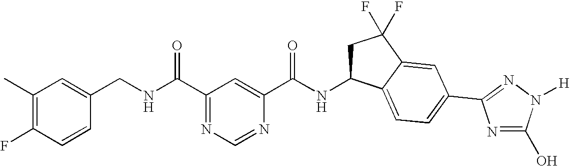 |
| 710 | 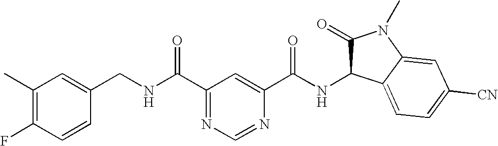 | 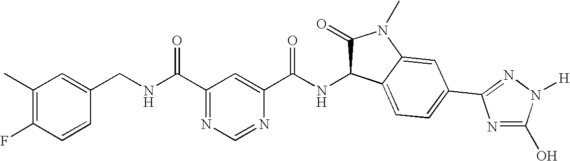 |
| 711 | 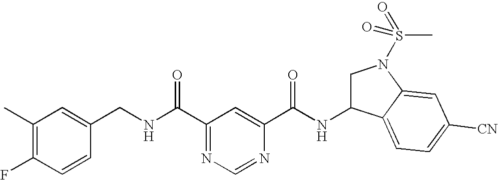 | 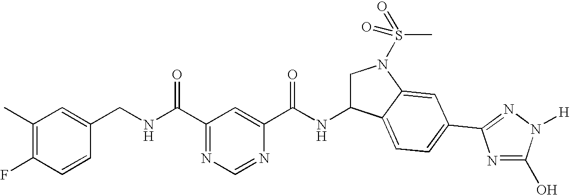 |
| 712 | 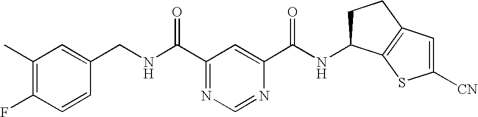 | 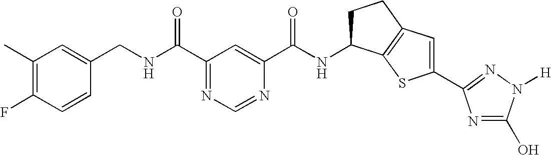 |
| 713 | 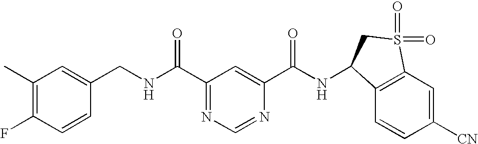 | 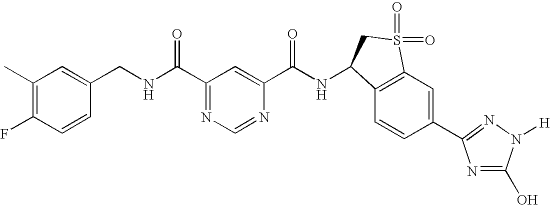 |
| 714 | 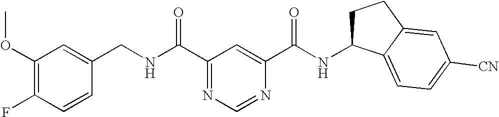 | 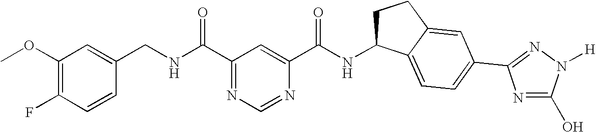 |
| 715 | 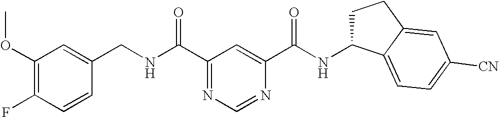 | 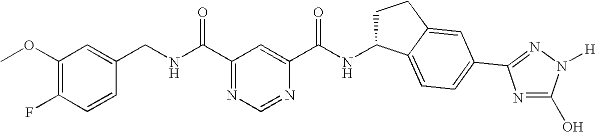 |
| 716 | 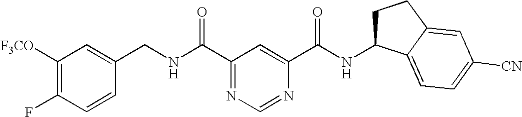 | 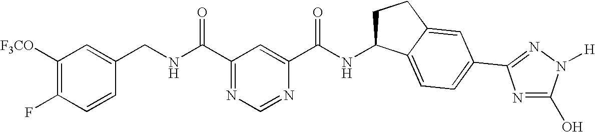 |
| 717 | 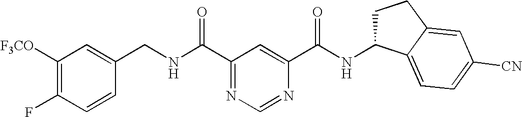 | 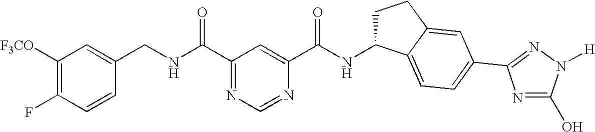 |
| 718 | 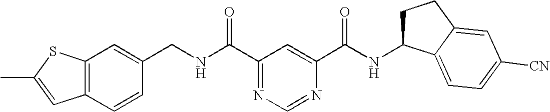 | 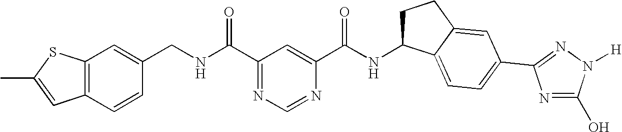 |
| 719 | 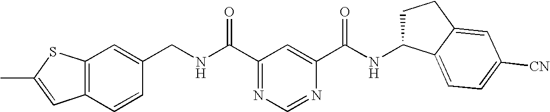 | 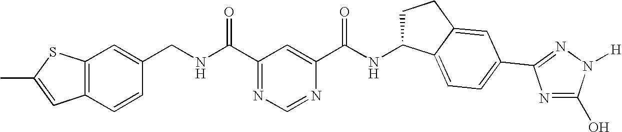 |
| 721 | 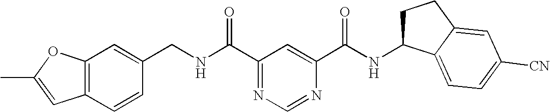 | 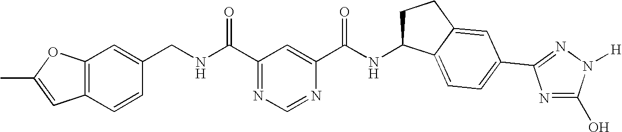 |
| 722 | 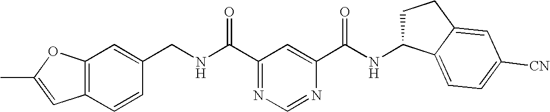 | 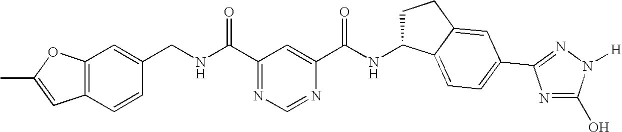 |
| 723 | 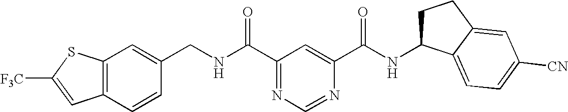 | 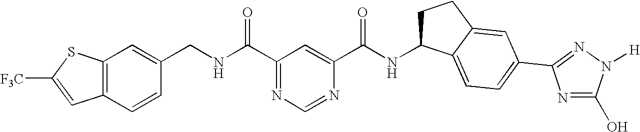 |
| 724 | 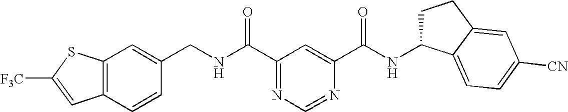 | 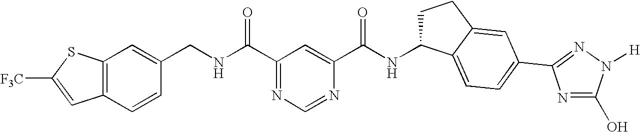 |
| 725 | 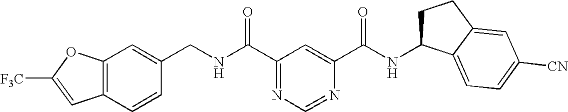 | 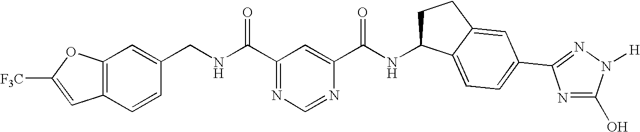 |
| 726 | 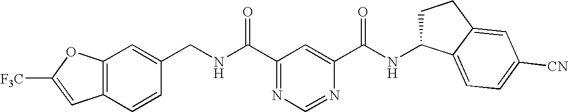 | 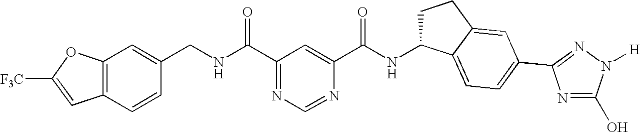 |
| 727 | 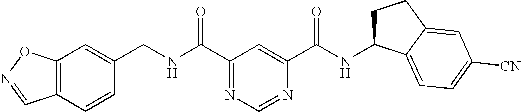 | 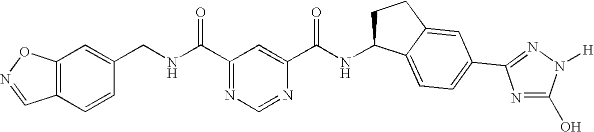 |
| 728 | 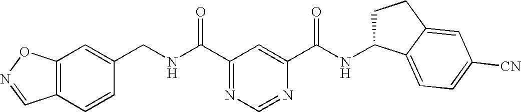 | 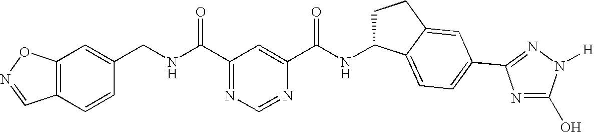 |
| 729 | 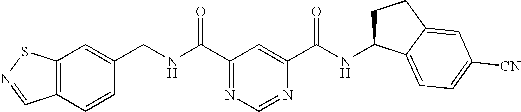 | 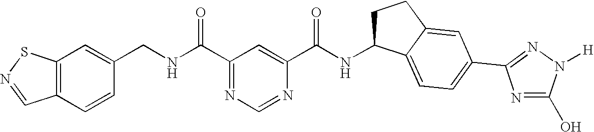 |
| 730 | 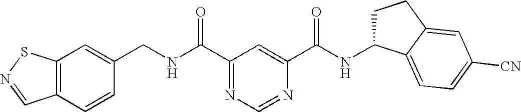 | 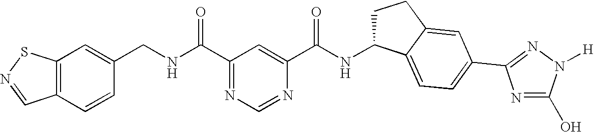 |

### Example 2300

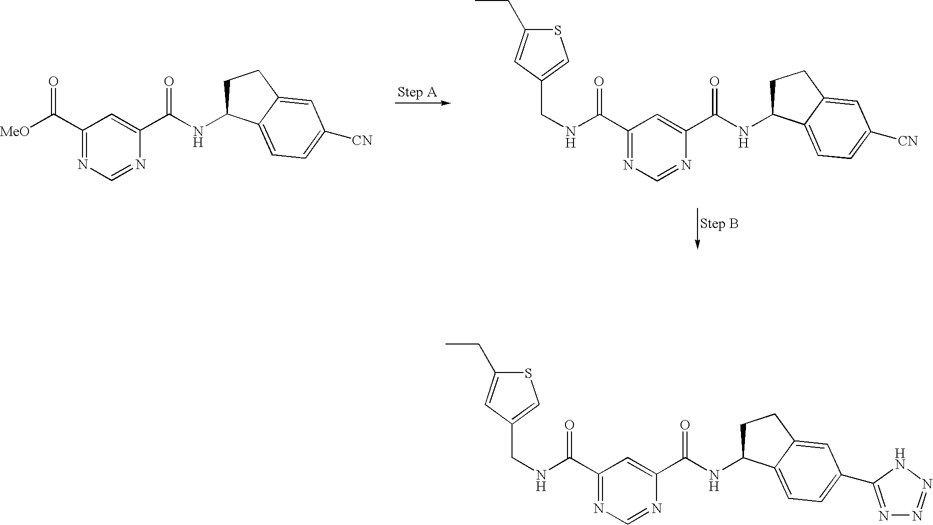 Step A

A solution of the intermediate from the Preparative Example 2004, Step B above (60 mg) in N,N-dimethylformamide (0.5 mL) was added to the title compound from the Preparative Example 2119, Step A and the mixture was stirred at 80° C. for 15 h, concentrated and then purified by column chromatography (silica, diethyl ether/dichloromethane, 3:7) to afford the intermediate (50 mg; 28%) as a colourless solid. [MH]⁺=420.

Step B

To the intermediate from Step A above (45 mg) in dry toluene (1.5 mL) was added SnO(Bu)₂ (10 mg) and azidotrimethylsilane (55 μL) and the mixture was heated (100 to 102° C.) under a nitrogen atmosphere for 18 h. The mixture was then concentrated and purified by preparative thin layer chromatography (silica, methanol/dichloromethane, 3:19) to afford the title compound (30 mg; 63%) as a foam. ¹H-NMR (DMSO) δ=1.25 (t, 3 H), 2.10-2.30 (m, 1 H), 2.75 (q, 2 H), 2.8-3.2 (m, 3 H), 4.12 (d, 2 H), 5.64 (q, 1 H), 6.76 (s, 1 H), 7.12 (s, 1 H), 7.20 (d, 1 H), 7.80 (d, 1 H), 7.97 (s, 1 H), 8.52 (s, 1 H), 9.35 (d, 1 H), 9.43 (s, 1 H), 9.64 (t, 1 H).

### Example 2301

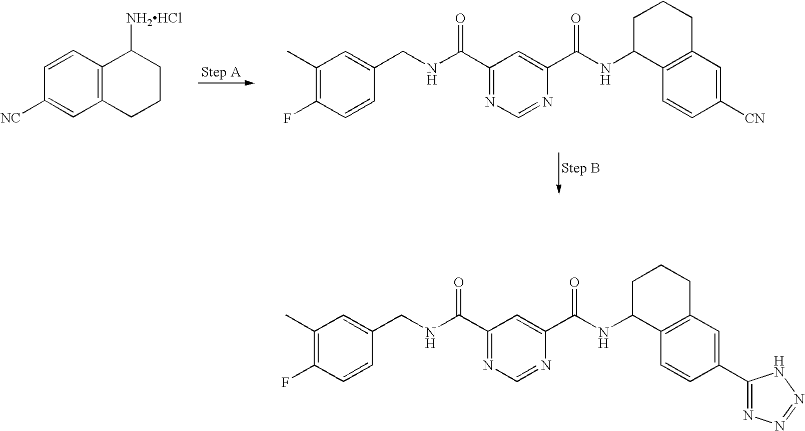 Step A

To a mixture of commercially available 6-cyano-1,2,3,4-tetrahydro-naphthalen-1-yl-ammonium chloride (49.6 mg), the title compound from the Preparative Example 2120, Step B (57.3 mg), bromotripyrrolidinophosphonium hexafluorophosphate (113 mg) in TRF (2 mL) was added triethylamine (61 μL). The mixture was allowed to stir at room temperature for 18 h. EtOAc (10 mL) and 1N aqueous hydrochloric acid (10 mL) were added. The aqueous layer was washed two times with EtOAc (10 mL). The combined organic layers were washed with a saturated aqueous solution of NaHCO₃ (10 mL), brine (10 mL), dried over MgSO₄, filtered and concentrated. The resulting residue was purified by silica gel chromatography (hexanes/ethyl acetate, 1:1) to afford the intermediate as an off-white solid (51 mg; 48%). [MH]⁺=444.

Step B

A mixture of the intermediate from Step A above (51 mg), dibutyltin oxide (7 mg), azidotrimethylsilane (30.5 μL) and toluene (1 mL) under an atmosphere of argon in a sealed vial was allowed to stir at 110° C. for 18 h. The reaction mixture was concentrated and purified by silica gel chromatography (CH₂Cl₂/MeOH, 9:1) to give title compound as an off-white solid (21 mg; 38%). [MH]⁺=486.

### Examples 2302-2309

Following a similar procedure as that described in Example 2301, Step A and Step B, except using the acid and amine indicated in Table 11 below, the following compounds were prepared.

TABLE 11

| Ex. # | Amine Acid | Product | Yield MS |
|---|---|---|---|
| 2302 | 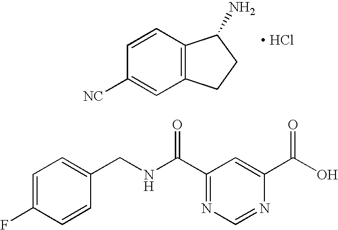 | 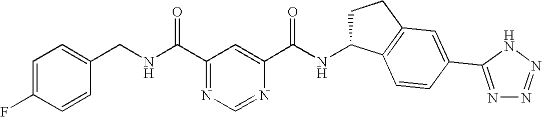 | 18% [MH]⁺ = 458 |
| 2303 | 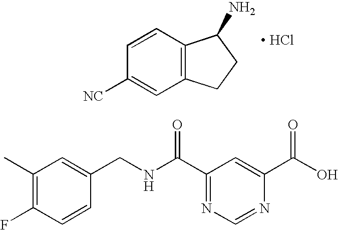 | 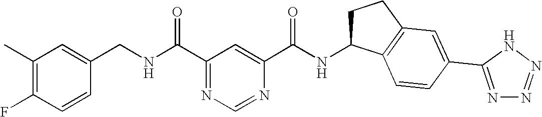 | 28% [MH]⁺ = 473 |
| 2304 | 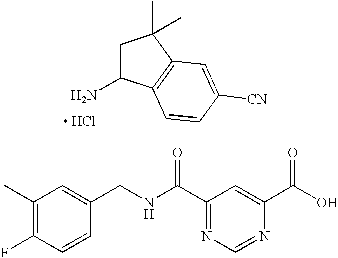 | 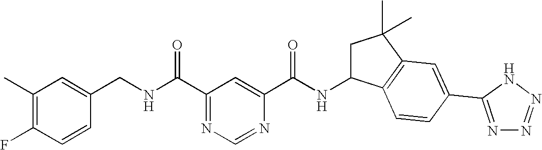 | 65% [MH]⁺ = 501 |
| 2305 | 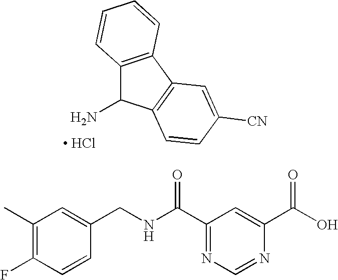 | 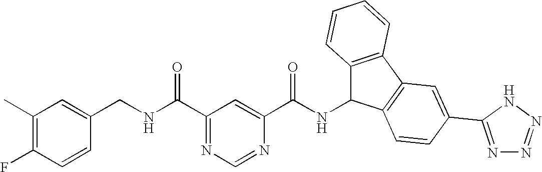 | 44% [MH]⁺ = 521 |
| 2306 | 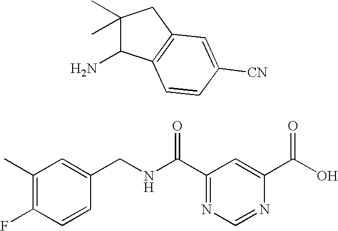 | 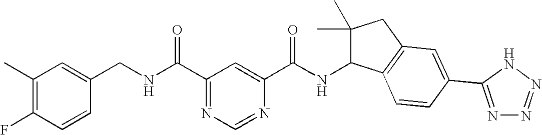 | 51% [MH]⁺ = 501 |
| 2307 | 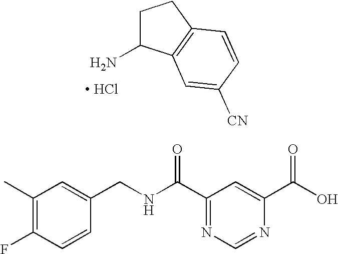 | 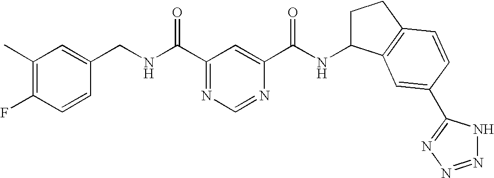 | 89% [MH]⁺ = 473 |
| 2308 | 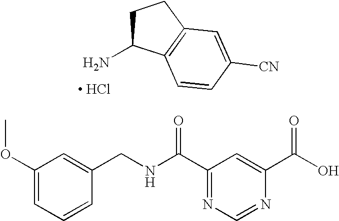 | 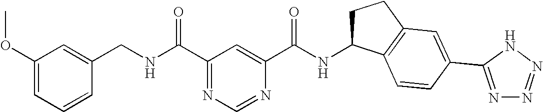 | 27% [MH]⁺ = 471 |
| 2309 | 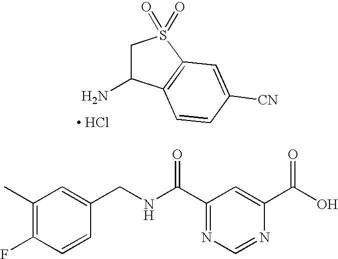 | 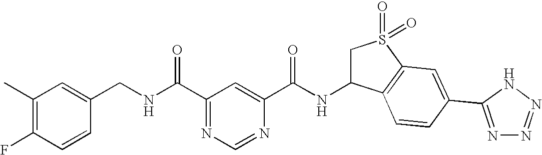 | 61% [MH]⁺ = 523 |

### Example 2310

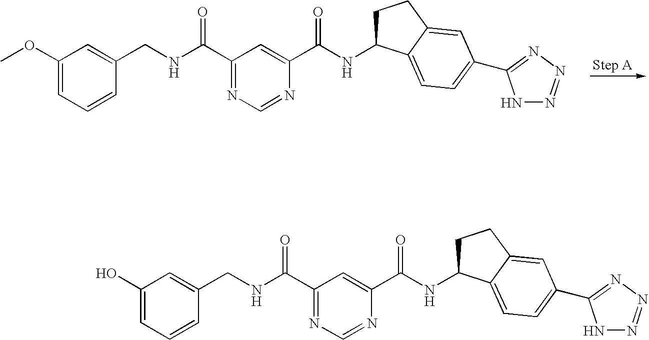 Step A

To a solution of the title compound from Example 2308 above (37 mg) in dry dichloromethane (390 μL) was added boron tribromide (1M in dichloromethane, 468 μL). The mixture was diluted with dichloromethane (2 mL) and stirred at room temperature for 2 h. Methanol (5 mL) was added and stirring at room temperature was continued for 1 h. The mixture was concentrated and purified by flash chromatography (silica, dichloromethane/methanol) to afford the title compound (35 mg; 99%). [MH]⁺=457.

### Example 2311

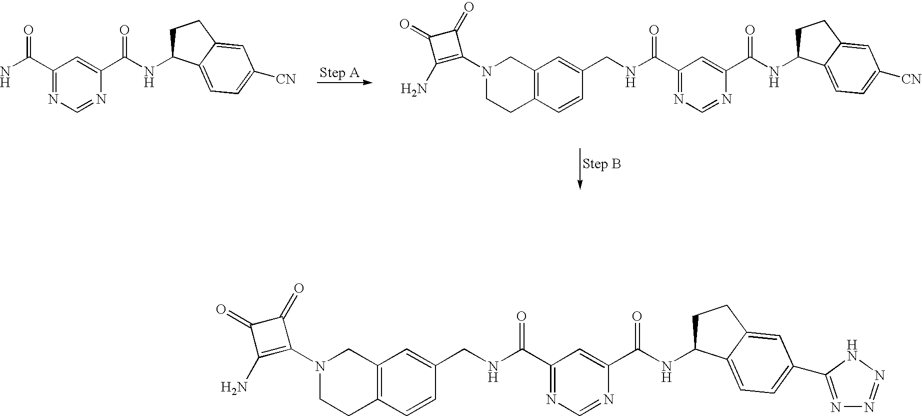 Step A

To a solution of the title compound from the Preparative Example 2119, Step B (51.5 mg) in DMF (3 mL), 1-ethyl-3-(3-dimethylaminopropyl)carbodiimide (42 mg), 1-hydroxy-benzotriazole (30 mg), the title compound from the Preparative Example 2043, Step C (58 mg) and N-methylmorpholine (100 μL) were added. After stirring at room temperature overnight, the mixture was concentrated to dryness. The residue was dissolved in ethyl acetate and washed with aqueous 1N hydrochloric acid, saturated NaHCO₃ and brine. The organic phase was separated, dried over MgSO₄, filtered and absorbed on silica. The residue was purified by column chromatography (silica, CH₂Cl₂/MeOH, 97:3 to 9:1) to afford the title compound as a yellow solid (74.6 mg; 82%). [MH]⁺=548.

Step B

A mixture of the intermediate from Step A above (74 mg), dibutyltin oxide (35 mg), azidotrimethylsilane (600 μL) and toluene (10 mL) and 1,2-dimethoxyethane (3 mL) under an atmosphere of argon in a sealed vial was allowed to stir at 110° C. for 2 d. To the reaction mixture was added methanol and the solution was absorbed on silica. Purification by silica gel chromatography (CH₂Cl₂/MeOH, 9:1 to 85:15) furnished the title compound as an off-white solid (17.8 mg; 22%). [MH]⁺=591.

### Examples 2312-2327

If one were to follow a similar procedure as that described in Example 2311, Step A and Step B, except using the amine from the Preparative Examples indicated in Table 12 below, the following title compounds would be obtained.

TABLE 12

| Ex. # | Amine | Product |
|---|---|---|
| 2312 | 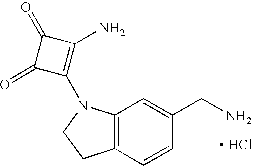 | 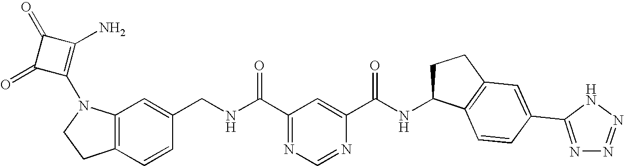 |
| 2313 | 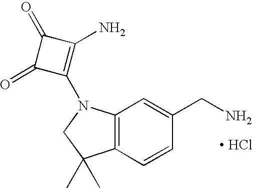 | 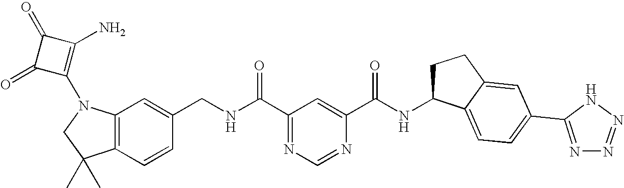 |
| 2314 | 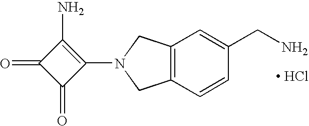 | 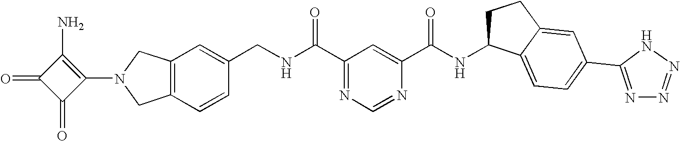 |
| 2315 | 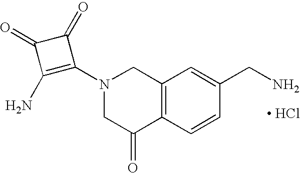 | 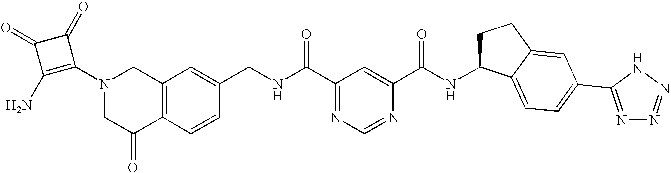 |
| 2316 | 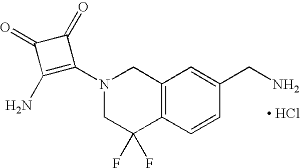 | 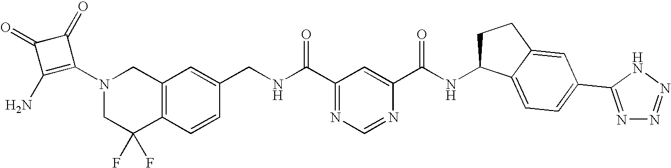 |
| 2317 | 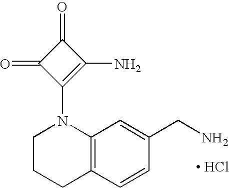 | 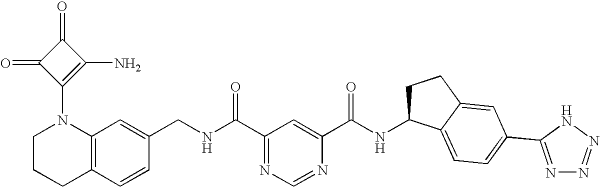 |
| 2318 | 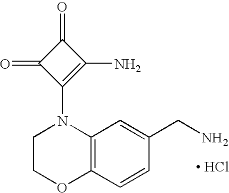 | 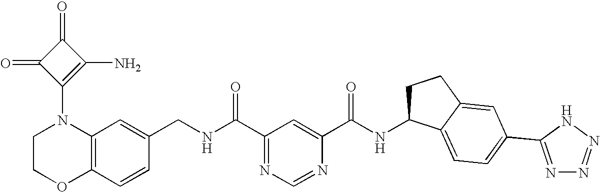 |
| 2319 | 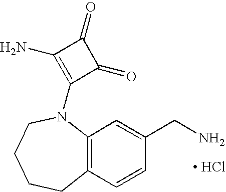 | 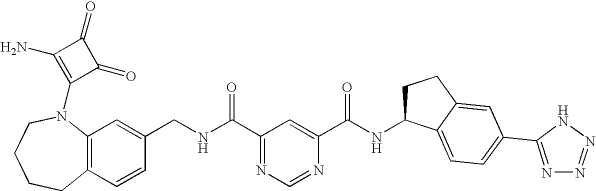 |
| 2320 | 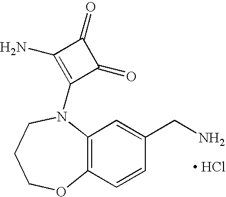 | 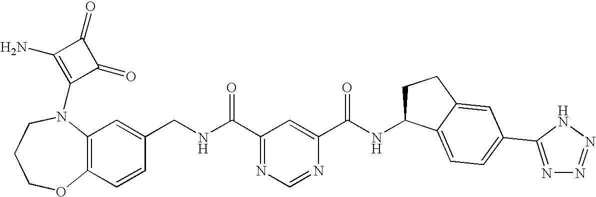 |
| 2321 | 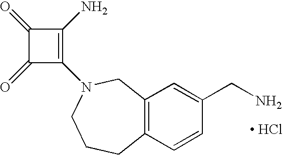 | 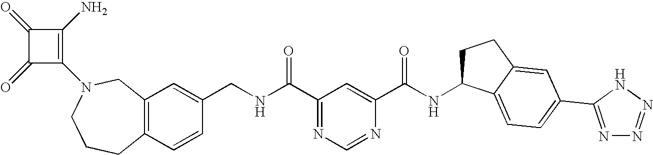 |
| 2322 | 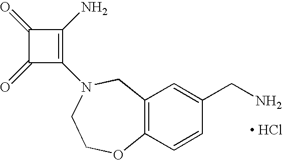 | 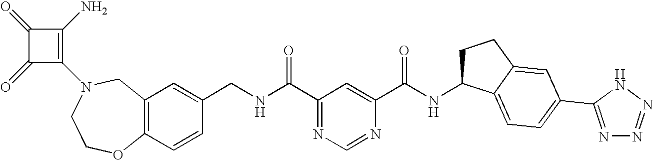 |
| 2323 | 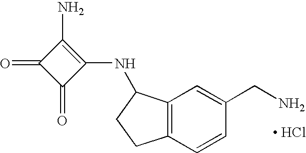 | 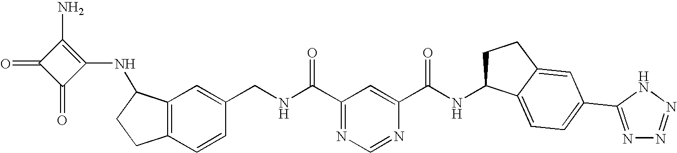 |
| 2324 | 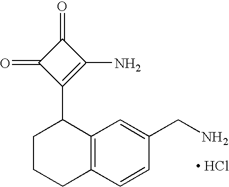 | 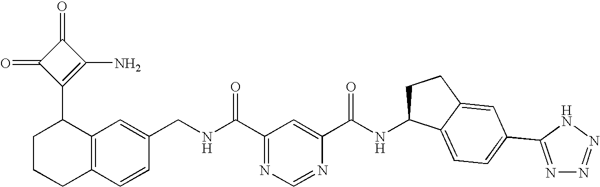 |
| 2325 | 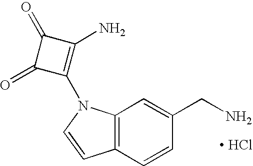 | 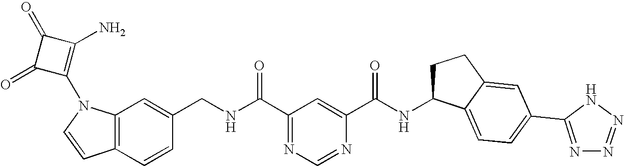 |
| 2326 | 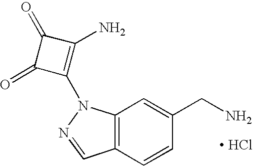 | 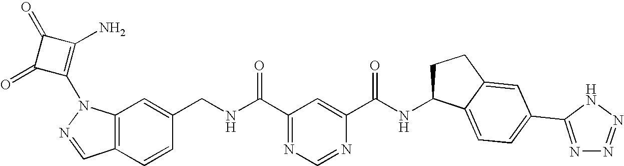 |
| 2327 | 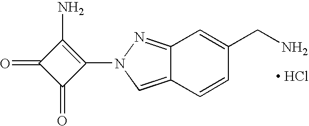 | 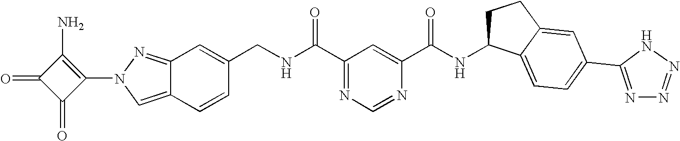 |

### Examples 2328-2329

Following a similar procedure as that described in Example 2311, Step A and Step B, except using the acid and amine indicated in Table 13 below, the following compounds were prepared.

TABLE 13

| Ex. # | Amine Acid | Product | Yield MS |
|---|---|---|---|
| 2328 | 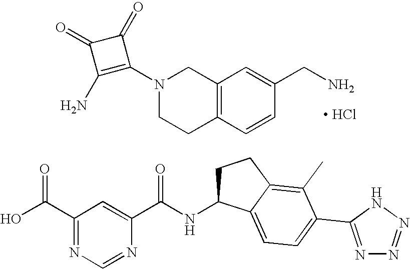 | 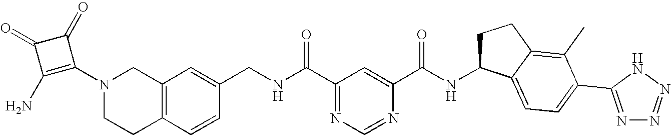 | 18% [MH]⁺ = 605 |
| 2329 | 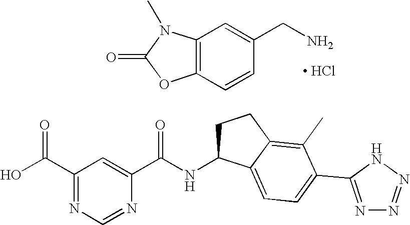 | 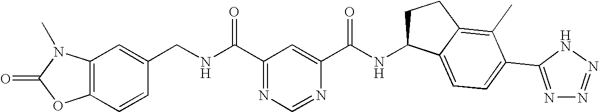 | 74% [MH]⁺ = 526 |

### Example 2400

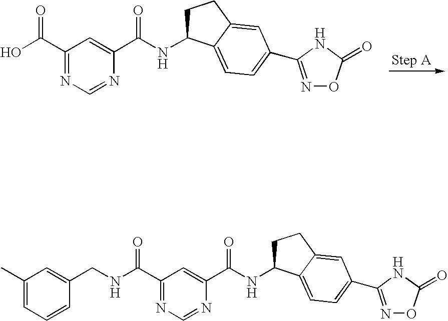 Step A

To a solution of the title compound from the Preparative Example 2117, Step C (28 mg) in DMF (1 mL), 1-ethyl-3-(3-dimethylaminopropyl)carbodiimide (16 mg), 1-hydroxy-benzotriazole (11.3 mg), 3-methyl-benzylamine hydrochloride (9.6 mg) and N-methylmorpholine (9.3 μL) were added. After stirring at room temperature for 12 h, the mixture was concentrated to dryness. The residue was dissolved in ethyl acetate and washed with saturated NaHCO₃, aqueous 1N hydrochloric acid and brine. The organic phase was separated, dried over MgSO₄, filtered and concentrated. The residue was purified by column chromatography (silica, CH₂Cl₂/MeOH, 95:5) to afford the title compound as colourless solid (29 mg; 88%). [MH]⁺=471.

### Examples 2401-2451

Following a similar procedure as that described in Example 2400, except using the compounds commercially available or from the Preparative Examples indicated in Table 14 below, the following compounds were prepared.

TABLE 14

| Ex. # | Amine | Product | Yield MS |
|---|---|---|---|
| 2401 | 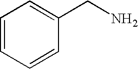 | 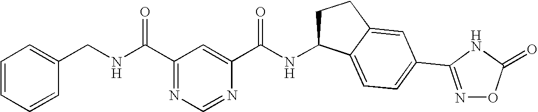 | 77% [MH]⁺ = 457 |
| 2402 | 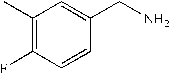 | 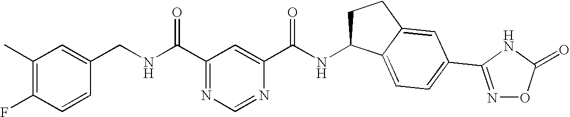 | 67% [MH]⁺ = 489 |
| 2403 | 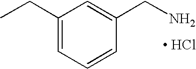 | 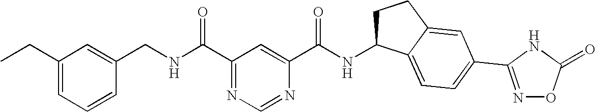 | 88% [MH]⁺ = 485 |
| 2404 | 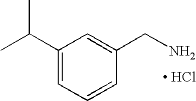 | 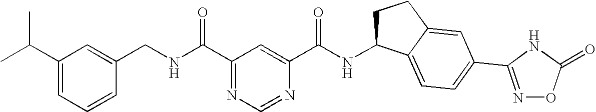 | 97% [MH]⁺ = 499 |
| 2405 | 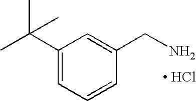 | 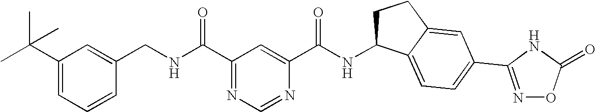 | 83% [MH]⁺ = 513 |
| 2406 | 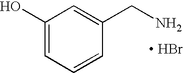 | 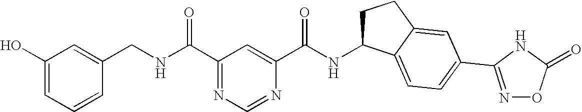 | 95% [MH]⁺ = 473 |
| 2407 | 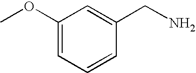 | 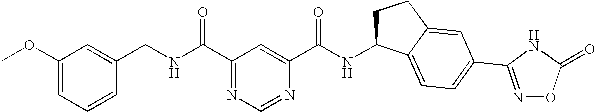 | 74% [MH]⁺ = 487 |
| 2408 | 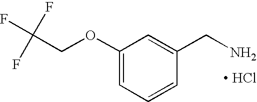 | 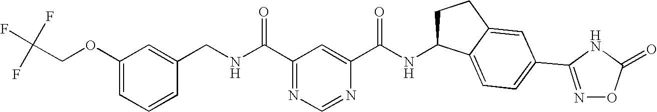 | 77% [MH]⁺ = 555 |
| 2409 | 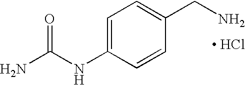 | 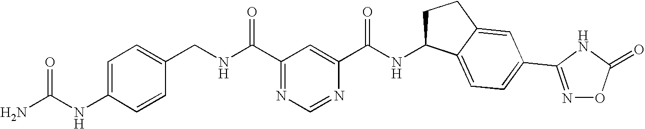 | 55% [MH]⁺ = 515 |
| 2410 | 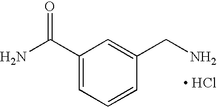 | 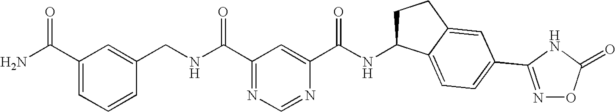 | 63% [MH]⁺ = 500 |
| 2411 | 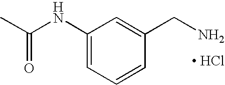 | 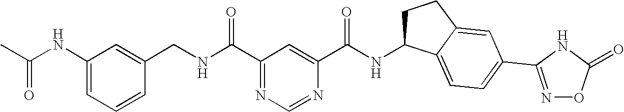 | 57% [MH]⁺ = 514 |
| 2412 | 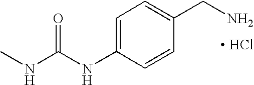 | 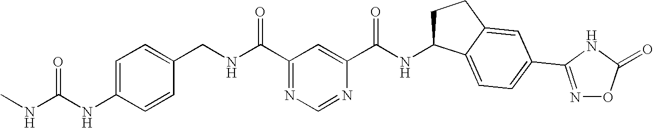 | 54% [MH]⁺ = 529 |
| 2413 | 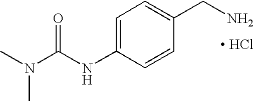 | 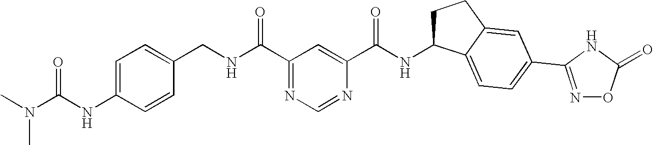 | 10% [MH]⁺ = 543 |
| 2414 | 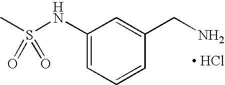 | 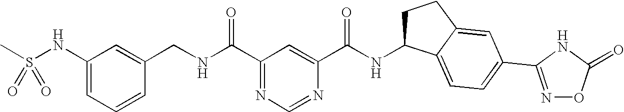 | 32% [MH]⁺ = 550 |
| 2415 | 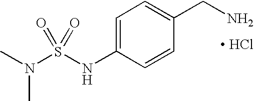 | 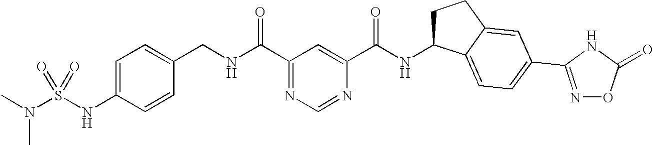 | 36% [MH]⁺ = 579 |
| 2416 | 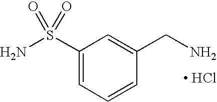 | 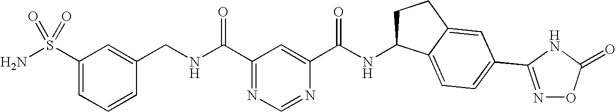 | 80% [MH]⁺ = 536 |
| 2417 | 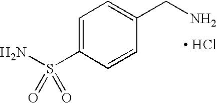 | 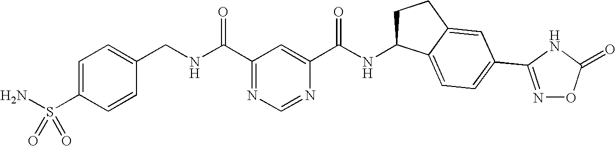 | 36% [MH]⁺ = 536 |
| 2418 | 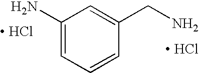 | 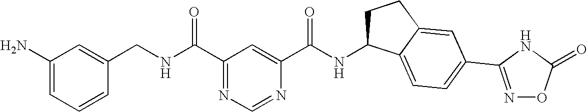 | 68% [MH]⁺ = 472 |
| 2419 | 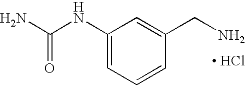 | 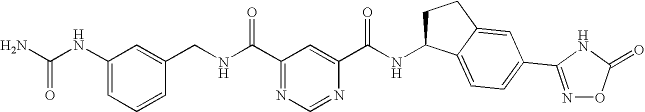 | 62% [MH]⁺ = 515 |
| 2420 | 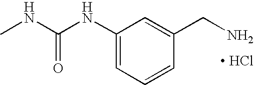 | 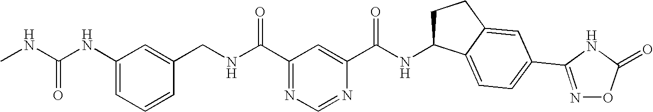 | 85% [MH]⁺ = 529 |
| 2421 | 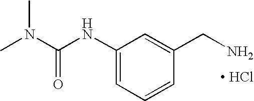 | 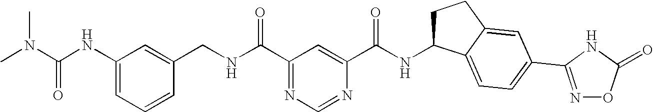 | 94% [MH]⁺ = 543 |
| 2422 | 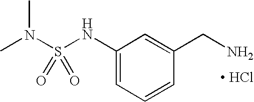 | 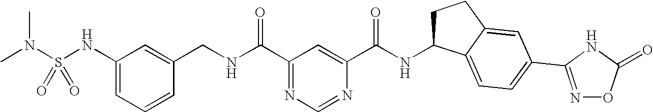 | 86% [MH]⁺ = 579 |
| 2423 | 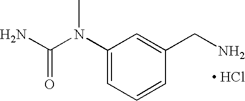 | 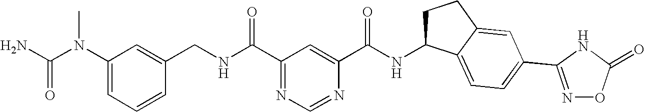 | 68% [MH]⁺ = 529 |
| 2424 | 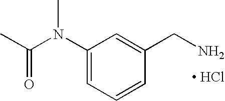 | 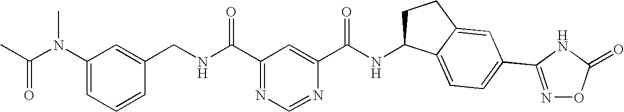 | 57% [MH]⁺ = 528 |
| 2425 | 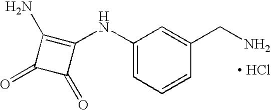 | 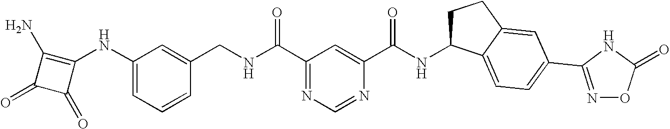 | 60% [MH]⁺ = 567 |
| 2426 | 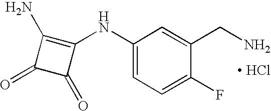 | 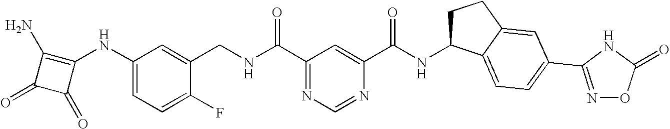 | 48% [MH]⁺ = 585 |
| 2427 | 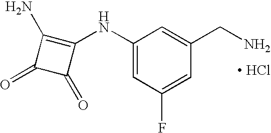 | 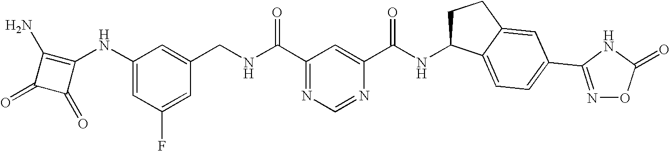 | 65% [MH]⁺ = 585 |
| 2428 | 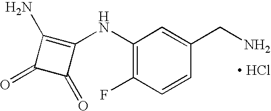 | 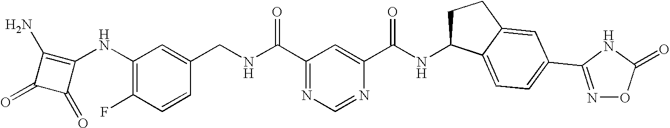 | 48% [MH]⁺ = 585 |
| 2429 | 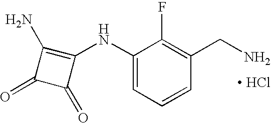 | 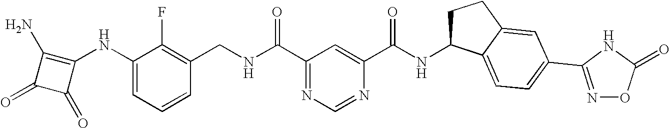 | 41% [MH]⁺ = 585 |
| 2430 | 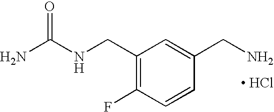 | 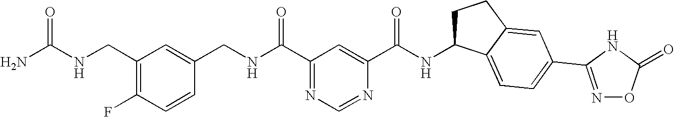 | 56% [MH]⁺ = 547 |
| 2431 | 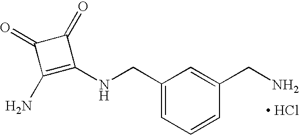 | 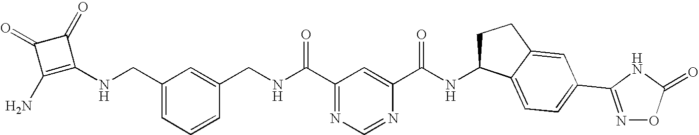 | 32% [MH]⁺ = 581 |
| 2432 | 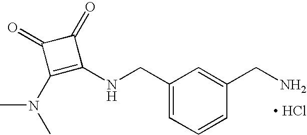 | 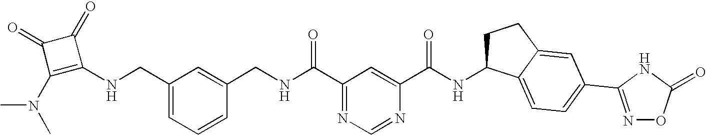 | 47% [MH]⁺ = 569 |
| 2433 | 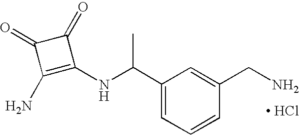 | 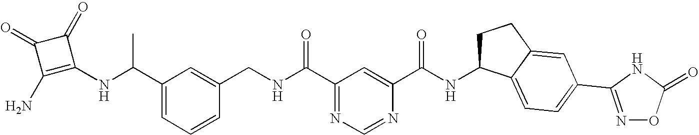 | 44% [MH]⁺ = 595 |
| 2434 | 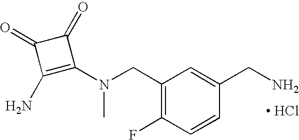 | 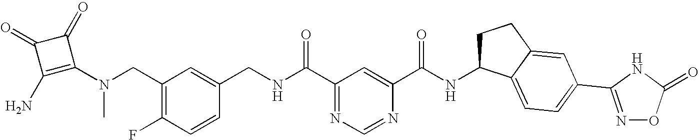 | 65% [MH]⁺ = 613 |
| 2435 | 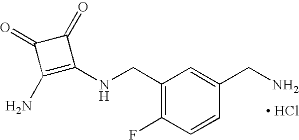 | 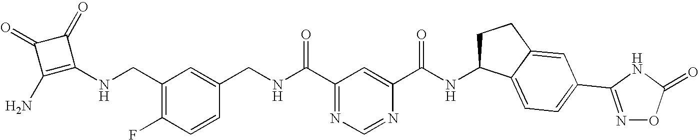 | 35% [MH]⁺ = 599 |
| 2436 | 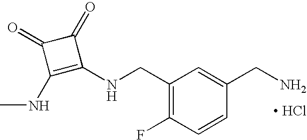 | 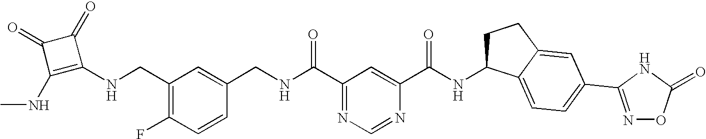 | 31% [MH]⁺ = 613 |
| 2437 | 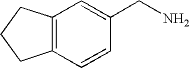 | 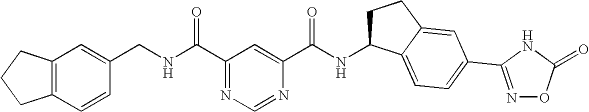 | 80% [MH]⁺ = 497 |
| 2438 | 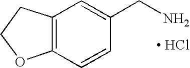 | 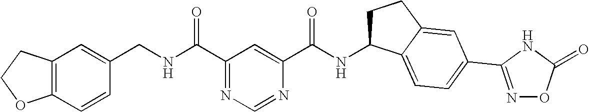 | 96% [MH]⁺ = 499 |
| 2439 | 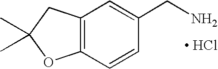 | 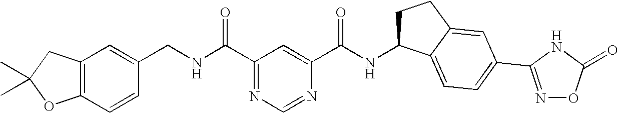 | 60% [MH]⁺ = 527 |
| 2440 | 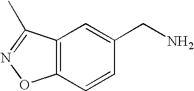 | 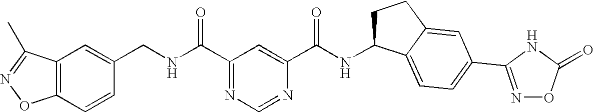 | 73% [MH]⁺ = 512 |
| 2441 | 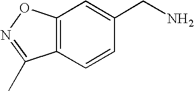 | 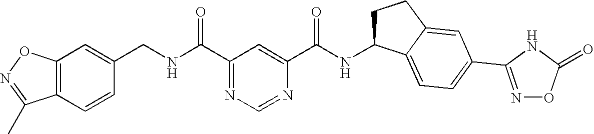 | 87% [MH]⁺ = 512 |
| 2442 | 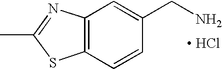 | 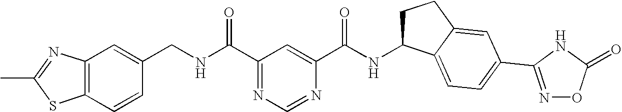 | 85% [MH]⁺ = 528 |
| 2443 | 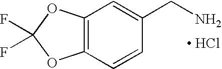 | 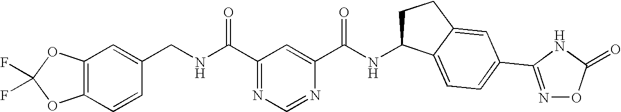 | 97% [MH]⁺ = 537 |
| 2444 | 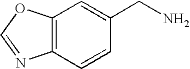 | 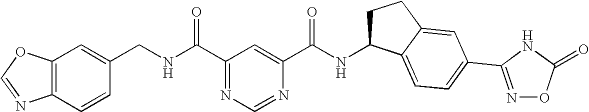 | 30% [MH]⁺ = 498 |
| 2445 | 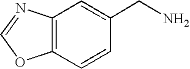 | 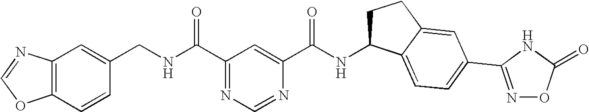 | 18% [MH]⁺ = 498 |
| 2446 | 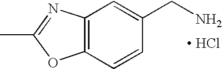 | 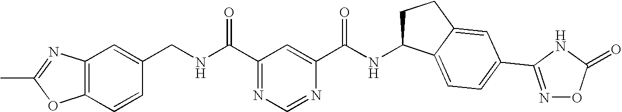 | 55% [MH]⁺ = 512 |
| 2447 | 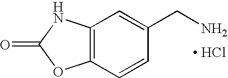 | 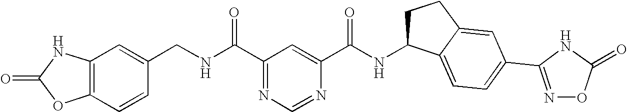 | 86% [MH]⁺ = 514 |
| 2448 | 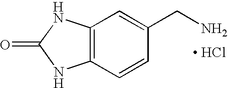 | 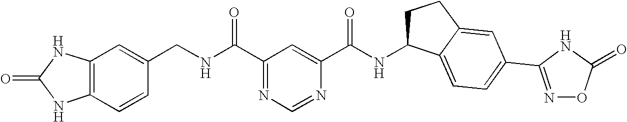 | 59% [MH]⁺ = 513 |
| 2449 | 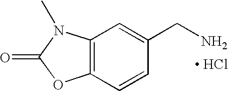 | 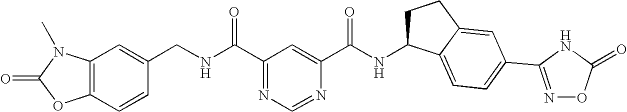 | 76% [MH]⁺ = 528 |
| 2450 | 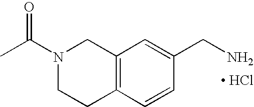 | 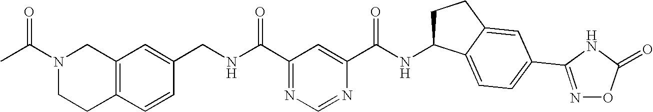 | 25% [MH]⁺ = 554 |
| 2451 | 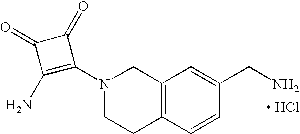 | 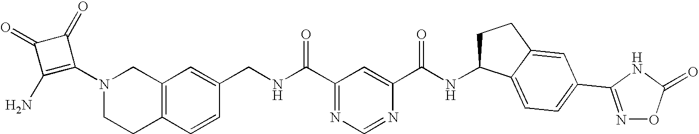 | 34% [MH]⁺ = 607 |

### Example 2452

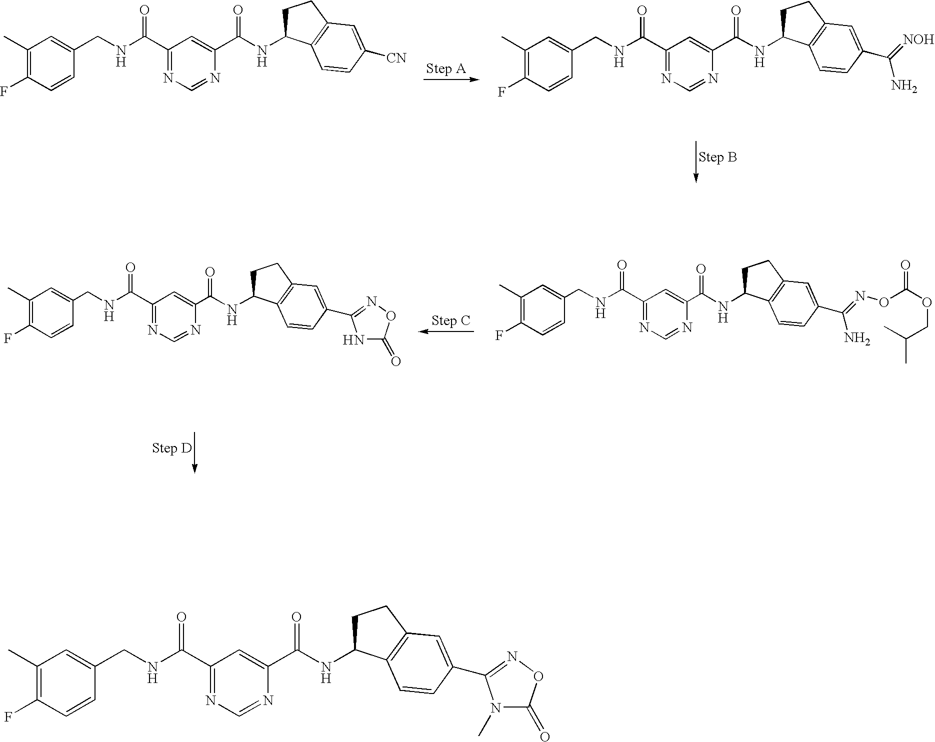 Step A

The intermediate from the Example 2303 (41 mg, 0.1 mmol) was refluxed with hydroxylamine (69 mg hydrochloride salt neutralized with grounded potassium hydroxide in ethanol) in ethanol (3 mL) overnight. The reaction mixture was concentrated to dryness to give the intermediate as a colourless solid, which was utilized in next step without further purification. [MH]⁺=463.

Step B

The compound from Step A above was dissolved in N,N-dimethylformamide (1 mL) and cooled to 0° C. in an ice bath. Pyridine (9 μL, 0.11 mmol) was added followed by the addition of isobutyl chloroformate (13 μL, 0.105 mmol). The reaction was kept at the same temperature for 30 min and then concentrated to dryness to give the intermediate as brown oil. [MH]⁺=563.

Step C

To the compound from Step B above was added chlorobenzene (3 mL) and refluxed for 3 h. The reaction mixture was concentrated to dryness. The crude material was purified by column chromatography to furnish the intermediate (28 mg; 60% for 3 steps) as an off-white solid. [MH]⁺=489.

Step D

To the compound from Step C above (26 mg, 53 μmol) in a benzene and methanol mixture (1.2 mL, 3:1) was added trimethylsilyldiazomethane (2M solution in diethyl ether, 29 μL) and stirred for 1 h. The solution was concentrated in vaccuo. The brown solid was purified by silica gel chromatography to give the title compound (24 mg; 90%) as an off-white solid. [MNa]⁺=525.

### Example 2453

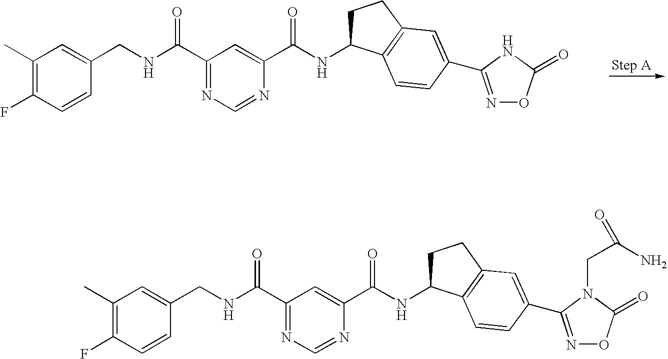 Step A

The intermediate from the Example 2452, Step C (40 mg) was dissolved in acetone (1 mL), and potassium carbonate (12 mg) and 2-bromoacetamide (12 mg) were added. The reaction was stirred for several hours at room temperature, then heated to 55° C. After 4 h, more 2-bromoacetamide (12 mg) was added and the reaction was heated overnight. Volatiles were removed under reduced pressure and the residue taken up in dichloromethane and water. The organic layer was concentrated under vacuum and purified by column chromatography (5% methanol in dichloromethane) to give the title compound as a colourless solid (33 mg). [MH]⁺=546.

### Example 2454

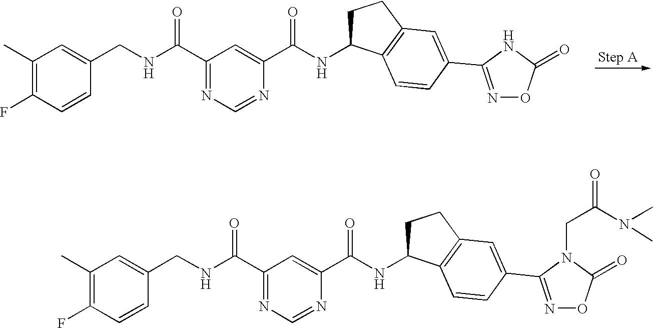 Step A

The intermediate from the Example 2452, Step C (35 mg) was dissolved in acetone (0.75 mL) and potassium carbonate (9 mg) and 2-chlorodimethylacetamide (11 mg) were added. The reaction was heated at 55° C. overnight. Sodium iodide (10 mg) was added and the reaction was heated overnight. Volatiles were removed under reduced pressure and the residue dissolved in aqueous ammonium chloride and dichloromethane. Purification of the organic residue by column chromatography (5% methanol in dichloromethane) yielded the title compound (40 mg). [MH]⁺=574.

### Example 2455

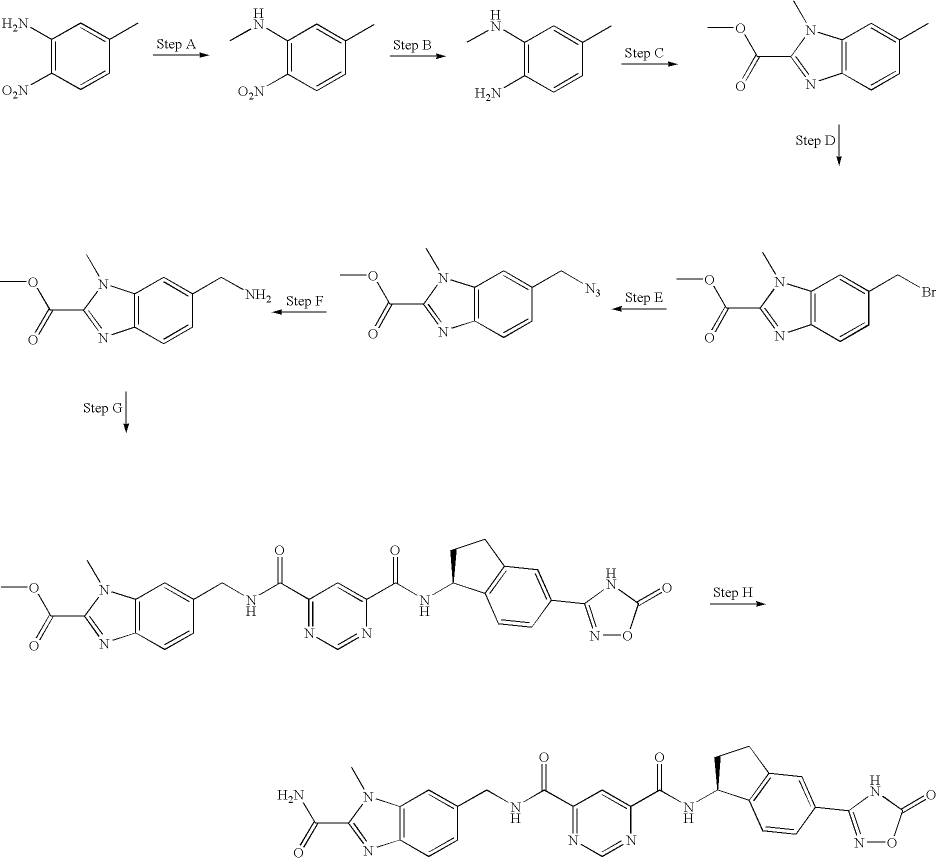 Step A

To a solution of commercially available 5-methyl-2-nitro-phenylamine (5.00 g) in DMF (100 mL) was added sodium hydride (790 mg) at 0° C. and the mixture was stirred for 10 min at this temperature. Then methyl iodide (18.7 g) was added over a period of 30 min and the mixture was stirred for 1 h at 0° C. and 1 h at room temperature. The mixture was concentrated and the residue dissolved in ethyl acetate. The organic layer was washed with water and brine, dried (MgSO₄), concentrated and purified by crystallisation from ethanol to afford the title compound (2.83 g; 52%) as red needles. ¹H-NMR (DMSO) δ=2.25 (s, 3 H), 2.92 (d, 3H), 6.42 (d, 1 H), 6.75 (s, 1 H), 7.90 (d, 1 H), 8.10 (br s, 1 H).

Step B

A mixture of the title compound from Step A above (2.83 g) and palladium on charcoal (10 wt %, 1.5 g) in ethanol (20 mL) was stirred at room temperature for 16 h. The mixture was filtered through a plug of celite® to afford the title compound (2.02 g; 87%) as an oil. ¹H-NMR (DMSO) δ=2.10 (s, 3 H), 2.65 (s, 3 H), 4.32 (br s, 3 H), 6.20 (s, 1 H), 6.22 (d, 1 H), 6.40 (d, 1 H).

Step C

A solution of the title compound from Step B above (2.00 g) in trimethoxy-acetic acid methyl ester, prepared as described by W. Kantlehner et al. (Liebigs Ann. Chem. 1980, 1448-1454), was heated to 100° C. for 16 h. The mixture was cooled down to 50° C. and diethyl ether was added. The mixture was kept for 1 h at 0° C. and decanted. The residue was concentrated and purified by column chromatography (silica, chloroform/MeOH, 98:2). Crystallisation from Et₂O/EtOH afforded the title compound (759 mg; 25%) as a solid. [MH]⁺=205.

Step D

A solution of the title compound from Step C above (309 mg), NBS (351 mg) and AIBN (10 mg) in tetrachloromethane was refluxed for 4 h. After the precipitate was filtered off, the organic layer was concentrated and purified by column chromatography (silica, chloroform/MeOH, 98:2) to afford the title compound (100 mg; 23%). [MH]⁺=283.

Step E

A mixture of the title compound from Step D above (1.00 g) and sodium azide (720 mg) in DMF (3 mL) was stirred at room temperature for 16 h. The mixture was concentrated and the residue dissolved in ethyl acetate. The organic layer was concentrated and purified by column chromatography (silica, cyclohexane/EtOAc, 6:4) to afford the title compound (90 mg; 99%) as a colourless solid. [MH]⁺=246.

Step F

A solution of the title compound from Step E above (49 mg) and triphenylphosphine (68 mg) in tetrahydrofurane (2 mL) was stirred at room temperature for 16 h. Then water (1 mL) was added and the mixture was stirred for 5 h at 50° C. The mixture was concentrated and purified by column chromatography (silica, chloroform/MeOH, 80:20) to afford the title compound (23 mg; 50%) as a colourless solid. [MH]⁺=220.

Step G

A solution of the title compound from Step F above (23 mg), the title compound from the Preparative Example 2117, Step C (50 mg), 1-(3-dimethylaminopropyl)-3-carbodiimide hydrochloride (26 mg), 1-hydroxy-benzotriazole (18 mg), DMAP (1 mg) and DIPEA (18 mg) in DMF (2 mL) was stirred at room temperature for 3 d. The mixture was concentrated and the residue dissolved in ethyl acetate. The organic layer was washed with 0.O1M hydrochloric acid, 0.01 mM KOH, dried (MgSO₄), and concentrated to afford the title compound (40 mg; 68%) as a solid. [MH]⁺=569.

Step H

A mixture of the title compound from Step G above (22 mg) in 30% aqueous ammonia (40 mL) was heated to 100° C. in a sealed pressure tube for 16 h. The mixture was concentrated and purified by preparative thin layer chromatography (chloroform/MeOH 90:10) to afford the title compound (2 mg; 10%). [MH]⁺=554.

### Examples 2456-2471

If one were to follow a similar procedure as that described in Example 2400 except using the amine from the Preparative Examples indicated in Table 15 below, the following title compounds would be obtained.

TABLE 15

| Ex. # | Amine | Product |
|---|---|---|
| 2456 | 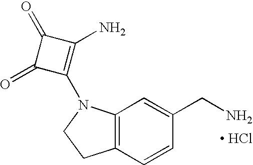 | 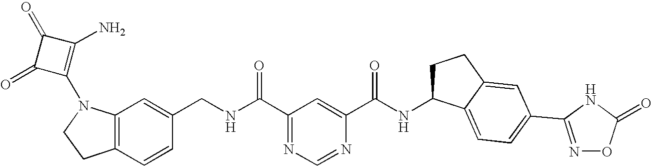 |
| 2457 | 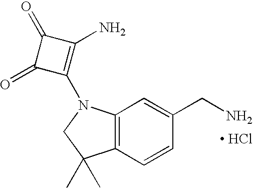 | 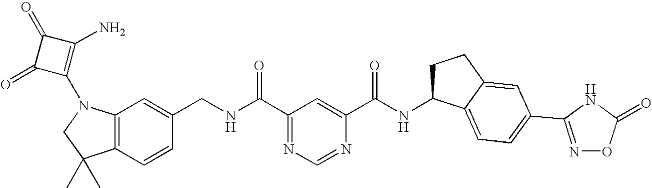 |
| 2458 | 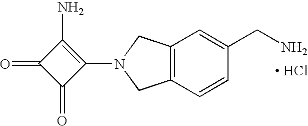 | 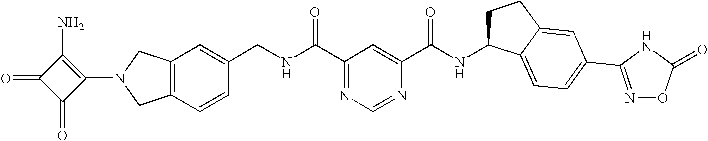 |
| 2459 | 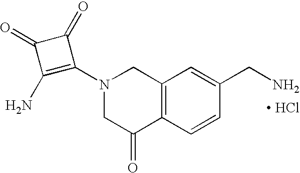 | 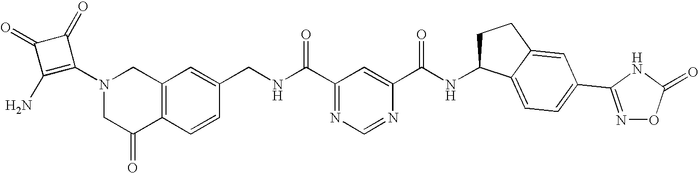 |
| 2460 | 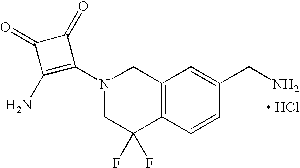 | 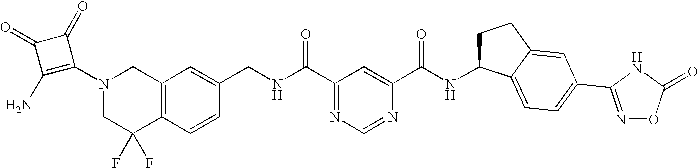 |
| 2461 | 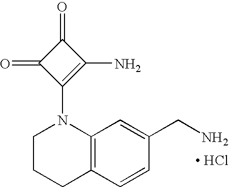 | 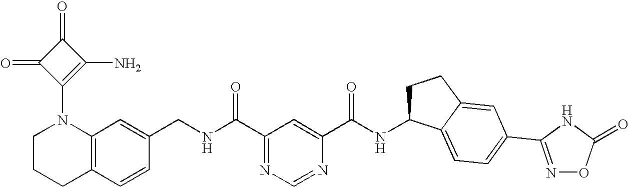 |
| 2462 | 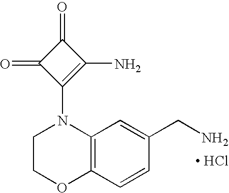 | 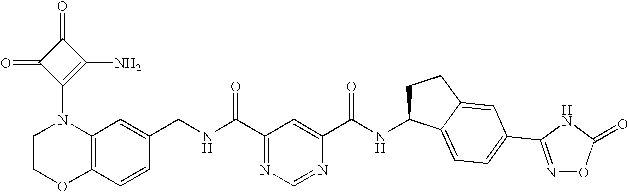 |
| 2463 | 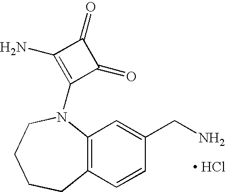 | 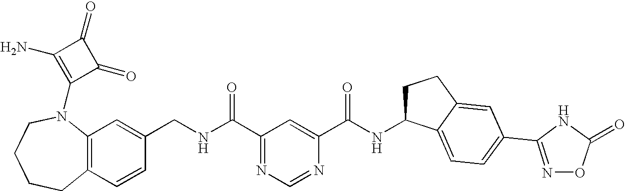 |
| 2464 | 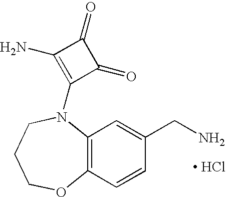 | 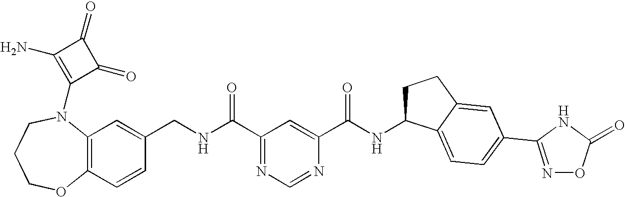 |
| 2465 | 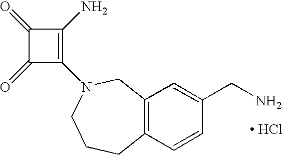 | 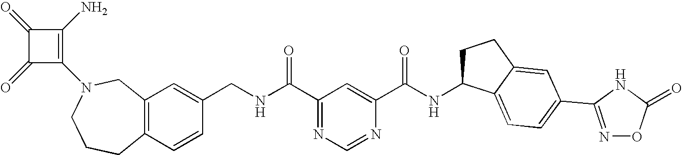 |
| 2466 | 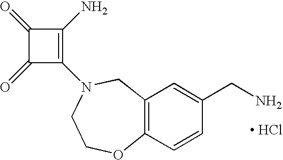 | 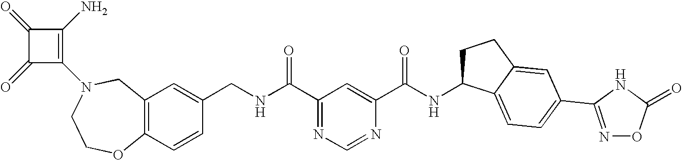 |
| 2467 | 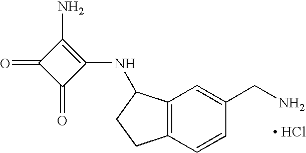 | 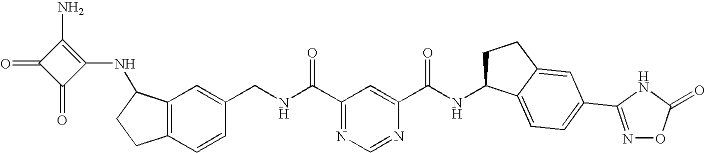 |
| 2468 | 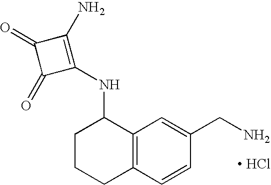 | 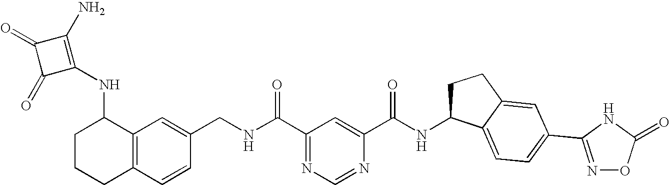 |
| 2469 | 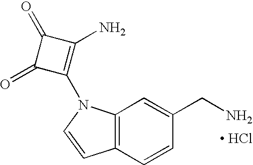 | 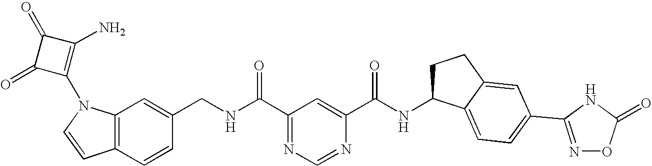 |
| 2470 | 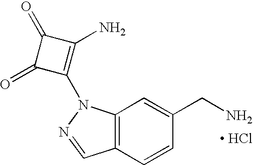 | 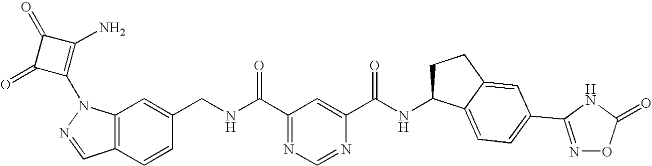 |
| 2471 | 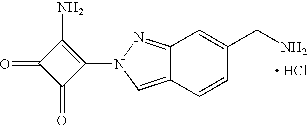 | 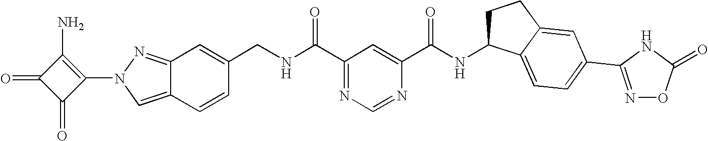 |

### Examples 2472-2474

Following a similar procedure as that described in Example 2400, except using the compounds commercially available or from the Preparative Examples indicated in Table 16 below, the following compounds were prepared.

TABLE 16

| Ex. # | Amine | Product | Yield MS |
|---|---|---|---|
| 2472 | 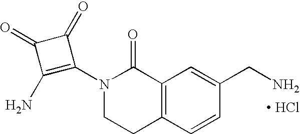 | 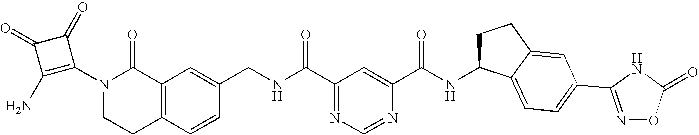 | 84% [MH]⁺ = 621 |
| 2473 | 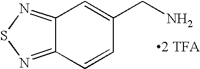 | 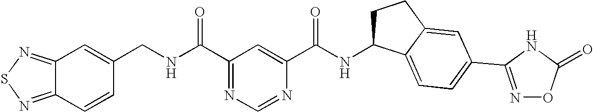 | 19% [MH]⁺ = 515 |
| 2474 | 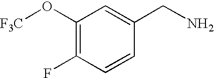 | 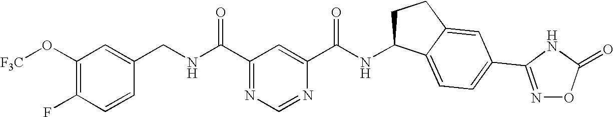 | 12% [MH]⁺ = 559 |

### Example 2500

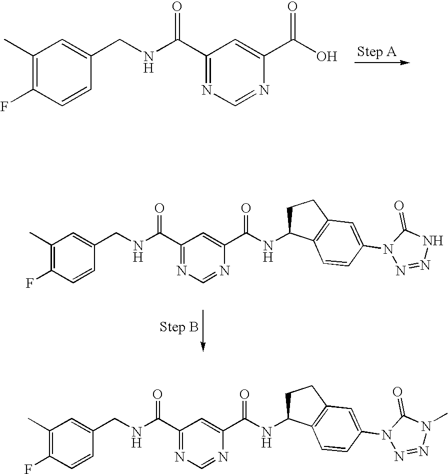 Step A

The title compound from the Preparative Example 2120, Step B (58 mg) was dissolved in THF and cooled to −10° C. N-methylmorpholine (44 μL) and isobutyl chloroformate (31 μL) were added sequentially. The reaction was kept at same temperature for 30 min. The title compound from the Preparative Example 2115, Step E (230 mg) in N,N-dimethylformamide, which was basified by N-methylmorpholine (44 μL), was added. The reaction was warmed up to room temperature in 1 h and concentrated to dryness. The crude mixture was purified by column chromatography to give the title compound (58 mg; 63%) as an off-white solid. [MNa]⁺=511.

Step B

To the intermediate from Step A above (8.5 mg) in benzene (0.75 mL) and methanol (0.25 mL) was added trimethylsilyldiazomethane (2M in diethyl ether, 9.6 [L) and stirred for 1 h. The solution was concentrated in vaccuo. The brown solid was purified by silica gel chromatography to give the title compound (8 mg; 90%) as an off-white solid. [MNa]⁺=525.

### Example 2501

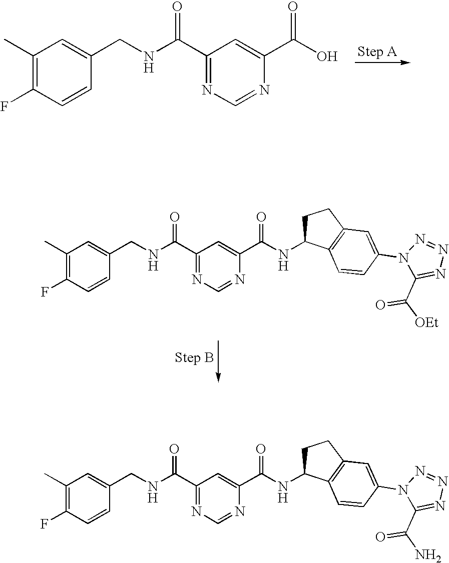 Step A

To the mixture the title compound from the Preparative Example 2120, step B (28 mg), title compound from the Preparative Example 2116, Step A (25 mg), triethylamine (138 μL) in tetrahydrofurane (2 mL) and N,N-dimethylformamide (0.2 mL) was added PyBop (51 mg). The reaction was stirred at room temperature for 2 h and diluted with ethyl acetate (10 mL). After conventional aqueous workup, the crude product was purified by column chromatography to give the intermediate (16 mg; 34%) as an off-white solid. [MH]⁺=545.

Step B

The intermediate from Step A above (5 mg) was dissolved in ammonia (7N in methanol, 3 mL) and kept overnight at room temperature. The solution was concentrated to dryness. The crude product was purified by column chromatography to give the title compound (4.5 mg; 94%). as an off-white solid. [MH]⁺=516.

### Example 2502

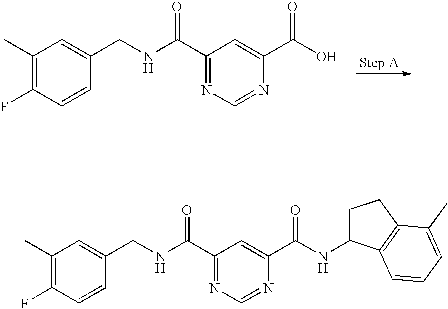 Step A

The intermediate from the Preparative Example 2120, step B (60 mg) was dissolved in THF (5 mL) and DMF (0.5 mL) and cooled to −30° C. upon which N-methylmorpholine (23° μL) was added, followed by isobutyl chloroformate (27 μL). After stirring for 1 h at −30° C., the commercially available 4-methyl-indan-1-ylamine (62 mg) was added at once. The mixture was stirred for an additional 1 h at −30° C. and then gradually warmed to room temperature upon which the mixture was concentrated under high vacuum to afford an oil. This oil was purified by flash chromatography using 20% EtOAc/CH₂Cl₂ to give the title compound (50 mg; 57%) as a colourless solid. [MH]⁺=419.

### Examples 2503-2505

Following a similar procedure as that described in Example 2502, except using the amine indicated in Table 17 below, the following compounds were prepared.

TABLE 17

| Ex. # | Amine | Product | Yield MS |
|---|---|---|---|
| 2503 | 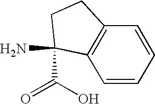 | 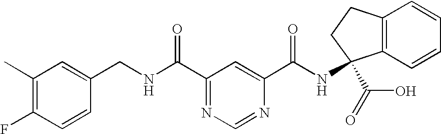 | 26% [MH]⁺ = 449 |
| 2504 | 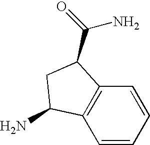 | 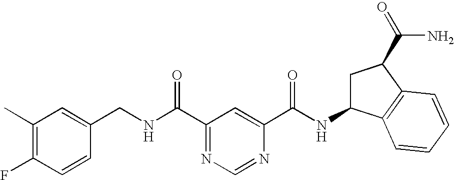 | 34% [MH]⁺ = 448 |
| 2505 | 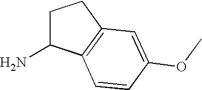 | 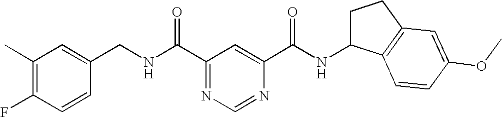 | 21% [MH]⁺ = 435 |

### Example 2506

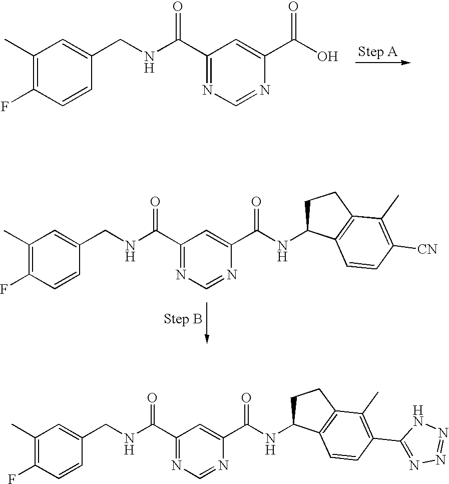 Step A

To a mixture of title compound from Preparative Example 2111 above (35 mg), the intermediate from the Preparative Example 2120, step B above (48.5 mg), bromotripyrrolidinophosphonium hexafluorophosphate (96 mg) in THF (1.7 mL) was added triethylamine (52 μL). The mixture was allowed to stir at 22° C. for 18 h. EtOAc (5 mL) and 1N aqueous hydrochloric acid (5 mL) were added. The aqueous layer was washed two times with EtOAc (5 mL). The combined organic layers were washed with a saturated aqueous solution of NaHCO₃ (5 mL), brine (5 mL), dried over MgSO₄, filtered and concentrated. The resulting residue was purified by silica gel chromatography (hexanes/ethyl acetate 1:1) to afford the intermediate (39.0 mg; 52%) as an off-white solid. [MH]⁺=444.

Step B

A mixture of the intermediate from Step A above (37.6 mg), dibutyl tinoxide (4 mg), azidotrimethylsilane (22 μL) and toluene (0.8 mL) under an atmosphere of Argon in a sealed vial was allowed to stir at 110° C. for 30 h. The reaction mixture was concentrated and purified by silica gel chromatography (CH₂Cl₂/MeOH 9:1) to give the title compound (7.0 mg; 17%) as an off-white solid. [MH]⁺=487.

### Example 2507

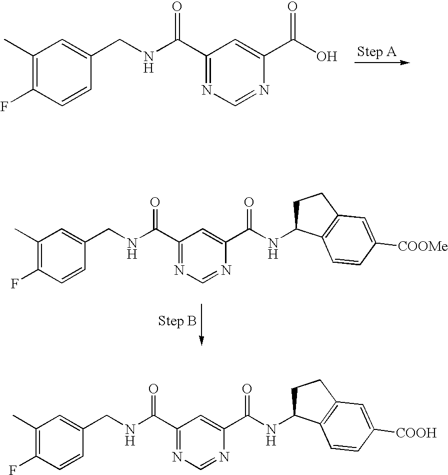 Step A

To a solution of the intermediate from the Preparative Example 2120, Step B (0.5 g) in N,N-dimethylformamide (6 mL) was added tetrahydrofurane (3 mL) and N-methylmorpholine (0.21 mL) and the mixture was chilled (−40° C.) under nitrogen. To the chilled solution was then added isobutyl chloroformate (0.25 mL) and mixture was stirred at between −40° C. to −20° C. for 2 h. To the chilled solution was added the title compound from the Preparative Example 2105, step B (0.43 g) dissolved in tetrahydrofurane (3 mL) and the mixture was allowed to stir at −40° C. to −20° C. for 2 h and then slowly warmed to room temperature. To the mixture was then added water (2-3 drops) and stirred for 1 h. The mixture was concentrated and resulting solid purified by column chromatography (silica, 10% hexane/dichloromethane, then 10% diethyl ether/dichloromethane) to give the intermediate (0.5 g; 63%). [MH]⁺=463.

Step B

To the intermediate of Step A above (0.4 g), dissolved in tetrahydrofurane (3 mL) was added 1N KOH (3 mL) and the mixture was stirred at room temperature for 15 h. The mixture was concentrated and the resulting solid was triturated with 10% dichloromethane/diethyl ether and then washed with 1N hydrochloric acid. The resulting solid was filtered to give the title compound (0.33 g; 86%). ¹H-NMR (DMSO) δ=2.2 (s, 3 H), 2.9-3.2 (m, 4 H), 4.5 (d, 2 H), 5.70 (q, 1 H), 7.0-7.4 (m, 4 H), 7.80 (d, 1 H), 7.85 (s, 1 H), 8.50 (s, 1 H), 9.40 (m, 2 H), 9.65 (t, 1 H).

### Examples 2508-2509

Following a similar procedure as that described in Example 2507, except using the amine indicated in Table 18 below, the following compounds were prepared.

TABLE 18

| Ex. # | Amine | Product | Yield MS |
|---|---|---|---|
| 2508 | 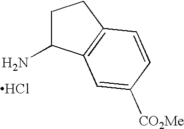 | 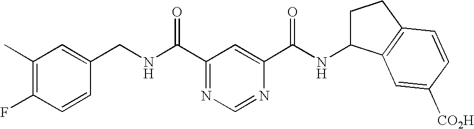 | 17% [MH]⁺ = 449 |
| 2509 | 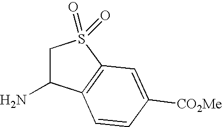 | 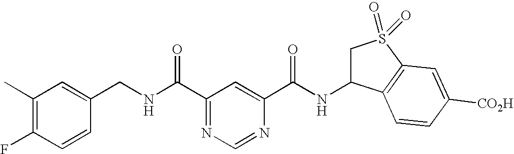 | 58% [MH]⁺ = 499 |

### Example 2510

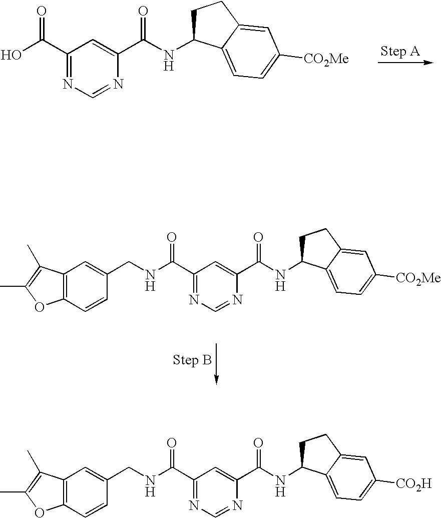 Step A

A solution of the title compound from the Preparative Example 2118, Step B (34 mg), the title compound from the Preparative Example 2042, Step D, 1-ethyl-3-(3-dimethylaminopropyl)carbodiimide (25 mg), 1-hydroxy-benzotriazole (18 mg) and N-methylmorpholine (50 μL) in DMF (4 mL) was stirred at room temperature overnight. Then the mixture was concentrated to dryness and the residue was dissolved in ethyl acetate and washed with saturated NaHCO₃, aqueous IN hydrochloric acid and brine. The organic phase was separated, dried over MgSO₄, filtered and concentrated. The residue was purified by column chromatography (silica, cyclohexane/ethyl acetate 6:4) to afford the title compound as colourless solid (61 mg; quantitative). [MH]⁺=499.

Step B

To the intermediate of Step A above (61 mg), dissolved in tetrahydrofurane (2 mL) was added a 0.5N lithium hydroxide solution (1 mL) and the mixture was stirred at room termperature overnight. The mixture was concentrated and acidified with 1N hydrochloric acid (0.5 mL). The resulting solid was filtered to give the title compound (40.7 mg; 84%). [MH]⁺485.

### Examples 2511-2519

Following a similar procedure as that described in Example 2510, except using the compounds from the Preparative Examples indicated in Table 19 below, the following compounds were prepared.

TABLE 19

| Ex. # | Amine | Product | Yield MS |
|---|---|---|---|
| 2511 | 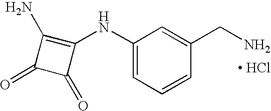 | 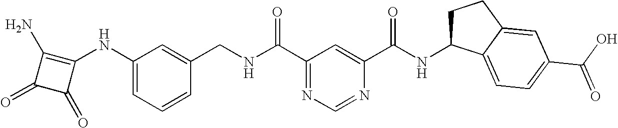 | 5% [MH]⁺ = 527 |
| 2512 | 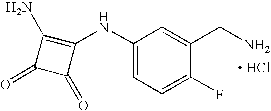 | 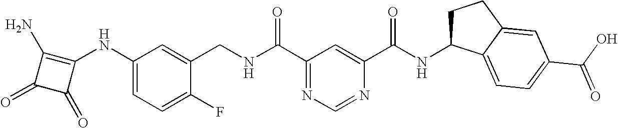 | 38% [MH]⁺ = 545 |
| 2513 | 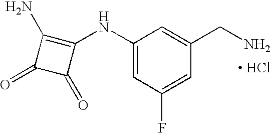 | 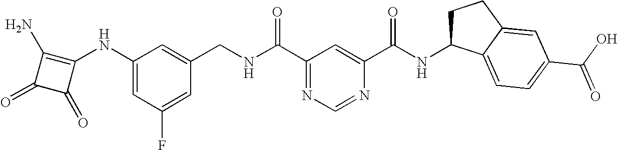 | 48% [MH]⁺ = 545 |
| 2514 | 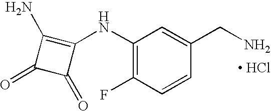 | 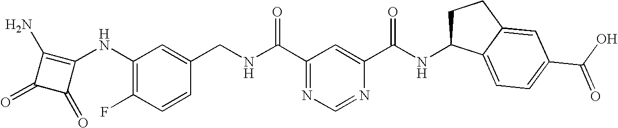 | 20% [MH]⁺ = 545 |
| 2515 | 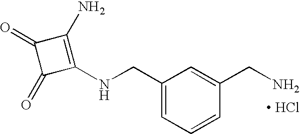 | 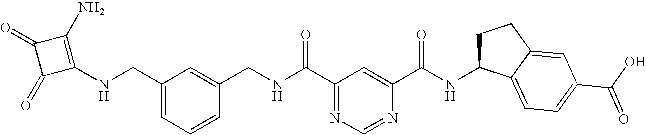 | 7% [MH]⁺ = 541 |
| 2516 | 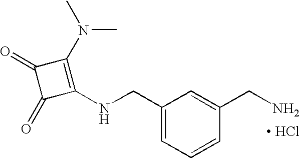 | 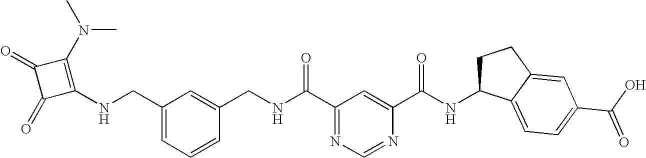 | 41% [MH]⁺ = 569 |
| 2517 | 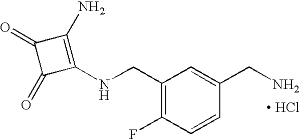 | 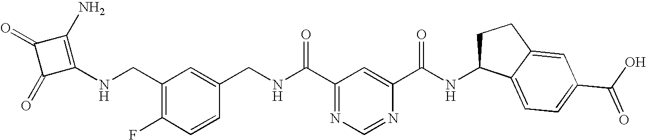 | 4% [MH]⁺ = 559 |
| 2518 | 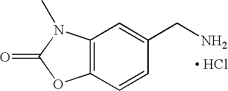 | 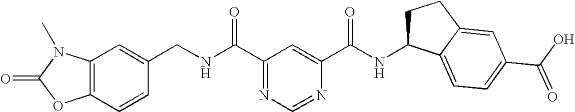 | 32% [MH]⁺ = 488 |
| 2519 | 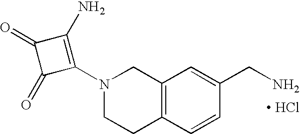 | 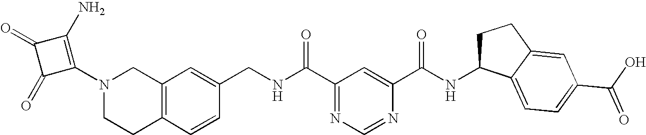 | 4% [MH]⁺ = 567 |

### Example 2520

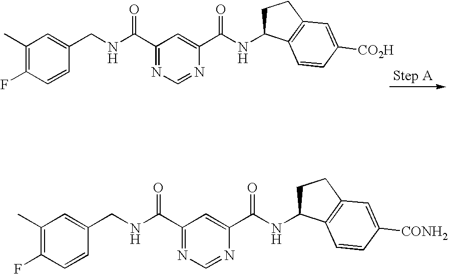 Step A

The title compound of Preparative Example 2507, Step B (80 mg) was dissolved in dry dichloromethane (5 mL) and N,N-dimethylformamide (0.1 mL) and was chilled at −30° C. To the chilled solution was added oxalyl chloride (18 μL) and mixture was stirred at between −30° C. to −10° C. for 1.5 h and then at room temperature for 30 min. The mixture was then concentrated and the resulting oil was dissolved in tetrahydrofurane (2 mL) and the solution was added to condensed ammonia and the mixture was allowed to stir at between −30° C. to −20° C. for 10 min and then warmed to room temperature over 2 h. The mixture was evaporated and the resulting solid purified by preparative thin layer chromatography (silica, 10% methanol/dichloromethane) to give the title compound (40 mg; 52%). [MH]⁺=448.

### Example 2521

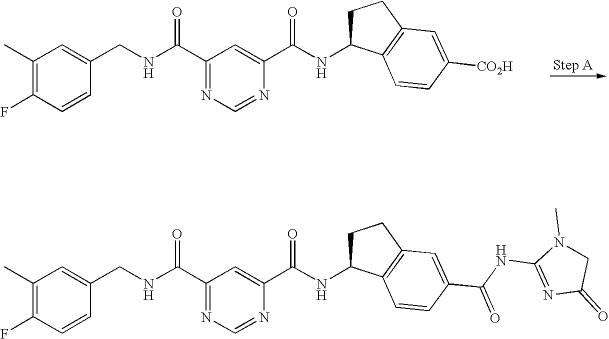 Step A

The title compound of Preparative Example 2507, Step B (0.15 g) was dissolved in dry dichloromethane (5 mL) and N,N-dimethylformamide (0.2 mL) and was chilled at −30° C. To the chilled solution was added oxalyl chloride (32 μL) and the mixture was stirred at between −30° C. to −10° C. for 1.5 h and then at room temperature for 30 min. The mixture was then concentrated and the resulting oil was dissolved in tetrahydrofurane (0.5 mL) and the solution was added to commercially available 2-amino-1-methyl-1,5-dihydro-imidazol-4-one hydrochloride (32 mg) dissolved in N-methylmorpholine (75 μL) and N,N-dimethylformamide (0.5 mL) and the mixture was allowed to stir at between −30° C. to −20° C. for 10 min and then warmed to room temperature over 2 h. The mixture was evaporated and the resulting solid purified by preparative thin layer chromatography (silica, 10% methanol/dichloromethane) to give the title compound (34 mg; 41%). [MH]⁺=544.

### Example 2522

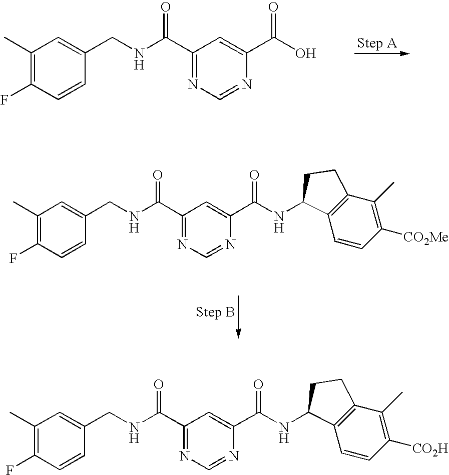 Step A

To a solution of the intermediate of Preparative Example 2120, Step B (28.7 mg) and N,N-dimethylformamide (2 μL) in CH₂Cl₂ (1 mL) at 0C was added oxalyl chloride (17 μL). The solution was allowed to warm to 22° C. and stirred for 2 h. The solution was concentrated and the resulting residue was dissolved in CH₂Cl₂ (1 mL). The resulting solution was cannulated into a mixture of the intermediate of Preparative Example 2110, Step K (20.0 mg) and triethylamine (56 μL) in CH₂Cl₂ (1 mL) and the mixture was stirred for 2 h at which time it was a homogeneous solution. Silica gel (500 mg) was added and the mixture was concentrated and purified by silica gel chromatography (hexanes/ethyl acetate 1:1) to afford the intermediate (29.0 mg; 61%) as an off-white solid. [MH]⁺=477.

Step B

A solution of the intermediate from step A above (29.0 mg) in THF (240 μL), MeOH (120 μL), and IN aqueous solution of LiOH (120 μL) was stirred at 50° C. for 1 h. The solution was concentrated to remove all MeOH and the resulting residue was dissolved in THF (200 μL) and acidified with concentrated hydrochloric acid (20 μL). The mixture was concentrated and purified by silica gel chromatography (CH₂Cl₂/MeOH 9:1) to afford the title compound (15.0 mg; 53%) as an off-white solid. [MH]⁺=463.

### Examples 2523-2538

If one were to follow a similar procedure as that described in Example 2510, Step A and Step B, except using the amine from the Preparative Examples indicated in Table 20 below, the following title compounds would be obtained.

TABLE 20

| Ex. # | Amine | Product |
|---|---|---|
| 2523 | 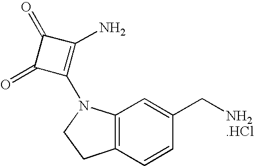 | 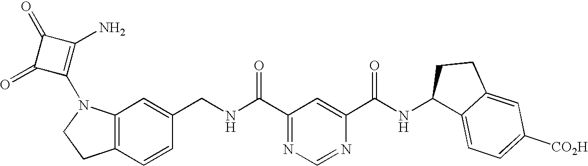 |
| 2524 | 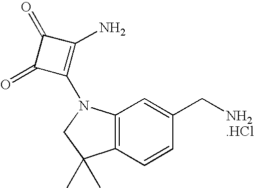 | 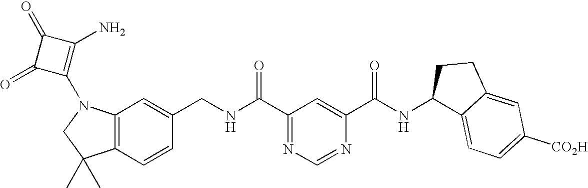 |
| 2525 | 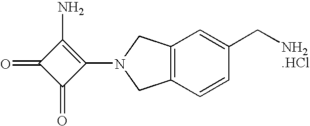 | 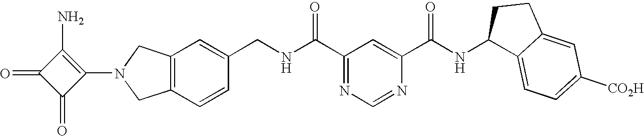 |
| 2526 | 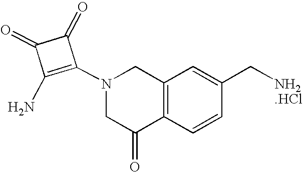 | 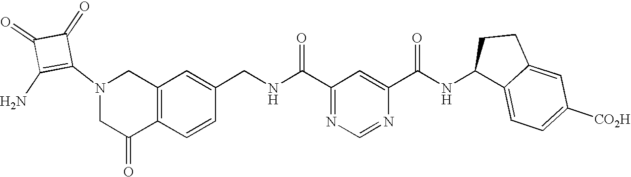 |
| 2527 | 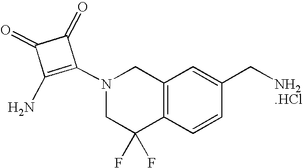 | 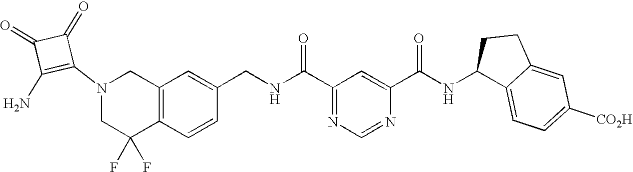 |
| 2528 | 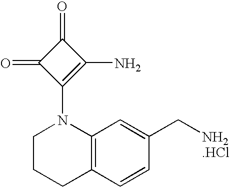 | 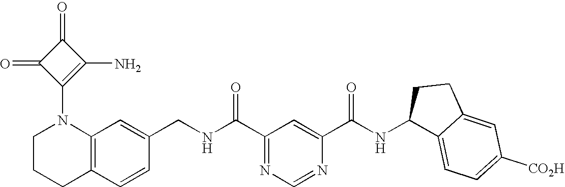 |
| 2529 | 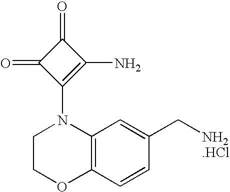 | 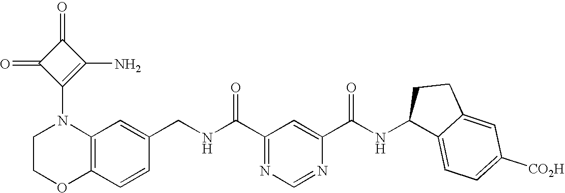 |
| 2530 | 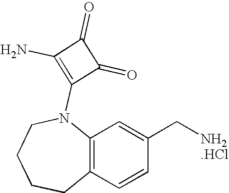 | 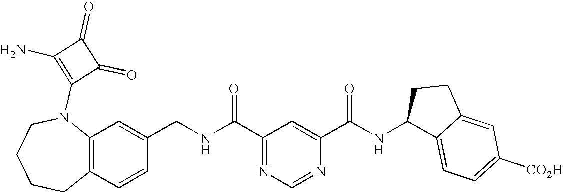 |
| 2531 | 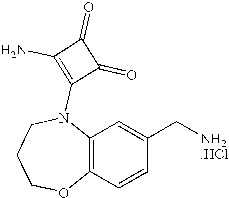 | 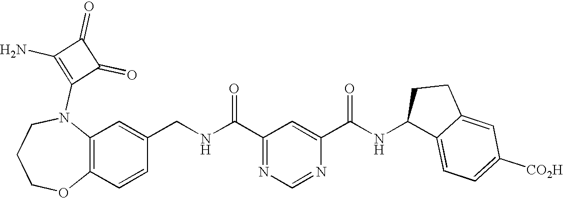 |
| 2532 | 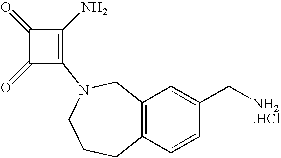 | 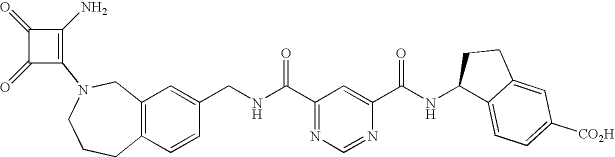 |
| 2533 | 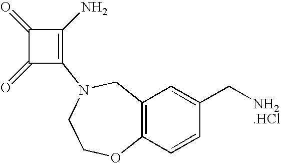 | 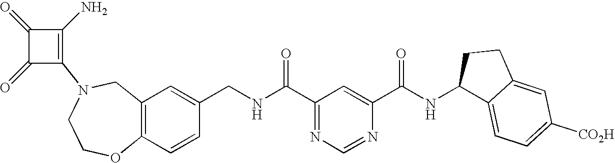 |
| 2534 | 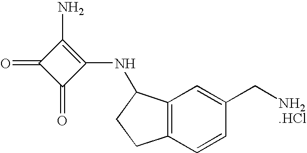 | 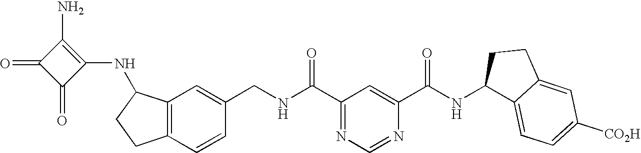 |
| 2535 | 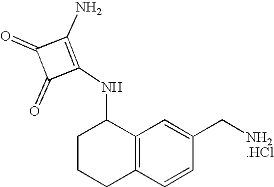 | 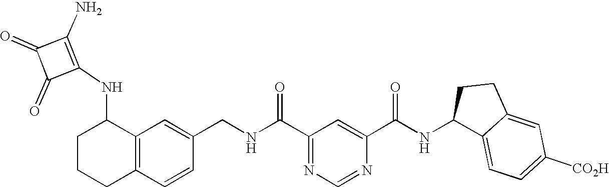 |
| 2536 | 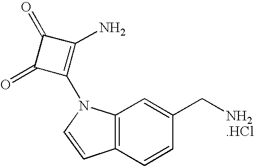 | 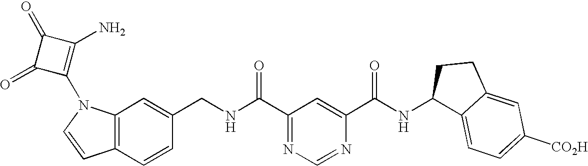 |
| 2537 | 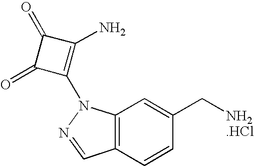 | 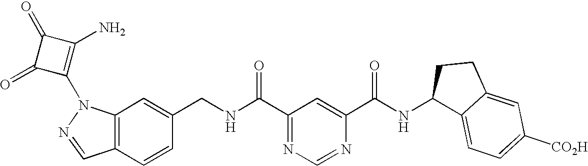 |
| 2538 | 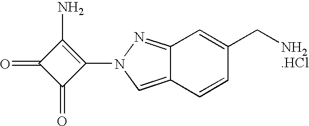 | 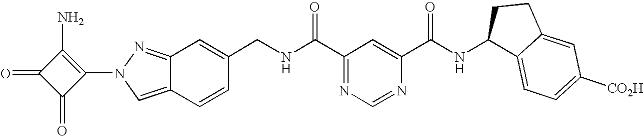 |

### Examples 2539-2555

If one were to follow a similar procedure as that described in Example 2510, Step A and Step B, except using the intermediate from the Preparative Example 2122 and the amine from the Preparative Examples indicated in Table 21 below, the following title compounds would be obtained.

TABLE 21

| Ex. # | Amine | Product |
|---|---|---|
| 2539 | 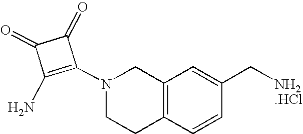 | 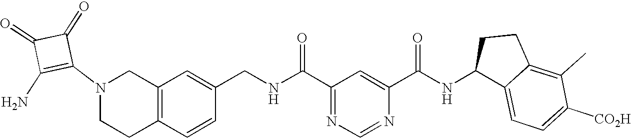 |
| 2540 | 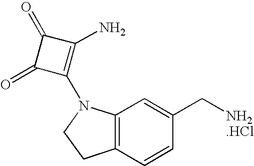 | 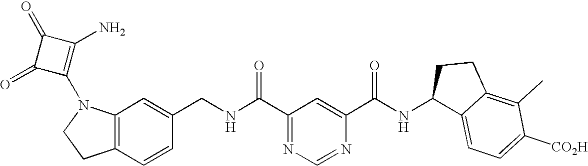 |
| 2541 | 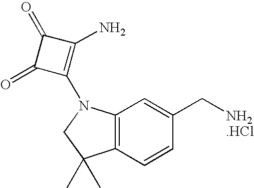 | 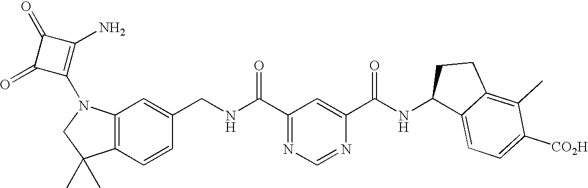 |
| 2542 | 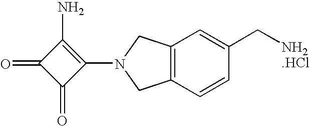 | 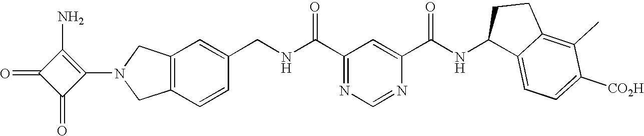 |
| 2543 | 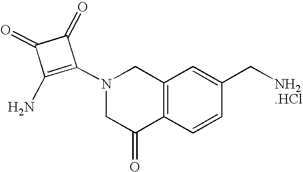 | 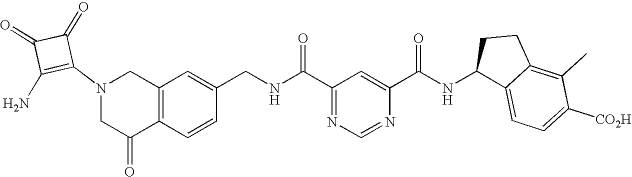 |
| 2544 | 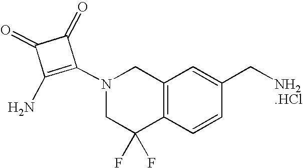 | 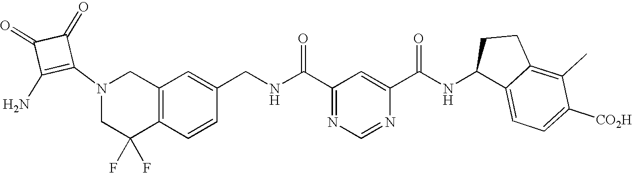 |
| 2545 | 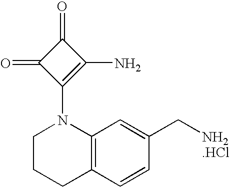 | 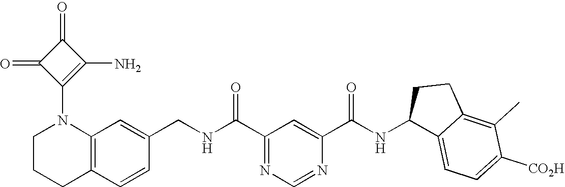 |
| 2546 | 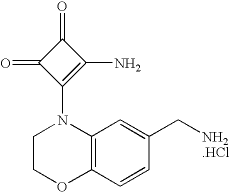 | 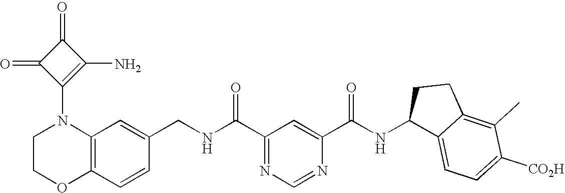 |
| 2547 | 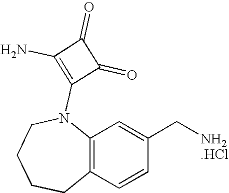 | 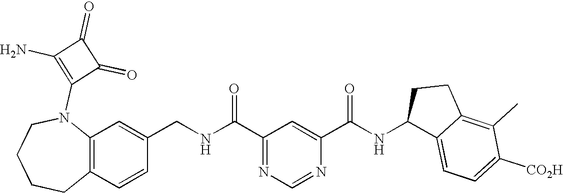 |
| 2548 | 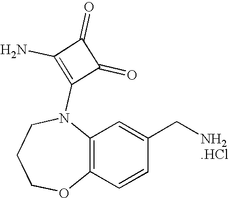 | 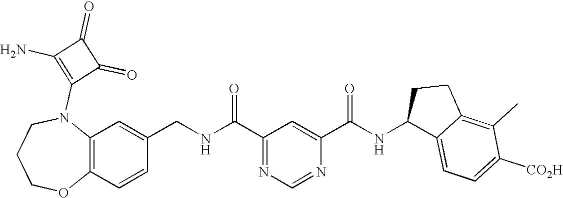 |
| 2549 | 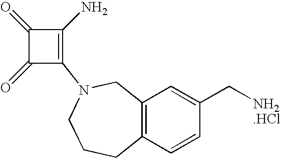 | 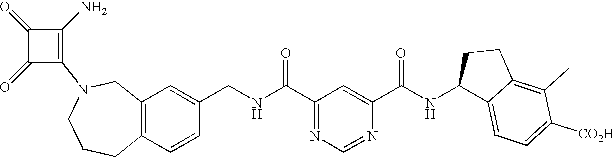 |
| 2550 | 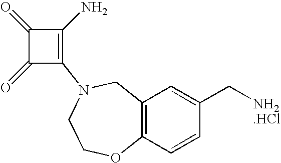 | 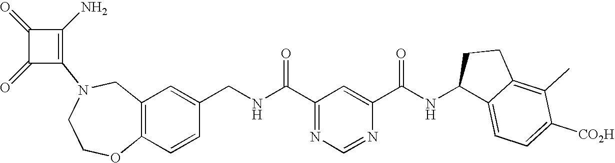 |
| 2551 | 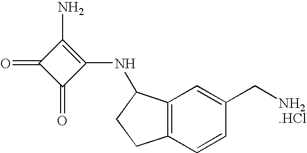 | 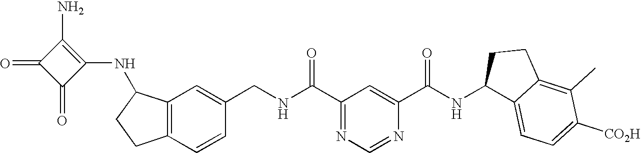 |
| 2552 | 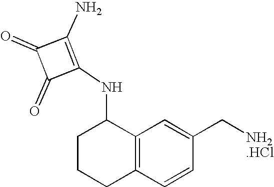 | 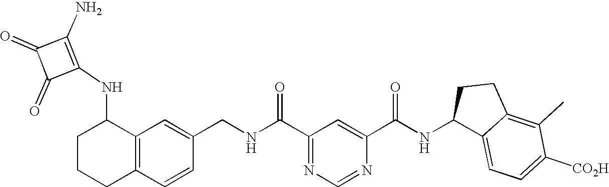 |
| 2553 | 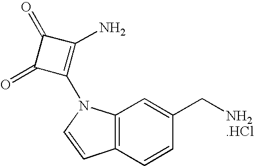 | 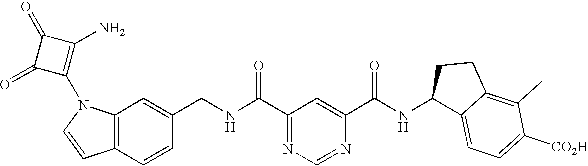 |
| 2554 | 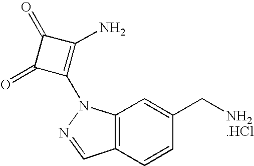 | 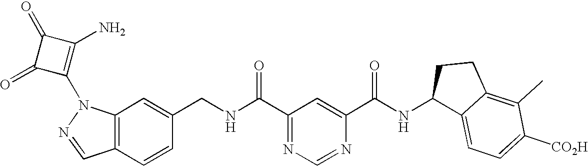 |
| 2555 | 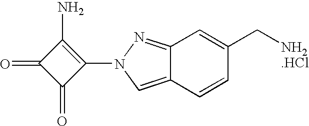 | 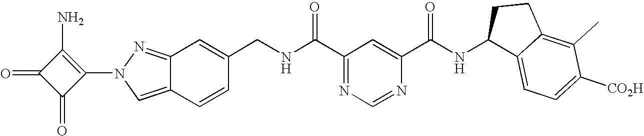 |

### Example 2556

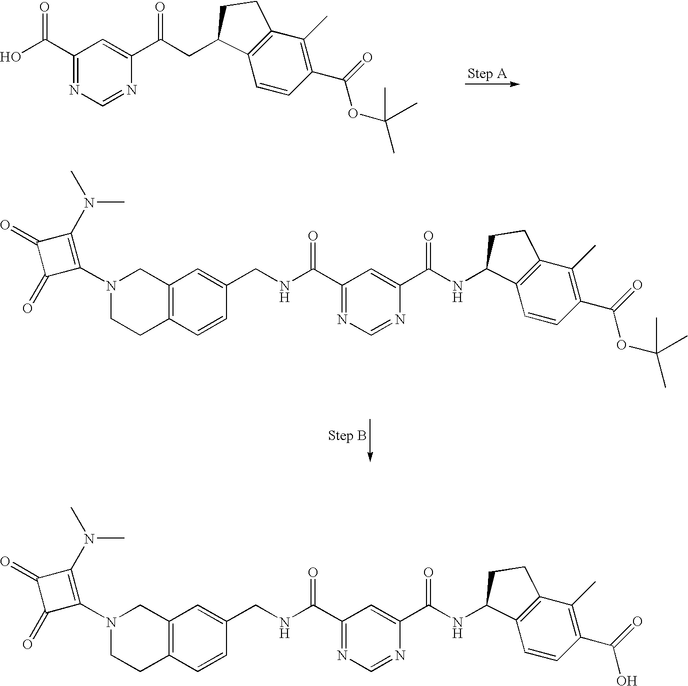 Step A

The title compound from Preparative Example 2125, Step E above (120 mg) was dissolved in dry dimethylformamide (3 mL). After adding O-(7-azabenzotriazol-1-yl)-N,N,N′,N′-tetramethyluronium hexafluorophosphate (137 mg), 1-Hydroxy-7-azabenzotriazole (50 mg) and the title compound from the Preparative Example 2061 (117 mg) together with diisopropyl ethylamine (150 μL), the mixture was stirred at room temperature (5 h). The solvent was removed, the residue was dissolved in ethyl acetate and washed with a 0.01 M solution of hydrochloric acid. The organic layer was dried (MgSO₄) and concentrated to afford the title compound as a colourless solid (95 mg; 57%.). [MH]⁺=665.

Step B

To a solution of the title compound from Step A above (90 mg) in dry dichloromethane (4 mL) was added trifluoroacetic acid (1 mL). The mixture was stirred at room temperature for 2 h. The solvent was removed and the residue was concentrated and purified by column chromatography (silica, chloroform/methanol 9:1) to afford the title compound (28 mg; 33%) as a colourless solid. [MH]⁺=609.

### Examples 2557-2562

Following a similar procedure as that described in Example 2556, Step A and Step B, except using the amine from the Preparative Examples indicated in Table 22 below, the following title compounds were prepared.

TABLE 22

| | | | Yield |
|---|---|---|---|
| Ex. # | Amine | Product | MS |
| 2557 | 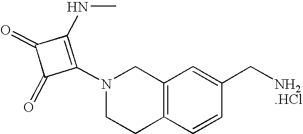 | 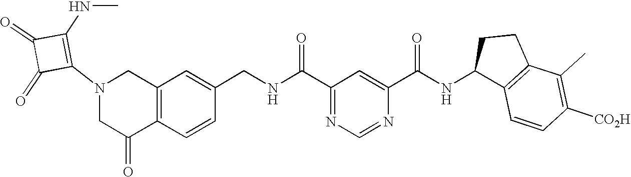 | 35% [MH]⁺ = 595 |
| 2558 | 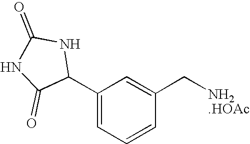 | 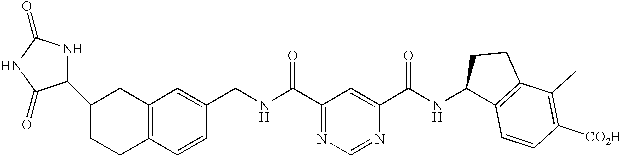 | 76% [MH]⁺ = 529 |
| 2559 | 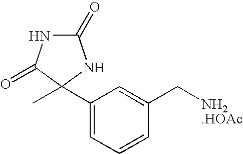 | 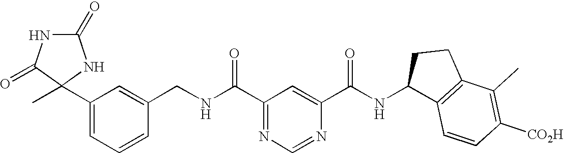 | 4% [MH]⁺ = 543 |
| 2560 | 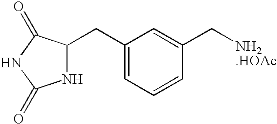 | 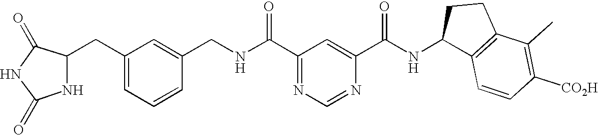 | 53% [MH]⁺ = 543 |
| 2561 | 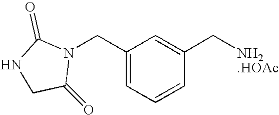 | 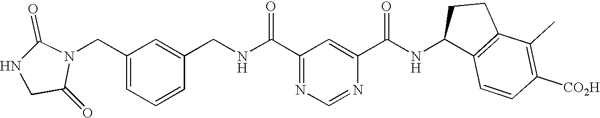 | 43% [MH]⁺ = 543 |
| 2562 | 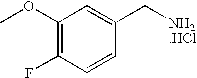 | 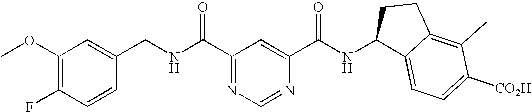 | 70% [MH]⁺ = 479 |

### Example 2563

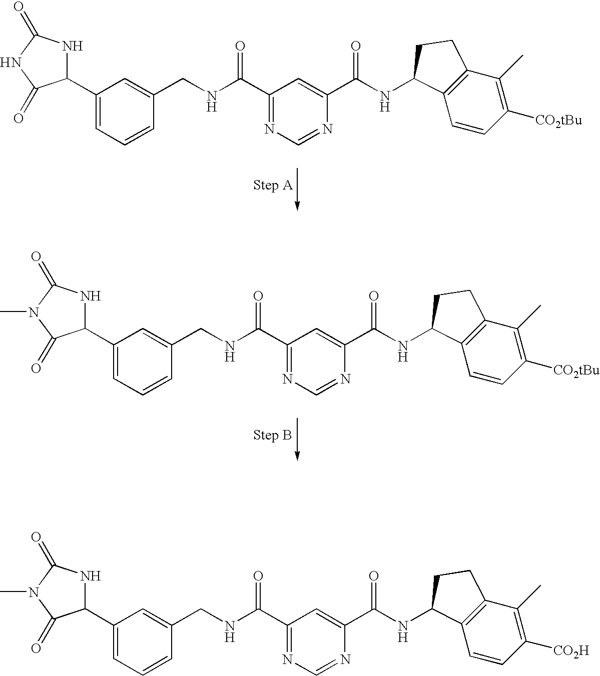 Step A

To the intermediate from the Preparative Example 2558 (8 mg) was added trimethylsilyl azidomethane (8.2 μL, 2M in diethyl ether) at room temperature in benzene and methanol (0.3 mL, 3:1). After 1 h, another portion of trimethylsilyl azidomethane (8.2 μL, 2M in diethyl ether) was added. The reaction was stirred for another 2 h until reaction went to completion. The solution was concentrated and the product was used without further purification.

Step B

The title compound from Step A above was treated similar as described in the Preparative Example 2556, Step B to afford the title compound as a colourless solid. [MH]⁺=543.

### Example 2600

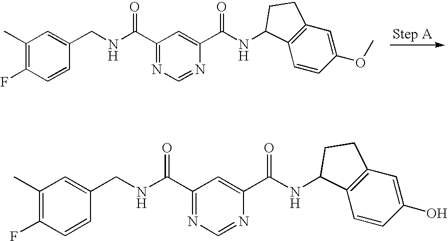 Step A

To a solution of the title compound of Example 2505 (23.1 mg) in CH₂Cl₂ (0.5 mL) at 0° C. was added BBr₃ (30.2 mL). The solution was allowed to warm to 22° C. and stirred for 1.5 h. 1N hydrochloric acid (5 mL) was added and the aqueous layer was washed with CH₂Cl₂ (3×5 mL). The combined organic layers were dried over anhydrous MgSO₄, filtered and concentrated and purified by silica gel chromatography (hexanes/EtOAc 1:1) to yield the title compound (17.8 mg; 80%) as a colourless solid. [MH]⁺=421.

### Example 2601

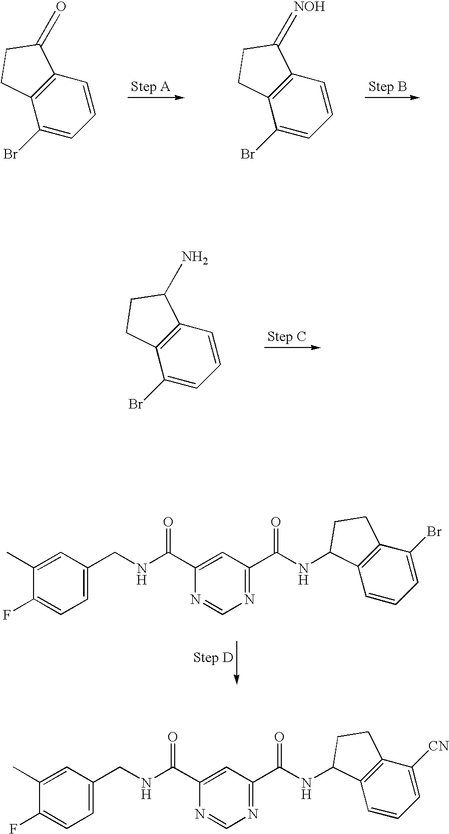 Step A

The intermediate of Preparative Example 2120, Step B (102 mg), 4-bromo-2,3-dihydro-1H-inden-1-amine (75 mg), 1-ethyl-3-(3-dimethylaminopropyl)-carbodiimide (102 mg), 1-hydroxybenzotriazole (48 mg) and potassium carbonate (224 mg) were dissolved in THF (5 mL) and stirred for 15 h. The mixture was diluted with ethyl acetate, washed with saturated sodium bicarbonate, ammonium chloride and brine, dried (MgSO₄), concentrated and purified by column chromatography (silica, hexanes/EtOAc) to afford the title compound (111 mg) as a solid. [MH]⁺=483.

Step B

The title compound from Step A above (93 mg), bis(dibenzylideneacetone)palladium (8.8 mg) and 1,1′-bis(diphenylphosphino)propane (21 mg) were dissolved in DMF (5 mL) and heated to 80° C. Zinc(II) cyanide (27 mg) in DMF (1.5 mL) was added to the reaction mixture dropwise. The mixture was stirred for 15 h, concentrated and purified by column chromatography (silica, hexanes/EtOAc) to afford the title compound (60 mg) as colourless solid. [MR]⁺=430.

### Example 2602

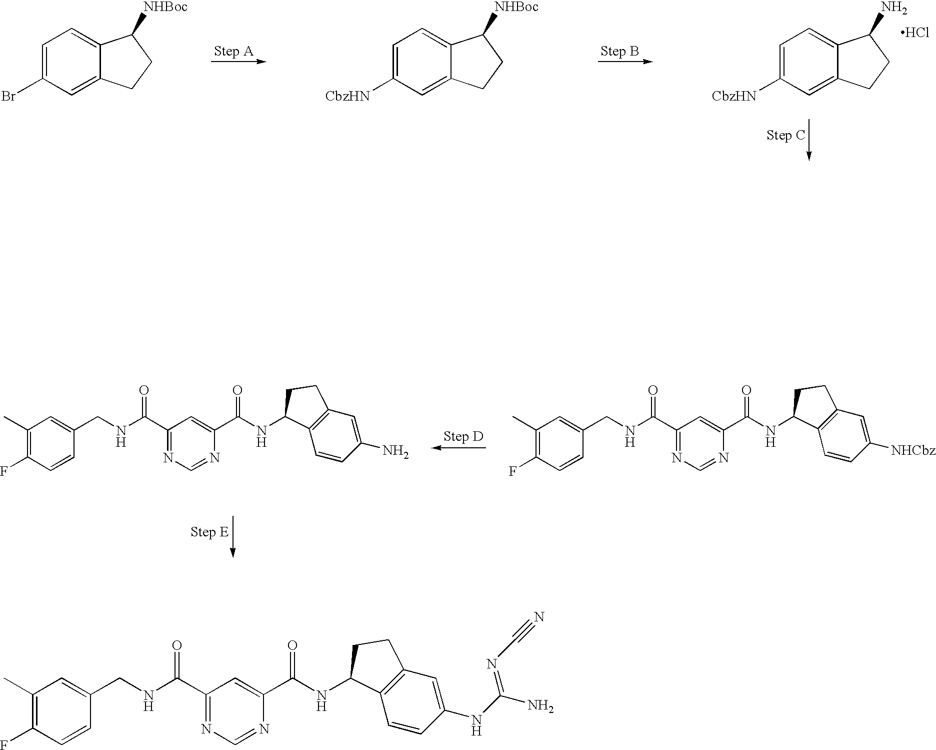 Step A

(5-Bromo-indan-1-yl)-carbamic acid tert-butyl ester (1.55 g), benzylcarbamate (904 mg), bis(dibenzylideneacetone)palladium (114 mg), Xantphos (217 mg) and caesium carbonate (2.281 g) were weighed into a small flask. Anhydrous dioxane (25 mL) was added under an argon atmosphere and the reaction was heated at 95° C. for 18 hours. Volatiles were removed under reduced pressure and the residue taken up in ethyl acetate and dry packed on silica. Purification by column chromatography (25% ethyl acetate in hexane) resulted in isolation of the product as a colourless solid (930 mg). [MH]⁺=383.

Step B

The intermediate from Step A above (930 mg) was dissolved in 4N HCl in dioxane (10 mL) for 16 h. Volatiles were removed under reduced pressure and the residue washed with diethyl ether and dried under vacuum to give the intermediate (445 mg) as a grey solid. [M-Cl]⁺=283.

Step C

The intermediate from the Preparative Example 2120, Step B (400 mg) was dissolved in DMF (7 mL) and THF (5 mL). N-methylmorpholine (175 mg) was added and the solution was cooled to −40° C. Isobutyl chloroformate (207 mg) was added and the reaction was stirred for 90 min at −30° C. to −40° C. The intermediate from step B above (440 mg) and N-methylmorpholine (200 mg) were slurried in THF (7 mL) and transferred by pipette to the mixed anhydride. The reaction was allowed to warm to room temperature over 18 h. Volatiles were removed under reduced pressure and the residue partitioned between ethyl acetate and water. The organic layer was concentrated and the residue purified by column chromatography (5% methanol in dichloromethane) to yield the intermediate (200 mg) as an off-white solid. [MH]⁺=554.

Step D

The intermediate from Step C above was slurried in acetic acid (2 mL). Hydrogen bromide (33% solution in acetic acid, 0.5 mL) was added. After 1.5 h, a further amount of hydrogen bromide (33% solution in acetic acid) was added and the reaction was stirred for 1 h. Volatiles were removed under reduced pressure. The residue was washed with diethyl ether (40 mL) and then partitioned between aqueous sodium bicarbonate and dichloromethane. The organic layer was concentrated and the residue was purified to give the intermediate (125 mg) as a yellow oil. [MH]⁺=421.

Step E

The intermediate from Step D above was dissolved in ethanol (2 mL) and dimethyl N-cyanodithioiminocarbonate (150 mg) was added. The reaction was heated at 80° C. overnight. The resulting precipitate was filtered and the solid was washed with a small amount of ethanol and diethyl ether. The crude product was purified by column chromatography (5% methanol in dichloromethane) to give an off-white solid (50 mg). The resulting solid was heated in ammonia (7N in methanol, 10 mL) to 50° C. for 36 h. The reaction was dry packed on silica and purified by column chromatography (5% methanol in dichloromethane) to give the title compound (33 mg) as an off-white solid. [MH]⁺=487.

### Example 2603

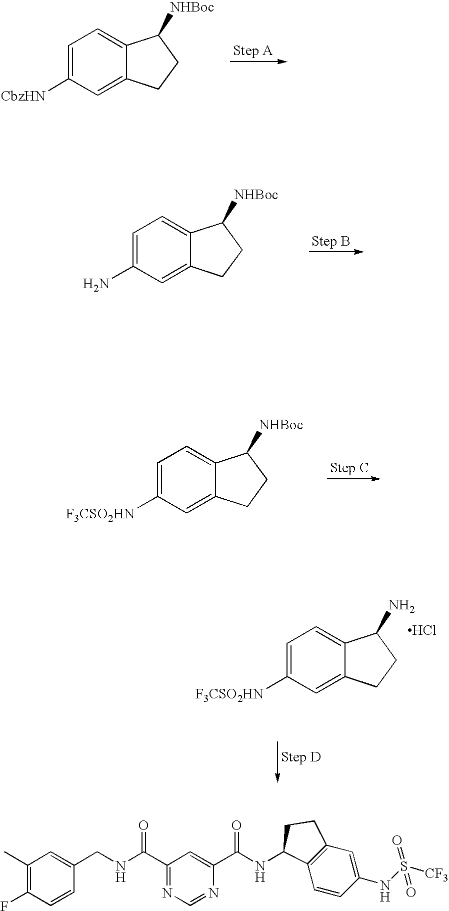 Step A

The intermediate from Example 2602, Step A (650 mg) was dissolved in ethanol (40 mL) and palladium on charcoal (10 wt %, 250 mg) was added. The reaction was placed on a Parr shaker-type hydrogenation apparatus and pressurized with 60 psi hydrogen. After 36 h, the reaction was filtered and dry packed on silica. Purification by flash chromatography (25% ethyl acetate in hexane) gave the intermediate (300 mg) as a colourless oil. [MH]⁺=249.

Step B

The intermediate from Step A above (150 mg) was dissolved in dichloromethane (3 mL) and triethylamine (122 mg) was added. The solution was cooled to −78° C. and trifluoromethanesulfonic acid anhydride (164 mg) was added. The reaction was allowed to warm to room temperature over 30 min, then diluted with dichloromethane and 0.1N hydrochloric acid. The organic layer was concentrated and the residue purified by column chromatography to give the intermediate (205 mg) as a colorless oil. [MNa]⁺=403.

Step C

The intermediate from Step B above (205 mg) was dissolved in hydrogen chloride (4N in dioxane, 2 mL). The reaction was stirred for 2 h, volatiles were removed under reduced pressure and the residue washed with diethyl ether to give the intermediate (135 mg) as a solid whose NMR was consistent with the presence of one-half equivalents of dioxane. ¹H-NMR (DMSO) δ=8.4 (br, 3 H), 7.7 (d, 1 H), 7.25 (m, 2 H), 4.75 (m, 1 H), 3.2 (m, 1 H), 2.95 (m, 1 H), 2.40 (m, 1 H), 2.05 (m, 1 H).

Step D

The intermediate from Preparative Example 2120, Step B (114 mg) was dissolved in DMF (0.5 mL) and THF (2 mL). N-methylmorpholine (81 mg) was added and the solution was cooled to −40° C. Isobutyl chloroformate (55 mg) was added and the reaction was stirred for 90 min at −30° C. to −40° C. The intermediate from step C above (125 mg) and N-methylmorpholine (161 mg) were slurried in THF (2 mL) and transferred by pipette to the mixed anhydride. The reaction was allowed to warm to room temperature over 18 h. Volatiles were removed under reduced pressure and the residue was partitioned between ethyl acetate and aqueous ammonium chloride. The organic layer was concentrated and the residue purified by column chromatography (5% methanol in dichloromethane) to yield the title compound (135 mg) as an off-white solid. [MH]⁺=552.

### Example 2604

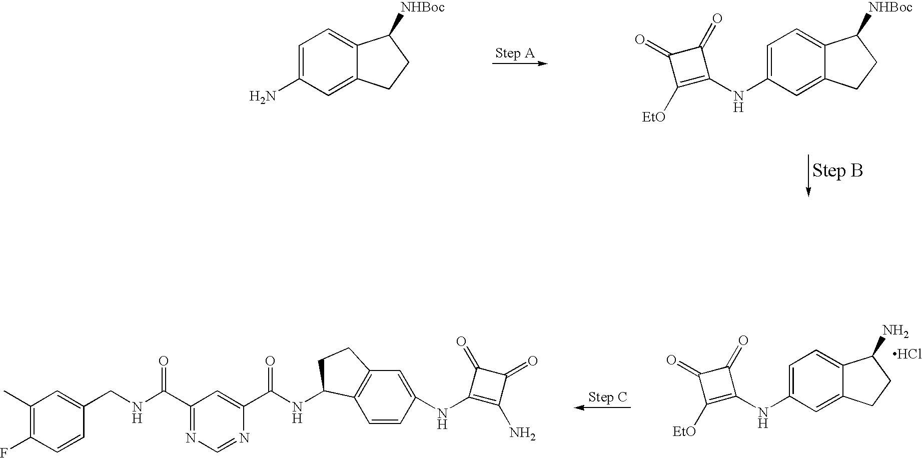 Step A

The intermediate from Example 2603, Step A (550 mg) was dissolved in ethanol (2 mL). 3,4-Diethoxy-3-cyclobutene-1,2-dione (0.70 g) was added and the reaction was heated at 65° C. overnight. Volatiles were removed under reduced pressure and the residue was washed with diethyl ether/hexanes (1:1, 10 mL) and dried under vacuum to yield the intermediate (605 mg) as a solid. [MNa]⁺=395.

Step B

The intermediate from Step A above (100 mg) was dissolved in hydrogen chloride (4N in dioxane, 5 mL). After 2 h, volatiles were removed under reduced pressure. The residue was washed with diethyl ether and dried under vacuum, yielding the intermediate (80 mg) as a grey solid. [M-NH₃Cl]⁺=256, [M-Cl]⁺=273.

Step C

The intermediate from Preparative Example 2120, Step B (15 mg), 1-(3-dimethylaminopropyl)-3-carbodiimide hydrochloride (11 mg) and 1-hydroxy-benzotriazole (8 mg) were weighed into a flask. DMF (0.5 mL) and THF (0.5 mL) were added and the mixture was stirred for 1 h. The intermediate from Step B above (10 mg) was added along with triethylamine. The reaction was stirred overnight, diluted with ethyl acetate and washed with water and diluted hydrochloric acid. The residue was purified by column chromatography (10% methanol in dichloromethane) to give the solid intermediate (14 mg, [MH]⁺=544). This purified squarate ester was dissolved in THF (1 mL) and ammonia (7N in methanol, 200 μL) was added. The reaction was allowed to stir for 36 h and the resulting precipitate isolated by centrifugation of the reaction mixture followed by decanting the supernatant to afford the title compound (8 mg) as a solid. [MH]⁺=515.

### Example 2605

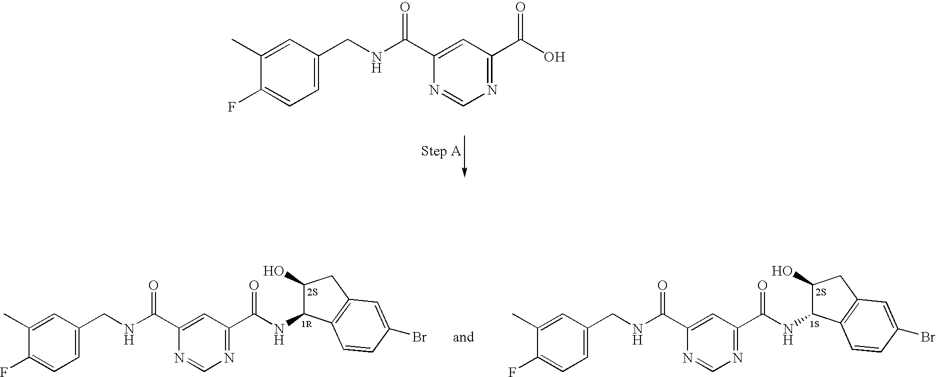 Step A

The intermediate from Preparative Example 2120, Step B (196 mg), (2S)-1-amino-5-bromo-2,3-dihydro-1H-inden-2-ol (154 mg), 1-ethyl-3-(3-dimethylaminopropyl)-carbodiimide (195 mg), 1-hydroxybenzotriazole (91 mg) and potassium carbonate (214 mg) were dissolved in THF (5 mL) and stirred for 15 h. The mixture was diluted with ethyl acetate, washed with saturated sodium bicarbonate, ammonium chloride and brine, dried (MgSO₄), concentrated and purified by column chromatography (silica, hexanes/EtOAc) to afford the tilte compound with (1R,2S)-configuration (80 mg, J_{1,2}=4.9 Hz, [MH]⁺=449/451) and the tilte compound with (1S,2S)-configutation (76 mg, J_{1,2}=6.2 Hz, [MH]⁺=449/451) as colourless solids.

### Example 2606

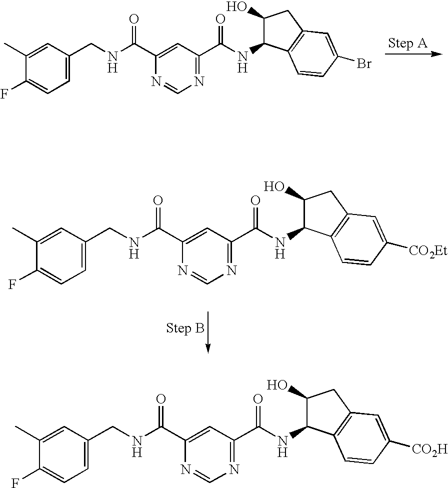 Step A

The title compound from Example 2605 (1R,2S) (8.8 mg), Pd(PPh₃)₄ (2.0 mg) and triethylamine (24 μL) were added to ethanol (1 mL) and DMSO (1 mL). The mixture was stirred for 15 h under carbon monoxide (1 atm) and diluted with ethyl acetate. The mixture was washed with brine, concentrated and purified by column chromatography (silica, hexanes/EtOAc) to afford the title compound (8.0 mg) as a colourless solid. [MH]⁺=499.

Step B

The title compound from Step A above (8.0 mg) was added to a 1M aqueous sodium hydroxide solution (2 mL) and THF (1 mL). The mixture was stirred at room temperature for 15 h and acidified to pH 2 with 1M aqueous hydrochloric acid and extracted with CH₂Cl₂ twice. The combined organic layers were dried over MgSO₄, concentrated and purified by column chromatography (silica) to afford the title compound (4.1 mg) as a colourless solid. [MH]⁺=465.

### Example 2607

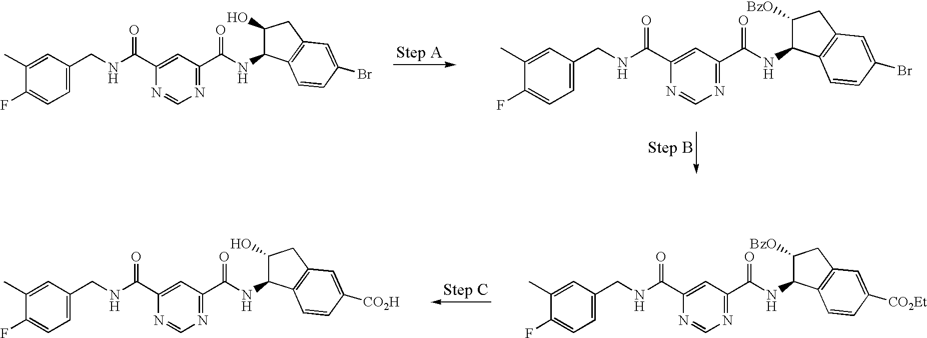 Step A

The title compound from Example 2605 (1R,2S) (80 mg), DIAD (39 mg), triphenylphosphine (63 mg) and benzoic acid (29 mg) were added to THF (3 mL). The mixture was stirred at room temperature for 24 h, concentrated and purified by column chromatography (silica, hexanes/EtOAc) to afford the intermediate (85 mg) as a colourless solid. [MNa]⁺=625/627.

Step B

The intermediate from Step A above (40 mg), Pd(PPh₃)₄ (10 mg) and triethylamine (120 μL) were added to ethanol (2 mL) and DMSO (2 mL). The mixture was stirred for 15 h under carbon monoxide (1 atm) and diluted with ethyl acetate. The mixture was washed with brine, concentrated and purified by column chromatography (silica, hexanes/EtOAc) to afford the intermediate (31 mg) as a colourless solid. [MH]⁺=597/599.

Step C

The intermediate from Step B above (5.4 mg) was added to a 1M aqueous sodium hydroxide solution (2 mL) and THF (1 mL). The mixture was stirred at room temperature for 15 h and acidified to pH 2 with 1M aqueous hydrochloric acid and extracted with CH₂Cl₂ twice. The combined organic layers were dried over MgSO₄, concentrated and purified by column chromatography (silica) to afford the title compound (3.1 mg) as a colourless solid. [MH]⁺=465/467.

### Example 2608

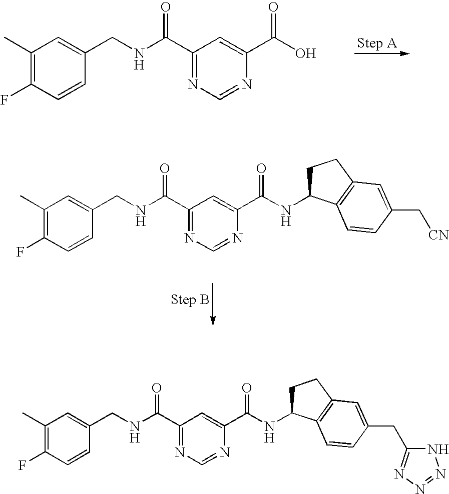 Step A

The intermediate from the Preparative Example 2112, Step E above (90 mg), the intermediate from Preparative Example 2120, Step B (124 mg), 1-(3-dimethylaminopropyl)-3-carbodiimide hydrochloride (100 mg) and 1-hydroxy-benzotriazole (70 mg) were dissolved in N,N-dimethylformamide (10 mL). After addition of N-methylmorpholine (240 μL) the reaction mixture was stirred overnight. The solvent was evaporated and the resulting residue was purified by column chromatography (silica, dichloromethane/acetone 95:5) to afford the title compound (127 mg; 67%). [MH]⁺=444.

Step B

To a solution of the title compound from Step A above (50 mg) in dry toluene (5 mL) was added dibutyltin(IV) oxide (5 mg) and trimethylsilyl azide (130 μL). The resulting mixture was heated to reflux for 19 h. The mixture was cooled to room temperature and methanol (5 mL) was added. Concentration and purification by flash chromatography (silica, chloroform/methanol, 85:15) afforded the title compound (53 mg; 99%). [MNa]⁺=509.

### Example 2609

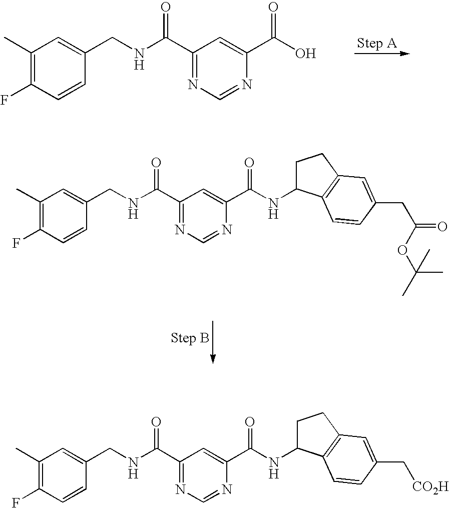 Step A

The intermediate from the Preparative Example 2123, Step D above (38.2 mg), the intermediate from Preparative Example 2120, Step B (43 mg), PyBroP (75 mg) were dissolved in N,N-dimethylformamide (3 mL). After addition of N-methylmorpholine (40 μL), the reaction mixture was stirred overnight. The solvent was evaporated and the resulting residue was purified by column chromatography (silica, cyclohexane/ethyl acetate 7:3 to 6:4) to afford the title compound (50.7 mg; 72%) as an oil. [MH]⁺=519.

Step B

To a solution of the title compound from Step A above (42.8 mg) in trifluoroacetic acid (3 mL) was added water (3 drops). The resulting mixture was stirred for 3 h and then absorbed on silica. Purification by flash chromatography (silica, dichloromethane/methanol, 95:5) afforded the title compound (35.2 mg; 92%) as a colourless foam. [MNa]⁺=463.

### Example 2610

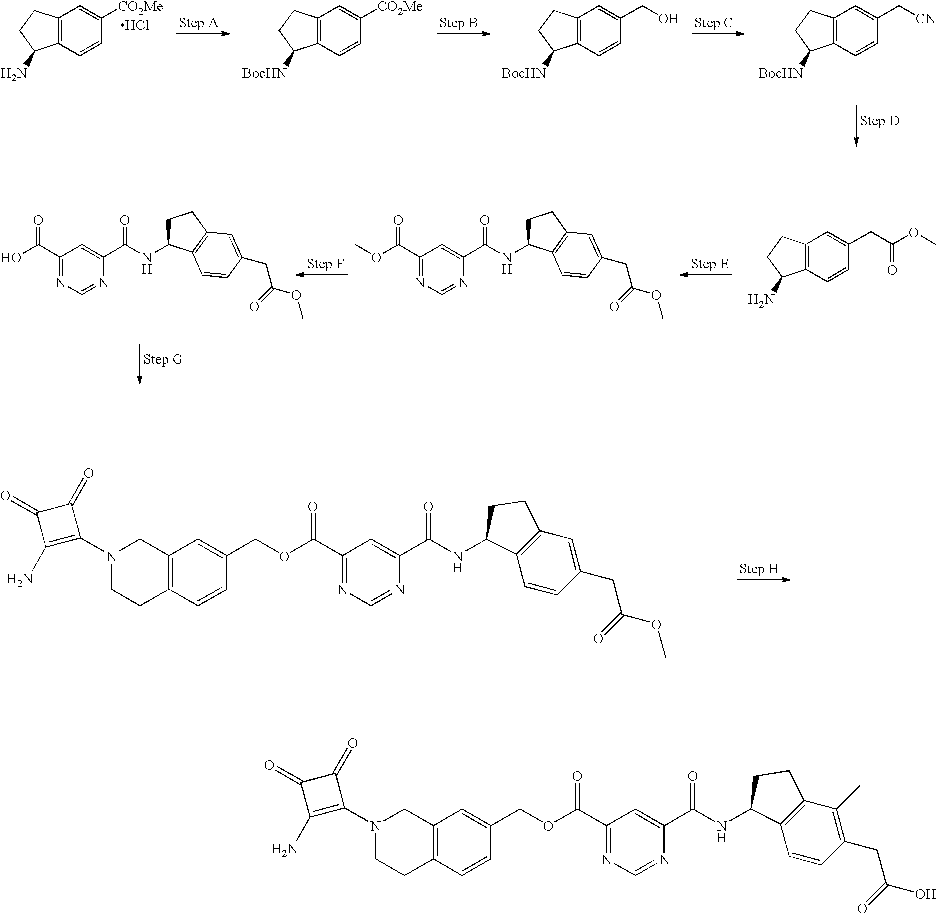 Step A

The hydrochloric acid salt of the intermediate from Preparative Example 2105, step B (450 mg) was mixed in dry CH₂Cl₂ (30 mL) and cooled to 0° C. and to this cooled solution was added di-tert-butyl dicarbonate (480 mg) followed by Et₃N (0.3 mL). After stirring for 3 h, the mixture was washed with saturated NaHCO₃ (100 mL) and brine (100 mL). The organic layer was dried over anhydrous MgSO₄, filtered, and concentrated to afford the title compound (560 mg; 96%) as a colourless solid. [MNa]⁺=314.

Step B

To a solution of the title compound from Step A above (560 mg) in dichloromethane (30 mL) was added a 1M solution of di-isobutyl-aluminiumhydride (15 mL) at 0° C. The mixture was stirred overnight an quenched with methanol. After adding Rochelle's salt, the mixture was stirred for additional 2 h. Extraction with ethyl acetate, drying (MgSO₄) and concentration of the organic layer affords the title compound (820 mg; 83%) [MNa]⁺=286.

Step C

The intermediate from Step B above (420 mg) was dissolved in dry CH₂Cl₂ (20 mL) and cooled to 0° C. and to this cooled solution was added Et₃N (0.45 mL) followed by methanesulfonyl chloride (0.25 mL). After stirring for 3 h, the mixture was diluted with dichloromethane and washed with saturated NH₄Cl (100 mL) and brine (100 mL). The organic layer was dried over anhydrous MgSO₄, filtered and concentrated to afford the intermediate as a colourless solid which was dissolved in N,N-dimethylacetamide (20 mL). After adding sodium cyanide (400 mg) the mixture was stirred at 70° C. overnight. Diethylether (80 mL) and brine (100 mL) were added and the organic layer was separated, dried (MgSO₄), filtered and concentrated and purified by chromatography (silica, dichloromethane/acetone) to afford the title compound (327 mg; 75%). [MNa]⁺=295.

Step D

The intermediate from Step C above (210 mg) was suspended in 6N hydrochloric acid (20 mL) and heated to 100° C. for 12 h upon which the solution become homogeneous. The solvent was removed under reduce pressure to give a colourless solid which was redissolved in methanol (20 mL) and cooled to 0° C. and anhydrous hydrogen chloride was bubbled through this solution for 10 min. The reaction mixture was then heated to reflux for 12 h. After cooling to room temperature, the solvent was removed under reduced pressure to give the title compound (145 mg; 92%) as a colourless solid. [M-NH₃Cl]⁺=189.

Step E

To a solution of the title compound from Step D above (90 mg) in dry dimethylformamide (5 mL) was added bromotrispyrrolidinophosphonium hexafluorophosphate (246 mg), the intermediate from Preparative Example 2117, step A (310 mg) and N-methylmorpholine (0.5 mL). The mixture was stirred at room temperature overnight and concentrated to dryness. The resdue was dissolved in water and extracted with ethyl acetate. After drying (MgSO₄) the solution was concentrated and purified by chromatography (silica, dichloromethane/acetone) to afford the title compound (285 mg; 48%) as a colourless solid. [MH]⁺=370.

Step F

The title compound from Step E above (51 mg) was dissolved in a 0.5M solution of sodium hydroxide in dry methanol (0.3 mL). The reaction mixture was stirred at room temperature for 1 h and than concentrated to afford a beige solid. This material was dissolved in water (6.2 mL) and treated with a 1M aqueous solution of hydrochloric acid (2 mL). The resulting suspension was diluted with water and extracted with ethyl acetate. After drying (MgSO₄) the solution was concentrated to afford the title compound as a colourless solid (40 mg; 82%). [MNa]⁺=378.

Step G

To a solution of the title compound from Step F above (40 mg) in dry dimethylformamide (5 mL) was added bromotrispyrrolidinophosphonium hexafluorophosphate (34 mg), the product from Preparative Example 2043, Step C (38 mg) and N-methylmorpholine (0.06 mL). The mixture was stirred at room temperature overnight and concentrated to dryness. The residue was dissolved in water and extracted with ethyl acetate. After drying (MgSO₄) the solution was concentrated to afford the crude title compound as a colourless solid which was used without further purification. [MH]⁺=595.

Step H

The crude intermediate from Step G above was dissolved in tetrahydrofurane (5 mL) and a 1M aqueous solution of lithium hydroxide was added. The reaction mixture was then stirred at room temperature (4 h), concentrated and purified by chromatography (dichloromethane/methanol 9:1) to afford the title compound (5 mg, 13% over two steps) as a colourless solid. [MH]⁺=581.

### Example 2700

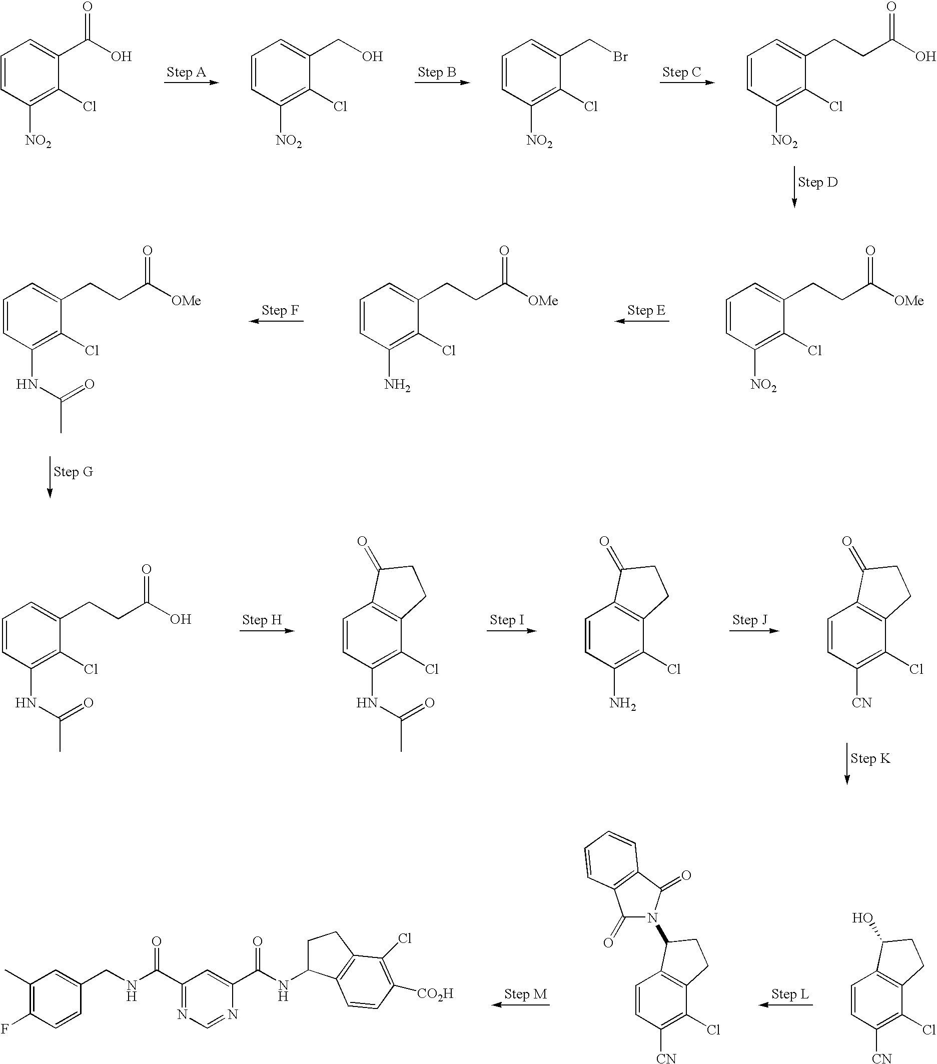 Step A

2-Chloro-3-nitro-benzoic acid (1.24 g) was dissolved in anhydrous THF (7.5 mL) under nitrogen and the reaction vessel was cooled to 0° C. in an ice bath. To this cooled solution was added a BH₃-THF complex (1M in THF, 11.2 mL) dropwise over a 1 h period. Once gas evolution had subsided, the reaction mixture was warmed to room temperature and stirred for an additional 12 h. The mixture was then poured into 1N hydrochloric acid (50 mL) cooled with ice and then extracted with Et₂O (3×15 mL). The organic extracts were combined, dried over anhydrous MgSO₄, filtered, and then concentrated to afford the intermediate (1.15 g; >99%) as a colourless solid. ¹H-NMR (CDCl₃) δ=4.90 (s, 2 H), 7.48 (t, 1 H), 7.76 (d, 1 H), 7.82 (d, 1 H).

Step B

The intermediate from Step A above (1.15 g) was dissolved in anhydrous CH₂Cl₂ (20 mL) under nitrogen and the reaction vessel was cooled to 0° C. in an ice bath. To this cooled solution was added PBr₃ (390 μL) over a 10 min period. Once the addition was complete, the reaction mixture was warmed to room temperature and stirred for an additional 2 h. The mixture was cooled in an ice bath and quenched by dropwise addition of MeOH (1 mL). The organic phase was washed with saturated NaHCO₃ (2×15 mL), dried over anhydrous MgSO₄, filtered, and then concentrated to afford the intermediate (1.35 g; 88%) as viscous oil. ¹H-NMR (CDCl₃) δ=4.66 (s, 2 H), 7.42 (t, 1 H), 7.70 (d, 1 H), 7.78 (d, 1 H).

Step C

To a mixture of NaH (60% in oil, 475 mg) in THF (30 mL) was added dimethyl malonate (1.24 mL) dropwise over 10 min. The mixture was stirred at 60° C. for 1 h and allowed to cool to 22° C. at which point a solution of the intermediate from Step B above (1.35 g) in THF (20 mL) was added dropwise over 20 min and the resulting mixture was stirred for 1.5 h. 10% H₂SO₄ (50 mL) was added and the aqueous layer was washed with Et₂O (3×50 mL). The combined organic layers were dried over anhydrous MgSO₄, filtered, and then concentrated to afford an oil. The oil was brought up in 10% NaOH (30 mL) and stirred at reflux (110° C.) for 18 h. The aqueous layer was washed with Et₂O (3×15 mL) and the organic layers were discarded. The aqueous layer was acidified with conc. HCl (10 mL) and then washed with Et₂O (3×20 mL). The combined organic layers were dried over anhydrous MgSO₄, filtered, and then concentrated to afford an oil. The resulting oil was stirred with H₂SO₄ (0.9 mL), H₂O (4.5 mL) and AcOH (6.4 mL) at 120° C. for 18 h. The reaction was allowed to cool to 22° C. and diluted with water (20 mL) and the resulting aqueous layer was washed with EtOAc (3×20 mL) and the combined organic layers were washed with brine (20 mL) and dried over anhydrous MgSO₄, filtered, and then concentrated to afford the intermediate (1.21 g; 93%) as an oil. [MH]⁺=230.

Step D

A solution of the intermediate from Step C above (1.21 g) and acetyl chloride (355 μL) in methanol (50 mL) was stirred in a sealed vessel at 65° C. for 18 h and then concentrated to afford the intermediate (1.28 g; >99%) as an oil. [MH]⁺=244.

Step E

A mixture of intermediate from Step D above (1.28 g) and iron powder (325 mesh, 724 mg) in EtOH (7 mL) and AcOH (7 mL) was stirred at 90° C. for 30 min. The mixture was filtered through Celite® and concentrated. The resulting mixture was mixed with a saturated solution of Na₂CO₃ (30 mL) and EtOAc (30 mL) for 30 min and then filtered through Celite®. The layers were separated and the aqueous layer was washed with EtOAc (30 mL). The combined organic layers were dried over anhydrous MgSO₄, filtered, and then concentrated to afford the intermediate (1.07 g; >99%) as a clear oil. [MH]⁺=214.

Step F

To a solution of intermediate from Step E above (1.07 g) and triethylamine (767 μL) in CH₂Cl₂ (30 mL) was added acetyl chloride (393 μL). The solution was stirred for 3 h and was concentrated and purified by silica gel chromatography (hexanes/EtOAc, 4:1) to afford the intermediate (800 mg; 63%). [MH]⁺=256.1.

Step G

To a solution of intermediate from Step F above (800 mg) in CH₂Cl₂ (20 mL) was added BBr₃ (650 μL). The resulting solution stirred for 24 h at 22° C. and 1N hydrochloric acid (30 mL) was cautiously added. The aqueous layer was washed with CH₂Cl₂ (2×20 mL) and the combined organic layers were dried over anhydrous MgSO₄, filtered, and then concentrated to afford the intermediate (704 mg; 99%). [MH]⁺=242.

Step H

A mixture of intermediate from Step G above (611 mg), Na₂CO₃ (268 mg), and thionyl chloride (368 μL) in CH₂Cl₂ (15 mL) under an atmosphere of nitrogen was stirred for 6 h. The mixture was filtered and the supernatant was concentrated to afford an off-white solid. The solid was dissolved in CH₂Cl₂ (15 mL) and to this solution was added AlCl₃ (675 mg). The resulting mixture was stirred at reflux (45° C.) for 18 h and then poured onto ice (40 g) and allowed to warm to 22° C. The layers were separated and the aqueous layer was washed with CH₂Cl₂ (2×30 mL). The organic layers were combined, dried over anhydrous MgSO₄, filtered, concentrated, and purified by silica gel chromatography (hexanes/EtOAc, 1:1) to afford the intermediate (377.5 mg; 67%) as an off-white solid. [MH]⁺=224.

Step I

A mixture of intermediate from Step H above (377.5 mg) in 3N aqueous LiOH (3 mL), THF (6 mL), and MeOH (6 mL) was stirred at 50° C. for 1 h. The resulting solution was concentrated and diluted with water (15 mL) and washed with CH₂Cl₂ (3×15 mL). The combined organic layers were dried over anhydrous MgSO₄, filtered and concentrated to give the intermediate (284 mg; 93%). [MH]⁺=182.

Step J

At 0° C. was added dropwise over 5 min a solution of NaNO₂ (42 mg) in water (1 mL) to a mixture of intermediate from Step I above (106 mg) in 2N hydrochloric acid (2 mL). The mixture was stirred at 0° C. for 15 min at which time all solids had dissolved. Solid Na₂CO₃ (250 mg) was cautiously added, which caused the mixture to turn a dark red. The mixture was pipetted into a solution of CuCN, which had been premixed by stirring CuCl (72 mg) and NaCN (92 mg) in water (2 mL) for 1 h. Once the reddish mixture had been pipetted into the CuCN solution the resulting mixture was stirred at 0° C. for 1 h and then allowed to warm to 22° C. over 30 min and then heated to 50° C. for 15 min. Saturated NaHCO₃ (10 mL) was added and the resulting aqueous layer was washed with EtOAc (3×10 mL). The combined organic layers were dried over anhydrous MgSO₄, filtered, concentrated, and purified by silica gel chromatography (hexanes/EtOAc, 3:1) to afford the intermediate (50 mg; 43%) as an off-white solid. [MH]⁺=191.9. ¹H-NMR (CDCl₃) δ=2.81 (dd, 2 H), 3.22 (dd, 2 H), 7.76 (m, 2 H).

Step K

To a cooled solution of (S)-2-methyl-CBS-oxazaborolidine (1M in toluene, 700 μL) and borane-methyl sulfide complex (1M in CH₂Cl₂, 700 μL) at −20° C. (internal temperature) was added a solution of intermediate from Step J above (133 mg, in 1 mL CH₂Cl₂) over a 1.5 h period using a syringe pump. After the addition was completed, the mixture was quenched by addition of MeOH (1 mL) at −20° C., warmed to room temperature and concentrated. The crude mixture was purified by silica gel chromatography (hexanes/EtOAc, 3:1) to afford the intermediate (98.5 mg; 73%) as a colourless solid. [MH]⁺=194.

Step L

To a solution of intermediate from Step K above (14 mg), PPh₃ (26.6 mg), and phthalimide (15 mg) in THF (800 μL) at 0° C. was added diisopropyl azodicarboxylate (20 μL). The reaction solution was allowed to warm to 22° C. and stirred for 2 h and then concentrated and purified by silica gel chromatography (hexanes/EtOAc, 5:1) to afford the intermediate (16 mg; 69%) as a colourless solid. [MH]⁺=323.

Step M

A solution of intermediate from Step L above (32 mg) and hydrazine (55% in water, 17 μL) in EtOH (I mL) was stirred for 4 h and then concentrated to a colourless solid. The solid was mixed with conc. HCl (5 mL) and stirred at 105° C. for 48 h and then concentrated to a colourless solid. To this solid was added a solution of HCl in MeOH (5 mL, bubbled anhydrous hydrogen chloride through MeOH for 5 min) and the mixture was stirred at 65° C. in a sealed vessel for 18 h. The solution was concentrated to a white solid and analysis revealed a mixture of 4-chloro-5-cyano-indan-1-yl-ammonium chloride and 4-chloro-5-methoxycarbonyl-indan-1-yl-ammonium chloride, which were separated in the final step. To a solution of the intermediate from the Preparative Example 2120, Step B (43 mg) and N,N-dimethylformamide (5 μL) in CH₂Cl₂ (1.5 mL) at 0° C. was added oxalyl chloride (26 μL). The solution was allowed to warm to 22° C. and stirred for 2 h. The solution was concentrated and the resulting residue was dissolved in CH₂Cl₂ (1.5 mL). The resulting solution was canulated into the above mentioned mixture of 4-chloro-5-cyano-indan-1-yl-ammonium chloride and 4-chloro-5-methoxycarbonyl-indan-1-yl-ammonium chloride and triethylamine (56 μL) in CH₂Cl₂ (1.5 mL) and the mixture was stirred for 2 h at which time it was a homogeneous solution. Silica gel (500 mg) was added and the mixture was concentrated and purified by silica gel chromatography (hexanes/ethyl acetate, 1:1) to afford an off-white solid of a mixture of pyrimidine-4,6-dicarboxylic acid 4-[(4-chloro-5-cyano-indan-1-yl)-amide]6-(4-fluoro-3-methyl-benzylamide) and 4-chloro-1-{[6-(4-fluoro-3-methyl-benzylcarbamoyl)-pyrimidine-4-carbonyl]-amino}-indan-5-carboxylic acid methyl ester. This mixture was dissolved in THF (200 μL), MeOH (200 μL) and 3N aqueous LiOH (100 μL) and was stirred at 50° C. for 1 h. The solution was concentrated to remove all methanol and the resulting residue was dissolved in THF (200 μL) and acidified with concentrated hydrochloric acid (30 μL). The mixture was concentrated and purified by silica gel chromatography (hexanes/EtOAc, 1:1 to remove the pyrimidine-4,6-dicarboxylic acid 4-[(4-chloro-5-cyano-indan-1-yl)-amide]6-(4-fluoro-3-methyl-benzylamide) and then CH₂Cl₂/MeOH, 9:1) to afford an off-white solid of the title compound (15.0 mg; 31%). [MH]⁺=483.

### Example 2701

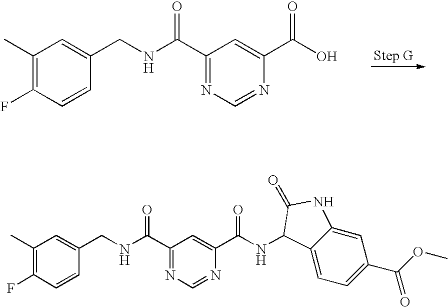 Step A

The intermediate from the Preparative Example 2109, Step F (250 mg) and carbonyldiimidazole (140 mg) were dissolved in DMF (5 mL) and stirred for 1 h. The intermediate from the Preparative Example 2120, Step B (210 mg) was dissolved in DMF (3 mL) and triethylamine (105 mg) was added. The resulting mixture was transferred by pipette to the acid solution and stirred 1 h. Volatiles were removed under reduced pressure and the crude product taken up in ethyl acetate and dry packed on silica. Purification by column chromatography (10% methanol in dichloromethane) resulted in the isolation of the title compound (175 mg) as a pale orange solid. [MH]⁺=478.

### Example 2702

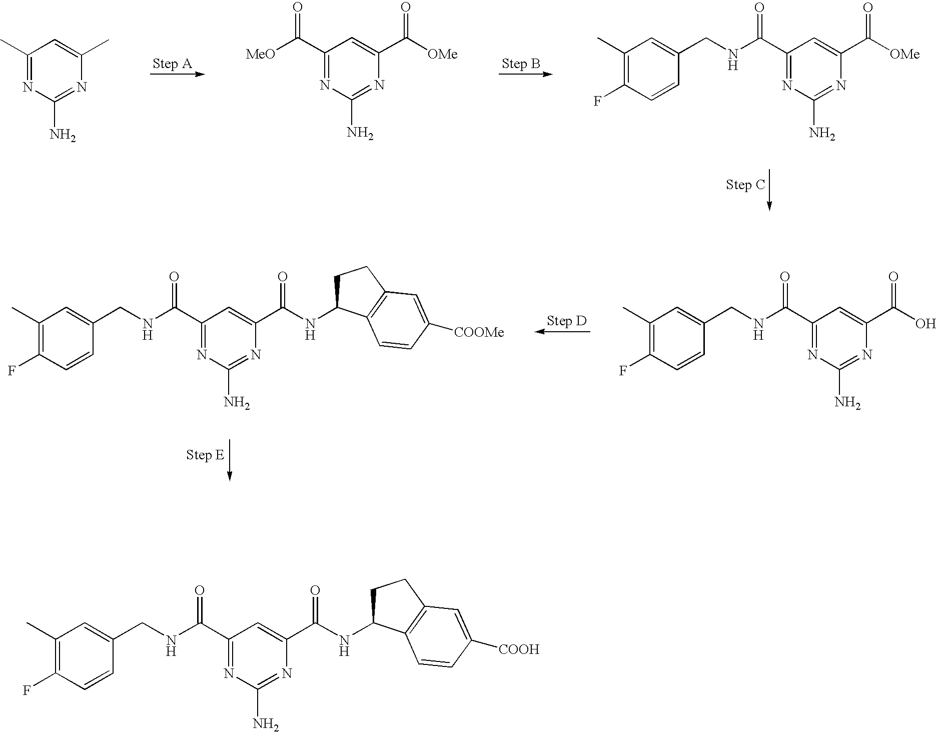 Step A

To commercially available 4,6-dimethyl-pyrimidin-2-ylamine (6.0 g) in water (400 mL) was added a solution of sodium hydroxide (1.3 g in 5 mL water) and heated at 80° C. for 10 min. Then potassium permanganate (15 g) was added and heated between 85° C. to 90° C. for 1 h. Potassium permanganate (15 g) was again added and mixture was heated for another 2 h. The mixture was cooled to room temperature and filtered through Celite® and then acidified to pH ˜2. The mixture was concentrated to 20% of the original volume and the solid was filtered and dried. To solid was dissolved in methanol (200 mL) and saturated with dry hydrogen chloride gas and the mixture was heated to reflux for 24 h. The mixture was concentrated to an oil and then taken up in dichloromethane and the organic phase was washed with saturated NaHCO₃ and then dried over MgSO₄, filtered and concentrated to give a solid which was purified by column chromatography (silica, 10% methanol/dichloromethane) to give the intermediate (0.41 g). [MH]⁺=212.

Step B

A solution of the intermediate from Step A above (0.24 g) in NAN-dimethylformamide (3 mL) was added 4-fluoro-3-methyl-benzylamine (0.15 g) dissolved in N,N-dimethylformamide (1 mL) and the mixture was stirred at 80° C. for 15 h, concentrated and then purified by column chromatography (silica, 10% methanol/dichloromethane) to afford the intermediate (0.15 g; 28%) as a colourless foam. [MH]⁺=319.

Step C

A solution of the intermediate of Step B above (0.15 g) in tetrahydrofurane (2 mL) was added a 1N potassium hydroxide solution (2 mL) and was stirred for 24 h. The mixture was concentrated and purified by column chromatography (silica, 10% methanou/dichloromethane) to afford the intermediate (60 mg; 42%). [MH]⁺=305.

Step D

To a solution of the intermediate of Step C above (20 mg) in N,N-dimethylformamide (0.5 mL) was added N-methylmorpholine (15 μL) and the mixture was chilled (−40° C.) under nitrogen. To the chilled solution was then added isobutyl chloroformate (10 μL) and mixture was stirred at between −40° C. to −20° C. for 1.5 h. To the chilled solution was added the intermediate from Preparative Example 2105, Step B (13 mg) dissolved in tetrahydrofurane (0.5 mL) and mixture allowed to stir at −40° C. to −20° C. for 1 h and then slowly warm to room temperature. To the mixture was then added water (1-2 drops) and stirring was continued for 1 h. The mixture was concentrated and resulting solid purified by preparative thin layer chromatography (silica, 10% methanol/dichloromethane) to give the intermediate (20 mg; 64%). [MH]⁺=478.

Step E

To the intermediate of Step D above (20 mg) dissolved in tetrahydrofurane (0.4 mL) was added a 1N potassium hydroxide solution (40 μL) and water (100 μL) and the mixture was stirred at room temperature for 15 h. The mixture was concentrated and to the resulting solid was then added 1N hydrochloric acid (0.3 mL) and then concentrated to a solid. The solid was purified by preparative thin layer chromatography (silica, 10% methanol/dichloromethane) to give the title compound (9 mg; 47%). [ME]⁺=464.

### Example 2703

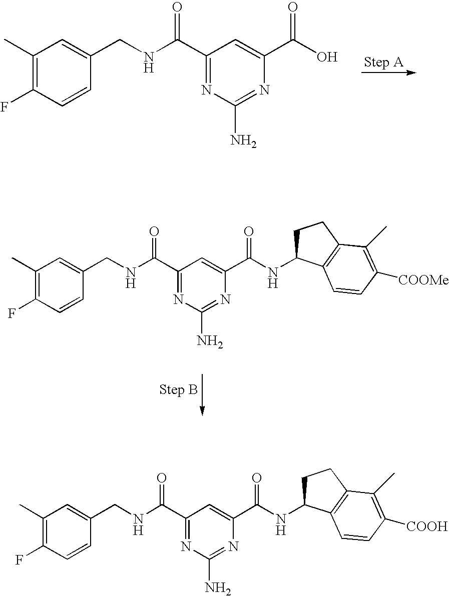 Step A

To a solution of the intermediate from Example 2702, Step C (25 mg) in N,N-dimethylformamide (0.3 mL) was added benzotriazole-1-yl-oxy-tris-pyrrolidino-phosphonium hexafluorophosphate (51 mg), the intermediate from Preparative Example 2110, Step K (22 mg) and triethylamine (50 μL) and tetrahydrofurane (0.3 mL) and the mixture was allowed to stir at room temperature for 24 h. The mixture was then concentrated and purified by preparative thin layer chromatography (silica, 10% methanol/dichloromethane) to give the intermediate (14 mg; 35%). [MH]⁺=492.

Step B

To the intermediate of Step B above (14 mg) dissolved in tetrahydrofurane (0.5 mL) was added 1N LiOH (0.5 mL) and methanol (0.3 mL) and the mixture was stirred at room temperature for 12 h. The mixture was concentrated and the resulting solid acidified with 1N hydrochloric acid and then concentrated to a solid. The solid was purified by preparative thin layer chromatography (silica, 10% methanol/dichloromethane) to give the title compound (10 mg; 73%). [MH]⁺=478.

### Example 2704

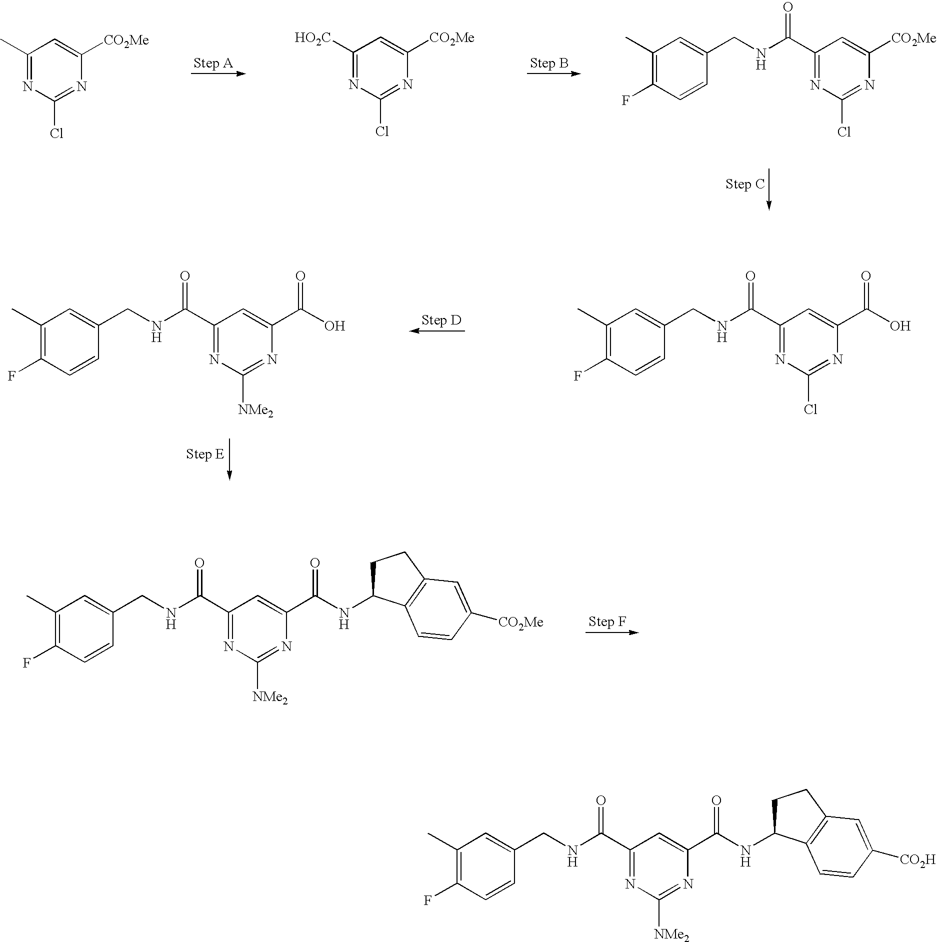 Step A

Commercially available 2-chloro-6-methyl-pyrimidine-4-carboxylic acid methyl ester (9.38 g) and selenium dioxide (8.93 g) were dissolved in dioxane (50 mL) and stirred at 105° C. in a round-bottom flask under argon. After 12 h the mixture was filtered twice through Celiteo and washed well with dioxane (2×100 mL). The filtrate was then evaporated to afford the intermediate (8.0 g; 74%) as viscous orange oil. [MH]⁺=217.

Step B

The intermediate from Step A above (0.9 g) was dissolved in dry dichloromethane (20 mL) and cooled to 0° C. Then oxalyl chloride (0.87 mL) was slowly added followed by 2-3 drops of N,N-dimethylformamide and the cooling was removed. After the gas evolution was complete, the mixture was concentrated, dissolved in dichloromethane, pyridine (0.34 mL) was added followed by 4-fluoro-3-methylbenzylamine (0.53 mL) and the reaction was stirred for 30 min. MS analysis showed the product to be present ([MH]⁺=338). The mixture was filtered and evaporated onto silica. Product was eluted with 30% ethyl acetate/hexane via column chromatography. This afforded the intermediate (0.67 g) as a yellow solid.

Step C

A solution of the intermediate from Step B above (670 mg) in tetrahydrofurane (20 mL) was cooled to 0° C. and 1M aqueous lithium hydroxide (3.98 mL) was slowly added and the reaction was stirred for 2 h at 0° C. Analysis of the reaction via MS showed the product as the acid ([MH]⁺=324). The mixture was quenched with 1M hydrochloric acid (4.0 mL) and warmed to room temperature. The mixture was reduced to dryness in vaccuo and the product extracted via trituration with tetrahydrofurane and filtration. The filtrate was evaporated to afford the intermediate (1.1 g) an orange solid.

Step D

To a solution of the intermediate from Step C above (0.1 g) in tetrahydrofurane (1 mL) was added dimethylamine (2M in tetrahydrofuran, 0.6 mL) and the mixture was stirred for 15 h. The mixture was concentrated and then acidified with 1N hydrochloric acid and then filtered. The solid was purified by column chromatography (silica, 40% diethyl ether/dichloromethane) to afford the intermediate (54 mg; 54%). [MH]⁺=333.

Step E

To a solution of the intermediate from Step D above (54 mg) in N,N-dimethylformamide (1 mL) was added benzotriazole-1-yl-oxy-tris-pyrrolidino-phosphonium hexafluorophosphate (85 mg), the intermediate from Preparative Example 2105, Step B (31 mg), triethylamine (40 μL) and dichloromethane (0.5 mL) and allowed to stir at room temperature for 24 h. The mixture was then concentrated and purified by column chromatography (silica, 30% diethyl ether/dichloromethane) to give the intermediate (70 mg; 86%). [MH]⁺=506.

Step F

To a solution of the intermediate from Step E above (70 mg) in tetrahydrofurane (0.3 mL) was added 1N aqueous sodium hydroxide (0.3 mL) and methanol (0.3 mL) and the mixture was stirred at room temperature for 24 h. The mixture was concentrated and purified by column chromatography (silica, 30% methanol/dichloromethane) to give the title compound (22 mg; 32%). [MH]⁺=492.

### Example 2705

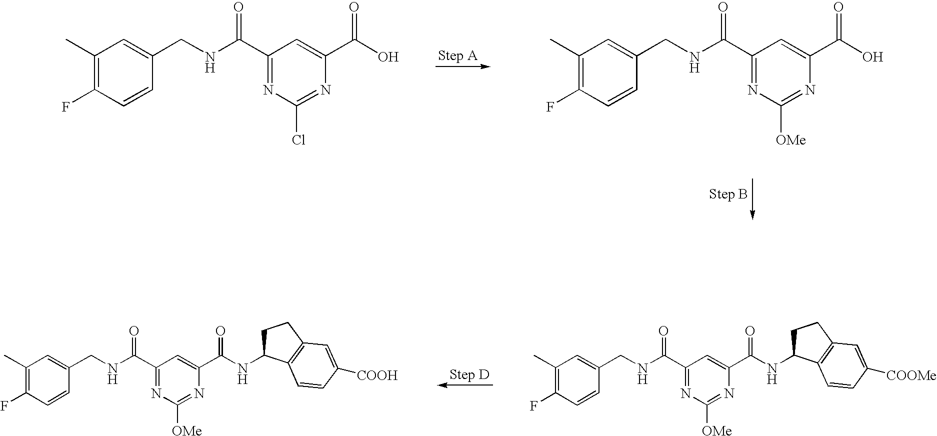 Step A

To a solution of the intermediate from Example 2704, Step C (80 mg) in tetrahydrofurane (1 mL) was added sodium methoxide (0.5M in methanol, 2 mL) and stirred for 15 h. The mixture was concentrated and then acidified with 1N hydrochloric acid and then filtered to afford the intermediate (50 mg). [MH]⁺=320.

Step B

To a solution of the intermediate from Step A above (50 mg) in N,N-dimethylformamide (1 mL) was added benzotriazole-1-yl-oxy-tris-pyrrolidino-phosphonium hexafluorophosphate (82 mg), the intermediate from Preparative Example 2105, Step B (35 mg), triethylamine (50 μL) and dichloromethane (1 mL). After stirring at room temperature for 24 h the mixture was concentrated and purified by column chromatography (silica, 30% diethyl ether/dichloromehane) to give the intermediate (40 mg; 50%). [MH]⁺=493.

Step C

To a solution of the intermediate from Step B above (40 mg) in tetrahydrofurane (0.3 mL) was added 1N aqueous sodium hydroxide (0.3 mL) and methanol (0.3 mL) and mixture was stirred at room temperature for 24 h. The mixture was concentrated and purified by column chromatography (silica, 10% methanol/dichloromethane) to give the title compound (26 mg; 67%). [MH]⁺=479.

### Example 2706

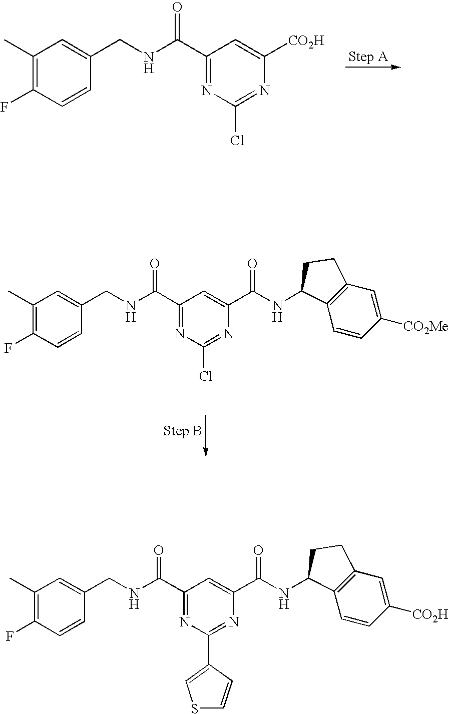 Step A

The intermediate from Example 2704, Step C (0.92 g) was dissolved in CH₂Cl₂ (20 mL) and DMF (0.2 mL) and cooled to 0° C. Oxalyl chloride (0.81 mL) was added dropwise. After stirring for 1 h, gas evolution subsided and a solution of the intermediate from Preparative Example 2105, Step B (0.60 g) and triethylamine (0.44 mL) in CH₂Cl₂ (5 mL) was added dropwise. After stirring at room temperature for 3 h, the mixture was concentrated under high vacuum to give crude product which was purified by flash chromatography using 20% EtOAc/CH₂Cl₂ to give the intermediate (0.95 g; 67%) as a colourless solid. [MH]⁺=497.

Step B

The intermediate from Step A above (50 mg) was dissolved in dimethoxyethane (5 mL) under nitrogen with 0.4N aqueous Na₂CO₃ (0.50 mL), 3-thiophenyl boronic acid (14 mg) and tetrakis triphenylphosinepalladium(0) (12 mg). After heating the reaction mixture to 100° C. stirring for 8 h, LC/MS showed the complete disappearance of starting material. After cooling to room temperature, the mixture was concentrated under high vacuum to give crude product which was purified by flash chromatography using 25% MeOH/CH₂Cl₂ to give the title compound (47 mg; 53%) as a colourless solid. [MH]⁺=531.

### Examples 2707-2709

Following a similar procedure as that described in Example 2706, Step B, except using the boronic acid indicated in Table 23 below, the following compounds were prepared.

TABLE 23

| | | | Yield |
|---|---|---|---|
| Ex. # | Boronic acid | Product | MS |
| 2707 | 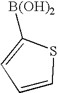 | 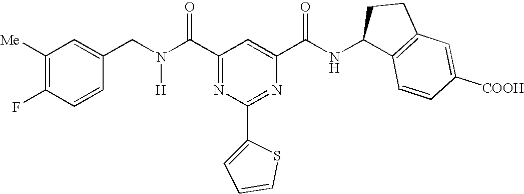 | 22% [MH]⁺ = 531 |
| 2708 | 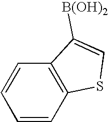 | 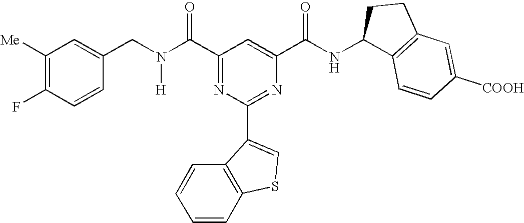 | 26% [MH]⁺ = 581 |
| 2709 | 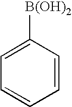 | 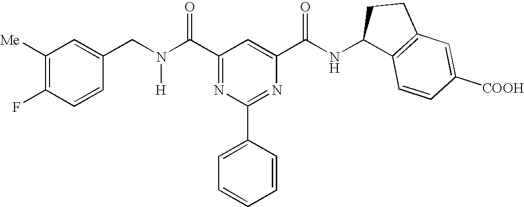 | 42% [MH]⁺ = 525 |

### Example 2710

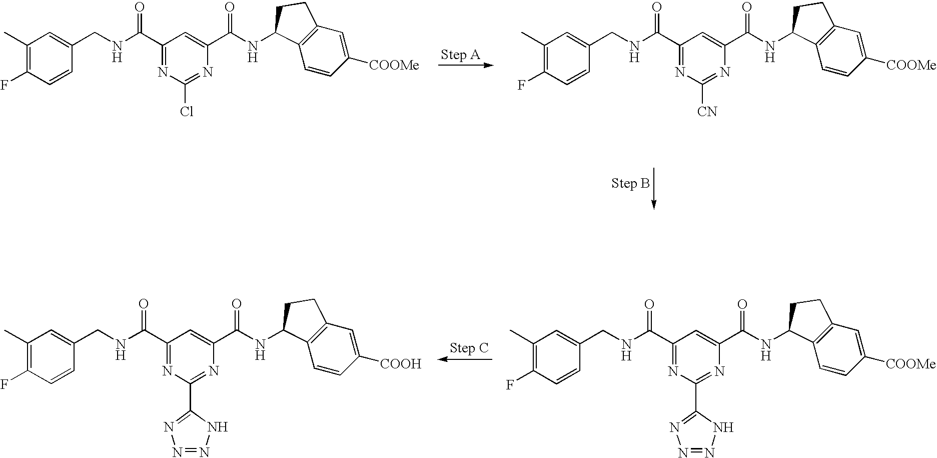 Step A

To a solution of the intermediate from Example 2706, Step A (0.24 g), Zn(CN)₂ (112 mg) and Pd(PPh₃)₄ (139 mg) were combined under nitrogen and anhydrous DMF (5 mL) was added. The yellow mixture was heated to 105° C. for 18 h and then concentrated. The mixture was purified by column chromatography (30% diethyl ether/dichloromethane) to give the intermediate (0.15 g; 64%). [M-H]⁻=486.

Step B

To the solution of the intermediate from Step A above (50 mg) in anhydrous toluene (1 mL) was added dibutyltinoxide (11 mg) and azidotrimethylsilane (55 μL) and the mixture was heated to 105° C. for 3 h and then concentrated. The residue was purified by column chromatography (30% diethyl ether/dichloromethane) to give the intermediate (50 mg; 92%). [M-H]⁻=529.

Step C

To the a solution of the intermediate from Step B above (50 mg) in tetrahydrofurane (1 mL) was added 1N aqueous sodium hydroxide (0.5 mL) and methanol (0.3 mL) and the mixture was stirred at room temperature for 24 h. The mixture was concentrated and purified by preprative thin layer chromatography (silica, 20% methanol/dichloromethane) to give the title compound (25 mg; 51%). [M-H]⁻=515.

### Example 2711

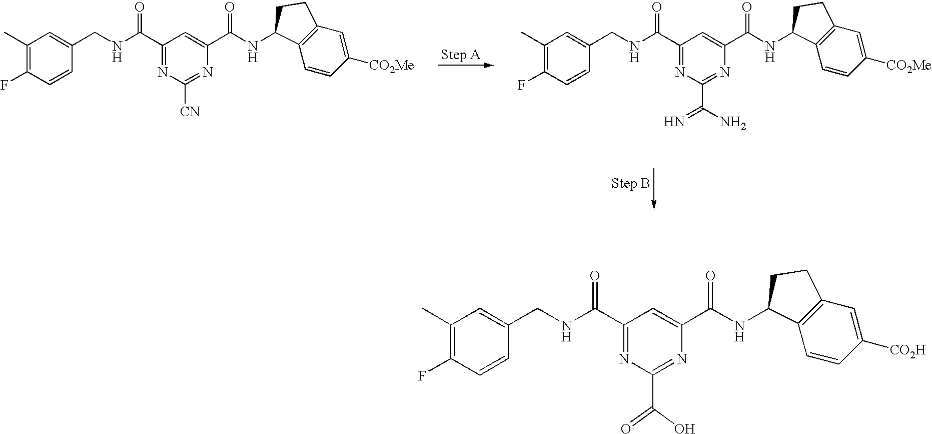 Step A

The intermediate from the Example 2710, Step A (25 mg) was dissolved in anhydrous MeOH (20 mL) and cooled in an ice bath upon which anhydrous hydrogen chloride gas was bubbled through for 1 min. The reaction mixture was then sealed and placed in a refrigerator (4° C.) overnight. The mixture was warmed to room temperature and concentrated to give a pale, colourless oil to which was added ammonia (6N in MeOH, 5 mL) and this mixture was stirred at room temperature for 10 h. After evaporation under high vacuum, the crude product was purified by flash chromatography using 5% MeOH/CH₂Cl₂ to give the intermediate (15 mg; 53%) as a colourless solid. [MH]⁺=505.

Step B

The title compound from Step A above (15 mg) was dissolved in THF (2 mL) and MeOH (2 mL) with lithium hydroxide (20 mg) and heated to 50° C. for 5 h. The reaction mixture was then concentrated under high vacuum to afford crude product which was collected and washed with water (3×3 mL) and dried to give the title compound (10 mg; 68%) as a colourless solid. [MH]⁺=493.

### Example 2712

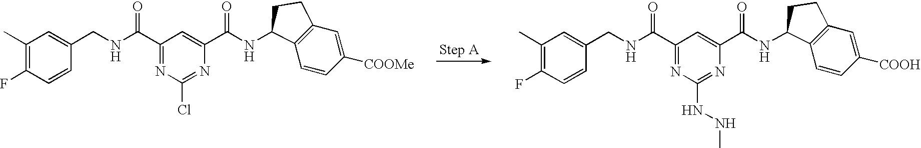 Step A

The intermediate from Example 2706, Step A (50 mg), methylhydrazine (5 mg) and triethylamine (12 mg) was heated in DMF (0.25 mL) at 40° C. for 1 h. The mixture was diluted with ethyl acetate and washed with water. The crude product was purified by column chromatography (5% methanol in dichloromethane) and saponified (2 mL THF/MeOH 1:1, 0.33 mL 1N NaOH) overnight. The resulting acid was purified by chromatography (10% methanol in dichloromethane) to give the title compound (20 mg; 40%) as a colourless solid. [MH]⁺=493.

### Example 2713

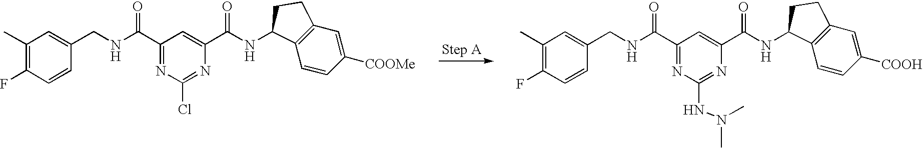 Step A

Following a similar procedure as that described in Example 2712, except using N,N-dimethylhydrazine, the title compound was obtained in 12%. [MH]⁺=507.

### Example 2714 and Example 2715

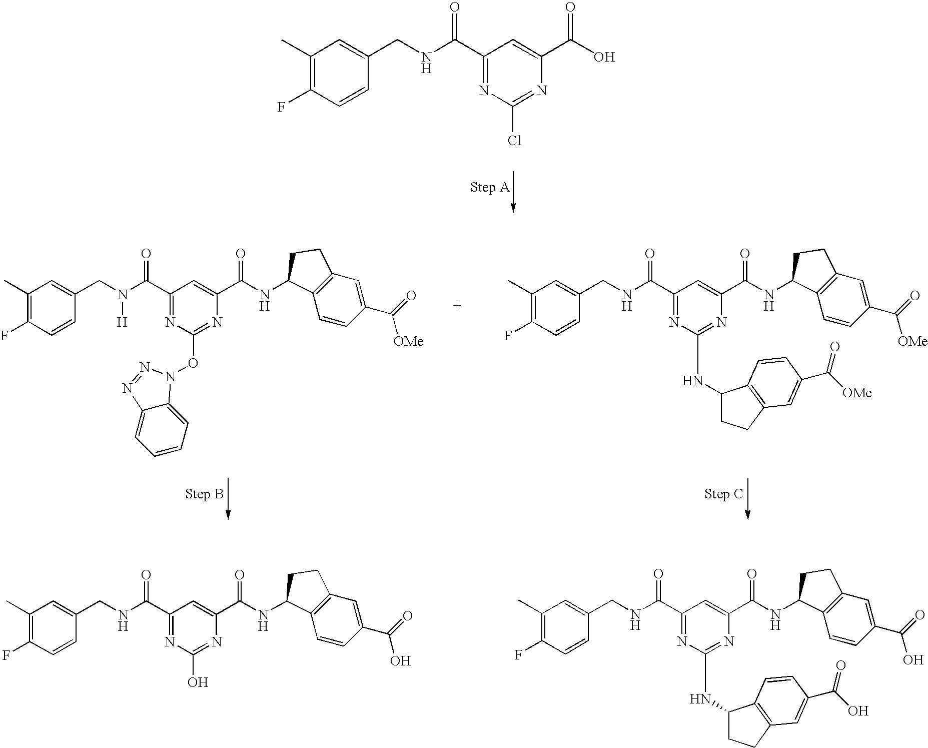 Step A

To the intermediate from Example 2704, Step C (323 mg), the intermediate from Preparative Example 2105, Step B (191 mg), triethylamine (0.35 mL) in THF (5 mL) was added PyBop (550 mg) at room temperature. The reaction mixture was stirred for 1 h and then was concentrated to dryness. The solid was dissolved in ethyl acetate (20 mL) and the resulting solution was washed with 1M hydrochloric acid (5 mL), saturated aqueous sodium bicarbonate (5 mL) and brine (5 mL). The solution was dried over magnesium sulfate and concentrated in vaccuo. The crude mixture was purified by silica gel chromatography to give two intermediates: the 2-OBt product (300 mg; 50%, [MH]⁺=596) and the 2-indanylamino product (163 mg; 28%, [MH]⁺=652).

Step B

To the first title compound from Step A above (2-OBt product) (36.5 mg) in tetrahydrofurane (1 mL) was added 1M aqueous sodium hydroxide (0.3 mL). After 1 h at 40° C., the solution was neutralized with 2M aqueous sodium bisulfate (0.3 mL). The resulting solution was concentrated to dryness. The solid was titrated with tetrahydrofurane (5 mL), dried over magnesium sulfate and concentrated in vaccuo to give the title compound (21 mg; 70%) as a colouless solid. [MH]⁺=465.

Step C

To the second title compound from Step A above (2-indanylamino product) (35 mg) in tetrahydrofurane (2 mL) was added 1M aqueous sodium hydroxide (0.16 mL) and stirred overnight. The solution was neutralized with 2M aqueous sodium bisulfate (0.2 mL). The resulting solution was concentrated to dryness. The solid was titrated with tetrahydrofurane (5 mL), dried over magnesium sulfate and concentrated in vaccuo to give the title compound (29 mg; 87%) as a colourless solid. [MH]⁺=624.

### Example 2716

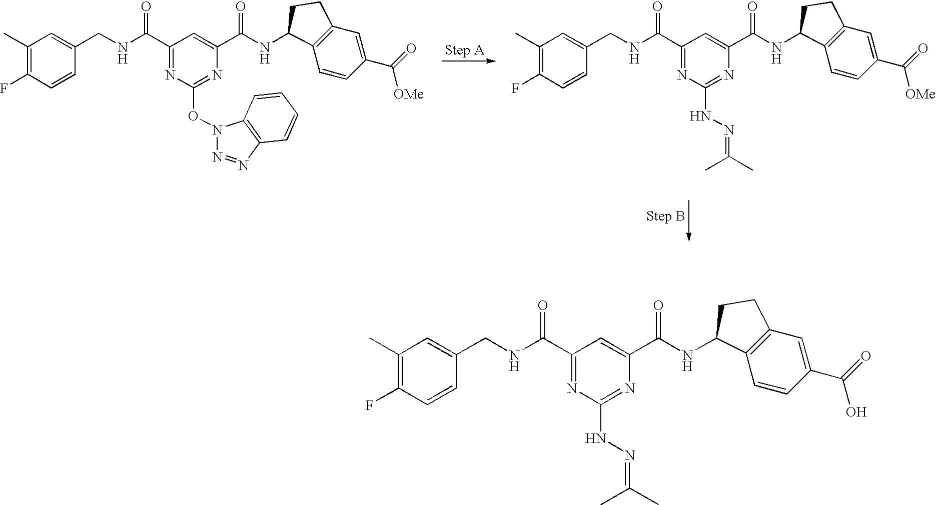 Step A

To a stirred solution of the title compound from Example 2714, Step A above (100 mg) in anhydrous THF (5 mL) was added hydrazine (1M solution in THF, 2 mL) and stirring was continued at room temperature for 2 h. The solvent was then removed in vaccuo. The crude product was purified by flash chromatography (10% acetone in dichloromethane) to afford the intermediate (77 mg; 85%). [MH]⁺=533.

Step B

A solution of the title compound from Step A above (30 mg) in MeOH (1 mL) and THF (2 mL) was treated with 1N aqueous lithium hydroxide solution (0.5 mL) and stirred overnight at room temperature. The reaction mixture was acidified to pH 4.5 with 2N hydrochloric acid and stirred for 15 min at room temperature. The mixture was extracted with EtOAc. The organic layer was washed with brine, dried over MgSO₄ and evaporated. The resulting residue was purified by column chromatography (10% methanol in dichloromethane) to afford the title compound (2.2 mg; 8%). [MH]⁺=519.

### Example 2717

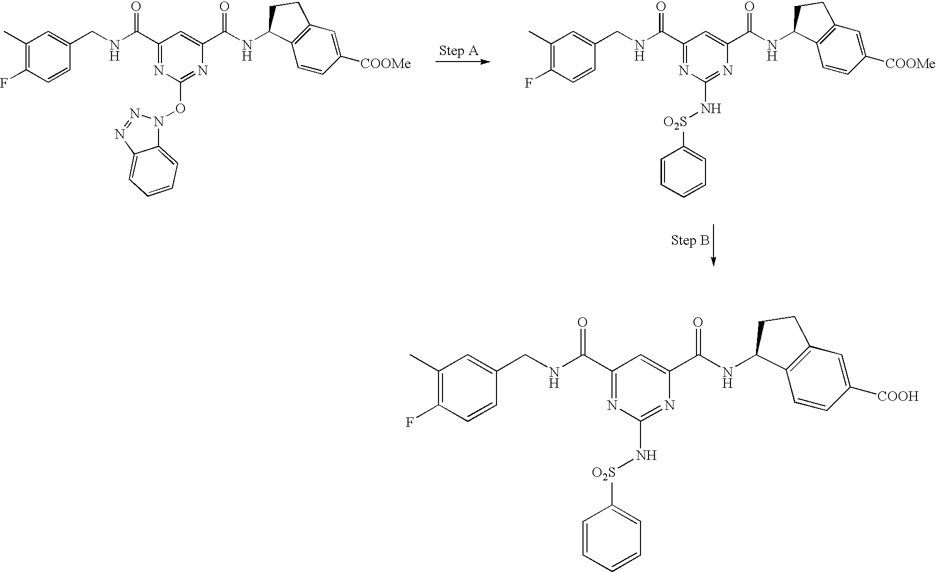 Step A

To the intermediate from from Example 2714, Step A (70 mg) in dioxane (1 mL) was added sodium tert-butoxide (14 mg) and benzene sulfonamide (24 mg) and the mixture was stirred at room temperatue for 1 h and then at 70° C. for 10 h. The mixture was concentrated and purified by column chromatography (silica, 30% diethyl ether/dichloromethane) to give the intermediate (40 mg; 55%). [MH]⁺=618.

Step B

To a solution of intermediate from Step A above (40 mg) in tetrahydrofurane (1 mL) was added 1N aqueous sodium hydroxide (0.5 mL) and methanol (0.3 mL) and the mixture was stirred at room temperature for 24 h. The mixture was concentrated and purified by preparative thin layer chromatography (silica, 15% methanol/dichloromethane) to give the title compound (26 mg; 66%). [MH]⁺=604.

### Example 2718

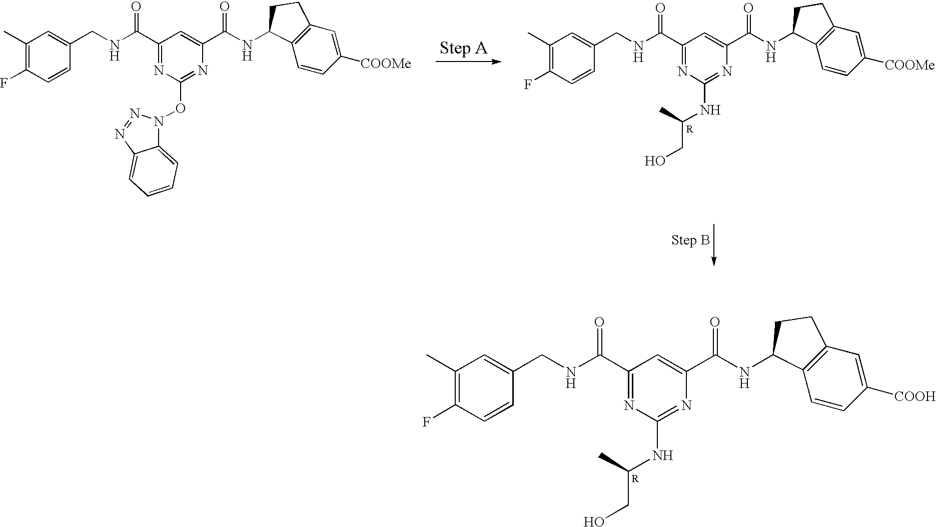 Step A

To a solution of the intermediate from from Example 2714, Step A (46 mg) in N,N-dimethylformamide (0.2 mL) and tetrahydrofurane (1 mL) was added commercially available (R)-2-amino-1-propanol (12 μL) and the mixture was stirred at room temperature for 48 h and then concentrated to give the intermediate (50 mg). [MH]⁺=536.

Step B

To a solution of intermediate from Step A above (50 mg) in tetrahydrofurane (0.3 mL) was added 1N aqueous lithium hydroxide (0.5 mL) and methanol (0.3 mL) and the mixture was stirred at room temperature for 12 h. The mixture was concentrated and acidified with IN hydrochloric acid and then concentrated again. The mixture was purified by preparative thin layer chromatography (silica, 10% methanou/dichloromethane) to give the title compound (20 mg; 50% over two steps). [MH]⁺=522.

### Example 2719

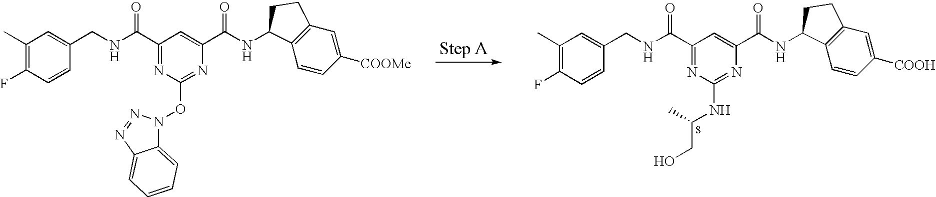 Step A

Following a similar procedure as that described in Example 2718, except using (S)-2-amino-1-propanol, the title compound was obtained in 40% over two steps. [MH]⁺=522.

### Example 2720

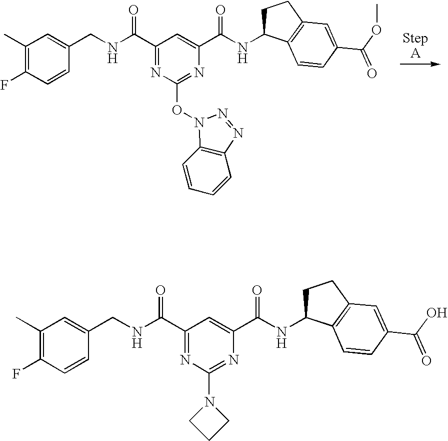 Step A

The intermediate from Example 2714, Step A (50 mg) was combined with azetidine (5 mg) under nitrogen in anhydrous THF (1 mL) and the mixture was stirred at room temperature. TLC analysis showed complete disappearance of starting material after 1 h upon which MeOH (1 mL) was added followed by NaOH (1M in H₂O, 0.5 mL). The reaction mixture was stirred at room temperature for an additional 12 h. The solvent was removed under reduced pressure and the remaining residue was partitioned between EtOAc (10 mL) and 1M hydrochloric acid (10 mL). The organic layer was dried over MgSO₄, filtered, and concentrated to give the crude product which was purified by flash chromatography using 20% MeOH/CH₂Cl₂ to give the title compound (18 mg; 43%) as a colourless solid. [MH]₊=504.

### Examples 2721-2724

Following a similar procedure as that described in Example 2720, except using the amine indicated in Table 24 below, the following compounds were prepared.

TABLE 24

| | | | Yield |
|---|---|---|---|
| Ex. # | Amine | Product | MS |
| 2721 | 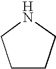 | 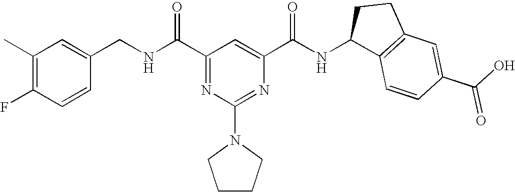 | 19% [MH]⁺ = 518 |
| 2722 | 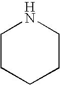 | 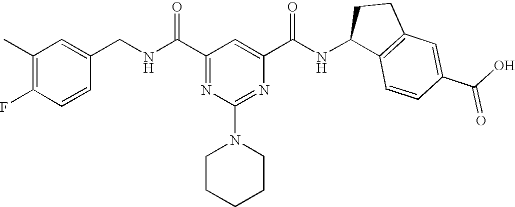 | 24% [MH]⁺ = 532 |
| 2723 | 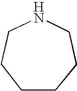 | 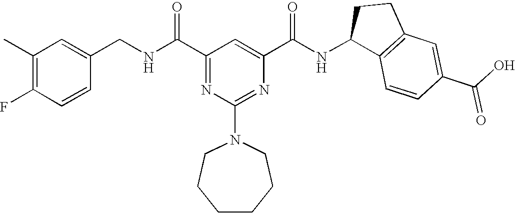 | 31% [MH]⁺ = 546 |
| 2724 | 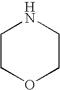 | 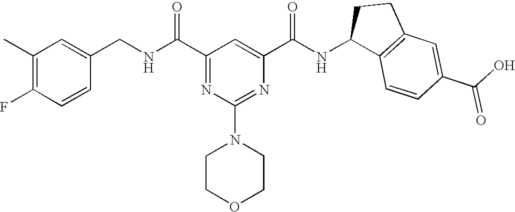 | 24% [MH]⁺ = 534 |

### Example 3000

### Assay for Determining MMP-13 Inhibition

The typical assay for MMP-13 activity is carried out in assay buffer comprised of 50 mM Tris, pH 7.5, 150 mM NaCl, 5 mM CaCl₂ and 0.05% Brij-35. Different concentrations of tested compounds are prepared in assay buffer in 50 μL aliquots. 10 μL of 40 nM stock solution of MMP-13 enzyme is added to the compound solution. The mixture of enzyme and compound in assay buffer is thoroughly mixed and incubated for 20 minutes at room temperature. Upon the completion of incubation, the assay is started by addition of 40 μL of 12.5 μM stock solution of MMP-13 fluorogenic substrate (Calbiochem Cat. No. 444235). The time-dependent increase in fluorescence is measured at the 325 nm excitation and 393 nm emission by automatic plate multireader. The IC₅₀ values are calculated from the initial reaction rates. Inhibition activity of highly potent compounds of Formula I are summarized in Table 1. Selectivity assays were run in a similar manner using MMP-1, MMP-14 and TACE.

What is claimed is:

1. A compound according to Formula (I): 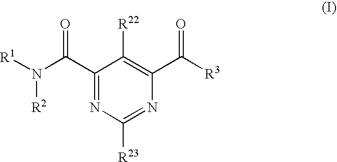
wherein:
R¹ is selected from the group consisting of alkyl, cycloalkyl-alkyl, arylalkyl, heteroarylalkyl and CHR²⁵R²¹, wherein alkyl, cycloalkyl-alkyl, arylalkyl and heteroarylalkyl are optionally substituted one or more times;
R² is hydrogen;
R³ is NR²⁰R²¹;
R¹⁰ and R¹¹ are independently selected from the group consisting of hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or R¹⁰ and R¹¹ when taken together with the nitrogen to which they are attached complete a 3- to 8-membered ring containing carbon atoms and optionally containing a heteroatom selected from O, S, or NR⁵⁰ and which is optionally substituted one or more times;
R²⁰ is selected from the group consisting of hydrogen and alkyl, wherein alkyl is optionally substituted one or more times;
R²¹is a bicyclic or tricyclic fused ring system, wherein at least one ring is partially saturated, and wherein said bicyclic or tricyclic fused ring system is optionally substituted one or more times;
R²² and R²³ are independently selected from the group consisting of hydrogen, halo, alkyl, cycloalkyl, hydroxy, alkoxy, aryl, heteroaryl, arylalkyl, heteroarylalkyl, alkenyl, alkynyl, NO₂, NR¹⁰R¹¹, NR¹⁰NR¹⁰R¹¹, NR¹⁰N═CR¹⁰R¹¹, NR¹⁰SO₂R¹¹, CN, C(O)OR¹⁰, and fluoroalkyl, wherein alkyl, cycloalkyl, alkoxy, alkenyl, alkynyl and fluoroalkyl are optionally substituted one or more times;
R²⁵ is selected from the group consisting of hydrogen, alkyl, cycloalkyl, C(O)NR¹⁰R¹¹ and haloalkyl, wherein alkyl, cycloalkyl, and haloalkyl are optionally substituted one or more times;
R⁵⁰ is selected from the group consisting of hydrogen, alkyl, aryl, heteroaryl, C(O)R⁸⁰, C(O)NR⁸⁰R⁸¹, SO₂R⁸⁰ and SO₂NR⁸⁰R⁸¹, wherein alkyl, aryl and heteroaryl are optionally substituted one or more times;
R⁸⁰ and R⁸¹ are independently selected from the group consisting of hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or R⁸⁰ and R⁸¹ when taken together with the nitrogen to which they are attached complete a 3- to 8-membered ring containing carbon atoms and optionally a heteroatom selected from O, S(O)ₓ, —NH, and —N(alkyl) and which is optionally substituted one or more times;
x is selected from 0-2; and
N-oxides, pharmaceutically acceptable salts, and stereoisomers thereof.

2. The compound according to claim 1, wherein R³ is selected from the group consisting of: 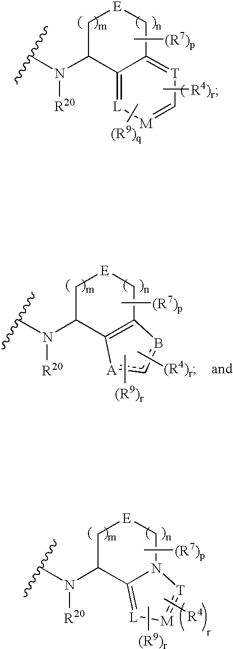
wherein:
R⁴ is selected from the group consisting of R¹⁰, hydrogen, alkyl, aryl, heteroaryl, halo, CF₃, COR¹⁰, OR¹⁰, NR¹⁰R¹¹, NO₂, CN, SO₂OR¹⁰, CO₂R¹⁰, C(O)NR¹⁰R¹¹, SO₂R¹⁰, OC(O)R¹⁰, OC(O)NR¹⁰OR¹¹, NR¹⁰C(O)R¹¹, NR¹⁰CO₂R¹¹, (C₀-C₆)-alkyl-C(═NR^{a})NHR^{b}, (C₀-C₆)-alkyl-NHC(═NR^{a})NHR^{b}, (C₀-C₆)-alkyl-C(O)OR¹⁰, (C₀-C₆)-alkyl-C(O)NR¹⁰R¹¹, (C₀-C₆)-alkyl-C(O)—NH—CN, O—(C₀-C₆)alkyl-C(O)NR¹⁰R¹¹, S(O)ₓ—(C₀-C₆-alkyl-C(O)OR¹⁰, S(O)ₓ—(C₀C₆)-alkyl-C(O)NR¹⁰R¹¹,(C₀-C₆)-alkyl-C(O)OR¹⁰,—(C₀-C₆)-alkyl-NR¹⁰R¹¹, (C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—SO₂NR¹⁰R¹¹, wherein each R⁴ group is optionally substituted by one or more R¹⁴ groups;
R⁵ is selected from the group consisting of hydrogen, alkyl, C(O)NR¹⁰R¹¹, aryl, arylalkyl, SO₂NR¹⁰R¹¹, C(O)OR¹⁰ and CN, wherein alkyl, aryl and arylalkyl are optionally substituted one or more times;
R⁷ is selected from the group consisting of hydrogen, alkyl, cycloalkyl, halo, R⁴ and NR¹⁰OR¹¹, wherein alkyl and cycloalkyl are optionally substituted one or more times;
R⁹ is selected from the group consisting of hydrogen, alkyl, CH(CH₃)CO₂H, halo, (C₀-C₆)-alkyl-C(O)OR¹⁰, (C₀-C₆)-alkyl-C(O)NR¹⁰R¹¹, (C₀-C₆)-alkyl-C(O)NH—CN, O—(C₀-C₆)-alkyl-C(O)NR¹⁰R¹¹, S(O)_{y}-alkyl-C(O)OR¹⁰, S(O)_{z}-alkyl-C(O)NR¹⁰R¹¹, (C₀-C₆)-alkyl-C(O)NR¹⁰—(C₀-C₆)-alkyl-NR¹⁰R¹¹, C(O)NR¹⁰—(C₀-C₆)-alkyl-heteroaryl, C(O)NR¹⁰—(C₀-C₆)-alkyl-aryl, CH₂NR¹⁰R¹¹, (CH₂)_{y}NR¹⁰C(O)-alkyl, (CH₂)_{w}NR¹⁰C(O)—(C₀-C₆)-alkyl-aryl, (CH₂)_{w}NR¹⁰C(O)—(C₀-C₆)-alkyl-heteroaryl, (CH₂)_{w}NR¹⁰C(O)O-alkyl, (CH₂)_{w}NR¹⁰C(O)O—(C₀-C₆)-alkyl-aryl, (CH₂)_{w}NR¹⁰C(O)O—(C₀-C₆)-alkyl-heteroaryl, (CH₂)_{w}NR¹⁰C(O)ONR¹⁰R¹¹, (CH₂)_{w}NR¹⁰S(O)₂—(C₀-C₆)-alkyl-aryl, (CH₂)_{w}NR¹⁰S(O)₂—(C₀-C₆)-alkyl-heteroaryl, (CH₂)_{w}NR¹⁰S(O)₂—NR¹⁰-alkyl, (CH₂)_{w}NR¹⁰S(O)₂NR¹⁰—(C₀-C₆)-alkyl-aryl, (CH₂)_{w}NR¹⁰S(O)₂NR¹⁰—(C₀-C₆)-alkyl-heteroaryl, (CH₂)_{w}NR¹⁰C(O)NR¹⁰—SO₂—R³⁰, S(O)₂NR¹⁰—(C₀-C₆)-alkyl-aryl, S(O)₂NR¹⁰—(C₀-C₆)-alkyl-heteoaryl, S(O)₂NR¹⁰-alkyl, S(O)₂—(C₀-C₆)-alkyl-aryl, S(O)₂—(C₀-C₆)-alkyl-heteroaryl, O-heteroaryl and heteroaryl, wherein each of said R⁹ groups is optionally substituted one or more times;
R¹⁴ is selected from the group consisting of hydrogen, alkyl, arylalkyl, cycloalkyl-alkyl, heteroarylalkyl, heterocyclylalkyl and halo, wherein alkyl, arylalkyl, cycloalkyl-alkyl, heteroarylalkyl and heterocyclylalkyl are optionally substituted one or more times;
R³⁰ is selected from the group consisting of alkyl and (C₀-C₆)-alkyl-aryl;
R^{a} and R^{b} are independently selected from the group consisting of hydrogen, CN, alkyl, haloalkyl, S(O)ₓNR¹⁰R¹¹, S(O)ₓR¹⁰ and C(O)NR¹⁰R¹¹, wherein alkyl and haloalkyl are optionally substituted one or more times;
E is selected from the group consisting of a bond, CR¹⁰R¹¹, O, NR⁵, S, S═O, S(═O)₂, C(═O), N(R¹⁰)(C═O), (C═O)N(R¹⁰), N(R¹⁰)S(═O)₂, S(═O)₂N(R¹⁰), C═N—OR¹¹, —C(R¹⁰R¹¹)C(R¹⁰R¹¹)—, —CH₂—W— and 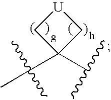
W is selected from the group consisting of O, NR⁵, S, S═O, S(═O)₂, N(R¹⁰)(C═O), N(R¹⁰)S(═O)₂ and S(═O)₂N(R¹⁰);
U is selected from the group consisting of C(⁵R¹⁰), NR⁵, O, S, S═O and S(═O)₂;
A and B are independently selected from the group consisting of C, N, O and S;
L, M and T are independently selected from the group consisting of C and N;
g and h are independently selected from 0-2;
m and n are independently selected from 0-3, provided that:
(1) when E is present, m and n are not both 3;
(2) when E is —CH₂—W—, m and n are not 3; and
(3) when E is a bond, m and n are not 0;
p is selected from 0-6;
q is selected from 0-4;
r is selected from 0-1;
w is selected from 0-4;
x is selected from 0-2;
y is selected from 1 and 2;
z is selected from 0-2; and
wherein the dotted line represents optionally a double bond.

3. The compound according to claim 2, wherein each of said R¹⁰ and R¹¹ groups is optionally substituted with one or more substituents independently selected from the group consisting of halo, CF₃, COR¹⁰, OR¹⁰, NR¹⁰R¹¹, NO₂, CN, SO₂OR¹⁰, CO₂R¹⁰, CONR¹⁰R¹¹, SO₂NR¹⁰R¹¹, SO₂R¹⁰, OC(O)R¹⁰, OC(O)NR¹⁰R¹¹, NR¹⁰C(O)R¹¹ and NR₁₀CO₂R¹¹.

4. The compound according to claim 2, wherein R²⁰ taken with the nitrogen to which it is bound and L together form a 3- to 8-membered ring containing carbon atoms and optionally containing a heteroatom selected from O, S, or NR⁵⁰ and which ring is optionally substituted.

5. The compound according to claim 2, wherein when E is present, m and n added together are 1-4.

6. The compound according to claim 2, wherein when E is present, m and n added together are 1-2.

7. The compound according to claim 2, wherein when E is a bond, m and n added together are 2-5.

8. The compound according to claim 2, wherein when E is a bond, m and n added together are 2-3.

9. The compound according to claim 2, wherein R³ is selected from the group consisting of: 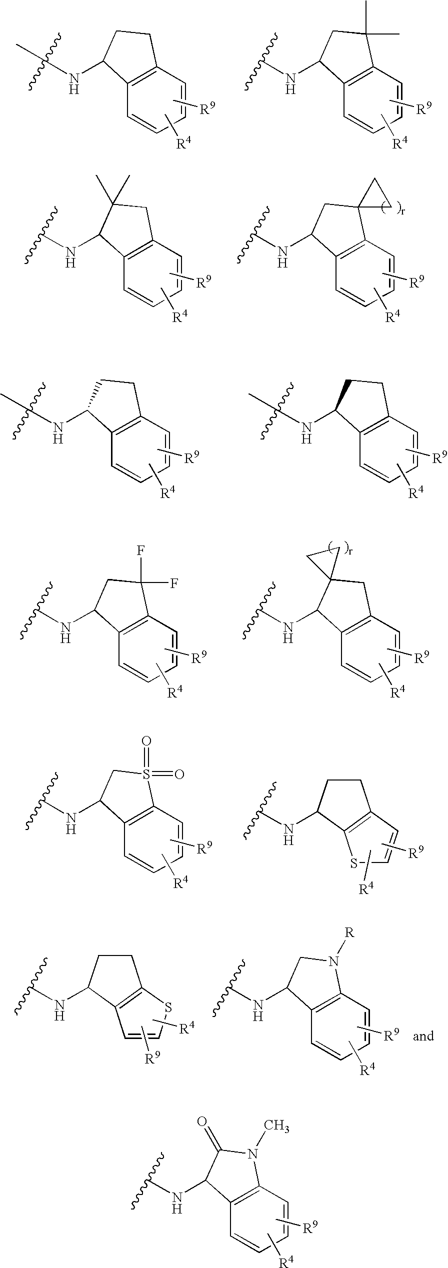
herein:
R is selected from the group consisting of C(O)NR¹⁰R¹¹, COR¹⁰, SO₂NR¹⁰R¹¹, SO₂R¹⁰, CONFCH₃ and CON(CH₃)₂, wherein C(O)NR¹⁰R¹¹, COR¹⁰, SO₂NR¹⁰R¹¹, SO₂R¹⁰, CONHCH₃ and CON(CH₃)₂ are optionally substituted one or more times;
R⁴ is selected from the group consisting of 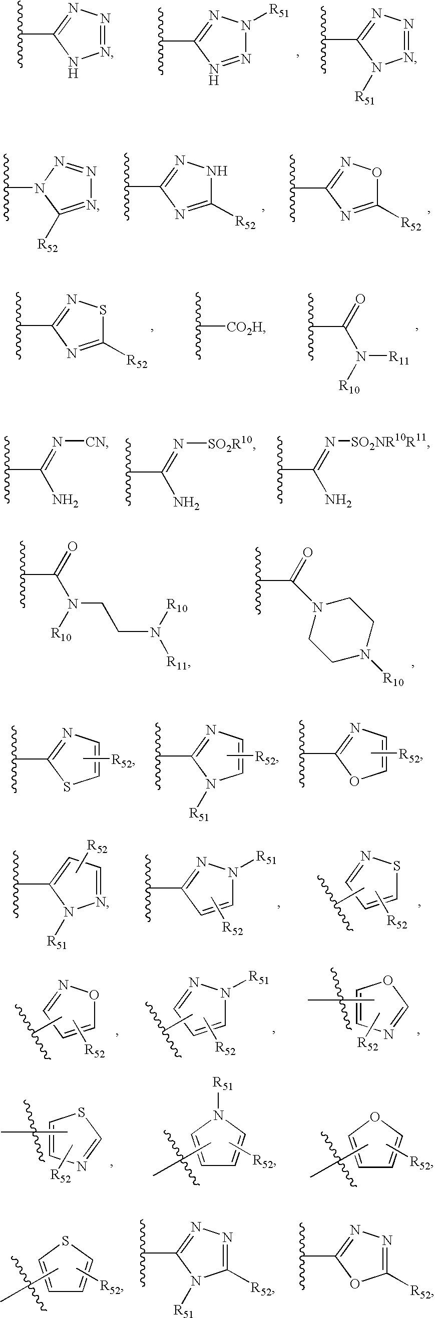 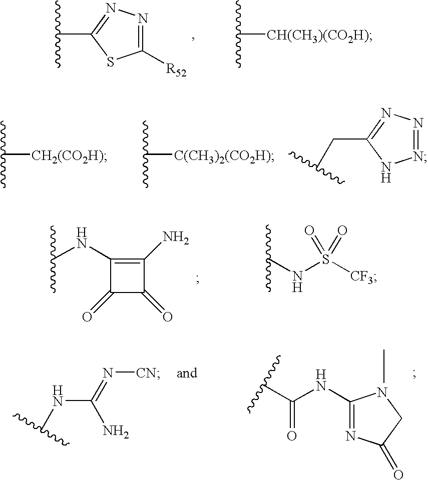
R⁵¹ is selected from the group consisting of hydrogen, alkyl, aryl, heteroaryl, arylalkyl, cycloalkylalkyl, heteroarylalkyl and haloalkyl, wherein alkyl, aryl, heteroaryl, arylalkyl, cycloalkylalkyl, heteroarylalkyl and haloalkyl are optionally substituted one or more times;
R⁵² is selected from the group consisting of hydrogen, halo, hydroxy, alkoxy, fluoroalkoxy, alkyl, aryl, heteroaryl, arylalkyl, cycloalkylalkyl, heteroarylalkyl, haloalkyl, C(O)NR¹⁰R¹¹ and O₂NR¹⁰OR¹¹, wherein alkoxy, fluoroalkoxy, alkyl, aryl, heteroaryl, arylalkyl, cycloalkylalkyl, heteroarylalkyl, haloalkyl, C(O)NR¹⁰R¹¹ and O₂NR¹⁰R¹¹ are optionally substituted one or more times; and
r is selected from 0-1.

10. The compound according to claim 2, wherein at least one R⁴ is heteroaryl.

11. The compound according to claim 10, wherein R⁴ is selected from the group consisting of dioxole, imidazole, furan, thiazole, isothiazole, isoxazole, morpholine, 1,2,4-oxadiazole, 1,3,4-oxadiazole, 1,2,4-oxadiazole, 1,2-oxazine, 1,3-oxazine, 1,4-oxazine, oxirane, oxazole, 5-oxo-1,2,4-oxadiazole, 5-oxo-1,2,4-thiadiazole, piperzine, piperidine, pyran, pyrazine, pyrazole, pyridazine, pyridine, pyrimidine, pyrrole, pyrrolidine, tetrazine, tetrazole, thiazine, 1,2,3-thiadiazole, 1,2,4-thiadiazole, 1,3,4-thiadiazole, 1,2,5-thiadiazole, thiatriazole, 1,2-thiazine, 1,3-thiazine, 1,4-thiazine, thiazole, 5-thioxo-1,2,4-diazole, thiomorpholine, thiophene, thiopyran, 1,2,3-triazine, 1,2,4-triazine, 1,3,5-triazine, 1,2,4-triazole, 1,2,3-triazole, and triazolones, which are optionally substituted.

12. The compound according to claim 1, wherein R³ is selected from the group consisting of: 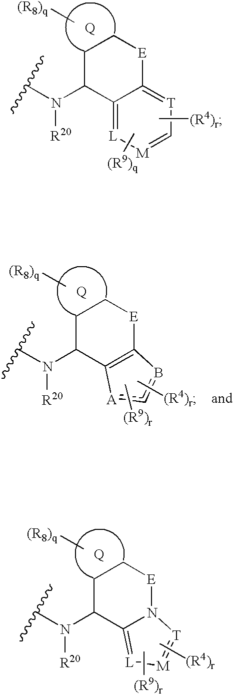
wherein:
R⁴ is selected from the group consisting of R¹⁰, hydrogen, alkyl, aryl, heteroaryl, halo, CF₃, COR¹⁰, OR¹⁰, NR¹⁰R¹¹, NO₂, CN, SO₂OR¹⁰, CO₂R¹⁰, C(O)NR¹⁰R¹¹SO₂NR¹⁰R¹¹, SO₂R¹⁰OC(O)R¹⁰, OC(O)NR¹⁰R¹¹, NR¹⁰C(O)R¹¹, NR¹⁰CO₂R¹¹, (C₀-C₆)-alkyl-C(═NR^{a})NHR^{b}, (C₀-C₆)-alkyl-NHC(═NR^{a})NHR^{b}, (C₀-C₆)-alkyl-C(O)OR¹⁰, (C₀-C₆)-alkyl-C(O)NR¹⁰R¹¹, (C₀-C₆)-alkyl-C(O)—NH—CN, O—(C₀-C₆)-alkyl-C(O)NR¹⁰R¹¹, S(O)ₓ—(C₀-C₆)-alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)—NR¹⁰R¹¹, (C₀-C₆)-alkyl-NR¹⁰C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—SO₂NR¹⁰R¹¹, wherein each R⁴ group is optionally substituted by one or more R¹⁴ groups;
R⁵ is selected from the group consisting of hydrogen, alkyl, C(O)NR¹⁰R¹¹, aryl, arylalkyl, SO₂NR¹⁰R¹¹, C(O)OR¹⁰ and CN, wherein alkyl, aryl and arylalkyl are optionally substituted one or more times;
R⁸ is selected from the group consisting of hydrogen, alkyl, OR¹⁰, NR¹⁰R¹¹, CN, arylalkyl, cycloalkyl-alkyl, heteroarylalkyl, heterocyclylalkyl and halo, wherein alkyl, arylalkyl, cycloalkyl-alkyl, heteroarylalkyl and heterocyclylalkyl are optionally substituted one or more times;
R⁹ is selected from the group consisting of hydrogen, alkyl, CH(CH₃)CO₂H, halo, (C₀-C₆)-alkyl-C(O)OR¹⁰, (C₀-C₆)-alkyl-C(O)NR¹⁰R¹¹, (C₀-C₆)-alkyl-C(O)NH—CN, O—(C₀-C₆)-alkyl-C(O)NR¹⁰R¹¹, S(O)_{y}-alkyl-C(O)OR¹⁰, S(O)_{z}-alkyl-C(O)NR¹⁰R¹¹, (C₀-C₆)-alkyl-C(O)NR¹⁰—(C₀-C₆)-alkyl-NR¹⁰R¹¹, C(O)NR¹⁰—(C₀-C₆)-alkyl-heteroaryl, C(O)NR¹⁰—(C₀-C₆)-alkyl-aryl, CH₂NR¹⁰R¹¹, (CH₂)_{y}NR¹⁰C(O)-alkyl, (CH₂)_{w}NR¹⁰C(O)—(C₀-C₆)-alkyl-aryl, (CH₂)_{w}NR¹⁰C(O)—(C₀-C₆)-alkyl-heteroaryl, (CH₂)_{w}NR¹⁰C(O)O-alkyl, (CH₂),NR¹⁰C(O)O—(C₀-C₆)-alkyl-aryl, (CH₂)_{w}NR¹⁰C(O)O—(C₀-C₆)-alkyl-heteroaryl, (CH₂)_{w}NR¹⁰C(O)ONR¹⁰R¹¹, (CH₂)_{w}NR¹⁰S(O)₂—(C₀-C₆)-alkyl-aryl, (CH₂)_{w}NR¹⁰S(O)₂—(C₀-C₆)-alkyl-heteroaryl, (CH₂)_{w}NR¹⁰S(O)₂—NR¹⁰-alkyl, (CH₂)_{w}NR¹⁰S(O)₂NR¹⁰—(C₀-C₆)-alkyl-aryl, (CH₂)_{w}NR¹⁰S(O)₂NR¹⁰—(C₀-C₆)-alkyl-heteroaryl, (CH₂)_{w}NR¹⁰C(O)NR¹⁰—SO₂—R³⁰, S(O)₂NR¹⁰—(C₀-C₆)-alkyl-aryl, S(O)₂NR¹⁰—(C₀-C₆)-alkyl-heteroaryl, S(O)₂NR¹⁰-alkyl, S(O)₂—(C₀-C₆)-alkyl-aryl, S(O)₂—(C₀-C₆)-alkyl-heteroaryl, O-heteroaryl and heteroaryl, wherein each of said R⁹ groups is optionally substituted one or more times;
R³⁰ is selected from the group consisting of alkyl and (C₀-C₆)-alkyl-aryl;
R^{a} and R^{b} are independently selected from the group consisting of hydrogen, CN, alkyl, haloalkyl, S(O)ₓNR¹⁰R¹¹, S(O)ₓR¹⁰ and C(O)NR¹⁰R¹¹, wherein alkyl and haloalkyl are optionally substituted one or more times;
E is selected from the group consisting of a bond, CR¹⁰R¹¹, O, NR⁵, S, S═O, S(═O)₂, C(═O), N(R¹⁰)(C═O), (C═O)N(R¹⁰), N(R¹⁰)S(═O)₂, S(═O)₂N(R¹⁰), C═N—OR¹¹, —C(R¹⁰R¹¹)C(R¹⁰R¹¹)—, —CH₂—W— and 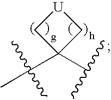
W is selected from the group consisting of O, NR⁵, S, S═O, S(═O)₂, N(R¹⁰)(C═O), N(R¹⁰)S(═O)₂ and S(═O)₂N(R¹⁰);
U is selected from the group consisting of C(R⁵R¹⁰), NR⁵, O, S, S═O and S(═O)₂;
Q is selected from the group consisting of 3-7 membered cycloalkyl, 4-7 membered heterocyclyl, 5-6 membered heteroaryl and 6-membered aryl;
A and B are independently selected from the group consisting of C, N, O and S;
L, M and T are independently selected from the group consisting of C and N;
g and h are independently selected from 0-2;
q is selected from 0-4;
r is selected from 0-1;
w is selected from 0-4;
x is selected from 0-2;
y is selected from 1 and 2;
z is selected from 0-2; and
wherein the dotted line represents optionally a double bond.

13. The compound according to claim 12, wherein R³ comprises:

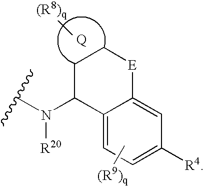

14. The compound according to claim 13, wherein R⁴ is selected from the group consisting of dioxole, imidazole, furan, thiazole, isothiazole, isoxazole, morpholine, 1,2,4-oxadiazole, 1,3,4-oxadiazole, 1,2,4-oxadiazole, 1,2-oxazine, 1,3-oxazine, 1,4-oxazine, oxirane, oxazole, 5-oxo-1,2,4-oxadiazole, 5-oxo-1,2,4-thiadiazole, piperzine, piperidine, pyran, pyrazine, pyrazole, pyridazine, pyridine, pyrimidine, pyrrole, pyrrolidine, tetrazine, tetrazole, thiazine, 1,2,3-thiadiazole, 1,2,4-thiadiazole, 1,3,4-thiadiazole, 1,2,5-thiadiazole, thiatriazole, 1,2-thiazine, 1,3-thiazine, 1,4-thiazine, thiazole, 5-thioxo-1,2,4-diazole, thiomorpholine, thiophene, thiopyran, 1,2,3-triazine, 1,2,4-triazine, 1,3,5-triazine, 1,2,4-triazole, 1,2,3-triazole, and triazolones, which are optionally substituted.

15. The compound according to claim 1, wherein R¹ is selected from the group consisting of: 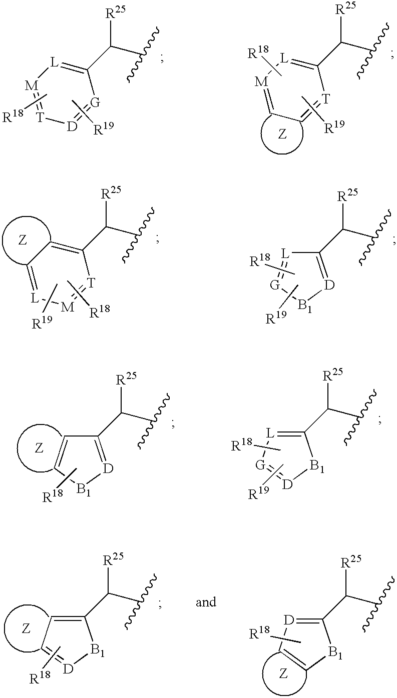
wherein:
R¹⁸ and R¹⁹ are independently selected from the group consisting of hydrogen, alkyl, haloalkyl, alkynyl, OH, halo, CN, C(O)NR¹⁰R¹¹, CO₂R¹⁰, OR¹⁰, OCF₃, OCHF₂, NR¹⁰CONR¹⁰R¹¹,NR¹⁰COR¹¹,NR¹⁰SO₂R¹¹,NR¹⁰SO₂NR¹⁰R¹¹, SO₂NR¹⁰R¹¹ and NR¹⁰R¹¹, wherein alkyl, alkynyl and haloalkyl are optionally substituted one or more times;
R²⁵ is selected from the group consisting of hydrogen, alkyl, cycloalkyl, C(O)NR¹⁰R¹¹ and haloalkyl, wherein alkyl, cycloalkyl, and haloalkyl are optionally substituted one or more times;
B₁ is selected from the group consisting of NR¹⁰, O and S;
D, G, L, M and T are independently selected from the group consisting of C and N; and
Z is a 5- to 6-membered ring selected from the group consisting of cycloalkyl, heterocycloalkyl, aryl and heteroaryl, wherein cycloalkyl, heterocycloalkyl, aryl and heteroaryl are optionally substituted one or more times.

16. The compound according to claim 15, wherein R¹ is selected from the group consisting of:

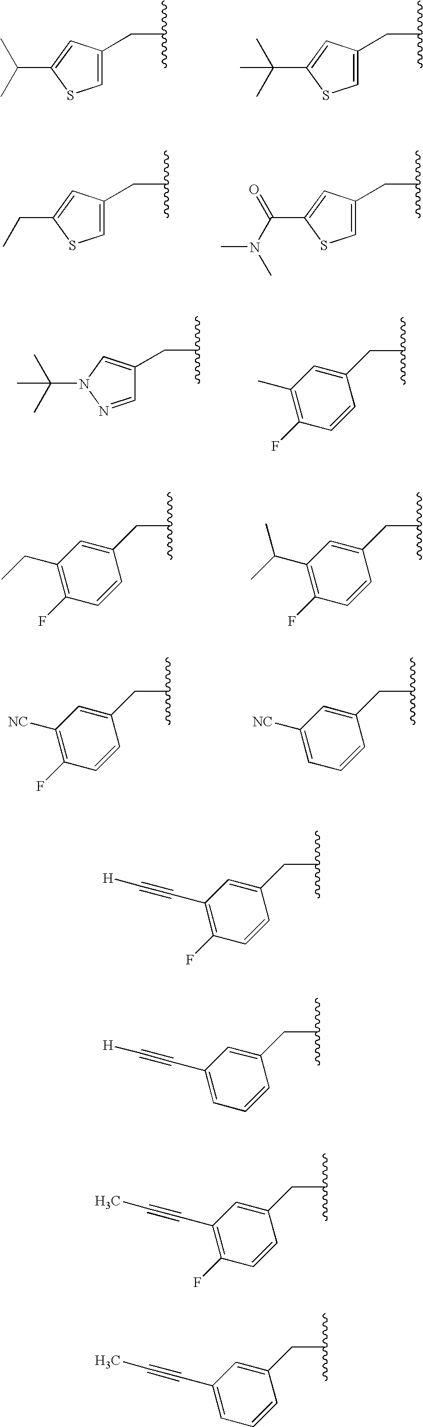

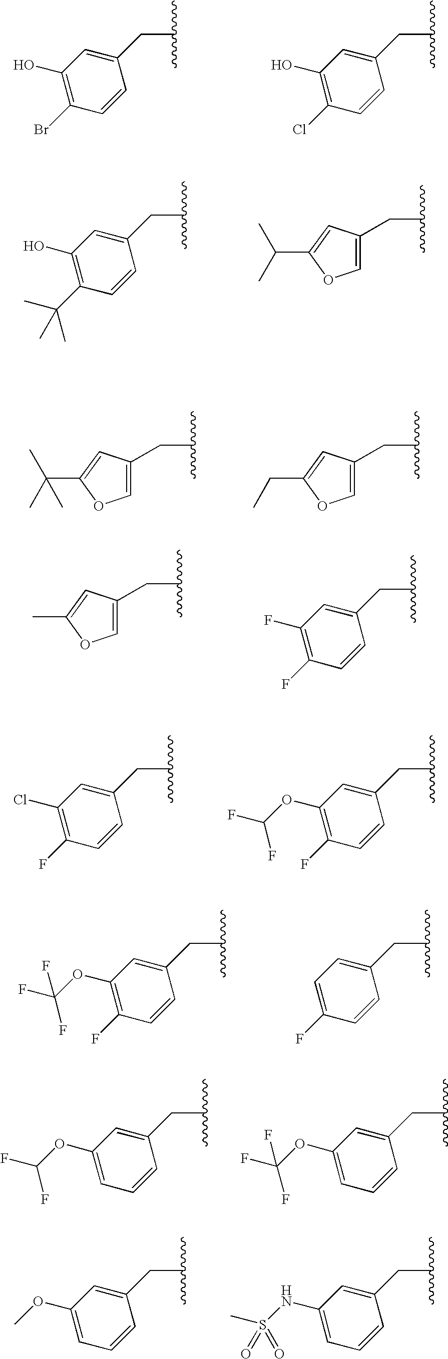

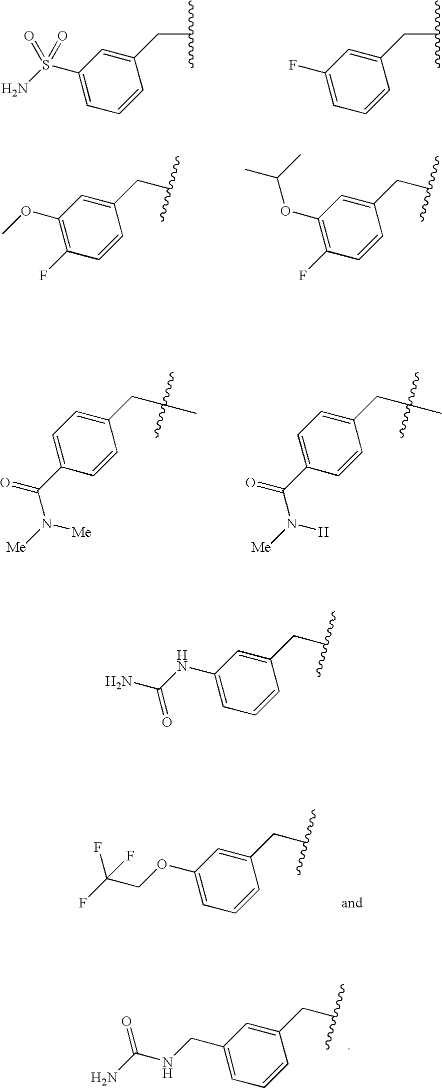

17. The compound according to claim 1, wherein R¹ is selected from the group consisting of: 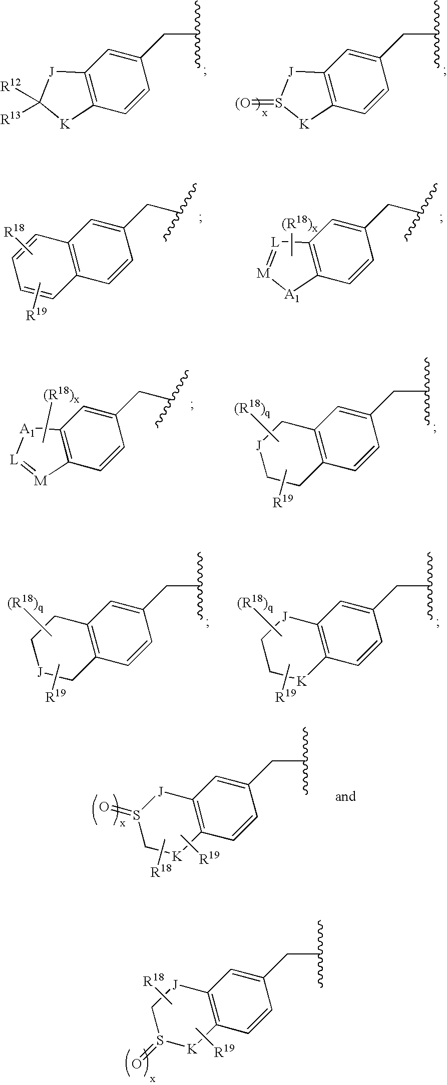
wherein:
R¹² and R¹³ are independently selected from the group consisting of hydrogen, alkyl and halo, wherein alkyl is optionally substituted one or more times, or optionally R¹² and R¹³ together form ═O, ═S or ═NR¹⁰;
R¹⁸ and R¹⁹ are independently selected from the group consisting of hydrogen, alkyl, haloalkyl, alkynyl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, alkynyl and haloalkyl are optionally substituted one or more times, or optionally two R¹⁸ groups together form ═O, ═S or ═NR¹⁰;
J and K are independently selected from the group consisting of CR¹⁰R¹¹, NR¹⁰, O and S(O)ₓ;
A₁ is selected from the group consisting of NR¹⁰, O and S;
L and M are independently selected from the group consisting of C and N;
q is selected from 0-4; and
x is selected from 0-2.

18. The compound according to claim 17, wherein R¹ is selected from the group consisting of:

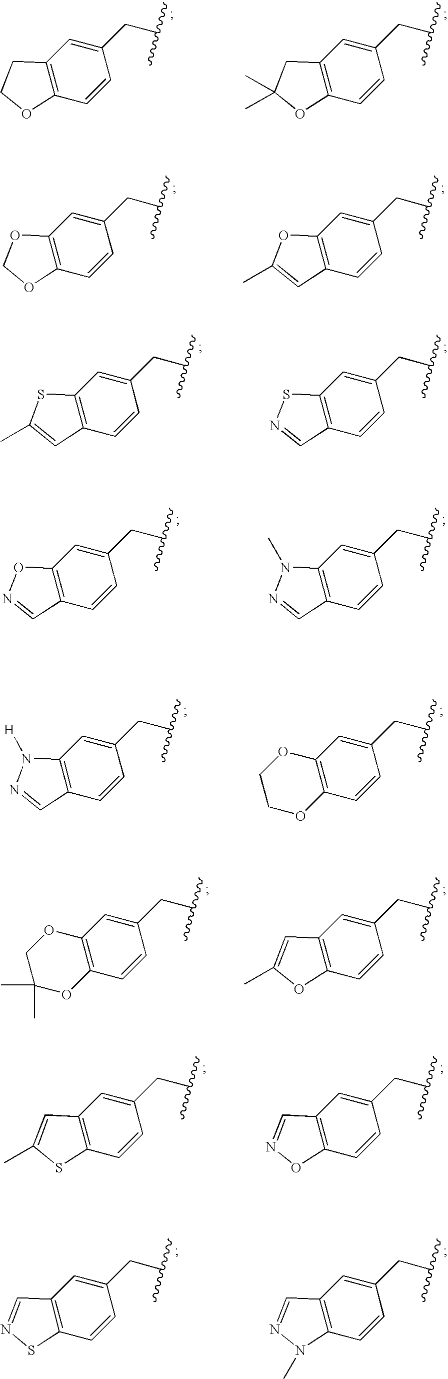

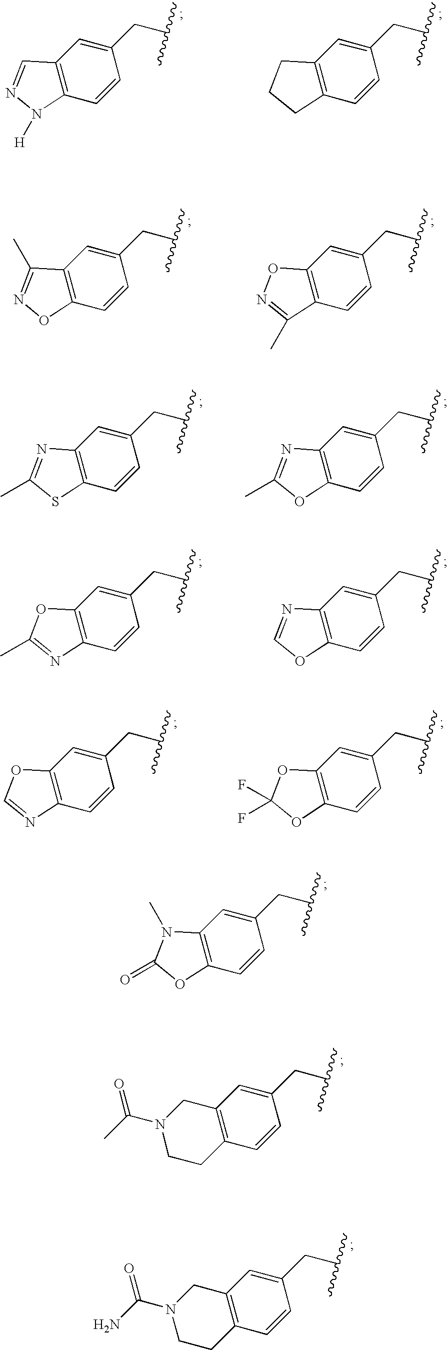

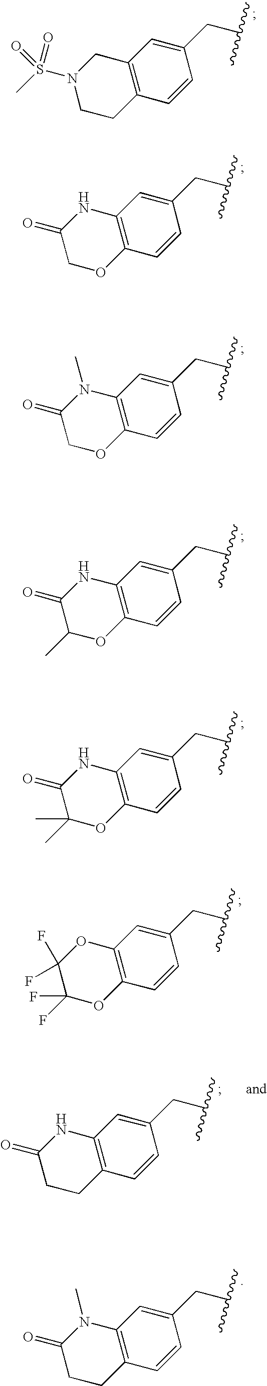

19. The compound according to claim 1, wherein R¹ is selected from the group consisting of: 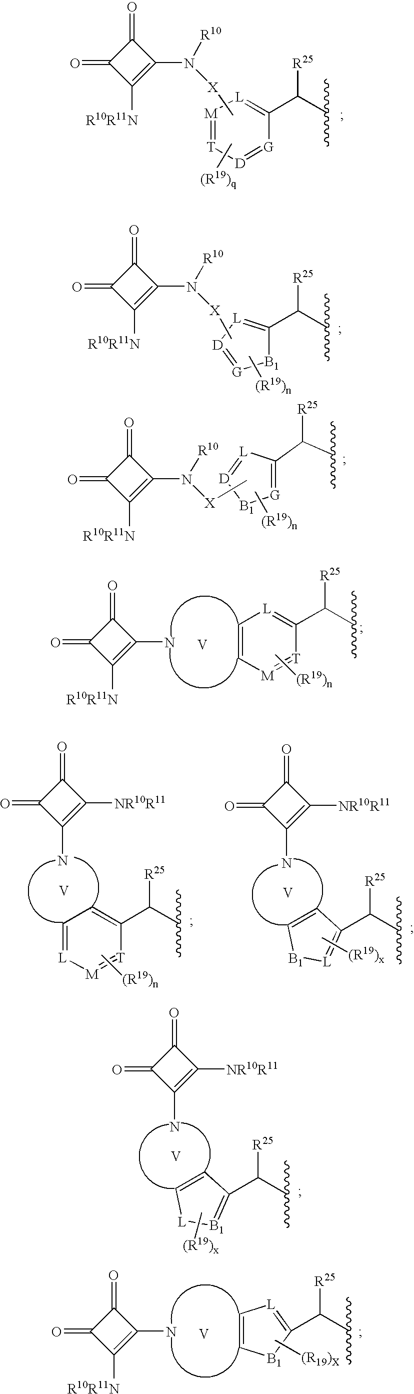 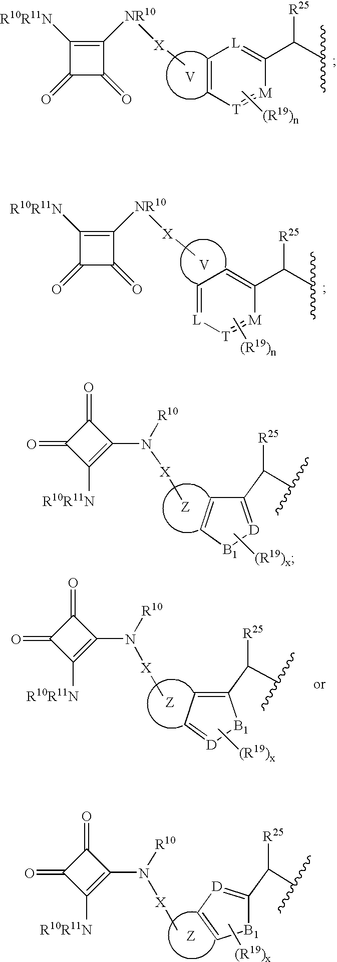
wherein:
R⁵ is selected from the group consisting of hydrogen, alkyl, C(O)NR¹⁰R¹¹, aryl, arylalkyl, SO₂NR¹⁰R¹¹, C(O)OR¹⁰ and CN, wherein alkyl, aryl and arylalkyl are optionally substituted one or more times;
R¹⁹ is selected from the group consisting of hydrogen, alkyl, haloalkyl, alkynyl, OH, halo, CN, C(O)NR¹⁰R¹¹, CO₂R¹⁰, OR¹⁰, OCF₃, OCHF₂, NR¹⁰ CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, alkynyl and haloalkyl are optionally substituted one or more times;
R²⁵ is selected from the group consisting of hydrogen, alkyl, cycloalkyl, C(O)NR¹⁰R¹¹ and haloalkyl, wherein alkyl, cycloalkyl, and haloalkyl are optionally substituted one or more times;
D, G, L, M and T are independently selected from the group consisting of C and N;
B, is selected from the group consisting of NR¹⁰, O and S;
X is selected from the group consisting of a bond and (CR¹⁰R¹¹)_{w}E(CR¹⁰R¹¹)_{w};
E is selected from the group consisting of a bond, CR¹⁰R¹¹, O, NR⁵, S, S═O, S(═O )₂, C(═O), N(R¹⁰)(C═O), (C═O)N(R¹⁰), N(R¹⁰)S(═O )₂, S(═O)₂N(R¹⁰), C═N—OR¹¹, —C(R¹⁰R¹¹)C(R¹⁰R¹¹)—, —CH₂—W— and 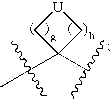
W is selected from the group consisting of O, NR⁵, S, S═O, S(═O)₂, N(R¹⁰)(C═O), N(R¹⁰)S(═O)₂ and S(═O)₂N(R¹⁰);
U is selected from the group consisting of C(R⁵R¹⁰), NR⁵, O, S, S═O and S(═O)₂;
n is selected from 0 to 3;
q is selected from 0-4;
w is selected of 0-4;
x is selected from 0-2;
V is a 5- to 8-membered ring selected from the group consisting of cycloalkyl, heterocycloalkyl, aryl and heteroaryl, which is optionally substituted one or more times; and
Z is a 5- to 6-membered ring selected from the group consisting of cycloalkyl, heterocycloalkyl, aryl, and heteroaryl, wherein cycloalkyl, heterocycloalkyl, aryl, and heteroaryl are optionally substituted one or more times.

20. The compound of claim 19, wherein R¹ is selected from the group consisting of: 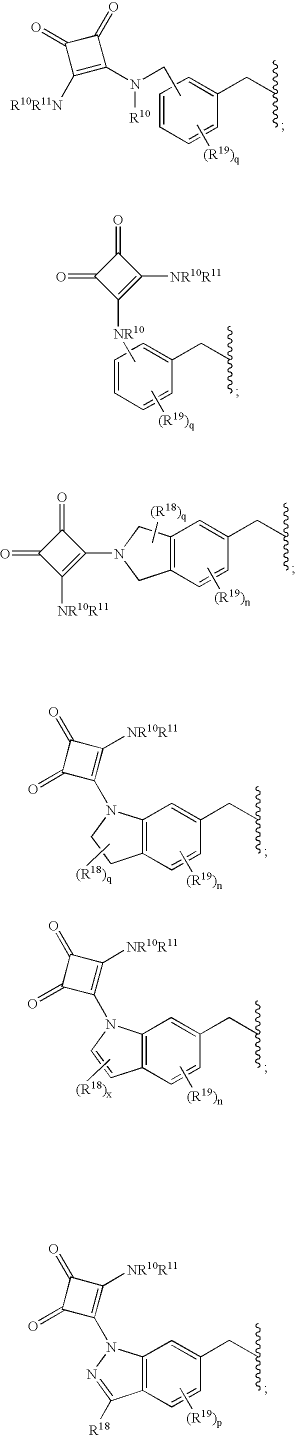 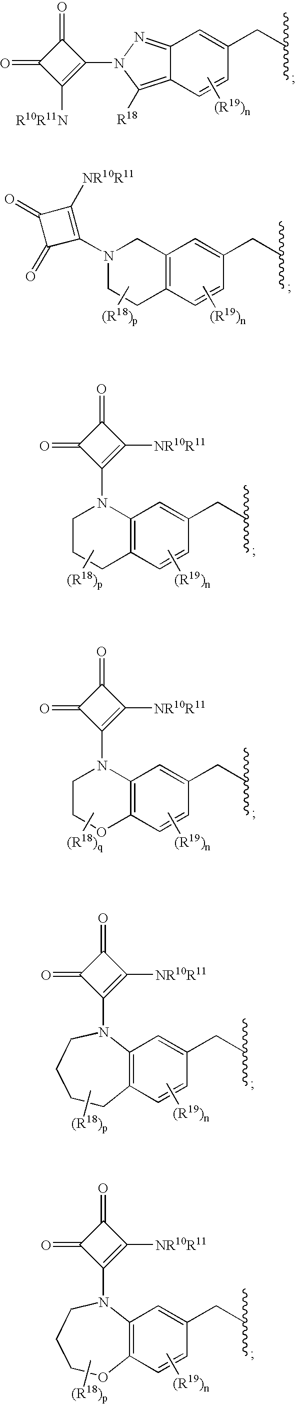 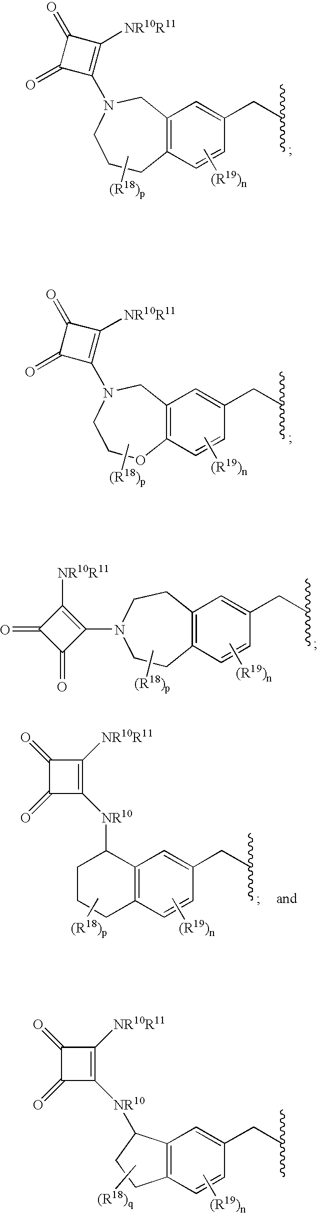
wherein:
R¹⁸ and R¹⁹ are independently selected from the group consisting of hydrogen, alkyl, haloalkyl, alkynyl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, alkynyl and haloalkyl are optionally substituted one or more times, or optionally two R¹⁸ groups together form ═O, ═S or ═NR¹⁰; and
p is selected from 0-6.

21. The compound of claim 20, wherein R¹ is selected from the group consisting of:

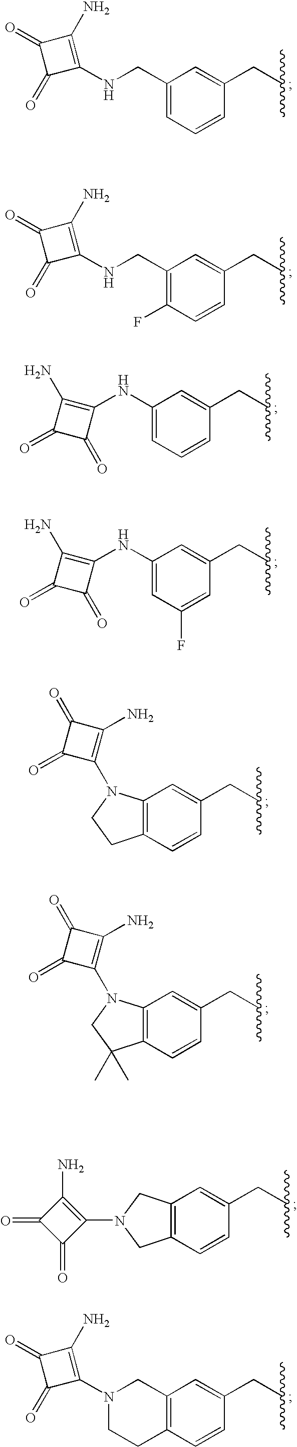

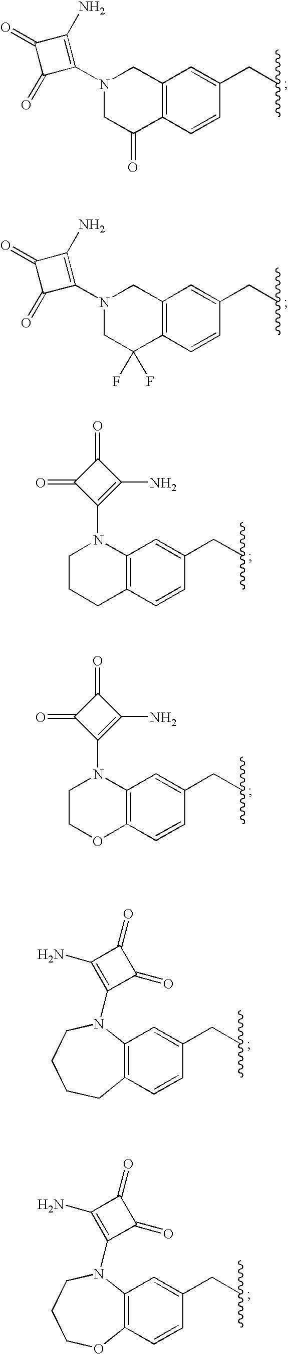

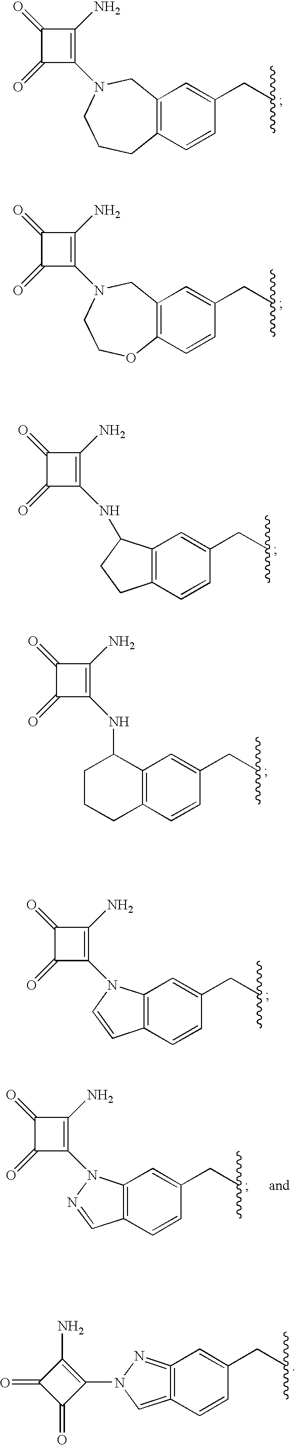

22. The compound according to claim 1, which is a compound of Formula (II): 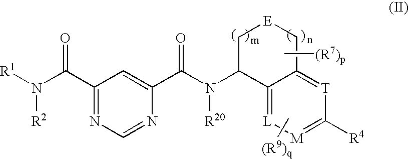
wherein:
R⁴ is selected from the group consisting of R¹⁰, hydrogen, alkyl, aryl, heteroaryl, halo, CF₃, COR¹⁰, OR¹⁰O, NR¹⁰R¹¹, NO₂, CN, SO₂OR¹⁰, CO₂R¹⁰, C(O)NR¹⁰R¹¹, SO₂NR¹⁰R¹¹, SO₂R¹⁰, OC(O)R¹⁰, OC(O)NR¹⁰R¹¹, NR¹⁰C(O)R¹¹, NR¹⁰CO₂R¹¹, (C₀-C₆)-alkyl-C(═NR^{a})NHR^{b}, (C₀-C₆)-alkyl-NHC(═NR^{a})NHR^{b}, (C₀-C₆)-alkyl-C(O)OR¹⁰, (C₀-C₆)-alkyl-C(O)NR¹⁰R¹¹, (C₀-C₆)-alkyl-C(O)—NH—CN, O—(C₀-C₆)-alkyl-C(O)NR¹⁰R¹¹, S(O)ₓ—(C₀-C₆)-alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰-SO₂NR¹⁰R¹¹, wherein each R⁴ group is optionally substituted by one or more R¹⁴ groups;
R⁵ is selected from the group consisting of hydrogen, alkyl, C(O)NR¹⁰R¹¹, aryl, arylalkyl, SO₂NR¹⁰R¹¹, C(O)OR¹⁰ and CN, wherein alkyl, aryl and arylalkyl are optionally substituted one or more times;
R⁷ is selected from the group consisting of hydrogen, alkyl, cycloalkyl, halo, R⁴ and NROR ¹⁰R¹¹, wherein alkyl and cycloalkyl are optionally substituted one or more times;
R⁹ is selected from the group consisting of hydrogen, alkyl, CH(CH₃)CO₂H, halo, (C₀-C₆)-alkyl-C(O)OR¹⁰, (C₀-C₆)-alkyl-C(O)NR¹⁰R¹¹, (C₀-C₆)-alkyl-C(O)NH—CN, O—(C₀-C₆)-alkyl-C(O)NR¹⁰R¹¹, S(O)_{y}-alkyl-C(O)OR¹⁰, S(O)_{z}-alkyl-C(O)NR¹⁰R¹¹, (C₀-C₆)-alkyl-C(O)NR¹⁰—(C₀-C_{C}₆)-alkyl-NR¹⁰R¹¹, C(O)NR¹⁰—(C₀-C₆)-alkyl-heteroaryl, C(O)NR¹⁰—)C₀-C₆)-alkyl-aryl, CH₂NR¹⁰R¹¹, (CH₂)_{y}NR¹⁰C(O)-alkyl, (CH₂)_{w}NR¹⁰C(O)—(C₀-C₆)-alkyl-aryl, (CH₂),NR¹⁰C(O)—(C₀-C₆)-alkyl-heteroaryl, (CH₂)_{w}NR¹⁰C(O)O-alkyl, (CH₂)_{w}NR¹⁰C(O)O—(C₀-C₆)-alkyl-aryl, (CH₂)_{w}NR¹⁰C(O)O—(C₀-C₆)-alkyl-heteroaryl, (CH₂)_{w}NR¹⁰C(O)ONR¹⁰R¹¹, (CH₂)_{w}NR¹⁰S(O)₂—(C₀-C₆)-alkyl-aryl, (CH₂)_{w}NR¹⁰S(O)₂—(C₀-C₆)-alkyl-heteroaryl, (CH₂)_{w}NR¹⁰S(O)₂—NR¹⁰-alkyl, (CH₂)_{w}NR¹⁰S(O)₂NR¹⁰—(C₀-C₆)-alkyl-aryl, (CH₂)_{w}NR¹⁰S(O)₂NR¹⁰—(C₀-C₆)-alkyl-heteroaryl, (CH₂)_{w}NR¹⁰C(O)NR¹⁰—SO₂—R³⁰, S(O)₂NR¹⁰—(C₀-C₆)-alkyl-aryl, S(O)₂NR¹⁰—(C₀-C₆)-alkyl-heteroaryl, S(O)₂NR¹⁰-alkyl, S(O)₂—(C₀-C₆)-alkyl-aryl, S(O)₂—(C₀-C₆)-alkyl-heteroaryl, O- heteroaryl and heteroaryl, wherein each of said R⁹ groups is optionally substituted one or more times;
R¹⁴ is selected from the group consisting of hydrogen, alkyl, arylalkyl, cycloalkyl-alkyl, heteroarylalkyl, heterocyclylalkyl and halo, wherein alkyl, arylalkyl, cycloalkyl-alkyl, heteroarylalkyl and heterocyclylalkyl are optionally substituted one or more times;
R³⁰ is selected from the group consisting of alkyl and (C₀-C₆)-alkyl-aryl;
R^{a} and R^{b} are independently selected from the group consisting of hydrogen, CN, alkyl, haloalkyl, S(O)ₓNR¹⁰R¹¹, S(O)ₓR¹⁰ and C(O)NR¹⁰R¹¹, wherein alkyl and haloalkyl are optionally substituted one or more times;
E is selected from the group consisting of a bond, CR¹⁰R¹¹, O, NR⁵, S, S═O, S(═O)₂, C(═O), N(R¹⁰)(C═O), (C═O)N(R¹⁰), N(R¹⁰)S(═O)₂, S(═O )₂N(R¹⁰), C═N—OR¹¹, —C(R¹⁰R¹¹)C(R¹⁰R¹¹)—, —CH₂—W— and 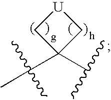
W is selected from the group consisting of O, NR⁵, S, S═O, S(═O)₂, N(R¹⁰)(C═O), N(R¹⁰)S(═O )₂ and S(═O )₂N(R¹⁰);
U is selected from the group consisting of C(R⁵R¹⁰), NR⁵, O, S, S═O and S(═O )₂;
L, M and T are independently selected from the group consisting of C and N;
g and h are independently selected from 0-2;
m and n are independently selected from 0-3, provided that:
(1) when E is present, m and n are not both 3;
(2) when E is —CH₂—W—, m and n are not 3; and
(3) when E is a bond, m and n are not 0;
p is selected from 0-6;
q is selected from 0-4;
w is selected from 0-4;
x is selected from 0-2;
y is selected from 1 and 2; and
z is selected from 0-2.

23. The compound according to claim 1, which is a compound of Formula (III): 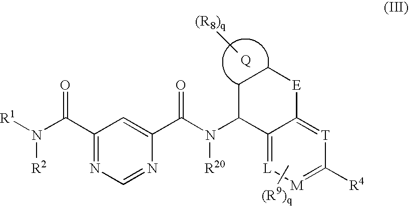
wherein:
R⁴ is selected from the group consisting of R¹⁰, hydrogen, alkyl, aryl, heteroaryl, halo, CF₃, COR¹⁰, OR¹⁰, NR¹⁰R¹¹, NO₂, CN, SO₂OR¹⁰, CO₂R¹⁰, C(O)NR¹⁰R¹¹, SO₂NR¹⁰R¹¹, SO₂R¹⁰, OC(O)R¹⁰, OC(O)NR¹⁰R¹¹, NR¹⁰C(O)R¹¹, NR¹⁰CO₂R¹¹, (C₀-C₆)-alkyl-C(═NR^{a})NHR^{b}, (C₀-C₆)-alkyl-NHC(═NR^{a})NHR^{b}, (C₀-C₆)-alkyl-C(O)OR¹⁰, (C₀-C₆)-alkyl-C(O)NR¹⁰R¹¹, (C₀-C₆)-alkyl-C(O)—NH—CN, O—(C₀-C₆)-alkyl-C(O)NR¹⁰R¹¹, S(O)ₓ—(C₀-C₆)-alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—SO₂NR¹⁰R¹¹, wherein each R⁴ group is optionally substituted by one or more R¹⁴ groups;
R⁵ is selected from the group consisting of hydrogen, alkyl, C(O)NR¹⁰R¹¹, aryl, arylalkyl, SO₂NR¹⁰R¹¹, C(O)OR¹⁰ and CN, wherein alkyl, aryl and arylalkyl are optionally substituted one or more times;
R⁸ is selected from the group consisting of hydrogen, alkyl, OR¹⁰, NR¹⁰R¹¹, CN, arylalkyl, cycloalkyl-alkyl, heteroarylalkyl, heterocyclylalkyl and halo, wherein alkyl, arylalkyl, cycloalkyl-alkyl, heteroarylalkyl and heterocyclylalkyl are optionally substituted one or more times;
R⁹ is selected from the group consisting of hydrogen, alkyl, CH(CH₃)CO₂H, halo, (C₀-C₆)-alkyl-C(O)OR¹⁰, (C₀-C₆)-alkyl-C(O)NR¹⁰R¹¹, (C₀-C₆)-alkyl-C(O)NH—CN, O—(C₀-C₆)-alkyl-C(O)NR¹⁰R¹¹, S(O)_{y}-alkyl-C(O)OR¹⁰, S(O)_{z}-alkyl-C(O)NR¹⁰R¹¹, (C₀-C₆)-alkyl-C(O)NR¹⁰—(C₀-C₆)-alkyl-NR¹⁰R¹¹, C(O)NR¹⁰—(C₀-C₆)-alkyl-heteroaryl, C(O)NR¹⁰—(C₀-C₆)-alkyl-aryl, CH₂NR¹⁰R¹¹, (CH₂)_{y}NR¹⁰C(O)-alkyl, (CH₂)_{w}NR¹⁰C(O)—(C₀-C₆)-alkyl-aryl, (CH₂)_{w}NR¹⁰C(O)—(C₀-C₆)-alkyl-heteroaryl, (CH₂)_{w}NR¹⁰C(O)O-alkyl, (CH₂)_{w}NR¹⁰C(O)O—(C₀-C₆)-alkyl-aryl, (CH₂)_{w}NR¹⁰C(O)O—(C₀-C₆)-alkyl-heteroaryl, (CH₂)_{w}NR¹⁰C(O)ONR¹⁰R¹¹, (CH₂),NR¹⁰S(O)₂—(C₀-C₆)-alkyl-aryl, (CH₂)_{w}NR¹⁰S(O)₂—(C₀-C₆)-alkyl-heteroaryl, (CH₂)_{w}NR¹⁰S(O)₂—NR¹⁰O-alkyl, (CH₂)_{w}NR¹⁰S(O)₂NR¹⁰—(C₀-C₆)-alkyl-aryl, (CH₂)_{w}NR¹⁰S(O)₂NR¹⁰—(C₀-C₆)-alkyl-heteroaryl, (CH₂)_{w}NR¹⁰C(O)NR¹⁰—SO₂—R³⁰, S(O)₂NR¹⁰—(C₀-C₆)-alkyl-aryl, S(O)₂NR¹⁰—(C₀-C₆)-alkyl-heteroaryl, S(O)₂NR¹⁰-alkyl, S(O)₂—(C₀-C₆)-alkyl-aryl, S(O)₂—(C₀-C₆)-alkyl-heteroaryl, O-heteroaryl and heteroaryl, wherein each of said R⁹ groups is optionally substituted one or more times;
R¹⁴ is selected from the group consisting of hydrogen, alkyl, arylalkyl, cycloalkyl-alkyl, heteroarylalkyl, heterocyclylalkyl and halo, wherein alkyl, arylalkyl, cycloalkyl-alkyl, heteroarylalkyl and heterocyclylalkyl are optionally substituted one or more times;
R³⁰ is selected from the group consisting of alkyl and (C₀-C₆)-alkyl-aryl;
Ra^{a} and R^{b} are independently selected from the group consisting of hydrogen, CN, alkyl, haloalkyl, S(O)ₓNR¹⁰R¹¹, S(O)ₓR¹⁰ and C(O)NR¹⁰R¹¹, wherein alkyl and haloalkyl are optionally substituted one or more times;
E is selected from the group consisting of a bond, CR¹⁰R¹¹, O, NR⁵, S, S═O, S(═O)₂, C(═O), N(R¹⁰)(C═O), (C═O)N(R¹⁰), N(R¹⁰)S(═O)₂, S(═O)₂N(R¹⁰), C═N—OR¹¹, —C(R¹⁰R¹¹)C(R¹⁰R¹¹)—,—CH₂—W— and 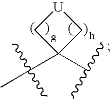
W is selected from the group consisting of O, NR⁵, S, S═O, S(═O)₂, N(R¹⁰)(C═O), N(R¹⁰)S(═O)₂ and S(═O)₂N(R¹⁰);
U is selected from the group consisting of C(R⁵R¹⁰), NR⁵, O, S, S═O and S(═O)₂;
Q is selected from the group consisting of 3-7 membered cycloalkyl, 4-7 membered heterocyclyl, 5-6 membered heteroaryl and 6-membered aryl;
L, M and T are independently selected from the group consisting of C and N;
q is selected from 0-4;
w is selected from 0-4;
x is selected from 0-2;
y is selected from 1 and 2; and
z is selected from 0-2.

24. A compound according to Formula (IV): 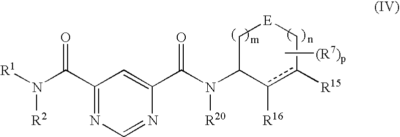
wherein:
R¹ is selected from the group consisting of alkyl, cycloalkyl-alkyl, arylalkyl, heteroarylalkyl and CHR²⁵R²¹, wherein alkyl, cycloalkyl-alkyl, arylalkyl and heteroarylalkyl are optionally substituted one or more times;
R² is hydrogen;
R⁴ is selected from the group consisting of R¹⁰, hydrogen, alkyl, aryl, heteroaryl, halo, CF₃, COR¹⁰, OR¹⁰, NR¹⁰R¹¹, NO₂, CN, SO₂OR¹⁰, CO₂R¹⁰, C(O)NR¹⁰R¹¹, SO₂NR¹¹, SO₂R¹⁰, OC(O)R¹⁰, OC(O)NR¹⁰R¹¹, NR¹⁰C(O)R¹¹, NR¹⁰CO₂R¹¹, (C₀-C₆)-alkyl-C(═NR^{a})NHR^{b}, (C₀-C₆)-alkyl-NHC)═NR^{a})NHR^{b}, (C₀-C₆)-alkyl-C(O)OR¹⁰, (C₀-C₆)-alkyl-C(O)NR¹⁰R¹¹, (C₀-C₆)-alkyl-C(O)—NH—CN, O—(C₀-C₆)-alkyl-C(O)NR¹⁰R¹¹, S(O)ₓ—(C₀-C₆)-alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—SO₂NR¹⁰R¹¹, wherein each R group is optionally substituted by one or more R¹⁴ groups;
R⁵ is selected from the group consisting of hydrogen, alkyl, C(O)NR¹⁰R¹¹, aryl, arylalkyl, SO₂NR¹⁰R¹¹, C(O)OR¹⁰ and CN, wherein alkyl, aryl and arylalkyl are optionally substituted one or more times;
R⁷ is selected from the group consisting of hydrogen, alkyl, cycloalkyl, halo, R⁴ and NR¹⁰R¹¹, wherein alkyl and cycloalkyl are optionally substituted one or more times;
R¹⁰ and R¹¹ are independently selected from the group consisting of hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or R¹⁰ and R¹¹ when taken together with the nitrogen to which they are attached complete a 3- to 8-membered ring containing carbon atoms and optionally containing a heteroatom selected from O, S, or NR⁵⁰ and which is optionally substituted one or more times;
R¹⁴ is selected from the group consisting of hydrogen, alkyl, arylalkyl, cycloalkyl-alkyl, heteroarylalkyl, heterocyclylalkyl and halo, wherein alkyl, arylalkyl, cycloalkyl-alkyl, heteroarylalkyl and heterocyclylalkyl are optionally substituted one or more times;
R¹⁵ and R¹⁶ when taken together with the carbon atoms to which they are bound, form a ring selected from the group consisting of 6-membered aryl ring, 5- or 6-membered heteroaryl ring, 5- to 8-membered cycloalkyl ring, 5- to 8-membered heterocyclyl ring, 5- to 8-membered cycloalkenyl ring and 5- to 8-membered heterocycloalkenyl ring, wherein said ring is optionally substituted by one or more R⁴ groups;
R²⁰ is selected from the group consisting of hydrogen and alkyl, wherein alkyl is optionally substituted one or more times;
R²¹ is a bicyclic or tricyclic fused ring system, wherein at least one ring is partially saturated, and wherein said bicyclic or tricyclic fused ring system is optionally substituted one or more times;
R²⁵ is selected from the group consisting of hydrogen, alkyl, cycloalkyl, C(O)NR¹⁰R¹¹ and haloalkyl, wherein alkyl, cycloalkyl, and haloalkyl are optionally substituted one or more times;
R⁵⁰ is selected from the group consisting of hydrogen, alkyl, aryl, heteroaryl, C(O)R⁸⁰, C(O)NR⁸⁰R⁸¹, SO₂R⁸⁰ and SO₂NR⁸⁰R⁸¹, wherein alkyl, aryl and heteroaryl are optionally substituted one or more times;
R⁸⁰ and R⁸¹ are independently selected from the group consisting of hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or R⁸⁰ and R⁸¹ when taken together with the nitrogen to which they are attached complete a 3- to 8-membered ring containing carbon atoms and optionally a heteroatom selected from O, S(O)ₓ, —NH, and —N(alkyl) and which is optionally substituted one or more times;
Ra^{a} and R^{b} are independently selected from the group consisting of hydrogen, CN, alkyl, haloalkyl, S(O)ₓNR¹⁰R¹¹, S(O)ₓR¹⁰ and C(O)NR¹⁰R¹¹, wherein alkyl and haloalkyl are optionally substituted one or more times;
E is selected from the group consisting of a bond, CR¹⁰R¹¹, O, NR⁵, S, S═O, S(═O)₂, C(═O), N(R¹⁰)(C═O), (C═O)N(R¹⁰), N(R¹⁰)S(═O)₂, S(═O)₂N(R¹⁰), C═N—OR¹¹, —C(R¹⁰R¹¹)C(R¹⁰R¹¹)—,—CH₂—W— and 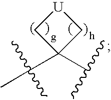
W is selected from the group consisting of O, NR⁵, S, S═O, S(═O)₂, N(R¹⁰)(C═O), N(R¹⁰)S(═O)₂ and S(═O )₂N(R¹⁰);
U is selected from the group consisting of C(R⁵R¹⁰), NR⁵, O, S, S═O and S(═O )₂;
g and h are independently selected from 0-2;
m and n are independently selected from 0-3, provided that:
(1) when E is present, m and n are not both 3;
(2) when E is —CH₂—W—, m and n are not 3; and
(3) when E is a bond, m and n are not 0;
p is selected from 0-6;
x is selected from 0-2;
wherein the dotted line represents optionally a double bond; and
N-oxides, pharmaceutically acceptable salts, and stereoisomers thereof.

25. A compound according to Formula (V): 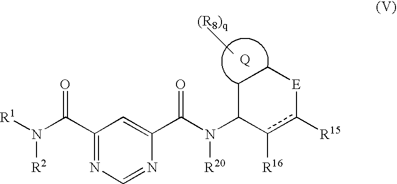
wherein:
R¹ is selected from the group consisting of alkyl, cycloalkyl-alkyl, arylalkyl, heteroarylalkyl and CHR²⁵R²¹, wherein alkyl, cycloalkyl-alkyl, arylalkyl and heteroarylalkyl are optionally substituted one or more times;
R² is hydrogen;
R⁴ is selected from the group consisting of R¹⁰, hydrogen, alkyl, aryl, heteroaryl, halo, CF₃, COR¹⁰, OR¹⁰, NR¹⁰R¹¹, NO₂, CN, SO₂OR¹⁰, CO₂R¹⁰, C(O)NR¹⁰R¹¹, SO₂NR¹⁰R¹¹, SO₂R¹⁰, OC(O)R¹⁰, OC(O)NR¹⁰R¹¹, NR¹⁰C(O)R¹¹, NR¹⁰CO₂R¹¹, (C₀-C₆)-alkyl-C(═NRa^{a})NHR^{b}, (C₀-C₆)-alkyl-NHC(═NR^{a})NHR^{b}, (C₀-C₆)-alkyl-C(O)OR¹⁰, (C₀-C₆)-alkyl-C(O)NR¹⁰R¹¹, (C₀-C₆)-alkyl-C(O)—NH—CN, O—(C₀-C₆)-alkyl-C(O)NR¹⁰R¹¹, S(O)ₓ—(C₀-C₆)-alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—SO₂NR¹⁰R¹¹, wherein each R⁴ group is optionally substituted by one or more R¹⁴ groups;
R⁵ is selected from the group consisting of hydrogen, alkyl, C(O)NR¹⁰R¹¹, aryl, arylalkyl, SO₂NR¹⁰R¹¹, C(O)OR¹⁰ and CN, wherein alkyl, aryl and arylalkyl are optionally substituted one or more times;
R⁸ is selected from the group consisting of hydrogen, alkyl, OR¹⁰, NR¹⁰R¹¹, CN, arylalkyl, cycloalkyl-alkyl, heteroarylalkyl, heterocyclylalkyl and halo, wherein alkyl, arylalkyl, cycloalkyl-alkyl, heteroarylalkyl and heterocyclylalkyl are optionally substituted one or more times;
R¹⁰ and R¹¹ are independently selected from the group consisting of hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or R¹⁰ and R¹¹ when taken together with the nitrogen to which they are attached complete a 3- to 8-membered ring containing carbon atoms and optionally containing a heteroatom selected from O, S, or NR⁵⁰ and which is optionally substituted one or more times;
R¹⁴ is selected from the group consisting of hydrogen, alkyl, arylalkyl, cycloalkyl-alkyl, heteroarylalkyl, heterocyclylalkyl and halo, wherein alkyl, arylalkyl, cycloalkyl-alkyl, heteroarylalkyl and heterocyclylalkyl are optionally substituted one or more times;
R¹⁵ and R¹⁶ when taken together with the carbon atoms to which they are bound, form a ring selected from the group consisting of 6-membered aryl ring, 5- or 6-membered heteroaryl ring, 5- to 8-membered cycloalkyl ring, 5- to 8-membered heterocyclyl ring, 5- to 8-membered cycloalkenyl ring and 5- to 8-membered heterocycloalkenyl ring, wherein said ring is optionally substituted by one or more R⁴ groups;
R²⁰ is selected from the group consisting of hydrogen and alkyl, wherein alkyl is optionally substituted one or more times;
R²¹ is a bicyclic or tricyclic fused ring system, wherein at least one ring is partially saturated, and wherein said bicyclic or tricyclic fused ring system is optionally substituted one or more times;
R²⁵ is selected from the group consisting of hydrogen, alkyl, cycloalkyl, C(O)NR¹⁰R¹¹ and haloalkyl, wherein alkyl, cycloalkyl, and haloalkyl are optionally substituted one or more times;
R⁵⁰ is selected from the group consisting of hydrogen, alkyl, aryl, heteroaryl, C(O)R⁸⁰, C(O)NR⁸⁰R⁸¹, SO₂R⁸⁰ and SO₂NR⁸⁰R⁸¹, wherein alkyl, aryl and heteroaryl are optionally substituted one or more times;
R⁸⁰ and R⁸¹ are independently selected from the group consisting of hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or R⁸⁰ and R⁸¹ when taken together with the nitrogen to which they are attached complete a 3- to 8-membered ring containing carbon atoms and optionally a heteroatom selected from O, S(O)ₓ, —NH, and —N(alkyl) and which is optionally substituted one or more times;
Ra^{a} and R^{b} are independently selected from the group consisting of hydrogen, CN, alkyl, haloalkyl, S(O)ₓNR¹⁰R¹¹, S(O)ₓR¹⁰ and C(O)NR¹⁰R¹¹, wherein alkyl and haloalkyl are optionally substituted one or more times;
E is selected from the group consisting of a bond, CR¹⁰R¹¹, O, NR⁵, S, S═O, S(═O)₂, C(═O), N(R¹⁰)(C═O), (C═O)N(R¹⁰), N(R¹⁰)S(═O)₂, S(═O)₂N(R¹⁰), C═N—OR¹¹, —C(R¹⁰R¹¹)C(R¹⁰R¹¹)—,—CH₂—W— and 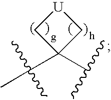
W is selected from the group consisting of O, NR⁵, S, S═O, S(═O)₂, N(R¹⁰)(C═O), N(R¹⁰)S(═O)₂ and S(═O)₂N(R¹⁰);
U is selected from the group consisting of C(R⁵R¹⁰), NR⁵, O, S, S═O and S(═O)₂;
Q is selected from the group consisting of 3-7 membered cycloalkyl, 4-7 membered heterocyclyl, 5-6 membered heteroaryl and 6 membered aryl;
g and h are independently selected from 0-2;
q is selected from 0-4;
x is selected from 0-2;
wherein the dotted line represents optionally a double bond; and
N-oxides, pharmaceutically acceptable salts, and stereoisomers thereof.

26. The compound according to claim 1, selected from the group consisting of:

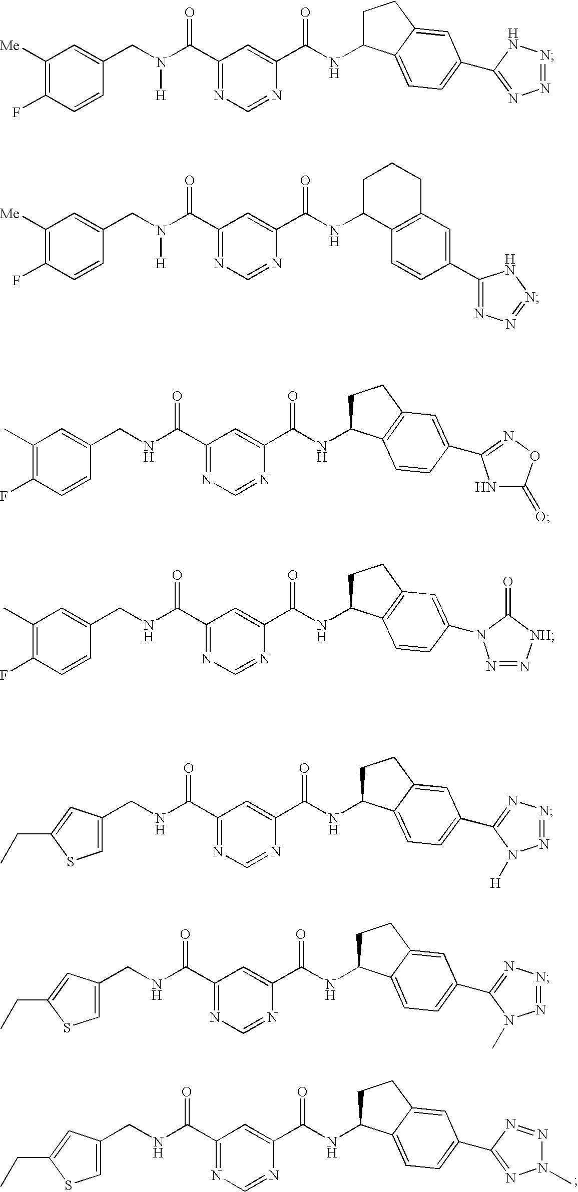

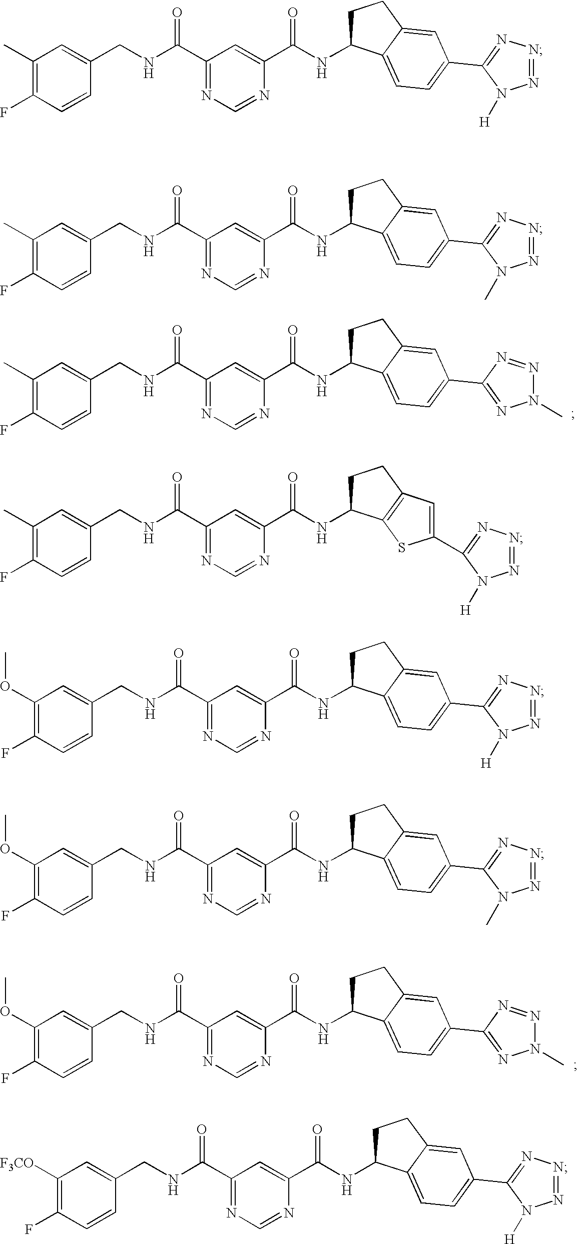

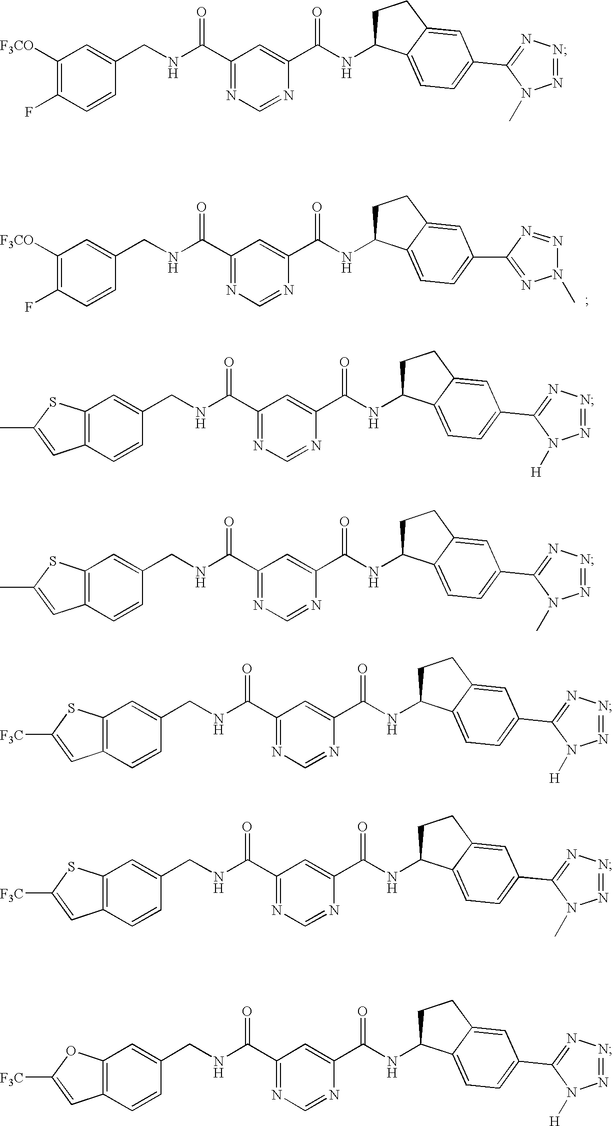

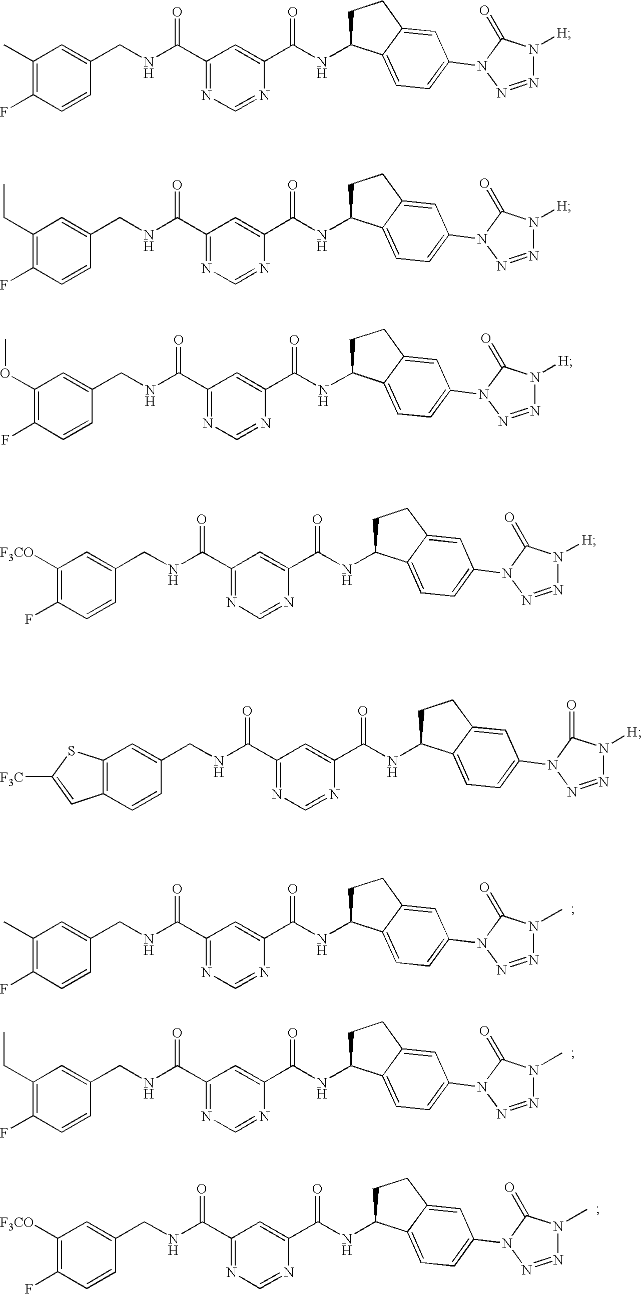

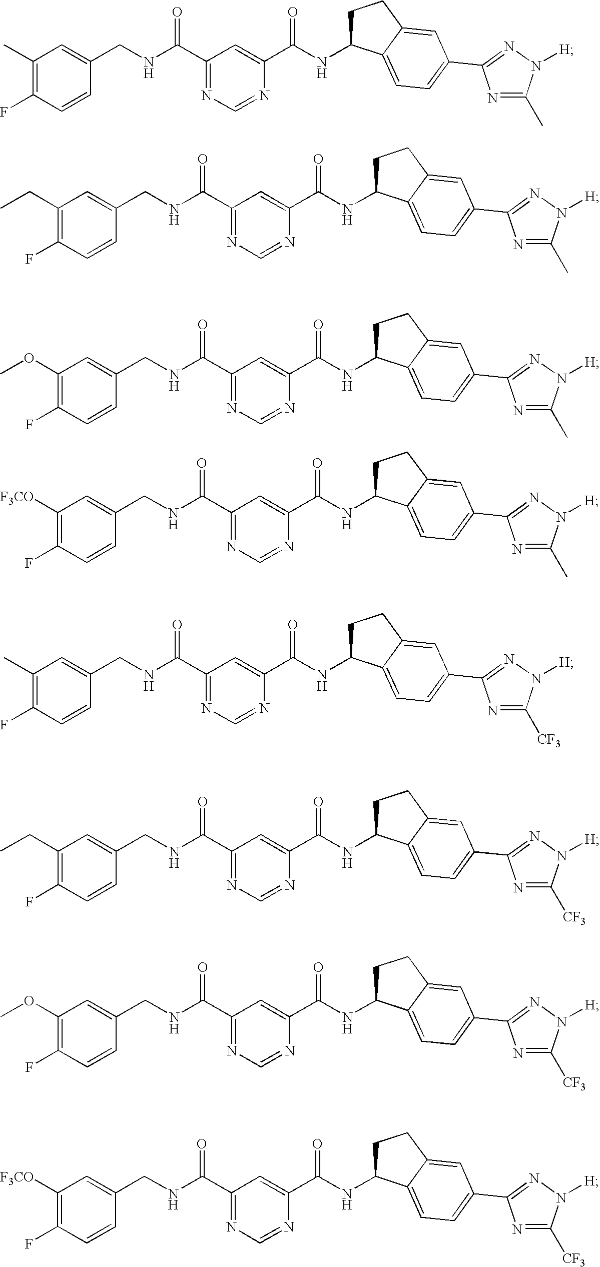

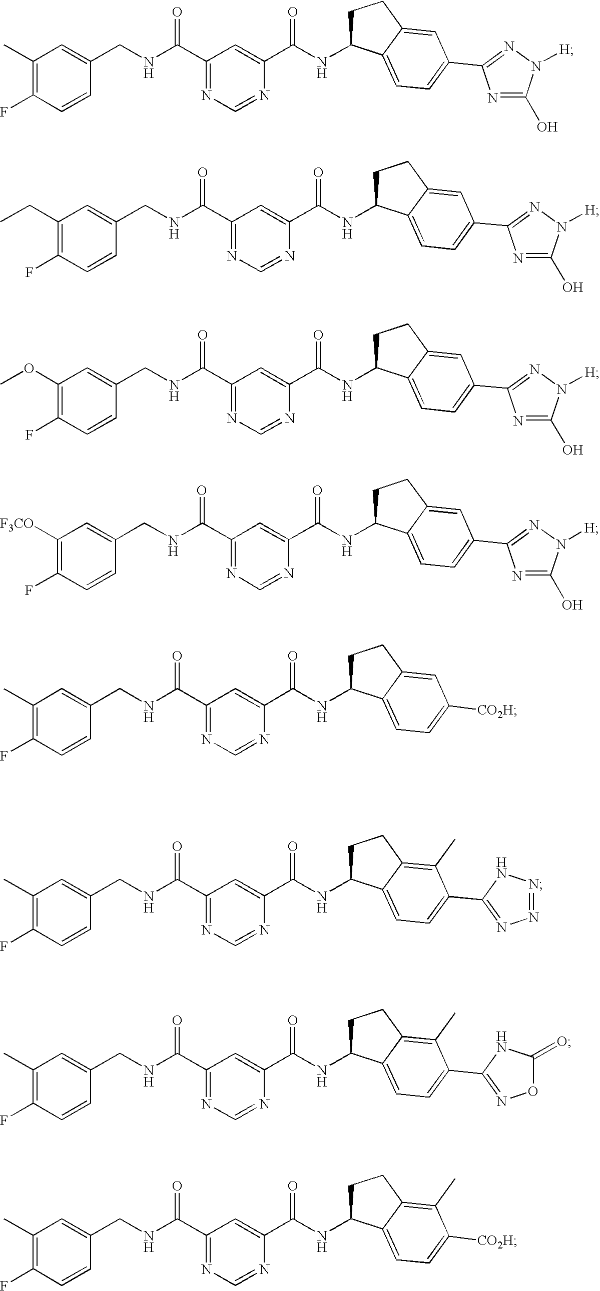

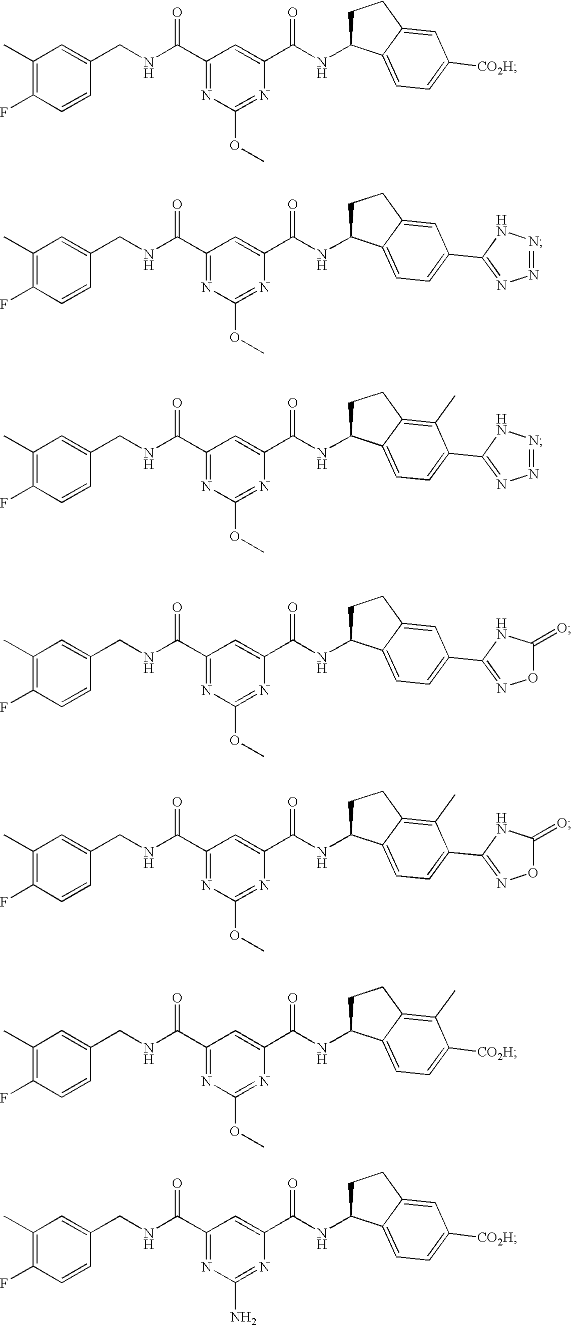

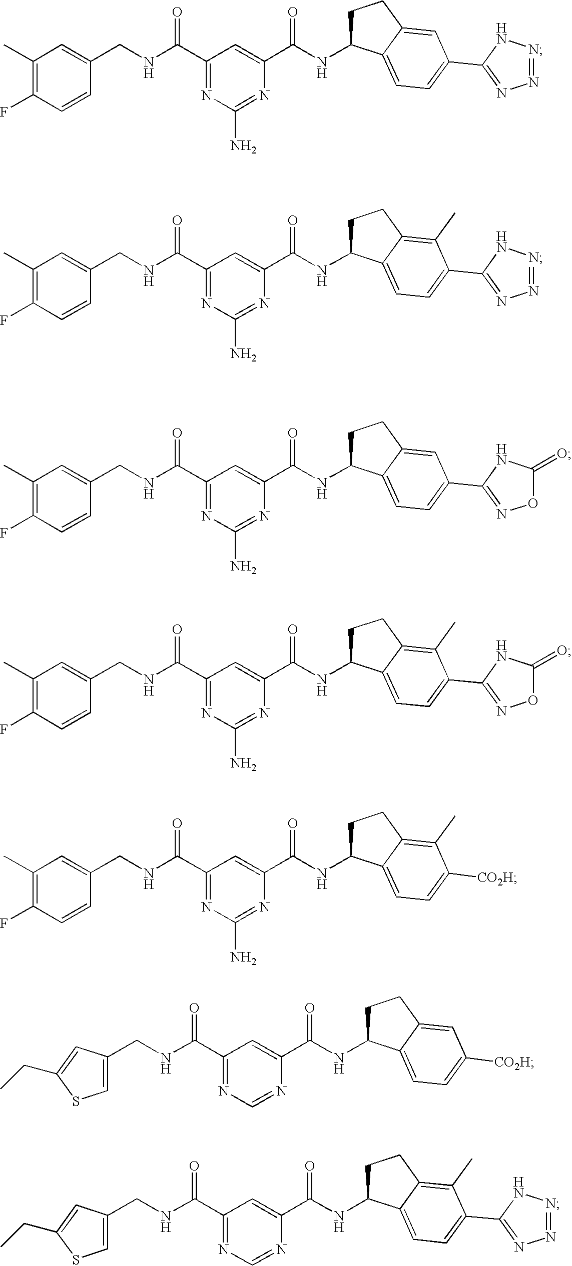

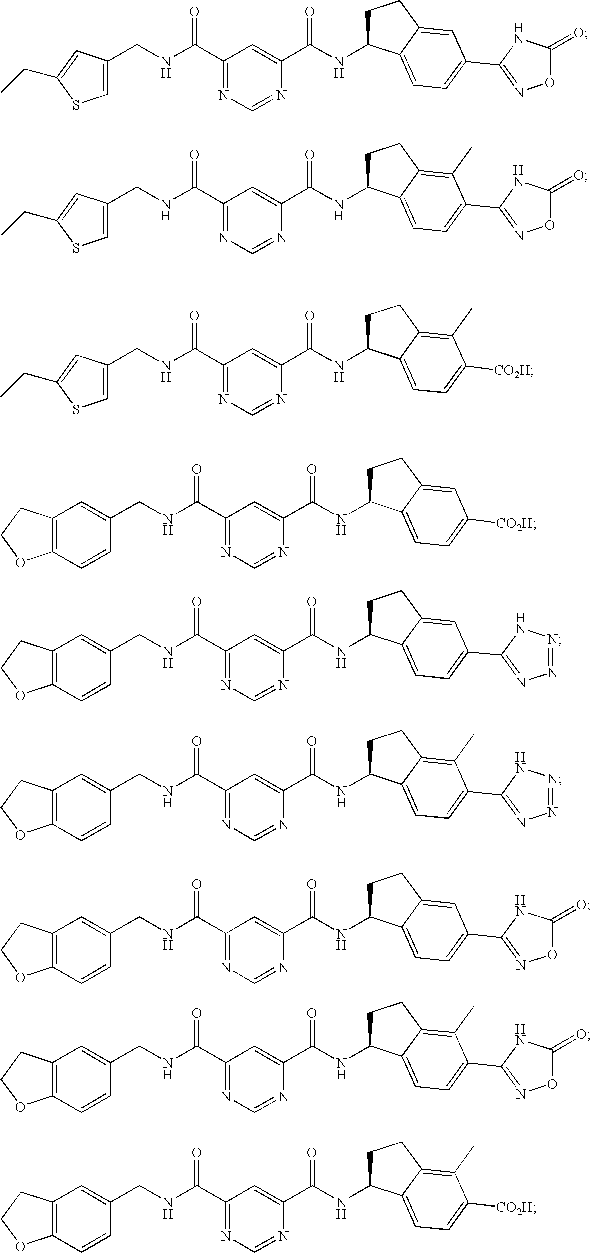

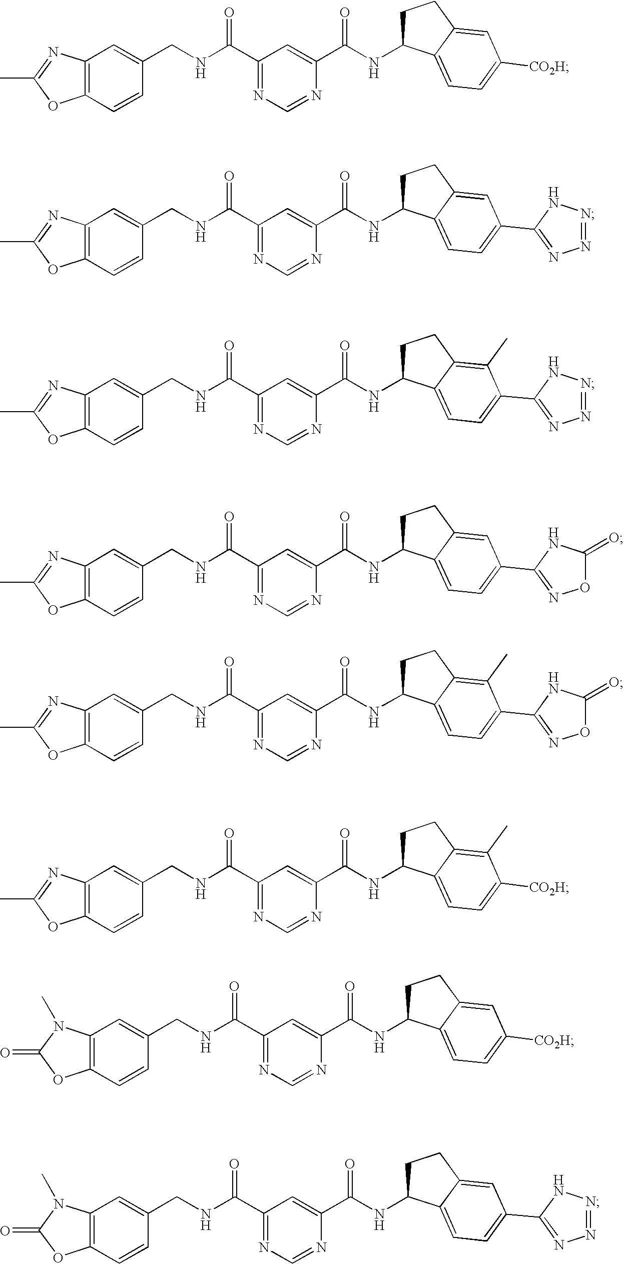

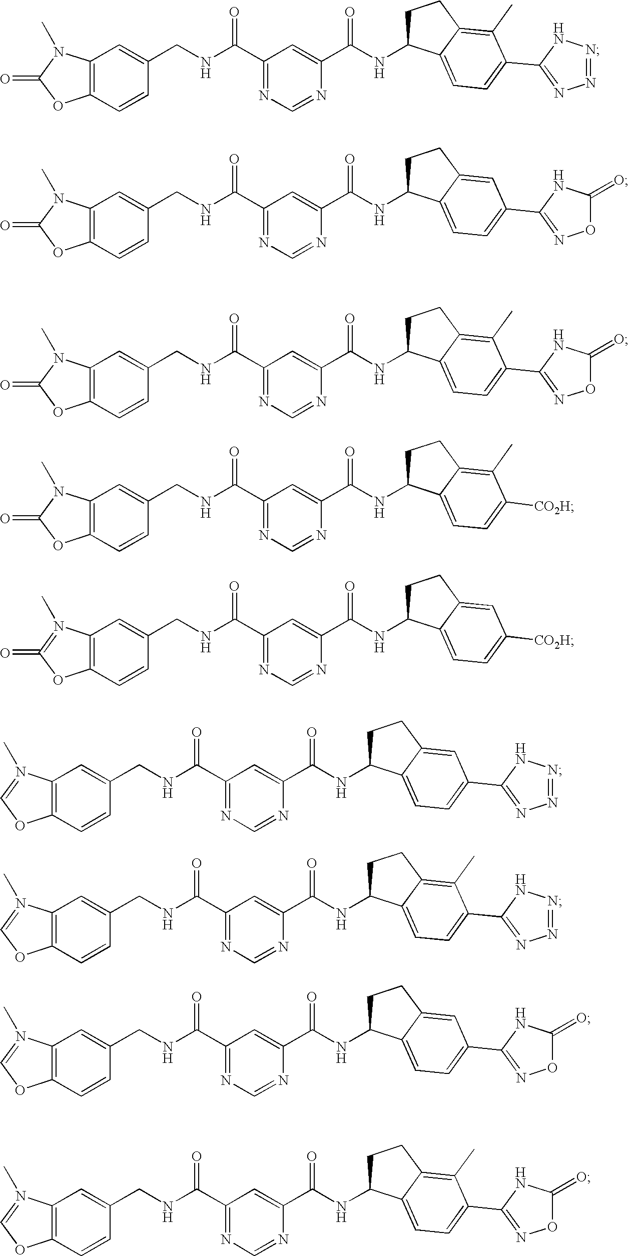

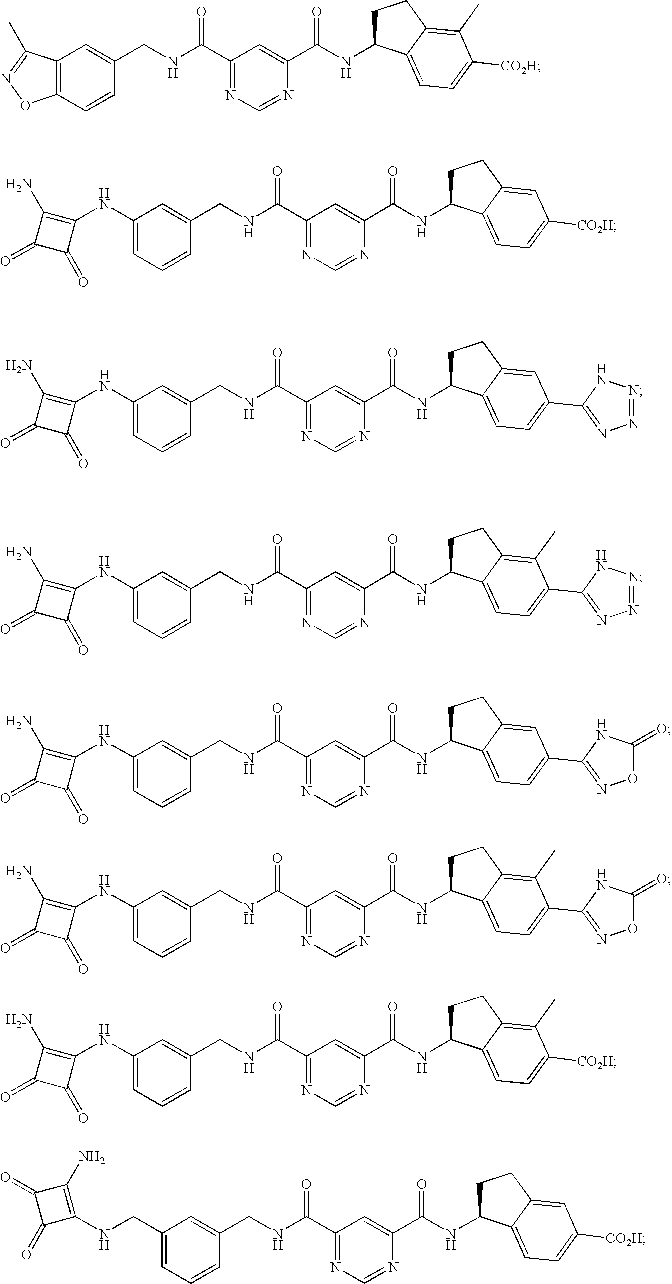

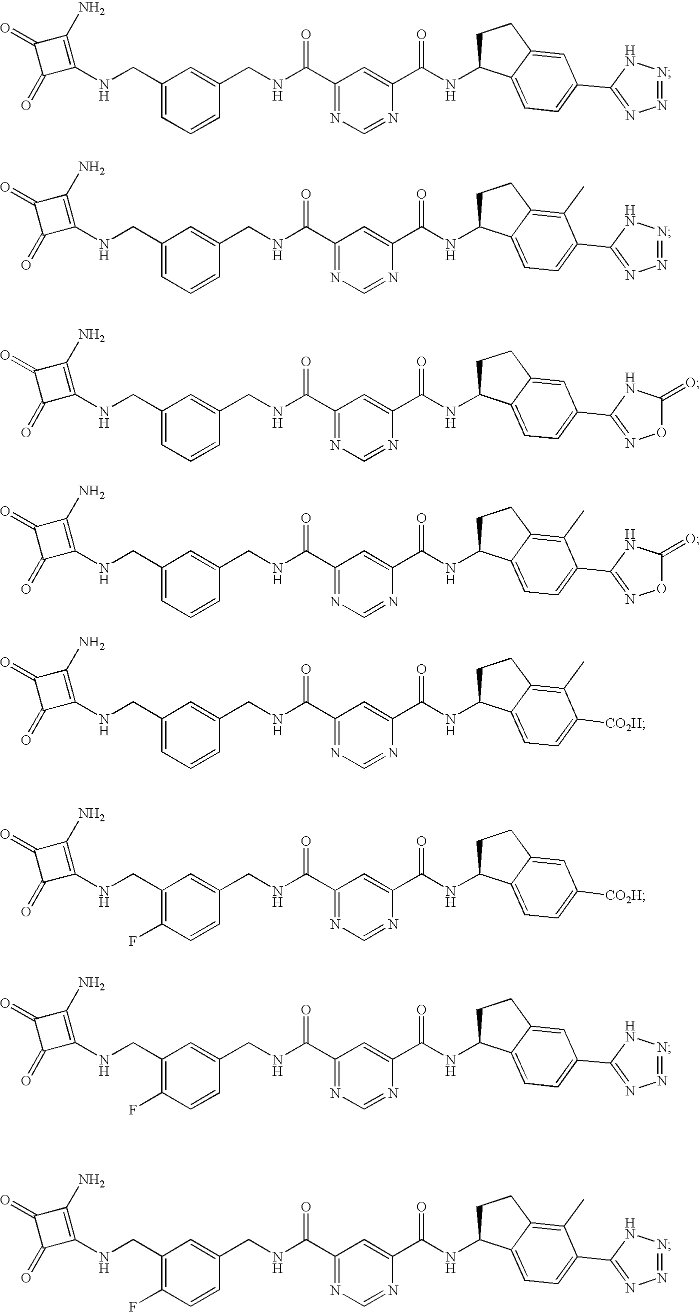

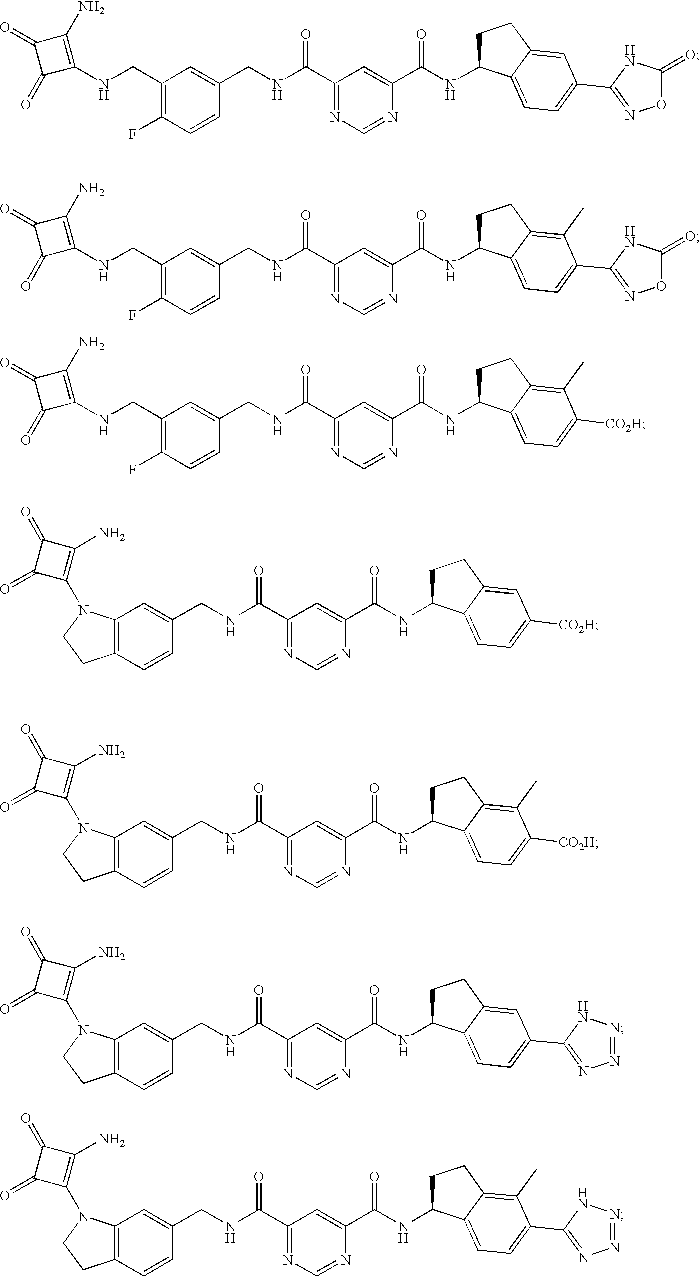

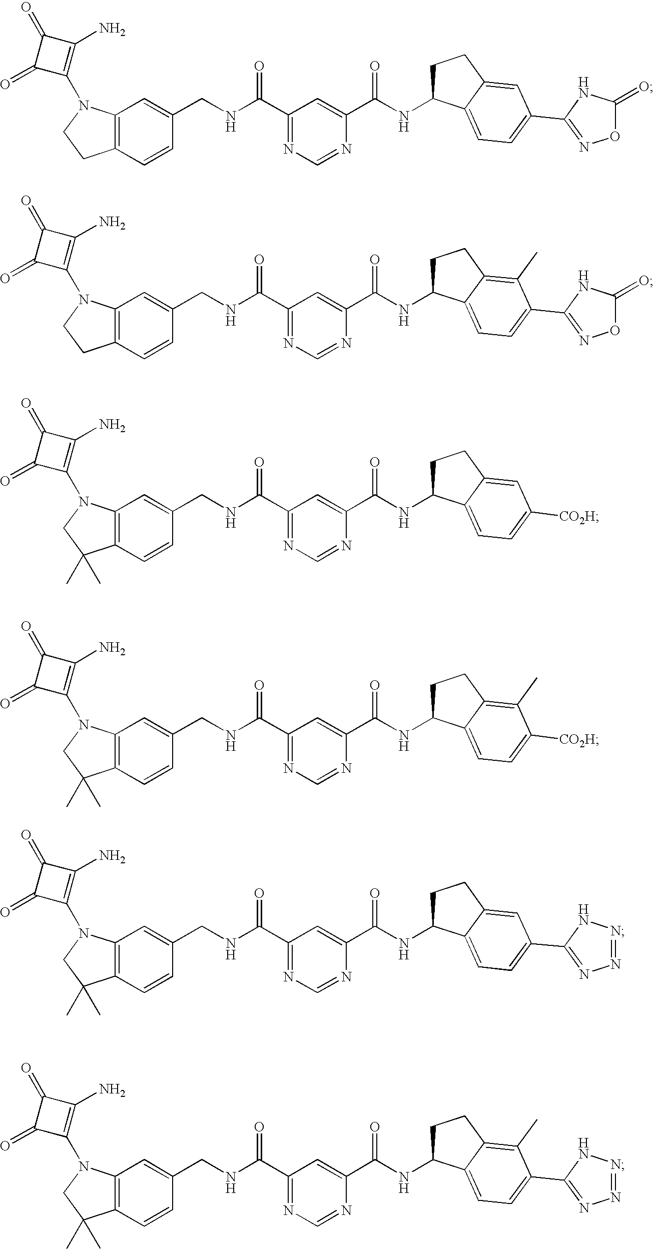

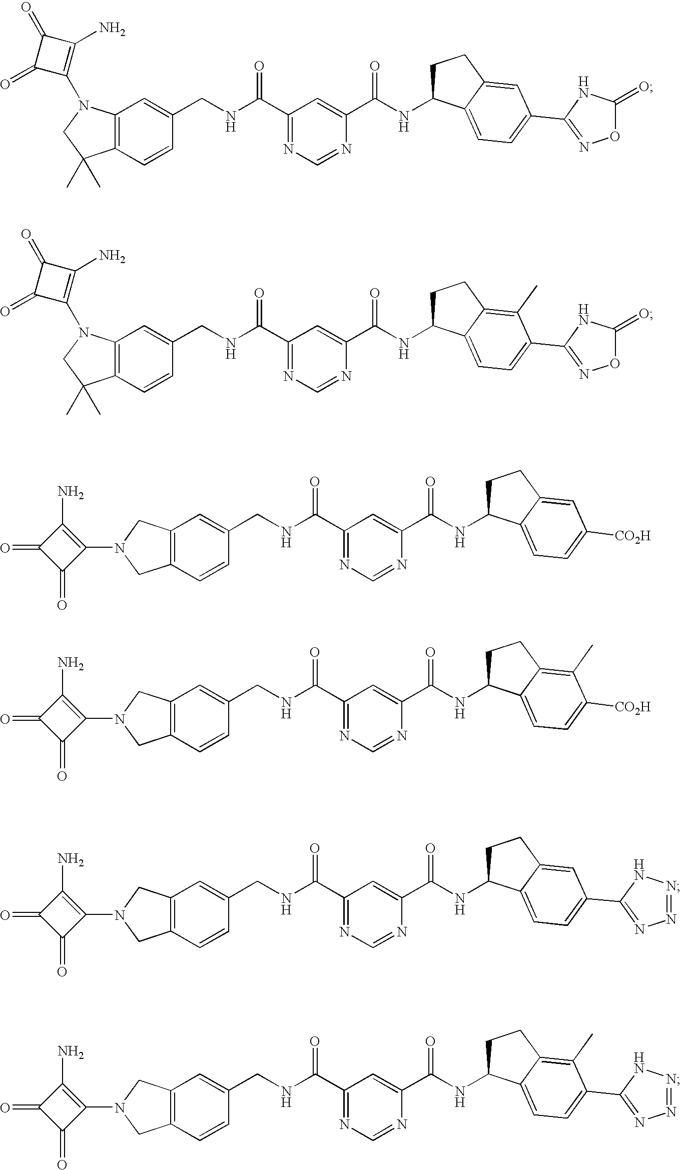

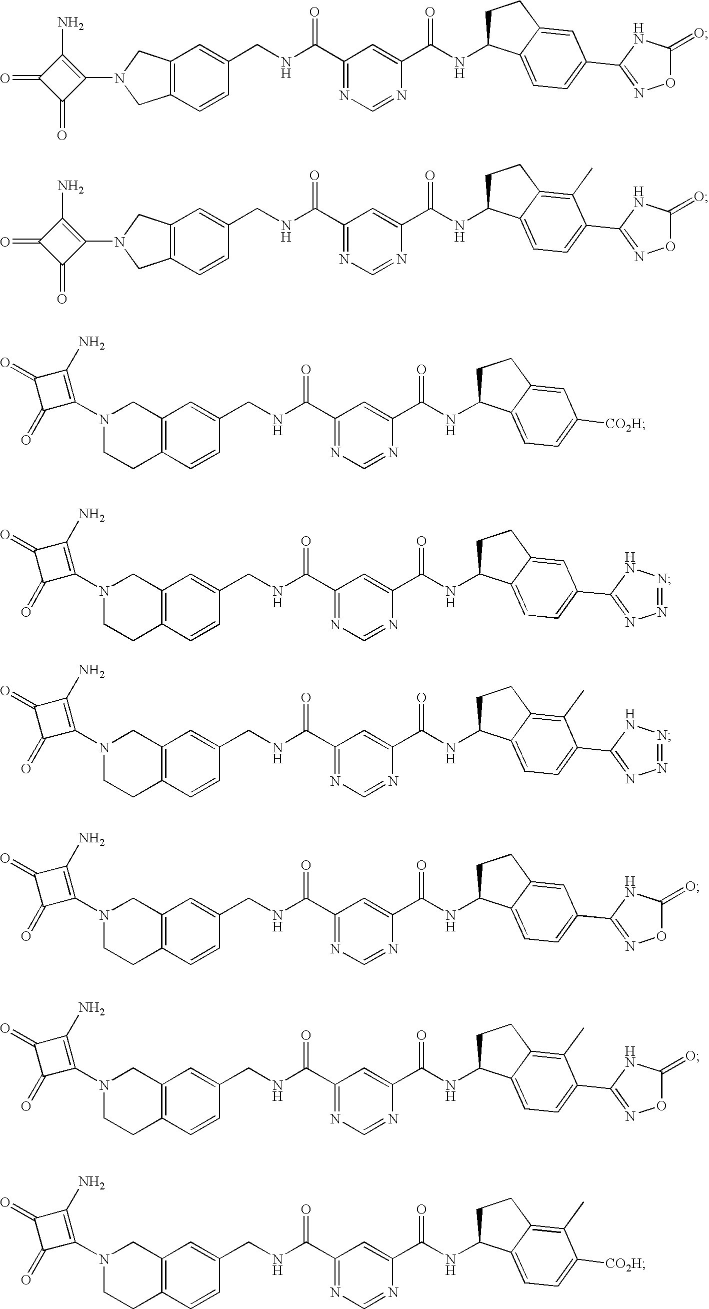

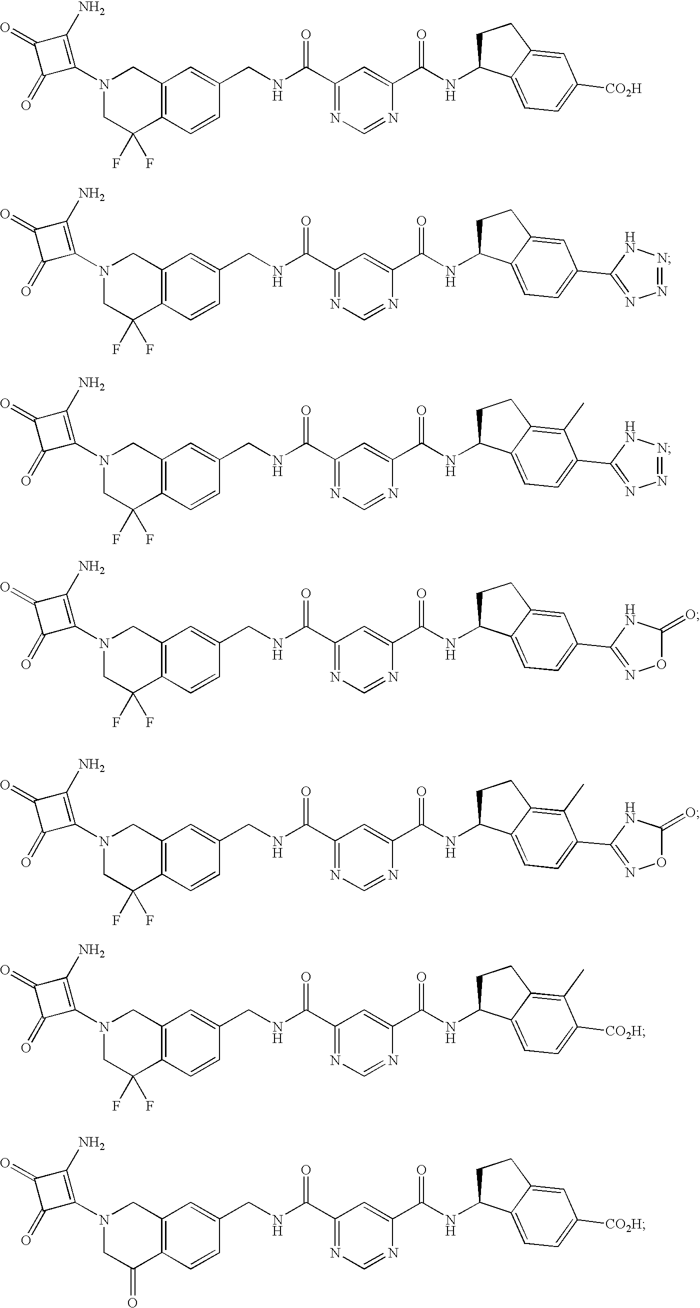

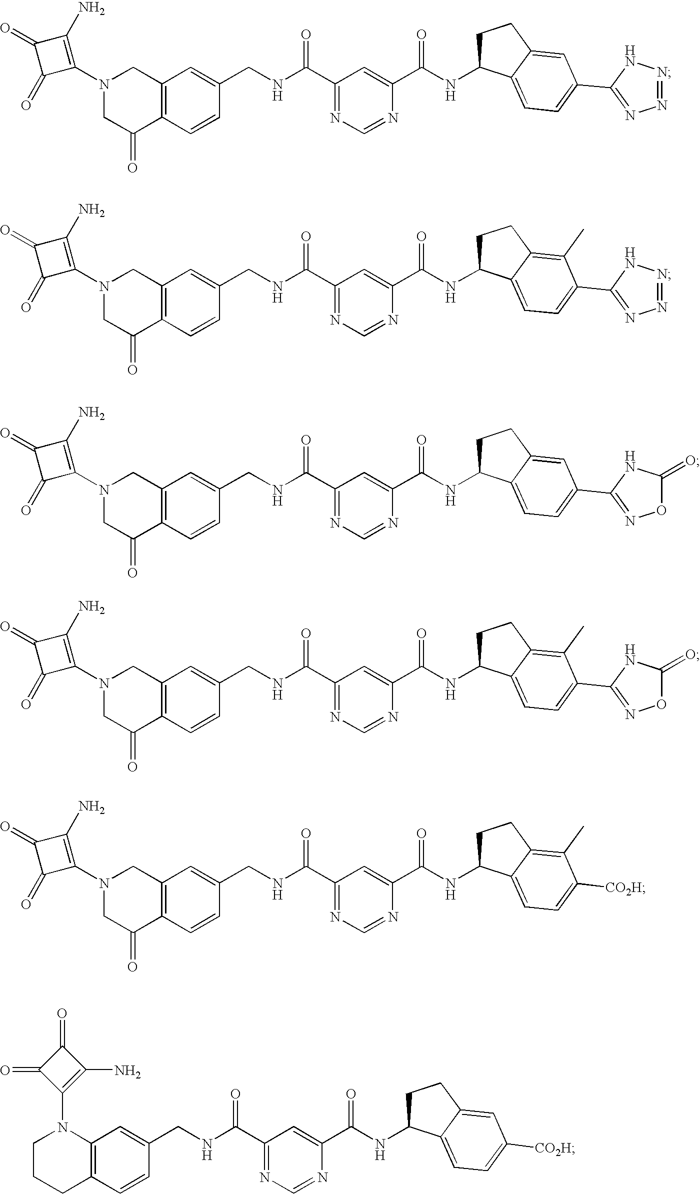

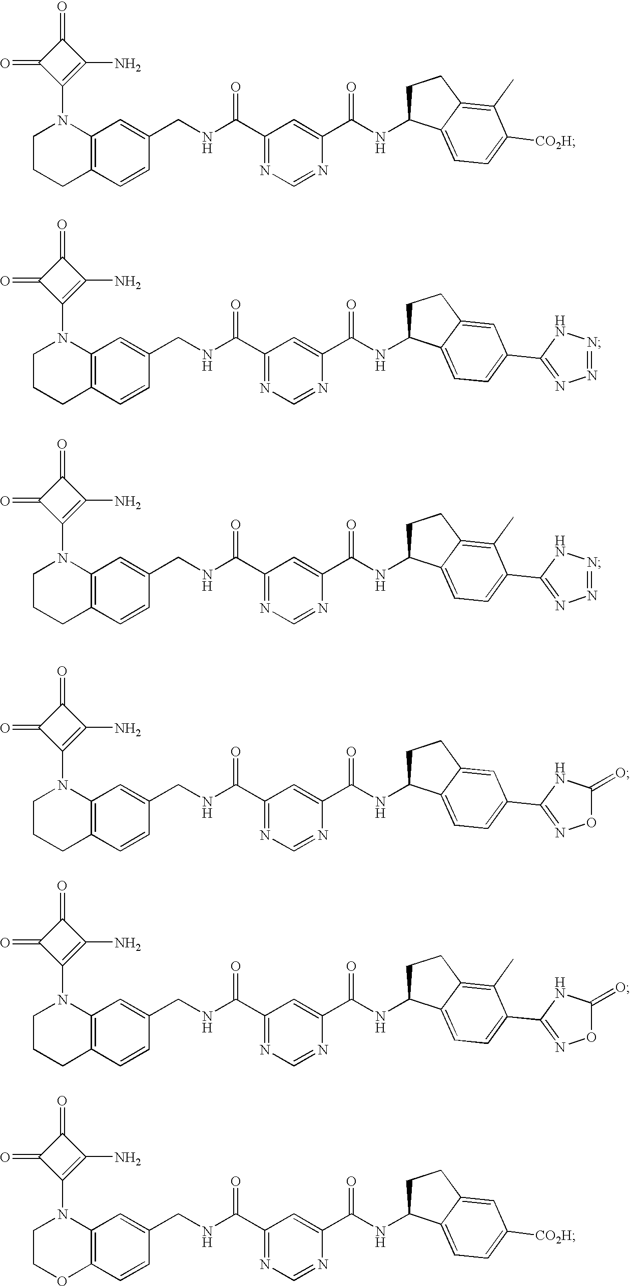

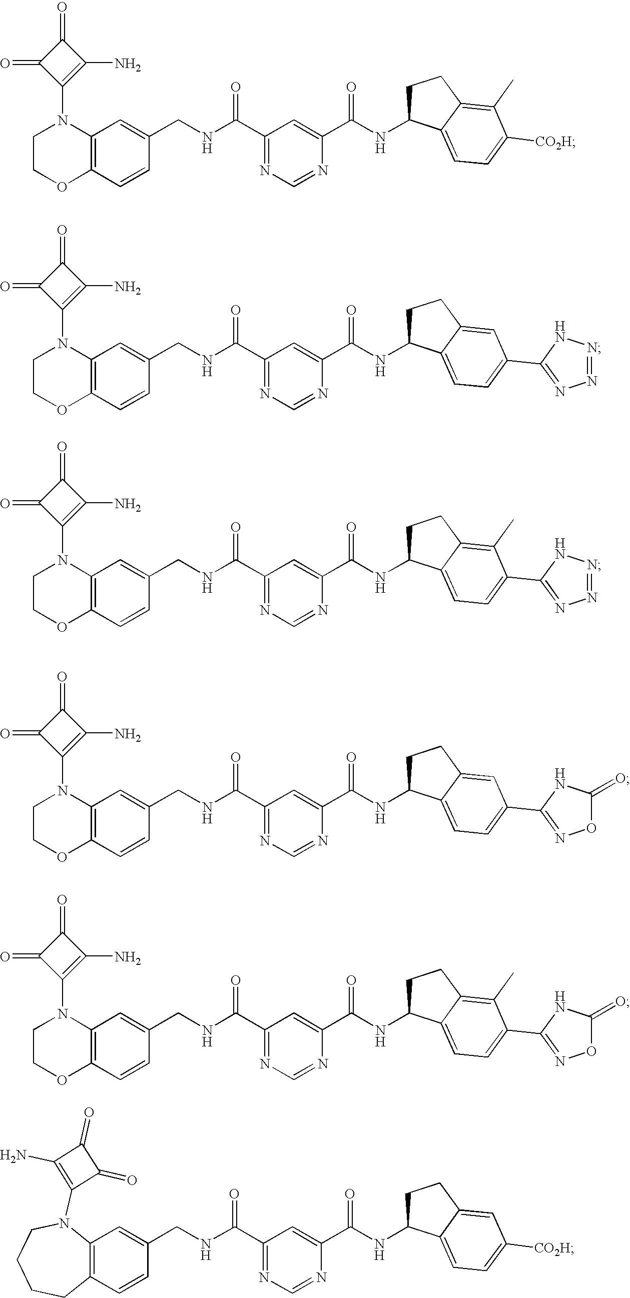

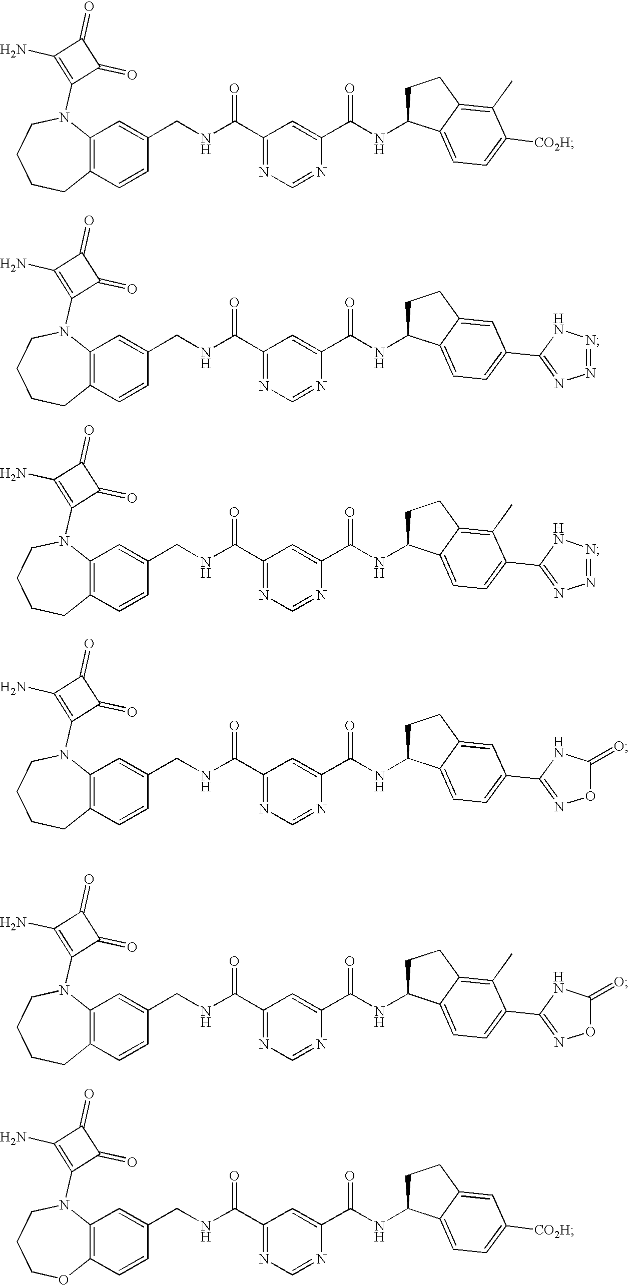

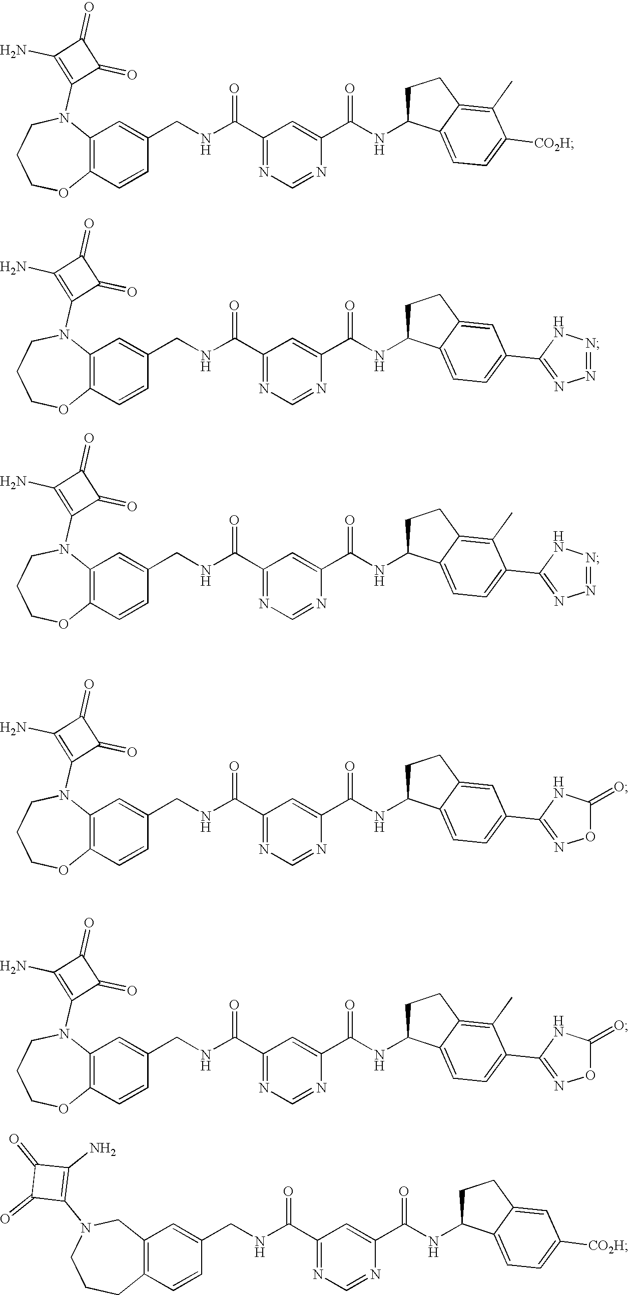

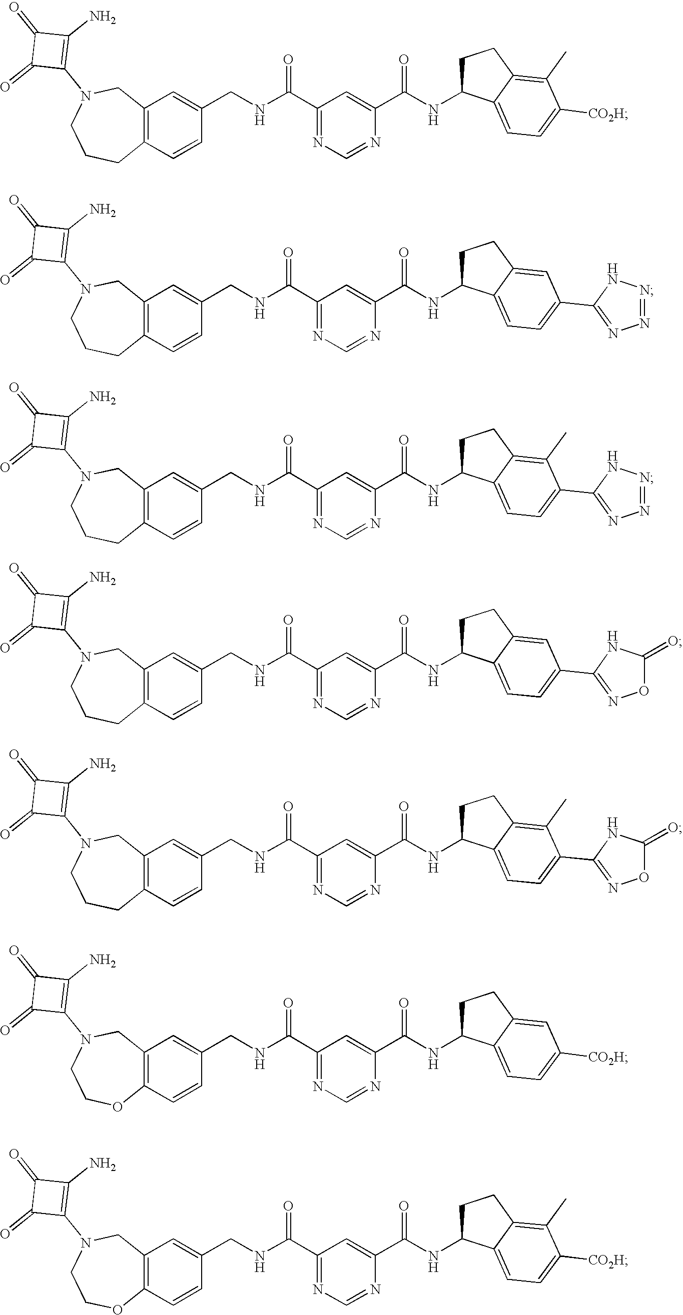

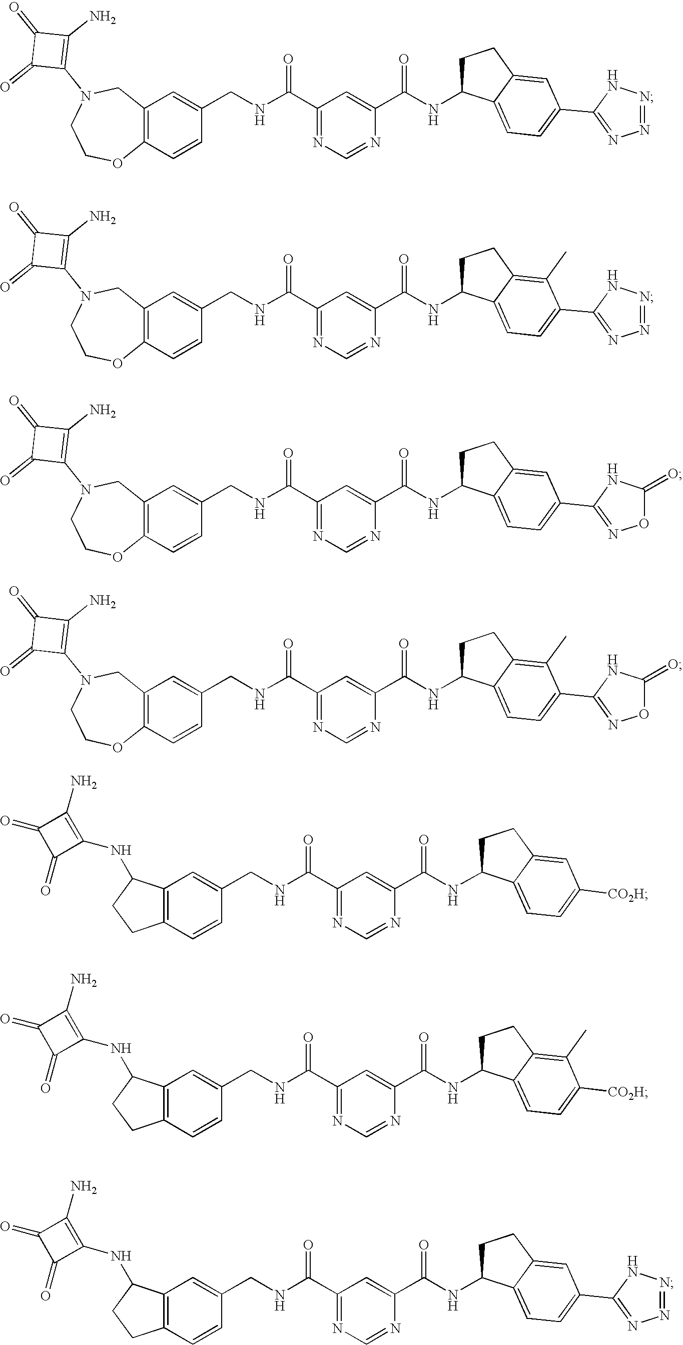

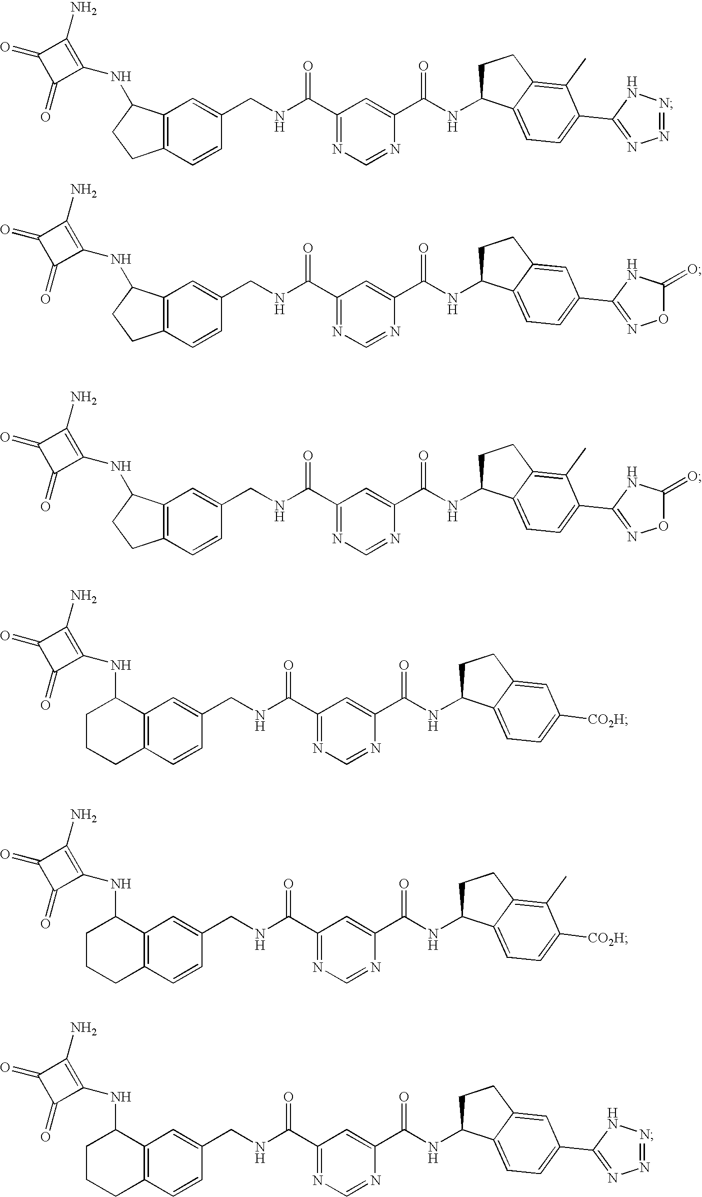

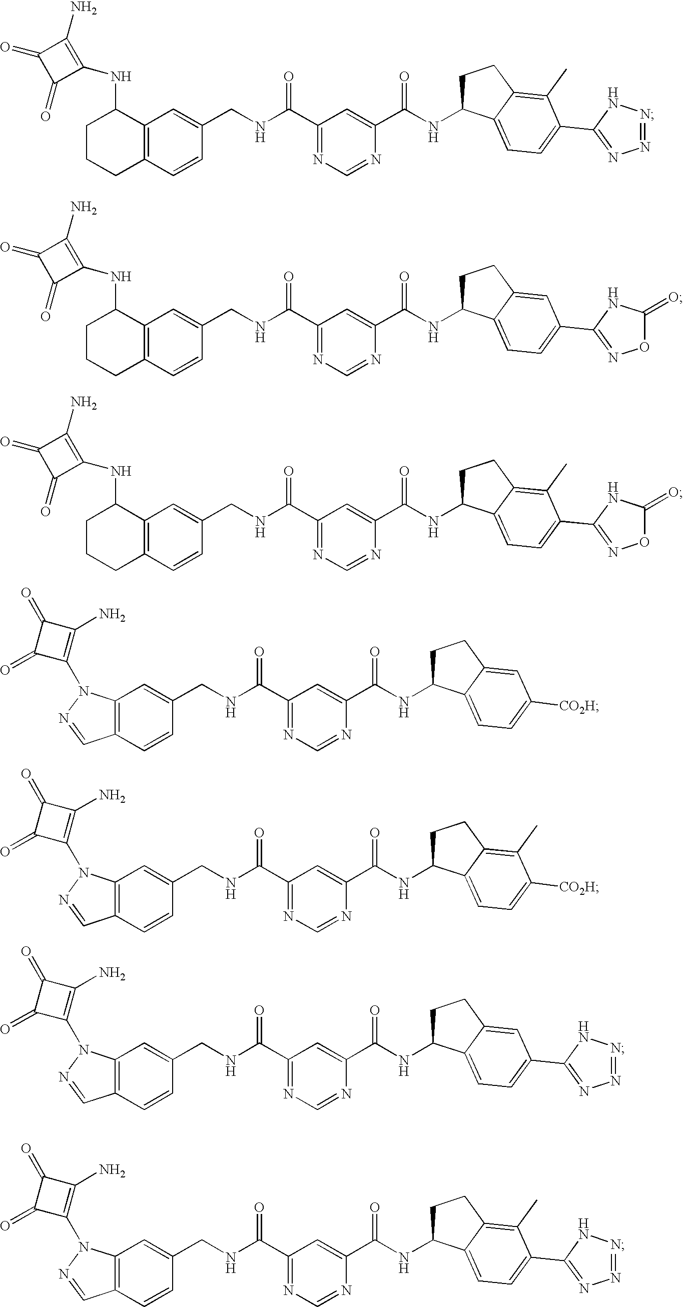

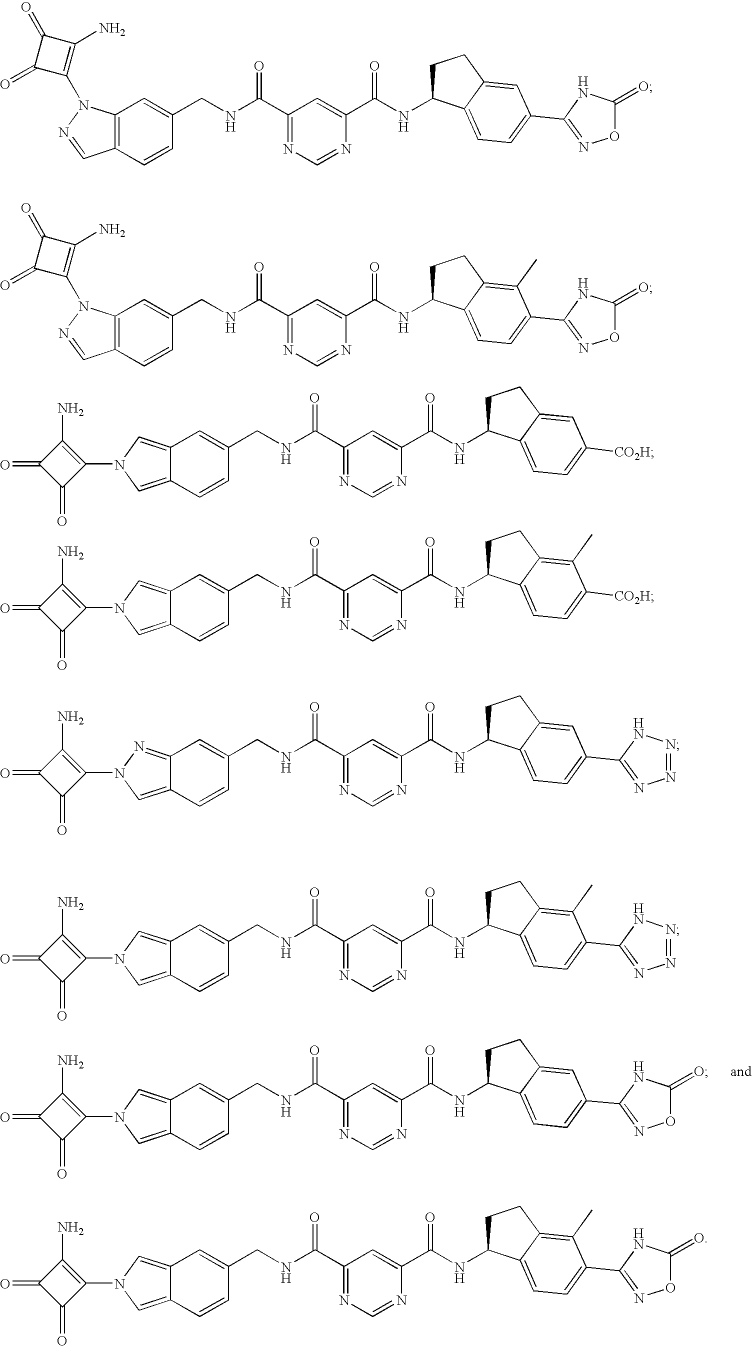

27. The compound according to claim 1, which comprises:

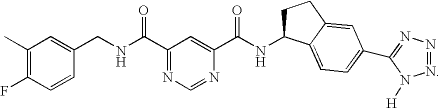

28. The compound according to claim 1, which comprises:

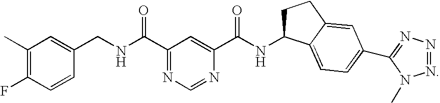

29. The compound according to claim 1, which comprises:

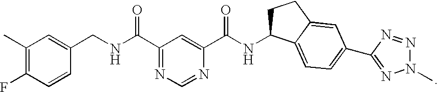

30. The compound according to claim 1, which comprises:

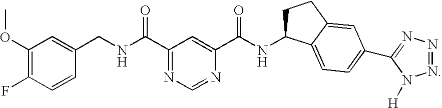

31. The compound according to claim 1, which comprises:

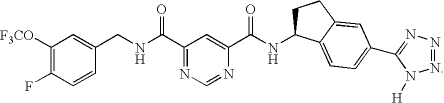

32. The compound according to claim 1, which comprises:

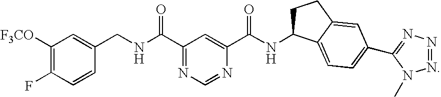

33. The compound according to claim 1, which comprises:

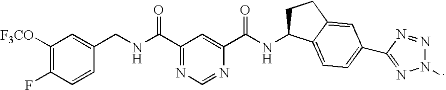

34. The compound according to claim 1, which comprises:

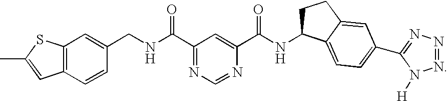

35. The compound according to claim 1, which comprises:

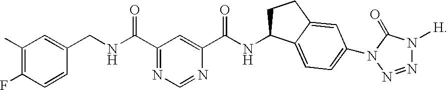

36. The compound according to claim 1, which comprises:

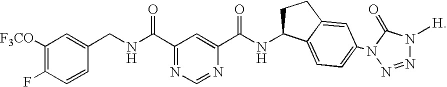

37. The compound according to claim 1, which comprises:

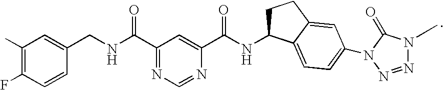

38. The compound according to claim 1, which comprises:

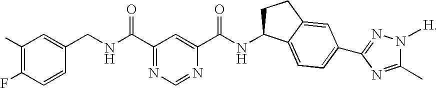

39. The compound according to claim 1, which comprises:

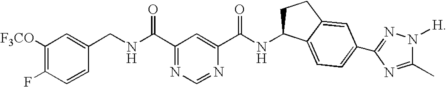

40. The compound according to claim 1, which comprises:

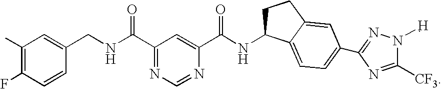

41. The compound according to claim 1, which comprises:

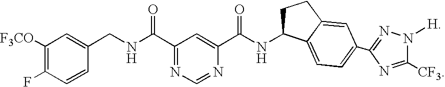

42. The compound according to claim 1, which comprises:

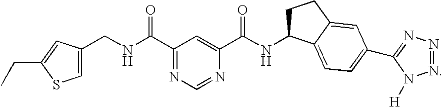

43. The compound according to claim 1, which comprises:

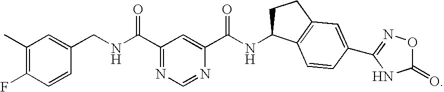

44. The compound according to claim 1, which comprises:

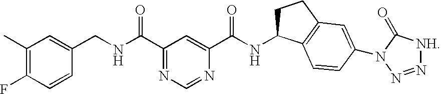

45. The compound according to claim 1, which comprises:

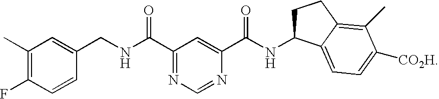

46. The compound according to claim 1, which comprises:

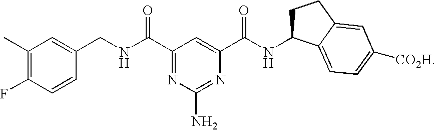

47. The compound according to claim 1, which comprises:

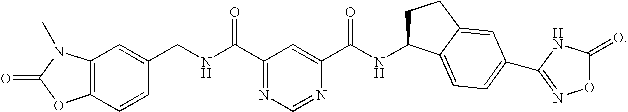

48. The compound according to claim 1, which comprises:

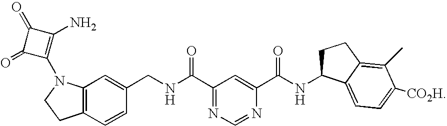

49. The compound according to claim 1, which comprises:

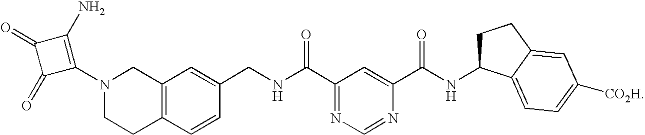

50. The compound according to claim 1, which comprises:

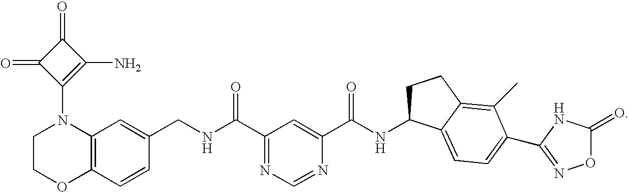

51. The compound according to claim 1, which comprises:

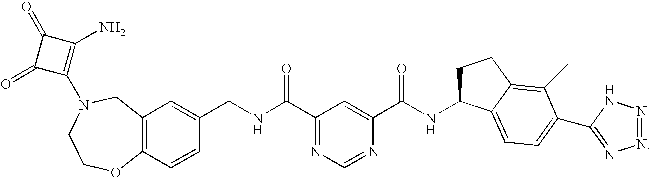

52. A compound according to Formula (VI): 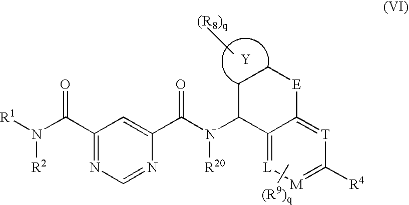
wherein:
R¹ is selected from the group consisting of alkyl, cycloalkyl-alkyl, arylalkyl, heteroarylalkyl and CHR²⁵R²¹, wherein alkyl, cycloalkyl-alkyl, arylalkyl and heteroarylalkyl are optionally substituted one or more times;
R² is hydrogen;
R⁴ is selected from the group consisting of R¹⁰, hydrogen, alkyl, aryl, heteroaryl, halo, CF₃, COR¹⁰, OR¹⁰, NR¹⁰R¹¹, NO₂, CN, SO₂OR¹⁰, CO₂R¹⁰, C(O)NR¹⁰R¹¹, SO₂NR¹⁰R¹¹, SO₂R¹⁰, OC(O)R¹⁰, OC(O)NR¹⁰R¹¹, NR¹⁰C(O)R¹¹, NR¹⁰CO₂R¹¹, (C₀-C₆)-alkyl-C(═NR^{a})NHR^{b}, (C₀-C₆)-alkyl-NHC(═NR^{a})NHR^{b}, (C₀-C₆)-alkyl-C(O)OR¹⁰, (C₀-C₆)-alkyl-C(O)NR¹⁰R¹¹, (C₀-C₆)-alkyl-C(O)—NH—CN, O—(C₀-C₆)-alkyl-C(O)NR¹⁰R¹¹, S(O)ₓ—(C₀-C₆)-alkyl-C(O)OR¹⁰, S(O)ₓ—(C₀-C₆)-alkyl0C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—SO₂NR¹⁰R¹¹, wherein each R⁴ group is optionally substituted by one or more R¹⁴ groups;
R⁵ is selected from the group consisting of hydrogen, alkyl, C(O)NR¹⁰R¹¹, aryl, arylalkyl, SO₂NR¹⁰R¹¹, C(O)OR¹⁰ and CN, wherein alkyl, aryl and arylalkyl are optionally substituted one or more times;
R⁸ is selected from the group consisting of hydrogen, alkyl, OR¹⁰, NR¹⁰R¹¹, CN, arylalkyl, cycloalkyl-alkyl, heteroarylalkyl, heterocyclylalkyl and halo, wherein alkyl, arylalkyl, cycloalkyl-alkyl, heteroarylalkyl and heterocyclylalkyl are optionally substituted one or more times;
R⁹ is selected from the group consisting of hydrogen, alkyl, CH(CH₃)CO₂H, halo, (C₀-C₆)-alkyl-C(O)OR¹⁰, (C₀-C₆)-alkyl-C(O)NR¹⁰R¹¹, (C₀-C₆)-alkyl-C(O)NH—CN, O—(C₀-C₆)-alkyl-C(O)NR¹⁰R¹¹, S(O)_{y}-alkyl-C(O)OR¹⁰, S(O)_{z}-alkyl-C(O)NR¹⁰R¹¹, (C₀-C₆)-alkyl-C(O)NR¹⁰—(C₀-C₆)-alkyl-NR¹⁰R¹¹, C(O)NR¹⁰—(C₀-C₆)-alkyl-heteroaryl, C(O)NR¹⁰—(C₀-C₆)-alkyl-aryl, CH₂NR¹⁰R¹¹, (CH₂)_{y}NR¹⁰C(O)-alkyl, (CH₂)_{w}NR¹⁰C(O)—(C₀-C₆)-alkyl-aryl, (CH₂)_{w}NR¹⁰C(O)—(C₀-C₆)-alkyl-heteroaryl, (CH₂)_{w}NR¹⁰C(O)O-alkyl, (CH₂)_{w}NR¹⁰C(O)O—(C₀-C₆)-alkyl-aryl, (CH₂)_{w}NR¹⁰C(O)O—(C₀-C₆)-alkyl-heteroaryl, (CH₂)_{w}NR¹⁰C(O)ONR¹⁰R¹¹, (CH₂)_{w}NR¹⁰S(O)₂—(C₀-C₆)-alkyl-aryl, (CH₂)_{w}NR¹⁰S(O)₂—(C₀-C₆)-alkyl-heteroaryl, (CH₂)_{w}NR¹⁰S(O)₂—NR¹⁰-akkyl, (CH₂)_{w}NR¹⁰S(O)₂NR¹⁰—(C₀-C₆)-alkyl-aryl, (CH₂)_{w}NR¹⁰S(O)₂NR¹⁰—(C₀-C₆)-alkyl-heteroaryl (CH₂)_{w}NR¹⁰C(O)NR¹⁰—SO₂—R³⁰ , S(O)₂NR¹⁰—(C₀-C₆)-alkyl-aryl, S(O)₂NR¹⁰—(C₀C₆)-alkyl-heteroaryl, S(O)₂NR¹⁰-alkyl, S(O)₂—(C₀-C₆)-alkyl-aryl, S(O)₂—(C₀-C₆)-alkyl-heteroaryl, O-heteroaryl and heteroaryl, wherein each of said R⁹ groups is optionally substituted one or more times;
R¹⁰ and R¹¹ are independently selected from the group consisting of hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or R¹⁰ and R¹¹ when taken together with the nitrogen to which they are attached complete a 3- to 8-membered ring containing carbon atoms and optionally containing a heteroatom selected from O, S, or NR⁵⁰ and which is optionally substituted one or more times;
R¹⁴ is selected from the group consisting of hydrogen, alkyl, arylalkyl, cycloalkyl-alkyl, heteroarylalkyl, heterocyclylalkyl and halo, wherein alkyl, arylalkyl, cycloalkyl-alkyl, heteroarylalkyl and heterocyclylalkyl are optionally substituted one or more times;
R²⁰ is selected from the group consisting of hydrogen and alkyl, wherein alkyl is optionally substituted one or more times;
R²¹ is a bicyclic or tricyclic fused ring system, wherein at least one ring is partially saturated, and wherein said bicyclic or tricyclic fused ring system is optionally substituted one or more times;
R²⁵ is selected from the group consisting of hydrogen, alkyl, cycloalkyl, C(O)NR¹⁰R¹¹ and haloalkyl, wherein alkyl, cycloalkyl, and haloalkyl are optionally substituted one or more times;
R³⁰ is selected from the group consisting of alkyl and (C₀-C₆)-alkyl-aryl;
R⁵⁰ is selected from the group consisting of hydrogen, alkyl, aryl, heteroaryl, C(O)R⁸⁰, C(O)NR⁸⁰R⁸¹, SO₂R⁸¹ and SO₂NR⁸⁰R⁸¹, wherein alkyl, aryl and heteroaryl are optionally substituted one or more times;
R⁸⁰ and R⁸¹ are independently selected from the group consisting of hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or R⁸⁰ and R⁸¹ when taken together with the nitrogen to which they are attached complete a 3- to 8-membered ring containing carbon atoms and optionally a heteroatom selected from O, S(O)ₓ, —NH, and —N(alkyl) and which is optionally substituted one or more times;
Ra^{a} and R^{b} are independently selected from the group consisting of hydrogen, CN, alkyl, haloalkyl, S(O)ₓNR¹⁰R¹¹, S(O)ₓR¹⁰ and C(O)NR¹⁰R¹¹, wherein alkyl and haloalkyl are optionally substituted one or more times;
E is selected from the group consisting of a bond, CR¹⁰R¹¹, O, NR⁵, S, S═O, S(═O)₂, C(═O), N(R¹⁰)(C═O), (C═O)N(R¹⁰), N(R¹⁰)S(═O )₂, S(═O )₂N(R¹⁰), C═N—OR¹¹, —C(R¹⁰R¹¹)C(R¹⁰R¹¹)—, —CH₂—W— and 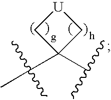
W is selected from the group consisting of O, NR⁵, S, S═O, S(═O)₂, N(R¹⁰)(C═O), N(R¹⁰)S(═O )₂ and S(═O )₂N(R¹⁰);
U is selected from the group consisting of C(R⁵R¹⁰), NR⁵, O, S, S═O and S(═O)₂;
Y is absent or selected from the group consisting of 3-7 membered cycloalkyl, 4-7 membered heterocyclyl, 5-6 membered heteroaryl and 6-membered aryl;
L, M and T are independently selected from the group consisting of C and N;
g and h are independently selected from 0-2;
q is selected from 0-4;
w is selected from 0-4;
x is selected from 0-2;
y is selected from 1 and 2;
z is selected from 0-2; and
N-oxides, pharmaceutically acceptable salts, and stereoisomers thereof.

53. A pharmaceutical composition comprising an effective amount of a compound according to claim 1 and a pharmaceutically acceptable carrier.

54. A method of inhibiting MMP-13, comprising administering a compound according to Formula (I): 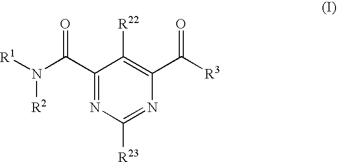
wherein:
R¹ is selected from the group consisting of alkyl, cycloalkyl-alkyl, arylalkyl, heteroarylalkyl and CHR²⁵R²¹ , wherein alkyl, cycloalkyl-alkyl, arylalkyl and heteroarylalkyl are optionally substituted one or more times;
R² is hydrogen;
R³ is NR²⁰R²¹;
R¹⁰ and R¹¹ are independently selected from the group consisting of hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or R¹⁰ and R¹¹ when taken together with the nitrogen to which they are attached complete a 3- to 8-membered ring containing carbon atoms and optionally containing a heteroatom selected from O, S, or NR⁵⁰ and which is optionally substituted one or more times;
R²⁰ is selected from the group consisting of hydrogen and alkyl, wherein alkyl is optionally substituted one or more times;
R²¹ is a bicyclic or tricyclic fused ring system, wherein at least one ring is partially saturated, and wherein said bicyclic or tricyclic fused ring system is optionally substituted one or more times;
R²² and R²³ are independently selected from the group consisting of hydrogen, halo, alkyl, cycloalkyl, hydroxy, alkoxy, aryl, heteroaryl, arylalkyl, heteroarylalkyl, alkenyl, alkynyl, NO₂, NR¹⁰R¹¹, NR¹⁰R¹¹, NR¹⁰N═CR¹⁰R¹¹, NR¹⁰SO₂R¹¹, CN, C(O)OR¹⁰, and fluoroalkyl, wherein alkyl, cycloalkyl, alkoxy, alkenyl, alkynyl and fluoroalkyl are optionally substituted one or more times;
R²⁵ is selected from the group consisting of hydrogen, alkyl, cycloalkyl, C(O)NR¹⁰R¹¹ and haloalkyl, wherein alkyl, cycloalkyl, and haloalkyl are optionally substituted one or more times;
R⁵⁰ is selected from the group consisting of hydrogen, alkyl, aryl, heteroaryl, C(O)R⁸⁰, C(O)NR⁸⁰R⁸¹, SO₂R⁸⁰ and SO₂NR⁸⁰R⁸¹, wherein alkyl, aryl and heteroaryl are optionally substituted one or more times;
R⁸⁰ and R⁸¹ are independently selected from the group consisting of hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or R⁸⁰ and R⁸¹ when taken together with the nitrogen to which they are attached complete a 3- to 8-membered ring containing carbon atoms and optionally a heteroatom selected from O, S(O)ₓ, —NH, and —N(alkyl) and which is optionally substituted one or more times; and
x is selected from 0-2;
or an N-oxide, pharmaceutically acceptable salt or stereoisomer thereof.

55. A method of inhibiting MMP-13, comprising administering a compound according to Formula (IV): 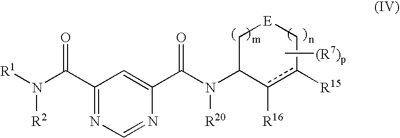
wherein:
R¹ is selected from the group consisting of alkyl, cycloalkyl-alkyl, arylalkyl, heteroarylalkyl and CHR²⁵R²¹, wherein alkyl, cycloalkyl-alkyl, arylalkyl and heteroarylalkyl are optionally substituted one or more times;
R is hydrogen;
R⁴ is selected from the group consisting of R¹⁰, hydrogen, alkyl, aryl, heteroaryl, halo, CF₃, COR¹⁰, OR¹⁰, NR¹⁰R¹¹, NO₂, CN, SO₂OR¹⁰, CO₂R¹⁰, C(O)NR¹⁰, R¹¹, SO₂NR¹⁰R¹¹, SO₂R¹⁰, OC(O)R¹⁰, OC(O)NR¹⁰R¹¹, NR¹⁰C(O)R¹¹, NR¹⁰CO₂R¹¹, (C₀-C₆)-alkyl-C(═NR^{a})NHR^{b}, (C₀-C₆)-alkyl-NHC(═NR^{a})NHR^{b}, (C₀-C₆)-alkyl-C(O)OR¹⁰, (C₀-C₆)-alkyl-C(O)NR¹⁰R¹¹, (C₀-C₆)-alkyl-C(O)—NH—CN, O—(C₀-C₆)-alkyl-C(O)NR¹⁰R¹¹, S(O)ₓ—(C₀-C₆)-alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—SO₂NR¹⁰R¹¹, wherein each R⁴ group is optionally substituted by one or more R¹⁴ groups;
R⁵ is selected from the group consisting of hydrogen, alkyl, C(O)NR¹⁰R¹¹, aryl, arylalkyl, SO₂NR¹⁰R¹¹, C(O)OR¹⁰ and CN, wherein alkyl, aryl and arylalkyl are optionally substituted one or more times;
R⁷ is selected from the group consisting of hydrogen, alkyl, cycloalkyl, halo, R⁴ and NR¹⁰R¹¹, wherein alkyl and cycloalkyl are optionally substituted one or more times;
R¹⁰ and R¹¹ are independently selected from the group consisting of hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or R¹⁰ and R¹¹ when taken together with the nitrogen to which they are attached complete a 3- to 8-membered ring containing carbon atoms and optionally containing a heteroatom selected from O, S, or NR⁵⁰ and which is optionally substituted one or more times;
R¹⁴ is selected from the group consisting of hydrogen, alkyl, arylalkyl, cycloalkyl-alkyl, heteroarylalkyl, heterocyclylalkyl and halo, wherein alkyl, arylalkyl, cycloalkyl-alkyl, heteroarylalkyl and heterocyclylalkyl are optionally substituted one or more times;
R¹⁵ and R¹⁶ when taken together with the carbon atoms to which they are bound, form a ring selected from the group consisting of 6-membered aryl ring, 5- or 6-membered heteroaryl ring, 5- to 8-membered cycloalkyl ring, 5- to 8-membered heterocyclyl ring, 5- to 8-membered cycloalkenyl ring and 5- to 8-membered heterocycloalkenyl ring, wherein said ring is optionally substituted by one or more R⁴groups;
R²⁰ is selected from the group consisting of hydrogen and alkyl, wherein alkyl is optionally substituted one or more times;
R²¹ is a bicyclic or tricyclic fused ring system, wherein at least one ring is partially saturated, and wherein said bicyclic or tricyclic fused ring system is optionally substituted one or more times;
R²⁵ is selected from the group consisting of hydrogen, alkyl, cycloalkyl, C(O)NR¹⁰R¹¹ and haloalkyl, wherein alkyl, cycloalkyl, and haloalkyl are optionally substituted one or more times;
R⁵⁰ is selected from the group consisting of hydrogen, alkyl, aryl, heteroaryl, C(O)R⁸⁰, C(O)NR⁸⁰R⁸¹, SO₂R⁸⁰ and SO₂NR⁸⁰R⁸¹, wherein alkyl, aryl and heteroaryl are optionally substituted one or more times;
R⁸⁰ and R⁸¹ are independently selected from the group consisting of hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or R⁸⁰ and R⁸¹ when taken together with the nitrogen to which they are attached complete a 3- to 8-membered ring containing carbon atoms and optionally a heteroatom selected from O, S(O)ₓ, —NH, and —N(alkyl) and which is optionally substituted one or more times;
Ra^{a} and R^{b} are independently selected from the group consisting of hydrogen, CN, alkyl, haloalkyl, S(O)ₓNR¹⁰R¹¹, S(O)ₓR¹⁰ and C(O)NR¹⁰R¹¹, wherein alkyl and haloalkyl are optionally substituted one or more times;
E is selected from the group consisting of a bond, CR¹⁰R¹¹, O, NR⁵, S, S═O, S(═O)₂, C(═O), N(R¹⁰)(C═O), (C═O)N(R¹⁰), N(R¹⁰)S(═O)₂, S(═O)₂N(R¹⁰), C═N—OR¹¹, —C(R¹⁰R¹¹)C(R¹⁰R¹¹)—,—CH₂—W— and 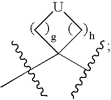
W is selected from the group consisting of O, NRC, S, S═O, S(═O)₂, N(R¹⁰)(C═O), N(R¹⁰)S(═O)₂ and S(═O)₂N(R¹⁰);
U is selected from the group consisting of C(R⁵R¹⁰), NR⁵, O, S, S═O and S(═O)₂;
g and h are independently selected from 0-2;
m and n are independently selected from 0-3, provided that:
(1) when E is present, m and n are not both 3;
(2) when E is —CH₂—W—, m and n are not 3; and
(3) when E is a bond, m and n are not 0;
p is selected from 0-6;
x is selected from 0-2; and
wherein the dotted line represents optionally a double bond;
or an N-oxide, pharmaceutically acceptable salt or stereoisomer thereof.

56. A method of inhibiting MMP-13, comprising administering a compound according to Formula (V): 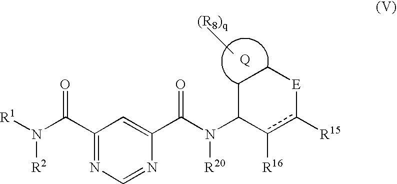
wherein:
R¹ is selected from the group consisting of alkyl, cycloalkyl-alkyl, arylalkyl, heteroarylalkyl and CHR²⁵R²¹, wherein alkyl, cycloalkyl-alkyl, arylalkyl and heteroarylalkyl are optionally substituted one or more times;
R² is hydrogen;
R⁴ is selected from the group consisting of R¹⁰, hydrogen, alkyl, aryl, heteroaryl, halo, CF₃, COR¹⁰, OR¹⁰, NR¹⁰R¹¹, NO₂, CN, SO₂OR¹⁰, CO₂R¹⁰, C(O)NR¹⁰, R¹¹, SO₂NR¹⁰R¹¹, SO₂R¹⁰, OC(O)R¹⁰, OC(O)NR¹⁰R¹¹, NR¹⁰C(O)R¹¹, NR¹⁰CO₂R¹¹, (C₀-C₆)-alkyl-C(═NR^{a})NHR^{b}, C₀-C₆)-alkyl-NHC(═NR^{a})NHR^{b}, (C₀-C₆)-alkyl-C(O)OR¹⁰, (C₀-C₆)-alkyl-C(O)NR¹⁰R¹¹, (C₀-C₆)-alkyl-C(O)—NH—CN, O—(C₀-C₆)-alkyl-C(O)NR¹⁰R¹¹, S(O)ₓ—(C₀-C₆)-alkyl-C(O)OR¹⁰, S(O)_{—(C}₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—SO₂NR¹⁰R , wherein each R⁴ group is optionally substituted by one or more R¹⁴ groups;
R⁵ is selected from the group consisting of hydrogen, alkyl, C(O)NR¹⁰R¹¹, aryl, arylalkyl, SO₂NR¹⁰R¹¹, C(O)OR¹⁰ and CN, wherein alkyl, aryl and arylalkyl are optionally substituted one or more times;
R⁸ is selected from the group consisting of hydrogen, alkyl, OR¹⁰, NR¹⁰R¹¹, CN, arylalkyl, cycloalkyl-alkyl, heteroarylalkyl, heterocyclylalkyl and halo, wherein alkyl, arylalkyl, cycloalkyl-alkyl, heteroarylalkyl and heterocyclylalkyl are optionally substituted one or more times;
R¹⁰ and R¹¹ are independently selected from the group consisting of hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or R¹⁰ and R¹¹ when taken together with the nitrogen to which they are attached complete a 3- to 8-membered ring containing carbon atoms and optionally containing a heteroatom selected from O, S, or NR⁵⁰ and which is optionally substituted one or more times;
R¹⁴ is selected from the group consisting of hydrogen, alkyl, arylalkyl, cycloalkyl-alkyl, heteroarylalkyl, heterocyclylalkyl and halo, wherein alkyl, arylalkyl, cycloalkyl-alkyl, heteroarylalkyl and heterocyclylalkyl are optionally substituted one or more times;
R¹⁵ and R¹⁶ when taken together with the carbon atoms to which they are bound, form a ring selected from the group consisting of 6-membered aryl ring, 5- or 6-membered heteroaryl ring, 5- to 8-membered cycloalkyl ring, 5- to 8-membered heterocyclyl ring, 5- to 8-membered cycloalkenyl ring and 5- to 8-membered heterocycloalkenyl ring, wherein said ring is optionally substituted by one or more R⁴groups;
R²⁰ is selected from the group consisting of hydrogen and alkyl, wherein alkyl is optionally substituted one or more times;
R²¹ is a bicyclic or tricyclic fused ring system, wherein at least one ring is partially saturated, and wherein said bicyclic or tricyclic fused ring system is optionally substituted one or more times;
R²⁵ is selected from the group consisting of hydrogen, alkyl, cycloalkyl, C(O)NR¹⁰R¹¹ and haloalkyl, wherein alkyl, cycloalkyl, and haloalkyl are optionally substituted one or more times;
R⁵⁰ is selected from the group consisting of hydrogen, alkyl, aryl, heteroaryl, C(O)R⁸⁰, C(O)NR⁸⁰R⁸¹, SO₂R⁸⁰ and SO₂NR⁸⁰R⁸¹, wherein alkyl, aryl and heteroaryl are optionally substituted one or more times;
R⁸⁰ and R⁸¹ are independently selected from the group consisting of hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or R⁸⁰ and R⁸¹ when taken together with the nitrogen to which they are attached complete a 3- to 8-membered ring containing carbon atoms and optionally a heteroatom selected from O, S(O)ₓ, —NH, and —N(alkyl) and which is optionally substituted one or more times;
Ra^{a} and R^{b} are independently selected from the group consisting of hydrogen, CN, alkyl, haloalkyl, S(O)ₓNR¹⁰R¹¹, S(O)ₓR¹⁰ and C(O)NR¹⁰R¹¹, wherein alkyl and haloalkyl are optionally substituted one or more times;
E is selected from the group consisting of a bond, CR¹⁰R¹¹, O, NR⁵, S, S═O, S(═O)₂, C(═O), N(R¹⁰)(C═O), (C═O)N(R¹⁰), N(R¹⁰)S(═O)₂, S(═O)₂N(R¹⁰), C═N—OR¹¹, —C(R¹⁰R¹¹)C(R¹⁰R¹¹)—,—CH₂—W— and 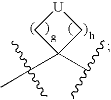
W is selected from the group consisting of O, NR⁵, S, S═O, S(═O)₂, N(R¹⁰)(C═O), N(R¹⁰)S(═O)₂ and S(═O)₂N(R¹⁰);
U is selected from the group consisting of C(R⁵R¹⁰), NR⁵, O, S, S═O and S(═O)₂;
Q is selected from the group consisting of 3-7 membered cycloalkyl, 4-7 membered heterocyclyl, 5-6 membered heteroaryl and 6 membered aryl;
g and h are independently selected from 0-2;
q is selected from 0-4;
x is selected from 0-2; and
wherein the dotted line represents optionally a double bond;
or an N-oxide, pharmaceutically acceptable salt or stereoisomer thereof

57. A method of treating an MMP-13 mediated disease, comprising administering to a patient in need of treatment an effective amount of a compound according to Formula (I): 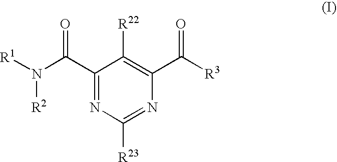
wherein:
R¹ is selected from the group consisting of alkyl, cycloalkyl-alkyl, arylalkyl, heteroarylalkyl and CHR²⁵R²¹, wherein alkyl, cycloalkyl-alkyl, arylalkyl and heteroarylalkyl are optionally substituted one or more times;
R² is hydrogen;
R³ is NR²⁰R²¹;
R¹⁰ and R¹¹ are independently selected from the group consisting of hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or R¹⁰ and R¹¹ when taken together with the nitrogen to which they are attached complete a 3- to 8-membered ring containing carbon atoms and optionally containing a heteroatom selected from O, S, or NR⁵⁰ and which is optionally substituted one or more times;
R²⁰ is selected from the group consisting of hydrogen and alkyl, wherein alkyl is optionally substituted one or more times;
R²¹ is a bicyclic or tricyclic fused ring system, wherein at least one ring is partially saturated, and wherein said bicyclic or tricyclic fused ring system is optionally substituted one or more times;
R²² and R²³ are independently selected from the group consisting of hydrogen, halo, alkyl, cycloalkyl, hydroxy, alkoxy, aryl, heteroaryl, arylalkyl, heteroarylalkyl, alkenyl, alkynyl, NO₂, NR¹⁰R¹¹, NR¹⁰NR¹¹, NR¹⁰═CR¹⁰R¹¹, NR¹⁰SO₂R¹¹, CN, C(O)OR¹⁰, and fluoroalkyl, wherein alkyl, cycloalkyl, alkoxy, alkenyl, alkynyl and fluoroalkyl are optionally substituted one or more times;
R²⁵ is selected from the group consisting of hydrogen, alkyl, cycloalkyl, C(O)NR¹⁰R¹¹ and haloalkyl, wherein alkyl, cycloalkyl, and haloalkyl are optionally substituted one or more times;
R⁵⁰ is selected from the group consisting of hydrogen, alkyl, aryl, heteroaryl, C(O)R⁸⁰, C(O)NR⁸⁰R⁸¹, SO₂R⁸⁰ and SO₂NR⁸⁰R⁸¹, wherein alkyl, aryl and heteroaryl are optionally substituted one or more times;
R⁸⁰ and R⁸¹ are independently selected from the group consisting of hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or R⁸⁰ and R⁸¹ when taken together with the nitrogen to which they are attached complete a 3- to 8-membered ring containing carbon atoms and optionally a heteroatom selected from O, S(O)ₓ, —NH, and —N(alkyl) and which is optionally substituted one or more times;
x is selected from 0-2;
or an N-oxide, pharmaceutically acceptable salt or stereoisomer thereof.

58. A method of treating an MMP-13 mediated disease, comprising administering to a patient in need of treatment an effective amount of a compound according to Formula (IV): 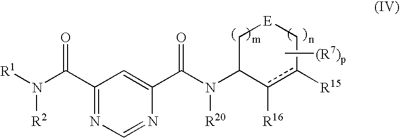
wherein:
R¹ is selected from the group consisting of alkyl, cycloalkyl-alkyl, arylalkyl, heteroarylalkyl and CHR²⁵R²¹, wherein alkyl, cycloalkyl-alkyl, arylalkyl and heteroarylalkyl are optionally substituted one or more times;
R² is hydrogen;
R⁴ is selected from the group consisting of R¹⁰, hydrogen, alkyl, aryl, heteroaryl, halo, CF₃, COR¹⁰, OR¹⁰, NR¹⁰R¹¹, NO₂, CN, SO₂OR¹⁰, CO₂R¹⁰, C(O)NR¹⁰R¹¹, SO₂NR¹⁰R¹¹, SO₂R¹⁰, OC(O)R¹⁰, OC(O)NR¹⁰R¹¹, NR¹⁰C(O)R¹¹, NR¹⁰CO₂R¹¹, (C₀-C₆)-alkyl-C(═NR^{a})NHR^{b}, (C₀-C₆)-alkyl-NHC(═NR^{a})NHR^{b}, (C₀-C₆)-alkyl-C(O)OR¹⁰, (C₀-C₆)-alkyl-C(O)NR¹⁰R¹¹, (C₀-C₆)-alkyl-C(O)—NH—CN, O—(C₀-C₆)-alkyl-C(O)NR¹⁰R¹¹, S(O)ₓ—(C₀-C₆)-alkyl-C(O)OR¹⁰, S(O)ₓ—(C₀-C₆)-alkyl-C(O)NR¹⁰OR¹¹, (C₀-C₆)-alkyl-C(O)NR¹⁰—(C₀-C₆)-alkyl-NR¹⁰R¹¹, (C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—SO₂NR¹⁰R¹¹, wherein each R⁴ group is optionally substituted by one or more R¹⁴ groups;
R⁵ is selected from the group consisting of hydrogen, alkyl, C(O)NR¹⁰R¹¹, aryl, arylalkyl, SO₂NR¹⁰R¹¹, C(O)OR¹⁰ and CN, wherein alkyl, aryl and arylalkyl are optionally substituted one or more times;
R⁷ is selected from the group consisting of hydrogen, alkyl, cycloalkyl, halo, R⁴ and NR¹⁰R¹¹, wherein alkyl and cycloalkyl are optionally substituted one or more times;
R¹⁰ and R¹¹ are independently selected from the group consisting of hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or R¹⁰ and R¹¹ when taken together with the nitrogen to which they are attached complete a 3- to 8-membered ring containing carbon atoms and optionally containing a heteroatom selected from O, S, or NR⁵⁰ and which is optionally substituted one or more times;
R¹⁴ is selected from the group consisting of hydrogen, alkyl, arylalkyl, cycloalkyl-alkyl, heteroarylalkyl, heterocyclylalkyl and halo, wherein alkyl, arylalkyl, cycloalkyl-alkyl, heteroarylalkyl and heterocyclylalkyl are optionally substituted one or more times;
R¹⁵ and R¹⁶ when taken together with the carbon atoms to which they are bound, form a ring selected from the group consisting of 6-membered aryl ring, 5- or 6-membered heteroaryl ring, 5- to 8-membered cycloalkyl ring, 5- to 8-membered heterocyclyl ring, 5- to 8-membered cycloalkenyl ring and 5- to 8-membered heterocycloalkenyl ring, wherein said ring is optionally substituted by one or more R⁴ groups;
R²⁰ is selected from the group consisting of hydrogen and alkyl, wherein alkyl is optionally substituted one or more times;
R²¹ is a bicyclic or tricyclic fused ring system, wherein at least one ring is partially saturated, and wherein said bicyclic or tricyclic fused ring system is optionally substituted one or more times;
R²⁵ is selected from the group consisting of hydrogen, alkyl, cycloalkyl, C(O)NR¹⁰R¹¹ and haloalkyl, wherein alkyl, cycloalkyl, and haloalkyl are optionally substituted one or more times;
R⁵⁰ is selected from the group consisting of hydrogen, alkyl, aryl, heteroaryl, C(O)R⁸⁰, C(O)NR⁸⁰R⁸¹, SO₂R⁸⁰ and SO₂NR⁸⁰R⁸¹, wherein alkyl, aryl and heteroaryl are optionally substituted one or more times;
R⁸⁰ and R⁸¹ are independently selected from the group consisting of hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or R⁸⁰ and R⁸¹ when taken together with the nitrogen to which they are attached complete a 3- to 8-membered ring containing carbon atoms and optionally a heteroatom selected from O, S(O)ₓ, —NH, and —N(alkyl) and which is optionally substituted one or more times;
Ra^{a} and R^{b} are independently selected from the group consisting of hydrogen, CN, alkyl, haloalkyl, S(O)ₓNR¹⁰R¹¹, S(O)ₓR¹⁰ and C(O)NR¹⁰R¹¹, wherein alkyl and haloalkyl are optionally substituted one or more times;
E is selected from the group consisting of a bond, CR¹⁰R¹¹, O, NR⁵, S, S═O, S(═O)₂, C(═O), N(R¹⁰)(C═O), (C═O)N(R¹⁰), N(R¹⁰)S(═O)₂, S(═O)₂N(R¹⁰), C═N—OR¹¹, C(R¹⁰R¹¹)C(R¹⁰R¹¹)—,—CH₂—W— and —C(R¹⁰R¹¹)C(R¹⁰R¹¹)—, —CH₂—W— and 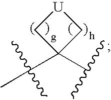
W is selected from the group consisting of O, NR⁵, S, S═O, S(═O)₂, N(R¹⁰)(C═O), N(R¹⁰)S(═O)₂ and S(═O)₂N(R¹⁰);
U is selected from the group consisting of C(R⁵R¹⁰), NR⁵, O, S, S═O and S(═O)₂;
g and h are independently selected from 0-2;
m and n are independently selected from 0-3, provided that:
(1) when E is present, m and n are not both 3;
(2) when E is —CH₂-W-, m and n are not 3; and
(3) when E is a bond, m and n are not 0;
p is selected from 0-6;
x is selected from 0-2; and
wherein the dotted line represents optionally a double bond;
or an N-oxide, pharmaceutically acceptable salt or stereoisomer thereof.

59. A method of treating an MMP-13 mediated disease, comprising administering to a patient in need of treatment an effective amount of a compound according to Formula (V): 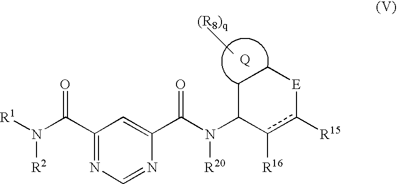
wherein:
R¹ is selected from the group consisting of alkyl, cycloalkyl-alkyl, arylalkyl, heteroarylalkyl and CHR²⁵R²¹, wherein alkyl cycloalkyl-alkyl, arylalkyl and heteroarylalkyl are optionally substituted one or more times;
R² is hydrogen;
R⁴ is selected from the group consisting of R¹⁰, hydrogen, alkyl, aryl, heteroaryl, halo, CF₃, COR¹⁰, OR¹⁰, NR¹⁰R¹¹, NO₂, CN, SO₂OR¹⁰, CO₂R¹⁰, C(O)NR¹⁰, R¹¹, SO₂NR¹⁰R¹¹, SO₂R¹⁰, OC(O)R¹⁰, OC(O)NR¹⁰R¹¹, NR¹⁰C(O)R¹¹, NR¹⁰CO₂R¹¹, (C₀-C₆)-alkyl-C(═NR^{a})NHR^{b},(C₀-C₆)-alkyl-NHC(═NR^{a})NHR^{b}, (C₀-C₆)-alkyl-C(O)OR¹⁰, (C₀-C₆)-alkyl-C(O)NR¹⁰R¹¹, (C₀-C₆)-alkyl-C(O)—NH—CN, O—(C₀-C₆)-alkyl-C(O)NR¹⁰R¹¹, S(O)ₓ—(C₀-C₆)-alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—SO₂NR¹⁰R¹¹, wherein each R⁴ group is optionally substituted by one or more R¹⁴ groups;
R⁵ is selected from the group consisting of hydrogen, alkyl, C(O)NR¹⁰R¹¹, aryl, arylalkyl, SO₂NR¹⁰R¹¹, C(O)OR¹⁰ and CN, wherein alkyl, aryl and arylalkyl are optionally substituted one or more times;
R⁸ is selected from the group consisting of hydrogen, alkyl, OR¹⁰, NR¹⁰R¹¹, CN, arylalkyl, cycloalkyl-alkyl, heteroarylalkyl, heterocyclylalkyl and halo, wherein alkyl, arylalkyl, cycloalkyl-alkyl, heteroarylalkyl and heterocyclylalkyl are optionally substituted one or more times;
R¹⁰ and R¹¹ are independently selected from the group consisting of hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or R¹⁰ and R¹¹ when taken together with the nitrogen to which they are attached complete a 3- to 8-membered ring containing carbon atoms and optionally containing a heteroatom selected from O, S, or NR⁵⁰ and which is optionally substituted one or more times;
R¹⁴ is selected from the group consisting of hydrogen, alkyl, arylalkyl, cycloalkyl-alkyl, heteroarylalkyl, heterocyclylalkyl and halo, wherein alkyl, arylalkyl, cycloalkyl-alkyl, heteroarylalkyl and heterocyclylalkyl are optionally substituted one or more times;
R¹⁵ and R¹⁶ when taken together with the carbon atoms to which they are bound, form a ring selected from the group consisting of 6-membered aryl ring, 5- or 6-membered heteroaryl ring, 5- to 8-membered cycloalkyl ring, 5- to 8-membered heterocyclyl ring, 5- to 8-membered cycloalkenyl ring and 5- to 8-membered heterocycloalkenyl ring, wherein said ring is optionally substituted by one or more R⁴groups;
R²⁰ is selected from the group consisting of hydrogen and alkyl, wherein alkyl is optionally substituted one or more times;
R²¹ is a bicyclic or tricyclic fused ring system, wherein at least one ring is partially saturated, and wherein said bicyclic or tricyclic fused ring system is optionally substituted one or more times;
R²⁵ is selected from the group consisting of hydrogen, alkyl, cycloalkyl, C(O)NR¹⁰R¹¹ and haloalkyl, wherein alkyl, cycloalkyl, and haloalkyl are optionally substituted one or more times;
R⁵⁰ is selected from the group consisting of hydrogen, alkyl, aryl, heteroaryl, C(O)R⁸⁰, C(O)NR⁸⁰R⁸¹, SO₂R⁸⁰ and SO₂NR⁸⁰R⁸¹, wherein alkyl, aryl and heteroaryl are optionally substituted one or more times;
R⁸⁰ and R⁸¹ are independently selected from the group consisting of hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or R⁸⁰ and R⁸¹ when taken together with the nitrogen to which they are attached complete a 3- to 8-membered ring containing carbon atoms and optionally a heteroatom selected from O, S(O)ₓ, —NH, and —N(alkyl) and which is optionally substituted one or more times;
Ra^{a} and R^{b} are independently selected from the group consisting of hydrogen, CN, alkyl, haloalkyl, S(O)ₓNR¹⁰R¹¹, S(O)ₓR¹⁰ and C(O)NR¹⁰R¹¹, wherein alkyl and haloalkyl are optionally substituted one or more times;
E is selected from the group consisting of a bond, CR¹⁰R¹¹, O, NR⁵, S, S═O, S(═O )₂, C(═O), N(R¹⁰)(C═O), (C═O)N(R¹⁰), N(R¹⁰)S(═O )₂, S(═O )₂N(R¹⁰), C═N—OR¹¹, —C(R¹⁰R¹¹)C(R¹⁰R¹¹)—,—CH₂—W— and 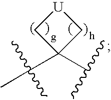
W is selected from the group consisting of O, NR⁵, S, S═O, S(═O)₂, N(R¹⁰)(C═O), N(R¹⁰)S(═O)₂ and S(═O)₂N(R¹⁰);
U is selected from the group consisting of C(R⁵R¹⁰), NR⁵, O, S, S═O and S(═O)₂;
Q is selected from the group consisting of 3-7 membered cycloalkyl, 4-7 membered heterocyclyl, 5-6 membered heteroaryl and 6 membered aryl;
g and h are independently selected from 0-2;
q is selected from 0-4;
x is selected from 0-2; and
wherein the dotted line represents optionally a double bond;
or an N-oxide, pharmaceutically acceptable salt or stereoisomer thereof.

60. The method according to claim 57, wherein the disease is rheumatoid arthritis.

61. The method according to claim 57, wherein the disease is osteoarthritis.

62. The method according to claim 57, wherein the disease is abdominal aortic aneurysm.

63. The method according to claim 57, wherein the disease is cancer.

64. The method according to claim 57, wherein the disease is inflammation.

65. The method according to claim 57, wherein the disease is atherosclerosis.

66. The method according to claim 57, wherein the disease is multiple sclerosis.

67. The method according to claim 57, wherein the disease is chronic obstructive pulmonary disease.

68. The method according to claim 57, wherein the disease is pain.

69. The method according to claim 57, wherein the disease is inflammatory pain.

70. The method according to claim 57, wherein the disease is bone pain.

71. The method according to claim 57, wherein the disease is joint pain.

72. The method according to claim 58, wherein the disease is rheumatoid arthritis.

73. The method according to claim 58, wherein the disease is osteoarthritis.

74. The method according to claim 58, wherein the disease is abdominal aortic aneurysm.

75. The method according to claim 58, wherein the disease is cancer.

76. The method according to claim 58, wherein the disease is inflammation.

77. The method according to claim 58, wherein the disease is atherosclerosis.

78. The method according to claim 58, wherein the disease is multiple sclerosis.

79. The method according to claim 58, wherein the disease is chronic obstructive pulmonary disease.

80. The method according to claim 58, wherein the disease is pain.

81. The method according to claim 58, wherein the disease is inflammatory pain.

82. The method according to claim 58, wherein the disease is bone pain.

83. The method according to claim 58, wherein the disease is joint pain.

84. The method according to claim 59, wherein the disease is rheumatoid arthritis.

85. The method according to claim 59, wherein the disease is osteoarthritis.

86. The method according to claim 59, wherein the disease is abdominal aortic aneurysm.

87. The method according to claim 59, wherein the disease is cancer.

88. The method according to claim 59, wherein the disease is inflammation.

89. The method according to claim 59, wherein the disease is atherosclerosis.

90. The method according to claim 59, wherein the disease is multiple sclerosis.

91. The method according to claim 59, wherein the disease is chronic obstructive pulmonary disease.

92. The method according to claim 59, wherein the disease is pain.

93. The method according to claim 59, wherein the disease is inflammatory pain.

94. The method according to claim 59, wherein the disease is bone pain.

95. The method according to claim 59, wherein the disease is joint pain.

96. A pharmaceutical composition comprising an effective amount of a compound according to claim 1, a pharmaceutically acceptable carrier and a drug, agent or therapeutic selected from the group consisting of: (a) a disease modifying antirheumatic drug; (b) a nonsteroidal anti-inflammatory drug; (c) a COX-2 selective inhibitor; (d) a COX-1 inhibitor; (e) an immunosuppressive; (f) a steroid; (g) a biological response modifier; and (h) other anti-inflammatory agents or therapeutics useful for the treatment of chemokine mediated diseases.

97. The pharmaceutical composition according to claim 96, wherein said COX-2 selective inhibitor is selected from the group consisting of rofecoxib, celecoxib, and valdecoxib.

98. The pharmaceutical composition according to claim 96, wherein said COX-1 inhibitor is piroxicam.

99. A pharmaceutical composition comprising an effective amount of a compound according to claim 24, a pharmaceutically acceptable carrier and a drug, agent or therapeutic selected from the group consisting of: (a) a disease modifying antirheumatic drug; (b) a nonsteroidal anti-inflammatory drug; (c) a COX-2 selective inhibitor; (d) a COX-1 inhibitor; (e) an immunosuppressive; (f) a steroid; (g) a biological response modifier; and (h) other anti-inflammatory agents or therapeutics useful for the treatment of chemokine mediated diseases.

100. The pharmaceutical composition according to claim 99, wherein said COX-2 selective inhibitor is selected from the group consisting of rofecoxib, celecoxib, and valdecoxib.

101. The pharmaceutical composition according to claim 99, wherein said COX-1 inhibitor is piroxicam.

102. A pharmaceutical composition comprising an effective amount of a compound according to claim 25, a pharmaceutically acceptable carrier and a drug, agent or therapeutic selected from the group consisting of: (a) a disease modifying antirheumatic drug; (b) a nonsteroidal anti-inflammatory drug; (c) a COX-2 selective inhibitor; (d) a COX-1 inhibitor; (e) an immunosuppressive; (f) a steroid; (g) a biological response modifier; and (h) other anti-inflammatory agents or therapeutics useful for the treatment of chemokine mediated diseases.

103. The pharmaceutical composition according to claim 96, wherein said COX-2 selective inhibitor is selected from the group consisting of rofecoxib, celecoxib, and valdecoxib.

104. The pharmaceutical composition according to claim 96, wherein said COX-1 inhibitor is piroxicam.

105. The method according to claim 57, wherein the disease is selected from the group consisting of: rheumatoid arthritis, osteoarthritis, abdominal aortic aneurysm, cancer, inflammation, atherosclerosis, multiple sclerosis, chronic obstructive pulmonary disease, ocular diseases, neurologic diseases, psychiatric diseases, thrombosis, bacterial infection, Parkinson's disease, fatigue, tremor, diabetic retinopathy, vascular diseases of the retina, aging, dementia, cardiomyopathy, renal tubular impairment, diabetes, psychosis, dyskinesia, pigmentary abnormalities, deafness, inflammatory and fibrotic syndromes, intestinal bowel syndrome, allergies, Alzheimers disease, arterial plaque formation, viral infection, stroke, atherosclerosis, cardiovascular disease, reperfusion injury, trauma, chemical exposure or oxidative damage to tissues, pain, inflammatory pain, bone pain and joint pain.

106. The method according to claim 58, wherein the disease is selected from the group consisting of: rheumatoid arthritis, osteoarthritis, abdominal aortic aneurysm, cancer, inflammation, atherosclerosis, multiple sclerosis, chronic obstructive pulmonary disease, ocular diseases, neurologic diseases, psychiatric diseases, thrombosis, bacterial infection, Parkinson's disease, fatigue, tremor, diabetic retinopathy, vascular diseases of the retina, aging, dementia, cardiomyopathy, renal tubular impairment, diabetes, psychosis, dyskinesia, pigmentary abnormalities, deafness, inflammatory and fibrotic syndromes, intestinal bowel syndrome, allergies, Alzheimers disease, arterial plaque formation, viral infection, stroke, atherosclerosis, cardiovascular disease, reperfusion injury, trauma, chemical exposure or oxidative damage to tissues, pain, inflammatory pain, bone pain and joint pain.

107. The method according to claim 59, wherein the disease is selected from the group consisting of: rheumatoid arthritis, osteoarthritis, abdominal aortic aneurysm, cancer, inflammation, atherosclerosis, multiple sclerosis, chronic obstructive pulmonary disease, ocular diseases, neurologic diseases, psychiatric diseases, thrombosis, bacterial infection, Parkinson's disease, fatigue, tremor, diabetic retinopathy, vascular diseases of the retina, aging, dementia, cardiomyopathy, renal tubular impairment, diabetes, psychosis, dyskinesia, pigmentary abnormalities, deafness, inflammatory and fibrotic syndromes, intestinal bowel syndrome, allergies, Alzheimers disease, arterial plaque formation, viral infection, stroke, atherosclerosis, cardiovascular disease, reperfusion injury, trauma, chemical exposure or oxidative damage to tissues, pain, inflammatory pain, bone pain and joint pain.
